(19) United States
(12) Patent Application Publication
(10) Pub. No.: US 2026/0144898 A1
(43) Pub. Date: May 28, 2026

(54) GENE EDITING SYSTEMS AND USES THEREOF

(71) Applicant: CORRECTSEQUENCE THERAPEUTICS (SHANGHAI) CO., LTD, Shanghai (CN)

(72) Inventors: Lijie WANG, Shanghai (CN); Yichuan WANG, Shanghai (CN); Peixue LI, Shanghai (CN); Lang ZHANG, Shanghai (CN); Xiaodun MOU, Shanghai (CN)

(21) Appl. No.: 19/376,282

(22) Filed: Oct. 31, 2025

Related U.S. Application Data
(63) Continuation of application No. PCT/CN2024/095200, filed on May 24, 2024.

(30) Foreign Application Priority Data
May 26, 2023 (WO) PCT/CN2023/096482

(51) Int. Cl. A61K 48/00; C12N 9/22; C12N 9/78; C12N 15/11; C12N 15/90

(52) U.S. Cl. CPC A61K 48/0058; A61K 48/0016; C12N 9/226; C12N 9/78; C12N 15/11; C12N 15/907; C12N 2310/20; C12N 2310/315; C12N 2310/321

(57) ABSTRACT

The present disclosure generally relates to a LigoRNA system, and LigoRNA-based gene editing systems and uses thereof. Specifically, this disclosure provides novel base editing systems to achieve genomic editing at the base level, which enables broad applications in life science research and clinical therapy and/or biotechnology development. Also disclosed are polynucleotides, vectors, cells, and kits comprising components of the gene editing systems.

### CROSS REFERENCE TO RELATED APPLICATIONS

This application is a continuation of International Application No. PCT/CN2024/095200, filed on May 24, 2024, which claims the priority to and benefits of International Application No. PCT/CN2023/096482, filed on May 26, 2023. Both aforementioned applications are incorporated herein by reference in their entireties.

### FIELD OF DISCLOSURE

The present disclosure generally relates to a LigoRNA system and LigoRNA-based gene editing systems and uses thereof. Specifically, this disclosure provides novel base editing systems to achieve genomic editing at the base level, which enables broad applications in life science research and clinical therapy and/or biotechnology development. Also disclosed are polynucleotides, vectors, cells, and kits comprising components of the gene editing systems.

### SEQUENCE LISTING

This application contains a Sequence Listing electronically submitted as an XML file entitled “SL16881-0002RE.xml” having a size of 569,142 bytes and created on Sep. 18, 2025. The information contained in the Sequence Listing is incorporated by reference herein in its entirety.

### BACKGROUND

(CRISPR)/CRISPR-associated (Cas) systems, derived from evolved adaptive immune defense system of bacteria and archaea, have been widely applied to gene editing and regulation. Cas9 nucleases can be directed by single guide RNAs (sgRNAs) to target and induce cleavage at endogenous genomic loci in various species. The combination of CRISPR-Cas9 and cytidine deaminases leads to cytosine base editors (CBEs) for programmable cytosine to thymine (C-to-T) substitution. The combination of CRISPR-Cas9 and adenosine deaminases leads to adenine base editors (ABEs) for programmable adenine to guanine (A-to-G) substitution. Both Cas nucleases and base editors have been applied to gene editing and hold great potentials in clinical applications. Currently, CRISPR ribonucleoprotein (RNP) or mRNA with sgRNA have been regularly used for genome editing through nucleofection or lipid nanoparticles delivery system. Using these RNA systems for gene editing has many advantages over the traditional plasmid method, including improved transfection efficiency in hard-to-transfect cells and reduced off-target effects.

A transformer base editor (tBE) system has been described by Wang, L. et al., 2021, the content of which is incorporated herein by reference. The tBE system comprises a cytidine deaminase inhibitor (dCDI) domain and a split-TEV protease. Only when binding at on-target sites, tBE is transformed to cleave off the dCDI domain and catalyzes targeted deamination for editing.

In the tBE system previously described by Wang, L. et al., two single guide RNAs were used to achieve base editing. For example, the tBE system comprises a helper sgRNA (hsgRNA) containing an MS2 hairpin to recruit APOBEC and dCDI domains, and a main sgRNA (msgRNA) containing boxB hairpins to generate an editing region and recruit a TEV protease. Due to the addition of protein-recruiting hairpins in hsgRNA and msgRNA, the length of these two sgRNAs are over 100 nt. However, chemically synthesized RNA oligonucleotides over 100 nt demonstrate much lower yield and purity, resulting in challenges for large-scale production and cost control. There is a need in the art to solve this problem.

### SUMMARY

In an aspect, the present disclosure provides a new RNA system called ligand-bound RNA (LigoRNA) which does not require long sgRNAs, and thus avoids the difficulty of synthesizing long RNA oligonucleotides. The LigoRNA system can reduce the cost of relevant research and therapeutics. The LigoRNA system comprises a dual-RNA structure formed by a ligand-bound CRISPR RNA (crRNA) and a trans-activating crRNA (tracrRNA). For example, the LigoRNA system comprises a helper guide-RNA (hgRNA) set of a helper crRNA (hcrRNA) and a tracrRNA, and a main gRNA (mgRNA) set of a main crRNA (mcrRNA) and a tracrRNA.

The tracrRNA and the ligand-bound crRNA can form a dual-RNA structure, which can target a nucleotide sequence. Site-specific editing occurs at locations determined by both base-pairing complementarity between the crRNA and the target DNA, and the binding of Cas protein at the protospacer adjacent motif (PAM).

In an aspect, the present disclosure provides an engineered CRISPR RNA (crRNA) comprising a spacer sequence and a linker sequence, wherein the linker sequence comprises at least one protein-binding motif, wherein the protein-binding motif is an RNA aptamer motif.

In some embodiments, the protein-binding motif is MS2, PP7, boxB, SfMu hairpin motif, telomerase Ku, or Sm7 binding motif.

In some embodiments of the engineered crRNA described herein, the linker sequence is any one of SEQ ID NOs: 1-3 and 149-151.

In some embodiments of the engineered crRNA described herein, the linker sequence is any one of SEQ ID NOs: 4-7 and 152-153.

In some embodiments of the engineered crRNA described herein, the engineered crRNA is any one of SEQ ID NOs: 13-21, 154-158, 302-307, and 364-395.

In some embodiments of the engineered crRNA described herein, the crRNA is capable of forming a base-pair structure with a trans-activating crRNA (tracrRNA).

In some embodiments of the engineered crRNA described herein, the tracrRNA is any one of SEQ ID NOs: 10-12. In some embodiments, the tracrRNA is any one of SEQ ID NOs: 22-24.

In some embodiments of the engineered crRNA described herein, the engineered crRNA comprises at least one nucleotide with modification. In some embodiments, the modification is selected from 2′-O-alkyl, 2′-substituted alkoxy, 2′-substituted alkyl, 2′-halo, 3′-phosphorothioate, bridged nucleic acid (BNA), and locked nucleic acid (LNA). In some embodiments, the at least one nucleotide with modification is any one of the first three nucleotides from 3′-end of the engineered crRNA.

In an aspect, the present disclosure provides an engineered trans-activating crRNA (tracrRNA) of SEQ ID NO: 11 or SEQ ID NO: 12.

In an aspect, the present disclosure provides an engineered trans-activating crRNA (tracrRNA) of any one of SEQ ID NOs: 22-24.

In some embodiments of the engineered tracrRNA described herein, the engineered tracrRNA comprises at least one nucleotide with modification. In some embodiments, the modification is selected from 2′-O-alkyl, 2′-substituted alkoxy, 2′-substituted alkyl, 2′-halo, 3′-phosphorothioate, bridged nucleic acid (BNA), and locked nucleic acid (LNA). In some embodiments, the at least one nucleotide with modification is any one of the first three nucleotides from 3′-end of the engineered tracrRNA.

In an aspect, the present disclosure provides a kit comprising an engineered crRNA described herein.

In an aspect, the present disclosure provides a kit comprising a first engineered crRNA described herein of any one of SEQ ID NOs: 1-3 and 149-151, and a second engineered crRNA described herein of any one of SEQ ID NOs: 4-7 and 152-153.

In some embodiments of the kit described herein, comprising a first engineered crRNA and a second engineered crRNA, wherein the first engineered crRNA comprises a first linker sequence and the second engineered crRNA comprises a second linker sequence, and wherein the first linker sequence and the second linker sequence are
a. SEQ ID NO: 1 and SEQ ID NO: 8, respectively; or
b. SEQ ID NO: 1 and SEQ ID NO: 4, respectively; or
c. SEQ ID NO: 2 and SEQ ID NO: 8, respectively; or
d. SEQ ID NO: 2 and SEQ ID NO: 5, respectively; or
e. SEQ ID NO: 3 and SEQ ID NO: 8, respectively; or
f. SEQ ID NO: 3 and SEQ ID NO: 6, respectively; or
g. SEQ ID NO: 1 and SEQ ID NO: 7, respectively; or
h. SEQ ID NO: 1 and SEQ ID NO: 5, respectively; or
i. SEQ ID NO: 3 and SEQ ID NO: 5, respectively; or
j. SEQ ID NO: 1 and SEQ ID NO: 6, respectively; or
k. SEQ ID NO: 2 and SEQ ID NO: 6, respectively; or
l. SEQ ID NO: 3 and SEQ ID NO: 6, respectively; or
m. SEQ ID NO: 3 and SEQ ID NO: 4, respectively; or
n. SEQ ID NO: 149 and SEQ ID NO: 152, respectively; or
o. SEQ ID NO: 150 and SEQ ID NO: 152, respectively; or
p. SEQ ID NO: 151 and SEQ ID NO: 152, respectively; or
q. SEQ ID NO: 149 and SEQ ID NO: 153, respectively; or
r. SEQ ID NO: 150 and SEQ ID NO: 153, respectively; or
s. SEQ ID NO: 151 and SEQ ID NO: 153, respectively.

In some embodiments of the kit described herein, the kit comprises a first engineered crRNA and a second engineered crRNA, wherein the first engineered crRNA and the second engineered crRNA are
a. SEQ ID NO: 21 and SEQ ID NO: 20, respectively; or
b. SEQ ID NO: 13 and SEQ ID NO: 20, respectively; or
c. SEQ ID NO: 13 and SEQ ID NO: 16, respectively; or
d. SEQ ID NO: 14 and SEQ ID NO: 20, respectively; or
e. SEQ ID NO: 14 and SEQ ID NO: 17, respectively; or
f. SEQ ID NO: 15 and SEQ ID NO: 20, respectively; or
g. SEQ ID NO: 15 and SEQ ID NO: 18, respectively; or
h. SEQ ID NO: 13 and SEQ ID NO: 19, respectively; or
i. SEQ ID NO: 13 and SEQ ID NO: 17, respectively; or
j. SEQ ID NO: 15 and SEQ ID NO: 17, respectively; or
k. SEQ ID NO: 13 and SEQ ID NO: 18, respectively; or
l. SEQ ID NO: 14 and SEQ ID NO: 18, respectively; or
m. SEQ ID NO: 15 and SEQ ID NO: 18, respectively; or
n. SEQ ID NO: 15 and SEQ ID NO: 16, respectively; or
o. SEQ ID NO: 154 and SEQ ID NO: 157, respectively; or
p. SEQ ID NO: 155 and SEQ ID NO: 157, respectively; or
q. SEQ ID NO: 156 and SEQ ID NO: 157, respectively; or
r. SEQ ID NO: 154 and SEQ ID NO: 158, respectively; or
s. SEQ ID NO: 155 and SEQ ID NO: 158, respectively; or
a. SEQ ID NO: 156 and SEQ ID NO: 158, respectively; or
b. SEQ ID NO: 302 and SEQ ID NO: 303, respectively; or
c. SEQ ID NO: 304 and SEQ ID NO: 305, respectively; or
d. SEQ ID NO: 306 and SEQ ID NO: 307, respectively; or
e. SEQ ID NO: 364 and SEQ ID NO: 365, respectively; or
f. SEQ ID NO: 366 and SEQ ID NO: 367, respectively; or
g. SEQ ID NO: 368 and SEQ ID NO: 369, respectively; or
h. SEQ ID NO: 390, and SEQ ID NO: 389, respectively; or
i. SEQ ID NO: 382, and SEQ ID NO: 389, respectively; or
j. SEQ ID NO: 382, and SEQ ID NO: 385, respectively; or
k. SEQ ID NO: 382, and SEQ ID NO: 385, respectively; or
l. SEQ ID NO: 383, and SEQ ID NO: 389, respectively; or
m. SEQ ID NO: 383, and SEQ ID NO: 386, respectively; or
n. SEQ ID NO: 383, and SEQ ID NO: 386, respectively; or
o. SEQ ID NO: 384, and SEQ ID NO: 389, respectively; or
p. SEQ ID NO: 384, and SEQ ID NO: 387, respectively; or
q. SEQ ID NO: 382, and SEQ ID NO: 388, respectively; or
r. SEQ ID NO: 382, and SEQ ID NO: 388, respectively; or
s. SEQ ID NO: 382, and SEQ ID NO: 386, respectively; or
t. SEQ ID NO: 384, and SEQ ID NO: 386, respectively; or
u. SEQ ID NO: 382, and SEQ ID NO: 386, respectively; or
v. SEQ ID NO: 384, and SEQ ID NO: 386, respectively; or
w. SEQ ID NO: 382, and SEQ ID NO: 387, respectively; or
x. SEQ ID NO: 383, and SEQ ID NO: 387, respectively; or
y. SEQ ID NO: 383, and SEQ ID NO: 387, respectively; or
z. SEQ ID NO: 384, and SEQ ID NO: 387, respectively; or
aa. SEQ ID NO: 384, and SEQ ID NO: 385, respectively; or
bb. SEQ ID NO: 370 and SEQ ID NO: 371, respectively; or
cc. SEQ ID NO: 372 and SEQ ID NO: 373, respectively; or
dd. SEQ ID NO: 374 and SEQ ID NO: 375, respectively or
ee. SEQ ID NO: 376 and SEQ ID NO: 377, respectively; or
ff. SEQ ID NO: 378 and SEQ ID NO: 379, respectively; or
gg. SEQ ID NO: 380 and SEQ ID NO: 381, respectively.

In some embodiments of the kit described herein, it further comprises at least one tracrRNA, wherein the at least one tracrRNA are the same or different.

In some embodiments of the kit described herein, each of the at least one tracrRNA is selected from SEQ ID NOs: 10-12. In some embodiments, the tracrRNA is any one of SEQ ID NOs: 22-24.

In some embodiments of the kit described herein, wherein the first engineered crRNA comprises a first linker sequence and the second engineered crRNA comprises a second linker sequence, and wherein the first linker sequence, the second linker sequence, and the tracrRNA are
a. SEQ ID NO: 1, SEQ ID NO: 8, and SEQ ID NO: 10, respectively; or
b. SEQ ID NO: 1, SEQ ID NO: 4, and SEQ ID NO: 10, respectively; or
c. SEQ ID NO: 1, SEQ ID NO: 4, and SEQ ID NO: 11, respectively; or
d. SEQ ID NO: 2, SEQ ID NO: 8, and SEQ ID NO: 10, respectively; or
e. SEQ ID NO: 2, SEQ ID NO: 5, and SEQ ID NO: 10, respectively; or
f. SEQ ID NO: 2, SEQ ID NO: 5, and SEQ ID NO: 12, respectively; or
g. SEQ ID NO: 3, SEQ ID NO: 8, and SEQ ID NO: 10, respectively; or
h. SEQ ID NO: 3, SEQ ID NO: 6, and SEQ ID NO: 10, respectively; or
i. SEQ ID NO: 1, SEQ ID NO: 7, and SEQ ID NO: 10, respectively; or
j. SEQ ID NO: 1, SEQ ID NO: 7, and SEQ ID NO: 11, respectively; or
k. SEQ ID NO: 1, SEQ ID NO: 5, and SEQ ID NO: 10, respectively; or
l. SEQ ID NO: 3, SEQ ID NO: 5, and SEQ ID NO: 10, respectively; or
m. SEQ ID NO: 1, SEQ ID NO: 5, and SEQ ID NO: 12, respectively; or
n. SEQ ID NO: 3, SEQ ID NO: 5, and SEQ ID NO: 12, respectively; or
o. SEQ ID NO: 1, SEQ ID NO: 6, and SEQ ID NO: 10, respectively; or
p. SEQ ID NO: 2, SEQ ID NO: 6, and SEQ ID NO: 10, respectively; or
q. SEQ ID NO: 2, SEQ ID NO: 6, and SEQ ID NO: 11, respectively; or
r. SEQ ID NO: 3, SEQ ID NO: 6, and SEQ ID NO: 11, respectively; or
s. SEQ ID NO: 3, SEQ ID NO: 4, and SEQ ID NO: 11, respectively; or
t. SEQ ID NO: 149, SEQ ID NO: 152, and SEQ ID NO: 10, respectively; or
u. SEQ ID NO: 150, SEQ ID NO: 152, and SEQ ID NO: 10, respectively; or
v. SEQ ID NO: 151, SEQ ID NO: 152, and SEQ ID NO: 10, respectively; or
w. SEQ ID NO: 149, SEQ ID NO: 153, and SEQ ID NO: 10, respectively; or
x. SEQ ID NO: 150, SEQ ID NO: 153, and SEQ ID NO: 10, respectively; or
y. SEQ ID NO: 151, SEQ ID NO: 153, and SEQ ID NO: 10, respectively.

In some embodiments of the kit described herein, the first engineered crRNA, the second engineered crRNA, and the tracrRNA are
a. SEQ ID NO: 21, SEQ ID NO: 20, and SEQ ID NO: 22, respectively; or
b. SEQ ID NO: 13, SEQ ID NO: 20, and SEQ ID NO: 22, respectively; or
c. SEQ ID NO: 13, SEQ ID NO: 16, and SEQ ID NO: 22, respectively; or
d. SEQ ID NO: 13, SEQ ID NO: 16, and SEQ ID NO: 23, respectively; or
e. SEQ ID NO: 14, SEQ ID NO: 20, and SEQ ID NO: 22, respectively; or
f. SEQ ID NO: 14, SEQ ID NO: 17, and SEQ ID NO: 22, respectively; or
g. SEQ ID NO: 14, SEQ ID NO: 17, and SEQ ID NO: 24, respectively; or
h. SEQ ID NO: 15, SEQ ID NO: 20, and SEQ ID NO: 22, respectively; or
i. SEQ ID NO: 15, SEQ ID NO: 18, and SEQ ID NO: 22, respectively; or
j. SEQ ID NO: 13, SEQ ID NO: 19, and SEQ ID NO: 22, respectively; or
k. SEQ ID NO: 13, SEQ ID NO: 19, and SEQ ID NO: 23, respectively; or
l. SEQ ID NO: 13, SEQ ID NO: 17, and SEQ ID NO: 22, respectively; or
m. SEQ ID NO: 15, SEQ ID NO: 17, and SEQ ID NO: 22, respectively; or
n. SEQ ID NO: 13, SEQ ID NO: 17, and SEQ ID NO: 24, respectively; or
o. SEQ ID NO: 15, SEQ ID NO: 17, and SEQ ID NO: 24, respectively; or
p. SEQ ID NO: 13, SEQ ID NO: 18, and SEQ ID NO: 22, respectively; or
q. SEQ ID NO: 14, SEQ ID NO: 18, and SEQ ID NO: 22, respectively; or
r. SEQ ID NO: 14, SEQ ID NO: 18, and SEQ ID NO: 23, respectively; or
s. SEQ ID NO: 15, SEQ ID NO: 18, and SEQ ID NO: 23, respectively; or
t. SEQ ID NO: 15, SEQ ID NO: 16, and SEQ ID NO: 23, respectively; or
u. SEQ ID NO: 154, SEQ ID NO: 157, and SEQ ID NO: 22, respectively; or
v. SEQ ID NO: 155, SEQ ID NO: 157, and SEQ ID NO: 22, respectively; or
w. SEQ ID NO: 156, SEQ ID NO: 157, and SEQ ID NO: 22, respectively; or
x. SEQ ID NO: 154, SEQ ID NO: 158, and SEQ ID NO: 22, respectively; or
y. SEQ ID NO: 155, SEQ ID NO: 158, and SEQ ID NO: 22, respectively; or
hh. SEQ ID NO: 156, SEQ ID NO: 158, and SEQ ID NO: 22, respectively; or
ii. SEQ ID NO: 302, SEQ ID NO: 303, and SEQ ID NO: 22, respectively; or
jj. SEQ ID NO: 304, SEQ ID NO: 305, and SEQ ID NO: 22, respectively; or
z. SEQ ID NO: 306, SEQ ID NO: 307, and SEQ ID NO: 22, respectively; or
aa. SEQ ID NO: 364, SEQ ID NO: 365, and SEQ ID NO: 22, respectively; or
bb. SEQ ID NO: 366, SEQ ID NO: 367, and SEQ ID NO: 22, respectively; or
cc. SEQ ID NO: 368, SEQ ID NO: 369, and SEQ ID NO: 22, respectively; or
dd. SEQ ID NO: 390, SEQ ID NO: 389, and SEQ ID NO: 10, respectively; or
ee. SEQ ID NO: 382, SEQ ID NO: 389, and SEQ ID NO: 10, respectively; or
ff. SEQ ID NO: 382, SEQ ID NO: 385, and SEQ ID NO: 10, respectively; or
gg. SEQ ID NO: 382, SEQ ID NO: 385, and SEQ ID NO: 11, respectively; or
hh. SEQ ID NO: 383, SEQ ID NO: 389, and SEQ ID NO: 10, respectively; or
ii. SEQ ID NO: 383, SEQ ID NO: 386, and SEQ ID NO: 10, respectively; or
jj. SEQ ID NO: 383, SEQ ID NO: 386, and SEQ ID NO: 12, respectively; or
kk. SEQ ID NO: 384, SEQ ID NO: 389, and SEQ ID NO: 10, respectively; or
ll. SEQ ID NO: 384, SEQ ID NO: 387, and SEQ ID NO: 10, respectively; or
mm. SEQ ID NO: 382, SEQ ID NO: 388, and SEQ ID NO: 10, respectively; or
nn. SEQ ID NO: 382, SEQ ID NO: 388, and SEQ ID NO: 11, respectively; or
oo. SEQ ID NO: 382, SEQ ID NO: 386, and SEQ ID NO: 10, respectively; or
pp. SEQ ID NO: 384, SEQ ID NO: 386, and SEQ ID NO: 10, respectively; or
qq. SEQ ID NO: 382, SEQ ID NO: 386, and SEQ ID NO: 12, respectively; or
rr. SEQ ID NO: 384, SEQ ID NO: 386, and SEQ ID NO: 12, respectively; or
ss. SEQ ID NO: 382, SEQ ID NO: 387, and SEQ ID NO: 10, respectively; or
tt. SEQ ID NO: 383, SEQ ID NO: 387, and SEQ ID NO: 10, respectively; or
uu. SEQ ID NO: 383, SEQ ID NO: 387, and SEQ ID NO: 11, respectively; or
vv. SEQ ID NO: 384, SEQ ID NO: 387, and SEQ ID NO: 11, respectively; or
ww. SEQ ID NO: 384, SEQ ID NO: 385, and SEQ ID NO: 11, respectively; or
xx. SEQ ID NO: 370, SEQ ID NO: 371, and SEQ ID NO: 10, respectively; or
yy. SEQ ID NO: 372, SEQ ID NO: 373, and SEQ ID NO: 10, respectively; or
zz. SEQ ID NO: 374, SEQ ID NO: 375, and SEQ ID NO: 10, respectively or
aaa. SEQ ID NO: 376, SEQ ID NO: 377, and SEQ ID NO: 10, respectively; or
bbb. SEQ ID NO: 378, SEQ ID NO: 379, and SEQ ID NO: 10, respectively; or
ccc. SEQ ID NO: 380, SEQ ID NO: 381, and SEQ ID NO: 10, respectively.

In some embodiments of the kit described herein, it further comprises at least one CRISPR associated protein (Cas protein) or a variant thereof, or at least one polynucleotide encoding the at least one Cas protein or a variant thereof, wherein the at least one Cas protein or a variant thereof are the same or different.

In an aspect, the present disclosure provides a gene editing system comprising a helper crRNA (hcrRNA) and a main crRNA (mcrRNA), or at least one DNA polynucleotide encoding the hcrRNA and/or the mcrRNA, wherein the hcrRNA comprises a first spacer sequence and a first linker sequence, wherein the first linker sequence comprises a first protein-binding motif, and the mcrRNA comprises a second spacer sequence and a second linker sequence, wherein the second linker sequence optionally comprises a second protein binding motif.

In some embodiments of the gene editing system described herein, the hcrRNA is the engineered crRNA described herein of any one of SEQ ID NOs: 1-3 and 149-151.

In some embodiments of the gene editing system described herein, the mcrRNA is the engineered crRNA described herein of any one of SEQ ID NOs: 4-7 and 152-153.

In some embodiments of the gene editing system described herein, the hcrRNA is the engineered crRNA described herein of any one of SEQ ID NOs: 1-3 and 149-151, and the mcrRNA is the engineered crRNA described herein of any one of SEQ ID NOs: 4-7 and 152-153.

In some embodiments of the gene editing system described herein, the first linker sequence and the second linker sequence are
a. SEQ ID NO: 1 and SEQ ID NO: 8, respectively; or
b. SEQ ID NO: 1 and SEQ ID NO: 4, respectively; or
c. SEQ ID NO: 2 and SEQ ID NO: 8, respectively; or
d. SEQ ID NO: 2 and SEQ ID NO: 5, respectively; or
e. SEQ ID NO: 3 and SEQ ID NO: 8, respectively; or
f. SEQ ID NO: 3 and SEQ ID NO: 6, respectively; or
g. SEQ ID NO: 1 and SEQ ID NO: 7, respectively; or
h. SEQ ID NO: 1 and SEQ ID NO: 5, respectively; or
i. SEQ ID NO: 3 and SEQ ID NO: 5, respectively; or
j. SEQ ID NO: 1 and SEQ ID NO: 6, respectively; or
k. SEQ ID NO: 2 and SEQ ID NO: 6, respectively; or
l. SEQ ID NO: 3 and SEQ ID NO: 6, respectively; or
m. SEQ ID NO: 3 and SEQ ID NO: 4, respectively; or
n. SEQ ID NO: 149 and SEQ ID NO: 152, respectively; or
o. SEQ ID NO: 150 and SEQ ID NO: 152, respectively; or
p. SEQ ID NO: 151 and SEQ ID NO: 152, respectively; or
q. SEQ ID NO: 149 and SEQ ID NO: 153, respectively; or
r. SEQ ID NO: 150 and SEQ ID NO: 153, respectively; or
s. SEQ ID NO: 151 and SEQ ID NO: 153, respectively.

In some embodiments of the gene editing system described herein, the hcrRNA and the mcrRNA are
a. SEQ ID NO: 21 and SEQ ID NO: 20, respectively; or
b. SEQ ID NO: 13 and SEQ ID NO: 20, respectively; or
c. SEQ ID NO: 13 and SEQ ID NO: 16, respectively; or
d. SEQ ID NO: 14 and SEQ ID NO: 20, respectively; or
e. SEQ ID NO: 14 and SEQ ID NO: 17, respectively; or
f. SEQ ID NO: 15 and SEQ ID NO: 20, respectively; or
g. SEQ ID NO: 15 and SEQ ID NO: 18, respectively; or
h. SEQ ID NO: 13 and SEQ ID NO: 19, respectively; or
i. SEQ ID NO: 13 and SEQ ID NO: 17, respectively; or
j. SEQ ID NO: 15 and SEQ ID NO: 17, respectively; or
k. SEQ ID NO: 13 and SEQ ID NO: 18, respectively; or
l. SEQ ID NO: 14 and SEQ ID NO: 18, respectively; or
m. SEQ ID NO: 15 and SEQ ID NO: 18, respectively; or
n. SEQ ID NO: 15 and SEQ ID NO: 16, respectively; or
o. SEQ ID NO: 154 and SEQ ID NO: 157, respectively; or
p. SEQ ID NO: 155 and SEQ ID NO: 157, respectively; or
q. SEQ ID NO: 156 and SEQ ID NO: 157, respectively; or
r. SEQ ID NO: 154 and SEQ ID NO: 158, respectively; or
s. SEQ ID NO: 155 and SEQ ID NO: 158, respectively; or
kk. SEQ ID NO: 156 and SEQ ID NO: 158, respectively; or
ll. SEQ ID NO: 302 and SEQ ID NO: 303, respectively; or
mm. SEQ ID NO: 304 and SEQ ID NO: 305, respectively; or
t. SEQ ID NO: 306 and SEQ ID NO: 307, respectively; or
u. SEQ ID NO: 364 and SEQ ID NO: 365, respectively; or
v. SEQ ID NO: 366 and SEQ ID NO: 367, respectively; or
w. SEQ ID NO: 368 and SEQ ID NO: 369, respectively; or
x. SEQ ID NO: 390, and SEQ ID NO: 389, respectively; or
y. SEQ ID NO: 382, and SEQ ID NO: 389, respectively; or
z. SEQ ID NO: 382, and SEQ ID NO: 385, respectively; or
aa. SEQ ID NO: 382, and SEQ ID NO: 385, respectively; or
bb. SEQ ID NO: 383, and SEQ ID NO: 389, respectively; or
cc. SEQ ID NO: 383, and SEQ ID NO: 386, respectively; or
dd. SEQ ID NO: 383, and SEQ ID NO: 386, respectively; or
ee. SEQ ID NO: 384, and SEQ ID NO: 389, respectively; or
ff. SEQ ID NO: 384, and SEQ ID NO: 387, respectively; or
gg. SEQ ID NO: 382, and SEQ ID NO: 388, respectively; or
hh. SEQ ID NO: 382, and SEQ ID NO: 388, respectively; or
ii. SEQ ID NO: 382, and SEQ ID NO: 386, respectively; or
jj. SEQ ID NO: 384, and SEQ ID NO: 386, respectively; or
kk. SEQ ID NO: 382, and SEQ ID NO: 386, respectively; or
ll. SEQ ID NO: 384, and SEQ ID NO: 386, respectively; or
mm. SEQ ID NO: 382, and SEQ ID NO: 387, respectively; or
nn. SEQ ID NO: 383, and SEQ ID NO: 387, respectively; or
oo. SEQ ID NO: 383, and SEQ ID NO: 387, respectively; or
pp. SEQ ID NO: 384, and SEQ ID NO: 387, respectively; or
qq. SEQ ID NO: 384, and SEQ ID NO: 385, respectively; or
rr. SEQ ID NO: 370 and SEQ ID NO: 371, respectively; or
ss. SEQ ID NO: 372 and SEQ ID NO: 373, respectively; or
tt. SEQ ID NO: 374 and SEQ ID NO: 375, respectively or
uu. SEQ ID NO: 376 and SEQ ID NO: 377, respectively; or
vv. SEQ ID NO: 378 and SEQ ID NO: 379, respectively; or
ww. SEQ ID NO: 380 and SEQ ID NO: 381, respectively.

In some embodiments of the gene editing system described herein, it further comprises a first tracrRNA and a second tracrRNA, wherein the first tracrRNA and second tracrRNA are the same or different.

In some embodiments of the gene editing system described herein, the first tracrRNA and the second tracrRNA each has a sequence of any one of SEQ ID NOs: 10-12. In some embodiments, the first and second tracrRNA is each selected from any one of SEQ ID NOs: 22-24.

In some embodiments of the gene editing system described herein, the first linker sequence, the second linker sequence, the first tracrRNA, and the second tracrRNA are
a. SEQ ID NO: 1, SEQ ID NO: 8, SEQ ID NO: 10, and SEQ ID NO: 10, respectively; or
b. SEQ ID NO: 1, SEQ ID NO: 4, SEQ ID NO: 10, and SEQ ID NO: 10, respectively; or
c. SEQ ID NO: 1, SEQ ID NO: 4, SEQ ID NO: 11, and SEQ ID NO: 11, respectively; or
d. SEQ ID NO: 2, SEQ ID NO: 8, SEQ ID NO: 10, and SEQ ID NO: 10, respectively; or
e. SEQ ID NO: 2, SEQ ID NO: 5, SEQ ID NO: 10, and SEQ ID NO: 10, respectively; or
f. SEQ ID NO: 2, SEQ ID NO: 5, SEQ ID NO: 12, and SEQ ID NO: 12, respectively; or
g. SEQ ID NO: 3, SEQ ID NO: 8, SEQ ID NO: 10, and SEQ ID NO: 10, respectively; or
h. SEQ ID NO: 3, SEQ ID NO: 6, SEQ ID NO: 10, and SEQ ID NO: 10, respectively; or
i. SEQ ID NO: 1, SEQ ID NO: 7, SEQ ID NO: 10, and SEQ ID NO: 10, respectively; or
j. SEQ ID NO: 1, SEQ ID NO: 7, SEQ ID NO: 11, and SEQ ID NO: 11, respectively; or
k. SEQ ID NO: 1, SEQ ID NO: 5, SEQ ID NO: 10, and SEQ ID NO: 10, respectively; or
l. SEQ ID NO: 3, SEQ ID NO: 5, SEQ ID NO: 10, and SEQ ID NO: 10, respectively; or
m. SEQ ID NO: 1, SEQ ID NO: 5, SEQ ID NO: 12, and SEQ ID NO: 12, respectively; or
n. SEQ ID NO: 3, SEQ ID NO: 5, SEQ ID NO: 12, and SEQ ID NO: 12, respectively; or
o. SEQ ID NO: 1, SEQ ID NO: 6, SEQ ID NO: 10, and SEQ ID NO: 10, respectively; or
p. SEQ ID NO: 2, SEQ ID NO: 6, SEQ ID NO: 10, and SEQ ID NO: 10, respectively; or
q. SEQ ID NO: 2, SEQ ID NO: 6, SEQ ID NO: 11, and SEQ ID NO: 11, respectively; or
r. SEQ ID NO: 3, SEQ ID NO: 6, SEQ ID NO: 11, and SEQ ID NO: 11, respectively; or
s. SEQ ID NO: 3, SEQ ID NO: 4, SEQ ID NO: 11, and SEQ ID NO: 11, respectively; or
t. SEQ ID NO: 149, SEQ ID NO: 152, SEQ ID NO: 10 and SEQ ID NO: 10, respectively; or
u. SEQ ID NO: 150, SEQ ID NO: 152, SEQ ID NO: 10 and SEQ ID NO: 10, respectively; or
v. SEQ ID NO: 151, SEQ ID NO: 152, SEQ ID NO: 10 and SEQ ID NO: 10, respectively; or
w. SEQ ID NO: 149, SEQ ID NO: 153, SEQ ID NO: 10 and SEQ ID NO: 10, respectively; or
x. SEQ ID NO: 150, SEQ ID NO: 153, SEQ ID NO: 10 and SEQ ID NO: 10, respectively; or
y. SEQ ID NO: 151, SEQ ID NO: 153, SEQ ID NO: 10 and SEQ ID NO: 10, respectively.

In some embodiments of the gene editing system described herein, the hcrRNA, the mcrRNA, the first tracrRNA, and the second tracrRNA are
a. SEQ ID NO: 21, SEQ ID NO: 20, SEQ ID NO: 22, and SEQ ID NO: 22, respectively; or
b. SEQ ID NO: 13, SEQ ID NO: 20, SEQ ID NO: 22, and SEQ ID NO: 22, respectively; or
c. SEQ ID NO: 13, SEQ ID NO: 16, SEQ ID NO: 22, and SEQ ID NO: 22, respectively; or
d. SEQ ID NO: 13, SEQ ID NO: 16, SEQ ID NO: 23, and SEQ ID NO: 23, respectively; or
e. SEQ ID NO: 14, SEQ ID NO: 20, SEQ ID NO: 22, and SEQ ID NO: 22, respectively; or
f. SEQ ID NO: 14, SEQ ID NO: 17, SEQ ID NO: 22, and SEQ ID NO: 22, respectively; or
g. SEQ ID NO: 14, SEQ ID NO: 17, SEQ ID NO: 24, and SEQ ID NO: 24, respectively; or
h. SEQ ID NO: 15, SEQ ID NO: 20, SEQ ID NO: 22, and SEQ ID NO: 22, respectively; or
i. SEQ ID NO: 15, SEQ ID NO: 18, SEQ ID NO: 22, and SEQ ID NO: 22, respectively; or
j. SEQ ID NO: 13, SEQ ID NO: 19, SEQ ID NO: 22, and SEQ ID NO: 22, respectively; or
k. SEQ ID NO: 13, SEQ ID NO: 19, SEQ ID NO: 23, and SEQ ID NO: 23, respectively; or
l. SEQ ID NO: 13, SEQ ID NO: 17, SEQ ID NO: 22, and SEQ ID NO: 22, respectively; or
m. SEQ ID NO: 15, SEQ ID NO: 17, SEQ ID NO: 22, and SEQ ID NO: 22, respectively; or
n. SEQ ID NO: 13, SEQ ID NO: 17, SEQ ID NO: 24, and SEQ ID NO: 24, respectively; or
o. SEQ ID NO: 15, SEQ ID NO: 17, SEQ ID NO: 24, and SEQ ID NO: 24, respectively; or
p. SEQ ID NO: 13, SEQ ID NO: 18, SEQ ID NO: 22, and SEQ ID NO: 22, respectively; or
q. SEQ ID NO: 14, SEQ ID NO: 18, SEQ ID NO: 22, and SEQ ID NO: 22, respectively; or
r. SEQ ID NO: 14, SEQ ID NO: 18, SEQ ID NO: 23, and SEQ ID NO: 23, respectively; or
s. SEQ ID NO: 15, SEQ ID NO: 18, SEQ ID NO: 23, and SEQ ID NO: 23, respectively; or
t. SEQ ID NO: 15, SEQ ID NO: 16, SEQ ID NO: 23, and SEQ ID NO: 23, respectively; or
u. SEQ ID NO: 154, SEQ ID NO: 157, SEQ ID NO: 22, and SEQ ID NO: 22, respectively; or
v. SEQ ID NO: 155, SEQ ID NO: 157, SEQ ID NO: 22, and SEQ ID NO: 22, respectively; or
w. SEQ ID NO: 156, SEQ ID NO: 157, SEQ ID NO: 22, and SEQ ID NO: 22, respectively; or
x. SEQ ID NO: 154, SEQ ID NO: 158, SEQ ID NO: 22, and SEQ ID NO: 22, respectively; or
y. SEQ ID NO: 155, SEQ ID NO: 158, SEQ ID NO: 22, and SEQ ID NO: 22, respectively; or
nn. SEQ ID NO: 156, SEQ ID NO: 158, SEQ ID NO: 22, and SEQ ID NO: 22, respectively; or
oo. SEQ ID NO: 302, SEQ ID NO: 303, SEQ ID NO: 22, and SEQ ID NO: 22, respectively; or
pp. SEQ ID NO: 304, SEQ ID NO: 305, SEQ ID NO: 22, and SEQ ID NO: 22, respectively; or
z. SEQ ID NO: 306, SEQ ID NO: 307, SEQ ID NO: 22, and SEQ ID NO: 22, respectively; or
aa. SEQ ID NO: 364, SEQ ID NO: 365, SEQ ID NO: 22, and SEQ ID NO: 22, respectively; or
bb. SEQ ID NO: 366, SEQ ID NO: 367, SEQ ID NO: 22, and SEQ ID NO: 22, respectively; or
cc. SEQ ID NO: 368, SEQ ID NO: 369, SEQ ID NO: 22, and SEQ ID NO: 22, respectively;
dd. SEQ ID NO: 390, SEQ ID NO: 389, SEQ ID NO: 10, and SEQ ID NO: 10, respectively; or
ee. SEQ ID NO: 382, SEQ ID NO: 389, SEQ ID NO: 10, and SEQ ID NO: 10, respectively; or
ff. SEQ ID NO: 382, SEQ ID NO: 385, SEQ ID NO: 10, and SEQ ID NO: 10, respectively; or
gg. SEQ ID NO: 382, SEQ ID NO: 385, SEQ ID NO: 11, and SEQ ID NO: 11, respectively; or
hh. SEQ ID NO: 383, SEQ ID NO: 389, SEQ ID NO: 10, and SEQ ID NO: 10, respectively; or
ii. SEQ ID NO: 383, SEQ ID NO: 386, SEQ ID NO: 10, and SEQ ID NO: 10, respectively; or
jj. SEQ ID NO: 383, SEQ ID NO: 386, SEQ ID NO: 12, and SEQ ID NO: 12, respectively; or
kk. SEQ ID NO: 384, SEQ ID NO: 389, SEQ ID NO: 10, and SEQ ID NO: 10, respectively; or
ll. SEQ ID NO: 384, SEQ ID NO: 387, SEQ ID NO: 10, and SEQ ID NO: 10, respectively; or
mm. SEQ ID NO: 382, SEQ ID NO: 388, SEQ ID NO: 10, and SEQ ID NO: 10, respectively; or
nn. SEQ ID NO: 382, SEQ ID NO: 388, SEQ ID NO: 11, and SEQ ID NO: 11, respectively; or
oo. SEQ ID NO: 382, SEQ ID NO: 386, SEQ ID NO: 10, and SEQ ID NO: 10, respectively; or
pp. SEQ ID NO: 384, SEQ ID NO: 386, SEQ ID NO: 10, and SEQ ID NO: 10, respectively; or
qq. SEQ ID NO: 382, SEQ ID NO: 386, SEQ ID NO: 12, and SEQ ID NO: 12, respectively; or
rr. SEQ ID NO: 384, SEQ ID NO: 386, SEQ ID NO: 12, and SEQ ID NO: 12, respectively; or
ss. SEQ ID NO: 382, SEQ ID NO: 387, SEQ ID NO: 10, and SEQ ID NO: 10, respectively; or
tt. SEQ ID NO: 383, SEQ ID NO: 387, SEQ ID NO: 10, and SEQ ID NO: 10, respectively; or
uu. SEQ ID NO: 383, SEQ ID NO: 387, SEQ ID NO: 11, and SEQ ID NO: 11, respectively; or
vv. SEQ ID NO: 384, SEQ ID NO: 387, SEQ ID NO: 11, and SEQ ID NO: 11, respectively; or
ww. SEQ ID NO: 384, SEQ ID NO: 385, SEQ ID NO: 11, and SEQ ID NO: 11, respectively; or
xx. SEQ ID NO: 370, SEQ ID NO: 371, SEQ ID NO: 10, and SEQ ID NO: 10, respectively; or
yy. SEQ ID NO: 372, SEQ ID NO: 373, SEQ ID NO: 10, and SEQ ID NO: 10, respectively; or
zz. SEQ ID NO: 374, SEQ ID NO: 375, SEQ ID NO: 10, and SEQ ID NO: 10, respectively or
aaa. SEQ ID NO: 376, SEQ ID NO: 377, SEQ ID NO: 10, and SEQ ID NO: 10, respectively; or
bbb. SEQ ID NO: 378, SEQ ID NO: 379, SEQ ID NO: 10, and SEQ ID NO: 10, respectively; or
ccc. SEQ ID NO: 380, SEQ ID NO: 381, SEQ ID NO: 10, and SEQ ID NO: 10, respectively.

In some embodiments of the gene editing system described herein, the gene editing system comprises
a. the hcrRNA comprising a first spacer sequence and a first linker sequence, wherein the first linker sequence comprises a first protein-binding motif,
b. the mcrRNA comprising a second spacer sequence and a second linker sequence, wherein the second linker sequence comprises a second protein-binding motif,
c. a first tracrRNA which is capable of forming a first base-pair structure with the hcrRNA,
d. a second tracrRNA which is capable of forming a second base-pair structure with the mcrRNA,
e. a first CRISPR-associated protein (Cas protein), or a polynucleotide encoding the first Cas protein, wherein the first Cas protein binds to the first base-pair structure,
f. a second Cas protein, or a polynucleotide encoding the second Cas protein, wherein the second Cas protein binds to the second base pair structure,
g. a first fusion protein comprising a nucleobase deaminase or a catalytic domain thereof and a first RNA binding domain, or a polynucleotide encoding the first fusion protein, wherein the nucleobase deaminase or the catalytic domain thereof and the first RNA binding domain are optionally connected by a linker, and wherein the first RNA binding domain binds to the first protein-binding motif,
wherein the first Cas protein and the second Cas protein are the same or different, and the first tracrRNA and the second tracrRNA are the same or different.

In some embodiments of the gene editing system described herein, it further comprises
a. a protease, or a polynucleotide encoding the protease, and
b. a nucleobase deaminase inhibitor domain,
wherein the nucleobase deaminase inhibitor domain is connected to the nucleobase deaminase or the catalytic domain thereof in the first fusion protein optionally by a linker, and wherein there is a cleavage site for the protease between the nucleobase deaminase inhibitor domain and the nucleobase deaminase or the catalytic domain thereof.

In some embodiments of the gene editing system described herein, the gene editing system comprises
a. the hcrRNA comprising a first spacer sequence and a first linker sequence, wherein the first linker sequence comprises a first protein-binding motif,
b. the mcrRNA comprising a second spacer sequence and a second linker sequence, wherein the second linker sequence comprises a second protein-binding motif,
c. a first tracrRNA which is capable of forming a first base-pair structure with the hcrRNA,
d. a second tracrRNA which is capable of forming a second base-pair structure with the mcrRNA,
e. a first CRISPR-associated protein (Cas protein), or a polynucleotide encoding the first Cas protein, wherein the first Cas protein binds to the first base-pair structure,
f. a second Cas protein, or a polynucleotide encoding the second Cas protein, wherein the second Cas protein binds to the second base pair structure,
g. a first fusion protein comprising a nucleobase deaminase or a catalytic domain thereof and a first RNA binding domain, or a polynucleotide encoding the first fusion protein, wherein the nucleobase deaminase or the catalytic domain thereof and the first RNA binding domain are optionally connected by a linker, and wherein the first RNA binding domain binds to the first protein-binding motif,
h. a protease, or a polynucleotide encoding the protease,
i. a nucleobase deaminase inhibitor domain, and
j. a second fusion protein comprising the protease and a second RNA binding domain, or a polynucleotide encoding the second fusion protein,
wherein the first Cas protein and the second Cas protein are the same or different, and the first tracrRNA and the second tracrRNA are the same or different,
wherein the nucleobase deaminase inhibitor domain is connected to the nucleobase deaminase or the catalytic domain thereof in the first fusion protein optionally by a linker, and wherein there is a cleavage site for the protease between the nucleobase deaminase inhibitor domain and the nucleobase deaminase or the catalytic domain thereof,
wherein the protease and the second RNA binding domain are optionally connected by a linker, and
wherein the second RNA binding domain binds to the second protein-binding motif.

In some embodiments of the gene editing system described herein, the protease is split into a first protease fragment and a second protease fragment, wherein the first and/or second protease fragment alone is not able to cleave the cleavage site.

In some embodiments of the gene editing system described herein, wherein the gene editing system comprises
a. the hcrRNA comprising a first spacer sequence and a first linker sequence, wherein the first linker sequence comprises a first protein-binding motif,
b. the mcrRNA comprising a second spacer sequence and a second linker sequence, wherein the second linker sequence comprises a second protein-binding motif,
c. a first tracrRNA which is capable of forming a first base-pair structure with the hcrRNA,
d. a second tracrRNA which is capable of forming a second base-pair structure with the mcrRNA,
e. a first CRISPR-associated protein (Cas protein), or a polynucleotide encoding the first Cas protein, wherein the first Cas protein binds to the first base-pair structure,
f. a second Cas protein, or a polynucleotide encoding the second Cas protein, wherein the second Cas protein binds to the second base pair structure,
g. a first fusion protein comprising a nucleobase deaminase or a catalytic domain thereof and a first RNA binding domain, or a polynucleotide encoding the first fusion protein, wherein the nucleobase deaminase or the catalytic domain thereof and the first RNA binding domain are optionally connected by a linker, and wherein the first RNA binding domain binds to the first protein-binding motif,
h. a protease, or a polynucleotide encoding the protease, wherein the protease is split into a first protease fragment and a second protease fragment, wherein the first and/or second protease fragment alone is not able to cleave the cleavage site,
i. a nucleobase deaminase inhibitor domain,
j. a second fusion protein comprising the first protease fragment and a second RNA binding domain, or a polynucleotide encoding the second fusion protein, wherein the first protease fragment and the second RNA binding domain are optionally connected by a linker, and
k. a third fusion protein comprising the second protease fragment and a third RNA binding domain, or a polynucleotide encoding the third fusion protein, wherein the second protease fragment and the third RNA binding domain are optionally connected by a linker,
wherein the first Cas protein and the second Cas protein are the same or different, and the first tracrRNA and the second tracrRNA are the same or different,
wherein the nucleobase deaminase inhibitor domain is connected to the nucleobase deaminase or the catalytic domain thereof in the first fusion protein optionally by a linker, and wherein there is a cleavage site for the protease between the nucleobase deaminase inhibitor domain and the nucleobase deaminase or the catalytic domain thereof,
wherein the mcrRNA further comprises a third protein-binding motif,
wherein the second RNA binding domain binds to the second protein-binding motif, and
wherein the third RNA binding domain binds to the third protein-binding motif.

In some embodiments of the gene editing system described herein, the gene editing system comprises
a. the hcrRNA comprising a first spacer sequence and a first linker sequence, wherein the first linker sequence comprises a first protein-binding motif,
b. the mcrRNA comprising a second spacer sequence and a second linker sequence, wherein the second linker sequence comprises a second protein-binding motif,
c. a first tracrRNA which is capable of forming a first base-pair structure with the hcrRNA,
d. a second tracrRNA which is capable of forming a second base-pair structure with the mcrRNA,
e. a first CRISPR-associated protein (Cas protein), or a polynucleotide encoding the first Cas protein, wherein the first Cas protein binds to the first base-pair structure,
f. a second Cas protein, or a polynucleotide encoding the second Cas protein, wherein the second Cas protein binds to the second base pair structure,
g. a first fusion protein comprising a nucleobase deaminase or a catalytic domain thereof and a first RNA binding domain, or a polynucleotide encoding the first fusion protein, wherein the nucleobase deaminase or the catalytic domain thereof and the first RNA binding domain are optionally connected by a linker, and wherein the first RNA binding domain binds to the first protein-binding motif,
h. a protease, or a polynucleotide encoding the protease, wherein the protease is split into a first protease fragment and a second protease fragment, wherein the first and/or second protease fragment alone is not able to cleave the cleavage site,
i. a nucleobase deaminase inhibitor domain,
j. a second fusion protein comprising the first protease fragment and a second RNA binding domain, or a polynucleotide encoding the second fusion protein, wherein the first protease fragment and the second RNA binding domain are optionally connected by a linker, and
k. a third fusion protein comprising the second protease fragment and a third RNA binding domain, or a polynucleotide encoding the third fusion protein, wherein the second protease fragment and the third RNA binding domain are optionally connected by a linker,
wherein the first Cas protein and the second Cas protein are the same or different, and the first tracrRNA and the second tracrRNA are the same or different,
wherein the nucleobase deaminase inhibitor domain is connected to the nucleobase deaminase or the catalytic domain thereof in the first fusion protein optionally by a linker, and wherein there is a cleavage site for the protease between the nucleobase deaminase inhibitor domain and the nucleobase deaminase or the catalytic domain thereof,
wherein the mcrRNA further comprises a third protein-binding motif,
wherein the second RNA binding domain binds to the second protein-binding motif,
wherein the third RNA binding domain binds to the third protein-binding motif, and
wherein the second and the third RNA binding domains are the same or different, and the second and the third protein-binding motifs are the same or different.

In some embodiments of the gene editing system described herein, the gene editing system comprises
a. the hcrRNA comprising a first spacer sequence and a first linker sequence, wherein the first linker sequence comprises a first protein-binding motif,
b. the mcrRNA comprising a second spacer sequence and a second linker sequence, wherein the second linker sequence comprises a second protein-binding motif,
c. a first tracrRNA which is capable of forming a first base-pair structure with the hcrRNA,
d. a second tracrRNA which is capable of forming a second base-pair structure with the mcrRNA,
e. a first CRISPR-associated protein (Cas protein), or a polynucleotide encoding the first Cas protein, wherein the first Cas protein binds to the first base-pair structure,
f. a second Cas protein, or a polynucleotide encoding the second Cas protein, wherein the second Cas protein binds to the second base pair structure,
g. a first fusion protein comprising a nucleobase deaminase or a catalytic domain thereof and a first RNA binding domain, or a polynucleotide encoding the first fusion protein, wherein the nucleobase deaminase or the catalytic domain thereof and the first RNA binding domain are optionally connected by a linker, and wherein the first RNA binding domain binds to the first protein-binding motif,
h. a protease, or a polynucleotide encoding the protease, wherein the protease is split into a first protease fragment and a second protease fragment, wherein the first and/or second protease fragment alone is not able to cleave the cleavage site,
i. a nucleobase deaminase inhibitor domain,
j. a second fusion protein comprising the first protease fragment and a second RNA binding domain, or a polynucleotide encoding the second fusion protein,
wherein the first Cas protein and the second Cas protein are the same or different, and the first tracrRNA and the second tracrRNA are the same or different,
wherein the nucleobase deaminase inhibitor domain is connected to the nucleobase deaminase or the catalytic domain thereof in the first fusion protein optionally by a linker, and wherein there is a cleavage site for the protease between the nucleobase deaminase inhibitor domain and the nucleobase deaminase or the catalytic domain thereof,
wherein the first protease fragment and the second RNA binding domain are optionally connected by a linker, and
wherein the second RNA binding domain binds to the second protein-binding motif.

In some embodiments of the gene editing system described herein, the protease is a TEV protease, a TuMV protease, a PPV protease, a PVY protease, a ZIKV protease, or a WNV protease.

In some embodiments of the gene editing system described herein, the protease is a TEV protease comprising a sequence of SEQ ID NO: 25.

In some embodiments of the gene editing system described herein, the first TEV protease fragment comprises a sequence of SEQ ID NO: 26 or SEQ ID NO: 27.

In some embodiments of the gene editing system described herein, the nucleobase deaminase inhibitor is an inhibitory domain of a nucleobase deaminase.

In some embodiments of the gene editing system described herein, the nucleobase deaminase inhibitor is an inhibitory domain of a cytidine deaminase and/or an adenosine deaminase.

In some embodiments of the gene editing system described herein, the inhibitory domain comprises an amino acid sequence of SEQ ID NO: 42-43 and 51-138.

In some embodiments of the gene editing system described herein, the nucleotide deaminase is a cytidine deaminase.

In some embodiments of the gene editing system described herein, the cytidine deaminase is selected from the group consisting of APOBEC3A (A3A), APOBEC3B (A3B), APOBEC3C (A3C), APOBEC3D (A3D), APOBEC3F (A3F), APOBEC3G (A3G), APOBEC3H (A3H), APOBEC1 (A1), APOBEC3 (A3), APOBEC2 (A2), APOBEC4 (A4), and AICDA (AID).

In some embodiments of the gene editing system described herein, the cytidine deaminase comprises an amino acid sequence of SEQ ID NO: 252-287.

In some embodiments of the gene editing system described herein, the cytidine deaminase is a naturally occurring cytidine deaminase, an engineered cytidine deaminase, an evolved cytidine deaminase, or an adenosine deaminase that possesses cytidine deaminase activity.

In some embodiments of the gene editing system described herein, the cytidine deaminase is a human or mouse cytidine deaminase.

In some embodiments of the gene editing system described herein, the catalytic domain of the cytidine deaminase is a mouse A3 cytidine deaminase domain 1 (mA3-CDA1) or human A3B cytidine deaminase domain 2 (hA3B-CDA2).

In some embodiments of the gene editing system described herein, the nucleotide deaminase is an adenosine deaminase.

In some embodiments of the gene editing system described herein, the adenosine deaminase is selected from the group consisting of tRNA-specific adenosine deaminase (TadA), adenosine deaminase tRNA specific 1 (ADAT1), adenosine deaminase tRNA specific 2 (ADAT2), adenosine deaminase tRNA specific 3 (ADAT3), adenosine deaminase RNA specific B1 (ADARB1), adenosine deaminase RNA specific B2 (ADARB2), adenosine monophosphate deaminase 1 (AMPD1), adenosine monophosphate deaminase 2 (AMPD2), adenosine monophosphate deaminase 3 (AMPD3), adenosine deaminase (ADA), adenosine deaminase 2 (ADA2), adenosine deaminase like (ADAL), adenosine deaminase domain containing 1 (ADAD1), adenosine deaminase domain containing 2 (ADAD2), and adenosine deaminase RNA specific (ADAR).

In some embodiments of the gene editing system described herein, the adenosine deaminase comprises an amino acid sequence of SEQ ID NO: 159-251.

In some embodiments of the gene editing system described herein, the adenosine deaminase is a naturally occurring adenosine deaminase, an engineered adenosine deaminase, an evolved adenosine deaminase, or a cytidine deaminase that possesses adenosine deaminase activity.

In some embodiments of the gene editing system described herein, the adenosine deaminase is a human or mouse adenosine deaminase.

In some embodiments of the gene editing system described herein, the first fusion protein comprises one or more nucleotide deaminase, and the one or more nucleotide deaminase are the same or different.

In some embodiments of the gene editing system described herein, each of the one or more nucleotide deaminase is a cytidine deaminase or an adenosine deaminase.

In some embodiments of the gene editing system described herein, the nucleotide deaminase is a fusion of at least one cytidine deaminase and at least one adenosine deaminase.

In some embodiments of the gene editing system described herein, the first fusion protein further comprises one or more copies of uracil glycosylase inhibitor (UGI).

In some embodiments of the gene editing system described herein, each of the Cas protein is a Cas9, a dead Cas9 (dCas9), or a Cas9 nickase (nCas9) selected from the group consisting of SpCas9, FnCas9, St1Cas9, St3Cas9, NmCas9, SaCas9, AsCpf1, LbCpf1, FnCpf1, VQR Cas9, EQR Cas9, VRER Cas9, Cas9-NG, xCas9, eCas9, SpCas9-HF1, HypaCas9, HiFiCas9, sniper-Cas9, SpG, SpRY, KKH SaCas9, CjCas9, Cas9-NRRH, Cas9-NRCH, Cas9-NRTH, SsCpf1, PcCpf1, BpCpf1, LiCpf1, PmCpf1, Lb2Cpf1, PbCpf1, PbCpf1, PeCpf1, PdCpf1, MbCpf1, EeCpf1, CmtCpf1, BsCpf1, BhCasl2b, AkCasl2b, BsCasl2b, AmCasl2b, AaCasl2b, RfxCasl3d, LwaCasl3a, PspCasl3b, PguCasl3b, and RanCasl3b.

In some embodiments of the gene editing system described herein, at least one of the tracrRNA is selected from SEQ ID Nos: 10-12. In some embodiments, the tracrRNA is any one of SEQ ID Nos: 22-24.

In some embodiments of the gene editing system described herein, the first protein-binding RNA motif and the first RNA binding domain, the second protein-binding RNA motif and the second RNA binding domain, and the third protein-binding RNA motif and the third RNA binding domain, are each independently selected from the group consisting of a MS2 phage operator stem-loop and MS2 coat protein (MCP) or an RNA-binding section thereof, a boxB and N22p or an RNA-binding section thereof, a telomerase Ku binding motif and Ku protein or an RNA-binding section thereof, a telomerase Sm7 binding motif and Sm7 protein or an RNA-binding section thereof, a PP7 phage operator stem-loop and PP7 coat protein (PCP) or an RNA-binding section thereof, a SfMu phage Com stem-loop and Com RNA binding protein or an RNA-binding section thereof, and an RNA aptamer and corresponding aptamer ligand or an RNA-binding section thereof.

In an aspect, the present disclosure provides a polynucleotide comprising a sequence encoding the engineered crRNA described herein. In another aspect, the present disclosure provides a polynucleotide comprising a sequence encoding the engineered tracrRNA described herein.

In an aspect, the present disclosure provides a polynucleotide comprising a sequence encoding all components except the first and second Cas proteins in the gene editing system described herein.

In an aspect, the present disclosure provides a kit comprising the polynucleotide which comprises a sequence encoding all components except the first and second Cas proteins in the gene editing system described herein, and a polynucleotide encoding the first and/or the second Cas protein in any one of the gene editing systems described herein.

In an aspect, the present disclosure provides a vector comprising the polynucleotide described herein.

In an aspect, the present disclosure provides a vector comprising the polynucleotide described herein.

In some embodiments of the vector described herein, the vector is a plasmid or a viral vector.

In some embodiments of the vector described herein, the vector is a polycistronic vector.

In an aspect, the present disclosure provides a kit comprising the vector described herein, and a vector comprising the polynucleotide encoding the first and/or second Cas protein in any one of the gene editing systems described herein.

In an aspect, the present disclosure provides a cell comprising the engineered crRNA described herein.

In an aspect, the present disclosure provides a cell comprising the gene editing system described herein.

In an aspect, the present disclosure provides a cell comprising the polynucleotide described herein.

In some embodiments of the cell in claim 61, further comprising a polynucleotide encoding the first and/or the second Cas protein described herein.

In an aspect, the present disclosure provides a cell comprising the vector described herein.

In some embodiments of the cell described herein, the cell comprises a vector described herein, and a vector comprising a polynucleotide encoding the first and/or the second Cas protein in the gene editing system described herein.

In some embodiments of the cell described herein, wherein the cell is a stem cell, a somatic cell, a blood cell, or an immune cell.

In some embodiments of the cell described herein, wherein the cell is a primary cell or a differentiated cell.

In some embodiments of the cell described herein, wherein the cell is a human cell.

In another aspect, the present disclosure provides a method for reducing low-density lipoprotein cholesterol (LDL-C) in a subject by editing the PCSK9 gene in the subject, comprising administering to the subject the gene editing system disclosed herein, wherein the hcrRNA and the mcrRNA are SEQ ID NO: 302 and SEQ ID NO: 303, respectively; or SEQ ID NO: 304 and SEQ ID NO: 305, respectively; or SEQ ID NO: 306 and SEQ ID NO: 307, respectively; or SEQ ID NO: 370 and SEQ ID NO: 371, respectively; or SEQ ID NO: 372 and SEQ ID NO: 373, respectively; or SEQ ID NO: 374 and SEQ ID NO: 375, respectively.

In another aspect, the present disclosure provides a method for reducing low-density lipoprotein cholesterol (LDL-C) and triglyceride in a subject by editing the ANGPTL3 gene in the subject, comprising administering to the subject the gene editing system disclosed herein, wherein the hcrRNA and the mcrRNA are SEQ ID NO: 364 and SEQ ID NO: 365, respectively; or SEQ ID NO: 366 and SEQ ID NO: 367, respectively; or SEQ ID NO: 368 and SEQ ID NO: 369, respectively; or SEQ ID NO: 376 and SEQ ID NO: 377, respectively; or SEQ ID NO: 378 and SEQ ID NO: 379, respectively; or SEQ ID NO: 380 and SEQ ID NO: 381, respectively.

### BRIEF DESCRIPTION OF THE FIGURES

FIG. 1 schematically illustrates various LigoRNA-based gene editing systems. FIG. 1(A) shows polynucleotide constructs encoding components of six versions of the LigoRNA-based gene editing system that comprises two LigoRNA structures (denoted as V1, V2, V3, V4, V5, and V6). FIG. 1(B,C) are illustrations of the six versions of the LigoRNA-based gene editing system (denoted as V1, V2, V3, V4, V5, and V6). FIG. 1(D) is an illustration of different variants of the V5 gene editing system, wherein the gene editing system comprises one or more nucleotide deaminases. Such variants can be similarly applied to other versions (e.g., V1, V2, V3, V4 and V6) of the LigoRNA-based gene editing system. FIG. 1(E) is an illustration of another embodiment of the LigoRNA-based gene editing system, which comprises one LigoRNA structure.

FIG. 2 schematically illustrates the original types (Combination 1) and a hairpin fused types of crRNA and tracrRNA (Combination 2). For brevity, names of certain RNAs in FIGS. 2-14 are shortened to not include the letters “RNA.” For example, “hcr-O” is the same as “hcrRNA-O,” which is SEQ ID NO: 9. For another example, “tracr-O,3” is the same as “tracrRNA-O,3,” which is SEQ ID NO: 10.

FIG. 3 schematically illustrates various hairpin fused types of crRNA and tracrRNA (Combination 3 and 4).

FIG. 4 schematically illustrates various hairpin fused types of crRNA and tracrRNA (Combination 5 and 6).

FIG. 5 schematically illustrates various hairpin fused types of crRNA and tracrRNA (Combination 7 and 8).

FIG. 6 schematically illustrates various hairpin fused types of crRNA and tracrRNA (Combination 9 and 10).

FIG. 7 schematically illustrates various hairpin fused types of crRNA and tracrRNA (Combination 11 and 12).

FIG. 8 schematically illustrates various hairpin fused types of crRNA and tracrRNA (Combination 13 and 14).

FIG. 9 schematically illustrates various hairpin fused types of crRNA and tracrRNA (Combination 15 and 16).

FIG. 10 schematically illustrates various hairpin fused types of crRNA and tracrRNA (Combination 17 and 18).

FIG. 11 schematically illustrates various hairpin fused types of crRNA and tracrRNA (Combination 19 and 20).

FIG. 12 schematically illustrates various hairpin fused types of crRNA and tracrRNA (Combination 21 and 22).

FIG. 13 schematically illustrates various hairpin fused types of crRNA and tracrRNA (Combination 23 and 24).

FIG. 14 schematically illustrates various hairpin fused types of crRNA and tracrRNA (Combination 25 and 26).

FIG. 15 shows C-to-T/G/A editing efficiencies induced by the V5-LigoRNA-based gene editing system with 11 different combinations of hcr-tracrRNA and mcr-tracrRNA structure in HSPC. In FIGS. 15-20, the target gene of the LigoRNA-based gene editing system is the HBG gene (the gamma globin gene). Certain combinations of hcr-tracrRNA and mcr-tracrRNA are illustrated in FIGS. 15-20. Components 1 and 2 in FIGS. 15-20 are illustrated in FIG. 1(B, C, D), wherein L refers to “Locator” and EK refers to “Effector & Key.” For brevity, names of certain RNAs in FIGS. 15-20 are shortened to not include the letters “HBG” and “RNA.” For example, “hcr-O” in FIGS. 15-20 is the same as “HBG-hcrRNA-O,” which is SEQ ID NO: 21.

FIG. 16 shows C-to-T/G/A editing efficiencies induced by the V5-LigoRNA-based gene editing system (LigoRNA-tCBE-V5) with 9 different combinations of hcr-tracrRNA and mcr-tracrRNA structure in HSPC. Edit frequencies were measured with cells cultured 48 h after electroporation.

FIG. 17 shows C-to-T/G/A editing efficiencies induced by the V5-LigoRNA-based gene editing system (LigoRNA-tCBE-V5) with 4 different combinations of hcr-tracrRNA and mcr-tracrRNA structure in HSPC. Edit frequencies were measured with cells cultured 48 h after electroporation.

FIG. 18 shows C-to-T/G/A editing efficiencies induced by the original V5-LigoRNA-based gene editing system (LigoRNA-tCBE-V5) or V6-LigoRNA-based gene editing system (LigoRNA-tCBE-V6) with 3 different combinations of hcr-tracrRNA and mcr-tracrRNA structure in HSPC. Edit frequencies were measured with cells cultured 48 h after electroporation.

FIG. 19 shows C-to-T/G/A editing efficiencies induced by the V5-LigoRNA-based gene editing system (LigoRNA-tCBE-V5) with 7 different combinations of hcr-tracrRNA and mcr-tracrRNA structure in HSPC. Edit frequencies were measured with cells cultured 48 h after electroporation.

FIG. 20 shows C-to-T/G/A editing efficiencies induced by the V5-LigoRNA-based gene editing system (LigoRNA-tCBE-V5) with combination 21 of hcr-tracrRNA and mcr-tracrRNA structure at different input level in HSPC. Edit frequencies were measured with cells cultured 48 h after electroporation.

FIG. 21 illustrates LNP delivery of LigoRNA-tCBE-V5 with combination 21 for in vivo base editing. FIG. 21 shows in vivo editing frequencies induced by LNP containing tBE system with end modified guide RNA or a LigoRNA-tCBE-V5 system, and the levels of plasma PCSK9 protein and cholesterol levels in the mice injected with LNP expressing tBE or LigoRNA-tCBE-V5.

FIG. 22 illustrates dual editing strategy by LigoRNA-tCBE-V5 with combination 21 in different cells (HepG2, Hepa1-6, and COS-1 cell). FIG. 22A shows editing frequency of co-editing by two pairs of mgRNA and hgRNA, wherein one pair target PCSK9 and the other pair target ANGPTL3. The editing frequency of co-editing is compared with their respective single editing control. FIG. 22B shows the dual editing strategy produced comparable protein level reduction compared to single editing as evidenced by ELISA assay.

### DETAILED DESCRIPTION

### Definitions

In the present disclosure, unless otherwise specified, the scientific and technical terms used herein have the meanings generally understood by a person skilled in the art. Although any methods and materials similar or equivalent to those described herein find use in the practice of the present disclosure, the preferred methods and materials are described herein. Accordingly, the terms defined herein are more fully described by reference to the Specification as a whole.

All publications, including but not limited to disclosures and disclosure applications, cited in this specification are herein incorporated by reference as though fully set forth. If certain content of a publication cited herein contradicts or is inconsistent with the present disclosure, the present disclosure controls.

As used herein, the singular terms “a,” “an,” and “the” include the plural reference unless the context clearly indicates otherwise.

As used herein, “and/or” refers to and encompasses any and all possible combinations of one or more of the associated listed items, as well as the lack of combinations when interpreted in the alternative (“or”). Moreover, the present invention also contemplates that in some embodiments of the invention, any feature or combination of features set forth herein can be excluded or omitted.

Unless the context requires otherwise, the terms “comprise,” “comprises,” and “comprising,” or similar terms are intended to mean a non-exclusive inclusion, such that a recited list of elements or features does not include those stated or listed elements solely but may include other elements or features that are not listed or stated.

Unless otherwise indicated, nucleic acids are written left to right in the 5′ to 3′ orientation, and amino acid sequences are written left to right in amino to carboxy orientation, respectively.

It is to be understood that this disclosure is not limited to the particular methodology, protocols, and reagents described, as these may vary, depending upon the context in which they are used by those skilled in the art.

As used herein, the terms “percent identity” and “% identity,” as applied to nucleic acid or polynucleotide sequences, refer to the percentage of residue matches between at least two nucleic acid or polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences.

Percent identity between nucleic acid or polynucleotide sequences may be determined using a suite of commonly used and freely available sequence comparison algorithms provided by the National Center for Biotechnology Information (NCBI) Basic Local Alignment Search Tool (BLAST) (Altschul, S. F. et al. (1990) J. Mol. Biol. 215:403-410), which is available from several sources, including the NCBI, Bethesda, Md., and on the Internet at http://www.ncbi.nlmr.nih.gov/BLAST/.

Nucleic acid or polynucleotide sequences that do not show a high degree of identity may nevertheless encode similar amino acid sequences due to the degeneracy of the genetic code. It is understood that changes in a nucleic acid sequence can be made using this degeneracy to produce multiple nucleic acid sequences that all encode substantially the same protein. Specifically, degenerate codon substitutions may be achieved by generating sequences in which the third position of one or more selected (or all) codons is substituted with mixed-base and/or deoxyinosine residues (Batzer et al. (1991) Nucleic Acid Res 19:5081; Ohtsuka et al. (1985) J Biol Chem 260:2605-2608; Cassol et al. (1992); Rossolini et al. (1994) Mol Cell Probes 8:91-98). The term “nucleic acid” refers to deoxyribonucleotides or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. The term nucleic acid is used interchangeably with polynucleotide, and (in appropriate contexts) gene, cDNA, and mRNA encoded by a gene.

As used herein, “percent (%) amino acid sequence identity” with respect to a peptide, polypeptide or protein sequence is defined as the percentage of amino acid residues in a candidate sequence that are identical with the amino acid residues in another peptide or polypeptide sequence, after aligning the sequences and introducing gaps, if necessary, to achieve the maximum percent sequence identity, and not considering any conservative substitutions as part of the sequence identity. Percent amino acid sequence identity in the current disclosure is measured using BLAST software. Those skilled in the art can determine appropriate parameters for measuring alignment, including any algorithms needed to achieve maximal alignment over the full length of the sequences being compared.

An amino acid substitution refers to the replacement of one amino acid in a polypeptide with another amino acid. Amino acid substitutions can be conservative or non-conservative substitutions. A conservative replacement (also called a conservative mutation or a conservative substitution) is an amino acid replacement in a protein that changes a given amino acid to a different amino acid with similar biochemical properties (e.g., charge, hydrophobicity, and size). Exemplary substitutions are shown in Table 1. Amino acid substitutions may be introduced into a protein of interest and the products screened for a desired activity, for example, retained/improved biological activity.

TABLE 1
Exemplary Substitutions

| Original Residue | Exemplary Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, “peptides,” “protein”, or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis.

As used herein, the term “encode” or “encoding” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

As used herein, a “single guide RNA” (sgRNA) refers to a synthetic or expressed RNA sequence that comprises a CRISPR binding motif and a spacer. A “spacer” is a DNA-targeting motif, which is a sequence that is complementary to a target specific DNA region. The CRISPR binding motif of a guide RNA can bind to a Cas enzyme and DNA-targeting motif of the gRNA can guide the complex to a specific target location on a DNA. A guide RNA may further comprise one or more protein-binding motifs.

As used herein, a CRISPR RNA (crRNA) refers to a synthetic or expressed RNA sequence that can form a base-paired structure with a trans-activating crRNA (tracrRNA), to which a Cas protein can bind and form an effector complex. The crRNA also comprises a spacer sequence, which is complementary to a target specific DNA region.

As used herein, a linker sequence in the context of crRNA refers to a region in the crRNA that is capable of forming a dual-RNA structure with another RNA sequence (such as a tracrRNA). In some embodiments, the linker sequence is at the 3′-end of the spacer sequence of the crRNA.

As used herein, a trans-activating crRNA (tracrRNA) refers to a synthetic or expressed RNA sequence that can form a base-paired structure with a crRNA, to which a Cas protein can bind and form an effector complex.

As used herein, a base-paired structure refers to a structure formed by two nucleic acid sequences, wherein the two nucleic acid sequences bind to each other through multiple Watson-Crick-Franklin base pairs formed between nucleotides. When the two nucleic acid sequences are RNA sequences, base pair is formed between guanine-cytosine and adenine-uracil.

As used herein, a “fusion protein” is a protein comprising at least two domains that are encoded by separate genes that have been joined a single polypeptide. For example, a fusion protein can comprise two domains that are encoded by separate genes that have been joined so that they are transcribed and translated as a single unit, producing a single polypeptide. In some embodiments, the at least two domains are fused together directly. In some embodiments, the domains are connected by one or more linkers.

As used herein, a “protein-binding RNA motif” refers to a piece of sequence in an RNA molecule that is capable of binding to proteins. In some embodiments, the protein-binding RNA motif is capable of binding to specific protein with high affinity and specificity. In some embodiments, the protein-binding RNA motif is an RNA aptamer or a variant thereof.

As used herein, a “RNA-binding domain” refers to a domain in a protein that is capable of binding to an RNA or a subpart of the RNA molecule. In some embodiments, the RNA-binding domain is a domain recognized and bound by an RNA aptamer or a variant thereof. In some embodiments, the RNA-binding domain is an RNA-recognition motif, an hnRNP K homology domain, or a DEAD box helicase domain.

The term “genetic modification” and its grammatical equivalents as used herein can refer to one or more alterations of a nucleic acid, e.g., the nucleic acid within an organism's genome. For example, genetic modification can refer to alterations, additions, and/or deletion of genes or portions of genes or other nucleic acid sequences. A genetically modified cell can also refer to a cell with an added, deleted, and/or altered gene or portion of a gene. A genetically modified cell can also refer to a cell with an added nucleic acid sequence that is not a gene or gene portion. Genetic modifications include, for example, both transient knock-in or knock-down mechanisms, and mechanisms that result in permanent knock-in, knock-down, or knock-out of target genes or portions of genes or nucleic acid sequences. Genetic modifications include, for example, both transient knock-in and mechanisms that result in permanent knock-in of nucleic acids sequences. Genetic modifications also include, for example, reduced or increased transcription, reduced or increased mRNA stability, reduced or increased translation, and reduced or increased protein stability.

As used herein, a composition refers to any mixture of two or more products, substances, or compounds, including cells.

### LigoRNA System

The present disclosure provides a novel LigoRNA system with a dual-RNA structure, which can be used as guide RNA in CRISPR-based gene editing systems. The dual-RNA structure can be formed by a ligand-bound CRISPR RNA (crRNA) and a trans-activating crRNA (tracrRNA). For example, the LigoRNA system comprises an hgRNA set of a hcrRNA and a tracrRNA, and an mgRNA set of mcrRNA and a tracrRNA. Preferably, all of these RNA molecules are not longer than 100 nucleotides.

Since the LigoRNA system is formed by two short RNAs, it helps to solve the problem of synthesizing long single guide RNAs in previous gene editing systems. Chemically synthesized RNAs over 100 nt demonstrated much lower yield and purity, resulting in challenges for large-scale production and cost control.

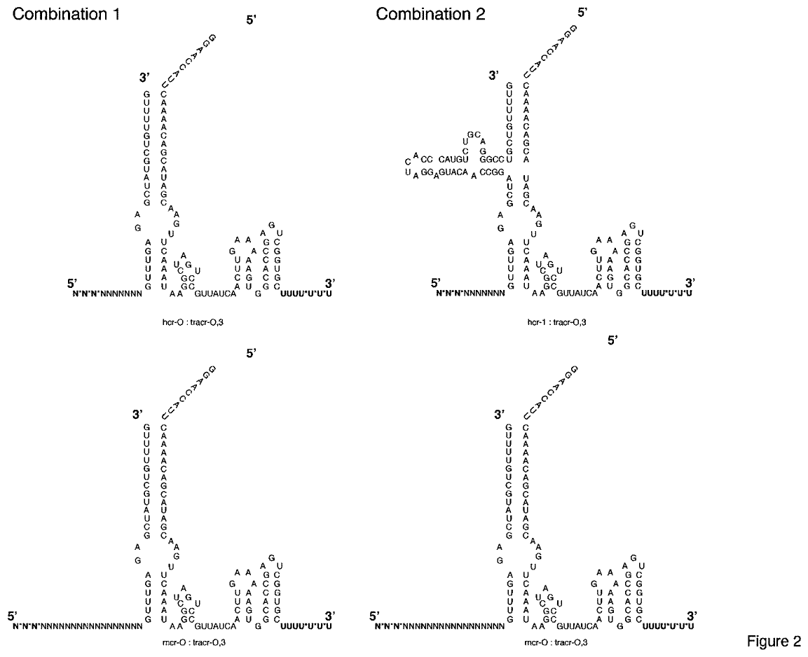

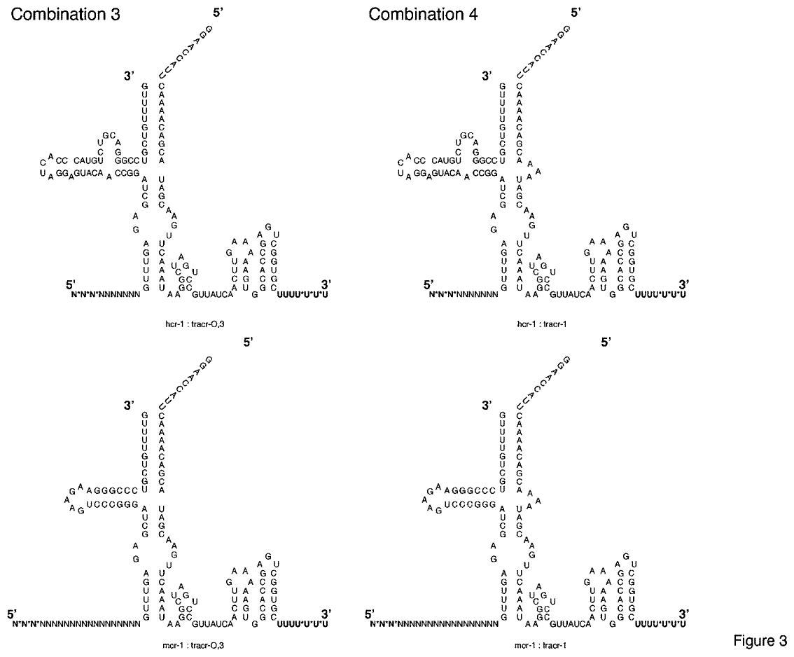

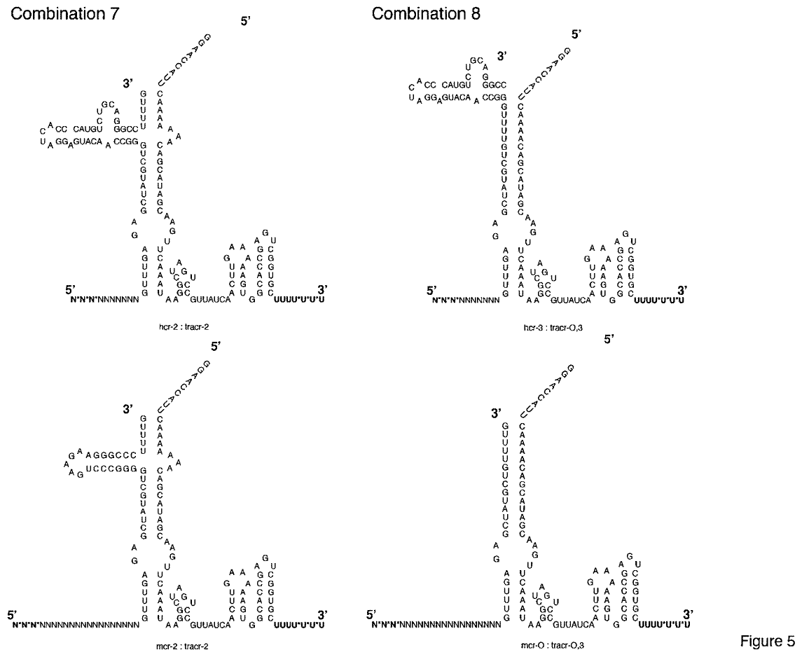

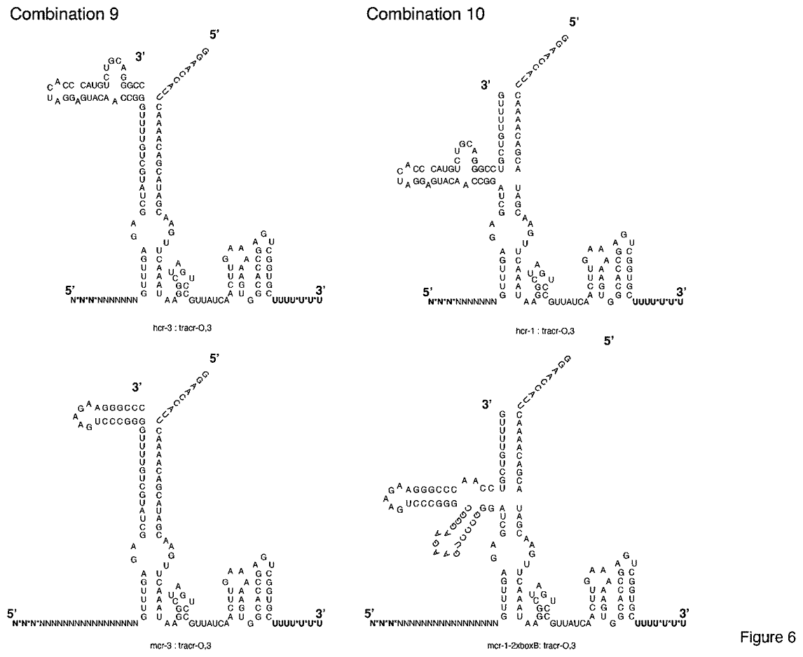

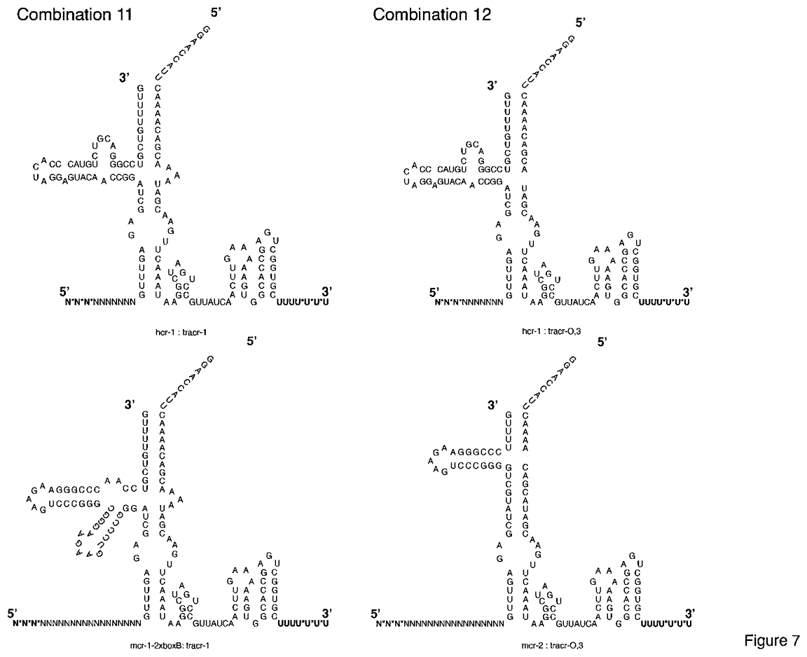

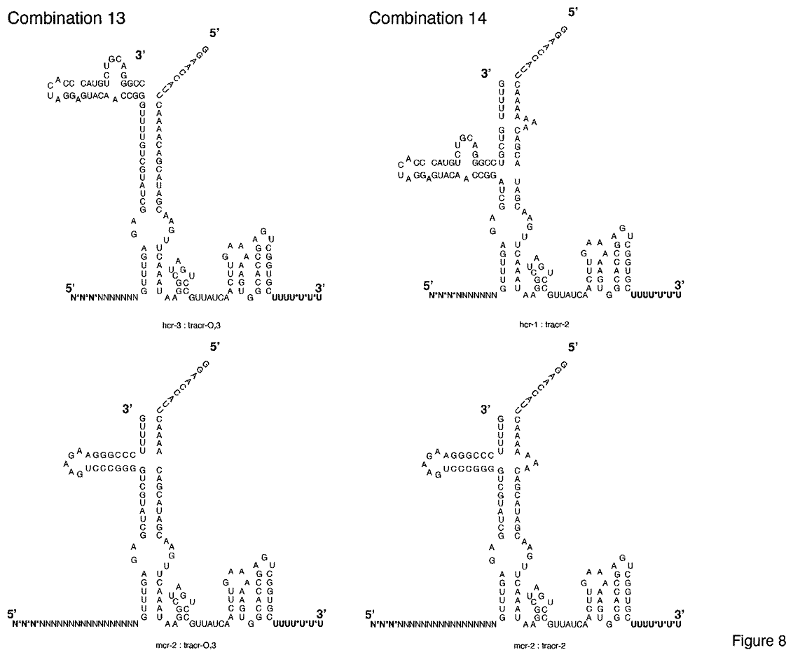

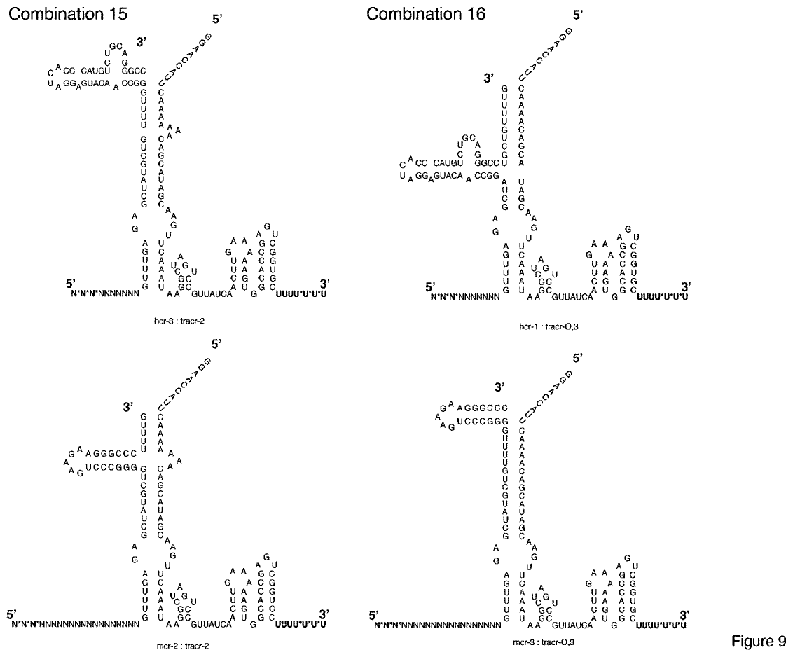

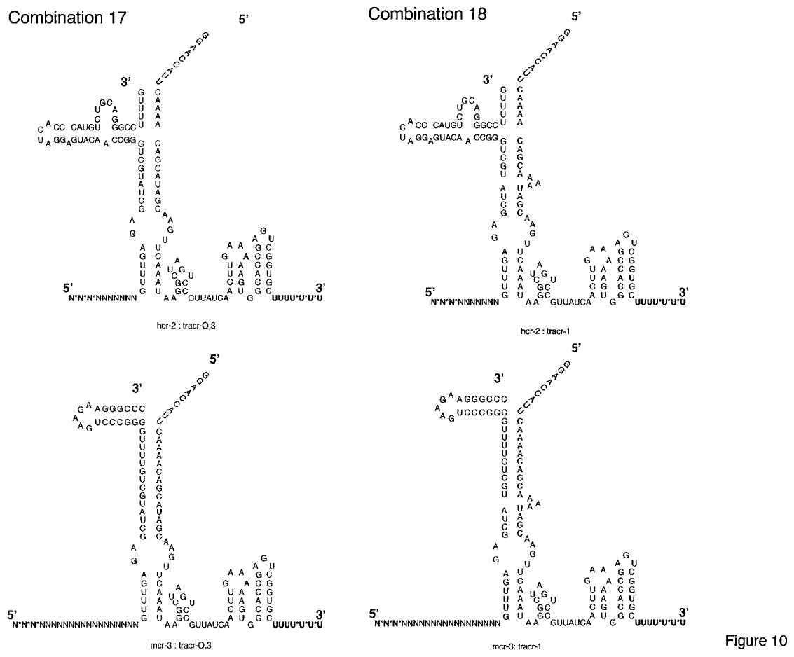

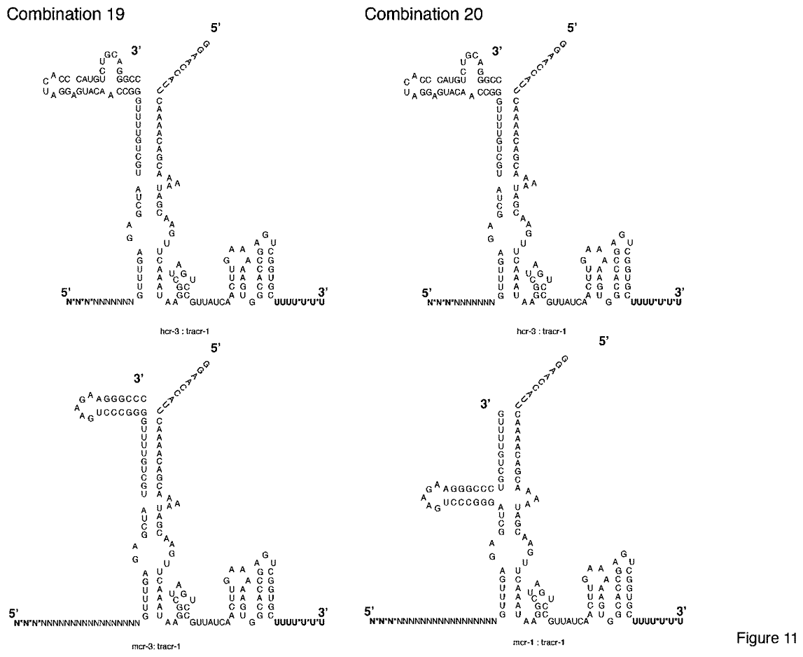

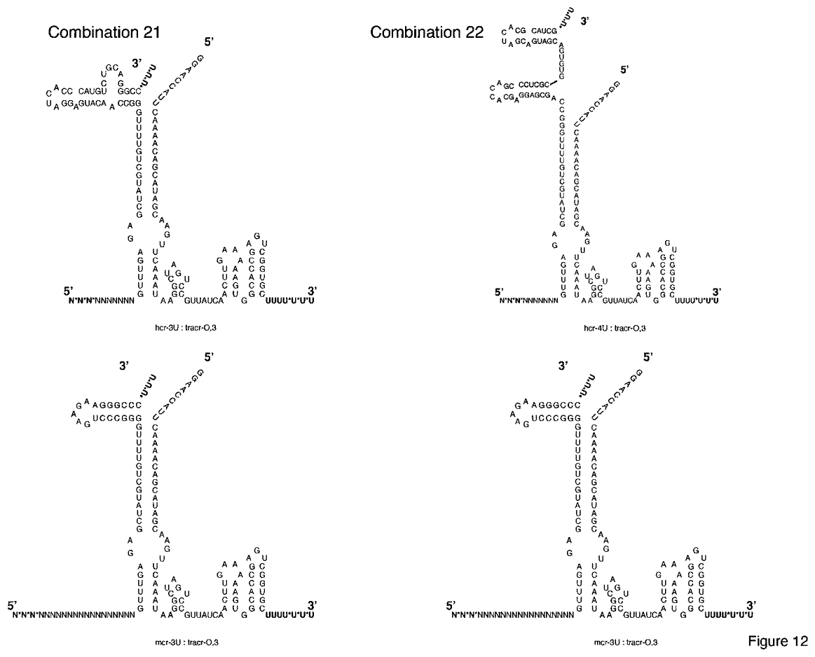

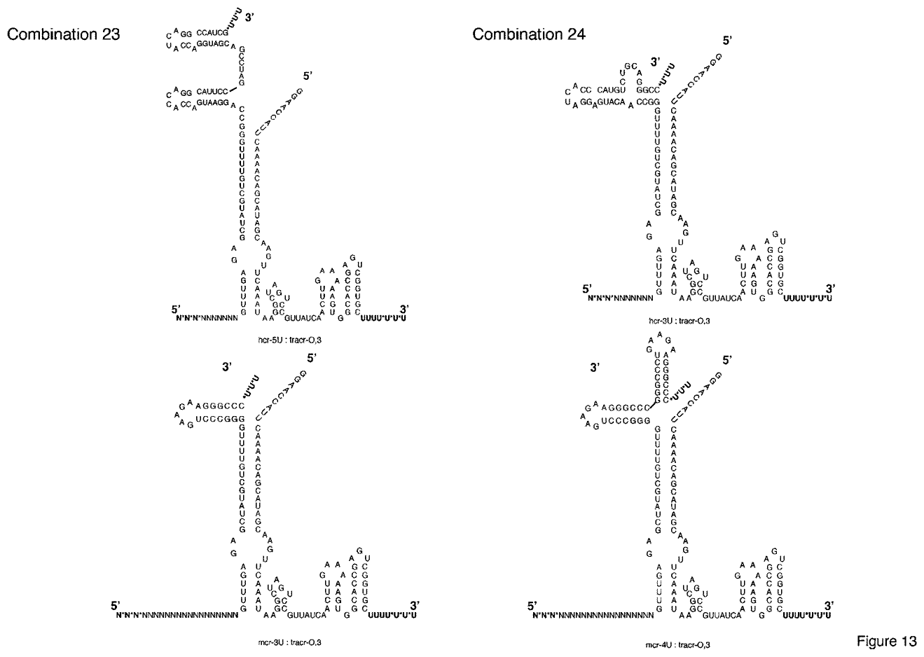

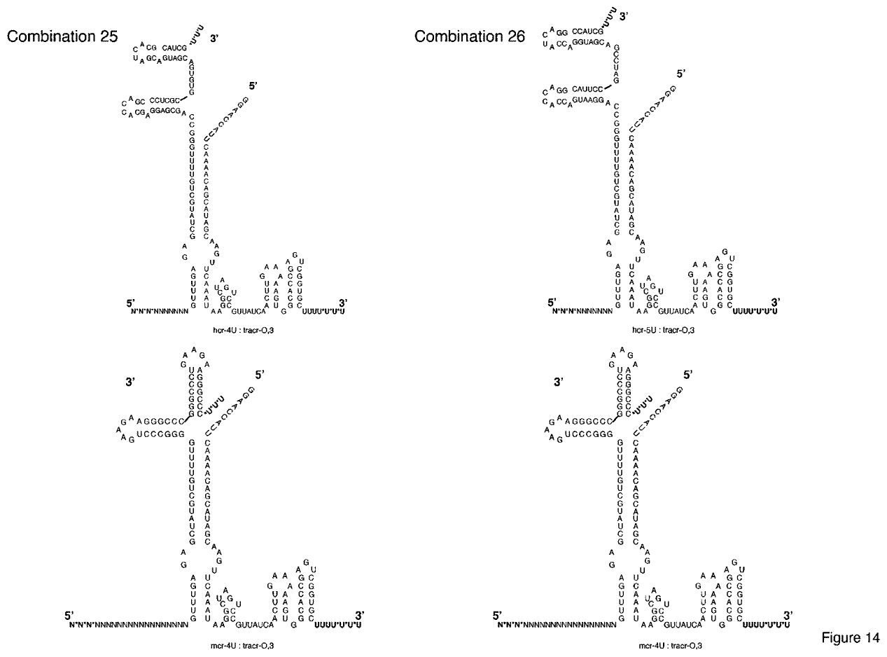

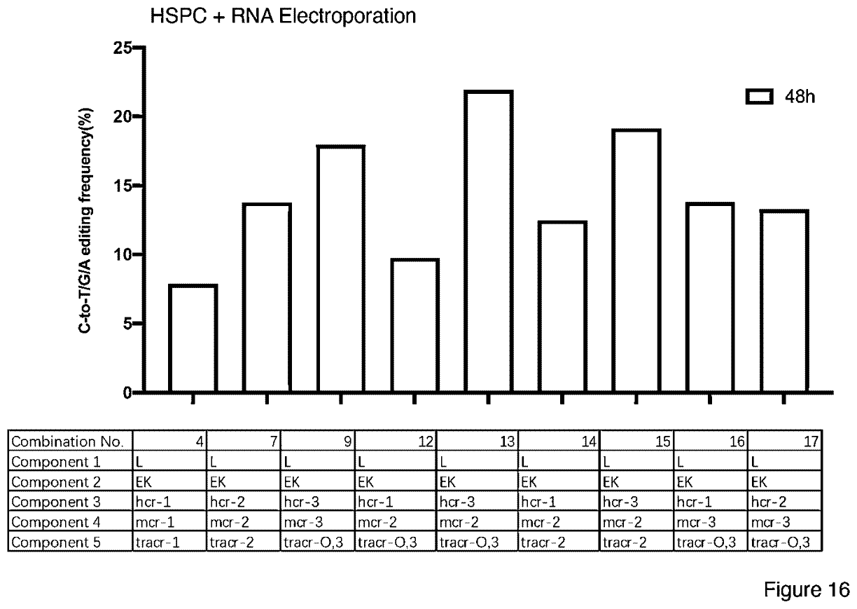

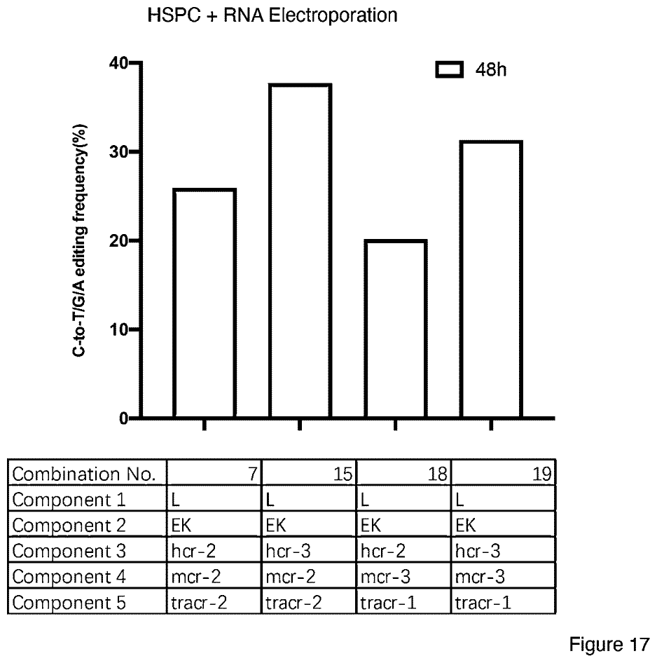

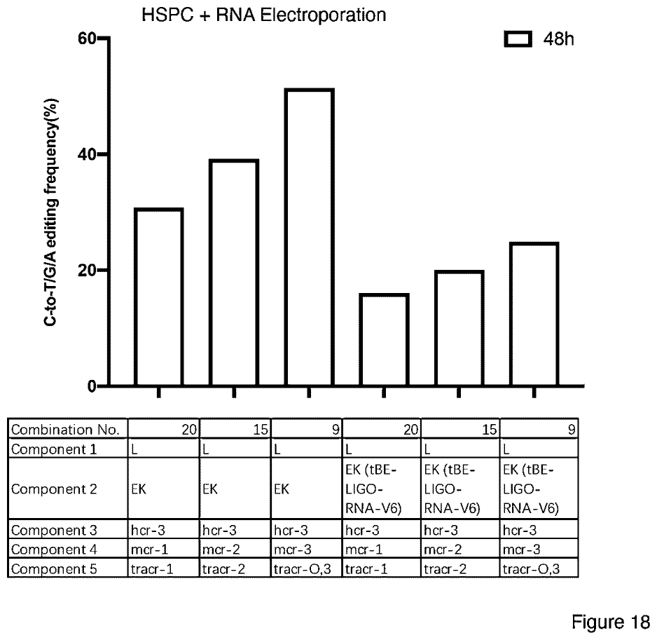

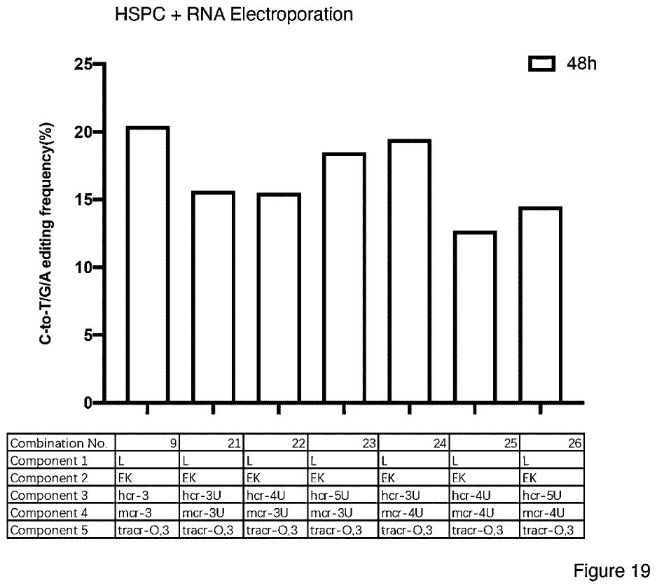

Original types of crRNA and tracrRNA are capable of guiding nCas9-mediated DNA location (FIG. 2, Combination 1). The crRNAs and the tracrRNAs in the current LigoRNA system are further modified. In some embodiments, an MS2 or boxB hairpin is fused to crRNA in multiple different sites. (FIGS. 2-14). In some embodiments, at least one nucleotide in the crRNAs and the tracrRNAs is modified, such as by a 2′-O-methyl modification and/or 3′-phosphorothioate modification. Multiple combinations of hcr-tracrRNA and mcr-tracrRNA base-paired structures are designed and tested. (FIGS. 2-19, Table 9-10).

LigoRNA system can be used in any CRISPR-base gene editing systems, such as a base editor system (BE) and a transformer base editor system (tBE). For example, a dual-RNA structure formed by a trans-activating crRNA (tracrRNA) and a ligand-bound CRISPR RNA (crRNA) locates and binds a target nucleotide sequence, such as DNA sequence and RNA sequence. Site-specific editing occurs at locations determined by both base-pairing complementarity between the crRNA and the target DNA, and the binding of Cas protein at protospacer adjacent motif (PAM).

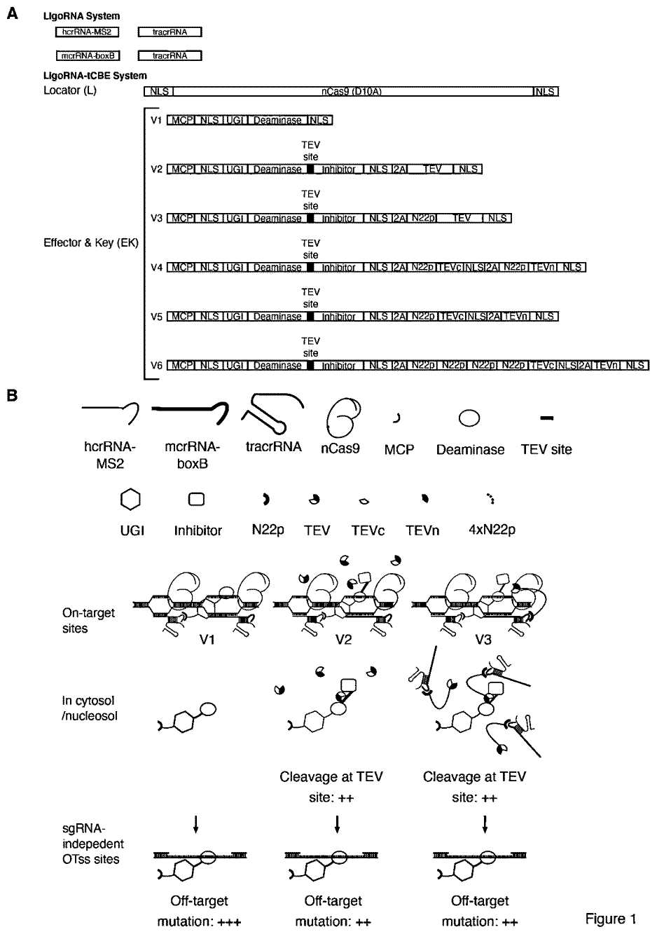

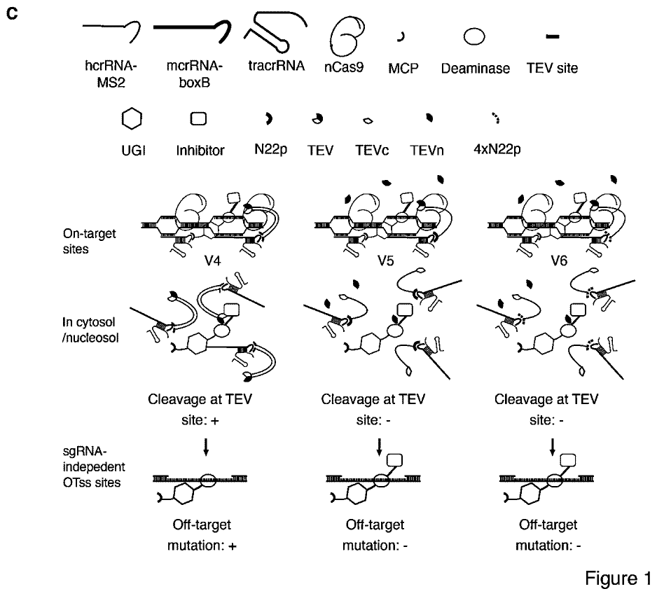

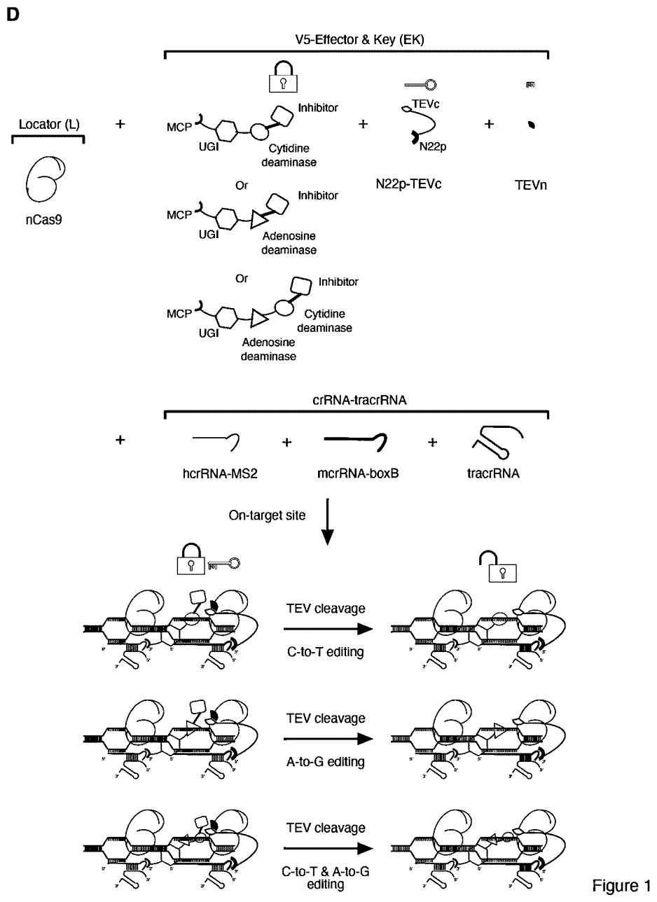

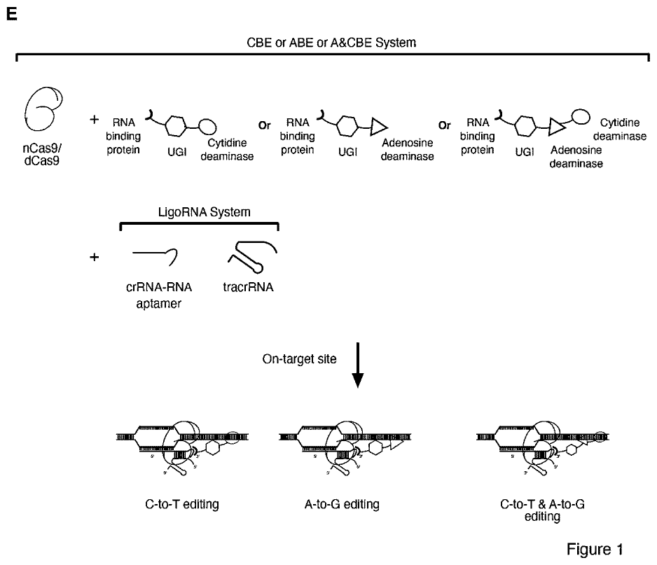

In some embodiments, the LigoRNA system is used in a base editor. FIG. 1(E) shows three exemplary embodiments of the LigoRNA-based gene editing system. It is understood that LigoRNA-based gene editing systems are not limited to what is shown in FIG. 1(E). For example, in some embodiments, the deaminase is directly linked or fused to the Cas protein.

In some embodiments, the LigoRNA system is used in a transformer base editor. FIGS. 1(A-D) provides exemplary embodiments of the LigoRNA-based tBE system. In some embodiments, the engineered crRNA is base-paired with a tracrRNA to form a dual-RNA structure that directs the nCas9 to locate the target DNA. For example, a LigoRNA-based tBE system comprises one main LigoRNA structure (mcr-tracrRNA, normally 20 nt base-paired to the target DNA) that binds at the target genomic site and one helper LigoRNA structure (hcr-tracrRNA, normally 10 to 20 nt base-paired to the target DNA) that binds at a nearby region (preferably upstream to the target genomic site). The binding of two LigoRNA structures can guide the components of tBE system to correctly assemble at the target genomic site for base editing.

In some embodiments, the LigoRNA-based tBE system comprises two LigoRNA structures: an mcrRNA-tracrRNA base-paired structure and an hcrRNA-tracrRNA base-paired structure. In some embodiments, the mcrRNA contains a boxB hairpin to generate an R-loop region for intended base editing and the hcrRNA contains an MS2 hairpin to recruit an APOBEC link to a cytosine deaminase inhibitor (dCDI) domain through a TEV protease cleavage site. To cleave off the dCDI domain at the on-target sites, the N22p-fused TEVc is recruited by the boxB-containing mcrRNA, working as the key in tBE system with free TEVn. In some embodiments, mcrRNA and hcrRNA form a base-paired structure with the same tracrRNA to locate at target DNA, and the dCDI domain is cleaved off at the target sites to induce efficient base editing.

Engineered crRNAs and tracrRNAs

In an aspect, the present disclosure provides an engineered CRISPR RNA (crRNA) comprising a spacer sequence and a linker sequence, wherein the linker sequence comprises at least one protein-binding motif, wherein the protein-binding motif is an RNA aptamer motif. In some embodiments, the protein binding motif is selected from MS2, PP7, boxB, SfMu hairpin motif, telomerase Ku, and Sm7 binding motif, or a variant thereof. Aptamers are single-stranded oligonucleotides that fold into defined architectures and selectively bind to a specific target, including proteins, peptides, carbohydrates, small molecules, toxins, and even live cells.

In some embodiments of the engineered crRNA described herein, the crRNA is any one of SEQ ID NOs: 288-301. It is noted that the string of “N” in SEQ ID NOs: 288-301 corresponds to a spacer sequence, which can be determined according to the desired target sequence. The number of “N”s in each of SEQ ID NOs: 288-301 can be shorter or longer than indicated in these sequences.

In some embodiments of the engineered crRNA described herein, the linker sequence is any one of SEQ ID Nos: 1-3 and 149-151.

In some embodiments of the engineered crRNA described herein, the linker sequence is any one of SEQ ID NOs: 4-7 and 152-153.

In some embodiments of the engineered crRNA described herein, the engineered crRNA is any one of SEQ ID NOs: 13-21, 154-158, 302-307, and 364-395.

In some embodiments of the engineered crRNA described herein, the crRNA is capable of forming a base-pair structure with a trans-activating crRNA (tracrRNA).

In some embodiments of the engineered crRNA described herein, the tracrRNA is any one of SEQ ID NOs: 10-12. In some embodiments, the tracrRNA is any one of SEQ ID NOs: 22-24.

In some embodiments of the engineered crRNA described herein, the engineered crRNA comprises at least one nucleotide with modification. In some embodiments, the modification is selected from 2′-O-alkyl, 2′-substituted alkoxy, 2′-substituted alkyl, 2′-halo, 3′-phosphorothioate, bridged nucleic acid (BNA), and locked nucleic acid (LNA). In some embodiments, the at least one nucleotide with modification is any one of the first three nucleotides from 3′-end of the engineered crRNA.

In an aspect, the present disclosure provides an engineered trans-activating crRNA (tracrRNA) of SEQ ID NO: 11 or SEQ ID NO: 12.

In an aspect, the present disclosure provides an engineered trans-activating crRNA (tracrRNA) of any one of SEQ ID NOs: 22-24.

In some embodiments of the engineered tracrRNA described herein, the engineered tracrRNA comprises at least one nucleotide with modification. In some embodiments, the modification is selected from 2′-O-alkyl, 2′-substituted alkoxy, 2′-substituted alkyl, 2′-halo, 3′-phosphorothioate, bridged nucleic acid (BNA), and locked nucleic acid (LNA). In some embodiments, the at least one nucleotide with modification is any one of the first three nucleotides from 3′-end of the engineered tracrRNA.

In some embodiments of the crRNA and/or tracrRNA described herein, the crRNA and/or tracrRNA comprises at least one nucleotide with modification. In some embodiments, the modification is selected from 2′-O-alkyl (such as 2′-O-methyl), 2′-substituted alkoxy, 2′-substituted alkyl, 2′-halo (such as 2′-fluoro), 3′-phosphorothioate, bridged nucleic acid (BNA), and locked nucleic acid (LNA). In some embodiments, the crRNA and/or tracrRNA comprises nucleotides comprising 2′-O-methyl and 3′-phosphorothioate. In some embodiments, the first three nucleotides from the 5′-end of the crRNA and/or tracrRNA are modified with 2′-O-methyl and 3′-phosphorothioate. In some embodiments, the first three nucleotides from the 3′-end of the crRNA and/or tracrRNA are modified with 2′-O-methyl, and the second to fourth nucleotides from the 3′-end of the crRNA and/or tracrRNA are modified with 3′-phosphorothioate. In some embodiments, the first three nucleotides from the 5′-end of the crRNA and/or tracrRNA are modified with 2′-O-methyl and 3′-phosphorothioate, and the first three nucleotides from the 3′-end of the crRNA and/or tracrRNA are modified with 2′-O-methyl, and the second to fourth nucleotides from the 3′-end of the crRNA and/or tracrRNA are modified with 3′-phosphorothioate.

In an aspect, the present disclosure provides a kit comprising an engineered crRNA described herein.

In an aspect, the present disclosure provides a kit comprising a first engineered crRNA described herein of any one of SEQ ID NOs: 1-3 and 149-151 and a second engineered crRNA described herein of any one of SEQ ID NOs: 4-7 and 152-153.

In some embodiments of the kit described herein, comprising a first engineered crRNA and a second engineered crRNA, wherein the first engineered crRNA comprises a first linker sequence and the second engineered crRNA comprises a second linker sequence, and wherein the first linker sequence and the second linker sequence are
a. SEQ ID NO: 1 and SEQ ID NO: 8, respectively; or
b. SEQ ID NO: 1 and SEQ ID NO: 4, respectively; or
c. SEQ ID NO: 2 and SEQ ID NO: 8, respectively; or
d. SEQ ID NO: 2 and SEQ ID NO: 5, respectively; or
e. SEQ ID NO: 3 and SEQ ID NO: 8, respectively; or
f. SEQ ID NO: 3 and SEQ ID NO: 6, respectively; or
g. SEQ ID NO: 1 and SEQ ID NO: 7, respectively; or
h. SEQ ID NO: 1 and SEQ ID NO: 5, respectively; or
i. SEQ ID NO: 3 and SEQ ID NO: 5, respectively; or
j. SEQ ID NO: 1 and SEQ ID NO: 6, respectively; or
k. SEQ ID NO: 2 and SEQ ID NO: 6, respectively; or
l. SEQ ID NO: 3 and SEQ ID NO: 6, respectively; or
m. SEQ ID NO: 3 and SEQ ID NO: 4, respectively; or
n. SEQ ID NO: 149 and SEQ ID NO: 152, respectively; or
o. SEQ ID NO: 150 and SEQ ID NO: 152, respectively; or
p. SEQ ID NO: 151 and SEQ ID NO: 152, respectively; or
q. SEQ ID NO: 149 and SEQ ID NO: 153, respectively; or
r. SEQ ID NO: 150 and SEQ ID NO: 153, respectively; or
s. SEQ ID NO: 151 and SEQ ID NO: 153, respectively.

In some embodiments of the kit described herein, the kit comprises a first engineered crRNA and a second engineered crRNA, wherein the first engineered crRNA and the second engineered crRNA are
a. SEQ ID NO: 21 and SEQ ID NO: 20, respectively; or
b. SEQ ID NO: 13 and SEQ ID NO: 20, respectively; or
c. SEQ ID NO: 13 and SEQ ID NO: 16, respectively; or
d. SEQ ID NO: 14 and SEQ ID NO: 20, respectively; or
e. SEQ ID NO: 14 and SEQ ID NO: 17, respectively; or
f. SEQ ID NO: 15 and SEQ ID NO: 20, respectively; or
g. SEQ ID NO: 15 and SEQ ID NO: 18, respectively; or
h. SEQ ID NO: 13 and SEQ ID NO: 19, respectively; or
i. SEQ ID NO: 13 and SEQ ID NO: 17, respectively; or
j. SEQ ID NO: 15 and SEQ ID NO: 17, respectively; or
k. SEQ ID NO: 13 and SEQ ID NO: 18, respectively; or
l. SEQ ID NO: 14 and SEQ ID NO: 18, respectively; or
m. SEQ ID NO: 15 and SEQ ID NO: 18, respectively; or
n. SEQ ID NO: 15 and SEQ ID NO: 16, respectively; or
o. SEQ ID NO: 154 and SEQ ID NO: 157, respectively; or
p. SEQ ID NO: 155 and SEQ ID NO: 157, respectively; or
q. SEQ ID NO: 156 and SEQ ID NO: 157, respectively; or
r. SEQ ID NO: 154 and SEQ ID NO: 158, respectively; or
s. SEQ ID NO: 155 and SEQ ID NO: 158, respectively; or
qq. SEQ ID NO: 156 and SEQ ID NO: 158, respectively; or
rr. SEQ ID NO: 302 and SEQ ID NO: 303, respectively; or
ss. SEQ ID NO: 304 and SEQ ID NO: 305, respectively; or
t. SEQ ID NO: 306 and SEQ ID NO: 307, respectively; or
u. SEQ ID NO: 364 and SEQ ID NO: 365, respectively; or
v. SEQ ID NO: 366 and SEQ ID NO: 367, respectively; or
w. SEQ ID NO: 368 and SEQ ID NO: 369, respectively; or
x. SEQ ID NO: 390, and SEQ ID NO: 389, respectively; or
y. SEQ ID NO: 382, and SEQ ID NO: 389, respectively; or
z. SEQ ID NO: 382, and SEQ ID NO: 385, respectively; or
aa. SEQ ID NO: 382, and SEQ ID NO: 385, respectively; or
bb. SEQ ID NO: 383, and SEQ ID NO: 389, respectively; or
cc. SEQ ID NO: 383, and SEQ ID NO: 386, respectively; or
dd. SEQ ID NO: 383, and SEQ ID NO: 386, respectively; or
ee. SEQ ID NO: 384, and SEQ ID NO: 389, respectively; or
ff. SEQ ID NO: 384, and SEQ ID NO: 387, respectively; or
gg. SEQ ID NO: 382, and SEQ ID NO: 388, respectively; or
hh. SEQ ID NO: 382, and SEQ ID NO: 388, respectively; or
ii. SEQ ID NO: 382, and SEQ ID NO: 386, respectively; or
jj. SEQ ID NO: 384, and SEQ ID NO: 386, respectively; or
kk. SEQ ID NO: 382, and SEQ ID NO: 386, respectively; or
ll. SEQ ID NO: 384, and SEQ ID NO: 386, respectively; or
mm. SEQ ID NO: 382, and SEQ ID NO: 387, respectively; or
nn. SEQ ID NO: 383, and SEQ ID NO: 387, respectively; or
oo. SEQ ID NO: 383, and SEQ ID NO: 387, respectively; or
pp. SEQ ID NO: 384, and SEQ ID NO: 387, respectively; or
qq. SEQ ID NO: 384, and SEQ ID NO: 385, respectively; or
rr. SEQ ID NO: 370 and SEQ ID NO: 371, respectively; or
ss. SEQ ID NO: 372 and SEQ ID NO: 373, respectively; or
tt. SEQ ID NO: 374 and SEQ ID NO: 375, respectively or
uu. SEQ ID NO: 376 and SEQ ID NO: 377, respectively; or
vv. SEQ ID NO: 378 and SEQ ID NO: 379, respectively; or
ww. SEQ ID NO: 380 and SEQ ID NO: 381, respectively.

In some embodiments of the kit described herein, it further comprises at least one tracrRNA, wherein the at least one tracrRNA are the same or different.

In some embodiments of the kit described herein, each of the at least one tracrRNA is selected from SEQ ID Nos: 10-12. In some embodiments, the each tracrRNA is any one of SEQ ID Nos: 22-24.

In some embodiments of the kit described herein, wherein the first engineered crRNA comprises a first linker sequence and the second engineered crRNA comprises a second linker sequence, and wherein the first linker sequence, the second linker sequence, and the tracrRNA are
a. SEQ ID NO: 1, SEQ ID NO: 8, and SEQ ID NO: 10, respectively; or
b. SEQ ID NO: 1, SEQ ID NO: 4, and SEQ ID NO: 10, respectively; or
c. SEQ ID NO: 1, SEQ ID NO: 4, and SEQ ID NO: 11, respectively; or
d. SEQ ID NO: 2, SEQ ID NO: 8, and SEQ ID NO: 10, respectively; or
e. SEQ ID NO: 2, SEQ ID NO: 5, and SEQ ID NO: 10, respectively; or
f. SEQ ID NO: 2, SEQ ID NO: 5, and SEQ ID NO: 12, respectively; or
g. SEQ ID NO: 3, SEQ ID NO: 8, and SEQ ID NO: 10, respectively; or
h. SEQ ID NO: 3, SEQ ID NO: 6, and SEQ ID NO: 10, respectively; or
i. SEQ ID NO: 1, SEQ ID NO: 7, and SEQ ID NO: 10, respectively; or
j. SEQ ID NO: 1, SEQ ID NO: 7, and SEQ ID NO: 11, respectively; or
k. SEQ ID NO: 1, SEQ ID NO: 5, and SEQ ID NO: 10, respectively; or
l. SEQ ID NO: 3, SEQ ID NO: 5, and SEQ ID NO: 10, respectively; or
m. SEQ ID NO: 1, SEQ ID NO: 5, and SEQ ID NO: 12, respectively; or
n. SEQ ID NO: 3, SEQ ID NO: 5, and SEQ ID NO: 12, respectively; or
o. SEQ ID NO: 1, SEQ ID NO: 6, and SEQ ID NO: 10, respectively; or
p. SEQ ID NO: 2, SEQ ID NO: 6, and SEQ ID NO: 10, respectively; or
q. SEQ ID NO: 2, SEQ ID NO: 6, and SEQ ID NO: 11, respectively; or
r. SEQ ID NO: 3, SEQ ID NO: 6, and SEQ ID NO: 11, respectively; or
s. SEQ ID NO: 3, SEQ ID NO: 4, and SEQ ID NO: 11, respectively; or
t. SEQ ID NO: 149, SEQ ID NO: 152, and SEQ ID NO: 10, respectively; or
u. SEQ ID NO: 150, SEQ ID NO: 152, and SEQ ID NO: 10, respectively; or
v. SEQ ID NO: 151, SEQ ID NO: 152, and SEQ ID NO: 10, respectively; or
w. SEQ ID NO: 149, SEQ ID NO: 153, and SEQ ID NO: 10, respectively; or
x. SEQ ID NO: 150, SEQ ID NO: 153, and SEQ ID NO: 10, respectively; or
y. SEQ ID NO: 151, SEQ ID NO: 153, and SEQ ID NO: 10, respectively.

In some embodiments of the kit described herein, the first engineered crRNA, the second engineered crRNA, and the tracrRNA are
a. SEQ ID NO: 21, SEQ ID NO: 20, and SEQ ID NO: 22, respectively; or
b. SEQ ID NO: 13, SEQ ID NO: 20, and SEQ ID NO: 22, respectively; or
c. SEQ ID NO: 13, SEQ ID NO: 16, and SEQ ID NO: 22, respectively; or
d. SEQ ID NO: 13, SEQ ID NO: 16, and SEQ ID NO: 23, respectively; or
e. SEQ ID NO: 14, SEQ ID NO: 20, and SEQ ID NO: 22, respectively; or
f. SEQ ID NO: 14, SEQ ID NO: 17, and SEQ ID NO: 22, respectively; or
g. SEQ ID NO: 14, SEQ ID NO: 17, and SEQ ID NO: 24, respectively; or
h. SEQ ID NO: 15, SEQ ID NO: 20, and SEQ ID NO: 22, respectively; or
i. SEQ ID NO: 15, SEQ ID NO: 18, and SEQ ID NO: 22, respectively; or
j. SEQ ID NO: 13, SEQ ID NO: 19, and SEQ ID NO: 22, respectively; or
k. SEQ ID NO: 13, SEQ ID NO: 19, and SEQ ID NO: 23, respectively; or
l. SEQ ID NO: 13, SEQ ID NO: 17, and SEQ ID NO: 22, respectively; or
m. SEQ ID NO: 15, SEQ ID NO: 17, and SEQ ID NO: 22, respectively; or
n. SEQ ID NO: 13, SEQ ID NO: 17, and SEQ ID NO: 24, respectively; or
o. SEQ ID NO: 15, SEQ ID NO: 17, and SEQ ID NO: 24, respectively; or
p. SEQ ID NO: 13, SEQ ID NO: 18, and SEQ ID NO: 22, respectively; or
q. SEQ ID NO: 14, SEQ ID NO: 18, and SEQ ID NO: 22, respectively; or
r. SEQ ID NO: 14, SEQ ID NO: 18, and SEQ ID NO: 23, respectively; or
s. SEQ ID NO: 15, SEQ ID NO: 18, and SEQ ID NO: 23, respectively; or
t. SEQ ID NO: 15, SEQ ID NO: 16, and SEQ ID NO: 23, respectively; or
u. SEQ ID NO: 154, SEQ ID NO: 157, and SEQ ID NO: 22, respectively; or
v. SEQ ID NO: 155, SEQ ID NO: 157, and SEQ ID NO: 22, respectively; or
w. SEQ ID NO: 156, SEQ ID NO: 157, and SEQ ID NO: 22, respectively; or
x. SEQ ID NO: 154, SEQ ID NO: 158, and SEQ ID NO: 22, respectively; or
y. SEQ ID NO: 155, SEQ ID NO: 158, and SEQ ID NO: 22, respectively; or
tt. SEQ ID NO: 156, SEQ ID NO: 158, and SEQ ID NO: 22, respectively; or
uu. SEQ ID NO: 302, SEQ ID NO: 303, and SEQ ID NO: 22, respectively; or
vv. SEQ ID NO: 304, SEQ ID NO: 305, and SEQ ID NO: 22, respectively; or
z. SEQ ID NO: 306, SEQ ID NO: 307, and SEQ ID NO: 22, respectively; or
aa. SEQ ID NO: 364, SEQ ID NO: 365, and SEQ ID NO: 22, respectively; or
bb. SEQ ID NO: 366, SEQ ID NO: 367, and SEQ ID NO: 22, respectively; or
cc. SEQ ID NO: 368, SEQ ID NO: 369, and SEQ ID NO: 22, respectively; or
dd. SEQ ID NO: 390, SEQ ID NO: 389, and SEQ ID NO: 10, respectively; or
ee. SEQ ID NO: 382, SEQ ID NO: 389, and SEQ ID NO: 10, respectively; or
ff. SEQ ID NO: 382, SEQ ID NO: 385, and SEQ ID NO: 10, respectively; or
gg. SEQ ID NO: 382, SEQ ID NO: 385, and SEQ ID NO: 11, respectively; or
hh. SEQ ID NO: 383, SEQ ID NO: 389, and SEQ ID NO: 10, respectively; or
ii. SEQ ID NO: 383, SEQ ID NO: 386, and SEQ ID NO: 10, respectively; or
jj. SEQ ID NO: 383, SEQ ID NO: 386, and SEQ ID NO: 12, respectively; or
kk. SEQ ID NO: 384, SEQ ID NO: 389, and SEQ ID NO: 10, respectively; or
ll. SEQ ID NO: 384, SEQ ID NO: 387, and SEQ ID NO: 10, respectively; or
mm. SEQ ID NO: 382, SEQ ID NO: 388, and SEQ ID NO: 10, respectively; or
nn. SEQ ID NO: 382, SEQ ID NO: 388, and SEQ ID NO: 11, respectively; or
oo. SEQ ID NO: 382, SEQ ID NO: 386, and SEQ ID NO: 10, respectively; or
pp. SEQ ID NO: 384, SEQ ID NO: 386, and SEQ ID NO: 10, respectively; or
qq. SEQ ID NO: 382, SEQ ID NO: 386, and SEQ ID NO: 12, respectively; or
rr. SEQ ID NO: 384, SEQ ID NO: 386, and SEQ ID NO: 12, respectively; or
ss. SEQ ID NO: 382, SEQ ID NO: 387, and SEQ ID NO: 10, respectively; or
tt. SEQ ID NO: 383, SEQ ID NO: 387, and SEQ ID NO: 10, respectively; or
uu. SEQ ID NO: 383, SEQ ID NO: 387, and SEQ ID NO: 11, respectively; or
vv. SEQ ID NO: 384, SEQ ID NO: 387, and SEQ ID NO: 11, respectively; or
ww. SEQ ID NO: 384, SEQ ID NO: 385, and SEQ ID NO: 11, respectively; or
xx. SEQ ID NO: 370, SEQ ID NO: 371, and SEQ ID NO: 10, respectively; or
yy. SEQ ID NO: 372, SEQ ID NO: 373, and SEQ ID NO: 10, respectively; or
zz. SEQ ID NO: 374, SEQ ID NO: 375, and SEQ ID NO: 10, respectively or
aaa. SEQ ID NO: 376, SEQ ID NO: 377, and SEQ ID NO: 10, respectively; or
bbb. SEQ ID NO: 378, SEQ ID NO: 379, and SEQ ID NO: 10, respectively; or
ccc. SEQ ID NO: 380, SEQ ID NO: 381, and SEQ ID NO: 10, respectively.

In some embodiments of the kit described herein, it further comprises at least one CRISPR associated protein (Cas protein) or a variant thereof, or at least one polynucleotide encoding the at least one Cas protein or a variant thereof, wherein the at least one Cas protein or a variant thereof are the same or different.

### LigoRNA-Based Gene Editing Systems

In an aspect, the present disclosure provides a gene editing system comprising a helper crRNA (hcrRNA) and a main crRNA (mcrRNA), or at least one DNA polynucleotide encoding the hcrRNA and/or the mcrRNA, wherein the hcrRNA comprises a first spacer sequence and a first linker sequence, wherein the first linker sequence comprises a first protein-binding motif, and the mcrRNA comprises a second spacer sequence and a second linker sequence, wherein the second linker sequence optionally comprises a second protein binding motif.

In some embodiments of the gene editing system described herein, the hcrRNA is the engineered crRNA described herein of any one of SEQ ID NOs: 1-3 and 149-151.

In some embodiments of the gene editing system described herein, the mcrRNA is the engineered crRNA described herein of any one of SEQ ID NOs: 4-7 and 152-153.

In some embodiments of the gene editing system described herein, the hcrRNA is the engineered crRNA described herein of any one of SEQ ID NOs: 1-3 and 149-151, and the mcrRNA is the engineered crRNA described herein of any one of SEQ ID NOs: 4-7 and 152-153.

In some embodiments of the gene editing system described herein, the first linker sequence and the second linker sequence are
a. SEQ ID NO: 1 and SEQ ID NO: 8, respectively; or
b. SEQ ID NO: 1 and SEQ ID NO: 4, respectively; or
c. SEQ ID NO: 2 and SEQ ID NO: 8, respectively; or
d. SEQ ID NO: 2 and SEQ ID NO: 5, respectively; or
e. SEQ ID NO: 3 and SEQ ID NO: 8, respectively; or
f. SEQ ID NO: 3 and SEQ ID NO: 6, respectively; or
g. SEQ ID NO: 1 and SEQ ID NO: 7, respectively; or
h. SEQ ID NO: 1 and SEQ ID NO: 5, respectively; or
i. SEQ ID NO: 3 and SEQ ID NO: 5, respectively; or
j. SEQ ID NO: 1 and SEQ ID NO: 6, respectively; or
k. SEQ ID NO: 2 and SEQ ID NO: 6, respectively; or
l. SEQ ID NO: 3 and SEQ ID NO: 6, respectively; or
m. SEQ ID NO: 3 and SEQ ID NO: 4, respectively; or
n. SEQ ID NO: 149 and SEQ ID NO: 152, respectively; or
o. SEQ ID NO: 150 and SEQ ID NO: 152, respectively; or
p. SEQ ID NO: 151 and SEQ ID NO: 152, respectively; or
q. SEQ ID NO: 149 and SEQ ID NO: 153, respectively; or
r. SEQ ID NO: 150 and SEQ ID NO: 153, respectively; or
s. SEQ ID NO: 151 and SEQ ID NO: 153, respectively.
t.

In some embodiments of the gene editing system described herein, the hcrRNA and the mcrRNA are
a. SEQ ID NO: 21 and SEQ ID NO: 20, respectively; or
b. SEQ ID NO: 13 and SEQ ID NO: 20, respectively; or
c. SEQ ID NO: 13 and SEQ ID NO: 16, respectively; or
d. SEQ ID NO: 14 and SEQ ID NO: 20, respectively; or
e. SEQ ID NO: 14 and SEQ ID NO: 17, respectively; or
f. SEQ ID NO: 15 and SEQ ID NO: 20, respectively; or
g. SEQ ID NO: 15 and SEQ ID NO: 18, respectively; or
h. SEQ ID NO: 13 and SEQ ID NO: 19, respectively; or
i. SEQ ID NO: 13 and SEQ ID NO: 17, respectively; or
j. SEQ ID NO: 15 and SEQ ID NO: 17, respectively; or
k. SEQ ID NO: 13 and SEQ ID NO: 18, respectively; or
l. SEQ ID NO: 14 and SEQ ID NO: 18, respectively; or
m. SEQ ID NO: 15 and SEQ ID NO: 18, respectively; or
n. SEQ ID NO: 15 and SEQ ID NO: 16, respectively; or
o. SEQ ID NO: 154 and SEQ ID NO: 157, respectively; or
p. SEQ ID NO: 155 and SEQ ID NO: 157, respectively; or
q. SEQ ID NO: 156 and SEQ ID NO: 157, respectively; or
r. SEQ ID NO: 154 and SEQ ID NO: 158, respectively; or
s. SEQ ID NO: 155 and SEQ ID NO: 158, respectively; or
ww. SEQ ID NO: 156 and SEQ ID NO: 158, respectively; or
xx. SEQ ID NO: 302 and SEQ ID NO: 303, respectively; or
yy. SEQ ID NO: 304 and SEQ ID NO: 305, respectively; or
t. SEQ ID NO: 306 and SEQ ID NO: 307, respectively; or
u. SEQ ID NO: 364 and SEQ ID NO: 365, respectively; or
v. SEQ ID NO: 366 and SEQ ID NO: 367, respectively; or
w. SEQ ID NO: 368 and SEQ ID NO: 369, respectively; or
x. SEQ ID NO: 390, and SEQ ID NO: 389, respectively; or
y. SEQ ID NO: 382, and SEQ ID NO: 389, respectively; or
z. SEQ ID NO: 382, and SEQ ID NO: 385, respectively; or
aa. SEQ ID NO: 382, and SEQ ID NO: 385, respectively; or
bb. SEQ ID NO: 383, and SEQ ID NO: 389, respectively; or
cc. SEQ ID NO: 383, and SEQ ID NO: 386, respectively; or
dd. SEQ ID NO: 383, and SEQ ID NO: 386, respectively; or
ee. SEQ ID NO: 384, and SEQ ID NO: 389, respectively; or
ff. SEQ ID NO: 384, and SEQ ID NO: 387, respectively; or
gg. SEQ ID NO: 382, and SEQ ID NO: 388, respectively; or
hh. SEQ ID NO: 382, and SEQ ID NO: 388, respectively; or
ii. SEQ ID NO: 382, and SEQ ID NO: 386, respectively; or
jj. SEQ ID NO: 384, and SEQ ID NO: 386, respectively; or
kk. SEQ ID NO: 382, and SEQ ID NO: 386, respectively; or
ll. SEQ ID NO: 384, and SEQ ID NO: 386, respectively; or
mm. SEQ ID NO: 382, and SEQ ID NO: 387, respectively; or
nn. SEQ ID NO: 383, and SEQ ID NO: 387, respectively; or
oo. SEQ ID NO: 383, and SEQ ID NO: 387, respectively; or
pp. SEQ ID NO: 384, and SEQ ID NO: 387, respectively; or
qq. SEQ ID NO: 384, and SEQ ID NO: 385, respectively; or
rr. SEQ ID NO: 370 and SEQ ID NO: 371, respectively; or
ss. SEQ ID NO: 372 and SEQ ID NO: 373, respectively; or
tt. SEQ ID NO: 374 and SEQ ID NO: 375, respectively or
uu. SEQ ID NO: 376 and SEQ ID NO: 377, respectively; or
vv. SEQ ID NO: 378 and SEQ ID NO: 379, respectively; or
ww. SEQ ID NO: 380 and SEQ ID NO: 381, respectively.

In some embodiments of the gene editing system described herein, it further comprises a first tracrRNA and a second tracrRNA, wherein the first tracrRNA and second tracrRNA are the same or different.

In some embodiments of the gene editing system described herein, the first tracrRNA and the second tracrRNA each has a sequence of any one of SEQ ID NO: 10-12. In some embodiments, the first and second tracrRNA is each selected from SEQ ID NOs: 22-24.

In some embodiments of the gene editing system described herein, the first linker sequence, the second linker sequence, the first tracrRNA, and the second tracrRNA are
a. SEQ ID NO: 1, SEQ ID NO: 8, SEQ ID NO: 10, and SEQ ID NO: 10, respectively; or
b. SEQ ID NO: 1, SEQ ID NO: 4, SEQ ID NO: 10, and SEQ ID NO: 10, respectively; or
c. SEQ ID NO: 1, SEQ ID NO: 4, SEQ ID NO: 11, and SEQ ID NO: 11, respectively; or
d. SEQ ID NO: 2, SEQ ID NO: 8, SEQ ID NO: 10, and SEQ ID NO: 10, respectively; or
e. SEQ ID NO: 2, SEQ ID NO: 5, SEQ ID NO: 10, and SEQ ID NO: 10, respectively; or
f. SEQ ID NO: 2, SEQ ID NO: 5, SEQ ID NO: 12, and SEQ ID NO: 12, respectively; or
g. SEQ ID NO: 3, SEQ ID NO: 8, SEQ ID NO: 10, and SEQ ID NO: 10, respectively; or
h. SEQ ID NO: 3, SEQ ID NO: 6, SEQ ID NO: 10, and SEQ ID NO: 10, respectively; or
i. SEQ ID NO: 1, SEQ ID NO: 7, SEQ ID NO: 10, and SEQ ID NO: 10, respectively; or
j. SEQ ID NO: 1, SEQ ID NO: 7, SEQ ID NO: 11, and SEQ ID NO: 11, respectively; or
k. SEQ ID NO: 1, SEQ ID NO: 5, SEQ ID NO: 10, and SEQ ID NO: 10, respectively; or
l. SEQ ID NO: 3, SEQ ID NO: 5, SEQ ID NO: 10, and SEQ ID NO: 10, respectively; or
m. SEQ ID NO: 1, SEQ ID NO: 5, SEQ ID NO: 12, and SEQ ID NO: 12, respectively; or
n. SEQ ID NO: 3, SEQ ID NO: 5, SEQ ID NO: 12, and SEQ ID NO: 12, respectively; or
o. SEQ ID NO: 1, SEQ ID NO: 6, SEQ ID NO: 10, and SEQ ID NO: 10, respectively; or
p. SEQ ID NO: 2, SEQ ID NO: 6, SEQ ID NO: 10, and SEQ ID NO: 10, respectively; or
q. SEQ ID NO: 2, SEQ ID NO: 6, SEQ ID NO: 11, and SEQ ID NO: 11, respectively; or
r. SEQ ID NO: 3, SEQ ID NO: 6, SEQ ID NO: 11, and SEQ ID NO: 11, respectively; or
s. SEQ ID NO: 3, SEQ ID NO: 4, SEQ ID NO: 11, and SEQ ID NO: 11, respectively; or
t. SEQ ID NO: 149, SEQ ID NO: 152, SEQ ID NO: 10, and SEQ ID NO: 10, respectively; or
u. SEQ ID NO: 150, SEQ ID NO: 152, SEQ ID NO: 10, and SEQ ID NO: 10, respectively; or
v. SEQ ID NO: 151, SEQ ID NO: 152, SEQ ID NO: 10, and SEQ ID NO: 10, respectively; or
w. SEQ ID NO: 149, SEQ ID NO: 153, SEQ ID NO: 10, and SEQ ID NO: 10, respectively; or
x. SEQ ID NO: 150, SEQ ID NO: 153, SEQ ID NO: 10, and SEQ ID NO: 10, respectively; or
y. SEQ ID NO: 151, SEQ ID NO: 153, SEQ ID NO: 10, and SEQ ID NO: 10, respectively.

In some embodiments of the gene editing system described herein, the hcrRNA, the mcrRNA, the first tracrRNA, and the second tracrRNA are
a. SEQ ID NO: 21, SEQ ID NO: 20, SEQ ID NO: 22, and SEQ ID NO: 22, respectively; or
b. SEQ ID NO: 13, SEQ ID NO: 20, SEQ ID NO: 22, and SEQ ID NO: 22, respectively; or
c. SEQ ID NO: 13, SEQ ID NO: 16, SEQ ID NO: 22, and SEQ ID NO: 22, respectively; or
d. SEQ ID NO: 13, SEQ ID NO: 16, SEQ ID NO: 23, and SEQ ID NO: 23, respectively; or
e. SEQ ID NO: 14, SEQ ID NO: 20, SEQ ID NO: 22, and SEQ ID NO: 22, respectively; or
f. SEQ ID NO: 14, SEQ ID NO: 17, SEQ ID NO: 22, and SEQ ID NO: 22, respectively; or
g. SEQ ID NO: 14, SEQ ID NO: 17, SEQ ID NO: 24, and SEQ ID NO: 24, respectively; or
h. SEQ ID NO: 15, SEQ ID NO: 20, SEQ ID NO: 22, and SEQ ID NO: 22, respectively; or
i. SEQ ID NO: 15, SEQ ID NO: 18, SEQ ID NO: 22, and SEQ ID NO: 22, respectively; or
j. SEQ ID NO: 13, SEQ ID NO: 19, SEQ ID NO: 22, and SEQ ID NO: 22, respectively; or
k. SEQ ID NO: 13, SEQ ID NO: 19, SEQ ID NO: 23, and SEQ ID NO: 23, respectively; or
l. SEQ ID NO: 13, SEQ ID NO: 17, SEQ ID NO: 22, and SEQ ID NO: 22, respectively; or
m. SEQ ID NO: 15, SEQ ID NO: 17, SEQ ID NO: 22, and SEQ ID NO: 22, respectively; or
n. SEQ ID NO: 13, SEQ ID NO: 17, SEQ ID NO: 24, and SEQ ID NO: 24, respectively; or
o. SEQ ID NO: 15, SEQ ID NO: 17, SEQ ID NO: 24, and SEQ ID NO: 24, respectively; or
p. SEQ ID NO: 13, SEQ ID NO: 18, SEQ ID NO: 22, and SEQ ID NO: 22, respectively; or
q. SEQ ID NO: 14, SEQ ID NO: 18, SEQ ID NO: 22, and SEQ ID NO: 22, respectively; or
r. SEQ ID NO: 14, SEQ ID NO: 18, SEQ ID NO: 23, and SEQ ID NO: 23, respectively; or
s. SEQ ID NO: 15, SEQ ID NO: 18, SEQ ID NO: 23, and SEQ ID NO: 23, respectively; or
t. SEQ ID NO: 15, SEQ ID NO: 16, SEQ ID NO: 23, and SEQ ID NO: 23, respectively; or
u. SEQ ID NO: 154, SEQ ID NO: 157, SEQ ID NO: 22, and SEQ ID NO: 22, respectively; or
v. SEQ ID NO: 155, SEQ ID NO: 157, SEQ ID NO: 22, and SEQ ID NO: 22, respectively; or
w. SEQ ID NO: 156, SEQ ID NO: 157, SEQ ID NO: 22, and SEQ ID NO: 22, respectively; or
x. SEQ ID NO: 154, SEQ ID NO: 158, SEQ ID NO: 22, and SEQ ID NO: 22, respectively; or
y. SEQ ID NO: 155, SEQ ID NO: 158, SEQ ID NO: 22, and SEQ ID NO: 22, respectively; or
zz. SEQ ID NO: 156, SEQ ID NO: 158, SEQ ID NO: 22, and SEQ ID NO: 22, respectively; or
aaa. SEQ ID NO: 302, SEQ ID NO: 303, SEQ ID NO: 22 and SEQ ID NO: 22, respectively; or
bbb. SEQ ID NO: 304, SEQ ID NO: 305, SEQ ID NO: 22 and SEQ ID NO: 22, respectively; or
z. SEQ ID NO: 306, SEQ ID NO: 307, SEQ ID NO: 22 and SEQ ID NO: 22, respectively; or
aa. SEQ ID NO: 364, SEQ ID NO: 365, SEQ ID NO: 22 and SEQ ID NO: 22, respectively; or
bb. SEQ ID NO: 366, SEQ ID NO: 367, SEQ ID NO: 22 and SEQ ID NO: 22, respectively; or
cc. SEQ ID NO: 368, SEQ ID NO: 369, SEQ ID NO: 22 and SEQ ID NO: 22, respectively; or
dd. SEQ ID NO: 390, SEQ ID NO: 389, SEQ ID NO: 10, and SEQ ID NO: 10, respectively; or
ee. SEQ ID NO: 382, SEQ ID NO: 389, SEQ ID NO: 10, and SEQ ID NO: 10, respectively; or
ff. SEQ ID NO: 382, SEQ ID NO: 385, SEQ ID NO: 10, and SEQ ID NO: 10, respectively; or
gg. SEQ ID NO: 382, SEQ ID NO: 385, SEQ ID NO: 11, and SEQ ID NO: 11, respectively; or
hh. SEQ ID NO: 383, SEQ ID NO: 389, SEQ ID NO: 10, and SEQ ID NO: 10, respectively; or
ii. SEQ ID NO: 383, SEQ ID NO: 386, SEQ ID NO: 10, and SEQ ID NO: 10, respectively; or
jj. SEQ ID NO: 383, SEQ ID NO: 386, SEQ ID NO: 12, and SEQ ID NO: 12, respectively; or
kk. SEQ ID NO: 384, SEQ ID NO: 389, SEQ ID NO: 10, and SEQ ID NO: 10, respectively; or
ll. SEQ ID NO: 384, SEQ ID NO: 387, SEQ ID NO: 10, and SEQ ID NO: 10, respectively; or
mm. SEQ ID NO: 382, SEQ ID NO: 388, SEQ ID NO: 10, and SEQ ID NO: 10, respectively; or
nn. SEQ ID NO: 382, SEQ ID NO: 388, SEQ ID NO: 11, and SEQ ID NO: 11, respectively; or
oo. SEQ ID NO: 382, SEQ ID NO: 386, SEQ ID NO: 10, and SEQ ID NO: 10, respectively; or
pp. SEQ ID NO: 384, SEQ ID NO: 386, SEQ ID NO: 10, and SEQ ID NO: 10, respectively; or
qq. SEQ ID NO: 382, SEQ ID NO: 386, SEQ ID NO: 12, and SEQ ID NO: 12, respectively; or
rr. SEQ ID NO: 384, SEQ ID NO: 386, SEQ ID NO: 12, and SEQ ID NO: 12, respectively; or
ss. SEQ ID NO: 382, SEQ ID NO: 387, SEQ ID NO: 10, and SEQ ID NO: 10, respectively; or
tt. SEQ ID NO: 383, SEQ ID NO: 387, SEQ ID NO: 10, and SEQ ID NO: 10, respectively; or
uu. SEQ ID NO: 383, SEQ ID NO: 387, SEQ ID NO: 11, and SEQ ID NO: 11, respectively; or
vv. SEQ ID NO: 384, SEQ ID NO: 387, SEQ ID NO: 11, and SEQ ID NO: 11, respectively; or
ww. SEQ ID NO: 384, SEQ ID NO: 385, SEQ ID NO: 11, and SEQ ID NO: 11, respectively; or
xx. SEQ ID NO: 370, SEQ ID NO: 371, SEQ ID NO: 10, and SEQ ID NO: 10, respectively; or
yy. SEQ ID NO: 372, SEQ ID NO: 373, SEQ ID NO: 10, and SEQ ID NO: 10, respectively; or
zz. SEQ ID NO: 374, SEQ ID NO: 375, SEQ ID NO: 10, and SEQ ID NO: 10, respectively or
aaa. SEQ ID NO: 376, SEQ ID NO: 377, SEQ ID NO: 10, and SEQ ID NO: 10, respectively; or
bbb. SEQ ID NO: 378, SEQ ID NO: 379, SEQ ID NO: 10, and SEQ ID NO: 10, respectively; or
ccc. SEQ ID NO: 380, SEQ ID NO: 381, SEQ ID NO: 10, and SEQ ID NO: 10, respectively.

In some embodiments, the gene editing system is a LigoRNA-based transformer base editor system. A transformer base editor (tBE) is a CRISPR-based gene editing system which can edit cytosine or adenosine in target regions with high specificity, preferably with no observable off-target mutations. In some embodiments, the transformer base editor (tBE) system comprises a CRISPR-associated protein (Cas protein) fused with a deaminase, a deaminase inhibitor domain, and a split-TEV protease (see FIG. 1). Thus, tBE remains inactive at off-target sites with a cleavable fusion of the deaminase inhibitor domain and eliminates unintended off-target mutations. Only when binding at on-target sites, tBE is transformed to cleave off the deaminase inhibitor domain and catalyzes targeted deamination for precise editing. A tBE system described by Wang et al. uses one main sgRNA (msgRNA) to bind at the target genomic site and one helper (hsgRNA) to bind at a nearby region (preferably upstream to the target genomic site). The binding of the two sgRNAs can guide the components of tBE system to correctly assemble at the target genomic site for base editing. However, due to the addition of protein-recruiting hairpins in hsgRNA and msgRNA, the length of these two sgRNA are over 100 nt. Chemically synthesized RNAs over 100 nt demonstrated much lower yield and purity, resulting in challenges for large-scale production and cost control. The LigoRNA-based tBE systems described herein do not require synthesis of long guide RNAs. They can be applied to perform highly precise and efficient base editing in various species.

In some embodiments of the gene editing system described herein, the gene editing system comprises
a. the hcrRNA comprising a first spacer sequence and a first linker sequence, wherein the first linker sequence comprises a first protein-binding motif,
b. the mcrRNA comprising a second spacer sequence and a second linker sequence, wherein the second linker sequence comprises a second protein-binding motif,
c. a first tracrRNA which is capable of forming a first base-pair structure with the hcrRNA,
d. a second tracrRNA which is capable of forming a second base-pair structure with the mcrRNA,
e. a first CRISPR-associated protein (Cas protein), or a polynucleotide encoding the first Cas protein, wherein the first Cas protein binds to the first base-pair structure,
f. a second Cas protein, or a polynucleotide encoding the second Cas protein, wherein the second Cas protein binds to the second base pair structure,
g. a first fusion protein comprising a nucleobase deaminase or a catalytic domain thereof and a first RNA binding domain, or a polynucleotide encoding the first fusion protein, wherein the nucleobase deaminase or the catalytic domain thereof and the first RNA binding domain are optionally connected by a linker, and wherein the first RNA binding domain binds to the first protein-binding motif,
wherein the first Cas protein and the second Cas protein are the same or different, and the first tracrRNA and the second tracrRNA are the same or different.

In some embodiments of the gene editing system described herein, it further comprises
a. a protease, or a polynucleotide encoding the protease, and
b. a nucleobase deaminase inhibitor domain,
wherein the nucleobase deaminase inhibitor domain is connected to the nucleobase deaminase or the catalytic domain thereof in the first fusion protein optionally by a linker, and wherein there is a cleavage site for the protease between the nucleobase deaminase inhibitor domain and the nucleobase deaminase or the catalytic domain thereof.

In some embodiments of the gene editing system described herein, the gene editing system comprises
a. the hcrRNA comprising a first spacer sequence and a first linker sequence, wherein the first linker sequence comprises a first protein-binding motif,
b. the mcrRNA comprising a second spacer sequence and a second linker sequence, wherein the second linker sequence comprises a second protein-binding motif,
c. a first tracrRNA which is capable of forming a first base-pair structure with the hcrRNA,
d. a second tracrRNA which is capable of forming a second base-pair structure with the mcrRNA,
e. a first CRISPR-associated protein (Cas protein), or a polynucleotide encoding the first Cas protein, wherein the first Cas protein binds to the first base-pair structure,
f. a second Cas protein, or a polynucleotide encoding the second Cas protein, wherein the second Cas protein binds to the second base pair structure,
g. a first fusion protein comprising a nucleobase deaminase or a catalytic domain thereof and a first RNA binding domain, or a polynucleotide encoding the first fusion protein, wherein the nucleobase deaminase or the catalytic domain thereof and the first RNA binding domain are optionally connected by a linker, and wherein the first RNA binding domain binds to the first protein-binding motif,
h. a protease, or a polynucleotide encoding the protease,
i. a nucleobase deaminase inhibitor domain, and
j. a second fusion protein comprising the protease and a second RNA binding domain, or a polynucleotide encoding the second fusion protein,
wherein the first Cas protein and the second Cas protein are the same or different, and the first tracrRNA and the second tracrRNA are the same or different,
wherein the nucleobase deaminase inhibitor domain is connected to the nucleobase deaminase or the catalytic domain thereof in the first fusion protein optionally by a linker, and wherein there is a cleavage site for the protease between the nucleobase deaminase inhibitor domain and the nucleobase deaminase or the catalytic domain thereof,
wherein the protease and the second RNA binding domain are optionally connected by a linker, and
wherein the second RNA binding domain binds to the second protein-binding motif.

In some embodiments of the gene editing system described herein, the protease is split into a first protease fragment and a second protease fragment, wherein the first and/or second protease fragment alone is not able to cleave the cleavage site.

In some embodiments of the gene editing system described herein, wherein the gene editing system comprises
a. the hcrRNA comprising a first spacer sequence and a first linker sequence, wherein the first linker sequence comprises a first protein-binding motif,
b. the mcrRNA comprising a second spacer sequence and a second linker sequence, wherein the second linker sequence comprises a second protein-binding motif,
c. a first tracrRNA which is capable of forming a first base-pair structure with the hcrRNA,
d. a second tracrRNA which is capable of forming a second base-pair structure with the mcrRNA,
e. a first CRISPR-associated protein (Cas protein), or a polynucleotide encoding the first Cas protein, wherein the first Cas protein binds to the first base-pair structure,
f. a second Cas protein, or a polynucleotide encoding the second Cas protein, wherein the second Cas protein binds to the second base pair structure,
g. a first fusion protein comprising a nucleobase deaminase or a catalytic domain thereof and a first RNA binding domain, or a polynucleotide encoding the first fusion protein, wherein the nucleobase deaminase or the catalytic domain thereof and the first RNA binding domain are optionally connected by a linker, and wherein the first RNA binding domain binds to the first protein-binding motif,
h. a protease, or a polynucleotide encoding the protease, wherein the protease is split into a first protease fragment and a second protease fragment, wherein the first and/or second protease fragment alone is not able to cleave the cleavage site,
i. a nucleobase deaminase inhibitor domain,
j. a second fusion protein comprising the first protease fragment and a second RNA binding domain, or a polynucleotide encoding the second fusion protein, wherein the first protease fragment and the second RNA binding domain are optionally connected by a linker, and
k. a third fusion protein comprising the second protease fragment and a third RNA binding domain, or a polynucleotide encoding the third fusion protein, wherein the second protease fragment and the third RNA binding domain are optionally connected by a linker,
wherein the first Cas protein and the second Cas protein are the same or different, and the first tracrRNA and the second tracrRNA are the same or different,
wherein the nucleobase deaminase inhibitor domain is connected to the nucleobase deaminase or the catalytic domain thereof in the first fusion protein optionally by a linker, and wherein there is a cleavage site for the protease between the nucleobase deaminase inhibitor domain and the nucleobase deaminase or the catalytic domain thereof,
wherein the mcrRNA further comprises a third protein-binding motif,
wherein the second RNA binding domain binds to the second protein-binding motif, and
wherein the third RNA binding domain binds to the third protein-binding motif.

In some embodiments of the gene editing system described herein, the gene editing system comprises
a. the hcrRNA comprising a first spacer sequence and a first linker sequence, wherein the first linker sequence comprises a first protein-binding motif,
b. the mcrRNA comprising a second spacer sequence and a second linker sequence, wherein the second linker sequence comprises a second protein-binding motif,
c. a first tracrRNA which is capable of forming a first base-pair structure with the hcrRNA,
d. a second tracrRNA which is capable of forming a second base-pair structure with the mcrRNA,
e. a first CRISPR-associated protein (Cas protein), or a polynucleotide encoding the first Cas protein, wherein the first Cas protein binds to the first base-pair structure,
f. a second Cas protein, or a polynucleotide encoding the second Cas protein, wherein the second Cas protein binds to the second base pair structure,
g. a first fusion protein comprising a nucleobase deaminase or a catalytic domain thereof and a first RNA binding domain, or a polynucleotide encoding the first fusion protein, wherein the nucleobase deaminase or the catalytic domain thereof and the first RNA binding domain are optionally connected by a linker, and wherein the first RNA binding domain binds to the first protein-binding motif,
h. a protease, or a polynucleotide encoding the protease, wherein the protease is split into a first protease fragment and a second protease fragment, wherein the first and/or second protease fragment alone is not able to cleave the cleavage site,
i. a nucleobase deaminase inhibitor domain,
j. a second fusion protein comprising the first protease fragment and a second RNA binding domain, or a polynucleotide encoding the second fusion protein, wherein the first protease fragment and the second RNA binding domain are optionally connected by a linker, and
k. a third fusion protein comprising the second protease fragment and a third RNA binding domain, or a polynucleotide encoding the third fusion protein, wherein the second protease fragment and the third RNA binding domain are optionally connected by a linker,
wherein the first Cas protein and the second Cas protein are the same or different, and the first tracrRNA and the second tracrRNA are the same or different,
wherein the nucleobase deaminase inhibitor domain is connected to the nucleobase deaminase or the catalytic domain thereof in the first fusion protein optionally by a linker, and wherein there is a cleavage site for the protease between the nucleobase deaminase inhibitor domain and the nucleobase deaminase or the catalytic domain thereof,
wherein the mcrRNA further comprises a third protein-binding motif,
wherein the second RNA binding domain binds to the second protein-binding motif,
wherein the third RNA binding domain binds to the third protein-binding motif, and
wherein the second and the third RNA binding domains are the same or different, and the second and the third protein-binding motifs are the same or different.

In some embodiments of the gene editing system described herein, the gene editing system comprises
a. the hcrRNA comprising a first spacer sequence and a first linker sequence, wherein the first linker sequence comprises a first protein-binding motif,
b. the mcrRNA comprising a second spacer sequence and a second linker sequence, wherein the second linker sequence comprises a second protein-binding motif,
c. a first tracrRNA which is capable of forming a first base-pair structure with the hcrRNA,
d. a second tracrRNA which is capable of forming a second base-pair structure with the mcrRNA,
e. a first CRISPR-associated protein (Cas protein), or a polynucleotide encoding the first Cas protein, wherein the first Cas protein binds to the first base-pair structure,
f. a second Cas protein, or a polynucleotide encoding the second Cas protein, wherein the second Cas protein binds to the second base pair structure,
g. a first fusion protein comprising a nucleobase deaminase or a catalytic domain thereof and a first RNA binding domain, or a polynucleotide encoding the first fusion protein, wherein the nucleobase deaminase or the catalytic domain thereof and the first RNA binding domain are optionally connected by a linker, and wherein the first RNA binding domain binds to the first protein-binding motif,
h. a protease, or a polynucleotide encoding the protease, wherein the protease is split into a first protease fragment and a second protease fragment, wherein the first and/or second protease fragment alone is not able to cleave the cleavage site,
i. a nucleobase deaminase inhibitor domain,
j. a second fusion protein comprising the first protease fragment and a second RNA binding domain, or a polynucleotide encoding the second fusion protein,
wherein the first Cas protein and the second Cas protein are the same or different, and the first tracrRNA and the second tracrRNA are the same or different,
wherein the nucleobase deaminase inhibitor domain is connected to the nucleobase deaminase or the catalytic domain thereof in the first fusion protein optionally by a linker, and wherein there is a cleavage site for the protease between the nucleobase deaminase inhibitor domain and the nucleobase deaminase or the catalytic domain thereof,
wherein the first protease fragment and the second RNA binding domain are optionally connected by a linker, and
wherein the second RNA binding domain binds to the second protein-binding motif.

In some embodiments of the gene editing system described herein, the protease is a TEV protease, a TuMV protease, a PPV protease, a PVY protease, a ZIKV protease, or a WNV protease.

A “protease” refers to an enzyme that catalyzes proteolysis. A “cleavage site for a protease” refers to a short peptide that the protease recognizes, and within the short peptide creates a proteolytic cleavage. Non-limiting examples of proteases include TEV protease, TuMV protease, PPV protease, PVY protease, ZIKV protease, and WNV protease. The protein sequences of example proteases and their corresponding cleavage sites are provided in Table 2.

TABLE 2
Exemplary proteases and their cleavage sites

| Name | Sequence | SEQ ID NO |
|---|---|---|
| TEV protease | MGESLFKGPRDYNPISSTICHLTNESDGHTTSL | SEQ ID NO: 25 |
| | YGIGFGPFIITNKHLFRRNNGTLLVQSLHGVFK | |
| | VKNTTTLQQHLIDGRDMIIIRMPKDFPPFPQKL | |
| | KFREPQREERICLVTTNFQTKSMSSMVSDTSCT | |
| | FPSSDGIFWKHWIQTKDGQCGSPLVSTRDGFIV | |
| | GIHSASNFTNTNNYFTSVPKNFMELLTNQEAQ | |
| | QWVSGWRLNADSVLWGGHKVFMVKPEEPFQ | |
| | PVKEATQLMN | |
| TEV protease N- | MGESLFKGPRDYNPISSTICHLTNESDGHTTSL | SEQ ID NO: 26 |
| terminal domain | YGIGFGPFIITNKHLFRRNNGTLLVQSLHGVFK | |
| | VKNTTTLQQHLIDGRDMIIIRMPKDFPPFPQKL | |
| | KFREPQREERICLVTTNFQT | |
| TEV protease C- | MKSMSSMVSDTSCTFPSSDGIFWKHWIQTKD | SEQ ID NO: 27 |
| terminal domain | GQCGSPLVSTRDGFIVGIHSASNFTNTNNYFTS | |
| | VPKNFMELLTNQEAQQWVSGWRLNADSVLW | |
| | GGHKVFMVKPEEPFQPVKEATQ | |
| TEV protease | ENLYFQS | SEQ ID NO: 28 |
| cleavage site | | |
| TuMV protease | MASSNSMFRGLRDYNPISNNICHLTNVSDGAS | SEQ ID NO: 29 |
| | NSLYGVGFGPLILTNRHLFERNNGELVIKSRHG | |
| | EFVIKNTTQLHLLPIPDRDLLLIRLPKDVPPFPQ | |
| | KLGFRQPEKGERICMVGSNFQTKSITSIVSETST | |
| | IMPVENSQFWKHWISTKDGQCGSPMVSTKDG | |
| | KILGLHSLANFQNSINYFAAFPDDFAEKYLHTI | |
| | EAHEWVKHWKYNTSAISWGSLNIQASQPSGL | |
| | FKVSKLISDLDSTAVYAQ | |
| TuMV protease | GGCSHQS | SEQ ID NO: 30 |
| cleavage site | | |
| PPV protease | MASSKSLFRGLRDYNPIASSICQLNNSSGARQS | SEQ ID NO: 31 |
| | EMFGLGFGGLIVTNQHLFKRNDGELTIRSHHG | |
| | EFVVKDTKTLKLLPCKGRDIVIIRLPKDFPPFPR | |
| | RLQFRTPTTEDRVCLIGSNFQTKSISSTMSETSA | |
| | TYPVDNSHFWKHWISTKDGHCGLPIVSTRDGS | |
| | ILGLHSLANSTNTQNFYAAFPDNFETTYLSNQ | |
| | DNDNWIKQWRYNPDEVCWGSLQLKRDIPQSP | |
| | FTICKLLTDLDGEFVYTQ | |
| PPV protease | QVVVHQSK | SEQ ID NO: 32 |
| cleavage site | | |
| PVY protease | MASAKSLMRGLRDFNPIAQTVCRLKVSVEYG | SEQ ID NO: 33 |
| | ASEMYGFGFGAYIVANHHLFRSYNGSMEVQS | |
| | MHGTFRVKNLHSLSVLPIKGRDIILIKMPKDFP | |
| | VFPQKLHFRAPTQNERICLVGTNFQEKYASSII | |
| | TETSTTYNIPGSTFWKHWIETDNGHCGLPVVS | |
| | TADGCIVGIHSLANNAHTTNYYSAFDEDFESK | |
| | YLRTNEHNEWVKSWVYNPDTVLWGPLKLKD | |
| | STPKGLFKTTKLVQDLIDHDVVVEQ | |
| PVY protease | YDVRHQSR | SEQ ID NO: 34 |
| cleavage site | | |
| ZIKV protease | MASDMYIERAGDITWEKDAEVTGNSPRLDVA | SEQ ID NO: 35 |
| | LDESGDFSLVEEDGPPMREGGGGSGGGGSGA | |
| | LWDVPAPKEVKKGETTDGVYRVMTRRLLGST | |
| | QVGVGVMQEGVFHTMWHVTKGAALRSGEGR | |
| | LDPYWGDVKQDLVSYCGPWKLDAAWDGLSE | |
| | VQLLAVPPGERARNIQTLPGIFKTKDGDIGAVA | |
| | LDYPAGTSGSPILDKCGRVIGLYGNGVVIKNG | |
| | SYVSAITQGKREEETPVECFE | |
| ZIKV protease | KERKRRGA | SEQ ID NO: 36 |
| cleavage site | | |
| WNV protease | MASSTDMWIERTADISWESDAEITGSSERVDV | SEQ ID NO: 37 |
| | RLDDDGNFQLMNDPGAPWKGGGGSGGGGGV | |
| | LWDTPSPKEYKKGDTTTGVYRIMTRGLLGSY | |
| | QAGAGVMVEGVFHTLWHTTKGAALMSGEGR | |
| | LDPYWGSVKEDRLCYGGPWKLQHKWNGQDE | |
| | VQMIVVEPGKNVKNVQTKPGVFKTPEGEIGA | |
| | VTLDFPTGTSGSPIVDKNGDVIGLYGNGVIMP | |
| | NGSYISAIVQGERMDEPIPAGFEPEML | |
| WNV protease | KQKKRGGK | SEQ ID NO: 38 |
| cleavage site | | |

In some embodiments, the protease cleavage site is a self-cleaving peptide, such as the 2A peptides. “2A peptides” are 18-22 amino-acid-long viral oligopeptides that mediate “cleavage” of polypeptides during translation in eukaryotic cells. The designation “2A” refers to a specific region of the viral genome and different viral 2As have generally been named after the virus they were derived from. The first discovered 2A was F2A (foot-and-mouth disease virus), after which E2A (equine rhinitis A virus), P2A (porcine teschovirus-1 2A), and T2A (thosea asigna virus 2A) were also identified. A few non-limiting examples of 2A peptides are provided in SEQ ID NOs: 219-221.

In some embodiments, the first and/or the second TEV protease fragment is not able to cleave the TEV cleavage site on its own. However, in the presence of the remaining portion of the TEV protease, this fragment will be able to effectuate the cleavage. The TEV fragment may be the TEV N-terminal domain (e.g., SEQ ID NO: 26) or the TEV C-terminal domain (e.g., SEQ ID NO: 27). In some embodiments, the first TEV protease fragment comprises a sequence of SEQ ID NO: 26. In some embodiments, the first TEV protease fragment comprises a sequence of SEQ ID NO: 27.

In some embodiments of the gene editing system described herein, the protease is a TEV protease comprising a sequence of SEQ ID NO: 25.

In some embodiments of the gene editing system described herein, the first TEV protease fragment comprises a sequence of SEQ ID NO: 26.

In some embodiments of the gene editing system described herein, the nucleobase deaminase inhibitor is an inhibitory domain of a nucleobase deaminase. A “nucleobase deaminase inhibitor” or an “inhibitory domain” refers to a protein or a protein domain that inhibits the deaminase activity of a nucleobase deaminase.

In some embodiments of the gene editing system described herein, the nucleobase deaminase inhibitor is an inhibitory domain of a cytidine deaminase and/or an adenosine deaminase.

In some embodiments of the gene editing system described herein, the inhibitory domain comprises an amino acid sequence of any one of SEQ ID NOs: 42-43 and 51-138.

In some embodiments of the gene editing system described herein, the nucleotide deaminase is a cytidine deaminase.

“Cytidine deaminase” refers to enzymes that catalyze the hydrolytic deamination of cytidine and deoxycytidine to uridine and deoxyuridine, respectively. Cytidine deaminases maintain the cellular pyrimidine pool. A family of cytidine deaminases is APOBEC (“apolipoprotein B mRNA editing enzyme, catalytic polypeptide-like”). Members of this family are C-to-U editing enzymes. Some APOBEC family members have two domains, one domain of APOBEC like proteins is the catalytic domain, while the other domain is a pseudocatalytic domain. More specifically, the catalytic domain is a zinc dependent cytidine deaminase domain and is important for cytidine deamination. RNA editing by APOBEC-1 requires homodimerisation and this complex interacts with RNA binding proteins to form the editosome.

Non-limiting examples of APOBEC proteins include APOBEC1, APOBEC2, APOBEC3A, APOBEC3B, APOBEC3C, APOBEC3D, APOBEC3F, APOBEC3G, APOBEC3H, APOBEC4, and activation-induced (cytidine) deaminase (AID).

Various mutants of the APOBEC proteins are also known that have brought about different editing characteristics for base editors. For instance, for human APOBEC3A, certain mutants (e.g., W98Y, Y130F, Y132D, W104A, D131Y and P134Y) even outperform the wildtype human APOBEC3A in terms of editing efficiency or editing window. Accordingly, the term APOBEC and each of its family member also encompasses variants and mutants that have certain level (e.g., 70%, 75%, 80%, 85%, 90%, 95%, 98%, 99%) of sequence identity to the corresponding wildtype APOBEC protein or the catalytic domain and retain the cytidine deaminating activity. The variants and mutants can be derived with amino acid additions, deletions and/or substitutions. Such substitutions, in some embodiments, are conservative substitutions.

In some embodiments of the gene editing system described herein, the cytidine deaminase is selected from the group consisting of APOBEC3A (A3A), APOBEC3B (A3B), APOBEC3C (A3C), APOBEC3D (A3D), APOBEC3F (A3F), APOBEC3G (A3G), APOBEC3H (A3H), APOBEC1 (A1), APOBEC3 (A3), APOBEC2 (A2), APOBEC4 (A4), and AICDA (AID).

In some embodiments of the gene editing system described herein, the cytidine deaminase comprises an amino acid sequence of SEQ ID NO: 252-287.

In some embodiments of the gene editing system described herein, the cytidine deaminase is a naturally occurring cytidine deaminase, an engineered cytidine deaminase, an evolved cytidine deaminase, or an adenosine deaminase that possesses cytidine deaminase activity.

In some embodiments of the gene editing system described herein, the cytidine deaminase is a human or mouse cytidine deaminase.

In some embodiments of the gene editing system described herein, the catalytic domain of the cytidine deaminase is a mouse A3 cytidine deaminase domain 1 (mA3-CDA1) or human A3B cytidine deaminase domain 2 (hA3B-CDA2).

Table 3 shows 44 proteins/domains that have significant sequence homology to mA3-CDA2 core sequence and Table 4 shows 43 proteins/domains that have significant sequence homology to hA3B-CDA1. All of these proteins and domains, as well as their variants and equivalents, are contemplated to have nucleobase deaminase inhibition activities.

TABLE 3

| Name | Sequence | SEQ ID NO: |
|---|---|---|
| Mouse APOBEC3 cytidine | SEKGKQHAEILFLDKIRSMELSQVTITCYL | SEQ ID NO: 51 |
| deaminase domain 2 core | TWSPCPNCAWQLAAFKRDRPDLILHIYTS | |
| (AA282-AA355) | RLYFHWKRPFQKGLC | |
| Mus spicilegus A3 | SEKGKQHAEILFLDKIRSMELSQVTITCYL | SEQ ID NO: 52 |
| (AA248-AA321) | TWSPCPNCAWQLAAFKRDRPDLIPHIYTSR | |
| | LYFHWKRPFQKGLC | |
| Cricetulus longicaudatus | SEKGKQHAEILFLDKIRSMELSQVTITCYL | SEQ ID NO: 53 |
| A3 (AA249-AA322) | TWSPCPNCAWRLAAFKRDRPDLILHIYTSR | |
| | LYFHWKRPFQKGLC | |
| Mus terricolor A3 (AA248- | SEKGKQHAEILFLNKIRSMELSQVTITCYL | SEQ ID NO: 54 |
| AA321) | TWSPCPNCAWQLAAFKKDRPDLILHIYTS | |
| | RLYFHWKRPFQKGLC | |
| Mus caroli A3 (AA260- | SKKGKQHAEILFLDKIRSMELSQVTITCYL | SEQ ID NO: 55 |
| AA333) | TWSPCPNCAWQLAAFKRDHPDLILHIYTS | |
| | RLYFHWKRPFQKGLC | |
| Mus pahari A3 (AA263- | SKKGKQHAEILFLEKIRSMELSQMRITCYL | SEQ ID NO: 56 |
| AA336) | TWSPCPNCAWQLAAFQKDRPDLILHIYTS | |
| | RLYFHWRRIFQKGLC | |
| Mus shortridgei A3 | SKKGKQHAEILFLEKIRSMELSQMRITCYL | SEQ ID NO: 57 |
| (AA233-AA306) | TWSPCPNCAWQLAAFQKDRPDLILHIYTS | |
| | RLYFHWRRIFQKGLC | |
| Mus setulosus A3 (AA29- | SKKGKQHAEILFLDKIRSMELSQVRITCYL | SEQ ID NO: 58 |
| AA302) | TWSPCPNCAWQLETFKKDRPDLILHIYTSR | |
| | LYFHWKRAFQEGLC | |
| Grammomys surdaster A3 | SKKGKPHAEILFLDKMWSMEELSQVRITC | SEQ ID NO: 59 |
| (AA270-AA344) | YLTWSPCPNCARQLAAFKKDHPGLILRIYT | |
| | SRLYFYWRRKFQKGLC | |
| Rattus norvegicus A3 | KKGEQHVEILFLEKMRSMELSQVRITCYLT | SEQ ID NO: 60 |
| (AA256-AA328) | WSPCPNCARQLAAFKKDHPDLILRIYTSRL | |
| | YFYWRKKFQKGLC | |
| Mastomys coucha A3 | SKKGRQHAEILFLEKVRSMQLSQVRITCYL | SEQ ID NO: 61 |
| (AA258-AA331) | TWSPCPNCAWQLAAFKMDHPDLILRIYAS | |
| | RLYFHWRRAFQKGLC | |
| Cricetulus griseus A3B | NKKGKHAEILFIDEMRSLELGQVQITCYLT | SEQ ID NO: 62 |
| (AA235-AA307) | WSPCPNCAQELAAFKSDHPDLVLRIYTSRL | |
| | YFHWRRKYQEGLC | |
| Peromyscus leucopus A3 | NKKGKHAEILFIDEMRSLELGQARITCYLT | SEQ ID NO: 63 |
| (AA266-AA338) | WSPCPNCAQKLAAFKKDHPDLVLRVYTS | |
| | RLYFHWRRKYQEGLC | |
| Mesocricetus auratus A3 | NKKDKHAEILFIDKMRSLELCQVRITCYLT | SEQ ID NO: 64 |
| (AA268-AA340) | WSPCPNCAQELAAFKKDHPDLVLRIYTSR | |
| | LYFHWRRKYQEGLC | |
| Microtus ochrogaster A3B | NKKGKHAEILFIDEMRSLKLSQERITCYLT | SEQ ID NO: 65 |
| (AA266-AA338) | WSPCPNCAQELAAFKRDHPGLVLRIYASR | |
| | LYFHWRRKYQEGLC | |
| Nannospalax galili A3 | NKRAKHAEILLIDMMRSMELGQVQITCYI | SEQ ID NO: 66 |
| (AA231-AA302) | TWSPCPTCAQELAAFKQDHPDLVLRIYAS | |
| | RLYFHWKRKFQKGL | |
| Meriones unguiculatus A3 | NKKGRHAEICLIDEMRSLGLGKAQITCYLT | SEQ ID NO: 67 |
| (AA233-AA305) | WSPCRKCAQELATFKKDHPDLVLRVYAS | |
| | RLYFHWSRKYQQGLC | |
| Dipodomys ordii A3 | NKKGHHAEIRFIERIRSMGLDPSQDYQITC | SEQ ID NO: 68 |
| (AA256-AA330) | YLTWSPCLDCAFKLAKLKKDFPRLTLRIFT | |
| | SRLYFHWIRKFQKGL | |
| Jaculus jaculus A3 | NKKGKHAEARFVDKMRSMQLDHALITCY | SEQ ID NO: 69 |
| (AA303-AA374) | LTWSPCLDCSQKLAALKRDHPGLTLRIFTS | |
| | RLYFHWVKKFQEGL | |
| Chinchilla lanigera A3H | SPQKGHHAESRFIKRISSMDLDRSRSYQITC | SEQ ID NO: 70 |
| (AA86-AA161) | FLTWSPCPSCAQELASFKRAHPHLRFQIFV | |
| | SRLYFHWKRSYQAGL | |
| Heterocephalus glaber A3 | KKGYHAESRFIKRICSMDLGQDQSYQVTC | SEQ ID NO: 71 |
| (AA277-AA350) | FLTWSPCPHCAQELVSFKRAHPHLRLQIFT | |
| | ARLFFHWKRSYQEGL | |
| Octodon degus A3 | KKGQHAEIRFIERIHSMALDQARSYQITCF | SEQ ID NO: 72 |
| (AA256-AA329) | LTWSPCPFCAQELASFKSTHPRVHLQIFVS | |
| | RLYFHWKRSYQEGL | |
| Urocitellus parryii A3 | NKKGHHAEIRFIKKIRSLDLDQSQNYEVTC | SEQ ID NO: 73 |
| (AA256-AA330) | YLTWSPCPDCAQELVALTRSHPHVRLRLF | |
| | TSRLYFHWFWSFQEGL | |
| Aotus nancymaae A3H | NRHAEICFIDEIESMGLDKTQCYEVTCYLT | SEQ ID NO: 74 |
| (AA75-AA146) | WSPCPSCAQKLAAFTKAQVHLNLRIFASR | |
| | LYYHWRSSYQKGL | |
| Cebus capucinus imitator | NRHAEICFIDEIESMGLDKTQCYEVTCYLT | SEQ ID NO: 75 |
| A3H (AA55-AA126) | WSPCPSCAQKLVAFAKAQDHLNLRIFASR | |
| | LYYHWRRRYKEGL | |
| Saimiri boliviensis | HVEICFIDKIASMELDKTQCYDVTCYLTWS | SEQ ID NO: 76 |
| boliviensis A3H (AA56- | PCPSCAQKLAAFAKAQDHLNLRIFASRLY | |
| AA125) | YHWRRSYQKGL | |
| Homo sapiens A3H | NKKKCHAEICFINEIKSMGLDETQCYQVTC | SEQ ID NO: 77 |
| (AA49-AA123) | YLTWSPCSSCAWELVDFIKAHDHLNLGIF | |
| | ASRLYYHWCKPQQKGL | |
| Homo sapiens ARP10 | ENKKKCHAEICFINEIKSMGLDETQCYQVT | SEQ ID NO: 78 |
| (AA48-AA123) | CYLTWSPCSSCAWELVDFIKAHDHLNLGI | |
| | FASRLYYHWCKPQQKGL | |
| Pan paniscus A3H (AA49- | NKKKCHAEICFINEIKSMGLDETQCYQVTC | SEQ ID NO: 79 |
| AA123) | YLTWSPCSSCAWKLVDFIQAHDHLNLRIF | |
| | ASRLYYHWCKPQQEGL | |
| Symphalangus syndactylus | NKKKRHAEIRFINKIKSMGLDETQCYQVT | SEQ ID NO: 80 |
| A3H (AA49-AA123) | CYLTWSPCPSCAWELVDFIKAHDHLNLGI | |
| | FASRLYYHWCRHQQEGL | |
| Macaca mulatta A3H | NKKKDHAEIRFINKIKSMGLDETQCYQVT | SEQ ID NO: 81 |
| (AA49-AA123) | CYLTWSPCPSCAGELVDFIKAHRHLNLRIF | |
| | ASRLYYHWRPNYQEGL | |
| Theropithecus gelada A3H | NKKKEHAEIRFINKIKSMGLDETQCYQVT | SEQ ID NO: 82 |
| (AA54-AA128) | CYLTWSPCPSCAGKLVDFIKAHHHLNLRIF | |
| | ASRLYYHWRPNYQEGL | |
| Mandrillus leucophaeus | NKKKHHAEIHFINKIKSMGLDETQCYQVT | SEQ ID NO: 83 |
| A3H (AA49-AA123) | CYLTWSPCPSCARELVDFIKAHRHLNLRIF | |
| | ASRLYYHWRPHYQEGL | |
| Bos grunniens A3 | NKKQRHAEIRFIDKINSLDLNPSQSYKIICYI | SEQ ID NO: 84 |
| (AA74-AA148) | TWSPCPNCANELVNFITRNNHLKLEIFASR | |
| | LYFHWIKPFKMGL | |
| Bubalus bubalis A3 | NKKQRHAEIRFIDKINSLDLNPSQSYKIICYI | SEQ ID NO: 85 |
| (AA74-AA148) | TWSPCPNCASELVDFITRNDHLDLQIFASR | |
| | LYFHWIKPFKRGL | |
| Odocoileus virginianus | NKKQRHAEIRFIDKINSLNLDRRQSYKIICY | SEQ ID NO: 86 |
| texanus A3H (AA209- | ITWSPCPRCASELVDFITGNDHLNLQIFASR | |
| AA283) | LYFHWKKPFQRGL | |
| Sus scrofa A3 (AA51- | NKKKRHAEIRFIDKINSLNLDQNQCYRIIC | SEQ ID NO: 87 |
| AA125) | YVTWSPCHNCAKELVDFISNRHHLSLQLF | |
| | ASRLYFHWVRCYQRGL | |
| Ceratotherium simum | NKKKRHAEIRFIDKIKSLGLDRVQSYEITC | SEQ ID NO: 88 |
| simum A3B (AA232- | YITWSPCPTCALELVAFTRDYPRLSLQIFAS | |
| AA306) | RLYFHWRRRSIQGL | |
| Equus caballus A3H | NKKKRHAEIRFIDKINSLGLDQDQSYEITC | SEQ ID NO: 89 |
| (AA79-AA153) | YVTWSPCATCACKLIKFTRKFPNLSLRIFV | |
| | SRLYYHWFRQNQQGL | |
| Enhydra lutris kenyoni | KKKRHAEIRFIDSIRALQLDQSQRFEITCYL | SEQ ID NO: 90 |
| A3B (AA243-AA316) | TWSPCPTCAKELAMFVQDHPHISLRLFASR | |
| | LYFHWRWKYQEGL | |
| Leptonychotes weddellii | KKKRHAEIRFIDNIKALRLDTSQRFEITCYV | SEQ ID NO: 91 |
| A3H (AA50-AA123) | TWSPCPTCAKELVAFVRDHRHISLRLFASR | |
| | LYFHWLRENKKGL | |
| Ursus arctos horribilis A3F | NKKKRHAEIRFIDKIRSLQRDSSQTFEITCY | SEQ ID NO: 92 |
| (AA552-AA626) | VTWSPCFTCAEELVAFVRDHPHVRLRLFA | |
| | SRLYFHWLRKYQEGL | |
| Panthera leo bleyenberghi | NKKKRHAEICFIDKIKSLTRDTSQRFEIICYI | SEQ ID NO: 93 |
| A3H (AA50-AA124) | TWSPCPFCAEELVAFVKDNPHLSLRIFASR | |
| | LYVHWRWKYQQGL | |
| Panthera tigris sumatrae | NKKKRHAEICFIDKIKSLTRDTSQRFEIICYI | SEQ ID NO: 94 |
| A3H (AA50-AA124) | TWSPCPFCAEELVAFVKDNPHLSLRIFASR | |
| | LYVHWRWKYQQGL | |
| Tupaia belangeri A3 | NKKHRHAEVRFIAKIRSMSLDLDQKHQLT | SEQ ID NO: 95 |
| (AA46-AA120) | CYLTWSPCPSCAQELVTFMAESRHLNLQV | |
| | FVSRLYFHWQRDFQQGL | |

TABLE 4

| Name | Sequence | SEQ ID NO: |
|---|---|---|
| Gorilla A3B | GRSYNWLCYEVKIKRGRSNLLWNTGVFRGQMYS | SEQ ID NO: 96 |
| (AA29-AA138) | QPEHHAEMCFLSWFCGNQLPAYKCFQITWFVSW | |
| | TPCPDCVAKLAEFLAEYPNVTLTISTARLYYYWE | |
| | RDYRRALCRL | |
| Pan paniscus A3B | GRSYTWLCYEVKIRRGHSNLLWDTGVFRGQMYS | SEQ ID NO: 97 |
| (AA29-AA138) | QPEHHAEMYFLSWFCGNQLPAYKCFQITWFVSW | |
| | TPCPDCVAKLAEFLAEHPNVTLTISAARLYYYWE | |
| | RDYRRALCRL | |
| Pan troglodytes | GRSYTWLCYEVKIRRGHSNLLWDTGVFRGQMYS | SEQ ID NO: 98 |
| A3B (AA29- | QPEHHAEMCFLSWFCGNQLSAYKCFQITWFVSW | |
| AA138) | TPCPDCVAKLAKFLAEHPNVTLTISAARLYYYWE | |
| | RDYRRALCRL | |
| Gorilla A3F | RNTVWLCYEVKTKGPSRPPLDAKIFRGQVYFEPQ | SEQ ID NO: 99 |
| (AA30-AA137) | YHAEMCFLSWFCGNQLPAYKCFQITWFVSWTPCP | |
| | DCVAKLAEFLAEHPNVTLTISAARLYYYWE | |
| Pan troglodytes | RNTVWLCYEVKTKGPSRPRLDTKIFRGQVYFEPQ | SEQ ID NO: 100 |
| A3F (AA30- | YHAEMCFLSWFCGNQLPAYKCFQITWFVSWTPCP | |
| AA137) | DCVAKLAEFLAEHPNVTLTISAARLYYYWERDYR | |
| | RALCRL | |
| Human sapiens | RNTVWLCYEVKTKGPSRPRLDAKIFRGQVYSQPE | SEQ ID NO: 101 |
| A3F (AA30- | HHAEMCFLSWFCGNQLPAYKCFQITWFVSWTPCP | |
| AA137) | DCVAKLAEFLAEHPNVTLTISAARLYYYWERDYR | |
| | RALCRL | |
| Macaca leonine | RNTVWLCYEVKTRGPSMPTWGTKIFRGQVCFEPQ | SEQ ID NO: 102 |
| A3F (AA30- | YHAEMCFLSRFCGNQLPAYKRFQITWFVSWTPCP | |
| AA137) | DCVAKVAEFLAEHPNVTLTISAARLYYYWETDYR | |
| | RALCRL | |
| Macaca | RNTVWLCYEVKTRGPSMPTWGTKIFRGQVCFEPQ | SEQ ID NO: 103 |
| nemestrina A3F | YHAEMCFLSRFCGNQLPAYKRFQITWFVSWTPCP | |
| (AA30-AA137) | DCVAKVAEFLAEHPNVTLTISAARLYYYWETDYR | |
| | RALCRL | |
| Rhinopithecus | RNTVWLCYEVKTRGPSMPTWGAKIFRGQVYFEP | SEQ ID NO: 104 |
| roxellana A3F | QYHAEMCFLSWFCGNQLPAYKRFQITWFVSWTP | |
| (AA30-AA137) | CPDCVAKVAEFLAEHPNVTLTISAARLYYYWETD | |
| | YRRALCRL | |
| Mandrillus | RNTVWLCYKVKTRGPSMPTWGTKIFRGQVYFQP | SEQ ID NO: 105 |
| leucophaeus A3F | QYHAEMCFLSWFCGNQLPAYKRFQITWFVSWTP | |
| (AA30-AA130) | CPDCVVKVAEFLAEHPNVTLTISAARLYYYWETD | |
| | Y | |
| Macaca mulatta | RNTVWLCYEVKTRGPSMPTWDTKIFRGQVYSKP | SEQ ID NO: 106 |
| A3F (AA30- | EHHAEMCFLSRFCGNQLPAYKRFQITWFVSWTPC | |
| AA137) | PDCVAKVAEFLAEHPNVTLTISAARLYYYWETDY | |
| | RRALCRL | |
| Theropithecus | RNTVWLCYEVKTRGPSMPTWGTKIFRGQVYFQP | SEQ ID NO: 107 |
| gelada A3F | QYHAEMCFLSRFCGNQLPAYKRFQITWFVSWNPC | |
| (AA30-AA137) | PDCVAKVIEFLAEHPNVTLTISAARLYYYWGRDW | |
| | RRALRRL | |
| Cercocebus atys | GRSYTWLCYEVKIRKDPSKLPWYTGVFRGQVYS | SEQ ID NO: 108 |
| A3B (AA29- | KPEHHAEMCFLSRFCGNQLPAYKRFQITWFVSWN | |
| AA138) | PCPDCVAKVIEFLAEHPNVTLTISAARLYYYWSRD | |
| | WQRALCRL | |
| Macaca | GRSYTWLCYEVKIRKDPSKLPWDTGVFRGQVYS | SEQ ID NO: 109 |
| fascicularis A3B | KPEHHAEMCFLSRFCGNQLPAYKRFQITWFVSWN | |
| (AA29-AA138) | PCPDCVAKVIEFLAEHPNVTLTISTARLYYYWGR | |
| | DWQRALCRL | |
| Macaca mulatta | GRSYTWLCYEVKIRKDPSKLPWDTGVFRGQVYS | SEQ ID NO: 110 |
| A3B (AA29- | KPEHHAEMCFLSRFCGNQLPAYKRFQITWFVSWN | |
| AA138) | PCPDCVAKVIEFLAEHPNVTLTISTARLYYYWGR | |
| | DWQRALCRL | |
| Macaca leonina | GRSYTWLCYEVKIRKDPSKLPWDTGVFRGQVYS | SEQ ID NO: 111 |
| A3B (AA29- | KPEHHAEMCFLSRFCGNQLPAYKRFQITWFVSWN | |
| AA138) | PCPDCVVKVIEFLAEHPNVTLTISTARLYYYWGR | |
| | DWQRALCRL | |
| Mandrillus | GRSYTWLCYEVKIRKDPSKLPWYTGVFRGQVYS | SEQ ID NO: 112 |
| leucophaeus A3B | KPEHHAEMCFLSRFCGNQLPAYKRFQITWFVSWN | |
| (AA29-AA138) | PCPDCVAKVIEFLAEHPNVTLTIFTARLYYYWGR | |
| | DWQRALCRL | |
| Macaca | GRSYTWLCYEVKIRKDPSKLPWDTGVFRGQVYS | SEQ ID NO: 113 |
| nemestrina A3B | KPEHHAEMCFLSRFCGNQLPAYKRFQITWFVSWN | |
| (AA29-AA138) | PCPDCVAKVTEFLAEHPNVTLTISTARLYYYWGR | |
| | DWQRALCRL | |
| Rhinopithecus | GRSYTWLCYEVKIRKDPSKLPWDTGVFRGQVYSE | SEQ ID NO: 114 |
| bieti A3F (AA29- | PEHHAEMYFLSWFCGNQLPAYKRFQITWFVSWTP | |
| AA138) | CPDCVAKVAEFLTEHPNVTLTISAARLYYYRGRD | |
| | WRRALCRL | |
| Rhinopithecus | GRSYTWLCYEVKIRKDPSKLPWDTGVFRGQVYSE | SEQ ID NO: 115 |
| roxellana A3B | PEHHAEMYFLSWFCGNQLPAYKRFQITWFVSWTP | |
| (AA29-AA138) | CPDCVAKVAEFLTEHPNVTLTISAARLYYYRGRD | |
| | WRRALCRL | |
| Chlorocebus | GRSYTWLCYEVKIRKDPSKLPWDTGVFRGQMYS | SEQ ID NO: 116 |
| sabaeus A3B | KPEHHAEMCFLSWFCGNQLPAHKRFQITWFVSW | |
| (AA29-AA138) | TPCPDCVAKVAEFLAEYPNVTLTISAARLYYYWE | |
| | TDYRRALCRL | |
| Nomascus | RSYTWLCYEVKIRKDPSKLPWDTGVFRGQMYFQ | SEQ ID NO: 117 |
| leucogenys A3B | PEYHAEMCFLSWFCGNQLPAYKRFQITWFVSWTP | |
| (AA30-AA138) | CPDCVAKVAVFLAEHPNVTLTISAARLYYYWEK | |
| | DWQRALCRL | |
| Cercocebus atys | GRSYTWLCYEVKIKKYPSKLLWDTGVFQGQVYF | SEQ ID NO: 118 |
| A3F (AA29- | QPQYHAEMCFLSRFCGNQLPAYKRFQITWFVSW | |
| AA138) | NPCPDCVAKVTEFLAEHPNVTLTISAARLYYYWE | |
| | KDXRRALRRL | |
| Papio anubis A3F | GRSYTWLCYEVKIKEDPSKLLWDTGVFQGQVYF | SEQ ID NO: 119 |
| (AA29-AA138) | QPQYHAEMCFLSRFCGNQLPAYKRFQITWFVSW | |
| | NPCPDCVAKVTEFLAEHPNVTLTISAARLYYYWG | |
| | RDWRRALRRL | |
| Chlorocebus | GRRYTWLCYEVKIKKDPSKLPWDTGVFPGQVRP | SEQ ID NO: 120 |
| aethiops A3D | KFQSNRRYEVYFQPQYHAEMYFLSWFCGNQLPA | |
| (AA29-AA150) | YKHFQITWFVSWNPCPDCVAKVTEFLAEHRNVTL | |
| | TISAARLYYYWGKDWRRALCRL | |
| Chlorocebus | GRRYTWLCYEVKIKKDPSKLPWDTGVFPGQPQY | SEQ ID NO: 121 |
| sabaeus A3D | HAEMYFLSWFCGNQLPAYKHFQITWFVSWNPCP | |
| (AA29-AA134) | DCVAKVTEFLAEHRNVTLTISAARLYYYWGKDW | |
| | RRALCRL | |
| Chlorocebus | GRRYTWLCYEVKIKKDPSKLPWDTGVFPGQVRP | SEQ ID NO: 122 |
| sabaeus A3F | KFQSNRRQKVYFQPQYHAEMYFLSWFCGNQLPA | |
| (AA29-AA150) | YKHFQITWFVSWNPCPDCVAKVTEFLAEHRNVTL | |
| | TISAARLYYYWGKDWRRALCRL | |
| Erythrocebus | GRRYTWLCYEVKIKKDPSKLPWDTGVFQGQVRP | SEQ ID NO: 123 |
| patas A3D | KFQSNRRYEVYFQPQYHAEMCFLSWFCGNQLPA | |
| (AA29-AA150) | YKHFQITWFVSWNPCPDCVAKVTEFLAEHPNVTL | |
| | TISAARLYYYWGKDWRRALCRL | |
| Macaca | GRSYTWLCYEVKIRKDPSKLPWDTGVFRGQVRP | SEQ ID NO: 124 |
| fascicularis A3D | KLQSNRRYELSNWECRKRVYFQPQYHAEMYFLS | |
| (AA29-AA159) | WFCGNQLPANKRFQITWFASWNPCPDCVAKVTE | |
| | FLAEHPNVTLTISVARLYYYRGKDWRRALRRL | |
| Macaca | GRSYTWLCYEVKIRKDPSKLPWDTGVFRGQVYF | SEQ ID NO: 125 |
| fascicularis A3F | QPQYHAEMYFLSWFCGNQLPANKRFQITWFASW | |
| (AA29-AA138) | NPCPDCVAKVTEFLAEHPNVTLTISVARLYYYRG | |
| | KDWRRALRRL | |
| Macaca | GRSYTWLCYEVKIRKDPSKLPWDTGVFRDQVYF | SEQ ID NO: 126 |
| nemestrina A3D | QPQYHAEMCFLSWFCGNQLPANKRFQITWFVSW | |
| (AA29-AA138) | NPCPDCVTKVTEFLAEHPNVTLTISVARLYYYRG | |
| | KDWRRALRRL | |
| Macaca leonina | GRSYTWLCYEVKIRKDPSKLPWYTGVFRGQVYF | SEQ ID NO: 127 |
| A3D (AA29- | QPQYHAEMCFLSWFCGNQLPANKRFQITWFVSW | |
| AA138) | NPCPDCVAKVTEFLAEHPNVTLTISVARLYYYRG | |
| | KDWRRALRRL | |
| Macaca mulatta | GRSYTWLCYEVKIRKDPSKLPWDTGVFRGQVYF | SEQ ID NO: 128 |
| A3D (AA29- | QPQYHAEMCFLSWFCGNQLPAYKRFQITWFVSW | |
| AA138) | NPCPDCVAKVTEFLAEHPNVTLTISVARLYYYRG | |
| | KDWRRALCRL | |
| Gorilla A3D | GRSYTWLCYEVKIRRGSSNLLWNTGVFRGPVPPK | SEQ ID NO: 129 |
| (AA29-AA150) | LQSNHRQEVYFQFENHAEMCFLSWFCGNRLPAN | |
| | RRFQITWFVSWNPCLPCVVKVTKFLAEHPNVTLTI | |
| | SAARLYYYRDREWRRVLRRL | |
| Pan paniscus | GRSYTWLCYEVKIKRGCSNLIWDTGVFRGPVLPK | SEQ ID NO: 130 |
| A3D (AA29- | LQSNHRQEVYFQFENHAEMCFFSWFCGNRLPAN | |
| AA150) | RRFQITWFVSWNPCLPCVVKVTKFLAEHPNVTLTI | |
| | SAARLYYYQDREWRRVLRRL | |
| Pan troglodytes | GRSYTWLCYEVKIKRGCSNLIWDTGVFRGPVLPK | SEQ ID NO: 131 |
| A3D (AA29- | LQSNHRQEVYFQFENHAEMCFFSWFCGNRLPAN | |
| AA150) | RRFQITWFVSWNPCLPCVVKVTKFLAEHPNVTLTI | |
| | SAARLYYYQDREWRRVLRRL | |
| Homo sapiens | GRSYTWLCYEVKIKRGRSNLLWDTGVFRGPVLPK | SEQ ID NO: 132 |
| A3D (AA29- | RQSNHRQEVYFRFENHAEMCFLSWFCGNRLPAN | |
| AA150) | RRFQITWFVSWNPCLPCVVKVTKFLAEHPNVTLTI | |
| | SAARLYYYRDRDWRWVLLRL | |
| Nomascus | GRSYTWLCYEVKIRKDPSKLPWDKGVFRGQVLP | SEQ ID NO: 133 |
| leucogenys A3D | KFQSNHRQEVYFQLENHAEMCFLSWFCGNQLPA | |
| (AA29-AA150) | NRRFQITWFVSWNPCLPCVAKVTEFLAEHPNVTL | |
| | TISAARLYYYRGRDWRRALRRL | |
| Saimiri | GKKYTWLCYEVKIKKDTSKLPWNTGVFRGQVNF | SEQ ID NO: 134 |
| boliviensis A3C | NPEHHAEMYFLSWFRGKLLPACKRSQITWFVSW | |
| (AA29-AA138) | NPCLYCVAKVAEFLAEHPNVTLTVSTARLYCYW | |
| | KKDWRRALRKL | |
| Saimiri | GKKYTWLCYEVKIKKDTSKLPWNTGVFRGQVNF | SEQ ID NO: 135 |
| boliviensis A3F | NPEHHAEMYFLSWFRGKLLPACKRSQITWFVSW | |
| (AA29-AA138) | NPCLYCVAKVAEFLAEHPNVTLTVSTARLYCYW | |
| | KKDWRRALRKL | |
| Piliocolobus | GRRYTWLCYEVKIMKDHSKLPWYTGVFRGQVYF | SEQ ID NO: 136 |
| tephrosceles A3F | EPQNHAEMCFLSWFCGNQLPAYECCQITWFVSW | |
| (AA36-AA145) | TPCPDCVAKVTEFLAEHPNVTLTISAARLYYYRG | |
| | RDWRRALRRL | |
| Colobus | GRRYTWLCYEVKISKDPSKLPWDTGIFRGQVYFE | SEQ ID NO: 137 |
| angolensis | PQYHAEMCFLSWYCGNQLPAYKCFQITWFVSWT | |
| palliatus A3F | PCPDCVGKVAEFLAEHPNVTLTISAARLYYYWET | |
| (AA29-AA138) | DYRRALCRL | |
| Pongo abelii A3F | RNYTWLCYEVKIRKDPSKLAWDTGVFRGQVLPK | SEQ ID NO: 138 |
| (AA30-AA150) | LQSNHRREVYFEPQYHAEMCFLSWFCGNQLSAY | |
| | ERFQITWFVSWTPCPDCVAMLAEFLAEHPNVTLT | |
| | VSAARLYYYWERDYRGALRRL | |

The term “nucleobase deamiinase” as used herein, refers to a group of enzymes that catalyze the hydrolytic deamiination of nucleobases such as cytidine, deoxycytidine, adenosine and deoxyadenosine. Non-limiting examples of nucleobase deaminases include cytidine deaminases and adenosine deaminases.

Some of the nucleobase deamiinases have a single, catalytic domain, while others also have other domains, such as an inhibitory domain as described in WO2020156575A1. In some embodiments, therefore, the gene editing system disclosed herein only includes the catalytic domain, such as mouse A3 cytidine deaminase domain 1 (mA3-CDA1, SEQ ID NO: 44) and human A3B cytidine deaminase domain 2 (hA3B-CDA2, SEQ ID NO: 45). In some embodiments, the gene editing system disclosed herein includes at least a catalytic core of the catalytic domain. For instance, when mA3-CDA1 was truncated at residues 1961197 the CDA1 domain still retained substantial editing efficiencies.

TABLE 5

| | | |
|---|---|---|
| mouse | MSSSTLSNICLTKGLPETRFWVEG | SEQ ID |
| APOBEC3 | RRMDPLSEEEFYSQFYNQRVKHLC | NO: 42 |
| cytidine | YYHRMKPYLCYQLEQFNGQAPLKG | |
| deaminase | CLLSEKGKQHAEILFLDKIRSMEL | |
| domain 2 | SQVTITCYLTWSPCPNCAWQLAAF | |
| (mA3-CDA2) | KRDRPDLILHIYTSRLYFHWKRPF | |
| | QKGLCSLWQSGILVDVMDLPQFTD | |
| | CWTNFVNPKRPFWPWKGLEIISRR | |
| | TQRRLRRIKESWGLQDLVNDFGNL | |
| | QLGPPMS | |
| human | MNPQIRNPMERMYRDTFYDNFENE | SEQ ID |
| APOBEC3B | PILYGRSYTWLCYEVKIKRGRSNL | NO: 43 |
| cytidine | LWDTGVFRGQVYFKPQYHAEMCFL | |
| deaminase | SWFCGNQLPAYKCFQITWFVSWTP | |
| domain 1 | CPDCVAKLAEFLSEHPNVTLTISA | |
| (hA3B-CDA1) | ARLYYYWERDYRRALCRLSQAGAR | |
| | VKIMDYEEFAYCWENFVYNEGQ | |
| mouse | MGPFCLGCSHRKCYSPIRNLISQE | SEQ ID |
| APOBEC3 | TFKFHFKNLGYAKGRKDTFLCYEV | NO: 44 |
| cytidine | TRKDCDSPVSLHHGVFKNKDNIHA | |
| deaminase | EICFLYWFHDKVLKVLSPREEFKI | |
| domain 1 | TWYMSWSPCFECAEQIVRFLATHH | |
| (mA3-CDA1) | NLSLDIFSSRLYNVQDPETQQNLC | |
| | RLVQEGAQVAAMDLYEFKKCWKKF | |
| | VDNGGRRFRPWKRLLTNFRYQDSK | |
| | LQEILRPCYISVPSS | |
| human | MQFMPWYKFDENYAFLHRTLKEIL | SEQ ID |
| APOBEC3B | RYLMDPDTFTFNFNNDPLVLRRRQ | NO: 45 |
| cytidine | TYLCYEVERLDNGTWVLMDQHMGF | |
| deaminase | LCNEAKNLLCGFYGRHAELRFLDL | |
| domain 2 | VPSLQLDPAQIYRVTWFISWSPCF | |
| (hA3B-CDA2) | SWGCAGEVRAFLQENTHVRLRIFA | |
| | ARIYDYDPLYKEALQMLRDAGAQV | |
| | SIMTYDEFEYCWDTFVYRQGCPFQ | |
| | PWDGLEEHSQALSGRLRAILQNQG | |
| | N | |

“Adenosine deaminase” refers to an enzyme of the purine metabolism which catalyzes the irreversible deamination of adenosine and deoxyadenosine to inosine and deoxyinosine, respectively.

In some embodiments of the gene editing system described herein, the nucleotide deaminase is an adenosine deaminase.

In some embodiments of the gene editing system described herein, the adenosine deaminase is selected from the group consisting of tRNA-specific adenosine deaminase (TadA), adenosine deaminase tRNA specific 1 (ADAT1), adenosine deaminase tRNA specific 2 (ADAT2), adenosine deaminase tRNA specific 3 (ADAT3), adenosine deaminase RNA specific B1 (ADARB1), adenosine deaminase RNA specific B2 (ADARB2), adenosine monophosphate deaminase 1 (AMPD1), adenosine monophosphate deaminase 2 (AMPD2), adenosine monophosphate deaminase 3 (AMPD3), adenosine deaminase (ADA), adenosine deaminase 2 (ADA2), adenosine deaminase like (ADAL), adenosine deaminase domain containing 1 (ADAD1), adenosine deaminase domain containing 2 (ADAD2), and adenosine deaminase RNA specific (ADAR).

In some embodiments of the gene editing system described herein, the adenosine deaminase comprises an amino acid sequence of SEQ ID NO: 159-251.

In some embodiments of the gene editing system described herein, the adenosine deaminase is a naturally occurring adenosine deaminase, an engineered adenosine deaminase, an evolved adenosine deaminase, or a cytidine deaminase that possesses adenosine deaminase activity.

In some embodiments of the gene editing system described herein, the adenosine deaminase is a human or mouse adenosine deaminase.

In some embodiments of the gene editing system described herein, the first fusion protein comprises one or more nucleotide deaminase, and the one or more nucleotide deaminase are the same or different.

In some embodiments of the gene editing system described herein, each of the one or more nucleotide deaminase is a cytidine deaminase or an adenosine deaminase.

In some embodiments of the gene editing system described herein, the nucleotide deaminase is a fusion of at least one cytidine deaminase and at least one adenosine deaminase.

In some embodiments of the gene editing system described herein, the first fusion protein further comprises one or more copies of uracil glycosylase inhibitor (UGI).

The “Uracil Glycosylase Inhibitor” (UGI), which can be prepared from Bacillus subtilis bacteriophage PBS1, is a small protein (9.5 kDa) which inhibits E. coli uracil-DNA glycosylase (UDG) as well as UDG from other species. Inhibition of UDG occurs by reversible protein binding with a 1:1 UDG:UGI stoichiometry. UGI is capable of dissociating UDG-DNA complexes. A non-limiting example of UGI is found in Bacillus phage AR9 (YP_009283008.1). In some embodiments, the UGI comprises the amino acid sequence of SEQ ID NO: 46 or has at least 70%, 75%, 80%, 85%, 90% or 95% sequence identity to SEQ ID NO: 46 and retains the uracil glycosylase inhibition activity.

In some embodiments, the first fusion protein further comprises a nuclear localization sequence (NLS).

A “nuclear localization signal or sequence” (NLS) is an amino acid sequence that tags a protein for import into the cell nucleus by nuclear transport. Typically, this signal consists of one or more short sequences of positively charged lysines or arginines exposed on the protein surface. Different nuclear localized proteins may share the same NLS. A non-limiting example of NLS is the internal SV40 nuclear localization sequence (iNLS).

In some embodiments, a peptide linker is optionally provided between each of the fragments in any of the fusion proteins. In some embodiments, the peptide linker has from 1 to 100 amino acid residues (or 3-20, 4-15, without limitation). In some embodiments, at least 10%, 20%, 30%, 40%, 50%, 60%, 70%, 80% or 90% of the amino acid residues of peptide linker are amino acid residues selected from the group consisting of alanine, glycine, cysteine, and serine.

In some embodiments of the gene editing system described herein, each of the Cas protein is a Cas9, a dead Cas9 (dCas9), or a Cas9 nickase (nCas9) selected from the group consisting of SpCas9, FnCas9, St1Cas9, St3Cas9, NmCas9, SaCas9, AsCpf1, LbCpf1, FnCpf1, VQR Cas9, EQR Cas9, VRER Cas9, Cas9-NG, xCas9, eCas9, SpCas9-HF1, HypaCas9, HiFiCas9, sniper-Cas9, SpG, SpRY, KKH SaCas9, CjCas9, Cas9-NRRH, Cas9-NRCH, Cas9-NRTH, SsCpf1, PcCpf1, BpCpf1, LiCpf1, PmCpf1, Lb2Cpf1, PbCpf1, PbCpf1, PeCpf1, PdCpf1, MbCpf1, EeCpf1, CmtCpf1, BsCpf1, BhCasl2b, AkCasl2b, BsCasl2b, AmCasl2b, AaCasl2b, RfxCasl3d, LwaCasl3a, PspCasl3b, PguCasl3b, and RanCasl3b.

The term “Cas protein” or “clustered regularly interspaced short palindromic repeats (CRISPR)-associated (Cas) protein” refers to RNA-guided DNA endonuclease enzymes associated with the CRISPR (Clustered Regularly Interspaced Short Palindromic Repeats) adaptive immunity system in Streptococcus pyogenes, as well as other bacteria. Cas proteins include Cas9 proteins, Cas12a (Cpf1) proteins, Cas12b (formerly known as C2cl) proteins, Cas13 proteins and various engineered counterparts. Example Cas proteins include SpCas9, FnCas9, St1Cas9, St3Cas9, NmCas9, SaCas9, AsCpf1, LbCpf1, FnCpf1, VQR Cas9, EQR Cas9, VRER Cas9, Cas9-NG, xCas9, eCas9, SpCas9-HF1, HypaCas9, HiFiCas9, sniper-Cas9, SpG, SpRY, KKH SaCas9, CjCas9, Cas9-NRRH, Cas9-NRCH, Cas9-NRTH, SsCpf1, PcCpf1, BpCpf1, LiCpf1, PmCpf1, Lb2Cpf1, PbCpf1, PbCpf1, PeCpf1, PdCpf1, MbCpf1, EeCpf1, CmtCpf1, BsCpf1, BhCasl2b, AkCasl2b, BsCasl2b, AmCasl2b, AaCasl2b, RfxCasl3d, LwaCasl3a, PspCasl3b, PguCasl3b and RanCasl3b.

In some embodiments, the Cas protein comprises an amino acid sequence selected from the table 6 below or SEQ ID NOs: 308-359.

TABLE 6
Exemplary Cas Proteins

| Cas protein types | Cas proteins |
|---|---|
| Cas9 proteins | Cas9 from Staphylococcus aureus (SaCas9) |
| | Cas9 from Neisseria meningitidis (NmeCas9) |
| | Cas9 from Streptococcus thermophilus (StCas9) |
| | Cas9 from Campylobacter jejuni (CjCas9) |
| Cas12a (Cpf1) | Cas12a (Cpf1) from Acidaminococcus sp BV3L6 (AsCpf1) |
| proteins | Cas12a (Cpf1) from Francisella novicida sp BV3L6 (FnCpf1) |
| | Cas12a (Cpf1) from Smithella sp SC K08D17 (SsCpf1) |
| | Cas12a (Cpf1) from Porphyromonas crevioricanis (PcCpf1) |
| | Cas12a (Cpf1) from Butyrivibrio proteoclasticus (BpCpf1) |
| | Cas12a (Cpf1) from Candidatus Methanoplasma termitum (CmtCpf1) |
| | Cas12a (Cpf1) from Leptospira inadai (LiCpf1) |
| | Cas12a (Cpf1) from Porphyromonas macacae (PmCpf1) |
| | Cas12a (Cpf1) from Peregrinibacteria bacterium GW2011 WA2 33 10 (Pb3310Cpf1) |
| | Cas12a (Cpf1) from Parcubacteria bacterium GW2011 GWC2 44 17 (Pb4417Cpf1) |
| | Cas12a (Cpf1) from Butyrivibrio sp. NC3005 (BsCpf1) |
| | Cas12a (Cpf1) from Eubacterium eligens (EeCpf1) |
| Cas12b (C2c1) | Cas12b (C2c1) Bacillus hisashii (BhCas12b) |
| proteins | Cas12b (C2c1) Bacillus hisashii with a gain-of-function mutation |
| | (see, e.g., Strecker et al., Nature Communications 10 (article 212) (2019) |
| | Cas12b (C2c1) Alicyclobacillus kakegawensis (AkCas12b) |
| | Cas12b (C2c1) Elusimicrobia bacterium (EbCas12b) |
| | Cas12b (C2c1) Laceyella sediminis (Ls) (LsCas12b) |
| Cas13 proteins | Cas13d from Ruminococcus flavefaciens XPD3002 (RfCas13d) |
| | Cas13a from Leptotrichia wadei (LwaCas13a) |
| | Cas13b from Prevotella sp. P5-125 (PspCas13b) |
| | Cas13b from Porphyromonas gulae (PguCas13b) |
| | Cas13b from Riemerella anatipestifer (RanCas13b) |
| Engineered Cas | Nickases (mutation in one nuclease domain) |
| proteins | Catalytically inactive mutant (dCas9; mutations in both of the nuclease domains) |
| | Enhanced variants with improved specificity (see, e.g., Chen et |
| | al., Nature, 550, 407-410 (2017) |

In some embodiments, the Cas protein is a Cas9, a dead Cas9 (dCas9), or a Cas9 nickase (nCas9).

In some embodiments, the Cas protein is a nCas9. In some embodiments, the nCas9 protein is a nCas9-D10A protein. In some embodiments, the nCas9-D10A protein has an amino acid sequence of SEQ ID NO: 47. In some embodiments, the Cas protein comprises an amino acid sequence of any one of SEQ ID NOs: 308-359. (Table 11)

TABLE 11
Cas proteins

| Name | Sequence | SEQ ID |
|---|---|---|
| SpCas9 | MDKKYSIGLDIGTNSVGWAVITDEYKVPSKKFKVLGNTDRHS | SEQ ID |
| | IKKNLIGALLFDSGETAEATRLKRTARRRYTRRKNRICYLQEIF | NO. |
| | SNEMAKVDDSFFHRLEESFLVEEDKKHERHPIFGNIVDEVAYH | 308 |
| | EKYPTIYHLRKKLVDSTDKADLRLIYLALAHMIKFRGHFLIEG | |
| | DLNPDNSDVDKLFIQLVQTYNQLFEENPINASGVDAKAILSAR | |
| | LSKSRRLENLIAQLPGEKKNGLFGNLIALSLGLTPNFKSNFDLA | |
| | EDAKLQLSKDTYDDDLDNLLAQIGDQYADLFLAAKNLSDAIL | |
| | LSDILRVNTEITKAPLSASMIKRYDEHHQDLTLLKALVRQQLP | |
| | EKYKEIFFDQSKNGYAGYIDGGASQEEFYKFIKPILEKMDGTE | |
| | ELLVKLNREDLLRKQRTFDNGSIPHQIHLGELHAILRRQEDFY | |
| | PFLKDNREKIEKILTFRIPYYVGPLARGNSRFAWMTRKSEETIT | |
| | PWNFEEVVDKGASAQSFIERMTNFDKNLPNEKVLPKHSLLYE | |
| | YFTVYNELTKVKYVTEGMRKPAFLSGEQKKAIVDLLFKTNRK | |
| | VTVKQLKEDYFKKIECFDSVEISGVEDRFNASLGTYHDLLKIIK | |
| | DKDFLDNEENEDILEDIVLTLTLFEDREMIEERLKTYAHLFDD | |
| | KVMKQLKRRRYTGWGRLSRKLINGIRDKQSGKTILDFLKSDG | |
| | FANRNFMQLIHDDSLTFKEDIQKAQVSGQGDSLHEHIANLAG | |
| | SPAIKKGILQTVKVVDELVKVMGRHKPENIVIEMARENQTTQ | |
| | KGQKNSRERMKRIEEGIKELGSQILKEHPVENTQLQNEKLYLY | |
| | YLQNGRDMYVDQELDINRLSDYDVDHIVPQSFLKDDSIDNKV | |
| | LTRSDKNRGKSDNVPSEEVVKKMKNYWRQLLNAKLITQRKF | |
| | DNLTKAERGGLSELDKAGFIKRQLVETRQITKHVAQILDSRM | |
| | NTKYDENDKLIREVKVITLKSKLVSDFRKDFQFYKVREINNYH | |
| | HAHDAYLNAVVGTALIKKYPKLESEFVYGDYKVYDVRKMIA | |
| | KSEQEIGKATAKYFFYSNIMNFFKTEITLANGEIRKRPLIETNG | |
| | ETGEIVWDKGRDFATVRKVLSMPQVNIVKKTEVQTGGFSKES | |
| | ILPKRNSDKLIARKKDWDPKKYGGFDSPTVAYSVLVVAKVEK | |
| | GKSKKLKSVKELLGITIMERSSFEKNPIDFLEAKGYKEVKKDLI | |
| | IKLPKYSLFELENGRKRMLASAGELQKGNELALPSKYVNFLY | |
| | LASHYEKLKGSPEDNEQKQLFVEQHKHYLDEIIEQISEFSKRVI | |
| | LADANLDKVLSAYNKHRDKPIREQAENIIHLFTLTNLGAPAAF | |
| | KYFDTTIDRKRYTSTKEVLDATLIHQSITGLYETRIDLSQLGGD | |
| dSpCas9 | MDKKYSIGLAIGTNSVGWAVITDEYKVPSKKFKVLGNTDRHS | SEQ ID |
| | IKKNLIGALLFDSGETAEATRLKRTARRRYTRRKNRICYLQEIF | NO. |
| | SNEMAKVDDSFFHRLEESFLVEEDKKHERHPIFGNIVDEVAYH | 309 |
| | EKYPTIYHLRKKLVDSTDKADLRLIYLALAHMIKFRGHFLIEG | |
| | DLNPDNSDVDKLFIQLVQTYNQLFEENPINASGVDAKAILSAR | |
| | LSKSRRLENLIAQLPGEKKNGLFGNLIALSLGLTPNFKSNFDLA | |
| | EDAKLQLSKDTYDDDLDNLLAQIGDQYADLFLAAKNLSDAIL | |
| | LSDILRVNTEITKAPLSASMIKRYDEHHQDLTLLKALVRQQLP | |
| | EKYKEIFFDQSKNGYAGYIDGGASQEEFYKFIKPILEKMDGTE | |
| | ELLVKLNREDLLRKQRTFDNGSIPHQIHLGELHAILRRQEDFY | |
| | PFLKDNREKIEKILTFRIPYYVGPLARGNSRFAWMTRKSEETIT | |
| | PWNFEEVVDKGASAQSFIERMTNFDKNLPNEKVLPKHSLLYE | |
| | YFTVYNELTKVKYVTEGMRKPAFLSGEQKKAIVDLLFKTNRK | |
| | VTVKQLKEDYFKKIECFDSVEISGVEDRFNASLGTYHDLLKIIK | |
| | DKDFLDNEENEDILEDIVLTLTLFEDREMIEERLKTYAHLFDD | |
| | KVMKQLKRRRYTGWGRLSRKLINGIRDKQSGKTILDFLKSDG | |
| | FANRNFMQLIHDDSLTFKEDIQKAQVSGQGDSLHEHIANLAG | |
| | SPAIKKGILQTVKVVDELVKVMGRHKPENIVIEMARENQTTQ | |
| | KGQKNSRERMKRIEEGIKELGSQILKEHPVENTQLQNEKLYLY | |
| | YLQNGRDMYVDQELDINRLSDYDVDAIVPQSFLKDDSIDNKV | |
| | LTRSDKNRGKSDNVPSEEVVKKMKNYWRQLLNAKLITQRKF | |
| | DNLTKAERGGLSELDKAGFIKRQLVETRQITKHVAQILDSRM | |
| | NTKYDENDKLIREVKVITLKSKLVSDFRKDFQFYKVREINNYH | |
| | HAHDAYLNAVVGTALIKKYPKLESEFVYGDYKVYDVRKMIA | |
| | KSEQEIGKATAKYFFYSNIMNFFKTEITLANGEIRKRPLIETNG | |
| | ETGEIVWDKGRDFATVRKVLSMPQVNIVKKTEVQTGGFSKES | |
| | ILPKRNSDKLIARKKDWDPKKYGGFDSPTVAYSVLVVAKVEK | |
| | GKSKKLKSVKELLGITIMERSSFEKNPIDFLEAKGYKEVKKDLI | |
| | IKLPKYSLFELENGRKRMLASAGELQKGNELALPSKYVNFLY | |
| | LASHYEKLKGSPEDNEQKQLFVEQHKHYLDEIIEQISEFSKRVI | |
| | LADANLDKVLSAYNKHRDKPIREQAENIIHLFTLTNLGAPAAF | |
| | KYFDTTIDRKRYTSTKEVLDATLIHQSITGLYETRIDLSQLGGD | |
| nSpCas9 | MDKKYSIGLAIGTNSVGWAVITDEYKVPSKKFKVLGNTDRHS | SEQ ID |
| (D10A) | IKKNLIGALLFDSGETAEATRLKRTARRRYTRRKNRICYLQEIF | NO. |
| | SNEMAKVDDSFFHRLEESFLVEEDKKHERHPIFGNIVDEVAYH | 310 |
| | EKYPTIYHLRKKLVDSTDKADLRLIYLALAHMIKFRGHFLIEG | |
| | DLNPDNSDVDKLFIQLVQTYNQLFEENPINASGVDAKAILSAR | |
| | LSKSRRLENLIAQLPGEKKNGLFGNLIALSLGLTPNFKSNFDLA | |
| | EDAKLQLSKDTYDDDLDNLLAQIGDQYADLFLAAKNLSDAIL | |
| | LSDILRVNTEITKAPLSASMIKRYDEHHQDLTLLKALVRQQLP | |
| | EKYKEIFFDQSKNGYAGYIDGGASQEEFYKFIKPILEKMDGTE | |
| | ELLVKLNREDLLRKQRTFDNGSIPHQIHLGELHAILRRQEDFY | |
| | PFLKDNREKIEKILTFRIPYYVGPLARGNSRFAWMTRKSEETIT | |
| | PWNFEEVVDKGASAQSFIERMTNFDKNLPNEKVLPKHSLLYE | |
| | YFTVYNELTKVKYVTEGMRKPAFLSGEQKKAIVDLLFKTNRK | |
| | VTVKQLKEDYFKKIECFDSVEISGVEDRFNASLGTYHDLLKIIK | |
| | DKDFLDNEENEDILEDIVLTLTLFEDREMIEERLKTYAHLFDD | |
| | KVMKQLKRRRYTGWGRLSRKLINGIRDKQSGKTILDFLKSDG | |
| | FANRNFMQLIHDDSLTFKEDIQKAQVSGQGDSLHEHIANLAG | |
| | SPAIKKGILQTVKVVDELVKVMGRHKPENIVIEMARENQTTQ | |
| | KGQKNSRERMKRIEEGIKELGSQILKEHPVENTQLQNEKLYLY | |
| | YLQNGRDMYVDQELDINRLSDYDVDHIVPQSFLKDDSIDNKV | |
| | LTRSDKNRGKSDNVPSEEVVKKMKNYWRQLLNAKLITQRKF | |
| | DNLTKAERGGLSELDKAGFIKRQLVETRQITKHVAQILDSRM | |
| | NTKYDENDKLIREVKVITLKSKLVSDFRKDFQFYKVREINNYH | |
| | HAHDAYLNAVVGTALIKKYPKLESEFVYGDYKVYDVRKMIA | |
| | KSEQEIGKATAKYFFYSNIMNFFKTEITLANGEIRKRPLIETNG | |
| | ETGEIVWDKGRDFATVRKVLSMPQVNIVKKTEVQTGGFSKES | |
| | ILPKRNSDKLIARKKDWDPKKYGGFDSPTVAYSVLVVAKVEK | |
| | GKSKKLKSVKELLGITIMERSSFEKNPIDFLEAKGYKEVKKDLI | |
| | IKLPKYSLFELENGRKRMLASAGELQKGNELALPSKYVNFLY | |
| | LASHYEKLKGSPEDNEQKQLFVEQHKHYLDEIIEQISEFSKRVI | |
| | LADANLDKVLSAYNKHRDKPIREQAENIIHLFTLTNLGAPAAF | |
| | KYFDTTIDRKRYTSTKEVLDATLIHQSITGLYETRIDLSQLGGD | |
| nSpCas9 | MDKKYSIGLDIGTNSVGWAVITDEYKVPSKKFKVLGNTDRHS | SEQ ID |
| (H840A) | IKKNLIGALLFDSGETAEATRLKRTARRRYTRRKNRICYLQEIF | NO. |
| | SNEMAKVDDSFFHRLEESFLVEEDKKHERHPIFGNIVDEVAYH | 311 |
| | EKYPTIYHLRKKLVDSTDKADLRLIYLALAHMIKFRGHFLIEG | |
| | DLNPDNSDVDKLFIQLVQTYNQLFEENPINASGVDAKAILSAR | |
| | LSKSRRLENLIAQLPGEKKNGLFGNLIALSLGLTPNFKSNFDLA | |
| | EDAKLQLSKDTYDDDLDNLLAQIGDQYADLFLAAKNLSDAIL | |
| | LSDILRVNTEITKAPLSASMIKRYDEHHQDLTLLKALVRQQLP | |
| | EKYKEIFFDQSKNGYAGYIDGGASQEEFYKFIKPILEKMDGTE | |
| | ELLVKLNREDLLRKQRTFDNGSIPHQIHLGELHAILRRQEDFY | |
| | PFLKDNREKIEKILTFRIPYYVGPLARGNSRFAWMTRKSEETIT | |
| | PWNFEEVVDKGASAQSFIERMTNFDKNLPNEKVLPKHSLLYE | |
| | YFTVYNELTKVKYVTEGMRKPAFLSGEQKKAIVDLLFKTNRK | |
| | VTVKQLKEDYFKKIECFDSVEISGVEDRFNASLGTYHDLLKIIK | |
| | DKDFLDNEENEDILEDIVLTLTLFEDREMIEERLKTYAHLFDD | |
| | KVMKQLKRRRYTGWGRLSRKLINGIRDKQSGKTILDFLKSDG | |
| | FANRNFMQLIHDDSLTFKEDIQKAQVSGQGDSLHEHIANLAG | |
| | SPAIKKGILQTVKVVDELVKVMGRHKPENIVIEMARENQTTQ | |
| | KGQKNSRERMKRIEEGIKELGSQILKEHPVENTQLQNEKLYLY | |
| | YLQNGRDMYVDQELDINRLSDYDVDAIVPQSFLKDDSIDNKV | |
| | LTRSDKNRGKSDNVPSEEVVKKMKNYWRQLLNAKLITQRKF | |
| | DNLTKAERGGLSELDKAGFIKRQLVETRQITKHVAQILDSRM | |
| | NTKYDENDKLIREVKVITLKSKLVSDFRKDFQFYKVREINNYH | |
| | HAHDAYLNAVVGTALIKKYPKLESEFVYGDYKVYDVRKMIA | |
| | KSEQEIGKATAKYFFYSNIMNFFKTEITLANGEIRKRPLIETNG | |
| | ETGEIVWDKGRDFATVRKVLSMPQVNIVKKTEVQTGGFSKES | |
| | ILPKRNSDKLIARKKDWDPKKYGGFDSPTVAYSVLVVAKVEK | |
| | GKSKKLKSVKELLGITIMERSSFEKNPIDFLEAKGYKEVKKDLI | |
| | IKLPKYSLFELENGRKRMLASAGELQKGNELALPSKYVNFLY | |
| | LASHYEKLKGSPEDNEQKQLFVEQHKHYLDEIIEQISEFSKRVI | |
| | LADANLDKVLSAYNKHRDKPIREQAENIIHLFTLTNLGAPAAF | |
| | KYFDTTIDRKRYTSTKEVLDATLIHQSITGLYETRIDLSQLGGD | |
| FnCas9 | MNFKILPIAIDLGVKNTGVFSAFYQKGTSLERLDNKNGKVYEL | SEQ ID |
| | SKDSYTLLMNNRTARRHQRRGIDRKQLVKRLFKLIWTEQLNL | NO. |
| | EWDKDTQQAISFLFNRRGFSFITDGYSPEYLNIVPEQVKAILM | 312 |
| | DIFDDYNGEDDLDSYLKLATEQESKISEIYNKLMQKILEFKLM | |
| | KLCTDIKDDKVSTKTLKEITSYEFELLADYLANYSESLKTQKF | |
| | SYTDKQGNLKELSYYHHDKYNIQEFLKRHATINDRILDTLLTD | |
| | DLDIWNFNFEKFDFDKNEEKLQNQEDKDHIQAHLHHFVFAVN | |
| | KIKSEMASGGRHRSQYFQEITNVLDENNHQEGYLKNFCENLH | |
| | NKKYSNLSVKNLVNLIGNLSNLELKPLRKYFNDKIHAKADHW | |
| | DEQKFTETYCHWILGEWRVGVKDQDKKDGAKYSYKDLCNE | |
| | LKQKVTKAGLVDFLLELDPCRTIPPYLDNNNRKPPKCQSLILN | |
| | PKFLDNQYPNWQQYLQELKKLQSIQNYLDSFETDLKVLKSSK | |
| | DQPYFVEYKSSNQQIASGQRDYKDLDARILQFIFDRVKASDEL | |
| | LLNEIYFQAKKLKQKASSELEKLESSKKLDEVIANSQLSQILKS | |
| | QHTNGIFEQGTFLHLVCKYYKQRQRARDSRLYIMPEYRYDKK | |
| | LHKYNNTGRFDDDNQLLTYCNHKPRQKRYQLLNDLAGVLQ | |
| | VSPNFLKDKIGSDDDLFISKWLVEHIRGFKKACEDSLKIQKDN | |
| | RGLLNHKINIARNTKGKCEKEIFNLICKIEGSEDKKGNYKHGL | |
| | AYELGVLLFGEPNEASKPEFDRKIKKFNSIYSFAQIQQIAFAER | |
| | KGNANTCAVCSADNAHRMQQIKITEPVEDNKDKIILSAKAQR | |
| | LPAIPTRIVDGAVKKMATILAKNIVDDNWQNIKQVLSAKHQL | |
| | HIPIITESNAFEFEPALADVKGKSLKDRRKKALERISPENIFKDK | |
| | NNRIKEFAKGISAYSGANLTDGDFDGAKEELDHIIPRSHKKYG | |
| | TLNDEANLICVTRGDNKNKGNRIFCLRDLADNYKLKQFETTD | |
| | DLEIEKKIADTIWDANKKDFKFGNYRSFINLTPQEQKAFRHAL | |
| | FLADENPIKQAVIRAINNRNRTFVNGTQRYFAEVLANNIYLRA | |
| | KKENLNTDKISFDYFGIPTIGNGRGIAEIRQLYEKVDSDIQAYA | |
| | KGDKPQASYSHLIDAMLAFCIAADEHRNDGSIGLEIDKNYSLY | |
| | PLDKNTGEVFTKDIFSQIKITDNEFSDKKLVRKKAIEGFNTHRQ | |
| | MTRDGIYAENYLPILIHKELNEVRKGYTWKNSEEIKIFKGKKY | |
| | DIQQLNNLVYCLKFVDKPISIDIQISTLEELRNILTTNNIAATAE | |
| | YYYINLKTQKLHEYYIENYNTALGYKKYSKEMEFLRSLAYRS | |
| | ERVKIKSIDDVKQVLDKDSNFIIGKITLPFKKEWQRLYREWQN | |
| | TTIKDDYEFLKSFFNVKSITKLHKKVRKDFSLPISTNEGKFLVK | |
| | RKTWDNNFIYQILNDSDSRADGTKPFIPAFDISKNEIVEAIIDSF | |
| | TSKNIFWLPKNIELQKVDNKNIFAIDTSKWFEVETPSDLRDIGI | |
| | ATIQYKIDNNSRPKVRVKLDYVIDDDSKINYFMNHSLLKSRYP | |
| | DKVLEILKQSTIIEFESSGFNKTIKEMLGMKLAGIYNETSNN | |
| St1Cas9 | MGSDLVLGLDIGIGSVGVGILNKVTGEIIHKNSRIFPAAQAENN | SEQ ID |
| | LVRRTNRQGRRLARRKKHRRVRLNRLFEESGLITDFTKISINL | NO. |
| | NPYQLRVKGLTDELSNEELFIALKNMVKHRGISYLDDASDDG | 313 |
| | NSSVGDYAQIVKENSKQLETKTPGQIQLERYQTYGQLRGDFT | |
| | VEKDGKKHRLINVFPTSAYRSEALRILQTQQEFNPQITDEFINR | |
| | YLEILTGKRKYYHGPGNEKSRTDYGRYRTSGETLDNIFGILIG | |
| | KCTFYPDEFRAAKASYTAQEFNLLNDLNNLTVPTETKKLSKE | |
| | QKNQIINYVKNEKAMGPAKLFKYIAKLLSCDVADIKGYRIDK | |
| | SGKAEIHTFEAYRKMKTLETLDIEQMDRETLDKLAYVLTLNT | |
| | EREGIQEALEHEFADGSFSQKQVDELVQFRKANSSIFGKGWH | |
| | NFSVKLMMELIPELYETSEEQMTILTRLGKQKTTSSSNKTKYI | |
| | DEKLLTEEIYNPVVAKSVRQAIKIVNAAIKEYGDFDNIVIEMA | |
| | RETNEDDEKKAIQKIQKANKDEKDAAMLKAANQYNGKAELP | |
| | HSVFHGHKQLATKIRLWHQQGERCLYTGKTISIHDLINNSNQF | |
| | EVDHILPLSITFDDSLANKVLVYATANQEKGQRTPYQALDSM | |
| | DDAWSFRELKAFVRESKTLSNKKKEYLLTEEDISKFDVRKKFI | |
| | ERNLVDTRYASRVVLNALQEHFRAHKIDTKVSVVRGQFTSQL | |
| | RRHWGIEKTRDTYHHHAVDALIIAASSQLNLWKKQKNTLVS | |
| | YSEDQLLDIETGELISDDEYKESVFKAPYQHFVDTLKSKEFED | |
| | SILFSYQVDSKFNRKISDATIYATRQAKVGKDKADETYVLGKI | |
| | KDIYTQDGYDAFMKIYKKDKSKFLMYRHDPQTFEKVIEPILEN | |
| | YPNKQINEKGKEVPCNPFLKYKEEHGYIRKYSKKGNGPEIKSL | |
| | KYYDSKLGNHIDITPKDSNNKVVLQSVSPWRADVYFNKTTGK | |
| | YEILGLKYADLQFEKGTGTYKISQEKYNDIKKKEGVDSDSEFK | |
| | FTLYKNDLLLVKDTETKEQQLFRFLSRTMPKQKHYVELKPYD | |
| | KQKFEGGEALIKVLGNVANSGQCKKGLGKSNISIYKVRTDVL | |
| | GNQHIIKNEGDKPKLDF | |
| St3Cas9 | MTKPYSIGLDIGTNSVGWAVTTDNYKVPSKKMKVLGNTSKK | SEQ ID |
| | YIKKNLLGVLLFDSGITAEGRRLKRTARRRYTRRRNRILYLQEI | NO. |
| | FSTEMATLDDAFFQRLDDSFLVPDDKRDSKYPIFGNLVEEKA | 314 |
| | YHDEFPTIYHLRKYLADSTKKADLRLVYLALAHMIKYRGHFL | |
| | IEGEFNSKNNDIQKNFQDFLDTYNAIFESDLSLENSKQLEEIVK | |
| | DKISKLEKKDRILKLFPGEKNSGIFSEFLKLIVGNQADFRKCFN | |
| | LDEKASLHFSKESYDEDLETLLGYIGDDYSDVFLKAKKLYDAI | |
| | LLSGFLTVTDNETEAPLSSAMIKRYNEHKEDLALLKEYIRNISL | |
| | KTYNEVFKDDTKNGYAGYIDGKTNQEDFYVYLKKLLAEFEG | |
| | ADYFLEKIDREDFLRKQRTFDNGSIPYQIHLQEMRAILDKQAK | |
| | FYPFLAKNKERIEKILTFRIPYYVGPLARGNSDFAWSIRKRNEK | |
| | ITPWNFEDVIDKESSAEAFINRMTSFDLYLPEEKVLPKHSLLYE | |
| | TFNVYNELTKVRFIAESMRDYQFLDSKQKKDIVRLYFKDKRK | |
| | VTDKDIIEYLHAIYGYDGIELKGIEKQFNSSLSTYHDLLNIINDK | |
| | EFLDDSSNEAIIEEIIHTLTIFEDREMIKQRLSKFENIFDKSVLKK | |
| | LSRRHYTGWGKLSAKLINGIRDEKSGNTILDYLIDDGISNRNF | |
| | MQLIHDDALSFKKKIQKAQIIGDEDKGNIKEVVKSLPGSPAIK | |
| | KGILQSIKIVDELVKVMGGRKPESIVVEMARENQYTNQGKSN | |
| | SQQRLKRLEKSLKELGSKILKENIPAKLSKIDNNALQNDRLYL | |
| | YYLQNGKDMYTGDDLDIDRLSNYDIDHIIPQAFLKDNSIDNKV | |
| | LVSSASNRGKSDDVPSLEVVKKRKTFWYQLLKSKLISQRKFD | |
| | NLTKAERGGLSPEDKAGFIQRQLVETRQITKHVARLLDEKFN | |
| | NKKDENNRAVRTVKIITLKSTLVSQFRKDFELYKVREINDFHH | |
| | AHDAYLNAVVASALLKKYPKLEPEFVYGDYPKYNSFRERKS | |
| | ATEKVYFYSNIMNIFKKSISLADGRVIERPLIEVNEETGESVWN | |
| | KESDLATVRRVLSYPQVNVVKKVEEQNHGLDRGKPKGLFNA | |
| | NLSSKPKPNSNENLVGAKEYLDPKKYGGYAGISNSFTVLVKG | |
| | TIEKGAKKKITNVLEFQGISILDRINYRKDKLNFLLEKGYKDIE | |
| | LIIELPKYSLFELSDGSRRMLASILSTNNKRGEIHKGNQIFLSQK | |
| | FVKLLYHAKRISNTINENHRKYVENHKKEFEELFYYILEFNEN | |
| | YVGAKKNGKLLNSAFQSWQNHSIDELCSSFIGPTGSERKGLFE | |
| | LTSRGSAADFEFLGVKIPRYRDYTPSSLLKDATLIHQSVTGLYE | |
| | TRIDLAKLGEG | |
| NmCas9 | MAAFKPNSINYILGLDIGIASVGWAMVEIDEEENPIRLIDLGVR | SEQ ID |
| | VFERAEVPKTGDSLAMARRLARSVRRLTRRRAHRLLRTRRLL | NO. |
| | KREGVLQAANFDENGLIKSLPNTPWQLRAAALDRKLTPLEWS | 315 |
| | AVLLHLIKHRGYLSQRKNEGETADKELGALLKGVAGNAHAL | |
| | QTGDFRTPAELALNKFEKESGHIRNQRSDYSHTFSRKDLQAEL | |
| | ILLFEKQKEFGNPHVSGGLKEGIETLLMTQRPALSGDAVQKM | |
| | LGHCTFEPAEPKAAKNTYTAERFIWLTKLNNLRILEQGSERPL | |
| | TDTERATLMDEPYRKSKLTYAQARKLLGLEDTAFFKGLRYG | |
| | KDNAEASTLMEMKAYHAISRALEKEGLKDKKSPLNLSPELQD | |
| | EIGTAFSLFKTDEDITGRLKDRIQPEILEALLKHISFDKFVQISLK | |
| | ALRRIVPLMEQGKRYDEACAEIYGDHYGKKNTEEKIYLPPIPA | |
| | DEIRNPVVLRALSQARKVINGVVRRYGSPARIHIETAREVGKS | |
| | FKDRKEIEKRQEENRKDREKAAAKFREYFPNFVGEPKSKDILK | |
| | LRLYEQQHGKCLYSGKEINLGRLNEKGYVEIDHALPFSRTWD | |
| | DSFNNKVLVLGSENQNKGNQTPYEYFNGKDNSREWQEFKAR | |
| | VETSRFPRSKKQRILLQKFDEDGFKERNLNDTRYVNRFLCQFV | |
| | ADRMRLTGKGKKRVFASNGQITNLLRGFWGLRKVRAENDRH | |
| | HALDAVVVACSTVAMQQKITRFVRYKEMNAFDGKTIDKETG | |
| | EVLHQKTHFPQPWEFFAQEVMIRVFGKPDGKPEFEEADTLEK | |
| | LRTLLAEKLSSRPEAVHEYVTPLFVSRAPNRKMSGQGHMETV | |
| | KSAKRLDEGVSVLRVPLTQLKLKDLEKMVNREREPKLYEAL | |
| | KARLEAHKDDPAKAFAEPFYKYDKAGNRTQQVKAVRVEQV | |
| | QKTGVWVRNHNGIADNATMVRVDVFEKGDKYYLVPIYSWQ | |
| | VAKGILPDRAVVQGKDEEDWQLIDDSFNFKFSLHPNDLVEVIT | |
| | KKARMFGYFASCHRGTGNINIRIHDLDHKIGKNGILEGIGVKT | |
| | ALSFQKYQIDELGKEIRPCRLKKRPPVR | |
| SaCas9 | MKRNYILGLDIGITSVGYGIIDYETRDVIDAGVRLFKEANVEN | SEQ ID |
| | NEGRRSKRGARRLKRRRRHRIQRVKKLLFDYNLLTDHSELSGI | NO. |
| | NPYEARVKGLSQKLSEEEFSAALLHLAKRRGVHNVNEVEEDT | 316 |
| | GNELSTKEQISRNSKALEEKYVAELQLERLKKDGEVRGSINRF | |
| | KTSDYVKEAKQLLKVQKAYHQLDQSFIDTYIDLLETRRTYYE | |
| | GPGEGSPFGWKDIKEWYEMLMGHCTYFPEELRSVKYAYNAD | |
| | LYNALNDLNNLVITRDENEKLEYYEKFQIIENVFKQKKKPTLK | |
| | QIAKEILVNEEDIKGYRVTSTGKPEFTNLKVYHDIKDITARKEII | |
| | ENAELLDQIAKILTIYQSSEDIQEELTNLNSELTQEEIEQISNLK | |
| | GYTGTHNLSLKAINLILDELWHTNDNQIAIFNRLKLVPKKVDL | |
| | SQQKEIPTTLVDDFILSPVVKRSFIQSIKVINAIIKKYGLPNDIIIE | |
| | LAREKNSKDAQKMINEMQKRNRQTNERIEEIIRTTGKENAKY | |
| | LIEKIKLHDMQEGKCLYSLEAIPLEDLLNNPFNYEVDHIIPRSV | |
| | SFDNSFNNKVLVKQEENSKKGNRTPFQYLSSSDSKISYETFKK | |
| | HILNLAKGKGRISKTKKEYLLEERDINRFSVQKDFINRNLVDT | |
| | RYATRGLMNLLRSYFRVNNLDVKVKSINGGFTSFLRRKWKF | |
| | KKERNKGYKHHAEDALIIANADFIFKEWKKLDKAKKVMENQ | |
| | MFEEKQAESMPEIETEQEYKEIFITPHQIKHIKDFKDYKYSHRV | |
| | DKKPNRELINDTLYSTRKDDKGNTLIVNNLNGLYDKDNDKLK | |
| | KLINKSPEKLLMYHHDPQTYQKLKLIMEQYGDEKNPLYKYYE | |
| | ETGNYLTKYSKKDNGPVIKKIKYYGNKLNAHLDITDDYPNSR | |
| | NKVVKLSLKPYRFDVYLDNGVYKFVTVKNLDVIKKENYYEV | |
| | NSKCYEEAKKLKKISNQAEFIASFYNNDLIKINGELYRVIGVN | |
| | NDLLNRIEVNMIDITYREYLENMNDKRPPRIIKTIASKTQSIKK | |
| | YSTDILGNLYEVKSKKHPQIIKKG | |
| AsCpf1 | MTQFEGFTNLYQVSKTLRFELIPQGKTLKHIQEQGFIEEDKAR | SEQ ID |
| | NDHYKELKPIIDRIYKTYADQCLQLVQLDWENLSAAIDSYRK | NO. |
| | EKTEETRNALIEEQATYRNAIHDYFIGRTDNLTDAINKRHAEIY | 317 |
| | KGLFKAELFNGKVLKQLGTVTTTEHENALLRSFDKFTTYFSGF | |
| | YENRKNVFSAEDISTAIPHRIVQDNFPKFKENCHIFTRLITAVPS | |
| | LREHFENVKKAIGIFVSTSIEEVFSFPFYNQLLTQTQIDLYNQLL | |
| | GGISREAGTEKIKGLNEVLNLAIQKNDETAHIIASLPHRFIPLFK | |
| | QILSDRNTLSFILEEFKSDEEVIQSFCKYKTLLRNENVLETAEA | |
| | LFNELNSIDLTHIFISHKKLETISSALCDHWDTLRNALYERRISE | |
| | LTGKITKSAKEKVQRSLKHEDINLQEIISAAGKELSEAFKQKTS | |
| | EILSHAHAALDQPLPTTLKKQEEKEILKSQLDSLLGLYHLLDW | |
| | FAVDESNEVDPEFSARLTGIKLEMEPSLSFYNKARNYATKKPY | |
| | SVEKFKLNFQMPTLASGWDVNKEKNNGAILFVKNGLYYLGI | |
| | MPKQKGRYKALSFEPTEKTSEGFDKMYYDYFPDAAKMIPKCS | |
| | TQLKAVTAHFQTHTTPILLSNNFIEPLEITKEIYDLNNPEKEPKK | |
| | FQTAYAKKTGDQKGYREALCKWIDFTRDFLSKYTKTTSIDLS | |
| | SLRPSSQYKDLGEYYAELNPLLYHISFQRIAEKEIMDAVETGK | |
| | LYLFQIYNKDFAKGHHGKPNLHTLYWTGLFSPENLAKTSIKL | |
| | NGQAELFYRPKSRMKRMAHRLGEKMLNKKLKDQKTPIPDTL | |
| | YQELYDYVNHRLSHDLSDEARALLPNVITKEVSHEIIKDRRFT | |
| | SDKFFFHVPITLNYQAANSPSKFNQRVNAYLKEHPETPIIGIDR | |
| | GERNLIYITVIDSTGKILEQRSLNTIQQFDYQKKLDNREKERVA | |
| | ARQAWSVVGTIKDLKQGYLSQVIHEIVDLMIHYQAVVVLENL | |
| | NFGFKSKRTGIAEKAVYQQFEKMLIDKLNCLVLKDYPAEKVG | |
| | GVLNPYQLTDQFTSFAKMGTQSGFLFYVPAPYTSKIDPLTGFV | |
| | DPFVWKTIKNHESRKHFLEGFDFLHYDVKTGDFILHFKMNRN | |
| | LSFQRGLPGFMPAWDIVFEKNETQFDAKGTPFIAGKRIVPVIE | |
| | NHRFTGRYRDLYPANELIALLEEKGIVFRDGSNILPKLLENDD | |
| | SHAIDTMVALIRSVLQMRNSNAATGEDYINSPVRDLNGVCFD | |
| | SRFQNPEWPMDADANGAYHIALKGQLLLNHLKESKDLKLQN | |
| | GISNQDWLAYIQELRN | |
| LbCpf1 | MAASKLEKFTNCYSLSKTLRFKAIPVGKTQENIDNKRLLVEDE | SEQ ID |
| | KRAEDYKGVKKLLDRYYLSFINDVLHSIKLKNLNNYISLFRKK | NO. |
| | TRTEKENKELENLEINLRKEIAKAFKGAAGYKSLFKKDIIETIL | 318 |
| | PEAADDKDEIALVNSFNGFTTAFTGFFDNRENMFSEEAKSTSI | |
| | AFRCINENLTRYISNMDIFEKVDAIFDKHEVQEIKEKILNSDYD | |
| | VEDFFEGEFFNFVLTQEGIDVYNAIIGGFVTESGEKIKGLNEYI | |
| | NLYNAKTKQALPKFKPLYKQVLSDRESLSFYGEGYTSDEEVL | |
| | EVFRNTLNKNSEIFSSIKKLEKLFKNFDEYSSAGIFVKNGPAIST | |
| | ISKDIFGEWNLIRDKWNAEYDDIHLKKKAVVTEKYEDDRRKS | |
| | FKKIGSFSLEQLQEYADADLSVVEKLKEIIIQKVDEIYKVYGSS | |
| | EKLFDADFVLEKSLKKNDAVVAIMKDLLDSVKSFENYIKAFF | |
| | GEGKETNRDESFYGDFVLAYDILLKVDHIYDAIRNYVTQKPY | |
| | SKDKFKLYFQNPQFMGGWDKDKETDYRATILRYGSKYYLAI | |
| | MDKKYAKCLQKIDKDDVNGNYEKINYKLLPGPNKMLPKVFF | |
| | SKKWMAYYNPSEDIQKIYKNGTFKKGDMFNLNDCHKLIDFF | |
| | KDSISRYPKWSNAYDFNFSETEKYKDIAGFYREVEEQGYKVS | |
| | FESASKKEVDKLVEEGKLYMFQIYNKDFSDKSHGTPNLHTMY | |
| | FKLLFDENNHGQIRLSGGAELFMRRASLKKEELVVHPANSPIA | |
| | NKNPDNPKKTTTLSYDVYKDKRFSEDQYELHIPIAINKCPKNIF | |
| | KINTEVRVLLKHDDNPYVIGIDRGERNLLYIVVVDGKGNIVEQ | |
| | YSLNEIINNFNGIRIKTDYHSLLDKKEKERFEARQNWTSIENIK | |
| | ELKAGYISQVVHKICELVEKYDAVIALEDLNSGFKNSRVKVE | |
| | KQVYQKFEKMLIDKLNYMVDKKSNPCATGGALKGYQITNKF | |
| | ESFKSMSTQNGFIFYIPAWLTSKIDPSTGFVNLLKTKYTSIADS | |
| | KKFISSFDRIMYVPEEDLFEFALDYKNFSRTDADYIKKWKLYS | |
| | YGNRIRIFAAAKKNNVFAWEEVCLTSAYKELFNKYGINYQQG | |
| | DIRALLCEQSDKAFYSSFMALMSLMLQMRNSITGRTDVDFLIS | |
| | PVKNSDGIFYDSRNYEAQENAILPKNADANGAYNIARKVLWA | |
| | IGQFKKAEDEKLDKVKIAISNKEWLEYAQTSVK | |
| FnCpf1 | MSIYQEFVNKYSLSKTLRFELIPQGKTLENIKARGLILDDEKRA | SEQ ID |
| | KDYKKAKQIIDKYHQFFIEEILSSVCISEDLLQNYSDVYFKLKK | NO. |
| | SDDDNLQKDFKSAKDTIKKQISEYIKDSEKFKNLFNQNLIDAK | 319 |
| | KGQESDLILWLKQSKDNGIELFKANSDITDIDEALEIIKSFKGW | |
| | TTYFKGFHENRKNVYSSNDIPTSIIYRIVDDNLPKFLENKAKYE | |
| | SLKDKAPEAINYEQIKKDLAEELTFDIDYKTSEVNQRVFSLDE | |
| | VFEIANFNNYLNQSGITKFNTIIGGKFVNGENTKRKGINEYINL | |
| | YSQQINDKTLKKYKMSVLFKQILSDTESKSFVIDKLEDDSDVV | |
| | TTMQSFYEQIAAFKTVEEKSIKETLSLLFDDLKAQKLDLSKIYF | |
| | KNDKSLTDLSQQVFDDYSVIGTAVLEYITQQIAPKNLDNPSKK | |
| | EQELIAKKTEKAKYLSLETIKLALEEFNKHRDIDKQCRFEEILA | |
| | NFAAIPMIFDEIAQNKDNLAQISIKYQNQGKKDLLQASAEDDV | |
| | KAIKDLLDQTNNLLHKLKIFHISQSEDKANILDKDEHFYLVFE | |
| | ECYFELANIVPLYNKIRNYITQKPYSDEKFKLNFENSTLANGW | |
| | DKNKEPDNTAILFIKDDKYYLGVMNKKNNKIFDDKAIKENKG | |
| | EGYKKIVYKLLPGANKMLPKVFFSAKSIKFYNPSEDILRIRNHS | |
| | THTKNGSPQKGYEKFEFNIEDCRKFIDFYKQSISKHPEWKDFG | |
| | FRFSDTQRYNSIDEFYREVENQGYKLTFENISESYIDSVVNQG | |
| | KLYLFQIYNKDFSAYSKGRPNLHTLYWKALFDERNLQDVVY | |
| | KLNGEAELFYRKQSIPKKITHPAKEAIANKNKDNPKKESVFEY | |
| | DLIKDKRFTEDKFFFHCPITINFKSSGANKFNDEINLLLKEKAN | |
| | DVHILSIDRGERHLAYYTLVDGKGNIIKQDTFNIIGNDRMKTN | |
| | YHDKLAAIEKDRDSARKDWKKINNIKEMKEGYLSQVVHEIA | |
| | KLVIEYNAIVVFEDLNFGFKRGRFKVEKQVYQKLEKMLIEKL | |
| | NYLVFKDNEFDKTGGVLRAYQLTAPFETFKKMGKQTGIIYYV | |
| | PAGFTSKICPVTGFVNQLYPKYESVSKSQEFFSKFDKICYNLD | |
| | KGYFEFSFDYKNFGDKAAKGKWTIASFGSRLINFRNSDKNHN | |
| | WDTREVYPTKELEKLLKDYSIEYGHGECIKAAICGESDKKFFA | |
| | KLTSVLNTILQMRNSKTGTELDYLISPVADVNGNFFDSRQAPK | |
| | NMPQDADANGAYHIGLKGLMLLGRIKNNQEGKKLNLVIKNE | |
| | EYFEFVQNRNN | |
| VQRCas9 | MDKKYSIGLDIGTNSVGWAVITDEYKVPSKKFKVLGNTDRHS | SEQ ID |
| | IKKNLIGALLFDSGETAEATRLKRTARRRYTRRKNRICYLQEIF | NO. |
| | SNEMAKVDDSFFHRLEESFLVEEDKKHERHPIFGNIVDEVAYH | 320 |
| | EKYPTIYHLRKKLVDSTDKADLRLIYLALAHMIKFRGHFLIEG | |
| | DLNPDNSDVDKLFIQLVQTYNQLFEENPINASGVDAKAILSAR | |
| | LSKSRRLENLIAQLPGEKKNGLFGNLIALSLGLTPNFKSNFDLA | |
| | EDAKLQLSKDTYDDDLDNLLAQIGDQYADLFLAAKNLSDAIL | |
| | LSDILRVNTEITKAPLSASMIKRYDEHHQDLTLLKALVRQQLP | |
| | EKYKEIFFDQSKNGYAGYIDGGASQEEFYKFIKPILEKMDGTE | |
| | ELLVKLNREDLLRKQRTFDNGSIPHQIHLGELHAILRRQEDFY | |
| | PFLKDNREKIEKILTFRIPYYVGPLARGNSRFAWMTRKSEETIT | |
| | PWNFEEVVDKGASAQSFIERMTNFDKNLPNEKVLPKHSLLYE | |
| | YFTVYNELTKVKYVTEGMRKPAFLSGEQKKAIVDLLFKTNRK | |
| | VTVKQLKEDYFKKIECFDSVEISGVEDRFNASLGTYHDLLKIIK | |
| | DKDFLDNEENEDILEDIVLTLTLFEDREMIEERLKTYAHLFDD | |
| | KVMKQLKRRRYTGWGRLSRKLINGIRDKQSGKTILDFLKSDG | |
| | FANRNFMQLIHDDSLTFKEDIQKAQVSGQGDSLHEHIANLAG | |
| | SPAIKKGILQTVKVVDELVKVMGRHKPENIVIEMARENQTTQ | |
| | KGQKNSRERMKRIEEGIKELGSQILKEHPVENTQLQNEKLYLY | |
| | YLQNGRDMYVDQELDINRLSDYDVDHIVPQSFLKDDSIDNKV | |
| | LTRSDKNRGKSDNVPSEEVVKKMKNYWRQLLNAKLITQRKF | |
| | DNLTKAERGGLSELDKAGFIKRQLVETRQITKHVAQILDSRM | |
| | NTKYDENDKLIREVKVITLKSKLVSDFRKDFQFYKVREINNYH | |
| | HAHDAYLNAVVGTALIKKYPKLESEFVYGDYKVYDVRKMIA | |
| | KSEQEIGKATAKYFFYSNIMNFFKTEITLANGEIRKRPLIETNG | |
| | ETGEIVWDKGRDFATVRKVLSMPQVNIVKKTEVQTGGFSKES | |
| | ILPKRNSDKLIARKKDWDPKKYGGFVSPTVAYSVLVVAKVEK | |
| | GKSKKLKSVKELLGITIMERSSFEKNPIDFLEAKGYKEVKKDLI | |
| | IKLPKYSLFELENGRKRMLASAGELQKGNELALPSKYVNFLY | |
| | LASHYEKLKGSPEDNEQKQLFVEQHKHYLDEIIEQISEFSKRVI | |
| | LADANLDKVLSAYNKHRDKPIREQAENIIHLFTLTNLGAPAAF | |
| | KYFDTTIDRKQYRSTKEVLDATLIHQSITGLYETRIDLSQLGGD | |
| EQRCas9 | MDKKYSIGLDIGTNSVGWAVITDEYKVPSKKFKVLGNTDRHS | SEQ ID |
| | IKKNLIGALLFDSGETAEATRLKRTARRRYTRRKNRICYLQEIF | NO. |
| | SNEMAKVDDSFFHRLEESFLVEEDKKHERHPIFGNIVDEVAYH | 321 |
| | EKYPTIYHLRKKLVDSTDKADLRLIYLALAHMIKFRGHFLIEG | |
| | DLNPDNSDVDKLFIQLVQTYNQLFEENPINASGVDAKAILSAR | |
| | LSKSRRLENLIAQLPGEKKNGLFGNLIALSLGLTPNFKSNFDLA | |
| | EDAKLQLSKDTYDDDLDNLLAQIGDQYADLFLAAKNLSDAIL | |
| | LSDILRVNTEITKAPLSASMIKRYDEHHQDLTLLKALVRQQLP | |
| | EKYKEIFFDQSKNGYAGYIDGGASQEEFYKFIKPILEKMDGTE | |
| | ELLVKLNREDLLRKQRTFDNGSIPHQIHLGELHAILRRQEDFY | |
| | PFLKDNREKIEKILTFRIPYYVGPLARGNSRFAWMTRKSEETIT | |
| | PWNFEEVVDKGASAQSFIERMTNFDKNLPNEKVLPKHSLLYE | |
| | YFTVYNELTKVKYVTEGMRKPAFLSGEQKKAIVDLLFKTNRK | |
| | VTVKQLKEDYFKKIECFDSVEISGVEDRFNASLGTYHDLLKIIK | |
| | DKDFLDNEENEDILEDIVLTLTLFEDREMIEERLKTYAHLFDD | |
| | KVMKQLKRRRYTGWGRLSRKLINGIRDKQSGKTILDFLKSDG | |
| | FANRNFMQLIHDDSLTFKEDIQKAQVSGQGDSLHEHIANLAG | |
| | SPAIKKGILQTVKVVDELVKVMGRHKPENIVIEMARENQTTQ | |
| | KGQKNSRERMKRIEEGIKELGSQILKEHPVENTQLQNEKLYLY | |
| | YLQNGRDMYVDQELDINRLSDYDVDHIVPQSFLKDDSIDNKV | |
| | LTRSDKNRGKSDNVPSEEVVKKMKNYWRQLLNAKLITQRKF | |
| | DNLTKAERGGLSELDKAGFIKRQLVETRQITKHVAQILDSRM | |
| | NTKYDENDKLIREVKVITLKSKLVSDFRKDFQFYKVREINNYH | |
| | HAHDAYLNAVVGTALIKKYPKLESEFVYGDYKVYDVRKMIA | |
| | KSEQEIGKATAKYFFYSNIMNFFKTEITLANGEIRKRPLIETNG | |
| | ETGEIVWDKGRDFATVRKVLSMPQVNIVKKTEVQTGGFSKES | |
| | ILPKRNSDKLIARKKDWDPKKYGGFESPTVAYSVLVVAKVEK | |
| | GKSKKLKSVKELLGITIMERSSFEKNPIDFLEAKGYKEVKKDLI | |
| | IKLPKYSLFELENGRKRMLASAGELQKGNELALPSKYVNFLY | |
| | LASHYEKLKGSPEDNEQKQLFVEQHKHYLDEIIEQISEFSKRVI | |
| | LADANLDKVLSAYNKHRDKPIREQAENIIHLFTLTNLGAPAAF | |
| | KYFDTTIDRKQYRSTKEVLDATLIHQSITGLYETRIDLSQLGGD | |
| VRERCas9 | MDKKYSIGLDIGTNSVGWAVITDEYKVPSKKFKVLGNTDRHS | SEQ ID |
| | IKKNLIGALLFDSGETAEATRLKRTARRRYTRRKNRICYLQEIF | NO. |
| | SNEMAKVDDSFFHRLEESFLVEEDKKHERHPIFGNIVDEVAYH | 322 |
| | EKYPTIYHLRKKLVDSTDKADLRLIYLALAHMIKFRGHFLIEG | |
| | DLNPDNSDVDKLFIQLVQTYNQLFEENPINASGVDAKAILSAR | |
| | LSKSRRLENLIAQLPGEKKNGLFGNLIALSLGLTPNFKSNFDLA | |
| | EDAKLQLSKDTYDDDLDNLLAQIGDQYADLFLAAKNLSDAIL | |
| | LSDILRVNTEITKAPLSASMIKRYDEHHQDLTLLKALVRQQLP | |
| | EKYKEIFFDQSKNGYAGYIDGGASQEEFYKFIKPILEKMDGTE | |
| | ELLVKLNREDLLRKQRTFDNGSIPHQIHLGELHAILRRQEDFY | |
| | PFLKDNREKIEKILTFRIPYYVGPLARGNSRFAWMTRKSEETIT | |
| | PWNFEEVVDKGASAQSFIERMTNFDKNLPNEKVLPKHSLLYE | |
| | YFTVYNELTKVKYVTEGMRKPAFLSGEQKKAIVDLLFKTNRK | |
| | VTVKQLKEDYFKKIECFDSVEISGVEDRFNASLGTYHDLLKIIK | |
| | DKDFLDNEENEDILEDIVLTLTLFEDREMIEERLKTYAHLFDD | |
| | KVMKQLKRRRYTGWGRLSRKLINGIRDKQSGKTILDFLKSDG | |
| | FANRNFMQLIHDDSLTFKEDIQKAQVSGQGDSLHEHIANLAG | |
| | SPAIKKGILQTVKVVDELVKVMGRHKPENIVIEMARENQTTQ | |
| | KGQKNSRERMKRIEEGIKELGSQILKEHPVENTQLQNEKLYLY | |
| | YLQNGRDMYVDQELDINRLSDYDVDHIVPQSFLKDDSIDNKV | |
| | LTRSDKNRGKSDNVPSEEVVKKMKNYWRQLLNAKLITQRKF | |
| | DNLTKAERGGLSELDKAGFIKRQLVETRQITKHVAQILDSRM | |
| | NTKYDENDKLIREVKVITLKSKLVSDFRKDFQFYKVREINNYH | |
| | HAHDAYLNAVVGTALIKKYPKLESEFVYGDYKVYDVRKMIA | |
| | KSEQEIGKATAKYFFYSNIMNFFKTEITLANGEIRKRPLIETNG | |
| | ETGEIVWDKGRDFATVRKVLSMPQVNIVKKTEVQTGGFSKES | |
| | ILPKRNSDKLIARKKDWDPKKYGGFVSPTVAYSVLVVAKVEK | |
| | GKSKKLKSVKELLGITIMERSSFEKNPIDFLEAKGYKEVKKDLI | |
| | IKLPKYSLFELENGRKRMLASARELQKGNELALPSKYVNFLY | |
| | LASHYEKLKGSPEDNEQKQLFVEQHKHYLDEIIEQISEFSKRVI | |
| | LADANLDKVLSAYNKHRDKPIREQAENIIHLFTLTNLGAPAAF | |
| | KYFDTTIDRKEYRSTKEVLDATLIHQSITGLYETRIDLSQLGGD | |
| Cas9- | MDKKYSIGLDIGTNSVGWAVITDEYKVPSKKFKVLGNTDRHS | SEQ ID |
| NG | IKKNLIGALLFDSGETAEATRLKRTARRRYTRRKNRICYLQEIF | NO. |
| | SNEMAKVDDSFFHRLEESFLVEEDKKHERHPIFGNIVDEVAYH | 323 |
| | EKYPTIYHLRKKLVDSTDKADLRLIYLALAHMIKFRGHFLIEG | |
| | DLNPDNSDVDKLFIQLVQTYNQLFEENPINASGVDAKAILSAR | |
| | LSKSRRLENLIAQLPGEKKNGLFGNLIALSLGLTPNFKSNFDLA | |
| | EDAKLQLSKDTYDDDLDNLLAQIGDQYADLFLAAKNLSDAIL | |
| | LSDILRVNTEITKAPLSASMIKRYDEHHQDLTLLKALVRQQLP | |
| | EKYKEIFFDQSKNGYAGYIDGGASQEEFYKFIKPILEKMDGTE | |
| | ELLVKLNREDLLRKQRTFDNGSIPHQIHLGELHAILRRQEDFY | |
| | PFLKDNREKIEKILTFRIPYYVGPLARGNSRFAWMTRKSEETIT | |
| | PWNFEEVVDKGASAQSFIERMTNFDKNLPNEKVLPKHSLLYE | |
| | YFTVYNELTKVKYVTEGMRKPAFLSGEQKKAIVDLLFKTNRK | |
| | VTVKQLKEDYFKKIECFDSVEISGVEDRFNASLGTYHDLLKIIK | |
| | DKDFLDNEENEDILEDIVLTLTLFEDREMIEERLKTYAHLFDD | |
| | KVMKQLKRRRYTGWGRLSRKLINGIRDKQSGKTILDFLKSDG | |
| | FANRNFMQLIHDDSLTFKEDIQKAQVSGQGDSLHEHIANLAG | |
| | SPAIKKGILQTVKVVDELVKVMGRHKPENIVIEMARENQTTQ | |
| | KGQKNSRERMKRIEEGIKELGSQILKEHPVENTQLQNEKLYLY | |
| | YLQNGRDMYVDQELDINRLSDYDVDHIVPQSFLKDDSIDNKV | |
| | LTRSDKNRGKSDNVPSEEVVKKMKNYWRQLLNAKLITQRKF | |
| | DNLTKAERGGLSELDKAGFIKRQLVETRQITKHVAQILDSRM | |
| | NTKYDENDKLIREVKVITLKSKLVSDFRKDFQFYKVREINNYH | |
| | HAHDAYLNAVVGTALIKKYPKLESEFVYGDYKVYDVRKMIA | |
| | KSEQEIGKATAKYFFYSNIMNFFKTEITLANGEIRKRPLIETNG | |
| | ETGEIVWDKGRDFATVRKVLSMPQVNIVKKTEVQTGGFSKES | |
| | IRPKRNSDKLIARKKDWDPKKYGGFVSPTVAYSVLVVAKVEK | |
| | GKSKKLKSVKELLGITIMERSSFEKNPIDFLEAKGYKEVKKDLI | |
| | IKLPKYSLFELENGRKRMLASARFLQKGNELALPSKYVNFLYL | |
| | ASHYEKLKGSPEDNEQKQLFVEQHKHYLDEIIEQISEFSKRVIL | |
| | ADANLDKVLSAYNKHRDKPIREQAENIIHLFTLTNLGAPRAFK | |
| | YFDTTIDRKVYRSTKEVLDATLIHQSITGLYETRIDLSQLGGD | |
| xCas9 | MDKKYSIGLDIGTNSVGWAVITDEYKVPSKKFKVLGNTDRHS | SEQ ID |
| | IKKNLIGALLFDSGETAEATRLKRTARRRYTRRKNRICYLQEIF | NO. |
| | SNEMAKVDDSFFHRLEESFLVEEDKKHERHPIFGNIVDEVAYH | 324 |
| | EKYPTIYHLRKKLVDSTDKADLRLIYLALAHMIKFRGHFLIEG | |
| | DLNPDNSDVDKLFIQLVQTYNQLFEENPINASGVDAKAILSAR | |
| | LSKSRRLENLIAQLPGEKKNGLFGNLIALSLGLTPNFKSNFDLA | |
| | EDTKLQLSKDTYDDDLDNLLAQIGDQYADLFLAAKNLSDAIL | |
| | LSDILRVNTEITKAPLSASMIKLYDEHHQDLTLLKALVRQQLP | |
| | EKYKEIFFDQSKNGYAGYIDGGASQEEFYKFIKPILEKMDGTE | |
| | ELLVKLNREDLLRKQRTFDNGIIPHQIHLGELHAILRRQEDFYP | |
| | FLKDNREKIEKILTFRIPYYVGPLARGNSRFAWMTRKSEETITP | |
| | WNFEKVVDKGASAQSFIERMTNFDKNLPNEKVLPKHSLLYEY | |
| | FTVYNELTKVKYVTEGMRKPAFLSGDQKKAIVDLLFKTNRK | |
| | VTVKQLKEDYFKKIECFDSVEISGVEDRFNASLGTYHDLLKIIK | |
| | DKDFLDNEENEDILEDIVLTLTLFEDREMIEERLKTYAHLFDD | |
| | KVMKQLKRRRYTGWGRLSRKLINGIRDKQSGKTILDFLKSDG | |
| | FANRNFIQLIHDDSLTFKEDIQKAQVSGQGDSLHEHIANLAGS | |
| | PAIKKGILQTVKVVDELVKVMGRHKPENIVIEMARENQTTQK | |
| | GQKNSRERMKRIEEGIKELGSQILKEHPVENTQLQNEKLYLYY | |
| | LQNGRDMYVDQELDINRLSDYDVDHIVPQSFLKDDSIDNKVL | |
| | TRSDKNRGKSDNVPSEEVVKKMKNYWRQLLNAKLITQRKFD | |
| | NLTKAERGGLSELDKAGFIKRQLVETRQITKHVAQILDSRMNT | |
| | KYDENDKLIREVKVITLKSKLVSDFRKDFQFYKVREINNYHH | |
| | AHDAYLNAVVGTALIKKYPKLESEFVYGDYKVYDVRKMIAK | |
| | SEQEIGKATAKYFFYSNIMNFFKTEITLANGEIRKRPLIETNGET | |
| | GEIVWDKGRDFATVRKVLSMPQVNIVKKTEVQTGGFSKESIL | |
| | PKRNSDKLIARKKDWDPKKYGGFDSPTVAYSVLVVAKVEKG | |
| | KSKKLKSVKELLGITIMERSSFEKNPIDFLEAKGYKEVKKDLII | |
| | KLPKYSLFELENGRKRMLASAGVLQKGNELALPSKYVNFLYL | |
| | ASHYEKLKGSPEDNEQKQLFVEQHKHYLDEIIEQISEFSKRVIL | |
| | ADANLDKVLSAYNKHRDKPIREQAENIIHLFTLTNLGAPAAFK | |
| | YFDTTIDRKRYTSTKEVLDATLIHQSITGLYETRIDLSQLGGD | |
| eCas9 | MDKKYSIGLDIGTNSVGWAVITDEYKVPSKKFKVLGNTDRHS | SEQ ID |
| | IKKNLIGALLFDSGETAEATRLKRTARRRYTRRKNRICYLQEIF | NO. |
| | SNEMAKVDDSFFHRLEESFLVEEDKKHERHPIFGNIVDEVAYH | 325 |
| | EKYPTIYHLRKKLVDSTDKADLRLIYLALAHMIKFRGHFLIEG | |
| | DLNPDNSDVDKLFIQLVQTYNQLFEENPINASGVDAKAILSAR | |
| | LSKSRRLENLIAQLPGEKKNGLFGNLIALSLGLTPNFKSNFDLA | |
| | EDAKLQLSKDTYDDDLDNLLAQIGDQYADLFLAAKNLSDAIL | |
| | LSDILRVNTEITKAPLSASMIKRYDEHHQDLTLLKALVRQQLP | |
| | EKYKEIFFDQSKNGYAGYIDGGASQEEFYKFIKPILEKMDGTE | |
| | ELLVKLNREDLLRKQRTFDNGSIPHQIHLGELHAILRRQEDFY | |
| | PFLKDNREKIEKILTFRIPYYVGPLARGNSRFAWMTRKSEETIT | |
| | PWNFEEVVDKGASAQSFIERMTNFDKNLPNEKVLPKHSLLYE | |
| | YFTVYNELTKVKYVTEGMRKPAFLSGEQKKAIVDLLFKTNRK | |
| | VTVKQLKEDYFKKIECFDSVEISGVEDRFNASLGTYHDLLKIIK | |
| | DKDFLDNEENEDILEDIVLTLTLFEDREMIEERLKTYAHLFDD | |
| | KVMKQLKRRRYTGWGRLSRKLINGIRDKQSGKTILDFLKSDG | |
| | FANRNFMQLIHDDSLTFKEDIQKAQVSGQGDSLHEHIANLAG | |
| | SPAIKKGILQTVKVVDELVKVMGRHKPENIVIEMARENQTTQ | |
| | KGQKNSRERMKRIEEGIKELGSQILKEHPVENTQLQNEKLYLY | |
| | YLQNGRDMYVDQELDINRLSDYDVDHIVPQSFLADDSIDNKV | |
| | LTRSDKNRGKSDNVPSEEVVKKMKNYWRQLLNAKLITQRKF | |
| | DNLTKAERGGLSELDKAGFIKRQLVETRQITKHVAQILDSRM | |
| | NTKYDENDKLIREVKVITLKSKLVSDFRKDFQFYKVREINNYH | |
| | HAHDAYLNAVVGTALIKKYPALESEFVYGDYKVYDVRKMIA | |
| | KSEQEIGKATAKYFFYSNIMNFFKTEITLANGEIRKAPLIETNG | |
| | ETGEIVWDKGRDFATVRKVLSMPQVNIVKKTEVQTGGFSKES | |
| | ILPKRNSDKLIARKKDWDPKKYGGFDSPTVAYSVLVVAKVEK | |
| | GKSKKLKSVKELLGITIMERSSFEKNPIDFLEAKGYKEVKKDLI | |
| | IKLPKYSLFELENGRKRMLASAGELQKGNELALPSKYVNFLY | |
| | LASHYEKLKGSPEDNEQKQLFVEQHKHYLDEIIEQISEFSKRVI | |
| | LADANLDKVLSAYNKHRDKPIREQAENIIHLFTLTNLGAPAAF | |
| | KYFDTTIDRKRYTSTKEVLDATLIHQSITGLYETRIDLSQLGGD | |
| SpCas9- | MDKKYSIGLDIGTNSVGWAVITDEYKVPSKKFKVLGNTDRHS | SEQ ID |
| HF1 | IKKNLIGALLFDSGETAEATRLKRTARRRYTRRKNRICYLQEIF | NO. |
| | SNEMAKVDDSFFHRLEESFLVEEDKKHERHPIFGNIVDEVAYH | 326 |
| | EKYPTIYHLRKKLVDSTDKADLRLIYLALAHMIKFRGHFLIEG | |
| | DLNPDNSDVDKLFIQLVQTYNQLFEENPINASGVDAKAILSAR | |
| | LSKSRRLENLIAQLPGEKKNGLFGNLIALSLGLTPNFKSNFDLA | |
| | EDAKLQLSKDTYDDDLDNLLAQIGDQYADLFLAAKNLSDAIL | |
| | LSDILRVNTEITKAPLSASMIKRYDEHHQDLTLLKALVRQQLP | |
| | EKYKEIFFDQSKNGYAGYIDGGASQEEFYKFIKPILEKMDGTE | |
| | ELLVKLNREDLLRKQRTFDNGSIPHQIHLGELHAILRRQEDFY | |
| | PFLKDNREKIEKILTFRIPYYVGPLARGNSRFAWMTRKSEETIT | |
| | PWNFEEVVDKGASAQSFIERMTAFDKNLPNEKVLPKHSLLYE | |
| | YFTVYNELTKVKYVTEGMRKPAFLSGEQKKAIVDLLFKTNRK | |
| | VTVKQLKEDYFKKIECFDSVEISGVEDRFNASLGTYHDLLKIIK | |
| | DKDFLDNEENEDILEDIVLTLTLFEDREMIEERLKTYAHLFDD | |
| | KVMKQLKRRRYTGWGALSRKLINGIRDKQSGKTILDFLKSDG | |
| | FANRNFMALIHDDSLTFKEDIQKAQVSGQGDSLHEHIANLAG | |
| | SPAIKKGILQTVKVVDELVKVMGRHKPENIVIEMARENQTTQ | |
| | KGQKNSRERMKRIEEGIKELGSQILKEHPVENTQLQNEKLYLY | |
| | YLQNGRDMYVDQELDINRLSDYDVDHIVPQSFLKDDSIDNKV | |
| | LTRSDKNRGKSDNVPSEEVVKKMKNYWRQLLNAKLITQRKF | |
| | DNLTKAERGGLSELDKAGFIKRQLVETRAITKHVAQILDSRM | |
| | NTKYDENDKLIREVKVITLKSKLVSDFRKDFQFYKVREINNYH | |
| | HAHDAYLNAVVGTALIKKYPKLESEFVYGDYKVYDVRKMIA | |
| | KSEQEIGKATAKYFFYSNIMNFFKTEITLANGEIRKRPLIETNG | |
| | ETGEIVWDKGRDFATVRKVLSMPQVNIVKKTEVQTGGFSKES | |
| | ILPKRNSDKLIARKKDWDPKKYGGFDSPTVAYSVLVVAKVEK | |
| | GKSKKLKSVKELLGITIMERSSFEKNPIDFLEAKGYKEVKKDLI | |
| | IKLPKYSLFELENGRKRMLASAGELQKGNELALPSKYVNFLY | |
| | LASHYEKLKGSPEDNEQKQLFVEQHKHYLDEIIEQISEFSKRVI | |
| | LADANLDKVLSAYNKHRDKPIREQAENIIHLFTLTNLGAPAAF | |
| | KYFDTTIDRKRYTSTKEVLDATLIHQSITGLYETRIDLSQLGGD | |
| HypaCas9 | MDKKYSIGLDIGTNSVGWAVITDEYKVPSKKFKVLGNTDRHS | SEQ ID |
| | IKKNLIGALLFDSGETAEATRLKRTARRRYTRRKNRICYLQEIF | NO. |
| | SNEMAKVDDSFFHRLEESFLVEEDKKHERHPIFGNIVDEVAYH | 327 |
| | EKYPTIYHLRKKLVDSTDKADLRLIYLALAHMIKFRGHFLIEG | |
| | DLNPDNSDVDKLFIQLVQTYNQLFEENPINASGVDAKAILSAR | |
| | LSKSRRLENLIAQLPGEKKNGLFGNLIALSLGLTPNFKSNFDLA | |
| | EDAKLQLSKDTYDDDLDNLLAQIGDQYADLFLAAKNLSDAIL | |
| | LSDILRVNTEITKAPLSASMIKRYDEHHQDLTLLKALVRQQLP | |
| | EKYKEIFFDQSKNGYAGYIDGGASQEEFYKFIKPILEKMDGTE | |
| | ELLVKLNREDLLRKQRTFDNGSIPHQIHLGELHAILRRQEDFY | |
| | PFLKDNREKIEKILTFRIPYYVGPLARGNSRFAWMTRKSEETIT | |
| | PWNFEEVVDKGASAQSFIERMTNFDKNLPNEKVLPKHSLLYE | |
| | YFTVYNELTKVKYVTEGMRKPAFLSGEQKKAIVDLLFKTNRK | |
| | VTVKQLKEDYFKKIECFDSVEISGVEDRFNASLGTYHDLLKIIK | |
| | DKDFLDNEENEDILEDIVLTLTLFEDREMIEERLKTYAHLFDD | |
| | KVMKQLKRRRYTGWGRLSRKLINGIRDKQSGKTILDFLKSDG | |
| | FANRAFAALIADDSLTFKEDIQKAQVSGQGDSLHEHIANLAGS | |
| | PAIKKGILQTVKVVDELVKVMGRHKPENIVIEMARENQTTQK | |
| | GQKNSRERMKRIEEGIKELGSQILKEHPVENTQLQNEKLYLYY | |
| | LQNGRDMYVDQELDINRLSDYDVDHIVPQSFLKDDSIDNKVL | |
| | TRSDKNRGKSDNVPSEEVVKKMKNYWRQLLNAKLITQRKFD | |
| | NLTKAERGGLSELDKAGFIKRQLVETRQITKHVAQILDSRMNT | |
| | KYDENDKLIREVKVITLKSKLVSDFRKDFQFYKVREINNYHH | |
| | AHDAYLNAVVGTALIKKYPKLESEFVYGDYKVYDVRKMIAK | |
| | SEQEIGKATAKYFFYSNIMNFFKTEITLANGEIRKRPLIETNGET | |
| | GEIVWDKGRDFATVRKVLSMPQVNIVKKTEVQTGGFSKESIL | |
| | PKRNSDKLIARKKDWDPKKYGGFDSPTVAYSVLVVAKVEKG | |
| | KSKKLKSVKELLGITIMERSSFEKNPIDFLEAKGYKEVKKDLII | |
| | KLPKYSLFELENGRKRMLASAGELQKGNELALPSKYVNFLYL | |
| | ASHYEKLKGSPEDNEQKQLFVEQHKHYLDEIIEQISEFSKRVIL | |
| | ADANLDKVLSAYNKHRDKPIREQAENIIHLFTLTNLGAPAAFK | |
| | YFDTTIDRKRYTSTKEVLDATLIHQSITGLYETRIDLSQLGGD | |
| HiFiCas9 | MDKKYSIGLDIGTNSVGWAVITDEYKVPSKKFKVLGNTDRHS | SEQ ID |
| | IKKNLIGALLFDSGETAEATRLKRTARRRYTRRKNRICYLQEIF | NO. |
| | SNEMAKVDDSFFHRLEESFLVEEDKKHERHPIFGNIVDEVAYH | 328 |
| | EKYPTIYHLRKKLVDSTDKADLRLIYLALAHMIKFRGHFLIEG | |
| | DLNPDNSDVDKLFIQLVQTYNQLFEENPINASGVDAKAILSAR | |
| | LSKSRRLENLIAQLPGEKKNGLFGNLIALSLGLTPNFKSNFDLA | |
| | EDAKLQLSKDTYDDDLDNLLAQIGDQYADLFLAAKNLSDAIL | |
| | LSDILRVNTEITKAPLSASMIKRYDEHHQDLTLLKALVRQQLP | |
| | EKYKEIFFDQSKNGYAGYIDGGASQEEFYKFIKPILEKMDGTE | |
| | ELLVKLNREDLLRKQRTFDNGSIPHQIHLGELHAILRRQEDFY | |
| | PFLKDNREKIEKILTFRIPYYVGPLARGNSRFAWMTRKSEETIT | |
| | PWNFEEVVDKGASAQSFIERMTNFDKNLPNEKVLPKHSLLYE | |
| | YFTVYNELTKVKYVTEGMRKPAFLSGEQKKAIVDLLFKTNRK | |
| | VTVKQLKEDYFKKIECFDSVEISGVEDRFNASLGTYHDLLKIIK | |
| | DKDFLDNEENEDILEDIVLTLTLFEDREMIEERLKTYAHLFDD | |
| | KVMKQLKRRRYTGWGRLSRKLINGIRDKQSGKTILDFLKSDG | |
| | FANANFMQLIHDDSLTFKEDIQKAQVSGQGDSLHEHIANLAG | |
| | SPAIKKGILQTVKVVDELVKVMGRHKPENIVIEMARENQTTQ | |
| | KGQKNSRERMKRIEEGIKELGSQILKEHPVENTQLQNEKLYLY | |
| | YLQNGRDMYVDQELDINRLSDYDVDHIVPQSFLKDDSIDNKV | |
| | LTRSDKNRGKSDNVPSEEVVKKMKNYWRQLLNAKLITQRKF | |
| | DNLTKAERGGLSELDKAGFIKRQLVETRQITKHVAQILDSRM | |
| | NTKYDENDKLIREVKVITLKSKLVSDFRKDFQFYKVREINNYH | |
| | HAHDAYLNAVVGTALIKKYPKLESEFVYGDYKVYDVRKMIA | |
| | KSEQEIGKATAKYFFYSNIMNFFKTEITLANGEIRKRPLIETNG | |
| | ETGEIVWDKGRDFATVRKVLSMPQVNIVKKTEVQTGGFSKES | |
| | ILPKRNSDKLIARKKDWDPKKYGGFDSPTVAYSVLVVAKVEK | |
| | GKSKKLKSVKELLGITIMERSSFEKNPIDFLEAKGYKEVKKDLI | |
| | IKLPKYSLFELENGRKRMLASAGELQKGNELALPSKYVNFLY | |
| | LASHYEKLKGSPEDNEQKQLFVEQHKHYLDEIIEQISEFSKRVI | |
| | LADANLDKVLSAYNKHRDKPIREQAENIIHLFTLTNLGAPAAF | |
| | KYFDTTIDRKRYTSTKEVLDATLIHQSITGLYETRIDLSQLGGD | |
| sniper- | MDKKYSIGLDIGTNSVGWAVITDEYKVPSKKFKVLGNTDRHS | SEQ ID |
| Cas9 | IKKNLIGALLFDSGETAEATRLKRTARRRYTRRKNRICYLQEIF | NO. |
| | SNEMAKVDDSFFHRLEESFLVEEDKKHERHPIFGNIVDEVAYH | 329 |
| | EKYPTIYHLRKKLVDSTDKADLRLIYLALAHMIKFRGHFLIEG | |
| | DLNPDNSDVDKLFIQLVQTYNQLFEENPINASGVDAKAILSAR | |
| | LSKSRRLENLIAQLPGEKKNGLFGNLIALSLGLTPNFKSNFDLA | |
| | EDAKLQLSKDTYDDDLDNLLAQIGDQYADLFLAAKNLSDAIL | |
| | LSDILRVNTEITKAPLSASMIKRYDEHHQDLTLLKALVRQQLP | |
| | EKYKEIFFDQSKNGYAGYIDGGASQEEFYKFIKPILEKMDGTE | |
| | ELLVKLNREDLLRKQRTFDNGSIPHQIHLGELHAILRRQEDFY | |
| | PFLKDNREKIEKILTFRIPYYVGPLARGNSRFAWMTRKSEETIT | |
| | PWNFEEVVDKGASAQSFIERMTNFDKNLPNEKVLPKHSLLYE | |
| | YFTVYNELTKVKYVTEGMRKPASLSGEQKKAIVDLLFKTNRK | |
| | VTVKQLKEDYFKKIECFDSVEISGVEDRFNASLGTYHDLLKIIK | |
| | DKDFLDNEENEDILEDIVLTLTLFEDREMIEERLKTYAHLFDD | |
| | KVMKQLKRRRYTGWGRLSRKLINGIRDKQSGKTILDFLKSDG | |
| | FANRNFMQLIHDDSLTFKEDIQKAQVSGQGDSLHEHIANLAG | |
| | SPAIKKGILQTVKVVDELVKVMGRHKPENIVIEIARENQTTQK | |
| | GQKNSRERMKRIEEGIKELGSQILKEHPVENTQLQNEKLYLYY | |
| | LQNGRDMYVDQELDINRLSDYDVDHIVPQSFLKDDSIDNKVL | |
| | TRSDKNRGKSDNVPSEEVVKKMKNYWRQLLNANLITQRKFD | |
| | NLTKAERGGLSELDKAGFIKRQLVETRQITKHVAQILDSRMNT | |
| | KYDENDKLIREVKVITLKSKLVSDFRKDFQFYKVREINNYHH | |
| | AHDAYLNAVVGTALIKKYPKLESEFVYGDYKVYDVRKMIAK | |
| | SEQEIGKATAKYFFYSNIMNFFKTEITLANGEIRKRPLIETNGET | |
| | GEIVWDKGRDFATVRKVLSMPQVNIVKKTEVQTGGFSKESIL | |
| | PKRNSDKLIARKKDWDPKKYGGFDSPTVAYSVLVVAKVEKG | |
| | KSKKLKSVKELLGITIMERSSFEKNPIDFLEAKGYKEVKKDLII | |
| | KLPKYSLFELENGRKRMLASAGELQKGNELALPSKYVNFLYL | |
| | ASHYEKLKGSPEDNEQKQLFVEQHKHYLDEIIEQISEFSKRVIL | |
| | ADANLDKVLSAYNKHRDKPIREQAENIIHLFTLTNLGAPAAFK | |
| | YFDTTIDRKRYTSTKEVLDATLIHQSITGLYETRIDLSQLGGD | |
| spG | MDKKYSIGLDIGTNSVGWAVITDEYKVPSKKFKVLGNTDRHS | SEQ ID |
| | IKKNLIGALLFDSGETAEATRLKRTARRRYTRRKNRICYLQEIF | NO. |
| | SNEMAKVDDSFFHRLEESFLVEEDKKHERHPIFGNIVDEVAYH | 330 |
| | EKYPTIYHLRKKLVDSTDKADLRLIYLALAHMIKFRGHFLIEG | |
| | DLNPDNSDVDKLFIQLVQTYNQLFEENPINASGVDAKAILSAR | |
| | LSKSRRLENLIAQLPGEKKNGLFGNLIALSLGLTPNFKSNFDLA | |
| | EDAKLQLSKDTYDDDLDNLLAQIGDQYADLFLAAKNLSDAIL | |
| | LSDILRVNTEITKAPLSASMIKRYDEHHQDLTLLKALVRQQLP | |
| | EKYKEIFFDQSKNGYAGYIDGGASQEEFYKFIKPILEKMDGTE | |
| | ELLVKLNREDLLRKQRTFDNGSIPHQIHLGELHAILRRQEDFY | |
| | PFLKDNREKIEKILTFRIPYYVGPLARGNSRFAWMTRKSEETIT | |
| | PWNFEEVVDKGASAQSFIERMTNFDKNLPNEKVLPKHSLLYE | |
| | YFTVYNELTKVKYVTEGMRKPAFLSGEQKKAIVDLLFKTNRK | |
| | VTVKQLKEDYFKKIECFDSVEISGVEDRFNASLGTYHDLLKIIK | |
| | DKDFLDNEENEDILEDIVLTLTLFEDREMIEERLKTYAHLFDD | |
| | KVMKQLKRRRYTGWGRLSRKLINGIRDKQSGKTILDFLKSDG | |
| | FANRNFMQLIHDDSLTFKEDIQKAQVSGQGDSLHEHIANLAG | |
| | SPAIKKGILQTVKVVDELVKVMGRHKPENIVIEMARENQTTQ | |
| | KGQKNSRERMKRIEEGIKELGSQILKEHPVENTQLQNEKLYLY | |
| | YLQNGRDMYVDQELDINRLSDYDVDHIVPQSFLKDDSIDNKV | |
| | LTRSDKNRGKSDNVPSEEVVKKMKNYWRQLLNAKLITQRKF | |
| | DNLTKAERGGLSELDKAGFIKRQLVETRQITKHVAQILDSRM | |
| | NTKYDENDKLIREVKVITLKSKLVSDFRKDFQFYKVREINNYH | |
| | HAHDAYLNAVVGTALIKKYPKLESEFVYGDYKVYDVRKMIA | |
| | KSEQEIGKATAKYFFYSNIMNFFKTEITLANGEIRKRPLIETNG | |
| | ETGEIVWDKGRDFATVRKVLSMPQVNIVKKTEVQTGGFSKES | |
| | ILPKRNSDKLIARKKDWDPKKYGGFLWPTVAYSVLVVAKVE | |
| | KGKSKKLKSVKELLGITIMERSSFEKNPIDFLEAKGYKEVKKD | |
| | LIIKLPKYSLFELENGRKRMLASAKQLQKGNELALPSKYVNFL | |
| | YLASHYEKLKGSPEDNEQKQLFVEQHKHYLDEIIEQISEFSKR | |
| | VILADANLDKVLSAYNKHRDKPIREQAENIIHLFTLTNLGAPA | |
| | AFKYFDTTIDRKQYRSTKEVLDATLIHQSITGLYETRIDLSQLG | |
| | GD | |
| SpRY | MDKKYSIGLDIGTNSVGWAVITDEYKVPSKKFKVLGNTDRHS | SEQ ID |
| | IKKNLIGALLFDSGETAERTRLKRTARRRYTRRKNRICYLQEIF | NO. |
| | SNEMAKVDDSFFHRLEESFLVEEDKKHERHPIFGNIVDEVAYH | 331 |
| | EKYPTIYHLRKKLVDSTDKADLRLIYLALAHMIKFRGHFLIEG | |
| | DLNPDNSDVDKLFIQLVQTYNQLFEENPINASGVDAKAILSAR | |
| | LSKSRRLENLIAQLPGEKKNGLFGNLIALSLGLTPNFKSNFDLA | |
| | EDAKLQLSKDTYDDDLDNLLAQIGDQYADLFLAAKNLSDAIL | |
| | LSDILRVNTEITKAPLSASMIKRYDEHHQDLTLLKALVRQQLP | |
| | EKYKEIFFDQSKNGYAGYIDGGASQEEFYKFIKPILEKMDGTE | |
| | ELLVKLNREDLLRKQRTFDNGSIPHQIHLGELHAILRRQEDFY | |
| | PFLKDNREKIEKILTFRIPYYVGPLARGNSRFAWMTRKSEETIT | |
| | PWNFEEVVDKGASAQSFIERMTNFDKNLPNEKVLPKHSLLYE | |
| | YFTVYNELTKVKYVTEGMRKPAFLSGEQKKAIVDLLFKTNRK | |
| | VTVKQLKEDYFKKIECFDSVEISGVEDRFNASLGTYHDLLKIIK | |
| | DKDFLDNEENEDILEDIVLTLTLFEDREMIEERLKTYAHLFDD | |
| | KVMKQLKRRRYTGWGRLSRKLINGIRDKQSGKTILDFLKSDG | |
| | FANRNFMQLIHDDSLTFKEDIQKAQVSGQGDSLHEHIANLAG | |
| | SPAIKKGILQTVKVVDELVKVMGRHKPENIVIEMARENQTTQ | |
| | KGQKNSRERMKRIEEGIKELGSQILKEHPVENTQLQNEKLYLY | |
| | YLQNGRDMYVDQELDINRLSDYDVDHIVPQSFLKDDSIDNKV | |
| | LTRSDKNRGKSDNVPSEEVVKKMKNYWRQLLNAKLITQRKF | |
| | DNLTKAERGGLSELDKAGFIKRQLVETRQITKHVAQILDSRM | |
| | NTKYDENDKLIREVKVITLKSKLVSDFRKDFQFYKVREINNYH | |
| | HAHDAYLNAVVGTALIKKYPKLESEFVYGDYKVYDVRKMIA | |
| | KSEQEIGKATAKYFFYSNIMNFFKTEITLANGEIRKRPLIETNG | |
| | ETGEIVWDKGRDFATVRKVLSMPQVNIVKKTEVQTGGFSKES | |
| | IRPKRNSDKLIARKKDWDPKKYGGFLWPTVAYSVLVVAKVE | |
| | KGKSKKLKSVKELLGITIMERSSFEKNPIDFLEAKGYKEVKKD | |
| | LIIKLPKYSLFELENGRKRMLASAKQLQKGNELALPSKYVNFL | |
| | YLASHYEKLKGSPEDNEQKQLFVEQHKHYLDEIIEQISEFSKR | |
| | VILADANLDKVLSAYNKHRDKPIREQAENIIHLFTLTRLGAPR | |
| | AFKYFDTTIDPKQYRSTKEVLDATLIHQSITGLYETRIDLSQLG | |
| | GD | |
| KKH | MGKRNYILGLDIGITSVGYGIIDYETRDVIDAGVRLFKEANVE | SEQ ID |
| SaCas9 | NNEGRRSKRGARRLKRRRRHRIQRVKKLLFDYNLLTDHSELS | NO. |
| | GINPYEARVKGLSQKLSEEEFSAALLHLAKRRGVHNVNEVEE | 332 |
| | DTGNELSTKEQISRNSKALEEKYVAELQLERLKKDGEVRGSIN | |
| | RFKTSDYVKEAKQLLKVQKAYHQLDQSFIDTYIDLLETRRTY | |
| | YEGPGEGSPFGWKDIKEWYEMLMGHCTYFPEELRSVKYAYN | |
| | ADLYNALNDLNNLVITRDENEKLEYYEKFQIIENVFKQKKKPT | |
| | LKQIAKEILVNEEDIKGYRVTSTGKPEFTNLKVYHDIKDITARK | |
| | EIIENAELLDQIAKILTIYQSSEDIQEELTNLNSELTQEEIEQISNL | |
| | KGYTGTHNLSLKAINLILDELWHTNDNQIAIFNRLKLVPKKVD | |
| | LSQQKEIPTTLVDDFILSPVVKRSFIQSIKVINAIIKKYGLPNDIII | |
| | ELAREKNSKDAQKMINEMQKRNRQTNERIEEIIRTTGKENAK | |
| | YLIEKIKLHDMQEGKCLYSLEAIPLEDLLNNPFNYEVDHIIPRS | |
| | VSFDNSFNNKVLVKQEENSKKGNRTPFQYLSSSDSKISYETFK | |
| | KHILNLAKGKGRISKTKKEYLLEERDINRFSVQKDFINRNLVD | |
| | TRYATRGLMNLLRSYFRVNNLDVKVKSINGGFTSFLRRKWKF | |
| | KKERNKGYKHHAEDALIIANADFIFKEWKKLDKAKKVMENQ | |
| | MFEEKQAESMPEIETEQEYKEIFITPHQIKHIKDFKDYKYSHRV | |
| | DKKPNRKLINDTLYSTRKDDKGNTLIVNNLNGLYDKDNDKL | |
| | KKLINKSPEKLLMYHHDPQTYQKLKLIMEQYGDEKNPLYKY | |
| | YEETGNYLTKYSKKDNGPVIKKIKYYGNKLNAHLDITDDYPN | |
| | SRNKVVKLSLKPYRFDVYLDNGVYKFVTVKNLDVIKKENYY | |
| | EVNSKCYEEAKKLKKISNQAEFIASFYKNDLIKINGELYRVIGV | |
| | NNDLLNRIEVNMIDITYREYLENMNDKRPPHIIKTIASKTQSIK | |
| | KYSTDILGNLYEVKSKKHPQIIKKG | |
| CjCas9 | MARILAFDIGISSIGWAFSENDELKDCGVRIFTKVENPKTGESL | SEQ ID |
| | ALPRRLARSARKRLARRKARLNHLKHLIANEFKLNYEDYQSF | NO. |
| | DESLAKAYKGSLISPYELRFRALNELLSKQDFARVILHIAKRR | 333 |
| | GYDDIKNSDDKEKGAILKAIKQNEEKLANYQSVGEYLYKEYF | |
| | QKFKENSKEFTNVRNKKESYERCIAQSFLKDELKLIFKKQREF | |
| | GFSFSKKFEEEVLSVAFYKRALKDFSHLVGNCSFFTDEKRAPK | |
| | NSPLAFMFVALTRIINLLNNLKNTEGILYTKDDLNALLNEVLK | |
| | NGTLTYKQTKKLLGLSDDYEFKGEKGTYFIEFKKYKEFIKALG | |
| | EHNLSQDDLNEIAKDITLIKDEIKLKKALAKYDLNQNQIDSLS | |
| | KLEFKDHLNISFKALKLVTPLMLEGKKYDEACNELNLKVAIN | |
| | EDKKDFLPAFNETYYKDEVTNPVVLRAIKEYRKVLNALLKKY | |
| | GKVHKINIELAREVGKNHSQRAKIEKEQNENYKAKKDAELEC | |
| | EKLGLKINSKNILKLRLFKEQKEFCAYSGEKIKISDLQDEKMLE | |
| | IDHIYPYSRSFDDSYMNKVLVFTKQNQEKLNQTPFEAFGNDS | |
| | AKWQKIEVLAKNLPTKKQKRILDKNYKDKEQKNFKDRNLND | |
| | TRYIARLVLNYTKDYLDFLPLSDDENTKLNDTQKGSKVHVEA | |
| | KSGMLTSALRHTWGFSAKDRNNHLHHAIDAVIIAYANNSIVK | |
| | AFSDFKKEQESNSAELYAKKISELDYKNKRKFFEPFSGFRQKV | |
| | LDKIDEIFVSKPERKKPSGALHEETFRKEEEFYQSYGGKEGVL | |
| | KALELGKIRKVNGKIVKNGDMFRVDIFKHKKTNKFYAVPIYT | |
| | MDFALKVLPNKAVARSKKGEIKDWILMDENYEFCFSLYKDSL | |
| | ILIQTKDMQEPEFVYYNAFTSSTVSLIVSKHDNKFETLSKNQKI | |
| | LFKNANEKEVIAKSIGIQNLKVFEKYIVSALGEVTKAEFRQRE | |
| | DFKK | |
| Cas9- | MDKKYSIGLDIGTNSVGWAVITDEYKVPSKKFKVLGNTDRHS | SEQ ID |
| NRRH | IKKNLIGALLFDSGETAEATRLKRTARRRYTRRKNRICYLQEIF | NO. |
| | SNEMAKVDDSFFHRLEESFLVEEDKKHERHPIFGNIVDEVAYH | 334 |
| | EKYPTIYHLRKKLVDSTDKADLRLIYLALAHMIKFRGHFLIEG | |
| | DLNPDNSDVDKLFIQLVQTYNQLFEENPINASGVDAKAILSAR | |
| | LSKSRRLENLIAQLPGEKKNGLFGNLIALSLGLTPNFKSNFDLA | |
| | EDAKLQLSKDTYDDDLDNLLAQIGDQYADLFLAAKNLSDAIL | |
| | LSDILRVNTEITKAPLSASMVKRYDEHHQDLTLLKALVRQQLP | |
| | EKYKEIFFDQSKNGYAGYIDGGASQEEFYKFIKPILEKMDGTE | |
| | ELLVKLNREDLLRKQRTFDNGIIPHQIHLGELHAILRRQGDFYP | |
| | FLKDNREKIEKILTFRIPYYVGPLARGNSRFAWMTRKSEETITP | |
| | WNFEEVVDKGASAQSFIERMTNFDKNLPNEKVLPKHSLLYEY | |
| | FTVYNELTKVKYVTEGMRKPAFLSGEQKKAIVDLLFKTNRKV | |
| | TVKQLKEDYFKKIECFDSVEISGVEDRFNASLGTYHDLLKIIKD | |
| | KDFLDNEENEDILEDIVLTLTLFEDREMIEERLKTYAHLFDDK | |
| | VMKQLKRLRYTGWGRLSRKLINGIRDKQSGKTILDFLKSDGF | |
| | ANRNFMQLIHDDSLTFKEDIQKAQVSGQGDSLHEHIANLAGS | |
| | PAIKKGILQTVKVVDELVKVMGGHKPENIVIEMARENQTTQK | |
| | GQKNSRERMKRIEEGIKELGSQILKEHPVENTQLQNEKLYLYY | |
| | LQNGRDMYVDQELDINRLSDYDVDHIVPQSFLKDDSIDNKVL | |
| | TRSDKNRGKSDNVPSEEVVKKMKNYWRQLLNAKLITQRKFD | |
| | NLTKAERGGLSELDKAGFIKRQLVETRQITKHVAQILDSRMNT | |
| | KYDENDKLIREVKVITLKSKLVSDFRKDFQFYKVREINNYHH | |
| | AHDAYLNAVVGTALIKKYPKLESEFVYGDYKVYDVRKMIAK | |
| | SEQEIGKATAKYFFYSNIMNFFKTEITLANGEIRKRPLIETNGET | |
| | GEIVWDKGRDFATVRKVLSMPQVNIVKKTEVQTGGFSKESIL | |
| | PKGNSDKLIARKKDWDPKKYGGFNSPTAAYSVLVVAKVEKG | |
| | KSKKLKSVKELLGITIMERSSFEKNPIGFLEAKGYKEVKKDLII | |
| | KLPKYSLFELENGRKRMLASAGVLHKGNELALPSKYVNFLYL | |
| | ASHYEKLKGSPEDNEQKQLFVEQHKHYLDEIIEQISEFSKRVIL | |
| | ADANLDKVLSAYNKHRDKPIREQAENIIHLFTLTNLGVPAAFK | |
| | YFDTTIDKKRYTSTKEVLDATLIHQSITGLYETRIDLSQLGGD | |
| Cas9- | MDKKYSIGLDIGTNSVGWAVITDEYKVPSKKFKVLGNTDRHS | SEQ ID |
| NRCH | IKKNLIGALLFDSGETAEATRLKRTARRRYTRRKNRICYLQEIF | NO. |
| | SNEMAKVDDSFFHRLEESFLVEEDKKHERHPIFGNIVDEVAYH | 335 |
| | EKYPTIYHLRKKLVDSTDKADLRLIYLALAHMIKFRGHFLIEG | |
| | DLNPDNSDVDKLFIQLVQTYNQLFEENPINASGVDAKAILSAR | |
| | LSKSRRLENLIAQLPGEKKNGLFGNLIALSLGLTPNFKSNFDLA | |
| | EDAKLQLSKDTYDDDLDNLLAQIGDQYADLFLAAKNLSDAIL | |
| | LSDILRVNTEITKAPLSASMVKRYDEHHQDLTLLKALVRQQLP | |
| | EKYKEIFFDQSKNGYAGYIDGGASQEEFYKFIKPILEKMDGTE | |
| | ELLVKLNREDLLRKQRTFDNGIIPHQIHLGELHAILRRQGDFYP | |
| | FLKDNREKIEKILTFRIPYYVGPLARGNSRFAWMTRKSEETITP | |
| | WNFEEVVDKGASAQSFIERMTNFDKNLPNEKVLPKHSLLYEY | |
| | FTVYNELTKVKYVTEGMRKPAFLSGEQKKAIVDLLFKTNRKV | |
| | TVKQLKEDYFKKIECFDSVEISGVEDRFNASLGTYHDLLKIIKD | |
| | KDFLDNEENEDILEDIVLTLTLFEDREMIEERLKTYAHLFDDK | |
| | VMKQLKRLRYTGWGRLSRKLINGIRDKQSGKTILDFLKSDGF | |
| | ANRNFMQLIHDDSLTFKEDIQKAQVSGQGDSLHEHIANLAGS | |
| | PAIKKGILQTVKVVDELVKVMGGHKPENIVIEMARENQTTQK | |
| | GQKNSRERMKRIEEGIKELGSQILKEHPVENTQLQNEKLYLYY | |
| | LQNGRDMYVDQELDINRLSDYDVDHIVPQSFLKDDSIDNKVL | |
| | TRSDKNRGKSDNVPSEEVVKKMKNYWRQLLNAKLITQRKFD | |
| | NLTKAERGGLSELDKAGFIKRQLVETRQITKHVAQILDSRMNT | |
| | KYDENDKLIREVKVITLKSKLVSDFRKDFQFYKVREINNYHH | |
| | AHDAYLNAVVGTALIKKYPKLESEFVYGDYKVYDVRKMIAK | |
| | SEQEIGKATAKYFFYSNIMNFFKTEITLANGEIRKRPLIETNGET | |
| | GEIVWDKGRDFATVRKVLSMPQVNIVKKTEVQTGGFSKESIL | |
| | PKGNSDKLIARKKDWDPKKYGGFNSPTVAYSVLVVAKVEKG | |
| | KSKKLKSVKELLGITIMERSSFEKNPIDFLEAKGYKEVKKDLII | |
| | KLPKYSLFELENGRKRMLASAGVLQKGNELALPSKYVNFLYL | |
| | ASHYEKLKGSPEDNEQKQLFVEQHKHYLDEIIEQISEFSKRVIL | |
| | ADANLDKVLSAYNKHRDKPIREQAENIIHLFTLTNLGAPAAFK | |
| | YFDTTINRKQYNTTKEVLDATLIRQSITGLYETRIDLSQLGGD | |
| Cas9- | MDKKYSIGLDIGTNSVGWAVITDEYKVPSKKFKVLGNTDRHS | SEQ ID |
| NRTH | IKKNLIGALLFDSGETAEATRLKRTARRRYTRRKNRICYLQEIF | NO. |
| | SNEMAKVDDSFFHRLEESFLVEEDKKHERHPIFGNIVDEVAYH | 336 |
| | EKYPTIYHLRKKLVDSTDKADLRLIYLALAHMIKFRGHFLIEG | |
| | DLNPDNSDVDKLFIQLVQTYNQLFEENPINASGVDAKAILSAR | |
| | LSKSRRLENLIAQLPGEKKNGLFGNLIALSLGLTPNFKSNFDLA | |
| | EDAKLQLSKDTYDDDLDNLLAQIGDQYADLFLAAKNLSDAIL | |
| | LSDILRVNTEITKAPLSASMVKRYDEHHQDLTLLKALVRQQLP | |
| | EKYKEIFFDQSKNGYAGYIDGGASQEEFYKFIKPILEKMDGTE | |
| | ELLVKLNREDLLRKQRTFDNGIIPHQIHLGELHAILRRQGDFYP | |
| | FLKDNREKIEKILTFRIPYYVGPLARGNSRFAWMTRKSEETITP | |
| | WNFEEVVDKGASAQSFIERMTNFDKNLPNEKVLPKHSLLYEY | |
| | FTVYNELTKVKYVTEGMRKPAFLSGEQKKAIVDLLFKTNRKV | |
| | TVKQLKEDYFKKIECFDSVEISGVEDRFNASLGTYHDLLKIIKD | |
| | KDFLDNEENEDILEDIVLTLTLFEDREMIEERLKTYAHLFDDK | |
| | VMKQLKRLRYTGWGRLSRKLINGIRDKQSGKTILDFLKSDGF | |
| | ANRNFMQLIHDDSLTFKEDIQKAQVSGQGDSLHEHIANLAGS | |
| | PAIKKGILQTVKVVDELVKVMGGHKPENIVIEMARENQTTQK | |
| | GQKNSRERMKRIEEGIKELGSQILKEHPVENTQLQNEKLYLYY | |
| | LQNGRDMYVDQELDINRLSDYDVDHIVPQSFLKDDSIDNKVL | |
| | TRSDKNRGKSDNVPSEEVVKKMKNYWRQLLNAKLITQRKFD | |
| | NLTKAERGGLSELDKAGFIKRQLVETRQITKHVAQILDSRMNT | |
| | KYDENDKLIREVKVITLKSKLVSDFRKDFQFYKVREINNYHH | |
| | AHDAYLNAVVGTALIKKYPKLESEFVYGDYKVYDVRKMIAK | |
| | SEQEIGKATAKYFFYSNIMNFFKTEITLANGEIRKRPLIETNGET | |
| | GEIVWDKGRDFATVRKVLSMPQVNIVKKTEVQTGGFSKESIL | |
| | PKGNSDKLIARKKDWDPKKYGGFNSPTVAYSVLVVAKVEKG | |
| | KSKKLKSVKELLGITIMERSSFEKNPIGFLEAKGYKEVKKDLII | |
| | KLPKYSLFELENGRKRMLASASVLHKGNELALPSKYVNFLYL | |
| | ASHYEKLKGSSEDNKQKQLFVEQHKHYLDEIIEQISEFSKRVIL | |
| | ADANLDKVLSAYNKHRDKPIREQAENIIHLFTLTNLGASAAFK | |
| | YFDTTIGRKLYTSTKEVLDATLIHQSITGLYETRIDLSQLGGD | |
| ScCpf1 | MQTLFENFTNQYPVSKTLRFELIPQGKTKDFIEQKGLLKKDED | SEQ ID |
| | RAEKYKKVKNIIDEYHKDFIEKSLNGLKLDGLEKYKTLYLKQ | NO. |
| | EKDDKDKKAFDKEKENLRKQIANAFRNNEKFKTLFAKELIKN | 337 |
| | DLMSFACEEDKKNVKEFEAFTTYFTGFHQNRANMYVADEKR | |
| | TAIASRLIHENLPKFIDNIKIFEKMKKEAPELLSPFNQTLKDMK | |
| | DVIKGTTLEEIFSLDYFNKTLTQSGIDIYNSVIGGRTPEEGKTKI | |
| | KGLNEYINTDFNQKQTDKKKRQPKFKQLYKQILSDRQSLSFIA | |
| | EAFKNDTEILEAIEKFYVNELLHFSNEGKSTNVLDAIKNAVSN | |
| | LESFNLTKMYFRSGASLTDVSRKVFGEWSIINRALDNYYATT | |
| | YPIKPREKSEKYEERKEKWLKQDFNVSLIQTAIDEYDNETVKG | |
| | KNSGKVIADYFAKFCDDKETDLIQKVNEGYIAVKDLLNTPCP | |
| | ENEKLGSNKDQVKQIKAFMDSIMDIMHFVRPLSLKDTDKEKD | |
| | ETFYSLFTPLYDHLTQTIALYNKVRNYLTQKPYSTEKIKLNFE | |
| | NSTLLGGWDLNKETDNTAIILRKDNLYYLGIMDKRHNRIFRN | |
| | VPKADKKDFCYEKMVYKLLPGANKMLPKVFFSQSRIQEFTPS | |
| | AKLLENYANETHKKGDNFNLNHCHKLIDFFKDSINKHEDWK | |
| | NFDFRFSATSTYADLSGFYHEVEHQGYKISFQSVADSFIDDLV | |
| | NEGKLYLFQIYNKDFSPFSKGKPNLHTLYWKMLFDENNLKDV | |
| | VYKLNGEAEVFYRKKSIAEKNTTIHKANESIINKNPDNPKATS | |
| | TFNYDIVKDKRYTIDKFQFHIPITMNFKAEGIFNMNQRVNQFL | |
| | KANPDINIIGIDRGERHLLYYALINQKGKILKQDTLNVIANEKQ | |
| | KVDYHNLLDKKEGDRATARQEWGVIETIKELKEGYLSQVIHK | |
| | LTDLMIENNAIIVMEDLNFGFKRGRQKVEKQVYQKFEKMLID | |
| | KLNYLVDKNKKANELGGLLNAFQLANKFESFQKMGKQNGFI | |
| | FYVPAWNTSKTDPATGFIDFLKPRYENLNQAKDFFEKFDSIRL | |
| | NSKADYFEFAFDFKNFTEKADGGRTKWTVCTTNEDRYAWNR | |
| | ALNNNRGSQEKYDITAELKSLFDGKVDYKSGKDLKQQIASQE | |
| | SADFFKALMKNLSITLSLRHNNGEKGDNEQDYILSPVADSKG | |
| | RFFDSRKADDDMPKNADANGAYHIALKGLWCLEQISKTDDL | |
| | KKVKLAISNKEWLEFVQTLKG | |
| PcCpf1 | MDSLKDFTNLYPVSKTLRFELKPVGKTLENIEKAGILKEDEHR | SEQ ID |
| | AESYRRVKKIIDTYHKVFIDSSLENMAKMGIENEIKAMLQSFC | NO. |
| | ELYKKDHRTEGEDKALDKIRAVLRGLIVGAFTGVCGRRENTV | 338 |
| | QNEKYESLFKEKLIKEILPDFVLSTEAESLPFSVEEATRSLKEFD | |
| | SFTSYFAGFYENRKNIYSTKPQSTAIAYRLIHENLPKFIDNILVF | |
| | QKIKEPIAKELEHIRADFSAGGYIKKDERLEDIFSLNYYIHVLS | |
| | QAGIEKYNALIGKIVTEGDGEMKGLNEHINLYNQQRGREDRL | |
| | PLFRPLYKQILSDREQLSYLPESFEKDEELLRALKEFYDHIAEDI | |
| | LGRTQQLMTSISEYDLSRIYVRNDSQLTDISKKMLGDWNAIY | |
| | MARERAYDHEQAPKRITAKYERDRIKALKGEESISLANLNSCI | |
| | AFLDNVRDCRVDTYLSTLGQKEGPHGLSNLVENVFASYHEAE | |
| | QLLSFPYPEENNLIQDKDNVVLIKNLLDNISDLQRFLKPLWGM | |
| | GDEPDKDERFYGEYNYIRGALDQVIPLYNKVRNYLTRKPYST | |
| | RKVKLNFGNSQLLSGWDRNKEKDNSCVILRKGQNFYLAIMN | |
| | NRHKRSFENKMLPEYKEGEPYFEKMDYKFLPDPNKMLPKVF | |
| | LSKKGIEIYKPSPKLLEQYGHGTHKKGDTFSMDDLHELIDFFK | |
| | HSIEAHEDWKQFGFKFSDTATYENVSSFYREVEDQGYKLSFR | |
| | KVSESYVYSLIDQGKLYLFQIYNKDFSPCSKGTPNLHTLYWR | |
| | MLFDERNLADVIYKLDGKAEIFFREKSLKNDHPTHPAGKPIKK | |
| | KSRQKKGEESLFEYDLVKDRRYTMDKFQFHVPITMNFKCSAG | |
| | SKVNDMVNAHIREAKDMHVIGIDRGERNLLYICVIDSRGTILD | |
| | QISLNTINDIDYHDLLESRDKDRQQEHRNWQTIEGIKELKQGY | |
| | LSQAVHRIAELMVAYKAVVALEDLNMGFKRGRQKVESSVYQ | |
| | QFEKQLIDKLNYLVDKKKRPEDIGGLLRAYQFTAPFKSFKEM | |
| | GKQNGFLFYIPAWNTSNIDPTTGFVNLFHVQYENVDKAKSFF | |
| | QKFDSISYNPKKDWFEFAFDYKNFTKKAEGSRSMWILCTHGS | |
| | RIKNFRNSQKNGQWDSEEFALTEAFKSLFVRYEIDYTADLKT | |
| | AIVDEKQKDFFVDLLKLFKLTVQMRNSWKEKDLDYLISPVAG | |
| | ADGRFFDTREGNKSLPKDADANGAYNIALKGLWALRQIRQTS | |
| | EGGKLKLAISNKEWLQFVQERSYEKD | |
| BpCpf1 | MLLYENYTKRNQITKSLRLELRPQGKTLRNIKELNLLEQDKAI | SEQ ID |
| | YALLERLKPVIDEGIKDIARDTLKNCELSFEKLYEHFLSGDKK | NO. |
| | AYAKESERLKKEIVKTLIKNLPEGIGKISEINSAKYLNGVLYDF | 339 |
| | IDKTHKDSEEKQNILSDILETKGYLALFSKFLTSRITTLEQSMP | |
| | KRVIENFEIYAANIPKMQDALERGAVSFAIEYESICSVDYYNQI | |
| | LSQEDIDSYNRLISGIMDEDGAKEKGINQTISEKNIKIKSEHLEE | |
| | KPFRILKQLHKQILEEREKAFTIDHIDSDEEVVQVTKEAFEQTK | |
| | EQWENIKKINGFYAKDPGDITLFIVVGPNQTHVLSQLIYGEHD | |
| | RIRLLLEEYEKNTLEVLPRRTKSEKARYDKFVNAVPKKVAKE | |
| | SHTFDGLQKMTGDDRLFILYRDELARNYMRIKEAYGTFERDI | |
| | LKSRRGIKGNRDVQESLVSFYDELTKFRSALRIINSGNDEKAD | |
| | PIFYNTFDGIFEKANRTYKAENLCRNYVTKSPADDARIMASCL | |
| | GTPARLRTHWWNGEENFAINDVAMIRRGDEYYYFVLTPDVK | |
| | PVDLKTKDETDAQIFVQRKGAKSFLGLPKALFKCILEPYFESPE | |
| | HKNDKNCVIEEYVSKPLTIDRRAYDIFKNGTFKKTNIGIDGLTE | |
| | EKFKDDCRYLIDVYKEFIAVYTRYSCFNMSGLKRADEYNDIG | |
| | EFFSDVDTRLCTMEWIPVSFERINDMVDKKEGLLFLVRSMFL | |
| | YNRPRKPYERTFIQLFSDSNMEHTSMLLNSRAMIQYRAASLPR | |
| | RVTHKKGSILVALRDSNGEHIPMHIREAIYKMKNNFDISSEDFI | |
| | MAKAYLAEHDVAIKKANEDIIRNRRYTEDKFFLSLSYTKNADI | |
| | SARTLDYINDKVEEDTQDSRMAVIVTRNLKDLTYVAVVDEK | |
| | NNVLEEKSLNEIDGVNYRELLKERTKIKYHDKTRLWQYDVSS | |
| | KGLKEAYVELAVTQISKLATKYNAVVVVESMSSTFKDKFSFL | |
| | DEQIFKAFEARLCARMSDLSFNTIKEGEAGSISNPIQVSNNNGN | |
| | SYQDGVIYFLNNAYTRTLCPDTGFVDVFDKTRLITMQSKRQFF | |
| | AKMKDIRIDDGEMLFTFNLEEYPTKRLLDRKEWTVKIAGDGS | |
| | YFDKDKGEYVYVNDIVREQIIPALLEDKAVFDGNMAEKFLDK | |
| | TAISGKSVELIYKWFANALYGIITKKDGEKIYRSPITGTEIDVSK | |
| | NTTYNFGKKFMFKQEYRGDGDFLDAFLNYMQAQDIAV | |
| LiCpf1 | MEDYSGFVNIYSIQKTLRFELKPVGKTLEHIEKKGFLKKDKIRAEDY | SEQ ID |
| | KAVKKIIDKYHRAYIEEVFDSVLHQKKKKDKTRFSTQFIKEIKEFSEL | NO. 340 |
| | YYKTEKNIPDKERLEALSEKLRKMLVGAFKGEFSEEVAEKYKNLFS | |
| | KELIRNEIEKFCETDEERKQVSNFKSFTTYFTGFHSNRQNIYSDEKKS | |
| | TAIGYRIIHQNLPKFLDNLKIIESIQRRFKDFPWSDLKKNLKKIDKNIK | |
| | LTEYFSIDGFVNVLNQKGIDAYNTILGGKSEESGEKIQGLNEYINLYR | |
| | QKNNIDRKNLPNVKILFKQILGDRETKSFIPEAFPDDQSVLNSITEFA | |
| | KYLKLDKKKKSIIAELKKFLSSFNRYELDGIYLANDNSLASISTFLFD | |
| | DWSFIKKSVSFKYDESVGDPKKKIKSPLKYEKEKEKWLKQKYYTIS | |
| | FLNDAIESYSKSQDEKRVKIRLEAYFAEFKSKDDAKKQFDLLERIEE | |
| | AYAIVEPLLGAEYPRDRNLKADKKEVGKIKDFLDSIKSLQFFLKPLL | |
| | SAEIFDEKDLGFYNQLEGYYEEIDSIGHLYNKVRNYLTGKIYSKEKF | |
| | KLNFENSTLLKGWDENREVANLCVIFREDQKYYLGVMDKENNTILS | |
| | DIPKVKPNELFYEKMVYKLIPTPHMQLPRIIFSSDNLSIYNPSKSILKIR | |
| | EAKSFKEGKNFKLKDCHKFIDFYKESISKNEDWSRFDFKFSKTSSYE | |
| | NISEFYREVERQGYNLDFKKVSKFYIDSLVEDGKLYLFQIYNKDFSIF | |
| | SKGKPNLHTIYFRSLFSKENLKDVCLKLNGEAEMFFRKKSINYDEKK | |
| | KREGHHPELFEKLKYPILKDKRYSEDKFQFHLPISLNFKSKERLNFNL | |
| | KVNEFLKRNKDINIIGIDRGERNLLYLVMINQKGEILKQTLLDSMQS | |
| | GKGRPEINYKEKLQEKEIERDKARKSWGTVENIKELKEGYLSIVIHQI | |
| | SKLMVENNAIVVLEDLNIGFKRGRQKVERQVYQKFEKMLIDKLNFL | |
| | VFKENKPTEPGGVLKAYQLTDEFQSFEKLSKQTGFLFYVPSWNTSKI | |
| | DPRTGFIDFLHPAYENIEKAKQWINKFDSIRFNSKMDWFEFTADTRK | |
| | FSENLMLGKNRVWVICTTNVERYFTSKTANSSIQYNSIQITEKLKELF | |
| | VDIPFSNGQDLKPEILRKNDAVFFKSLLFYIKTTLSLRQNNGKKGEEE | |
| | KDFILSPVVDSKGRFFNSLEASDDEPKDADANGAYHIALKGLMNLL | |
| | VLNETKEENLSRPKWKIKNKDWLEFVWERNR | |
| PmCpf1 | MKTQHFFEDFTSLYSLSKTIRFELKPIGKTLENIKKNGLIRRDEQRLD | SEQ ID |
| | DYEKLKKVIDEYHEDFIANILSSFSFSEEILQSYIQNLSESEARAKIEK | NO. 341 |
| | TMRDTLAKAFSEDERYKSIFKKELVKKDIPVWCPAYKSLCKKFDNF | |
| | TTSLVPFHENRKNLYTSNEITASIPYRIVHVNLPKFIQNIEALCELQKK | |
| | MGADLYLEMMENLRNVWPSFVKTPDDLCNLKTYNHLMVQSSISEY | |
| | NRFVGGYSTEDGTKHQGINEWINIYRQRNKEMRLPGLVFLHKQILA | |
| | KVDSSSFISDTLENDDQVFCVLRQFRKLFWNTVSSKEDDAASLKDL | |
| | FCGLSGYDPEAIYVSDAHLATISKNIFDRWNYISDAIRRKTEVLMPR | |
| | KKESVERYAEKISKQIKKRQSYSLAELDDLLAHYSEESLPAGFSLLS | |
| | YFTSLGGQKYLVSDGEVILYEEGSNIWDEVLIAFRDLQVILDKDFTE | |
| | KKLGKDEEAVSVIKKALDSALRLRKFFDLLSGTGAEIRRDSSFYALY | |
| | TDRMDKLKGLLKMYDKVRNYLTKKPYSIEKFKLHFDNPSLLSGWD | |
| | KNKELNNLSVIFRQNGYYYLGIMTPKGKNLFKTLPKLGAEEMFYEK | |
| | MEYKQIAEPMLMLPKVFFPKKTKPAFAPDQSVVDIYNKKTFKTGQK | |
| | GFNKKDLYRLIDFYKEALTVHEWKLFNFSFSPTEQYRNIGEFFDEVR | |
| | EQAYKVSMVNVPASYIDEAVENGKLYLFQIYNKDFSPYSKGIPNLH | |
| | TLYWKALFSEQNQSRVYKLCGGGELFYRKASLHMQDTTVHPKGISI | |
| | HKKNLNKKGETSLFNYDLVKDKRFTEDKFFFHVPISINYKNKKITNV | |
| | NQMVRDYIAQNDDLQIIGIDRGERNLLYISRIDTRGNLLEQFSLNVIE | |
| | SDKGDLRTDYQKILGDREQERLRRRQEWKSIESIKDLKDGYMSQVV | |
| | HKICNMVVEHKAIVVLENLNLSFMKGRKKVEKSVYEKFERMLVDK | |
| | LNYLVVDKKNLSNEPGGLYAAYQLTNPLFSFEELHRYPQSGILFFVD | |
| | PWNTSLTDPSTGFVNLLGRINYTNVGDARKFFDRFNAIRYDGKGNIL | |
| | FDLDLSRFDVRVETQRKLWTLTTFGSRIAKSKKSGKWMVERIENLS | |
| | LCFLELFEQFNIGYRVEKDLKKAILSQDRKEFYVRLIYLFNLMMQIR | |
| | NSDGEEDYILSPALNEKNLQFDSRLIEAKDLPVDADANGAYNVARK | |
| | GLMVVQRIKRGDHESIHRIGRAQWLRYVQEGIVE | |
| Lb2Cpf1 | MYYESLTKQYPVSKTIRNELIPIGKTLDNIRQNNILESDVKRKQNYE | SEQ ID |
| | HVKGILDEYHKQLINEALDNCTLPSLKIAAEIYLKNQKEVSDREDFN | NO. 342 |
| | KTQDLLRKEVVEKLKAHENFTKIGKKDILDLLEKLPSISEDDYNALE | |
| | SFRNFYTYFTSYNKVRENLYSDKEKSSTVAYRLINENFPKFLDNVKS | |
| | YRFVKTAGILADGLGEEEQDSLFIVETENKTLTQDGIDTYNSQVGKI | |
| | NSSINLYNQKNQKANGFRKIPKMKMLYKQILSDREESFIDEFQSDEV | |
| | LIDNVESYGSVLIESLKSSKVSAFFDALRESKGKNVYVKNDLAKTA | |
| | MSNIVFENWRTFDDLLNQEYDLANENKKKDDKYFEKRQKELKKN | |
| | KSYSLEHLCNLSEDSCNLIENYIHQISDDIENIIINNETFLRIVINEHDR | |
| | SRKLAKNRKAVKAIKDFLDSIKVLERELKLINSSGQELEKDLIVYSA | |
| | HEELLVELKQVDSLYNMTRNYLTKKPFSTEKVKLNFNRSTLLNGW | |
| | DRNKETDNLGVLLLKDGKYYLGIMNTSANKAFVNPPVAKTEKVFK | |
| | KVDYKLLPVPNQMLPKVFFAKSNIDFYNPSSEIYSNYKKGTHKKGN | |
| | MFSLEDCHNLIDFFKESISKHEDWSKFGFKFSDTASYNDISEFYREVE | |
| | KQGYKLTYTDIDETYINDLIERNELYLFQIYNKDFSMYSKGKLNLHT | |
| | LYFMMLFDQRNIDDVVYKLNGEAEVFYRPASISEDELIIHKAGEEIK | |
| | NKNPNRARTKETSTFSYDIVKDKRYSKDKFTLHIPITMNFGVDEVKR | |
| | FNDAVNSAIRIDENVNVIGIDRGERNLLYVVVIDSKGNILEQISLNSII | |
| | NKEYDIETDYHALLDEREGGRDKARKDWNTVENIRDLKAGYLSQV | |
| | VNVVAKLVLKYNAIICLEDLNFGFKRGRQKVEKQVYQKFEKMLID | |
| | KLNYLVIDKSREQTSPKELGGALNALQLTSKFKSFKELGKQSGVIYY | |
| | VPAYLTSKIDPTTGFANLFYMKCENVEKSKRFFDGFDFIRFNALENV | |
| | FEFGFDYRSFTQRACGINSKWTVCTNGERIIKYRNPDKNNMFDEKV | |
| | VVVTDEMKNLFEQYKIPYEDGRNVKDMIISNEEAEFYRRLYRLLQQ | |
| | TLQMRNSTSDGTRDYIISPVKNKREAYFNSELSDGSVPKDADANGA | |
| | YNIARKGLWVLEQIRQKSEGEKINLAMTNAEWLEYAQTHLL | |
| PbCpf1 | MENIFDQFIGKYSLSKTLRFELKPVGKTEDFLKINKVFEKDQTIDDSY | SEQ ID |
| | NQAKFYFDSLHQKFIDAALASDKTSELSFQNFADVLEKQNKIILDKK | NO. 343 |
| | REMGALRKRDKNAVGIDRLQKEINDAEDIIQKEKEKIYKDVRTLFD | |
| | NEAESWKTYYQEREVDGKKITFSKADLKQKGADFLTAAGILKVLK | |
| | YEFPEEKEKEFQAKNQPSLFVEEKENPGQKRYIFDSFDKFAGYLTKF | |
| | QQTKKNLYAADGTSTAVATRIADNFIIFHQNTKVFRDKYKNNHTDL | |
| | GFDEENIFEIERYKNCLLQREIEHIKNENSYNKIIGRINKKIKEYRDQK | |
| | AKDTKLTKSDFPFFKNLDKQILGEVEKEKQLIEKTREKTEEDVLIERF | |
| | KEFIENNEERFTAAKKLMNAFCNGEFESEYEGIYLKNKAINTISRRW | |
| | FVSDRDFELKLPQQKSKNKSEKNEPKVKKFISIAEIKNAVEELDGDIF | |
| | KAVFYDKKIIAQGGSKLEQFLVIWKYEFEYLFRDIERENGEKLLGYD | |
| | SCLKIAKQLGIFPQEKEAREKATAVIKNYADAGLGIFQMMKYFSLD | |
| | DKDRKNTPGQLSTNFYAEYDGYYKDFEFIKYYNEFRNFITKKPFDE | |
| | DKIKLNFENGALLKGWDENKEYDFMGVILKKEGRLYLGIMHKNHR | |
| | KLFQSMGNAKGDNANRYQKMIYKQIADASKDVPRLLLTSKKAME | |
| | KFKPSQEILRIKKEKTFKRESKNFSLRDLHALIEYYRNCIPQYSNWSF | |
| | YDFQFQDTGKYQNIKEFTDDVQKYGYKISFRDIDDEYINQALNEGK | |
| | MYLFEVVNKDIYNTKNGSKNLHTLYFEHILSAENLNDPVFKLSGMA | |
| | EIFQRQPSVNEREKITTQKNQCILDKGDRAYKYRRYTEKKIMFHMSL | |
| | VLNTGKGEIKQVQFNKIINQRISSSDNEMRVNVIGIDRGEKNLLYYS | |
| | VVKQNGEIIEQASLNEINGVNYRDKLIEREKERLKNRQSWKPVVKIK | |
| | DLKKGYISHVIHKICQLIEKYSAIVVLEDLNMRFKQIRGGIERSVYQQ | |
| | FEKALIDKLGYLVFKDNRDLRAPGGVLNGYQLSAPFVSFEKMRKQT | |
| | GILFYTQAEYTSKTDPITGFRKNVYISNSASLDKIKEAVKKFDAIGW | |
| | DGKEQSYFFKYNPYNLADEKYKNSTVSKEWAIFASAPRIRRQKGED | |
| | GYWKYDRVKVNEEFEKLLKVWNFVNPKATDIKQEIIKKEKAGDLQ | |
| | GEKELDGRLRNFWHSFIYLFNLVLELRNSFSLQIKIKAGEVIAVDEG | |
| | VDFIASPVKPFFTTPNPYIPSNLCWLAVENADANGAYNIARKGVMIL | |
| | KKIREHAKKDPEFKKLPNLFISNAEWDEAARDWGKYAGTTALNLD | |
| | H | |
| PeCpf1 | MSNFFKNFTNLYELSKTLRFELKPVGDTLTNMKDHLEYDEKLQTFL | SEQ ID |
| | KDQNIDDAYQALKPQFDEIHEEFITDSLESKKAKEIDFSEYLDLFQEK | NO. 344 |
| | KELNDSEKKLRNKIGETFNKAGEKWKKEKYPQYEWKKGSKIANGA | |
| | DILSCQDMLQFIKYKNPEDEKIKNYIDDTLKGFFTYFGGFNQNRANY | |
| | YETKKEASTAVATRIVHENLPKFCDNVIQFKHIIKRKKDGTVEKTER | |
| | KTEYLNAYQYLKNNNKITQIKDAETEKMIESTPIAEKIFDVYYFSSCL | |
| | SQKQIEEYNRIIGHYNLLINLYNQAKRSEGKHLSANEKKYKDLPKFK | |
| | TLYKQIGCGKKKDLFYTIKCDTEEEANKSRNEGKESHSVEEIINKAQ | |
| | EAINKYFKSNNDCENINTVPDFINYILTKENYEGVYWSKAAMNTISD | |
| | KYFANYHDLQDRLKEAKVFQKADKKSEDDIKIPEAIELSGLFGVLDS | |
| | LADWQTTLFKSSILSNEDKLKIITDSQTPSEALLKMIFNDIEKNMESF | |
| | LKETNDIITLKKYKGNKEGTEKIKQWFDYTLAINRMLKYFLVKENKI | |
| | KGNSLDTNISEALKTLIYSDDAEWFKWYDALRNYLTQKPQDEAKE | |
| | NKLKLNFDNPSLAGGWDVNKECSNFCVILKDKNEKKYLAIMKKGE | |
| | NTLFQKEWTEGRGKNLTKKSNPLFEINNCEILSKMEYDFWADVSK | |
| | MIPKCSTQLKAVVNHFKQSDNEFIFPIGYKVTSGEKFREECKISKQDF | |
| | ELNNKVFNKNELSVTAMRYDLSSTQEKQYIKAFQKEYWELLFKQE | |
| | KRDTKLTNNEIFNEWINFCNKKYSELLSWERKYKDALTNWINFCKY | |
| | FLSKYPKTTLFNYSFKESENYNSLDEFYRDVDICSYKLNINTTINKSIL | |
| | DRLVEEGKLYLFEIKNQDSNDGKSIGHKNNLHTIYWNAIFENFDNRP | |
| | KLNGEAEIFYRKAISKDKLGIVKGKKTKNGTEIIKNYRFSKEKFILHV | |
| | PITLNFCSNNEYVNDIVNTKFYNFSNLHFLGIDRGEKHLAYYSLVNK | |
| | NGEIVDQGTLNLPFTDKDGNQRSIKKEKYFYNKQEDKWEAKEVDC | |
| | WNYNDLLDAMASNRDMARKNWQRIGTIKEAKNGYVSLVIRKIADL | |
| | AVNNERPAFIVLEDLNTGFKRSRQKIDKSVYQKFELALAKKLNFLV | |
| | DKNAKRDEIGSPTKALQLTPPVNNYGDIENKKQAGIMLYTRANYTS | |
| | QTDPATGWRKTIYLKAGPEETTYKKDGKIKNKSVKDQIIETFTDIGF | |
| | DGKDYYFEYDKGEFVDEKTGEIKPKKWRLYSGENGKSLDRFRGER | |
| | EKDKYEWKIDKIDIVKILDDLFVNFDKNISLLKQLKEGVELTRNNEH | |
| | GTGESLRFAINLIQQIRNTGNNERDNDFILSPVRDENGKHFDSREYW | |
| | DKETKGEKISMPSSGDANGAFNIARKGIIMNAHILANSDSKDLSLFV | |
| | SDEEWDLHLNNKTEWKKQLNIFSSRKAMAKRKK | |
| PdCpf1 | MENYQEFTNLFQLNKTLRFELKPIGKTCELLEEGKIFASGSFLEKDK | SEQ ID |
| | VRADNVSYVKKEIDKKHKIFIEETLSSFSISNDLLKQYFDCYNELKAF | NO. 345 |
| | KKDCKSDEEEVKKTALRNKCTSIQRAMREAISQAFLKSPQKKLLAIK | |
| | NLIENVFKADENVQHFSEFTSYFSGFETNRENFYSDEEKSTSIAYRLV | |
| | HDNLPIFIKNIYIFEKLKEQFDAKTLSEIFENYKLYVAGSSLDEVFSLE | |
| | YFNNTLTQKGIDNYNAVIGKIVKEDKQEIQGLNEHINLYNQKHKDR | |
| | RLPFFISLKKQILSDREALSWLPDMFKNDSEVIKALKGFYIEDGFENN | |
| | VLTPLATLLSSLDKYNLNGIFIRNNEALSSLSQNVYRNFSIDEAIDAN | |
| | AELQTFNNYELIANALRAKIKKETKQGRKSFEKYEEYIDKKVKAIDS | |
| | LSIQEINELVENYVSEFNSNSGNMPRKVEDYFSLMRKGDFGSNDLIE | |
| | NIKTKLSAAEKLLGTKYQETAKDIFKKDENSKLIKELLDATKQFQHF | |
| | IKPLLGTGEEADRDLVFYGDFLPLYEKFEELTLLYNKVRNRLTQKPY | |
| | SKDKIRLCFNKPKLMTGWVDSKTEKSDNGTQYGGYLFRKKNEIGE | |
| | YDYFLGISSKAQLFRKNEAVIGDYERLDYYQPKANTIYGSAYEGEN | |
| | SYKEDKKRLNKVIIAYIEQIKQTNIKKSIIESISKYPNISDDDKVTPSSL | |
| | LEKIKKVSIDSYNGILSFKSFQSVNKEVIDNLLKTISPLKNKAEFLDLI | |
| | NKDYQIFTEVQAVIDEICKQKTFIYFPISNVELEKEMGDKDKPLCLFQ | |
| | ISNKDLSFAKTFSANLRKKRGAENLHTMLFKALMEGNQDNLDLGS | |
| | GAIFYRAKSLDGNKPTHPANEAIKCRNVANKDKVSLFTYDIYKNRR | |
| | YMENKFLFHLSIVQNYKAANDSAQLNSSATEYIRKADDLHIIGIDRG | |
| | ERNLLYYSVIDMKGNIVEQDSLNIIRNNDLETDYHDLLDKREKERK | |
| | ANRQNWEAVEGIKDLKKGYLSQAVHQIAQLMLKYNAIIALEDLGQ | |
| | MFVTRGQKIEKAVYQQFEKSLVDKLSYLVDKKRPYNELGGILKAY | |
| | QLASSITKNNSDKQNGFLFYVPAWNTSKIDPVTGFTDLLRPKAMTIK | |
| | EAQDFFGAFDNISYNDKGYFEFETNYDKFKIRMKSAQTRWTICTFG | |
| | NRIKRKKDKNYWNYEEVELTEEFKKLFKDSNIDYENCNLKEEIQNK | |
| | DNRKFFDDLIKLLQLTLQMRNSDDKGNDYIISPVANAEGQFFDSRN | |
| | GDKKLPLDADANGAYNIARKGLWNIRQIKQTKNDKKLNLSISSTEW | |
| | LDFVREKPYLK | |
| MbCpf1 | MLFQDFTHLYPLSKTVRFELKPIDRTLEHIHAKNFLSQDETMADMH | SEQ ID |
| | QKVKVILDDYHRDFIADMMGEVKLTKLAEFYDVYLKFRKNPKDDE | NO. 346 |
| | LQKQLKDLQAVLRKEIVKPIGNGGKYKAGYDRLFGAKLFKDGKEL | |
| | GDLAKFVIAQEGESSPKLAHLAHFEKFSTYFTGFHDNRKNMYSDED | |
| | KHTAIAYRLIHENLPRFIDNLQILTTIKQKHSALYDQIINELTASGLDV | |
| | SLASHLDGYHKLLTQEGITAYNTLLGGISGEAGSPKIQGINELINSHH | |
| | NQHCHKSERIAKLRPLHKQILSDGMSVSFLPSKFADDSEMCQAVNE | |
| | FYRHYADVFAKVQSLFDGFDDHQKDGIYVEHKNLNELSKQAFGDF | |
| | ALLGRVLDGYYVDVVNPEFNERFAKAKTDNAKAKLTKEKDKFIKG | |
| | VHSLASLEQAIEHYTARHDDESVQAGKLGQYFKHGLAGVDNPIQKI | |
| | HNNHSTIKGFLERERPAGERALPKIKSGKNPEMTQLRQLKELLDNAL | |
| | NVAHFAKLLTTKTTLDNQDGNFYGEFGVLYDELAKIPTLYNKVRD | |
| | YLSQKPFSTEKYKLNFGNPTLLNGWDLNKEKDNFGVILQKDGCYY | |
| | LALLDKAHKKVFDNAPNTGKSIYQKMIYKYLEVRKQFPKVFFSKEA | |
| | IAINYHPSKELVEIKDKGRQRSDDERLKLYRFILECLKIHPKYDKKFE | |
| | GAIGDIQLFKKDKKGREVPISEKDLFDKINGIFSSKPKLEMEDFFIGEF | |
| | KRYNPSQDLVDQYNIYKKIDSNDNRKKENFYNNHPKFKKDLVRYY | |
| | YESMCKHEEWEESFEFSKKLQDIGCYVDVNELFTEIETRRLNYKISF | |
| | CNINADYIDELVEQGQLYLFQIYNKDFSPKAHGKPNLHTLYFKALFS | |
| | EDNLADPIYKLNGEAQIFYRKASLDMNETTIHRAGEVLENKNPDNP | |
| | KKRQFVYDIIKDKRYTQDKFMLHVPITMNFGVQGMTIKEFNKKVN | |
| | QSIQQYDEVNVIGIDRGERHLLYLTVINSKGEILEQCSLNDITTASAN | |
| | GTQMTTPYHKILDKREIERLNARVGWGEIETIKELKSGYLSHVVHQI | |
| | SQLMLKYNAIVVLEDLNFGFKRGRFKVEKQIYQNFENALIKKLNHL | |
| | VLKDKADDEIGSYKNALQLTNNFTDLKSIGKQTGFLFYVPAWNTSK | |
| | IDPETGFVDLLKPRYENIAQSQAFFGKFDKICYNADKDYFEFHIDYA | |
| | KFTDKAKNSRQIWTICSHGDKRYVYDKTANQNKGAAKGINVNDEL | |
| | KSLFARHHINEKQPNLVMDICQNNDKEFHKSLMYLLKTLLALRYSN | |
| | ASSDEDFILSPVANDEGVFFNSALADDTQPQNADANGAYHIALKGL | |
| | WLLNELKNSDDLNKVKLAIDNQTWLNFAQNR | |
| EeCpf1 | MNGNRSIVYREFVGVIPVAKTLRNELRPVGHTQEHIIQNGLIQEDEL | SEQ ID |
| | RQEKSTELKNIMDDYYREYIDKSLSGVTDLDFTLLFELMNLVQSSPS | NO. 347 |
| | KDNKKALEKEQSKMREQICTHLQSDSNYKNIFNAKLLKEILPDFIKN | |
| | YNQYDVKDKAGKLETLALFNGFSTYFTDFFEKRKNVFTKEAVSTSI | |
| | AYRIVHENSLIFLANMTSYKKISEKALDEIEVIEKNNQDKMGDWEL | |
| | NQIFNPDFYNMVLIQSGIDFYNEICGVVNAHMNLYCQQTKNNYNLF | |
| | KMRKLHKQILAYTSTSFEVPKMFEDDMSVYNAVNAFIDETEKGNII | |
| | GKLKDIVNKYDELDEKRIYISKDFYETLSCFMSGNWNLITGCVENFY | |
| | DENIHAKGKSKEEKVKKAVKEDKYKSINDVNDLVEKYIDEKERNEF | |
| | KNSNAKQYIREISNIITDTETAHLEYDDHISLIESEEKADEMKKRLDM | |
| | YMNMYHWAKAFIVDEVLDRDEMFYSDIDDIYNILENIVPLYNRVRN | |
| | YVTQKPYNSKKIKLNFQSPTLANGWSQSKEFDNNAIILIRDNKYYLA | |
| | IFNAKNKPDKKIIQGNSDKKNDNDYKKMVYNLLPGANKMLPKVFL | |
| | SKKGIETFKPSDYIISGYNAHKHIKTSENFDISFCRDLIDYFKNSIEKH | |
| | AEWRKYEFKFSATDSYSDISEFYREVEMQGYRIDWTYISEADINKLD | |
| | EEGKIYLFQIYNKDFAENSTGKENLHTMYFKNIFSEENLKDIIIKLNG | |
| | QAELFYRRASVKNPVKHKKDSVLVNKTYKNQLDNGDVVRIPIPDDI | |
| | YNEIYKMYNGYIKESDLSEAAKEYLDKVEVRTAQKDIVKDYRYTV | |
| | DKYFIHTPITINYKVTARNNVNDMVVKYIAQNDDIHVIGIDRGERNL | |
| | IYISVIDSHGNIVKQKSYNILNNYDYKKKLVEKEKTREYARKNWKSI | |
| | GNIKELKEGYISGVVHEIAMLIVEYNAIIAMEDLNYGFKRGRFKVER | |
| | QVYQKFESMLINKLNYFASKEKSVDEPGGLLKGYQLTYVPDNIKNL | |
| | GKQCGVIFYVPAAFTSKIDPSTGFISAFNFKSISTNASRKQFFMQFDEI | |
| | RYCAEKDMFSFGFDYNNFDTYNITMGKTQWTVYTNGERLQSEFNN | |
| | ARRTGKTKSINLTETIKLLLEDNEINYADGHDIRIDMEKMDEDKKSE | |
| | FFAQLLSLYKLTVQMRNSYTEAEEQENGISYDKIISPVINDEGEFFDS | |
| | DNYKESDDKECKMPKDADANGAYCIALKGLYEVLKIKSEWTEDGF | |
| | DRNCLKLPHAEWLDFIQNKRYE | |
| CmtCpf1 | MNNYDEFTKLYPIQKTIRFELKPQGRTMEHLETFNFFEEDRDRAEKY | SEQ ID |
| | KILKEAIDEYHKKFIDEHLTNMSLDWNSLKQISEKYYKSREEKDKK | NO. 348 |
| | VFLSEQKRMRQEIVSEFKKDDRFKDLFSKKLFSELLKEEIYKKGNHQ | |
| | EIDALKSFDKFSGYFIGLHENRKNMYSDGDEITAISNRIVNENFPKFL | |
| | DNLQKYQEARKKYPEWIIKAESALVAHNIKMDEVFSLEYFNKVLNQ | |
| | EGIQRYNLALGGYVTKSGEKMMGLNDALNLAHQSEKSSKGRIHMT | |
| | PLFKQILSEKESFSYIPDVFTEDSQLLPSIGGFFAQIENDKDGNIFDRA | |
| | LELISSYAEYDTERIYIRQADINRVSNVIFGEWGTLGGLMREYKADSI | |
| | NDINLERTCKKVDKWLDSKEFALSDVLEAIKRTGNNDAFNEYISKM | |
| | RTAREKIDAARKEMKFISEKISGDEESIHIIKTLLDSVQQFLHFFNLFK | |
| | ARQDIPLDGAFYAEFDEVHSKLFAIVPLYNKVRNYLTKNNLNTKKI | |
| | KLNFKNPTLANGWDQNKVYDYASLIFLRDGNYYLGIINPKRKKNIK | |
| | FEQGSGNGPFYRKMVYKQIPGPNKNLPRVFLTSTKGKKEYKPSKEII | |
| | EGYEADKHIRGDKFDLDFCHKLIDFFKESIEKHKDWSKFNFYFSPTE | |
| | SYGDISEFYLDVEKQGYRMHFENISAETIDEYVEKGDLFLFQIYNKD | |
| | FVKAATGKKDMHTIYWNAAFSPENLQDVVVKLNGEAELFYRDKSD | |
| | IKEIVHREGEILVNRTYNGRTPVPDKIHKKLTDYHNGRTKDLGEAKE | |
| | YLDKVRYFKAHYDITKDRRYLNDKIYFHVPLTLNFKANGKKNLNK | |
| | MVIEKFLSDEKAHIIGIDRGERNLLYYSIIDRSGKIIDQQSLNVIDGFD | |
| | YREKLNQREIEMKDARQSWNAIGKIKDLKEGYLSKAVHEITKMAIQ | |
| | YNAIVVMEELNYGFKRGRFKVEKQIYQKFENMLIDKMNYLVFKDA | |
| | PDESPGGVLNAYQLTNPLESFAKLGKQTGILFYVPAAYTSKIDPTTG | |
| | FVNLFNTSSKTNAQERKEFLQKFESISYSAKDGGIFAFAFDYRKFGTS | |
| | KTDHKNVWTAYTNGERMRYIKEKKRNELFDPSKEIKEALTSSGIKY | |
| | DGGQNILPDILRSNNNGLIYTMYSSFIAAIQMRVYDGKEDYIISPIKN | |
| | SKGEFFRTDPKRRELPIDADANGAYNIALRGELTMRAIAEKFDPDSE | |
| | KMAKLELKHKDWFEFMQTRGD | |
| BsCpf1 | MYYQNLTKKYPVSKTIRNELIPIGKTLENIRKNNILESDVKRKQDYE | SEQ ID |
| | HVKGIMDEYHKQLINEALDNYMLPSLNQAAEIYLKKHVDVEDREE | NO. 349 |
| | FKKTQDLLRREVTGRLKEHENYTKIGKKDILDLLEKLPSISEEDYNA | |
| | LESFRNFYTYFTSYNKVRENLYSDEEKSSTVAYRLINENLPKFLDNI | |
| | KSYAFVKAAGVLADCIEEEEQDALFMVETFNMTLTQEGIDMYNYQI | |
| | GKVNSAINLYNQKNHKVEEFKKIPKMKVLYKQILSDREEVFIGEFK | |
| | DDETLLSSIGAYGNVLMTYLKSEKINIFFDALRESEGKNVYVKNDLS | |
| | KTTMSNIVFGSWSAFDELLNQEYDLANENKKKDDKYFEKRQKELK | |
| | KNKSYTLEQMSNLSKEDISPIENYIERISEDIEKICIYNGEFEKIVVNEH | |
| | DSSRKLSKNIKAVKVIKDYLDSIKELEHDIKLINGSGQELEKNLVVY | |
| | VGQEEALEQLRPVDSLYNLTRNYLTKKPFSTEKVKLNFNKSTLLNG | |
| | WDKNKETDNLGILFFKDGKYYLGIMNTTANKAFVNPPAAKTENVF | |
| | KKVDYKLLPGSNKMLPKVFFAKSNIGYYNPSTELYSNYKKGTHKK | |
| | GPSFSIDDCHNLIDFFKESIKKHEDWSKFGFEFSDTADYRDISEFYRE | |
| | VEKQGYKLTFTDIDESYINDLIEKNELYLFQIYNKDFSEYSKGKLNL | |
| | HTLYFMMLFDQRNLDNVVYKLNGEAEVFYRPASIAENELVIHKAG | |
| | EGIKNKNPNRAKVKETSTFSYDIVKDKRYSKYKFTLHIPITMNFGVD | |
| | EVRRFNDVINNALRTDDNVNVIGIDRGERNLLYVVVINSEGKILEQIS | |
| | LNSIINKEYDIETNYHALLDEREDDRNKARKDWNTIENIKELKTGYL | |
| | SQVVNVVAKLVLKYNAIICLEDLNFGFKRGRQKVEKQVYQKFEKM | |
| | LIEKLNYLVIDKSREQVSPEKMGGALNALQLTSKFKSFAELGKQSGII | |
| | YYVPAYLTSKIDPTTGFVNLFYIKYENIEKAKQFFDGFDFIRFNKKD | |
| | DMFEFSFDYKSFTQKACGIRSKWIVYTNGERIIKYPNPEKNNLFDEK | |
| | VINVTDEIKGLFKQYRIPYENGEDIKEIIISKAEADFYKRLFRLLHQTL | |
| | QMRNSTSDGTRDYIISPVKNDRGEFFCSEFSEGTMPKDADANGAYNI | |
| | ARKGLWVLEQIRQKDEGEKVNLSMTNAEWLKYAQLHLL | |
| BhCas12b | MGIHGVPAAATRSFILKIEPNEEVKKGLWKTHEVLNHGIAYYMNIL | SEQ ID |
| | KLIRQEAIYEHHEQDPKNPKKVSKAEIQAELWDFVLKMQKCNSFTH | NO. 350 |
| | EVDKDEVFNILRELYEELVPSSVEKKGEANQLSNKFLYPLVDPNSQS | |
| | GKGTASSGRKPRWYNLKIAGDPSWEEEKKKWEEDKKKDPLAKILG | |
| | KLAEYGLIPLFIPYTDSNEPIVKEIKWMEKSRNQSVRRLDKDMFIQA | |
| | LERFLSWESWNLKVKEEYEKVEKEYKTLEERIKEDIQALKALEQYE | |
| | KERQEQLLRDTLNTNEYRLSKRGLRGWREIIQKWLKMDENEPSEKY | |
| | LEVFKDYQRKHPREAGDYSVYEFLSKKENHFIWRNHPEYPYLYATF | |
| | CEIDKKKKDAKQQATFTLADPINHPLWVRFEERSGSNLNKYRILTEQ | |
| | LHTEKLKKKLTVQLDRLIYPTESGGWEEKGKVDIVLLPSRQFYNQIF | |
| | LDIEEKGKHAFTYKDESIKFPLKGTLGGARVQFDRDHLRRYPHKVE | |
| | SGNVGRIYFNMTVNIEPTESPVSKSLKIHRDDFPKVVNFKPKELTEWI | |
| | KDSKGKKLKSGIESLEIGLRVMSIDLGQRQAAAASIFEVVDQKPDIE | |
| | GKLFFPIKGTELYAVHRASFNIKLPGETLVKSREVLRKAREDNLKLM | |
| | NQKLNFLRNVLHFQQFEDITEREKRVTKWISRQENSDVPLVYQDELI | |
| | QIRELMYKPYKDWVAFLKQLHKRLEVEIGKEVKHWRKSLSDGRKG | |
| | LYGISLKNIDEIDRTRKFLLRWSLRPTEPGEVRRLEPGQRFAIDQLNH | |
| | LNALKEDRLKKMANTIIMHALGYCYDVRKKKWQAKNPACQIILFE | |
| | DLSNYNPYGERSRFENSRLMKWSRREIPRQVALQGEIYGLQVGEVG | |
| | AQFSSRFHAKTGSPGIRCRVVTKEKLQDNRFFKNLQREGRLTLDKIA | |
| | VLKEGDLYPDKGGEKFISLSKDRKCVTTHADINAAQNLQKRFWTRT | |
| | HGFYKVYCKAYQVDGQTVYIPESKDQKQKIIEEFGEGYFILKDGVY | |
| | EWVNAGKLKIKKGSSKQSSSELVDSDILKDSFDLASELKGEKLMLY | |
| | RDPSGNVFPSDKWMAAGVFFGKLERILISKLTNQYSISTIEDDSSKQS | |
| AkCas12b | MAVKSIKVKLRLSECPDILAGMWQLHRATNAGVRYYTEWVSLMR | SEQ ID |
| | QEILYSRGPDGGQQCYMTAEDCQRELLRRLRNRQLHNGRQDQPGT | NO. 351 |
| | DADLLAISRRLYEILVLQSIGKRGDAQQIASSFLSPLVDPNSKGGRGE | |
| | AKSGRKPAWQKMRDQGDPRWVAAREKYEQRKAVDPSKEILNSLD | |
| | ALGLRPLFAVFTETYRSGVDWKPLGKSQGVRTWDRDMFQQALERL | |
| | MSWESWNRRVGEEYARLFQQKMKFEQEHFAEQSHLVKLARALEA | |
| | DMRAASQGFEAKRGTAHQITRRALRGADRVFEIWKSIPEEALFSQY | |
| | DEVIRQVQAEKRRDFGSHDLFAKLAEPKYQPLWRADETFLTRYALY | |
| | NGVLRDLEKARQFATFTLPDACVNPIWTRFESSQGSNLHKYEFLFD | |
| | HLGPGRHAVRFQRLLVVESEGAKERDSVVVPVAPSGQLDKLVLREE | |
| | EKSSVALHLHDTARPDGFMAEWAGAKLQYERSTLARKARRDKQG | |
| | MRSWRRQPSMLMSAAQMLEDAKQAGDVYLNISVRVKSPSEVRGQ | |
| | RRPPYAALFRIDDKQRRVTVNYNKLSAYLEEHPDKQIPGAPGLLSGL | |
| | RVMSVDLGLRTSASISVFRVAKKEEVEALGDGRPPHYYPIHGTDDL | |
| | VAVHERSHLIQMPGETETKQLRKLREERQAVLRPLFAQLALLRLLV | |
| | RCGAADERIRTRSWQRLTKQGREFTKRLTPSWREALELELTRLEAY | |
| | CGRVPDDEWSRIVDRTVIALWRRMGKQVRDWRKQVKSGAKVKVK | |
| | GYQLDVVGGNSLAQIDYLEQQYKFLRRWSFFARASGLVVRADRES | |
| | HFAVALRQHIENAKRDRLKKLADRILMEALGYVYEASGPREGQWT | |
| | AQHPPCQLIILEELSAYRFSDDRPPSENSKLMAWGHRGILEELVNQA | |
| | QVHDVLVGTVYAAFSSRFDARTGAPGVRCRRVPARFVGATVDDSL | |
| | PLWLTEFLDKHRLDKNLLRPDDVIPTGEGEFLVSPCGEEAARVRQV | |
| | HADINAAQNLQRRLWQNFDITELRLRCDVKMGGEGTVLVPRVNNA | |
| | RAKQLFGKKVLVSQDGVTFFERSQTGGKPHSEKQTDLTDKELELIA | |
| | EADEARAKSVVLFRDPSGHIGKGHWIRQREFWSLVKQRIESHTAERI | |
| | RVRGVGSSLD | |
| BsCas12b | MAIRSIKLKLKTHTGPEAQNLRKGIWRTHRLLNEGVAYYMKMLLL | SEQ ID |
| | FRQESTGERPKEELQEELICHIREQQQRNQADKNTQALPLDKALEAL | NO. 352 |
| | RQLYELLVPSSVGQSGDAQIISRKFLSPLVDPNSEGGKGTSKAGAKP | |
| | TWQKKKEANDPTWEQDYEKWKKRREEDPTASVITTLEEYGIRPIFP | |
| | LYTNTVTDIAWLPLQSNQFVRTWDRDMLQQAIERLLSWESWNKRV | |
| | QEEYAKLKEKMAQLNEQLEGGQEWISLLEQYEENRERELRENMTA | |
| | ANDKYRITKRQMKGWNELYELWSTFPASASHEQYKEALKRVQQRL | |
| | RGRFGDAHFFQYLMEEKNRLIWKGNPQRIHYFVARNELTKRLEEAK | |
| | QSATMTLPNARKHPLWVRFDARGGNLQDYYLTAEADKPRSRRFVT | |
| | FSQLIWPSESGWMEKKDVEVELALSRQFYQQVKLLKNDKGKQKIEF | |
| | KDKGSGSTFNGHLGGAKLQLERGDLEKEEKNFEDGEIGSVYLNVVI | |
| | DFEPLQEVKNGRVQAPYGQVLQLIRRPNEFPKVTTYKSEQLVEWIK | |
| | ASPQHSAGVESLASGFRVMSIDLGLRAAAATSIFSVEESSDKNAADF | |
| | SYWIEGTPLVAVHQRSYMLRLPGEQVEKQVMEKRDERFQLHQRVK | |
| | FQIRVLAQIMRMANKQYGDRWDELDSLKQAVEQKKSPLDQTDRTF | |
| | WEGIVCDLTKVLPRNEADWEQAVVQIHRKAEEYVGKAVQAWRKR | |
| | FAADERKGIAGLSMWNIEELEGLRKLLISWSRRTRNPQEVNRFERG | |
| | HTSHQRLLTHIQNVKEDRLKQLSHAIVMTALGYVYDERKQEWCAE | |
| | YPACQVILFENLSQYRSNLDRSTKENSTLMKWAHRSIPKYVHMQAE | |
| | PYGIQIGDVRAEYSSRFYAKTGTPGIRCKKVRGQDLQGRRFENLQK | |
| | RLVNEQFLTEEQVKQLRPGDIVPDDSGELFMTLTDGSGSKEVVFLQ | |
| | ADINAAHNLQKRFWQRYNELFKVSCRVIVRDEEEYLVPKTKSVQA | |
| | KLGKGLFVKKSDTAWKDVYVWDSQAKLKGKTTFTEESESPEQLED | |
| | FQEIIEEAEEAKGTYRTLFRDPSGVFFPESVWYPQKDFWGEVKRKLY | |
| | GKLRERFLTKAR | |
| AmCas12b | MNVAVKSIKVKLMLGHLPEIREGLWHLHEAVNLGVRYYTEWLALL | SEQ ID |
| | RQGNLYRRGKDGAQECYMTAEQCRQELLVRLRDRQKRNGHTGDP | NO. 353 |
| | GTDEELLGVARRLYELLVPQSVGKKGQAQMLASGFLSPLADPKSEG | |
| | GKGTSKSGRKPAWMGMKEAGDSRWVEAKARYEANKAKDPTKQV | |
| | IASLEMYGLRPLFDVFTETYKTIRWMPLGKHQGVRAWDRDMFQQS | |
| | LERLMSWESWNERVGAEFARLVDRRDRFREKHFTGQEHLVALAQR | |
| | LEQEMKEASPGFESKSSQAHRITKRALRGADGIIDDWLKLSEGEPVD | |
| | RFDEILRKRQAQNPRRFGSHDLFLKLAEPVFQPLWREDPSFLSRWAS | |
| | YNEVLNKLEDAKQFATFTLPSPCSNPVWARFENAEGTNIFKYDFLF | |
| | DHFGKGRHGVRFQRMIVMRDGVPTEVEGIVVPIAPSRQLDALAPND | |
| | AASPIDVFVGDPAAPGAFRGQFGGAKIQYRRSALVRKGRREEKAYL | |
| | CGFRLPSQRRTGTPADDAGEVFLNLSLRVESQSEQAGRRNPPYAAV | |
| | FHISDQTRRVIVRYGEIERYLAEHPDTGIPGSRGLTSGLRVMSVDLGL | |
| | RTSAAISVFRVAHRDELTPDAHGRQPFFFPIHGMDHLVALHERSHLI | |
| | RLPGETESKKVRSIREQRLDRLNRLRSQMASLRLLVRTGVLDEQKR | |
| | DRNWERLQSSMERGGERMPSDWWDLFQAQVRYLAQHRDASGEA | |
| | WGRMVQAAVRTLWRQLAKQVRDWRKEVRRNADKVKIRGIARDV | |
| | PGGHSLAQLDYLERQYRFLRSWSAFSVQAGQVVRAERDSRFAVAL | |
| | REHIDNGKKDRLKKLADRILMEALGYVYVTDGRRAGQWQAVYPP | |
| | CQLVLLEELSEYRFSNDRPPSENSQLMVWSHRGVLEELIHQAQVHD | |
| | VLVGTIPAAFSSRFDARTGAPGIRCRRVPSIPLKDAPSIPIWLSHYLKQ | |
| | TERDAAALRPGELIPTGDGEFLVTPAGRGASGVRVVHADINAAHNL | |
| | QRRLWENFDLSDIRVRCDRREGKDGTVVLIPRLTNQRVKERYSGVI | |
| | FTSEDGVSFTVGDAKTRRRSSASQGEGDDLSDEEQELLAEADDARE | |
| | RSVVLFRDPSGFVNGGRWTAQRAFWGMVHNRIETLLAERFSVSGA | |
| | AEKVRG | |
| AaCas12b | MAVKSMKVKLRLDNMPEIRAGLWKLHTEVNAGVRYYTEWLSLLR | SEQ ID |
| | QENLYRRSPNGDGEQECYKTAEECKAELLERLRARQVENGHCGPA | NO. 354 |
| | GSDDELLQLARQLYELLVPQAIGAKGDAQQIARKFLSPLADKDAVG | |
| | GLGIAKAGNKPRWVRMREAGEPGWEEEKAKAEARKSTDRTADVL | |
| | RALADFGLKPLMRVYTDSDMSSVQWKPLRKGQAVRTWDRDMFQ | |
| | QAIERMMSWESWNQRVGEAYAKLVEQKSRFEQKNFVGQEHLVQL | |
| | VNQLQQDMKEASHGLESKEQTAHYLTGRALRGSDKVFEKWEKLD | |
| | PDAPFDLYDTEIKNVQRRNTRRFGSHDLFAKLAEPKYQALWREDAS | |
| | FLTRYAVYNSIVRKLNHAKMFATFTLPDATAHPIWTRFDKLGGNLH | |
| | QYTFLFNEFGEGRHAIRFQKLLTVEDGVAKEVDDVTVPISMSAQLD | |
| | DLLPRDPHELVALYFQDYGAEQHLAGEFGGAKIQYRRDQLNHLHA | |
| | RRGARDVYLNLSVRVQSQSEARGERRPPYAAVFRLVGDNHRAFVH | |
| | FDKLSDYLAEHPDDGKLGSEGLLSGLRVMSVDLGLRTSASISVFRV | |
| | ARKDELKPNSEGRVPFCFPIEGNENLVAVHERSQLLKLPGETESKDL | |
| | RAIREERQRTLRQLRTQLAYLRLLVRCGSEDVGRRERSWAKLIEQP | |
| | MDANQMTPDWREAFEDELQKLKSLYGICGDREWTEAVYESVRRV | |
| | WRHMGKQVRDWRKDVRSGERPKIRGYQKDVVGGNSIEQIEYLERQ | |
| | YKFLKSWSFFGKVSGQVIRAEKGSRFAITLREHIDHAKEDRLKKLAD | |
| | RIIMEALGYVYALDDERGKGKWVAKYPPCQLILLEELSEYQFNNDR | |
| | PPSENNQLMQWSHRGVFQELLNQAQVHDLLVGTMYAAFSSRFDAR | |
| | TGAPGIRCRRVPARCAREQNPEPFPWWLNKFVAEHKLDGCPLRAD | |
| | DLIPTGEGEFFVSPFSAEEGDFHQIHADLNAAQNLQRRLWSDFDISQI | |
| | RLRCDWGEVDGEPVLIPRTTGKRTADSYGNKVFYTKTGVTYYERE | |
| | RGKKRRKVFAQEELSEEEAELLVEADEAREKSVVLMRDPSGIINRG | |
| | DWTRQKEFWSMVNQRIEGYLVKQIRSRVRLQESACENTGDI | |
| RfxCas13d | MIEKKKSFAKGMGVKSTLVSGSKVYMTTFAEGSDARLEKIVEGDSI | SEQ ID |
| | RSVNEGEAFSAEMADKNAGYKIGNAKFSHPKGYAVVANNPLYTGP | NO. 355 |
| | VQQDMLGLKETLEKRYFGESADGNDNICIQVIHNILDIEKILAEYITN | |
| | AAYAVNNISGLDKDIIGFGKFSTVYTYDEFKDPEHHRAAFNNNDKLI | |
| | NAIKAQYDEFDNFLDNPRLGYFGQAFFSKEGRNYIINYGNECYDILA | |
| | LLSGLRHWVVHNNEEESRISRTWLYNLDKNLDNEYISTLNYLYDRI | |
| | TNELTNSFSKNSAANVNYIAETLGINPAEFAEQYFRFSIMKEQKNLG | |
| | FNITKLREVMLDRKDMSEIRKNHKVFDSIRTKVYTMMDFVIYRYYI | |
| | EEDAKVAAANKSLPDNEKSLSEKDIFVINLRGSFNDDQKDALYYDE | |
| | ANRIWRKLENIMHNIKEFRGNKTREYKKKDAPRLPRILPAGRDVSA | |
| | FSKLMYALTMFLDGKEINDLLTTLINKFDNIQSFLKVMPLIGVNAKF | |
| | VEEYAFFKDSAKIADELRLIKSFARMGEPIADARRAMYIDAIRILGTN | |
| | LSYDELKALADTFSLDENGNKLKKGKHGMRNFIINNVISNKRFHYLI | |
| | RYGDPAHLHEIAKNEAVVKFVLGRIADIQKKQGQNGKNQIDRYYET | |
| | CIGKDKGKSVSEKVDALTKIITGMNYDQFDKKRSVIEDTGRENAER | |
| | EKFKKIISLYLTVIYHILKNIVNINARYVIGFHCVERDAQLYKEKGYD | |
| | INLKKLEEKGFSSVTKLCAGIDETAPDKRKDVEKEMAERAKESIDSL | |
| | ESANPKLYANYIKYSDEKKAEEFTRQINREKAKTALNAYLRNTKW | |
| | NVIIREDLLRIDNKTCTLFRNKAVHLEVARYVHAYINDIAEVNSYFQ | |
| | LYHYIMQRIIMNERYEKSSGKVSEYFDAVNDEKKYNDRLLKLLCVP | |
| | FGYCIPRFKNLSIEALFDRNEAAKFDKEKKKVSGN | |
| LwCas13a | MKVTKVDGISHKKYIEEGKLVKSTSEENRTSERLSELLSIRLDIYIKN | SEQ ID |
| | PDNASEEENRIRRENLKKFFSNKVLHLKDSVLYLKNRKEKNAVQDK | NO. 356 |
| | NYSEEDISEYDLKNKNSFSVLKKILLNEDVNSEELEIFRKDVEAKLN | |
| | KINSLKYSFEENKANYQKINENNVEKVGGKSKRNIIYDYYRESAKR | |
| | NDYINNVQEAFDKLYKKEDIEKLFFLIENSKKHEKYKIREYYHKIIGR | |
| | KNDKENFAKIIYEEIQNVNNIKELIEKIPDMSELKKSQVFYKYYLDKE | |
| | ELNDKNIKYAFCHFVEIEMSQLLKNYVYKRLSNISNDKIKRIFEYQN | |
| | LKKLIENKLLNKLDTYVRNCGKYNYYLQVGEIATSDFIARNRQNEA | |
| | FLRNIIGVSSVAYFSLRNILETENENGITGRMRGKTVKNNKGEEKYV | |
| | SGEVDKIYNENKQNEVKENLKMFYSYDFNMDNKNEIEDFFANIDEA | |
| | ISSIRHGIVHFNLELEGKDIFAFKNIAPSEISKKMFQNEINEKKLKLKIF | |
| | KQLNSANVFNYYEKDVIIKYLKNTKFNFVNKNIPFVPSFTKLYNKIE | |
| | DLRNTLKFFWSVPKDKEEKDAQIYLLKNIYYGEFLNKFVKNSKVFF | |
| | KITNEVIKINKQRNQKTGHYKYQKFENIEKTVPVEYLAIIQSREMINN | |
| | QDKEEKNTYIDFIQQIFLKGFIDYLNKNNLKYIESNNNNDNNDIFSKI | |
| | KIKKDNKEKYDKILKNYEKHNRNKEIPHEINEFVREIKLGKILKYTE | |
| | NLNMFYLILKLLNHKELTNLKGSLEKYQSANKEETFSDELELINLLN | |
| | LDNNRVTEDFELEANEIGKFLDFNENKIKDRKELKKFDTNKIYFDGE | |
| | NIIKHRAFYNIKKYGMLNLLEKIADKAKYKISLKELKEYSNKKNEIE | |
| | KNYTMQQNLHRKYARPKKDEKFNDEDYKEYEKAIGNIQKYTHLKN | |
| | KVEFNELNLLQGLLLKILHRLVGYTSIWERDLRFRLKGEFPENHYIE | |
| | EIFNFDNSKNVKYKSGQIVEKYINFYKELYKDNVEKRSIYSDKKVK | |
| | KLKQEKKDLYIRNYIAHFNYIPHAEISLLEVLENLRKLLSYDRKLKN | |
| | AIMKSIVDILKEYGFVATFKIGADKKIEIQTLESEKIVHLKNLKKKKL | |
| | MTDRNSEELCELVKVMFEYKALE | |
| PspCas13b | MNIPALVENQKKYFGTYSVMAMLNAQTVLDHIQKVADIEGEQNEN | SEQ ID |
| | NENLWFHPVMSHLYNAKNGYDKQPEKTMFIIERLQSYFPFLKIMAE | NO. 357 |
| | NQREYSNGKYKQNRVEVNSNDIFEVLKRAFGVLKMYRDLTNHYKT | |
| | YEEKLNDGCEFLTSTEQPLSGMINNYYTVALRNMNERYGYKTEDL | |
| | AFIQDKRFKFVKDAYGKKKSQVNTGFFLSLQDYNGDTQKKLHLSG | |
| | VGIALLICLFLDKQYINIFLSRLPIFSSYNAQSEERRIIIRSFGINSIKLPK | |
| | DRIHSEKSNKSVAMDMLNEVKRCPDELFTTLSAEKQSRFRIISDDHN | |
| | EVLMKRSSDRFVPLLLQYIDYGKLFDHIRFHVNMGKLRYLLKADKT | |
| | CIDGQTRVRVIEQPLNGFGRLEEAETMRKQENGTFGNSGIRIRDFEN | |
| | MKRDDANPANYPYIVDTYTHYILENNKVEMFINDKEDSAPLLPVIE | |
| | DDRYVVKTIPSCRMSTLEIPAMAFHMFLFGSKKTEKLIVDVHNRYK | |
| | RLFQAMQKEEVTAENIASFGIAESDLPQKILDLISGNAHGKDVDAFI | |
| | RLTVDDMLTDTERRIKRFKDDRKSIRSADNKMGKRGFKQISTGKLA | |
| | DFLAKDIVLFQPSVNDGENKITGLNYRIMQSAIAVYDSGDDYEAKQ | |
| | QFKLMFEKARLIGKGTTEPHPFLYKVFARSIPANAVEFYERYLIERKF | |
| | YLTGLSNEIKKGNRVDVPFIRRDQNKWKTPAMKTLGRIYSEDLPVE | |
| | LPRQMFDNEIKSHLKSLPQMEGIDFNNANVTYLIAEYMKRVLDDDF | |
| | QTFYQWNRNYRYMDMLKGEYDRKGSLQHCFTSVEEREGLWKERA | |
| | SRTERYRKQASNKIRSNRQMRNASSEEIETILDKRLSNSRNEYQKSE | |
| | KVIRRYRVQDALLFLLAKKTLTELADFDGERFKLKEIMPDAEKGILS | |
| | EIMPMSFTFEKGGKKYTITSEGMKLKNYGDFFVLASDKRIGNLLELV | |
| | GSDIVSKEDIMEEFNKYDQCRPEISSIVFNLEKWAFDTYPELSARVD | |
| | REEKVDFKSILKILLNNKNINKEQSDILRKIRNAFDHNNYPDKGVVEI | |
| | KALPEIAMSIKKAFGEYAIMKGSLQ | |
| PguCas13b | MTEQSERPYNGTYYTLEDKHFWAAFLNLARHNAYITLTHIDRQLAY | SEQ ID |
| | SKADITNDQDVLSFKALWKNFDNDLERKSRLRSLILKHFSFLEGAA | NO. 358 |
| | YGKKLFESKSSGNKSSKNKELTKKEKEELQANALSLDNLKSILFDFL | |
| | QKLKDFRNYYSHYRHSGSSELPLFDGNMLQRLYNVFDVSVQRVKI | |
| | DHEHNDEVDPHYHFNHLVRKGKKDRYGHNDNPSFKHHFVDGEGM | |
| | VTEAGLLFFVSLFLEKRDAIWMQKKIRGFKGGTETYQQMTNEVFCR | |
| | SRISLPKLKLESLRMDDWMLLDMLNELVRCPKPLYDRLREDDRACF | |
| | RVPVDILPDEDDTDGGGEDPFKNTLVRHQDRFPYFALRYFDLKKVF | |
| | TSLRFHIDLGTYHFAIYKKMIGEQPEDRHLTRNLYGFGRIQDFAEEH | |
| | RPEEWKRLVRDLDYFETGDKPYISQTSPHYHIEKGKIGLRFMPEGQH | |
| | LWPSPEVGTTRTGRSKYAQDKRLTAEAFLSVHELMPMMFYYFLLR | |
| | EKYSEEVSAERVQGRIKRVIEDVYAVYDAFARDEINTRDELDACLA | |
| | DKGIRRGHLPRQMIAILSQEHKDMEEKIRKKLQEMMADTDHRLDM | |
| | LDRQTDRKIRIGRKNAGLPKSGVIADWLVRDMMRFQPVAKDASGK | |
| | PLNNSKANSTEYRMLQRALALFGGEKERLTPYFRQMNLTGGNNPH | |
| | PFLHETRWESHTNILSFYRSYLRARKAFLERIGRSDRVENRPFLLLKE | |
| | PKTDRQTLVAGWKGEFHLPRGIFTEAVRDCLIEMGHDEVASYKEVG | |
| | FMAKAVPLYFERACEDRVQPFYDSPFNVGNSLKPKKGRFLSKEERA | |
| | EEWERGKERFRDLEAWSYSAARRIEDAFAGIEYASPGNKKKIEQLL | |
| | RDLSLWEAFESKLKVRADRINLAKLKKEILEAQEHPYHDFKSWQKF | |
| | ERELRLVKNQDIITWMMCRDLMEENKVEGLDTGTLYLKDIRPNVQ | |
| | EQGSLNVLNRVKPMRLPVVVYRADSRGHVHKEEAPLATVYIEERD | |
| | TKLLKQGNFKSFVKDRRLNGLFSFVDTGGLAMEQYPISKLRVEYEL | |
| | AKYQTARVCVFELTLRLEESLLTRYPHLPDESFREMLESWSDPLLAK | |
| | WPELHGKVRLLIAVRNAFSHNQYPMYDEAVFSSIRKYDPSSPDAIEE | |
| | RMGLNIAHRLSEEVKQAKETVERIIQAGSLQ | |
| RanCas13b | MEKPLLPNVYTLKHKFFWGAFLNIARHNAFITICHINEQLGLKTPSN | SEQ ID |
| | DDKIVDVVCETWNNILNNDHDLLKKSQLTELILKHFPFLTAMCYHP | NO. 359 |
| | PKKEGKKKGHQKEQQKEKESEAQSQAEALNPSKLIEALEILVNQLH | |
| | SLRNYYSHYKHKKPDAEKDIFKHLYKAFDASLRMVKEDYKAHFTV | |
| | NLTRDFAHLNRKGKNKQDNPDFNRYRFEKDGFFTESGLLFFTNLFL | |
| | DKRDAYWMLKKVSGFKASHKQREKMTTEVFCRSRILLPKLRLESR | |
| | YDHNQMLLDMLSELSRCPKLLYEKLSEENKKHFQVEADGFLDEIEE | |
| | EQNPFKDTLIRHQDRFPYFALRYLDLNESFKSIRFQVDLGTYHYCIY | |
| | DKKIGDEQEKRHLTRTLLSFGRLQDFTEINRPQEWKALTKDLDYKE | |
| | TSNQPFISKTTPHYHITDNKIGFRLGTSKELYPSLEIKDGANRIAKYPY | |
| | NSGFVAHAFISVHELLPLMFYQHLTGKSEDLLKETVRHIQRIYKDFE | |
| | EERINTIEDLEKANQGRLPLGAFPKQMLGLLQNKQPDLSEKAKIKIE | |
| | KLIAETKLLSHRLNTKLKSSPKLGKRREKLIKTGVLADWLVKDFMR | |
| | FQPVAYDAQNQPIKSSKANSTEFWFIRRALALYGGEKNRLEGYFKQ | |
| | TNLIGNTNPHPFLNKFNWKACRNLVDFYQQYLEQREKFLEAIKNQP | |
| | WEPYQYCLLLKIPKENRKNLVKGWEQGGISLPRGLFTEAIRETLSED | |
| | LMLSKPIRKEIKKHGRVGFISRAITLYFKEKYQDKHQSFYNLSYKLE | |
| | AKAPLLKREEHYEYWQQNKPQSPTESQRLELHTSDRWKDYLLYKR | |
| | WQHLEKKLRLYRNQDVMLWLMTLELTKNHFKELNLNYHQLKLEN | |
| | LAVNVQEADAKLNPLNQTLPMVLPVKVYPATAFGEVQYHKTPIRT | |
| | VYIREEHTKALKMGNFKALVKDRRLNGLFSFIKEENDTQKHPISQLR | |
| | LRRELEIYQSLRVDAFKETLSLEEKLLNKHTSLSSLENEFRALLEEW | |
| | KKEYAASSMVTDEHIAFIASVRNAFCHNQYPFYKEALHAPIPLFTVA | |
| | QPTTEEKDGLGIAEALLKVLREYCEIVKSQIGSSLQKKLEELELGSS | |

In some embodiments of the gene editing system described herein, at least one of the tracrRNA is any one of SEQ ID NO: 10-12. In some embodiments, the tracrRNA of is any one of SEQ ID NOs: 22-24.

In some embodiments of the gene editing system described herein, the first protein-binding RNA motif and the first RNA binding domain, the second protein-binding RNA motif and the second RNA binding domain, and the third protein-binding RNA motif and the third RNA binding domain, are each independently selected from the group consisting of a MS2 phage operator stem-loop and MS2 coat protein (MCP) or an RNA-binding section thereof, a boxB and N22p or an RNA-binding section thereof, a telomerase Ku binding motif and Ku protein or an RNA-binding section thereof, a telomerase Sm7 binding motif and Sm7 protein or an RNA-binding section thereof, a PP7 phage operator stem-loop and PP7 coat protein (PCP) or an RNA-binding section thereof, a SfMu phage Coin stem-loop and Coin RNA binding protein or an RNA-binding section thereof, and an RNA aptamer and corresponding aptamer ligand or an RNA-binding section thereof. In some embodiments, the protein-binding RNA motif and the RNA binding domain are the variants of those disclosed above.

TABLE 7

| Name | Sequence | SEQ ID NO |
|---|---|---|
| MS2 | ACAUGAGGAUCACCCAUGU | SEQ ID |
| | | NO: 139 |
| PP7 | GGAGCAGACGAUAUGGCGUCGCUCC | SEQ ID |
| | | NO: 140 |
| boxB | GCCCUGAAGAAGGGC | SEQ ID |
| | | NO: 141 |
| MS2 coat | MASNFTQFVLVDNGGTGDVTVAPSNFANGIAEWISSN | SEQ ID |
| protein | SRSQAYKVTCSVRQSSAQNRKYTIKVEVPKGAWRSY | NO: 48 |
| (MCP) | LNMELTIPIFATNSDCELIVKAMQGLLKDGNPIPSAIAA | |
| | NSGIY | |
| PP7 coat | MGSKTIVLSVGEATRTLTEIQSTADRQIFEEKVGPLVG | SEQ ID |
| protein | RLRLTASLRQNGAKTAYRVNLKLDQADVVDSGLPKV | NO: 49 |
| (PCP) | RYTQVWSHDVTIVANSTEASRKSLYDLTKSLVATSQV | |
| | EDLVVNLVPLGR | |
| boxB coat | MGNARTRRRERRAEKQAQWKAAN | SEQ ID |
| protein | | NO: 50 |
| (N22p) | | |
| Telomerase | UUGUGUUUCUACUUAUAGAUGGCUAAAAUCUGAG | SEQ ID |
| Ku binding | UUUAGAAAAUGCAA | NO: 142 |
| motif | | |
| Telomerase | AAUUUUUGGA | SEQ ID |
| Sm7 binding | | NO: 143 |
| motif | | |
| SfMu | CUGAAUGCCUGCGAGCAUC | SEQ ID |
| | | NO: 144 |
| KU70 | MELDPDDVFRDEDEDPENDFFQEKEASKEFVVYLIDA | SEQ ID |
| | SPKMFCSTCPSEEEDKQESHFHIAVSCIAQSLKAHIINR | NO: 145 |
| | SNDEIAICFFNTREKKNLQDLNGVYVFNVPERDSIDRP | |
| | TARLIKEFDLIEESFDKEIGSQTGIVSDSRENSLYSALW | |
| | VAQALLRKGSLKTADKRMFLFTNEDDPFGSMRISVKE | |
| | DMTRTTLQRAKDAQDLGISIELLPLSQPDKQFNITLFY | |
| | KDLIGLNSDELTEFMPSVGQKLEDMKDQLKKRVLAK | |
| | RIAKRITFVICDGLSIELNGYALLRPAIPGSITWLDSTTN | |
| | LPVKVERSYICTDTGAIMQDPIQRIQPYKNQNIMFTVE | |
| | ELSQVKRISTGHLRLLGFKPLSCLKDYHNLKPSTFLYP | |
| | SDKEVIGSTRAFIALHRSMIQLERFAVAFYGGTTPPRL | |
| | VALVAQDEIESDGGQVEPPGINMIYLPYANDIRDIDEL | |
| | HSKPGVAAPRASDDQLKKASALMRRLELKDFSVCQF | |
| | ANPALQRHYAILQAIALDENELRETRDETLPDEEGMN | |
| | RPAVVKAIEQFKQSIYGDDPDEESDSGAKEKSKKRKA | |
| | GDADDGKYDYIELAKTGKLKDLTVVELKTYLTANNL | |
| | LVSGKKEVLINRILTHIGK | |
| KU80 | MSSESTTFIVDVSPSMMKNNNVSKSMAYLEYTLLNKS | SEQ ID |
| | KKSRKTDWISCYLANCPVSENSQEIPNVFQIQSFLAPV | NO: 146 |
| | TTTATIGFIKRLKQYCDQHSHDSSNEGLQSMIQCLLVV | |
| | SLDIKQQFQARKILKQIVVFTDNLDDLDITDEEIDLLTE | |
| | ELSTRIILIDCGKDTQEERKKSNWLKLVEAIPNSRIYN | |
| | MNELLVEITSPATSVVKPVRVFSGELRLGADILSTQTS | |
| | NPSGSMQDENCLCIKVEAFPATKAVSGLNRKTAVEVE | |
| | DSQKKERYVGVKSIIEYEIHNEGNKKNVSEDDQSGSS | |
| | YIPVTISKDSVTKAYRYGADYVVLPSVLVDQTVYESF | |
| | PGLDLRGFLNREALPRYFLTSESSFITADTRLGCQSDL | |
| | MAFSALVDVMLENRKIAVARYVSKKDSEVNMCALCP | |
| | VLIEHSNINSEKKFVKSLTLCRLPFAEDERVTDFPKLLD | |
| | RTTTSGVPLKKETDGHQIDELMEQFVDSMDTDELPEIP | |
| | LGNYYQPIGEVTTDTTLPLPSLNKDQEENKKDPLRIPT | |
| | VFVYRQQQVLLEWIHQLMINDSREFEIPELPDSLKNKI | |
| | SPYTHKKFDSTKLVEVLGIKKVDKLKLDSELKTELER | |
| | EKIPDLETLLKRGEQHSRGSPNNSNN | |
| Sm7-like | GSVIDVSSQRVNVQRPLDALGNSLNSPVIIKLKGDREF | SEQ ID |
| protein | RGVLKSFDLHMNLVLNDAEELEDGEVTRRLGT | NO: 147 |
| SfMu Com | MKSIRCKNCNKLLFKADSFDHIEIRCPRCKRHIIMLNA | SEQ ID |
| binding | CEHPTEKHCGKREKITHSDETVRY | NO: 148 |
| protein | | |

For any protein of the present disclosure, biological equivalents thereof are also provided. In some embodiments, the biological equivalents have at least about 70%, 75%, 80%, 85%, 90%, 95%, 98%, or 99% sequence identity with the reference protein. Preferably, the biological equivalents retain the desired activity of the reference protein. In some embodiments, the biological equivalents are derived by including one, two, three, four, five, or more amino acid additions, deletions, substitutions, or the combinations thereof. In some embodiments, the substitution is a conservative amino acid substitution.

### Polynucleotides

In an aspect, the present disclosure provides a polynucleotide comprising a sequence encoding the engineered crRNA described herein. In another aspect, the present disclosure provides a polynucleotide comprising a sequence encoding the engineered tracrRNA described herein.

In an aspect, the present disclosure provides a polynucleotide comprising a sequence encoding all components except the first and second Cas proteins in the gene editing system described herein.

In an aspect, the present disclosure provides a kit comprising the polynucleotide which comprises a sequence encoding all components except the first and second Cas proteins in the gene editing system described herein, and a polynucleotide encoding the first and/or the second Cas protein in any one of the gene editing systems described herein.

The polynucleotides disclosed herein can be obtained by methods known in the art. For example, the polynucleotide can be obtained from cloned DNA (e.g., from a DNA library), by chemical synthesis, by cDNA cloning, or by the cloning of genomic DNA or fragments thereof, purified from the desired cell. When the polynucleotides are produced by recombinant means, any method known to those skilled in the art for identification of nucleic acids that encode desired genes can be used. Any method available in the art can be used to obtain a full length (i.e., encompassing the entire coding region) cDNA or genomic DNA encoding a desired protein, such as from a cell or tissue source. Modified or variant polynucleotides can be engineered from a wildtype polynucleotide using standard recombinant DNA methods. Polynucleotides can be cloned or isolated using any available methods known in the art for cloning and isolating nucleic acid molecules. Such methods include PCR amplification of nucleic acids and screening of libraries, including nucleic acid hybridization screening, antibody-based screening, and activity-based screening.

Methods for amplification of polynucleotides can be used to isolate polynucleotides encoding a desired protein, including for example, polymerase chain reaction (PCR) methods. PCR can be carried out using any known methods or procedures in the art. Exemplary methods include use of a Perkin-Elmer Cetus thermal cycler and Taq polymerase (Gene Amp). A nucleic acid containing gene of interest can be used as a source material from which a desired polypeptide-encoding nucleic acid molecule can be amplified. For example, DNA and mRNA preparations, cell extracts, tissue extracts from an appropriate source (e.g., testis, prostate, breast), fluid samples (e.g., blood, serum, saliva), samples from healthy and/or diseased subjects can be used in amplification methods. The source can be from any eukaryotic species including, but not limited to, vertebrate, mammalian, human, porcine, bovine, feline, avian, equine, canine, and other primate sources. Nucleic acid libraries also can be used as a source material. Primers can be designed to amplify a desired polynucleotide. For example, primers can be designed based on expressed sequences from which a desired polynucleotide is generated. Primers can be designed based on back-translation of a polypeptide amino acid sequence. If desired, degenerate primers can be used for amplification. Oligonucleotide primers that hybridize to sequences at the 3′ and 5′ termini of the desired sequence can be uses as primers to amplify by PCR from a nucleic acid sample. Primers can be used to amplify the entire full-length polynucleotide, or a truncated sequence thereof. Nucleic acid molecules generated by amplification can be sequenced and confirmed to encode a desired polypeptide.

### Vectors

In an aspect, the present disclosure provides a vector comprising the polynucleotide described herein.

In an aspect, the present disclosure provides a vector comprising the polynucleotide described herein.

In some embodiments of the vector described herein, the vector is a plasmid or a viral vector.

In some embodiments of the vector described herein, the vector is a polycistronic vector.

In an aspect, the present disclosure provides a kit comprising the vector described herein, and a vector comprising the polynucleotide encoding the first and/or second Cas protein in any one of the gene editing systems described herein.

Any methods known in the art for the insertion of DNA fragments into a vector can be used to construct expression vectors comprising a polynucleotide disclosed herein. These methods can include in vitro recombinant DNA and synthetic techniques and in vivo (genetic) recombination. The polynucleotide disclosed herein can be operably linked to control sequences in the expression vector(s) to ensure protein expression. Such control sequences may include, but are not limited to, leader or signal sequences, promoters (e.g., naturally associated or heterologous promoters), ribosomal binding sites, enhancer or activator elements, translational start and termination sequences, and transcription start and termination sequences, and are chosen to be compatible with the host cell chosen to express the proteins. Constitutive or inducible promoters as known in the art are also contemplated. The promoters may be either naturally occurring promoters, hybrid promoters that combine elements of more than one promoter, or synthetic promoters. An expression construct may be present in a cell on an episome, such as a plasmid, or the expression construct may be inserted in a chromosome such as in a gene locus. In some embodiment, the expression vector includes a selectable marker gene to allow the selection of transformed host cells. In some embodiments, the vector is an expression vector comprising a nucleotide sequence encoding a variant polypeptide operably linked to at least one regulatory control sequence. Regulatory control sequences for use herein include promoters, enhancers, and other expression control elements. In some embodiments, the expression vector is designed for the choice of the host cell to be transformed, the particular variant polypeptide desired to be expressed, the vector's copy number, the ability to control that copy number, and/or the expression of any other protein encoded by the vector, such as antibiotic markers.

The vector can include, but is not limited to, viral vectors and plasmid DNA. Viral vectors can include, but are not limited to, adenoviral vectors, lentiviral vectors, retroviral vectors, and adeno-associated viral vectors. Commonly, expression vectors contain selection markers such as ampicillin-resistance, hygromycin-resistance, tetracycline resistance, kanamycin resistance, or neomycin resistance to permit detection of those cells transformed with the desired DNA sequences. Suitable vectors, promoter, and enhancer elements are known in the art; many are commercially available for generating subject recombinant constructs. In some embodiments, the vector is a polycistronic vector. In some embodiments, the vector is a bicistronic vector or a tricistronic vector. Bicistronic or polycistronic expression vectors may include (1) multiple promoters fused to each of the open reading frames; (2) insertion of splicing signals between genes; (3) fusion of genes whose expressions are driven by a single promoter; and (4) insertion of proteolytic cleavage sites between genes (self-cleavage peptide) or insertion of internal ribosomal entry sites (IRESs) between genes.

A polycistronic vector is used to co-express multiple genes in the same cell. Two strategies are most commonly used to construct a multicistronic vector. First, an Internal Ribosome Entry Site (IRES) element is typically used for bi-cistronic vectors. The IRES element, acting as another ribosome recruitment site, allows initiation of translation from an internal region of the mRNA. Thus, two proteins are translated from one mRNA. IRES elements are quite large (usually 500-600 bp) (Pelletier et al., 1988; Jang et al., 1988). The engineered CD47 proteins disclosed herein have a smaller size compared to the wild-type full-length human CD47, and thus could be used with IRES element in a multicistronic vectors having limited packaging capacity.

### Cells

In an aspect, the present disclosure provides a cell comprising the engineered crRNA described herein.

In an aspect, the present disclosure provides a cell comprising the gene editing system described herein.

In an aspect, the present disclosure provides a cell comprising the polynucleotide described herein.

In some embodiments of the cell in claim 61, further comprising a polynucleotide encoding the first and/or the second Cas protein described herein.

In an aspect, the present disclosure provides a cell comprising the vector described herein.

In some embodiments of the cell described herein, the cell comprises a vector described herein, and a vector comprising a polynucleotide encoding the first and/or the second Cas protein in the gene editing system described herein.

In some embodiments of the cell described herein, wherein the cell is a stem cell, a somatic cell, a blood cell, or an immune cell.

In some embodiments of the cell described herein, wherein the cell is a primary cell or a differentiated cell.

In some embodiments of the cell described herein, wherein the cell is a human cell.

In some embodiments, the cell is selected from, but not limited to, stem cells, pluripotent cells, somatic cells, cardiac cells, cardiac progenitor cells, neural cells, glial progenitor cells, endothelial cells, T cells, B cells, pancreatic islet cells, retinal pigmented epithelium cells, hepatocytes, thyroid cells, skin cells, blood cells, plasma cells, platelets, renal cells, epithelial cells, CAR-T cells, NK cells, and CAR-NK cells. In some embodiments, the cell is from a mammal. In some embodiments, the cell is human cell.

In some embodiments, the cell is a primary cell. Primary cells are isolated directly from human or animal tissue using enzymatic or mechanical methods. Once isolated, they are placed in an artificial environment in plastic or glass containers supported with specialized medium containing essential nutrients and growth factors to support proliferation. Primary cells could be of two types: adherent or suspension. Adherent cells require attachment for growth and are said to be anchorage-dependent cells. Adherent cells are usually derived from tissues of organs. Suspension cells do not require attachment for growth and are said to be anchorage-independent cells. Most suspension cells are isolated from the blood system, but some tissue-derived cells can also be used in suspension, such as hepatocytes or intestinal cells. Although primary cells usually have a limited lifespan, they offer a number of advantages compared to cell lines. Primary cell culture enables researchers to study donors and not just cells. Several factors such as age, medical history, race, and sex can be considered when building an experimental model. With a growing trend towards personalized medicine, such donor variability and tissue complexity can be achieved with use of primary cells, but are difficult to replicate with cell lines that are more systematic and uniform in nature and do not capture the true diversity of a living tissue.

In some embodiments, the cell is a differentiated cell. Differentiated cells are cells that have undergone differentiation. They are mature cells that perform a specialized function. Some examples of differentiated cells are epithelial cells, skin fibroblasts, endothelial cells lining the blood vessels, smooth muscle cells, liver cells, nerve cells, human cardiac muscle cells, etc. Generally, these cells have a unique morphology, metabolic activity, membrane potential, and responsiveness to signals facilitating their function in a body tissue or organ.

### Compositions

In another aspect, the present disclosure provides a composition comprising the gene editing system disclosed herein.

In another aspect, the present disclosure provides a composition comprising the cell disclosed herein.

As used herein, the term “composition” includes, but is not limited to, a pharmaceutical composition. A “pharmaceutical composition” refers to an active pharmaceutical agent formulated in pharmaceutically acceptable or physiologically acceptable solutions for administration to a cell or an animal, either alone, or in combination with one or more other modalities of therapy. It will also be understood that, if desired, the compositions of the disclosure may be administered in combination with other agents, such as, e.g., cytokines, growth factors, hormones, small molecules, chemotherapeutics, pro-drugs, drugs, antibodies, or other various pharmaceutically active agents. There is virtually no limit to other components that may also be included in the compositions, provided that the additional agents do not adversely affect the ability of the composition to deliver the intended therapy. The phrase “pharmaceutically acceptable” is used herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The compositions may also comprise a pharmaceutically acceptable carrier, diluent, or excipient. As used herein “pharmaceutically acceptable carrier, diluent, or excipient” includes, without limitation, any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, or emulsifier which has been approved by the United States Food and Drug Administration as being acceptable for use in humans or domestic animals. Exemplary pharmaceutically acceptable carriers include, but are not limited to, to sugars, such as lactose, glucose, and sucrose; starches, such as corn starch and potato starch; cellulose, and its derivatives, such as sodium carboxymethyl cellulose, ethyl cellulose, and cellulose acetate; tragacanth; malt; gelatin; talc; cocoa butter; waxes; animal and vegetable fats; paraffins; silicones; bentonites; silicic acid; zinc oxide; oils, such as peanut oil, cottonseed oil, safflower oil, sesame oil, olive oil, corn oil, and soybean oil; glycols, such as propylene glycol; polyols, such as glycerin, sorbitol, mannitol, and polyethylene glycol; esters, such as ethyl oleate, and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical formulations.

The liquid pharmaceutical compositions, whether they be solutions, suspensions or other like form, may include one or more of the following: sterile diluents such as water for injection, saline solution, preferably physiological saline; Ringers solution; isotonic sodium chloride; fixed oils such as synthetic mono or diglycerides which may serve as the solvent or suspending medium; polyethylene glycols; glycerin; propylene glycol or other solvents; antibacterial agents, such as benzyl alcohol or methyl paraben; antioxidants such as ascorbic acid or sodium bisulfite; chelating agents, such as ethylenediaminetetraacetic acid; buffers such as acetates, citrates, or phosphates; and agents for the adjustment of tonicity, such as sodium chloride or dextrose. The parenteral preparation can be enclosed in ampoules, disposable syringes, or multiple dose vials made of glass or plastic. An injectable pharmaceutical composition is preferably sterile.

The composition may be suitably developed for intravenous, intratumoral, oral, rectal, vaginal, parenteral, topical, pulmonary, intranasal, buccal, ophthalmic, or another route of administration.

### Methods

In another aspect, the present disclosure provides a method for editing a target gene in a cell, comprising administering the gene editing system disclosed herein into the cell.

In another aspect, the present disclosure provides a method for editing a target gene in a cell, comprising administering the polynucleotides disclosed herein into the cell.

In another aspect, the present disclosure provides a method for editing a target gene in a cell, comprising administering the vectors disclosed herein into the cell.

In another aspect, the present disclosure provides a method for reducing low-density lipoprotein cholesterol (LDL-C) in a subject by editing the PCSK9 gene in the subject, comprising administering to the subject the gene editing system disclosed herein, wherein the hcrRNA and the mcrRNA are SEQ ID NO: 302 and SEQ ID NO: 303, respectively; or SEQ ID NO: 304 and SEQ ID NO: 305, respectively; or SEQ ID NO: 306 and SEQ ID NO: 307, respectively; or SEQ ID NO: 370 and SEQ ID NO: 371, respectively; or SEQ ID NO: 372 and SEQ ID NO: 373, respectively; or SEQ ID NO: 374 and SEQ ID NO: 375, respectively. In some embodiments, the gene editing system is delivered into the subject by lipid nanoparticles (LNP).

In another aspect, the present disclosure provides a method for reducing low-density lipoprotein cholesterol (LDL-C) and triglyceride in a subject by editing the ANGPTL3 gene in the subject, comprising administering to the subject the gene editing system disclosed herein, wherein the hcrRNA and the mcrRNA are SEQ ID NO: 364 and SEQ ID NO: 365, respectively; or SEQ ID NO: 366 and SEQ ID NO: 367, respectively; or SEQ ID NO: 368 and SEQ ID NO: 369, respectively; or SEQ ID NO: 376 and SEQ ID NO: 377, respectively; or SEQ ID NO: 378 and SEQ ID NO: 379, respectively; or SEQ ID NO: 380 and SEQ ID NO: 381, respectively. In some embodiments, the gene editing system is delivered into the subject by lipid nanoparticles (LNP).

Proprotein convertase subtilisin/kexin type 9 (PCSK9) is a protein that plays a significant role in cholesterol regulation, particularly in the metabolism of low-density lipoprotein cholesterol (LDL-C). PCSK9 is produced primarily in the liver and binds to LDL receptors (LDLR) on the surface of liver cells. LDLR is responsible for removing LDL-C from the bloodstream by internalizing it into liver cells, where it's broken down and cleared. When PCSK9 binds to LDLR, it promotes the degradation of the receptor. Fewer receptors mean less LDL-C is removed from the bloodstream, leading to higher levels of LDL-C in the blood. Inhibiting or reducing the expression of PCSK9 reduces the degradation of LDLR With more LDL receptors available, the liver cells can efficiently remove more LDL-C from the blood.

Angiopoietin-like 3 (ANGPTL3) is another attractive target for lipid lowering. By mainly affecting triglyceride-rich lipoproteins, ANGPTL3 reduction may prove complementary to LDL-C lowering with PCSK9 blockade. A therapy targeting ANGPTL3 protein is already approved for the treatment of homozygous familial hypercholesterolemia, which reduces LDL-C in these patients in an LDLR-independent mechanism.

TABLE 8

| Name | Sequence | SEQ ID NO. |
|---|---|---|
| hcrRNA-1 | GUUUGAGAGCUAGGCCAACAUGAGGAUCACCCAUG | SEQ ID NO: 1 |
| | UCUGCAGGGCCUGCUGUUUUG | |
| hcrRNA-2 | GUUUGAGAGCUAUGCUGGGCCAACAUGAGGAUCAC | SEQ ID NO: 2 |
| | CCAUGUCUGCAGGGCCUUUUG | |
| hcrRNA-3 | GUUUGAGAGCUAUGCUGUUUUGGGCCAACAUGAGG | SEQ ID NO: 3 |
| | AUCACCCAUGUCUGCAGGGCC | |
| mcrRNA-1 | GUUUGAGAGCUAGGGCCCUGAAGAAGGGCCCUGCU | SEQ ID NO: 4 |
| | GUUUUG | |
| mcrRNA-2 | GUUUGAGAGCUAUGCUGGGGCCCUGAAGAAGGGCC | SEQ ID NO: 5 |
| | CUUUUG | |
| mcrRNA-3 | GUUUGAGAGCUAUGCUGUUUUGGGGCCCUGAAGAA | SEQ ID NO: 6 |
| | GGGCCC | |
| mcrRNA-1- | GUUUGAGAGCUAGGGCCCUGAAGAAGGGCGGGCCC | SEQ ID NO: 7 |
| 2xboxB | UGAAGAAGGGCCCAACCUGCUGUUUUG | |
| mcrRNA-O | GUUUGAGAGCUAUGCUGUUUUG | SEQ ID NO: 8 |
| hcrRNA-O | GUUUGAGAGCUAUGCUGUUUUG | SEQ ID NO: 9 |
| tracrRNA-O, 3 | GGAACCAUUCAAAACAGCAUAGCAAGUUCAAAUAA | SEQ ID NO: |
| | GGCUAGUCCGUUAUCAACUUGAAAAAGUGGCACCG | 10 |
| | AGUCGGUGCUUUUUUU | |
| tracrRNA-1 | GGAACCAUUCAAAACAGCAAAAUAGCAAGUUCAAA | SEQ ID NO: |
| | UAAGGCUAGUCCGUUAUCAACUUGAAAAAGUGGCA | 11 |
| | CCGAGUCGGUGCUUUUUUU | |
| tracrRNA-2 | GGAACCAUUCAAAAAAACAGCAUAGCAAGUUCAAA | SEQ ID NO: |
| | UAAGGCUAGUCCGUUAUCAACUUGAAAAAGUGGCA | 12 |
| | CCGAGUCGGUGCUUUUUUU | |
| HBG-hcrRNA-1 | mA*mC*mU*CCACCCAGUUUGAGAGCUAGGCCAACA | SEQ ID NO: |
| | UGAGGAUCACCCAUGUCUGCAGGGCCUGCUGUU*m | 13 |
| | U*mU*mG | |
| HBG-hcrRNA-2 | mA*mC*mU*CCACCCAGUUUGAGAGCUAUGCUGGGC | SEQ ID NO: |
| | CAACAUGAGGAUCACCCAUGUCUGCAGGGCCUU*m | 14 |
| | U*mU*mG | |
| HBG-hcrRNA-3 | mA*mC*mU*CCACCCAGUUUGAGAGCUAUGCUGUUU | SEQ ID NO: |
| | UGGGCCAACAUGAGGAUCACCCAUGUCUGCAGG*m | 15 |
| | G*mC*mC | |
| HBG-mcrRNA-1 | mC*mU*mU*GACCAAUAGCCUUGACAGUUUGAGAGC | SEQ ID NO: |
| | UAGGGCCCUGAAGAAGGGCCCUGCUGUU*mU*mU*m | 16 |
| | G | |
| HBG-mcrRNA-2 | mC*mU*mU*GACCAAUAGCCUUGACAGUUUGAGAGC | SEQ ID NO: |
| | UAUGCUGGGGCCCUGAAGAAGGGCCCUU*mU*mU*m | 17 |
| | G | |
| HBG-mcrRNA-3 | mC*mU*mU*GACCAAUAGCCUUGACAGUUUGAGAGC | SEQ ID NO: |
| | UAUGCUGUUUUGGGGCCCUGAAGAAGGG*mC*mC*m | 18 |
| | C | |
| HBG-mcrRNA-1- | mC*mU*mU*GACCAAUAGCCUUGACAGUUUGAGAGC | SEQ ID NO: |
| 2xboxB | UAGGGCCCUGAAGAAGGGCGGGCCCUGAAGAAGGG | 19 |
| | CCCAACCUGCUGUU*mU*mU*mG | |
| HBG-mcrRNA-O | mC*mU*mU*GACCAAUAGCCUUGACAGUUUGAGAGC | SEQ ID NO: |
| | UAUGCUGUU*mU*mU*mG | 20 |
| HBG-hcrRNA-O | mA*mC*mU*CCACCCAGUUUGAGAGCUAUGCUGUU* | SEQ ID NO: |
| | mU*mU*mG | 21 |
| tracrRNA-O, 3 | mG*mG*mA*ACCAUUCAAAACAGCAUAGCAAGUUCA | SEQ ID NO: |
| | AAUAAGGCUAGUCCGUUAUCAACUUGAAAAAGUGG | 22 |
| | CACCGAGUCGGUGCUUUU*mU*mU*mU | |
| tracrRNA-1 | mG*mG*mA*ACCAUUCAAAACAGCAAAAUAGCAAGU | SEQ ID NO: |
| | UCAAAUAAGGCUAGUCCGUUAUCAACUUGAAAAAG | 23 |
| | UGGCACCGAGUCGGUGCUUUU*mU*mU*mU | |
| tracrRNA-2 | mG*mG*mA*ACCAUUCAAAAAAACAGCAUAGCAAGU | SEQ ID NO: |
| | UCAAAUAAGGCUAGUCCGUUAUCAACUUGAAAAAG | 24 |
| | UGGCACCGAGUCGGUGCUUUU*mU*mU*mU | |
| TEV protease | MGESLFKGPRDYNPISSTICHLTNESDGHTTSLYGIGFGP | SEQ ID NO: |
| | FIITNKHLFRRNNGTLLVQSLHGVFKVKNTTTLQQHLID | 25 |
| | GRDMIIIRMPKDFPPFPQKLKFREPQREERICLVTTNFQT | |
| | KSMSSMVSDTSCTFPSSDGIFWKHWIQTKDGQCGSPLV | |
| | STRDGFIVGIHSASNFTNTNNYFTSVPKNFMELLTNQEA | |
| | QQWVSGWRLNADSVLWGGHKVFMVKPEEPFQPVKEA | |
| | TQLMN | |
| TEV protease N- | MGESLFKGPRDYNPISSTICHLTNESDGHTTSLYGIGFGP | SEQ ID NO: |
| terminal domain | FIITNKHLFRRNNGTLLVQSLHGVFKVKNTTTLQQHLID | 26 |
| | GRDMIIIRMPKDFPPFPQKLKFREPQREERICLVTTNFQT | |
| TEV protease C- | MKSMSSMVSDTSCTFPSSDGIFWKHWIQTKDGQCGSPL | SEQ ID NO: |
| terminal domain | VSTRDGFIVGIHSASNFTNTNNYFTSVPKNFMELLTNQE | 27 |
| | AQQWVSGWRLNADSVLWGGHKVFMVKPEEPFQPVKE | |
| | ATQ | |
| TEV protease | ENLYFQS | SEQ ID NO: |
| cleavage site | | 28 |
| TuMV protease | MASSNSMFRGLRDYNPISNNICHLTNVSDGASNSLYGV | SEQ ID NO: |
| | GFGPLILTNRHLFERNNGELVIKSRHGEFVIKNTTQLHL | 29 |
| | LPIPDRDLLLIRLPKDVPPFPQKLGFRQPEKGERICMVGS | |
| | NFQTKSITSIVSETSTIMPVENSQFWKHWISTKDGQCGS | |
| | PMVSTKDGKILGLHSLANFQNSINYFAAFPDDFAEKYL | |
| | HTIEAHEWVKHWKYNTSAISWGSLNIQASQPSGLFKVS | |
| | KLISDLDSTAVYAQ | |
| TuMV protease | GGCSHQS | SEQ ID NO: |
| cleavage site | | 30 |
| PPV protease | MASSKSLFRGLRDYNPIASSICQLNNSSGARQSEMFGLG | SEQ ID NO: |
| | FGGLIVTNQHLFKRNDGELTIRSHHGEFVVKDTKTLKL | 31 |
| | LPCKGRDIVIIRLPKDFPPFPRRLQFRTPTTEDRVCLIGSN | |
| | FQTKSISSTMSETSATYPVDNSHFWKHWISTKDGHCGL | |
| | PIVSTRDGSILGLHSLANSTNTQNFYAAFPDNFETTYLS | |
| | NQDNDNWIKQWRYNPDEVCWGSLQLKRDIPQSPFTIC | |
| | KLLTDLDGEFVYTQ | |
| PPV protease | QVVVHQSK | SEQ ID NO: |
| cleavage site | | 32 |
| PVY protease | MASAKSLMRGLRDFNPIAQTVCRLKVSVEYGASEMYG | SEQ ID NO: |
| | FGFGAYIVANHHLFRSYNGSMEVQSMHGTFRVKNLHS | 33 |
| | LSVLPIKGRDIILIKMPKDFPVFPQKLHFRAPTQNERICL | |
| | VGTNFQEKYASSIITETSTTYNIPGSTFWKHWIETDNGH | |
| | CGLPVVSTADGCIVGIHSLANNAHTTNYYSAFDEDFES | |
| | KYLRTNEHNEWVKSWVYNPDTVLWGPLKLKDSTPKG | |
| | LFKTTKLVQDLIDHDVVVEQ | |
| PVY protease | YDVRHQSR | SEQ ID NO: |
| cleavage site | | 34 |
| ZIKV protease | MASDMYIERAGDITWEKDAEVTGNSPRLDVALDESGD | SEQ ID NO: |
| | FSLVEEDGPPMREGGGGSGGGGSGALWDVPAPKEVKK | 35 |
| | GETTDGVYRVMTRRLLGSTQVGVGVMQEGVFHTMW | |
| | HVTKGAALRSGEGRLDPYWGDVKQDLVSYCGPWKLD | |
| | AAWDGLSEVQLLAVPPGERARNIQTLPGIFKTKDGDIG | |
| | AVALDYPAGTSGSPILDKCGRVIGLYGNGVVIKNGSYV | |
| | SAITQGKREEETPVECFE | |
| ZIKV protease | KERKRRGA | SEQ ID NO: |
| cleavage site | | 36 |
| WNV protease | MASSTDMWIERTADISWESDAEITGSSERVDVRLDDDG | SEQ ID NO: |
| | NFQLMNDPGAPWKGGGGSGGGGGVLWDTPSPKEYKK | 37 |
| | GDTTTGVYRIMTRGLLGSYQAGAGVMVEGVFHTLWH | |
| | TTKGAALMSGEGRLDPYWGSVKEDRLCYGGPWKLQH | |
| | KWNGQDEVQMIVVEPGKNVKNVQTKPGVFKTPEGEIG | |
| | AVTLDFPTGTSGSPIVDKNGDVIGLYGNGVIMPNGSYIS | |
| | AIVQGERMDEPIPAGFEPEML | |
| WNV protease | KQKKRGGK | SEQ ID NO: |
| cleavage site | | 38 |
| 2A peptide-1/ P2A | GSGATNFSLLKQAGDVEENPGP | SEQ ID NO: |
| | | 39 |
| 2A peptide-2/ T2A | GSGEGRGSLLTCGDVEENPGP | SEQ ID NO: |
| | | 40 |
| 2A peptide-3/ E2A | GSGQCTNYALLKLAGDVESNPGP | SEQ ID NO: |
| | | 41 |
| mouse APOBEC3 | MSSSTLSNICLTKGLPETRFWVEGRRMDPLSEEEFYSQF | SEQ ID NO: |
| cytidine deaminase | YNQRVKHLCYYHRMKPYLCYQLEQFNGQAPLKGCLLS | 42 |
| domain 2 (mA3- | EKGKQHAEILFLDKIRSMELSQVTITCYLTWSPCPNCA | |
| CDA2) | WQLAAFKRDRPDLILHIYTSRLYFHWKRPFQKGLCSLW | |
| | QSGILVDVMDLPQFTDCWTNFVNPKRPFWPWKGLEIIS | |
| | RRTQRRLRRIKESWGLQDLVNDFGNLQLGPPMS | |
| human | MNPQIRNPMERMYRDTFYDNFENEPILYGRSYTWLCY | SEQ ID NO: |
| APOBEC3B | EVKIKRGRSNLLWDTGVFRGQVYFKPQYHAEMCFLSW | 43 |
| cytidine deaminase | FCGNQLPAYKCFQITWFVSWTPCPDCVAKLAEFLSEHP | |
| domain 1 (hA3B- | NVTLTISAARLYYYWERDYRRALCRLSQAGARVKIMD | |
| CDA1) | YEEFAYCWENFVYNEGQ | |
| mouse APOBEC3 | MGPFCLGCSHRKCYSPIRNLISQETFKFHFKNLGYAKGR | SEQ ID NO: |
| cytidine deaminase | KDTFLCYEVTRKDCDSPVSLHHGVFKNKDNIHAEICFL | 44 |
| domain 1 (mA3- | YWFHDKVLKVLSPREEFKITWYMSWSPCFECAEQIVRF | |
| CDA1) | LATHHNLSLDIFSSRLYNVQDPETQQNLCRLVQEGAQV | |
| | AAMDLYEFKKCWKKFVDNGGRRFRPWKRLLTNFRYQ | |
| | DSKLQEILRPCYISVPSS | |
| human | MQFMPWYKFDENYAFLHRTLKEILRYLMDPDTFTFNF | SEQ ID NO: |
| APOBEC3B | NNDPLVLRRRQTYLCYEVERLDNGTWVLMDQHMGFL | 45 |
| cytidine deaminase | CNEAKNLLCGFYGRHAELRFLDLVPSLQLDPAQIYRVT | |
| domain 2 (hA3B- | WFISWSPCFSWGCAGEVRAFLQENTHVRLRIFAARIYD | |
| CDA2) | YDPLYKEALQMLRDAGAQVSIMTYDEFEYCWDTFVYR | |
| | QGCPFQPWDGLEEHSQALSGRLRAILQNQGN | |
| UGI | TNLSDIIEKETGKQLVIQESILMLPEEVEEVIGNKPESDIL | SEQ ID NO: |
| | VHTAYDES | 46 |
| | TDENVMLLTSDAPEYKPWALVIQDSNGENKIKML | |
| nCas9-D10A | MDKKYSIGLAIGTNSVGWAVITDEYKVPSKKFKVLGNT | SEQ ID NO: |
| | DRHSIKKNLIGALLFDSGETAEATRLKRTARRRYTRRK | 47 |
| | NRICYLQEIFSNEMAKVDDSFFHRLEESFLVEEDKKHER | |
| | HPIFGNIVDEVAYHEKYPTIYHLRKKLVDSTDKADLRLI | |
| | YLALAHMIKFRGHFLIEGDLNPDNSDVDKLFIQLVQTY | |
| | NQLFEENPINASGVDAKAILSARLSKSRRLENLIAQLPG | |
| | EKKNGLFGNLIALSLGLTPNFKSNFDLAEDAKLQLSKD | |
| | TYDDDLDNLLAQIGDQYADLFLAAKNLSDAILLSDILR | |
| | VNTEITKAPLSASMIKRYDEHHQDLTLLKALVRQQLPE | |
| | KYKEIFFDQSKNGYAGYIDGGASQEEFYKFIKPILEKMD | |
| | GTEELLVKLNREDLLRKQRTFDNGSIPHQIHLGELHAIL | |
| | RRQEDFYPFLKDNREKIEKILTFRIPYYVGPLARGNSRF | |
| | AWMTRKSEETITPWNFEEVVDKGASAQSFIERMTNFDK | |
| | NLPNEKVLPKHSLLYEYFTVYNELTKVKYVTEGMRKP | |
| | AFLSGEQKKAIVDLLFKTNRKVTVKQLKEDYFKKIECF | |
| | DSVEISGVEDRFNASLGTYHDLLKIIKDKDFLDNEENED | |
| | ILEDIVLTLTLFEDREMIEERLKTY AHLFDDKVMKQLKR | |
| | RRYTGWGRLSRKLINGIRDKQSGKTILDFLKSDGFANR | |
| | NFMQLIHDDSLTFKEDIQKAQVSGQGDSLHEHIANLAG | |
| | SPAIKKGILQTVKVVDELVKVMGRHKPENIVIEMAREN | |
| | QTTQKGQKNSRERMKRIEEGIKELGSQILKEHPVENTQL | |
| | QNEKLYLYYLQNGRDMYVDQELDINRLSDYDVDHIVP | |
| | QSFLKDDSIDNKVLTRSDKNRGKSDNVPSEEVVKKMK | |
| | NYWRQLLNAKLITQRKFDNLTKAERGGLSELDKAGFIK | |
| | RQLVETRQITKHVAQILDSRMNTKYDENDKLIREVKVI | |
| | TLKSKLVSDFRKDFQFYKVREINNYHHAHDAYLNAVV | |
| | GTALIKKYPKLESEFVYGDYKVYDVRKMIAKSEQEIGK | |
| | ATAKYFFYSNIMNFFKTEITLANGEIRKRPLIETNGETGE | |
| | IVWDKGRDFATVRKVLSMPQVNIVKKTEVQTGGFSKE | |
| | SILPKRNSDKLIARKKDWDPKKYGGFDSPTVAYSVLVV | |
| | AKVEKGKSKKLKSVKELLGITIMERSSFEKNPIDFLEAK | |
| | GYKEVKKDLIIKLPKYSLFELENGRKRMLASAGELQKG | |
| | NELALPSKYVNFLYLASHYEKLKGSPEDNEQKQLFVEQ | |
| | HKHYLDEIIEQISEFSKRVILADANLDKVLSAYNKHRDK | |
| | PIREQAENIIHLFTLTNLGAPAAFKYFDTTIDRKRYTSTK | |
| | EVLDATLIHQSITGLYETRIDLSQLGGD | |
| MS2 coat protein | MASNFTQFVLVDNGGTGDVTVAPSNFANGIAEWISSNS | SEQ ID NO: |
| (MCP) | RSQAYKVTCSVRQSSAQNRKYTIKVEVPKGAWRSYLN | 48 |
| | MELTIPIFATNSDCELIVKAMQGLLKDGNPIPSAIAANSG | |
| | IY | |
| PP7 coat protein | MGSKTIVLSVGEATRTLTEIQSTADRQIFEEKVGPLVGR | SEQ ID NO: |
| (PCP) | LRLTASLRQNGAKTAYRVNLKLDQADVVDSGLPKVRY | 49 |
| | TQVWSHDVTIVANSTEASRKSLYDLTKSLVATSQVEDL | |
| | VVNLVPLGR | |
| boxB coat protein | MGNARTRRRERRAEKQAQWKAAN | SEQ ID NO: |
| (N22p) | | 50 |
| Mouse APOBEC3 | SEKGKQHAEILFLDKIRSMELSQVTITCYLTWSPCPNCA | SEQ ID NO: |
| cytidine deaminase | WQLAAFKRDRPDLILHIYTSRLYFHWKRPFQKGLC | 51 |
| domain 2 core | | |
| (AA282-AA355) | | |
| Mus spicilegus A3 | SEKGKQHAEILFLDKIRSMELSQVTITCYLTWSPCPNCA | SEQ ID NO: |
| (AA248-AA321) | WQLAAFKRDRPDLIPHIYTSRLYFHWKRPFQKGLC | 52 |
| Cricetulus | SEKGKQHAEILFLDKIRSMELSQVTITCYLTWSPCPNCA | SEQ ID NO: |
| longicaudatus A3 | WRLAAFKRDRPDLILHIYTSRLYFHWKRPFQKGLC | 53 |
| (AA249-AA322) | | |
| Mus terricolor A3 | SEKGKQHAEILFLNKIRSMELSQVTITCYLTWSPCPNCA | SEQ ID NO: |
| (AA248-AA321) | WQLAAFKKDRPDLILHIYTSRLYFHWKRPFQKGLC | 54 |
| Mus caroli A3 | SKKGKQHAEILFLDKIRSMELSQVTITCYLTWSPCPNCA | SEQ ID NO: |
| (AA260-AA333) | WQLAAFKRDHPDLILHIYTSRLYFHWKRPFQKGLC | 55 |
| Mus pahari A3 | SKKGKQHAEILFLEKIRSMELSQMRITCYLTWSPCPNCA | SEQ ID NO: |
| (AA263-AA336) | WQLAAFQKDRPDLILHIYTSRLYFHWRRIFQKGLC | 56 |
| Mus shortridgei A3 | SKKGKQHAEILFLEKIRSMELSQMRITCYLTWSPCPNCA | SEQ ID NO: |
| (AA233-AA306) | WQLAAFQKDRPDLILHIYTSRLYFHWRRIFQKGLC | 57 |
| Mus setulosus A3 | SKKGKQHAEILFLDKIRSMELSQVRITCYLTWSPCPNCA | SEQ ID NO: |
| (AA29-AA302) | WQLETFKKDRPDLILHIYTSRLYFHWKRAFQEGLC | 58 |
| Grammomys | SKKGKPHAEILFLDKMWSMEELSQVRITCYLTWSPCPN | SEQ ID NO: |
| surdaster A3 | CARQLAAFKKDHPGLILRIYTSRLYFYWRRKFQKGLC | 59 |
| (AA270-AA344) | | |
| Rattus norvegicus | KKGEQHVEILFLEKMRSMELSQVRITCYLTWSPCPNCA | SEQ ID NO: |
| A3 (AA256- | RQLAAFKKDHPDLILRIYTSRLYFYWRKKFQKGLC | 60 |
| AA328) | | |
| Mastomys coucha | SKKGRQHAEILFLEKVRSMQLSQVRITCYLTWSPCPNC | SEQ ID NO: |
| A3 (AA258- | AWQLAAFKMDHPDLILRIYASRLYFHWRRAFQKGLC | 61 |
| AA331) | | |
| Cricetulus griseus | NKKGKHAEILFIDEMRSLELGQVQITCYLTWSPCPNCA | SEQ ID NO: |
| A3B (AA235- | QELAAFKSDHPDLVLRIYTSRLYFHWRRKYQEGLC | 62 |
| AA307) | | |
| Peromyscus | NKKGKHAEILFIDEMRSLELGQARITCYLTWSPCPNCA | SEQ ID NO: |
| leucopus A3 | QKLAAFKKDHPDLVLRVYTSRLYFHWRRKYQEGLC | 63 |
| (AA266-AA338) | | |
| Mesocricetus | NKKDKHAEILFIDKMRSLELCQVRITCYLTWSPCPNCA | SEQ ID NO: |
| auratus A3 | QELAAFKKDHPDLVLRIYTSRLYFHWRRKYQEGLC | 64 |
| (AA268-AA340) | | |
| Microtus | NKKGKHAEILFIDEMRSLKLSQERITCYLTWSPCPNCAQ | SEQ ID NO: |
| ochrogaster A3B | ELAAFKRDHPGLVLRIYASRLYFHWRRKYQEGLC | 65 |
| (AA266-AA338) | | |
| Nannospalax galili | NKRAKHAEILLIDMMRSMELGQVQITCYITWSPCPTCA | SEQ ID NO: |
| A3 (AA231- | QELAAFKQDHPDLVLRIYASRLYFHWKRKFQKGL | 66 |
| AA302) | | |
| Meriones | NKKGRHAEICLIDEMRSLGLGKAQITCYLTWSPCRKCA | SEQ ID NO: |
| unguiculatus A3 | QELATFKKDHPDLVLRVYASRLYFHWSRKYQQGLC | 67 |
| (AA233-AA305) | | |
| Dipodomys ordii | NKKGHHAEIRFIERIRSMGLDPSQDYQITCYLTWSPCLD | SEQ ID NO: |
| A3 (AA256- | CAFKLAKLKKDFPRLTLRIFTSRLYFHWIRKFQKGL | 68 |
| AA330) | | |
| Jaculus jaculus A3 | NKKGKHAEARFVDKMRSMQLDHALITCYLTWSPCLDC | SEQ ID NO: |
| (AA303-AA374) | SQKLAALKRDHPGLTLRIFTSRLYFHWVKKFQEGL | 69 |
| Chinchilla lanigera | SPQKGHHAESRFIKRISSMDLDRSRSYQITCFLTWSPCPS | SEQ ID NO: |
| A3H (AA86- | CAQELASFKRAHPHLRFQIFVSRLYFHWKRSYQAGL | 70 |
| AA161) | | |
| Heterocephalus | KKGYHAESRFIKRICSMDLGQDQSYQVTCFLTWSPCPH | SEQ ID NO: |
| glaber A3 (AA277- | CAQELVSFKRAHPHLRLQIFTARLFFHWKRSYQEGL | 71 |
| AA350) | | |
| Octodon degus A3 | KKGQHAEIRFIERIHSMALDQARSYQITCFLTWSPCPFC | SEQ ID NO: |
| (AA256-AA329) | AQELASFKSTHPRVHLQIFVSRLYFHWKRSYQEGL | 72 |
| Urocitellus parryii | NKKGHHAEIRFIKKIRSLDLDQSQNYEVTCYLTWSPCP | SEQ ID NO: |
| A3 (AA256- | DCAQELVALTRSHPHVRLRLFTSRLYFHWFWSFQEGL | 73 |
| AA330) | | |
| Aotus nancymaae | NRHAEICFIDEIESMGLDKTQCYEVTCYLTWSPCPSCAQ | SEQ ID NO: |
| A3H (AA75- | KLAAFTKAQVHLNLRIFASRLYYHWRSSYQKGL | 74 |
| AA146) | | |
| Cebus capucinus | NRHAEICFIDEIESMGLDKTQCYEVTCYLTWSPCPSCAQ | SEQ ID NO: |
| imitator A3H | KLVAFAKAQDHLNLRIFASRLYYHWRRRYKEGL | 75 |
| (AA55-AA126) | | |
| Saimiri boliviensis | HVEICFIDKIASMELDKTQCYDVTCYLTWSPCPSCAQK | SEQ ID NO: |
| boliviensis A3H | LAAFAKAQDHLNLRIFASRLYYHWRRSYQKGL | 76 |
| (AA56-AA125) | | |
| Homo sapiens A3H | NKKKCHAEICFINEIKSMGLDETQCYQVTCYLTWSPCS | SEQ ID NO: |
| (AA49-AA123) | SCAWELVDFIKAHDHLNLGIFASRLYYHWCKPQQKGL | 77 |
| Homo sapiens | ENKKKCHAEICFINEIKSMGLDETQCYQVTCYLTWSPC | SEQ ID NO: |
| ARP10 (AA48- | SSCAWELVDFIKAHDHLNLGIFASRLYYHWCKPQQKG | 78 |
| AA123) | L | |
| Pan paniscus A3H | NKKKCHAEICFINEIKSMGLDETQCYQVTCYLTWSPCS | SEQ ID NO: |
| (AA49-AA123) | SCAWKLVDFIQAHDHLNLRIFASRLYYHWCKPQQEGL | 79 |
| Symphalangus | NKKKRHAEIRFINKIKSMGLDETQCYQVTCYLTWSPCP | SEQ ID NO: |
| syndactylus A3H | SCAWELVDFIKAHDHLNLGIFASRLYYHWCRHQQEGL | 80 |
| (AA49-AA123) | | |
| Macaca mulatta | NKKKDHAEIRFINKIKSMGLDETQCYQVTCYLTWSPCP | SEQ ID NO: |
| A3H (AA49- | SCAGELVDFIKAHRHLNLRIFASRLYYHWRPNYQEGL | 81 |
| AA123) | | |
| Theropithecus | NKKKEHAEIRFINKIKSMGLDETQCYQVTCYLTWSPCP | SEQ ID NO: |
| gelada A3H | SCAGKLVDFIKAHHHLNLRIFASRLYYHWRPNYQEGL | 82 |
| (AA54-AA128) | | |
| Mandrillus | NKKKHHAEIHFINKIKSMGLDETQCYQVTCYLTWSPCP | SEQ ID NO: |
| leucophaeus A3H | SCARELVDFIKAHRHLNLRIFASRLYYHWRPHYQEGL | 83 |
| (AA49-AA123) | | |
| Bos grunniens A3 | NKKQRHAEIRFIDKINSLDLNPSQSYKIICYITWSPCPNC | SEQ ID NO: |
| (AA74-AA148) | ANELVNFITRNNHLKLEIFASRLYFHWIKPFKMGL | 84 |
| Bubalus bubalis A3 | NKKQRHAEIRFIDKINSLDLNPSQSYKIICYITWSPCPNC | SEQ ID NO: |
| (AA74-AA148) | ASELVDFITRNDHLDLQIFASRLYFHWIKPFKRGL | 85 |
| Odocoileus | NKKQRHAEIRFIDKINSLNLDRRQSYKIICYITWSPCPRC | SEQ ID NO: |
| virginianus texanus | ASELVDFITGNDHLNLQIFASRLYFHWKKPFQRGL | 86 |
| A3H (AA209- | | |
| AA283) | | |
| Sus scrofa A3 | NKKKRHAEIRFIDKINSLNLDQNQCYRIICYVTWSPCHN | SEQ ID NO: |
| (AA51-AA125) | CAKELVDFISNRHHLSLQLFASRLYFHWVRCYQRGL | 87 |
| Ceratotherium | NKKKRHAEIRFIDKIKSLGLDRVQSYEITCYITWSPCPTC | SEQ ID NO: |
| simum simum A3B | ALELVAFTRDYPRLSLQIFASRLYFHWRRRSIQGL | 88 |
| (AA232-AA306) | | |
| Equus caballus | NKKKRHAEIRFIDKINSLGLDQDQSYEITCYVTWSPCAT | SEQ ID NO: |
| A3H (AA79- | CACKLIKFTRKFPNLSLRIFVSRLYYHWFRQNQQGL | 89 |
| AA153) | | |
| Enhydra lutris | KKKRHAEIRFIDSIRALQLDQSQRFEITCYLTWSPCPTCA | SEQ ID NO: |
| kenyoni A3B | KELAMFVQDHPHISLRLFASRLYFHWRWKYQEGL | 90 |
| (AA243-AA316) | | |
| Leptonychotes | KKKRHAEIRFIDNIKALRLDTSQRFEITCYVTWSPCPTC | SEQ ID NO: |
| weddellii A3H | AKELVAFVRDHRHISLRLFASRLYFHWLRENKKGL | 91 |
| (AA50-AA123) | | |
| Ursus arctos | NKKKRHAEIRFIDKIRSLORDSSQTFEITCYVTWSPCFTC | SEQ ID NO: |
| horribilis A3F | AEELVAFVRDHPHVRLRLFASRLYFHWLRKYQEGL | 92 |
| (AA552-AA626) | | |
| Panthera leo | NKKKRHAEICFIDKIKSLTRDTSQRFEIICYITWSPCPFCA | SEQ ID NO: |
| bleyenberghi A3H | EELVAFVKDNPHLSLRIFASRLYVHWRWKYQQGL | 93 |
| (AA50-AA124) | | |
| Panthera tigris | NKKKRHAEICFIDKIKSLTRDTSQRFEIICYITWSPCPFCA | SEQ ID NO: |
| sumatrae A3H | EELVAFVKDNPHLSLRIFASRLYVHWRWKYQQGL | 94 |
| (AA50-AA124) | | |
| Tupaia belangeri | NKKHRHAEVRFIAKIRSMSLDLDQKHQLTCYLTWSPCP | SEQ ID NO: |
| A3 (AA46-AA120) | SCAQELVTFMAESRHLNLQVFVSRLYFHWQRDFQQGL | 95 |
| Gorilla A3B | GRSYNWLCYEVKIKRGRSNLLWNTGVFRGQMYSQPEH | SEQ ID NO: |
| (AA29-AA138) | HAEMCFLSWFCGNQLPAYKCFQITWFVSWTPCPDCVA | 96 |
| | KLAEFLAEYPNVTLTISTARLYYYWERDYRRALCRL | |
| Pan paniscus A3B | GRSYTWLCYEVKIRRGHSNLLWDTGVFRGQMYSQPEH | SEQ ID NO: |
| (AA29-AA138) | HAEMYFLSWFCGNQLPAYKCFQITWFVSWTPCPDCVA | 97 |
| | KLAEFLAEHPNVTLTISAARLYYYWERDYRRALCRL | |
| Pan troglodytes | GRSYTWLCYEVKIRRGHSNLLWDTGVFRGQMYSQPEH | SEQ ID NO: |
| A3B (AA29- | HAEMCFLSWFCGNQLSAYKCFQITWFVSWTPCPDCVA | 98 |
| AA138) | KLAKFLAEHPNVTLTISAARLYYYWERDYRRALCRL | |
| Gorilla A3F | RNTVWLCYEVKTKGPSRPPLDAKIFRGQVYFEPQYHAE | SEQ ID NO: |
| (AA30-AA137) | MCFLSWFCGNQLPAYKCFQITWFVSWTPCPDCVAKLA | 99 |
| | EFLAEHPNVTLTISAARLYYYWE | |
| Pan troglodytes | RNTVWLCYEVKTKGPSRPRLDTKIFRGQVYFEPQYHAE | SEQ ID NO: |
| A3F (AA30- | MCFLSWFCGNQLPAYKCFQITWFVSWTPCPDCVAKLA | 100 |
| AA137) | EFLAEHPNVTLTISAARLYYYWERDYRRALCRL | |
| Human sapiens | RNTVWLCYEVKTKGPSRPRLDAKIFRGQVYSQPEHHA | SEQ ID NO: |
| A3F (AA30- | EMCFLSWFCGNQLPAYKCFQITWFVSWTPCPDCVAKL | 101 |
| AA137) | AEFLAEHPNVTLTISAARLYYYWERDYRRALCRL | |
| Macaca leonine | RNTVWLCYEVKTRGPSMPTWGTKIFRGQVCFEPQYHA | SEQ ID NO: |
| A3F (AA30- | EMCFLSRFCGNQLPAYKRFQITWFVSWTPCPDCVAKV | 102 |
| AA137) | AEFLAEHPNVTLTISAARLYYYWETDYRRALCRL | |
| Macaca nemestrina | RNTVWLCYEVKTRGPSMPTWGTKIFRGQVCFEPQYHA | SEQ ID NO: |
| A3F (AA30- | EMCFLSRFCGNQLPAYKRFQITWFVSWTPCPDCVAKV | 103 |
| AA137) | AEFLAEHPNVTLTISAARLYYYWETDYRRALCRL | |
| Rhinopithecus | RNTVWLCYEVKTRGPSMPTWGAKIFRGQVYFEPQYHA | SEQ ID NO: |
| roxellana A3F | EMCFLSWFCGNQLPAYKRFQITWFVSWTPCPDCVAKV | 104 |
| (AA30-AA137) | AEFLAEHPNVTLTISAARLYYYWETDYRRALCRL | |
| Mandrillus | RNTVWLCYKVKTRGPSMPTWGTKIFRGQVYFQPQYHA | SEQ ID NO: |
| leucophaeus A3F | EMCFLSWFCGNQLPAYKRFQITWFVSWTPCPDCVVKV | 105 |
| (AA30-AA130) | AEFLAEHPNVTLTISAARLYYYWETDY | |
| Macaca mulatta | RNTVWLCYEVKTRGPSMPTWDTKIFRGQVYSKPEHHA | SEQ ID NO: |
| A3F (AA30- | EMCFLSRFCGNQLPAYKRFQITWFVSWTPCPDCVAKV | 106 |
| AA137) | AEFLAEHPNVTLTISAARLYYYWETDYRRALCRL | |
| Theropithecus | RNTVWLCYEVKTRGPSMPTWGTKIFRGQVYFQPQYHA | SEQ ID NO: |
| gelada A3F | EMCFLSRFCGNQLPAYKRFQITWFVSWNPCPDCVAKVI | 107 |
| (AA30-AA137) | EFLAEHPNVTLTISAARLYYYWGRDWRRALRRL | |
| Cercocebus atys | GRSYTWLCYEVKIRKDPSKLPWYTGVFRGQVYSKPEH | SEQ ID NO: |
| A3B (AA29- | HAEMCFLSRFCGNQLPAYKRFQITWFVSWNPCPDCVA | 108 |
| AA138) | KVIEFLAEHPNVTLTISAARLYYYWSRDWQRALCRL | |
| Macaca fascicularis | GRSYTWLCYEVKIRKDPSKLPWDTGVFRGQVYSKPEH | SEQ ID NO: |
| A3B (AA29- | HAEMCFLSRFCGNQLPAYKRFQITWFVSWNPCPDCVA | 109 |
| AA138) | KVIEFLAEHPNVTLTISTARLYYYWGRDWQRALCRL | |
| Macaca mulatta | GRSYTWLCYEVKIRKDPSKLPWDTGVFRGQVYSKPEH | SEQ ID NO: |
| A3B (AA29- | HAEMCFLSRFCGNQLPAYKRFQITWFVSWNPCPDCVA | 110 |
| AA138) | KVIEFLAEHPNVTLTISTARLYYYWGRDWQRALCRL | |
| Macaca leonina | GRSYTWLCYEVKIRKDPSKLPWDTGVFRGQVYSKPEH | SEQ ID NO: |
| A3B (AA29- | HAEMCFLSRFCGNQLPAYKRFQITWFVSWNPCPDCVV | 111 |
| AA138) | KVIEFLAEHPNVTLTISTARLYYYWGRDWQRALCRL | |
| Mandrillus | GRSYTWLCYEVKIRKDPSKLPWYTGVFRGQVYSKPEH | SEQ ID NO: |
| leucophaeus A3B | HAEMCFLSRFCGNQLPAYKRFQITWFVSWNPCPDCVA | 112 |
| (AA29-AA138) | KVIEFLAEHPNVTLTIFTARLYYYWGRDWQRALCRL | |
| Macaca nemestrina | GRSYTWLCYEVKIRKDPSKLPWDTGVFRGQVYSKPEH | SEQ ID NO: |
| A3B (AA29- | HAEMCFLSRFCGNQLPAYKRFQITWFVSWNPCPDCVA | 113 |
| AA138) | KVTEFLAEHPNVTLTISTARLYYYWGRDWQRALCRL | |
| Rhinopithecus bieti | GRSYTWLCYEVKIRKDPSKLPWDTGVFRGQVYSEPEH | SEQ ID NO: |
| A3F (AA29- | HAEMYFLSWFCGNQLPAYKRFQITWFVSWTPCPDCVA | 114 |
| AA138) | KVAEFLTEHPNVTLTISAARLYYYRGRDWRRALCRL | |
| Rhinopithecus | GRSYTWLCYEVKIRKDPSKLPWDTGVFRGQVYSEPEH | SEQ ID NO: |
| roxellana A3B | HAEMYFLSWFCGNQLPAYKRFQITWFVSWTPCPDCVA | 115 |
| (AA29-AA138) | KVAEFLTEHPNVTLTISAARLYYYRGRDWRRALCRL | |
| Chlorocebus | GRSYTWLCYEVKIRKDPSKLPWDTGVFRGQMYSKPEH | SEQ ID NO: |
| sabaeus A3B | HAEMCFLSWFCGNQLPAHKRFQITWFVSWTPCPDCVA | 116 |
| (AA29-AA138) | KVAEFLAEYPNVTLTISAARLYYYWETDYRRALCRL | |
| Nomascus | RSYTWLCYEVKIRKDPSKLPWDTGVFRGQMYFQPEYH | SEQ ID NO: |
| leucogenys A3B | AEMCFLSWFCGNQLPAYKRFQITWFVSWTPCPDCVAK | 117 |
| (AA30-AA138) | VAVFLAEHPNVTLTISAARLYYYWEKDWQRALCRL | |
| Cercocebus atys | GRSYTWLCYEVKIKKYPSKLLWDTGVFQGQVYFQPQY | SEQ ID NO: |
| A3F (AA29- | HAEMCFLSRFCGNQLPAYKRFQITWFVSWNPCPDCVA | 118 |
| AA138) | KVTEFLAEHPNVTLTISAARLYYYWEKDXRRALRRL | |
| Papio anubis A3F | GRSYTWLCYEVKIKEDPSKLLWDTGVFQGQVYFQPQY | SEQ ID NO: |
| (AA29-AA138) | HAEMCFLSRFCGNQLPAYKRFQITWFVSWNPCPDCVA | 119 |
| | KVTEFLAEHPNVTLTISAARLYYYWGRDWRRALRRL | |
| Chlorocebus | GRRYTWLCYEVKIKKDPSKLPWDTGVFPGQVRPKFQS | SEQ ID NO: |
| aethiops A3D | NRRYEVYFQPQYHAEMYFLSWFCGNQLPAYKHFQITW | 120 |
| (AA29-AA150) | FVSWNPCPDCVAKVTEFLAEHRNVTLTISAARLYYYW | |
| | GKDWRRALCRL | |
| Chlorocebus | GRRYTWLCYEVKIKKDPSKLPWDTGVFPGQPQYHAEM | SEQ ID NO: |
| sabaeus A3D | YFLSWFCGNQLPAYKHFQITWFVSWNPCPDCVAKVTE | 121 |
| (AA29-AA134) | FLAEHRNVTLTISAARLYYYWGKDWRRALCRL | |
| Chlorocebus | GRRYTWLCYEVKIKKDPSKLPWDTGVFPGQVRPKFQS | SEQ ID NO: |
| sabaeus A3F | NRRQKVYFQPQYHAEMYFLSWFCGNQLPAYKHFQITW | 122 |
| (AA29-AA150) | FVSWNPCPDCVAKVTEFLAEHRNVTLTISAARLYYYW | |
| | GKDWRRALCRL | |
| Erythrocebus patas | GRRYTWLCYEVKIKKDPSKLPWDTGVFQGQVRPKFQS | SEQ ID NO: |
| A3D (AA29- | NRRYEVYFQPQYHAEMCFLSWFCGNQLPAYKHFQITW | 123 |
| AA150) | FVSWNPCPDCVAKVTEFLAEHPNVTLTISAARLYYYW | |
| | GKDWRRALCRL | |
| Macaca fascicularis | GRSYTWLCYEVKIRKDPSKLPWDTGVFRGQVRPKLQS | SEQ ID NO: |
| A3D (AA29- | NRRYELSNWECRKRVYFQPQYHAEMYFLSWFCGNQLP | 124 |
| AA159) | ANKRFQITWFASWNPCPDCVAKVTEFLAEHPNVTLTIS | |
| | VARLYYYRGKDWRRALRRL | |
| Macaca fascicularis | GRSYTWLCYEVKIRKDPSKLPWDTGVFRGQVYFQPQY | SEQ ID NO: |
| A3F (AA29- | HAEMYFLSWFCGNQLPANKRFQITWFASWNPCPDCVA | 125 |
| AA138) | KVTEFLAEHPNVTLTISVARLYYYRGKDWRRALRRL | |
| Macaca nemestrina | GRSYTWLCYEVKIRKDPSKLPWDTGVFRDQVYFQPQY | SEQ ID NO: |
| A3D (AA29- | HAEMCFLSWFCGNQLPANKRFQITWFVSWNPCPDCVT | 126 |
| AA138) | KVTEFLAEHPNVTLTISVARLYYYRGKDWRRALRRL | |
| Macaca leonina | GRSYTWLCYEVKIRKDPSKLPWYTGVFRGQVYFQPQY | SEQ ID NO: |
| A3D (AA29- | HAEMCFLSWFCGNQLPANKRFQITWFVSWNPCPDCVA | 127 |
| AA138) | KVTEFLAEHPNVTLTISVARLYYYRGKDWRRALRRL | |
| Macaca mulatta | GRSYTWLCYEVKIRKDPSKLPWDTGVFRGQVYFQPQY | SEQ ID NO: |
| A3D (AA29- | HAEMCFLSWFCGNQLPAYKRFQITWFVSWNPCPDCVA | 128 |
| AA138) | KVTEFLAEHPNVTLTISVARLYYYRGKDWRRALCRL | |
| Gorilla A3D | GRSYTWLCYEVKIRRGSSNLLWNTGVFRGPVPPKLQSN | SEQ ID NO: |
| (AA29-AA150) | HRQEVYFQFENHAEMCFLSWFCGNRLPANRRFQITWF | 129 |
| | VSWNPCLPCVVKVTKFLAEHPNVTLTISAARLYYYRDR | |
| | EWRRVLRRL | |
| Pan paniscus A3D | GRSYTWLCYEVKIKRGCSNLIWDTGVFRGPVLPKLQSN | SEQ ID NO: |
| (AA29-AA150) | HRQEVYFQFENHAEMCFFSWFCGNRLPANRRFQITWF | 130 |
| | VSWNPCLPCVVKVTKFLAEHPNVTLTISAARLYYYQDR | |
| | EWRRVLRRL | |
| Pan troglodytes | GRSYTWLCYEVKIKRGCSNLIWDTGVFRGPVLPKLQSN | SEQ ID NO: |
| A3D (AA29- | HRQEVYFQFENHAEMCFFSWFCGNRLPANRRFQITWF | 131 |
| AA150) | VSWNPCLPCVVKVTKFLAEHPNVTLTISAARLYYYQDR | |
| | EWRRVLRRL | |
| Homo sapiens A3D | GRSYTWLCYEVKIKRGRSNLLWDTGVFRGPVLPKRQS | SEQ ID NO: |
| (AA29-AA150) | NHRQEVYFRFENHAEMCFLSWFCGNRLPANRRFQITW | 132 |
| | FVSWNPCLPCVVKVTKFLAEHPNVTLTISAARLYYYRD | |
| | RDWRWVLLRL | |
| Nomascus | GRSYTWLCYEVKIRKDPSKLPWDKGVFRGQVLPKFQS | SEQ ID NO: |
| leucogenys A3D | NHRQEVYFQLENHAEMCFLSWFCGNQLPANRRFQITW | 133 |
| (AA29-AA150) | FVSWNPCLPCVAKVTEFLAEHPNVTLTISAARLYYYRG | |
| | RDWRRALRRL | |
| Saimiri boliviensis | GKKYTWLCYEVKIKKDTSKLPWNTGVFRGQVNFNPEH | SEQ ID NO: |
| A3C (AA29- | HAEMYFLSWFRGKLLPACKRSQITWFVSWNPCLYCVA | 134 |
| AA138) | KVAEFLAEHPNVTLTVSTARLYCYWKKDWRRALRKL | |
| Saimiri boliviensis | GKKYTWLCYEVKIKKDTSKLPWNTGVFRGQVNFNPEH | SEQ ID NO: |
| A3F (AA29- | HAEMYFLSWFRGKLLPACKRSQITWFVSWNPCLYCVA | 135 |
| AA138) | KVAEFLAEHPNVTLTVSTARLYCYWKKDWRRALRKL | |
| Piliocolobus | GRRYTWLCYEVKIMKDHSKLPWYTGVFRGQVYFEPQ | SEQ ID NO: |
| tephrosceles A3F | NHAEMCFLSWFCGNQLPAYECCQITWFVSWTPCPDCV | 136 |
| (AA36-AA145) | AKVTEFLAEHPNVTLTISAARLYYYRGRDWRRALRRL | |
| Colobus angolensis | GRRYTWLCYEVKISKDPSKLPWDTGIFRGQVYFEPQYH | SEQ ID NO: |
| palliatus A3F | AEMCFLSWYCGNQLPAYKCFQITWFVSWTPCPDCVGK | 137 |
| (AA29-AA138) | VAEFLAEHPNVTLTISAARLYYYWETDYRRALCRL | |
| Pongo abelii A3F | RNYTWLCYEVKIRKDPSKLAWDTGVFRGQVLPKLQSN | SEQ ID NO: |
| (AA30-AA150) | HRREVYFEPQYHAEMCFLSWFCGNQLSAYERFQITWF | 138 |
| | VSWTPCPDCVAMLAEFLAEHPNVTLTVSAARLYYYWE | |
| | RDYRGALRRL | |
| MS2 | ACAUGAGGAUCACCCAUGU | SEQ ID NO: |
| | | 139 |
| PP7 | GGAGCAGACGAUAUGGCGUCGCUCC | SEQ ID NO: |
| | | 140 |
| boxB | GCCCUGAAGAAGGGC | SEQ ID NO: |
| | | 141 |
| Telomerase Ku | UUGUGUUUCUACUUAUAGAUGGCUAAAAUCUGAGU | SEQ ID NO: |
| binding motif | UUAGAAAAUGCAA | 142 |
| Telomerase Sm7 | AAUUUUUGGA | SEQ ID NO: |
| binding motif | | 143 |
| SfMu | CUGAAUGCCUGCGAGCAUC | SEQ ID NO: |
| | | 144 |
| KU70 | MELDPDDVFRDEDEDPENDFFQEKEASKEFVVYLIDAS | SEQ ID NO: |
| | PKMFCSTCPSEEEDKQESHFHIAVSCIAQSLKAHIINRSN | 145 |
| | DEIAICFFNTREKKNLQDLNGVYVFNVPERDSIDRPTAR | |
| | LIKEFDLIEESFDKEIGSQTGIVSDSRENSLYSALWVAQA | |
| | LLRKGSLKTADKRMFLFTNEDDPFGSMRISVKEDMTRT | |
| | TLQRAKDAQDLGISIELLPLSQPDKQFNITLFYKDLIGLN | |
| | SDELTEFMPSVGQKLEDMKDQLKKRVLAKRIAKRITFV | |
| | ICDGLSIELNGYALLRPAIPGSITWLDSTTNLPVKVERSY | |
| | ICTDTGAIMQDPIQRIQPYKNQNIMFTVEELSQVKRISTG | |
| | HLRLLGFKPLSCLKDYHNLKPSTFLYPSDKEVIGSTRAFI | |
| | ALHRSMIQLERFAVAFYGGTTPPRLVALVAQDEIESDG | |
| | GQVEPPGINMIYLPYANDIRDIDELHSKPGVAAPRASDD | |
| | QLKKASALMRRLELKDFSVCQFANPALQRHYAILQAIA | |
| | LDENELRETRDETLPDEEGMNRPAVVKAIEQFKQSIYG | |
| | DDPDEESDSGAKEKSKKRKAGDADDGKYDYIELAKTG | |
| | KLKDLTVVELKTYLTANNLLVSGKKEVLINRILTHIGK | |
| KU80 | MSSESTTFIVDVSPSMMKNNNVSKSMAYLEYTLLNKSK | SEQ ID NO: |
| | KSRKTDWISCYLANCPVSENSQEIPNVFQIQSFLAPVTT | 146 |
| | TATIGFIKRLKQYCDQHSHDSSNEGLQSMIQCLLVVSLD | |
| | IKQQFQARKILKQIVVFTDNLDDLDITDEEIDLLTEELST | |
| | RIILIDCGKDTQEERKKSNWLKLVEAIPNSRIYNMNELL | |
| | VEITSPATSVVKPVRVFSGELRLGADILSTQTSNPSGSM | |
| | QDENCLCIKVEAFPATKAVSGLNRKTAVEVEDSQKKER | |
| | YVGVKSIIEYEIHNEGNKKNVSEDDQSGSSYIPVTISKDS | |
| | VTKAYRYGADYVVLPSVLVDQTVYESFPGLDLRGFLN | |
| | REALPRYFLTSESSFITADTRLGCQSDLMAFSALVDVML | |
| | ENRKIAVARYVSKKDSEVNMCALCPVLIEHSNINSEKK | |
| | FVKSLTLCRLPFAEDERVTDFPKLLDRTTTSGVPLKKET | |
| | DGHQIDELMEQFVDSMDTDELPEIPLGNYYQPIGEVTT | |
| | DTTLPLPSLNKDQEENKKDPLRIPTVFVYRQQQVLLEWI | |
| | HQLMINDSREFEIPELPDSLKNKISPYTHKKFDSTKLVE | |
| | VLGIKKVDKLKLDSELKTELEREKIPDLETLLKRGEQHS | |
| | RGSPNNSNN | |
| Sm7-like protein | GSVIDVSSQRVNVQRPLDALGNSLNSPVIIKLKGDREFR | SEQ ID NO: |
| | GVLKSFDLHMNLVLNDAEELEDGEVTRRLGT | 147 |
| SfMu Com binding | MKSIRCKNCNKLLFKADSFDHIEIRCPRCKRHIIMLNAC | SEQ ID NO: |
| protein | EHPTEKHCGKREKITHSDETVRY | 148 |
| hcrRNA-3U | GUUUGAGAGCUAUGCUGUUUUGGGCCAACAUGAGG | SEQ ID NO. |
| | AUCACCCAUGUCUGCAGGGCCUUU | 149 |
| hcrRNA-4U | GUUUGAGAGCUAUGCUGUUUUGGGCCAGCGAGGAG | SEQ ID NO. |
| | CACCAGCCCUCGCGUGUGACGAUGACGAUCACGCA | 150 |
| | UCGUUU | |
| hcrRNA-5U | GUUUGAGAGCUAUGCUGUUUUGGGCCAGGAAUGAC | SEQ ID NO. |
| | CACCAGGCAUUCCGAUCCGACGAUGGACCAUCAGG | 151 |
| | CCAUCGUUU | |
| mcrRNA-3U | GUUUGAGAGCUAUGCUGUUUUGGGGCCCUGAAGAA | SEQ ID NO. |
| | GGGCCCUUU | 152 |
| mcrRNA-4U | GUUUGAGAGCUAUGCUGUUUUGGGGCCCUGAAGAA | SEQ ID NO. |
| | GGGCCCGGGCCCUGAAGAAGGGCCCUUU | 153 |
| HBG-hcrRNA-3U | mA*mC*mU*CCACCCAGUUUGAGAGCUAUGCUGUUU | SEQ ID NO. |
| | UGGGCCAACAUGAGGAUCACCCAUGUCUGCAGGGC | 154 |
| | C*mU*mU*mU | |
| HBG-hcrRNA-4U | mA*mC*mU*CCACCCAGUUUGAGAGCUAUGCUGUUU | SEQ ID NO. |
| | UGGGCCAGCGAGGAGCACCAGCCCUCGCGUGUGAC | 155 |
| | GAUGACGAUCACGCAUCG*mU*mU*mU | |
| HBG-hcrRNA-5U | mA*mC*mU*CCACCCAGUUUGAGAGCUAUGCUGUUU | SEQ ID NO. |
| | UGGGCCAGGAAUGACCACCAGGCAUUCCGAUCCGA | 156 |
| | CGAUGGACCAUCAGGCCAUCG*mU*mU*mU | |
| HBG-mcrRNA-3U | mC*mU*mU*GACCAAUAGCCUUGACAGUUUGAGAGC | SEQ ID NO. |
| | UAUGCUGUUUUGGGGCCCUGAAGAAGGGCCC*mU* | 157 |
| | mU*mU | |
| HBG-mcrRNA-4U | mC*mU*mU*GACCAAUAGCCUUGACAGUUUGAGAGC | SEQ ID NO. |
| | UAUGCUGUUUUGGGGCCCUGAAGAAGGGCCCGGGC | 158 |
| | CCUGAAGAAGGGCCC*mU*mU*mU | |
| hcrRNA-1 | NNNNNNNNNNGUUUGAGAGCUAGGCCAACAUGAGG | SEQ ID NO. |
| | AUCACCCAUGUCUGCAGGGCCUGCUGUUUUG | 288 |
| hcrRNA-2 | NNNNNNNNNNGUUUGAGAGCUAUGCUGGGCCAACA | SEQ ID NO. |
| | UGAGGAUCACCCAUGUCUGCAGGGCCUUUUG | 289 |
| hcrRNA-3 | NNNNNNNNNNGUUUGAGAGCUAUGCUGUUUUGGGC | SEQ ID NO. |
| | CAACAUGAGGAUCACCCAUGUCUGCAGGGCC | 290 |
| mcrRNA-1 | NNNNNNNNNNNNNNNNNNNNGUUUGAGAGCUAGG | SEQ ID NO. |
| | GCCCUGAAGAAGGGCCCUGCUGUUUUG | 291 |
| mcrRNA-2 | NNNNNNNNNNNNNNNNNNNNGUUUGAGAGCUAUGC | SEQ ID NO. |
| | UGGGGCCCUGAAGAAGGGCCCUUUUG | 292 |
| mcrRNA-3 | NNNNNNNNNNNNNNNNNNNNGUUUGAGAGCUAUGC | SEQ ID NO. |
| | UGUUUUGGGGCCCUGAAGAAGGGCCC | 293 |
| mcrRNA-1- | NNNNNNNNNNNNNNNNNNNNGUUUGAGAGCUAGG | SEQ ID NO. |
| 2xboxB | GCCCUGAAGAAGGGCGGGCCCUGAAGAAGGGCCCA | 294 |
| | ACCUGCUGUUUUG | |
| mcrRNA-O | NNNNNNNNNNNNNNNNNNNNGUUUGAGAGCUAUGC | SEQ ID NO. |
| | UGUUUUG | 295 |
| hcrRNA-O | NNNNNNNNNNGUUUGAGAGCUAUGCUGUUUUG | SEQ ID NO. |
| | | 296 |
| hcrRNA-3U | NNNNNNNNNNGUUUGAGAGCUAUGCUGUUUUGGGC | SEQ ID NO. |
| | CAACAUGAGGAUCACCCAUGUCUGCAGGGCCUUU | 297 |
| hcrRNA-4U | NNNNNNNNNNGUUUGAGAGCUAUGCUGUUUUGGGC | SEQ ID NO. |
| | CAGCGAGGAGCACCAGCCCUCGCGUGUGACGAUGA | 298 |
| | CGAUCACGCAUCGUUU | |
| hcrRNA-5U | NNNNNNNNNNGUUUGAGAGCUAUGCUGUUUUGGGC | SEQ ID NO. |
| | CAGGAAUGACCACCAGGCAUUCCGAUCCGACGAUG | 299 |
| | GACCAUCAGGCCAUCGUUU | |
| mcrRNA-3U | NNNNNNNNNNNNNNNNNNNNGUUUGAGAGCUAUGC | SEQ ID NO. |
| | UGUUUUGGGGCCCUGAAGAAGGGCCCUUU | 300 |
| mcrRNA-4U | NNNNNNNNNNNNNNNNNNNNGUUUGAGAGCUAUGC | SEQ ID NO. |
| | UGUUUUGGGGCCCUGAAGAAGGGCCCGGGCCCUGA | 301 |
| | AGAAGGGCCCUUU | |
| hcr-PCSK9-Mouse | mU*mU*mC*CUCUGUCGUUUGAGAGCUAUGCUGUUU | SEQ ID NO. |
| | UGGGCCAACAUGAGGAUCACCCAUGUCUGCAGGGC | 302 |
| | C*mU*mU*mU | |
| mcr-PCSK9-Mouse | mC*mA*mG*GUUCCAUGGGAUGCUCUGUUUGAGAGC | SEQ ID NO. |
| | UAUGCUGUUUUGGGGCCCUGAAGAAGGGCCC*mU* | 303 |
| | mU*mU | |
| hcr-PCSK9-Human | mU*mU*mC*AUCCGCCGUUUGAGAGCUAUGCUGUUU | SEQ ID NO. |
| | UGGGCCAACAUGAGGAUCACCCAUGUCUGCAGGGC | 304 |
| | C*mU*mU*mU | |
| mcr-PCSK9- | mC*mA*mG*GUUCCACGGGAUGCUCUGUUUGAGAGC | SEQ ID NO. |
| Human | UAUGCUGUUUUGGGGCCCUGAAGAAGGGCCC*mU* | 305 |
| | mU*mU | |
| hcr-PCSK9- | mU*mU*mC*AUCCGCCGUUUGAGAGCUAUGCUGUUU | SEQ ID NO. |
| Monkey | UGGGCCAACAUGAGGAUCACCCAUGUCUGCAGGGC | 306 |
| | C*mU*mU*mU | |
| mcr-PCSK9- | mC*mA*mG*GUUCCAUGGGAUGCUCUGUUUGAGAGC | SEQ ID NO. |
| Monkey | UAUGCUGUUUUGGGGCCCUGAAGAAGGGCCC*mU* | 307 |
| | mU*mU | |
| tBE-V5-mA3 | gggggacagatcgcctggagacgccatccacgctgttttgacctccatagaagacacc | SEQ ID NO. |
| mRNA | gggaccgatccagcctccgcggccgggaacggtgcattggaacgcggattccccgtg | 360 |
| | ccaagagtgactcaccgtccttgacacggccaccatggcctctaacttcacccagtttgt | |
| | gctggtggacaatggaggaaccggcgatgtgacagtggcaccatccaactttgccaat | |
| | ggcatcgccgagtggatcagctccaactctaggagccaggcctacaaggtgacctgc | |
| | agcgtgcggcagtctagcgcccagaatagaaagtacacaatcaaggtggaggtgcca | |
| | aagggagcatggcgcagctatctgaacatggagctgaccatccccatcttcgccacaa | |
| | attccgactgtgagctgatcgtgaaggccatgcagggcctgctgaaggatggcaaccc | |
| | catcccttctgccatogccgccaatagcggcatctactctggaggatctagcggaggca | |
| | gctctggcagcgagacaccaggaacaagcgagtcagcaacaccagagagcagtgg | |
| | cggcagcagcggcggcagcageggoggatccaagcggcccgccgccaccaagaa | |
| | ggccggccaggccaagaagaagaagatgaccaacctgtctgacatcatcgagaagg | |
| | agacaggcaagcagctggtcatccaggagagcatcctgatgctgcccgaagaagtcg | |
| | aagaagtgatcggaaacaagcctgagagcgatatcctggtccataccgcctacgacga | |
| | gagtaccgacgaaaatgtgatgctgctgacatccgacgccccagagtataagccctgg | |
| | gctctggtcatccaggattccaacggagagaacaaaatcaaaatgctgtctggaggca | |
| | gcatgggaccattttgcctgggctgttctcaccggaagtgctatagccccatcagaaacc | |
| | tgatcagccaggagacattcaagtttcacttcaagaatctgggctacgccaagggccgg | |
| | aaggataccttcctgtgctatgaggtgacaagaaaggactgtgatagccccgtgtccct | |
| | gcaccacggcgtgtttaagaacaaggacaatatccacgccgagatctgttttctgtactg | |
| | gttccacgataaggtgctgaaggtgctgtcccccagggaggagttcaagatcacctggt | |
| | atatgtcttggagcccttgcttcgagtgtgccgagcagatcgtgcgctttctggccacac | |
| | accacaacctgagcctggacatctttagctcccggctgtacaacgtgcaggatcctgag | |
| | acacagcagaatctgtgcagactggtgcaggagggagcacaggtggcagcaatgga | |
| | cctgtatgagttcaagaagtgttggaagaagtttgtggataacggcggccggagattcc | |
| | ggccgtggaagagactgctgaccaacttccggtaccaggacagcaagctgcaggag | |
| | atcctgcgcccttgctatatctccgtgccatctagcgagaatctgtacttccagagcatgt | |
| | cctctagcaccctgtctaatatctgcctgaccaagggcctgcccgagacaaggttctgg | |
| | gtggagggccggagaatggaccctctgagcgaggaggagttttactcccagttctataa | |
| | ccagagggtgaagcacctgtgctactatcaccgcatgaagccttacctgtgctatcagct | |
| | ggagcagttcaatggacaggcaccactgaagggatgcctgctgtccgagaagggcaa | |
| | gcagcacgccgagatcctgtttctggataagatcagatctatggagctgagccaggtga | |
| | ccatcacatgttacctgacatggtccccatgcccaaactgtgcatggcagctggcagcc | |
| | ttcaagagggaccgcccagatctgatcctgcacatctacacctctaggctgtattttcact | |
| | ggaagcgccccttccagaagggcctgtgctccctgtggcagtctggcatcctggtgga | |
| | cgtgatggatctgccccagtttaccgactgttggacaaacttcgtgaatcctaagcggcc | |
| | attttggccctggaagggcctggagatcatcagcaggcgcacacagcggagactgag | |
| | gcgcatcaaggagtcctggggcctgcaggacctggtgaacgattttggcaatctgcag | |
| | ctgggaccccctatgagcaagcggcccgccgccaccaagaaggccggccaggcca | |
| | agaagaagaaggctactaacttcagcctgctgaagcaggctggggacgtggaggaga | |
| | accctggacctatgggcaacgccagaacaagacgggggagcgcagagccgagaa | |
| | gcaggcccaatggaaagccgctaattccggcgggagcagtggoggatccagtgggt | |
| | ccgaaacccctggcacaagcgaaagcgctacccctgagtccagcggagggtcctca | |
| | ggggaattcatgaaaagcatgtctagcatggtgtctgacacaagctgtaccttcccctcc | |
| | tctgatggcatcttttggaagcactggattcagacaaaggacggccagtgtggctcccct | |
| | ctggtgtctaccagggatggcttcatcgtgggcatccacagcgcctccaacttcacaaa | |
| | caccaacaactactttacatccgtgcctaagaacttcatggagctgctgaccaatcagga | |
| | ggcacagcagtgggtgtctggatggcgcctgaacgccgatagcgtgctgtggggcgg | |
| | ccacaaggtgtttatggtgaagccagaggagcccttccagcctgtgaaggaggccacc | |
| | cagaagcggcccgccgccaccaagaaggccggccaggccaagaagaagaaggag | |
| | ggcagaggaagtctgctaacatgcggtgacgtcgaggagaatcctggcccaatgggc | |
| | gagtccctgtttaagggcccacgggactacaaccccatcagctccacaatctgccacct | |
| | gaccaatgagagcgatggccacaccacatccctgtatggcatcggcttcggccccttca | |
| | tcatcacaaacaagcacctgtttcggagaaacaatggcaccctgctggtgcagtctctg | |
| | cacggcgtgttcaaggtgaagaataccacaaccctgcagcagcacctgatcgacgga | |
| | agggatatgatcatcatcagaatgcctaaggacttccccccttttccacagaagctgaag | |
| | tttcgggagccacagagggaggagaggatctgcctggtgacaaccaacttccagaca | |
| | ctcgagaagcggcccgccgccaccaagaaggccggccaggccaagaagaagaagt | |
| | aatgagctccgggtggcatccctgtgacccctccccagtgcctctcctggccctggaag | |
| | ttgccactccagtgcccaccagccttgtcctaataaaattaagttgcatcaagctgcggc | |
| | cgcaaaaaaaaaaaaaaaaaaaaaaaaaaaaaaaaaaaaaaaaaaaaaaaaaaaaa | |
| | aaaaaaaaaaaaaaaaaaaaaaaaaaaaaaaaaaaaaaaaaaaaaaa | |
| nCas9 mRNA | gggGGACAGATCGCCTGGAGACGCCATCCACGCTGTT | SEQ ID NO. |
| | TTGACCTCCATAGAAGACACCGGGACCGATCCAGCC | 361 |
| | TCCGCGGCCGGGAACGGTGCATTGGAACGCGGATTC | |
| | CCCGTGCCAAGAGTGACTCACCGTCCTTGACACGgcca | |
| | ccatgGCCCCTGCCGCCAAGCGGGTCAAGCTCGACGGT | |
| | ATCCACGGAGTCCCAGCAGCtatggacaagaagtacagcatcggcc | |
| | tggccatcggcaccaacagcgtgggctgggccgtgatcaccgacgagtacaaggtgc | |
| | ccagcaagaagttcaaggtgctgggcaacaccgaccggcacagcatcaagaagaac | |
| | ctgatcggcgccctgctgttcgacagcggcgagaccgccgaggccacccggctgaa | |
| | gcggaccgcccggcggcggtacacccggcggaagaaccggatctgctacctgcag | |
| | gagatcttcagcaacgagatggccaaggtggacgacagcttcttccaccggctggagg | |
| | agagcttcctggtggaggaggacaagaagcacgagcggcaccccatcttcggcaaca | |
| | tcgtggacgaggtggcctaccacgagaagtaccccaccatctaccacctgcggaaga | |
| | agctggtggacagcaccgacaaggccgacctgcggctgatctacctggccctggccc | |
| | acatgatcaagttccggggccacttcctgatcgagggcgacctgaaccccgacaacag | |
| | cgacgtggacaagctgttcatccagctggtgcagacctacaaccagctgttcgaggag | |
| | aaccccatcaacgccagcggcgtggacgccaaggccatcctgagcgcccggctgag | |
| | caagagccggcggctggagaacctgatcgcccagctgcccggcgagaagaagaac | |
| | ggcctgttcggcaacctgatcgccctgagcctgggcctgacccccaacttcaagagca | |
| | acttcgacctggccgaggacgccaagctgcagctgagcaaggacacctacgacgac | |
| | gacctggacaacctgctggcccagateggcgaccagtacgccgacctgttcctggcc | |
| | gccaagaacctgagcgacgccatcctgctgagcgacatcctgcgggtgaacaccgag | |
| | atcaccaaggcccccctgagcgccagcatgatcaagcggtacgacgagcaccacca | |
| | ggacctgaccctgctgaaggccctggtgcggcagcagctgcccgagaagtacaagg | |
| | agatcttcttcgaccagagcaagaacggctacgccggctacatcgacggcggcgcca | |
| | gccaggaggagttctacaagttcatcaagcccatcctggagaagatggacggcaccg | |
| | aggagctgctggtgaagctgaaccgggaggacctgctgcggaagcagcggaccttc | |
| | gacaacggcagcatcccccaccagatccacctgggcgagctgcacgccatcctgcgg | |
| | cggcaggaggacttctaccccttcctgaaggacaaccgggagaagatcgagaagatc | |
| | ctgaccttccggatcccctactacgtgggccccctggcccggggcaacagccggttcg | |
| | cctggatgacccgaaagagcgaggagaccatcaccccctggaacttogaggaggtg | |
| | gtggacaagggcgccagcgcccagagcttcatcgagoggatgaccaacttogacaag | |
| | aacctgcccaacgagaaggtgctgcccaagcacagcctgctgtacgagtacttcaccg | |
| | tgtacaacgagctgaccaaggtgaagtacgtgaccgagggcatgeggaagcccgcct | |
| | tcctgagcggcgagcagaagaaggccatcgtggacctgctgttcaagaccaaccgga | |
| | aggtgaccgtgaagcagctgaaggaggactacttcaagaagatcgagtgcttcgacag | |
| | cgtggagatcagcggcgtggaggaccggttcaacgccagcctgggcacctaccacg | |
| | acctgctgaagatcatcaaggacaaggacttcctggacaacgaggagaacgaggaca | |
| | tcctggaggacatcgtgctgaccctgaccctgttcgaggaccgggagatgatcgagga | |
| | gcggctaaagacctacgcccacctgttcgacgacaaggtgatgaagcagctgaagcg | |
| | gcggcggtacaccggctggggccggctgagccggaagctgatcaacggcatccgg | |
| | gacaagcagagcggcaagaccatcctggacttcctcaagagcgacggcttcgccaac | |
| | cggaacttcatgcagctgatccacgacgacagcctgaccttcaaggaggacatccaga | |
| | aggcccaggtgagcggccagggcgacagcctgcacgagcacatogccaacctggc | |
| | cggcagccccgccatcaagaagggcatcctgcagaccgtgaaggtggtggacgagc | |
| | tggtgaaggtgatgggccggcacaagcccgagaacatcgtgatcgagatggcccgg | |
| | gagaaccagaccacccagaagggccagaagaacagccgggagcggatgaagcgg | |
| | atcgaggagggcatcaaggagctgggcagccagatcctgaaggagcaccccgtgga | |
| | gaacacccagctgcagaacgagaagctgtacctgtactacctgcagaacggccggga | |
| | catgtacgtggaccaggagctggacatcaaccggctgagcgactacgacgtggacca | |
| | catcgtgccccagagcttcctgaaggacgacagcatcgacaacaaggtgctgacccg | |
| | gagcgacaagaaccggggcaagagcgacaacgtgcccagcgaggaggtggtgaa | |
| | gaagatgaagaactactggcggcagctgctgaacgccaagctgatcacccagoggaa | |
| | gttcgacaacctgaccaaggccgagcggggcggcctgagcgagctggacaaggcc | |
| | ggcttcatcaagcggcagctggtggagacccggcagatcaccaagcacgtggccca | |
| | gatcctggacagccggatgaacaccaagtacgacgagaacgacaagctgatccggg | |
| | aggtgaaggtgatcaccctcaagagcaagctggtgagcgacttccggaaggacttcca | |
| | gttctacaaggtgcgggagatcaacaactaccaccacgcccacgacgcctacctgaac | |
| | gccgtggtgggcaccgccctgatcaagaagtaccccaagctggagagcgagttcgtg | |
| | tacggcgactacaaggtgtacgacgtgcggaagatgatcgccaagagcgagcagga | |
| | gatcggcaaggccaccgccaagtacttcttctacagcaacatcatgaacttcttcaagac | |
| | cgagatcaccctggccaacggcgagatccggaagcggcccctgatcgagaccaacg | |
| | gcgagaccggcgagatcgtgtgggacaagggccgggacttcgccaccgtgcggaa | |
| | ggtgctgagcatgccccaggtgaacatcgtgaaaaagaccgaggtgcagaccggcg | |
| | gcttcagcaaggagagcatcctgcccaagcggaacagcgacaagctgatcgcccgg | |
| | aagaaggactgggaccccaagaagtacggcggcttcgacagccccaccgtggccta | |
| | cagcgtgctggtggtggccaaggtggagaagggcaagagcaagaagctcaagagc | |
| | gtgaaggagctgctgggcatcaccatcatggagcggagcagcttcgagaagaacccc | |
| | atcgacttcctggaggccaagggctacaaggaggtgaagaaggacctgatcatcaag | |
| | ctgcccaagtacagcctgttcgagctggagaacggccggaagcggatgctggccagc | |
| | gccggcgagctgcagaagggcaacgagctggccctgcccagcaagtacgtgaactt | |
| | cctgtacctggccagccactacgagaagctgaagggcagccccgaggacaacgagc | |
| | agaagcagctgttcgtggagcagcacaagcactacctggacgagatcatcgagcaga | |
| | tcagcgagttcagcaagcgggtgatcctggccgacgccaacctggacaaggtgctga | |
| | gcgcctacaacaagcaccgggacaagcccatccgggagcaggccgagaacatcatc | |
| | cacctgttcaccctgaccaacctgggcgcccccgccgccttcaagtacttogacaccac | |
| | catcgaccggaagcggtacaccagcaccaaggaggtgctggacgccaccctgatcc | |
| | accagagcatcaccggcctgtacgagacccggatcgacctgagccagctgggcggc | |
| | gacagcggcggcagcAAGCGGCCCGCCGCCACCAAGAAGG | |
| | CCGGCCAGGCCAAGAAGAAGAAGtaatgagctcCGGGTG | |
| | GCATCCCTGTGACCCCTCCCCAGTGCCTCTCCTGGCC | |
| | CTGGAAGTTGCCACTCCAGTGCCCACCAGCCTTGTCC | |
| | TAATAAAATTAAGTTGCATCAAGCTgoggccgcaaaaaaaaa | |
| | aaaaaaaaaaaaaaaaaaaaaaaaaaaaaaaaaaaaaaaaaaaaaaaaaaaaaaaa | |
| | aaaaaaaaaaaaaaaaaaaaaaaaaaaaaaaaaaa | |
| hg-PCSK9-Mouse- | ma*mc*mC*AGGGGAGGUUUGAGAGCUAGGCCAACA | SEQ ID NO. |
| end modified | UGAGGAUCACCCAUGUCUGCAGGGCCUAGCAAGUU | 362 |
| | CAAAUAAGGCUAGUCCGUUAUCAACUUGAAAAAGU | |
| | GGCACCGAGUCGG*mU*mG*mC | |
| mg-PCSK9- | mC*mA*mG*GUUCCAUGGGAUGCUCUGUUUGAGAGC | SEQ ID NO. |
| Mouse-end | UAGGGCCCUGAAGAAGGGCCCUAGCAAGUUCAAAU | 363 |
| modified | AAGGCUAGUCCGUUAUCAACUUGAAAAAGUGGCAC | |
| | CGAGUCGG*mU*mG*mC | |
| hcr-ANGPTL3- | mG*mA*mA*GAGGAAAGUUUGAGAGCUAUGCUGUU | SEQ ID NO. |
| Mouse | UUGGGCCAACAUGAGGAUCACCCAUGUCUGCAGGG | 364 |
| | CC*mU*mU*mU | |
| mcr-ANGPTL3- | mA*mC*mU*ACAAGUUAAAAACGAGGGUUUGAGAG | SEQ ID NO. |
| Mouse | CUAUGCUGUUUUGGGGCCCUGAAGAAGGGCCC*mU* | 365 |
| | mU*mU | |
| hcr-ANGPTL3- | mG*mA*mA*GAAGAAAGUUUGAGAGCUAUGCUGUU | SEQ ID NO. |
| Human | UUGGGCCAACAUGAGGAUCACCCAUGUCUGCAGGG | 366 |
| | CC*mU*mU*mU | |
| mcr-ANGPTL3- | mA*mC*mU*ACAAGUCAAAAAUGAAGGUUUGAGAG | SEQ ID NO. |
| Human | CUAUGCUGUUUUGGGGCCCUGAAGAAGGGCCC*mU* | 367 |
| | mU*mU | |
| hcr-ANGPTL3- | mG*mA*mA*GAAGAAAGUUUGAGAGCUAUGCUGUU | SEQ ID NO. |
| Monkey | UUGGGCCAACAUGAGGAUCACCCAUGUCUGCAGGG | 368 |
| | CC*mU*mU*mU | |
| mcr-ANGPTL3- | mA*mC*mU*ACAAGUCAAAAAUGAAGGUUUGAGAG | SEQ ID NO. |
| Monkey | CUAUGCUGUUUUGGGGCCCUGAAGAAGGGCCC*mU* | 369 |
| | mU*mU | |
| hcr-PCSK9-Mouse | UUCCUCUGUCGUUUGAGAGCUAUGCUGUUUUGGGC | SEQ ID NO. |
| | CAACAUGAGGAUCACCCAUGUCUGCAGGGCCUUU | 370 |
| mcr-PCSK9-Mouse | CAGGUUCCAUGGGAUGCUCUGUUUGAGAGCUAUGC | SEQ ID NO. |
| | UGUUUUGGGGCCCUGAAGAAGGGCCCUUU | 371 |
| hcr-PCSK9-Human | UUCAUCCGCCGUUUGAGAGCUAUGCUGUUUUGGGC | SEQ ID NO. |
| | CAACAUGAGGAUCACCCAUGUCUGCAGGGCCUUU | 372 |
| mcr-PCSK9- | CAGGUUCCACGGGAUGCUCUGUUUGAGAGCUAUGC | SEQ ID NO. |
| Human | UGUUUUGGGGCCCUGAAGAAGGGCCCUUU | 373 |
| hcr-PCSK9- | UUCAUCCGCCGUUUGAGAGCUAUGCUGUUUUGGGC | SEQ ID NO. |
| Monkey | CAACAUGAGGAUCACCCAUGUCUGCAGGGCCUUU | 374 |
| mcr-PCSK9- | CAGGUUCCAUGGGAUGCUCUGUUUGAGAGCUAUGC | SEQ ID NO. |
| Monkey | UGUUUUGGGGCCCUGAAGAAGGGCCCUUU | 375 |
| hcr-ANGPTL3- | GAAGAGGAAAGUUUGAGAGCUAUGCUGUUUUGGGC | SEQ ID NO. |
| Mouse | CAACAUGAGGAUCACCCAUGUCUGCAGGGCCUUU | 376 |
| mcr-ANGPTL3- | ACUACAAGUUAAAAACGAGGGUUUGAGAGCUAUGC | SEQ ID NO. |
| Mouse | UGUUUUGGGGCCCUGAAGAAGGGCCCUUU | 377 |
| hcr-ANGPTL3- | GAAGAAGAAAGUUUGAGAGCUAUGCUGUUUUGGGC | SEQ ID NO. |
| Human | CAACAUGAGGAUCACCCAUGUCUGCAGGGCCUUU | 378 |
| mcr-ANGPTL3- | ACUACAAGUCAAAAAUGAAGGUUUGAGAGCUAUGC | SEQ ID NO. |
| Human | UGUUUUGGGGCCCUGAAGAAGGGCCCUUU | 379 |
| hcr-ANGPTL3- | GAAGAAGAAAGUUUGAGAGCUAUGCUGUUUUGGGC | SEQ ID NO. |
| Monkey | CAACAUGAGGAUCACCCAUGUCUGCAGGGCCUUU | 380 |
| mcr-ANGPTL3- | ACUACAAGUCAAAAAUGAAGGUUUGAGAGCUAUGC | SEQ ID NO. |
| Monkey | UGUUUUGGGGCCCUGAAGAAGGGCCCUUU | 381 |
| HBG-hcrRNA-1 | ACUCCACCCAGUUUGAGAGCUAGGCCAACAUGAGG | SEQ ID NO. |
| | AUCACCCAUGUCUGCAGGGCCUGCUGUUUUG | 382 |
| HBG-hcrRNA-2 | ACUCCACCCAGUUUGAGAGCUAUGCUGGGCCAACA | SEQ ID NO. |
| | UGAGGAUCACCCAUGUCUGCAGGGCCUUUUG | 383 |
| HBG-hcrRNA-3 | ACUCCACCCAGUUUGAGAGCUAUGCUGUUUUGGGC | SEQ ID NO. |
| | CAACAUGAGGAUCACCCAUGUCUGCAGGGCC | 384 |
| HBG-mcrRNA-1 | CUUGACCAAUAGCCUUGACAGUUUGAGAGCUAGGG | SEQ ID NO. |
| | CCCUGAAGAAGGGCCCUGCUGUUUUG | 385 |
| HBG-mcrRNA-2 | CUUGACCAAUAGCCUUGACAGUUUGAGAGCUAUGC | SEQ ID NO. |
| | UGGGGCCCUGAAGAAGGGCCCUUUUG | 386 |
| HBG-mcrRNA-3 | CUUGACCAAUAGCCUUGACAGUUUGAGAGCUAUGC | SEQ ID NO. |
| | UGUUUUGGGGCCCUGAAGAAGGGCCC | 387 |
| HBG-mcrRNA-1- | CUUGACCAAUAGCCUUGACAGUUUGAGAGCUAGGG | SEQ ID NO. |
| 2xboxB | CCCUGAAGAAGGGCGGGCCCUGAAGAAGGGCCCAA | 388 |
| | CCUGCUGUUUUG | |
| HBG-mcrRNA-O | CUUGACCAAUAGCCUUGACAGUUUGAGAGCUAUGC | SEQ ID NO. |
| | UGUUUUG | 389 |
| HBG-hcrRNA-O | ACUCCACCCAGUUUGAGAGCUAUGCUGUUUUG | SEQ ID NO. |
| | | 390 |
| HBG-hcrRNA-3U | mA*mC*mU*CCACCCAGUUUGAGAGCUAUGCUGUUU | SEQ ID NO. |
| | UGGGCCAACAUGAGGAUCACCCAUGUCUGCAGGGC | 391 |
| | C*mU*mU*mU | |
| HBG-hcrRNA-4U | mA*mC*mU*CCACCCAGUUUGAGAGCUAUGCUGUUU | SEQ ID NO. |
| | UGGGCCAGCGAGGAGCACCAGCCCUCGCGUGUGAC | 392 |
| | GAUGACGAUCACGCAUCG*mU*mU*mU | |
| HBG-hcrRNA-5U | mA*mC*mU*CCACCCAGUUUGAGAGCUAUGCUGUUU | SEQ ID NO. |
| | UGGGCCAGGAAUGACCACCAGGCAUUCCGAUCCGA | 393 |
| | CGAUGGACCAUCAGGCCAUCG*mU*mU*mU | |
| HBG-mcrRNA-3U | mC*mU*mU*GACCAAUAGCCUUGACAGUUUGAGAGC | SEQ ID NO. |
| | UAUGCUGUUUUGGGGCCCUGAAGAAGGGCCC*mU* | 394 |
| | mU*mU | |
| HBG-mcrRNA-4U | mC*mU*mU*GACCAAUAGCCUUGACAGUUUGAGAGC | SEQ ID NO. |
| | UAUGCUGUUUUGGGGCCCUGAAGAAGGGCCCGGGC | 395 |
| | CCUGAAGAAGGGCCC*mU*mU*mU | |

### Examples

### RNA Preparation

mcrRNAs targeting the HBG gene, the PCSK9 gene, and the ANGPTL3 gene, hcrRNAs targeting the upstream region of the mcrRNA targeting site, and tracrRNAs were designed. The sequence of the mcrRNA, hcrRNA, and tracrRNA are shown in Table 9. The combinations of mcrRNA, hcrRNA, and tracrRNA used in this example are shown in Table 10.

Chemically end-modified hcrRNA, mcrRNA and tracrRNA (2′-O-methyl and 3′-phosphorothioate linkage modifications were made to the first and last three nucleotides) were synthesized by GenScript. mRNAs encoding the V5 LigoRNA-tCBE system (as exemplified in FIG. 1A) were transcribed in vitro.

### Cell Culture and Transfection

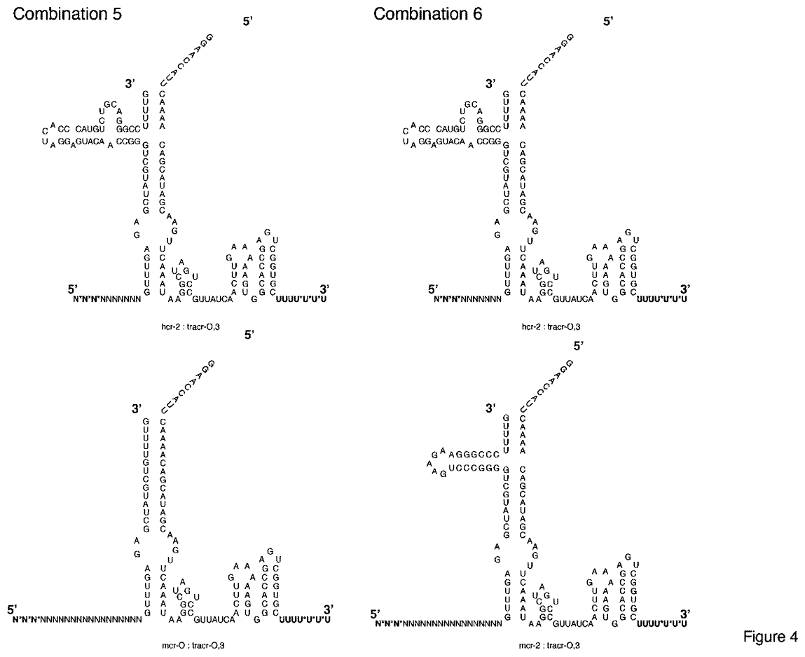

Human CD34⁺ hematopoietic stem and progenitor cells (HSPCs) were electroporated with the end-modified hcrRNA, mcrRNA, and tracrRNA, as well as the mRNAs described above. Electroporation was performed using Lonza 4D Nucleofector by using officially recommended program (e.g., EO-100). For 20-μl Nucleocuvette Strips, 0.2 million HSPCs were resuspended in 20 μl P3 Primary Cell 4D-Nucleofector buffer and about 400 pmol RNA complex were added. The editing frequencies of HBG target sequence were measured with cells cultured in medium 48 hours after electroporation. It was found that multiple hcr-tracrRNA and mcr-tracrRNA combinations induced efficient base editing, particularly hcr-tracrRNA-3 and mcr-tracrRNA-3 combination (FIG. 4).

HepG2, Hepa1-6 or COS-1 cells were electroporated with the end-modified hcrRNA, mcrRNA, tracrRNA and the mRNAs described above. Electroporation was performed using Lonza 4D Nucleofector by using officially recommended program (e.g., EH-100). For 20-μl Nucleocuvette Strips, 0.2 million cells were resuspended in 20 μl SF Cell Line 4D-Nucleofector buffer and about 400 pmol RNA complex or indicated RNA dosage were added.

Base substitution frequency at each target sites was calculated by EditR analysis. See http://baseeditr.com/.

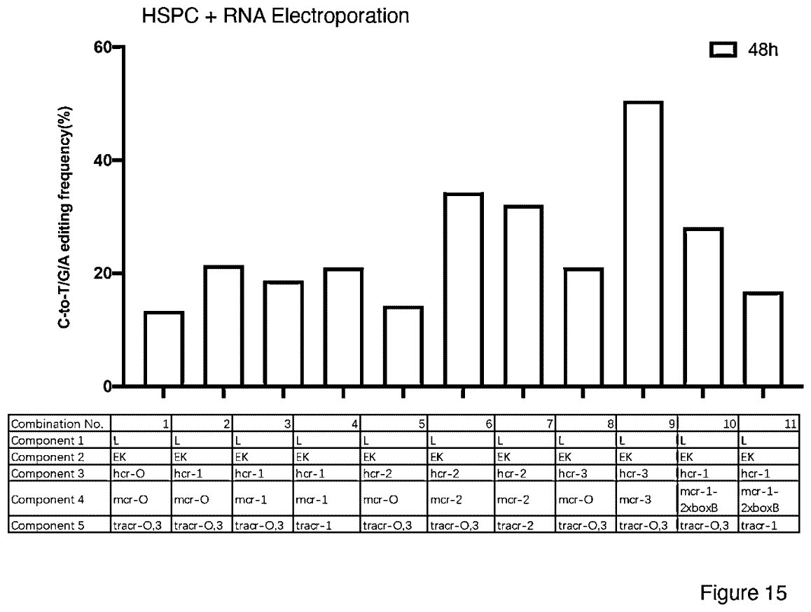

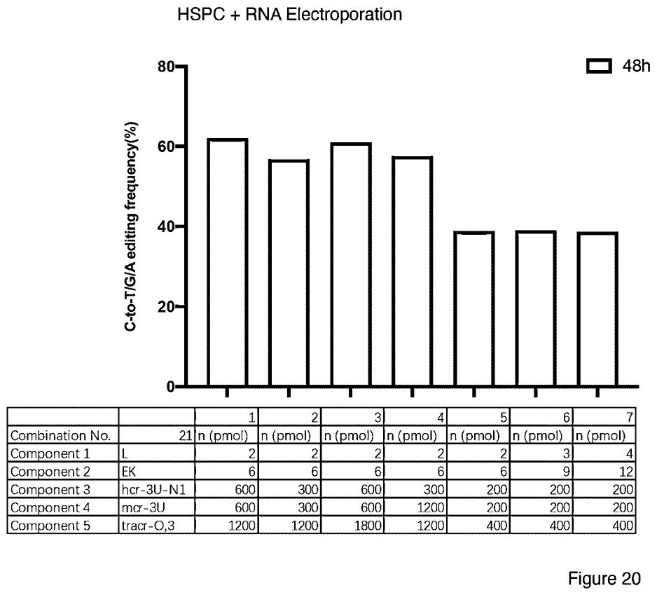

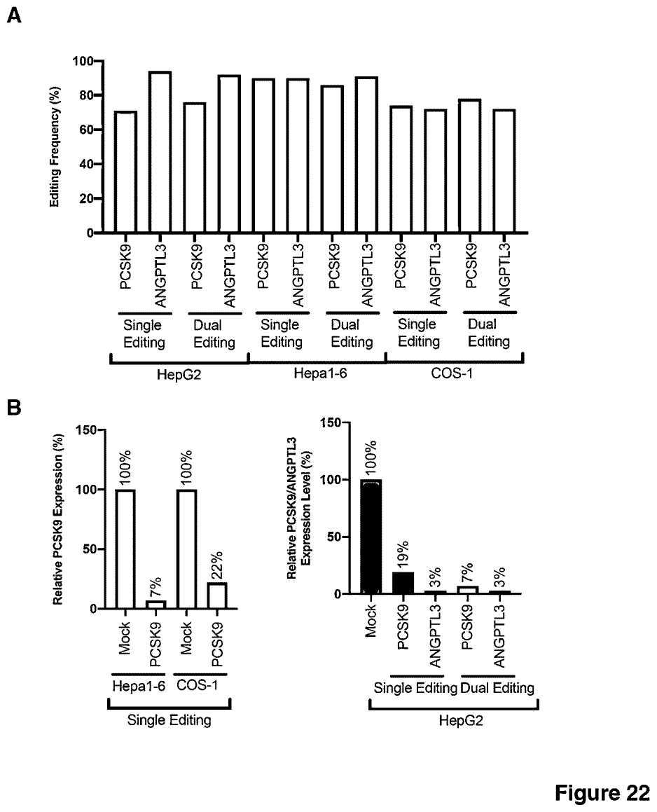

The results of base editing frequencies are shown in FIGS. 15-22.

### ELISA Analysis

The protein levels were determined using commercially available ELISA kit according to the manufacturer's protocol. The luminescence signal of the ELISA assays was collected by spectraMax M5e microplate reader.

### Animal Studies

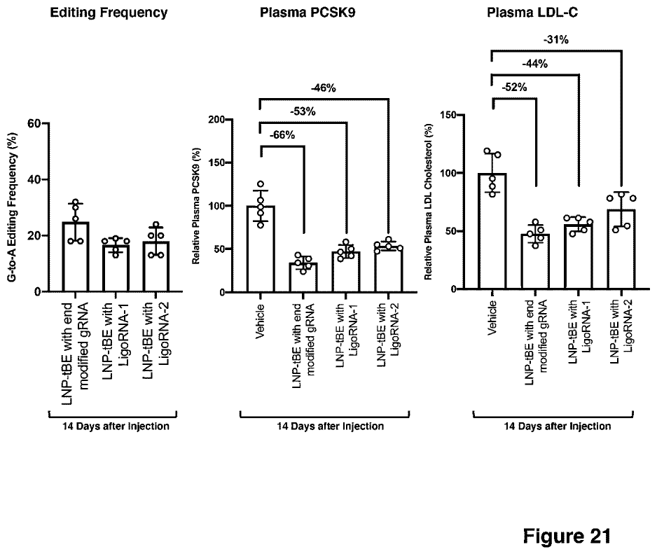

Mice were given free access to food and water, and were maintained under a 12 h-12 h light-dark cycle with controlled temperature (20-25° C.) and humidity (50±10%). LNP vector comprising the tBE editors and end-modified hcrRNA, mcrRNA, tracrRNA and the mRNAs described above targeting PCSK9 gene (SEQ ID NOs: 22, 302-303 and 360-363) was delivered to at least four female C57BL/6 mice (aged 6-8 weeks) intravenously through tail vein injection. Mice were fasted for 5 h before blood was collected before liver perfusion. Two or four weeks after injection, the blood was collected, and the plasma was separated by centrifugation. Plasma levels of PCSK9 and LDL-C were measured using the Mouse PCSK9 ELISA Kit, triglyceride kit, and LDL-C kit, respectively. The genomiic DNA from mouse tissues were isolated using the E.Z.N.A. Tissue DNA Kit. The results of editing frequency and plasma levels of PCSK9 and LDL-C are shown in FIG. 21.

TABLE 9
The sequence and modification information of the original
types and hairpin fused types of crRNA and tracrRNA.

| SEQ ID | | | base |
|---|---|---|---|
| NO: | Name | RNA sequence | number |
| 21 | HBG-hcrRNA-O | mA*mC*mU*CCACCCAGUUUGAGAGCUAUGC | 32 |
| | | UGUU*mU*mU*mG | |
| 13 | HBG-hcrRNA-1 | mA*mC*mU*CCACCCAGUUUGAGAGCUAGGC | 66 |
| | | CAACAUGAGGAUCACCCAUGUCUGCAGGGC | |
| | | CUGCUGUU*mU*mU*mG | |
| 14 | HBG-hcrRNA-2 | mA*mC*mU*CCACCCAGUUUGAGAGCUAUGC | 66 |
| | | UGGGCCAACAUGAGGAUCACCCAUGUCUGC | |
| | | AGGGCCUU*mU*mU*mG | |
| 15 | HBG-hcrRNA-3 | mA*mC*mU*CCACCCAGUUUGAGAGCUAUGC | 66 |
| | | UGUUUUGGGCCAACAUGAGGAUCACCCAUG | |
| | | UCUGCAGG*mG*mC*mC | |
| 20 | HBG-mcrRNA-O | mC*mU*mU*GACCAAUAGCCUUGACAGUUUG | 42 |
| | | AGAGCUAUGCUGUU*mU*mU*mG | |
| 19 | HBG-mcrRNA- | mC*mU*mU*GACCAAUAGCCUUGACAGUUUG | 82 |
| | 1-2xboxB | AGAGCUAGGGCCCUGAAGAAGGGGGGCCC | |
| | | UGAAGAAGGGCCCAACCUGCUGUU*mU*mU* | |
| | | mG | |
| 16 | HBG-mcrRNA-1 | mC*mU*mU*GACCAAUAGCCUUGACAGUUUG | 61 |
| | | AGAGCUAGGGCCCUGAAGAAGGGCCCUGCU | |
| | | GUU*mU*mU*mG | |
| 17 | HBG-mcrRNA-2 | mC*mU*mU*GACCAAUAGCCUUGACAGUUUG | 61 |
| | | AGAGCUAUGCUGGGGCCCUGAAGAAGGGCC | |
| | | CUU*mU*mU*mG | |
| 18 | HBG-mcrRNA-3 | mC*mU*mU*GACCAAUAGCCUUGACAGUUUG | 61 |
| | | AGAGCUAUGCUGUUUUGGGGCCCUGAAGAA | |
| | | GGG*mC*mC*mC | |
| 22 | tracrRNA-O, 3 | mG*mG*mA*ACCAUUCAAAACAGCAUAGCAA | 86 |
| | | GUUCAAAUAAGGCUAGUCCGUUAUCAACUU | |
| | | GAAAAAGUGGCACCGAGUCGGUGCUUUU*m | |
| | | U*mU*mU | |
| 23 | tracrRNA-1 | mG*mG*mA*ACCAUUCAAAACAGCAAAAUA | 89 |
| | | GCAAGUUCAAAUAAGGCUAGUCCGUUAUCA | |
| | | ACUUGAAAAAGUGGCACCGAGUCGGUGCUU | |
| | | UU*mU*mU*mU | |
| 24 | tracrRNA-2 | mG*mG*mA*ACCAUUCAAAAAAACAGCAUA | 89 |
| | | GCAAGUUCAAAUAAGGCUAGUCCGUUAUCA | |
| | | ACUUGAAAAAGUGGCACCGAGUCGGUGCUU | |
| | | UU*mU*mU*mU | |
| 154 | HBG-hcrRNA- | mA*mC*mU*CCACCCAGUUUGAGAGCUAUGC | 86 |
| | 3U | UGUUUUGGGCCAACAUGAGGAUCACCCAUG | |
| | | UCUGCAGGGCC*mU*mU*mU | |
| 155 | HBG-hcrRNA- | mA*mC*mU*CCACCCAGUUUGAGAGCUAUGC | 89 |
| | 4U | UGUUUUGGGCCAGCGAGGAGCACCAGCCCU | |
| | | CGCGUGUGACGAUGACGAUCACGCAUCG*m | |
| | | U*mU*mU | |
| 156 | HBG-hcrRNA- | mA*mC*mU*CCACCCAGUUUGAGAGCUAUGC | 83 |
| | 5U | UGUUUUGGGCCAGGAAUGACCACCAGGCAU | |
| | | UCCGAUCCGACGAUGGACCAUCAGGCCAUC | |
| | | G*mU*mU*mU | |
| 157 | HBG-mcrRNA- | mC*mU*mU*GACCAAUAGCCUUGACAGUUUG | 64 |
| | 3U | AGAGCUAUGCUGUUUUGGGGCCCUGAAGAA | |
| | | GGGCCC*mU*mU*mU | |
| 158 | HBG-mcrRNA- | mC*mU*mU*GACCAAUAGCCUUGACAGUUUG | 69 |
| | 4U | AGAGCUAUGCUGUUUUGGGGCCCUGAAGAA | |
| | | GGGCCCGGGCCCUGAAGAAGGGCCC*mU*mU | |
| | | *mU | |
| 302 | hcr-PCSK9- | mU*mU*mC*CUCUGUCGUUUGAGAGCUAUGC | 69 |
| | Mouse | UGUUUUGGGCCAACAUGAGGAUCACCCAUG | |
| | | UCUGCAGGGCC*mU*mU*mU | |
| 303 | mcr-PCSK9- | mC*mA*mG*GUUCCAUGGGAUGCUCUGUUU | 64 |
| | Mouse | GAGAGCUAUGCUGUUUUGGGGCCCUGAAGA | |
| | | AGGGCCC*mU*mU*mU | |
| 304 | hcr-PCSK9- | mU*mU*mC*AUCCGCCGUUUGAGAGCUAUGC | 69 |
| | Human | UGUUUUGGGCCAACAUGAGGAUCACCCAUG | |
| | | UCUGCAGGGCC*mU*mU*mU | |
| 305 | mcr-PCSK9- | mC*mA*mG*GUUCCACGGGAUGCUCUGUUUG | 64 |
| | Human | AGAGCUAUGCUGUUUUGGGGCCCUGAAGAA | |
| | | GGGCCC*mU*mU*mU | |
| 306 | hcr-PCSK9- | mU*mU*mC*AUCCGCCGUUUGAGAGCUAUGC | 69 |
| | Monkey | UGUUUUGGGCCAACAUGAGGAUCACCCAUG | |
| | | UCUGCAGGGCC*mU*mU*mU | |
| 307 | mcr-PCSK9- | mC*mA*mG*GUUCCAUGGGAUGCUCUGUUU | 64 |
| | Monkey | GAGAGCUAUGCUGUUUUGGGGCCCUGAAGA | |
| | | AGGGCCC*mU*mU*mU | |
| 308 | hcr-ANGPTL3- | mG*mA*mA*GAGGAAAGUUUGAGAGCUAUG | 69 |
| | Mouse | CUGUUUUGGGCCAACAUGAGGAUCACCCAU | |
| | | GUCUGCAGGGCC*mU*mU*mU | |
| 309 | mcr-ANGPTL3- | mA*mC*mU*ACAAGUUAAAAACGAGGGUUU | 64 |
| | Mouse | GAGAGCUAUGCUGUUUUGGGGCCCUGAAGA | |
| | | AGGGCCC*mU*mU*mU | |
| 310 | hcr-ANGPTL3- | mG*mA*mA*GAAGAAAGUUUGAGAGCUAUG | 69 |
| | Human | CUGUUUUGGGCCAACAUGAGGAUCACCCAU | |
| | | GUCUGCAGGGCC*mU*mU*mU | |
| 311 | mcr-ANGPTL3- | mA*mC*mU*ACAAGUCAAAAAUGAAGGUUU | 64 |
| | Human | GAGAGCUAUGCUGUUUUGGGGCCCUGAAGA | |
| | | AGGGCCC*mU*mU*mU | |
| 312 | hcr-ANGPTL3- | mG*mA*mA*GAAGAAAGUUUGAGAGCUAUG | 69 |
| | Monkey | CUGUUUUGGGCCAACAUGAGGAUCACCCAU | |
| | | GUCUGCAGGGCC*mU*mU*mU | |
| 313 | mcr-ANGPTL3- | mA*mC*mU*ACAAGUCAAAAAUGAAGGUUU | 64 |
| | Monkey | GAGAGCUAUGCUGUUUUGGGGCCCUGAAGA | |
| | | AGGGCCC*mU*mU*mU | |
| | | m: 2′-O-methyl | |
| | | *: 3′ phosphonothioate linkage | |

TABLE 10
26 different combinations of hcr-tracrRNA and
mcr-tracrRNA structure as used in FIGS. 15-22.

| Combination | hcrRNA | mcrRNA | tracrRNA |
|---|---|---|---|
| 1 | HBG-hcrRNA-O | HBG-mcrRNA-O | tracrRNA-O,3 |
| 2 | HBG-hcrRNA-1 | HBG-mcrRNA-O | tracrRNA-O,3 |
| 3 | HBG-hcrRNA-1 | HBG-mcrRNA-1 | tracrRNA-O,3 |
| 4 | HBG-hcrRNA-1 | HBG-mcrRNA-1 | tracrRNA-1 |
| 5 | HBG-hcrRNA-2 | HBG-mcrRNA-O | tracrRNA-O,3 |
| 6 | HBG-hcrRNA-2 | HBG-mcrRNA-2 | tracrRNA-O,3 |
| 7 | HBG-hcrRNA-2 | HBG-mcrRNA-2 | tracrRNA-2 |
| 8 | HBG-hcrRNA-3 | HBG-mcrRNA-O | tracrRNA-O,3 |
| 9 | HBG-hcrRNA-3 | HBG-mcrRNA-3 | tracrRNA-O,3 |
| 10 | HBG-hcrRNA-1 | HBG-mcrRNA-1-2xboxB | tracrRNA-O,3 |
| 11 | HBG-hcrRNA-1 | HBG-mcrRNA-1-2xboxB | tracrRNA-1 |
| 12 | HBG-hcrRNA-1 | HBG-mcrRNA-2 | tracrRNA-O,3 |
| 13 | HBG-hcrRNA-3 | HBG-mcrRNA-2 | tracrRNA-O,3 |
| 14 | HBG-hcrRNA-1 | HBG-mcrRNA-2 | tracrRNA-2 |
| 15 | HBG-hcrRNA-3 | HBG-mcrRNA-2 | tracrRNA-2 |
| 16 | HBG-hcrRNA-1 | HBG-mcrRNA-3 | tracrRNA-O,3 |
| 17 | HBG-hcrRNA-2 | HBG-mcrRNA-3 | tracrRNA-O,3 |
| 18 | HBG-hcrRNA-2 | HBG-mcrRNA-3 | tracrRNA-1 |
| 19 | HBG-hcrRNA-3 | HBG-mcrRNA-3 | tracrRNA-1 |
| 20 | HBG-hcrRNA-3 | HBG-mcrRNA-1 | tracrRNA-1 |
| 21 | HBG-hcrRNA-3U | HBG-mcrRNA-3U | tracrRNA-O,3 |
| 22 | HBG-hcrRNA-4U | HBG-mcrRNA-3U | tracrRNA-O,3 |
| 23 | HBG-hcrRNA-5U | HBG-mcrRNA-3U | tracrRNA-O,3 |
| 24 | HBG-hcrRNA-3U | HBG-mcrRNA-4U | tracrRNA-O,3 |
| 25 | HBG-hcrRNA-4U | HBG-mcrRNA-4U | tracrRNA-O,3 |
| 26 | HBG-hcrRNA-5U | HBG-mcrRNA-4U | tracrRNA-O,3 |
| 27 | hcr-PCSK9-Mouse | mcr-PCSK9-Mouse | tracrRNA-O,3 |
| 28 | hcr-PCSK9-Human | mcr-PCSK9-Human | tracrRNA-O,3 |
| 29 | hcr-PCSK9-Monkey | mcr-PCSK9-Monkey | tracrRNA-O,3 |
| 30 | hcr-ANGPTL3-Mouse | mcr-ANGPTL3-Mouse | tracrRNA-O,3 |
| 31 | hcr-ANGPTL3-Human | mcr-ANGPTL3-Human | tracrRNA-O,3 |
| 32 | hcr-ANGPTL3-Monkey | mcr-ANGPTL3-Human | tracrRNA-O,3 |

TABLE 12
amino acid sequence SEQ ID NO: 159-251

| | | SEQ ID |
|---|---|---|
| Name | Sequence | NO |
| Agrobacterium | MAERTHFMELALVEARSAGERDEVPIGAVLVLDGRVIARS | SEQ ID |
| fabrum TadA | GNRTRELNDVTAHAEIAVIRMACEALGQERLPGADLYVTL | NO. 159 |
| | EPCTMCAAAISFARIRRLYYGAQDPKGGAVESGVRFFSQP | |
| | TCHHAPDVYSGLAESESAEILRQFFREKRLDD | |
| Arabidopsis | MFNTYTNSLQWPIRSRNQQDYCSLLPERSESYKLSKAYTSS | SEQ ID |
| thaliana TadA | RCYCVSSRSSCCCCCSTPSSSSFVKPKVLINPGFVLYGVRQS | NO. 160 |
| | TLIQWPSFQRRLLVGGGRLMGCEVYSSCDGIRRKNRSFKL | |
| | RCLEESDECCGGRSCSDDVEAMISFLSEELIDEERKWNLVS | |
| | RVKEKKKVGNVRKVSVEGSNSYGNGRVSQRVKKPEGFGR | |
| | RKEIKEDVKLNERYDCEHCGRRKKSSELESESRRGSKLVT | |
| | GEYIGKSYRGDEEREVRPRRRKSSSCSSYYSLASSGEFESD | |
| | TEDQEEDVEIYRENVRSSEKKVVDQSAKRLKSRKEASQM | |
| | HSRKKRDESSTGVDSRYQKQIFEEGENSNQAVTLNQRRRK | |
| | KFSQTENRVSESTGNYEEDMEIHEVHVNDAETSSQNQKLF | |
| | NEREDYRVHSIRNDSGNENIESSQHQLKERLETRYSSEDRV | |
| | SEMRRRTKYSSSQEEGINVLQNFPEVTNNQQPLVEERISKQ | |
| | AGTRRTTEHISESSEIHDIDIRNTYVSQREDQIRNQEVHAGL | |
| | VSGLQSERKQQDYHIEHNPLQTTQSDRTSVSVSHTSDAVR | |
| | YTEIQRKSEKRLIGQGSTTAVQSDSKVEKNGAQKEDSRLD | |
| | HANSKKDGQTTLGLQSYQSKLSEEASSSQSSLMASRTKLQ | |
| | LVDLVSEEMQGSETTLIPPSSQLVSRRSGQSYRTGGVSIQEI | |
| | SHGTSESGYTTAFEHPRAGASVNSQSAGELMGFTSHEDAM | |
| | GSAHRLEQASEKYVGEFVKKAKHGVINPETEEQRAESNQL | |
| | KRRDSRRSSGGSGAKGPSDEMWVTDSAQGTPHPGATEGN | |
| | AAVGNAIFKRNGRSLWNVIADIARLRWGSRAGSPDSSAKP | |
| | AGRSSPNESVSSATWFSGREHDGSSDDNTKGDKVLPQEAP | |
| | SLHQVEVGQTSPRSQSEYPGTTKLKQRSERHEGVVSSPSST | |
| | ILEGGSVSNRMSSTSGNQIVGVDEEEGGNFEFRLPETALTE | |
| | VPMKLPSRNLIRSPPIKESSESSLTEASSDQNFTVGEGRRYP | |
| | RMDAGQNPLLFPGRNLRSPAVMEPPVPRPRMVSGSSSLRE | |
| | QVEQQQPLSAKSQEETGSVSADSALIQRKLQRNKQVVRDS | |
| | FEEWEEAYKVEAERRTVDEIFMREALVEAKKAADTWEVP | |
| | VGAVLVHDGKIIARGYNLVEELRDSTAHAEMICIREGSKA | |
| | LRSWRLADTTLYVTLEPCPMCAGAILQARVNTLVWGAPN | |
| | KLLGADGSWIRLFPGGEGNGSEASEKPPPPVHPFHPKMTIR | |
| | RGVLESECAQTMQQFFQLRRKKKDKNSDPPTPTDHHHHH | |
| | LPKLLNKMHQVLPFFCL | |
| Aquifex aeolicus | MGKEYFLKVALREAKRAFEKGEVPVGAIIVKEGEIISKAHN | SEQ ID |
| TadA | SVEELKDPTAHAEMLAIKEACRRLNTKYLEGCELYVTLEP | NO. 161 |
| | CIMCSYALVLSRIEKVIFSALDKKHGGVVSVFNILDEPTLN | |
| | HRVKWEYYPLEEASELLSEFFKKLRNNII | |
| Streptococcus | MPYSLEEQTYFMQEALKEAEKSLQKAEIPIGCVIVKDGEII | SEQ ID |
| pyogenes serotype | GRGHNAREESNQAIMHAEMMAINEANAHEGNWRLLDTT | NO. 162 |
| M3 TadA | LFVTIEPCVMCSGAIGLARIPHVIYGASNQKFGGADSLYQIL | |
| | TDERLNHRVQVERGLLAADCANIMQTFFRQGRERKKIAK | |
| | HLIKEQSDPFD | |
| Bacillus subtilis | MTQDELYMKEAIKEAKKAEEKGEVPIGAVLVINGEIIARA | SEQ ID |
| TadA | HNLRETEQRSIAHAEMLVIDEACKALGTWRLEGATLYVTL | NO. 163 |
| | EPCPMCAGAVVLSRVEKVVFGAFDPKGGCSGTLMNLLQE | |
| | ERFNHQAEVVSGVLEEECGGMLSAFFRELRKKKKAARKN | |
| | LSE | |
| Haemophilus | MDAAKVRSEFDEKMMRYALELADKAEALGEIPVGAVLV | SEQ ID |
| influenzae TadA | DDARNIIGEGWNLSIVQSDPTAHAEIIALRNGAKNIQNYRL | NO. 164 |
| | LNSTLYVTLEPCTMCAGAILHSRIKRLVFGASDYKTGAIGS | |
| | RFHFFDDYKMNHTLEVTSGVLAEECSQKLSTFFQKRREEK | |
| | KIEKALLKSLSDK | |
| Buchnera aphidicola | MKYEKDKNWMKIALKYAYYAKEKGEIPIGAILVFKERIIGI | SEQ ID |
| TadA | GWNSSISKNDPTAHAEIIALRGAGKKIKNYRLLNTTLYVTL | NO. 165 |
| | QPCIMCCGAIIQSRIKRLVFGANCNSSDHRFSLKNLFCDPQ | |
| | KDYKLDIKKNVMQRECSDILINFFQKKRKNKIHICKKI | |
| Escherichia coli | MSEVEFSHEYWMRHALTLAKRAWDEREVPVGAVLVHNN | SEQ ID |
| TadA | RVIGEGWNRPIGRHDPTAHAEIMALRQGGLVMQNYRLID | NO. 166 |
| | ATLYVTLEPCVMCAGAMIHSRIGRVVFGARDAKTGAAGS | |
| | LMDVLHHPGMNHRVEITEGILADECAALLSDFFRMRRQEI | |
| | KAQKKAQSSTD | |
| Streptococcus | MPYSLEEQTYFMQEALKEAEKSLQKAEIPIGCVIVKDGEII | SEQ ID |
| pyogenes serotype | GRGHNAREESNQAIMHAEMMAINEANAHEGNWRLLDTT | NO. 167 |
| M1 TadA | LFVTIEPCVMCSGAIGLARIPHVIYGASNQKFGGVDSLYQIL | |
| | TDERLNHRVQVERGLLAADCANIMQTFFRQGRERKKIAK | |
| | HLIKEQSDPFD | |
| Rickettsia bellii | MREALKQAEIAFSKNEVPVGAVIVDRENQKIISKSYNNTEE | SEQ ID |
| TadA | KNNALYHAEIIAINEACRIISSKNLSDYDIYVTLEPCAMCAA | NO. 168 |
| | AIAHSRLKRLFYGASDSKHGAVESNLRYFNSKACFHRPEIY | |
| | SGIFAEDSALLMKGFFKKIRD | |
| Rickettsia felis | MEQALKQAGIAFDKNEVPVGAVIVDRLNQKIIVSSHNNTE | SEQ ID |
| TadA | EKNNALYHAEIIAINEACNLISSKNLNDYDIYVTLEPCAMC | NO. 169 |
| | AAAIAHSRLKRLFYGASDSKHGAVESNLRYFNSSVCFYRP | |
| | EIYSGILAEDSRLLMKEFFKRIR | |
| Salmonella | MSDVELDHEYWMRHALTLAKRAWDEREVPVGAVLVHN | SEQ ID |
| typhimurium TadA | HRVIGEGWNRPIGRHDPTAHAEIMALRQGGLVLQNYRLL | NO. 170 |
| | DTTLYVTLEPCVMCAGAMVHSRIGRVVFGARDAKTGAAG | |
| | SLIDVLHHPGMNHRVEIIEGVLRDECATLLSDFFRMRRQEI | |
| | KALKKADRAEGAGPAV | |
| Escherichia coli O6 | MSEVEFSHEYWMRHAMTLAKRAWDEREVPVGAVLVHN | SEQ ID |
| TadA | NRVIGEGWNRPIGRHDPTAHAEIMALRQGGLVMQNYRLI | NO. 171 |
| | DATLYVTLEPCVMCAGAMIHSRIGRVVFGARDAKTGAAG | |
| | SLMDVLHHPGMNHRVEITEGILADECAALLSDFFRMRRQE | |
| | IKAQKKAQSSTD | |
| Buchnera aphidicola | MKSNRDSYWMKIALKYAYYAEENGEVPIGAILVFQEKIIG | SEQ ID |
| subsp. Baizongia | TGWNSVISQNDSTAHAEIIALREAGRNIKNYRLVNTTLYVT | NO. 172 |
| pistaciae (strain Bp) | LQPCMMCCGAIINSRIKRLVFGASYKDLKKNPFLKKIFINL | |
| TadA | EKNKLKIKKHIMRNECAKILSNFFKNKRF | |
| Streptococcus | MPYSLEEQTYFMQEALKEAEKSLQKAEIPIGCVIVKDGEII | SEQ ID |
| pyogenes serotype | GRGHNAREESNQAIMHAEMMAINEANAHEGNWRLLDTT | NO. 173 |
| M18 TadA | LFVTIEPCVMCSGAIGLARIPHVIYGASNQKFGGADSLYQIL | |
| | TDERLNHRVQVERGLLAADCANIMQTFFRQGRERKKNS | |
| Rickettsia prowazekii | MEQALKQARLAFDKNEVPVGVVIVCRLNQKIIVSSHNNIE | SEQ ID |
| TadA | EKKNPLCHAEIIAINTACNLISSKNLNDYDIYVTLEPCAMC | NO. 174 |
| | ASAISHSRLKRLFYGASDSKHGAVESNLRYFNSNSCFYRPE | |
| | IYSGILSEHSRFLMQEFFQRIRSAID | |
| Rickettsia typhi | MEQALKQARLAFDKNEVPVGVVIVYRLNQKIIVSSHNNIE | SEQ ID |
| TadA | EKNNALCHAEIIAINEACNLISSKNLNDYDIYVTLEPCAMC | NO. 175 |
| | ASAISHSRLKRLFYGASDSKQGAVESNLRYFNSSACFHRPE | |
| | IYSGILSEHSRFLMKEFFQKMRSTID | |
| Buchnera aphidicola | MHDSDKYFMKCAIFLAKISEMIGEVPVGAVLVFNNTIIGKG | SEQ ID |
| subsp. Schizaphis | LNSSILNHDPTAHAEIKALRNGAKFLKNYRLLHTTLYVTLE | NO. 176 |
| graminum (strain Sg) | PCIMCYGAIIHSRISRLVFGAKYKNLQKYICCKNHFFINKNF | |
| TadA | RKISITQEVLESECSNLLSSFFKRKRKIATKYFNNNII | |
| Rickettsia conorii | MEQALKQAKIAFDKNEVPVGAVVVDRLHQKIIASTHNNTE | SEQ ID |
| TadA | EKNNALYHAEIIAINEACNLISSKNLNDYDIYVTLEPCAMC | NO. 177 |
| | AAAIAHSRLKRLFYGASDSKHGVVESNLRYFNSSACFHRP | |
| | EIYSGILAEDSGLLMKEFFKRIRTVISSHRMT | |
| Staphylococcus | MTNDIYFMTLAIEEAKKAAQLGEVPIGAIITKDDEVIARAH | SEQ ID |
| aureus TadA | NLRETLQQPTAHAEHIAIERAAKVLGSWRLEGCTLYVTLE | NO. 178 |
| | PCVMCAGTIVMSRIPRVVYGADDPKGGCSGSLMNLLQQS | |
| | NFNHRAIVDKGVLKEACSTLLTTFFKNLRANKKSTN | |
| Arabidopsis thaliana | MEEDWGKTVSEKVISAYMSLPKKGKPQGREVTVLSAFLV | SEQ ID |
| ADAT1 | SSPSQDPKVIALGTGTKCVSGSLLSPRGDIVNDSHAEVVAR | NO. 179 |
| | RALIRFFYSEIQRMQLTSGKSNEAKRQRIDSETSSILESADSS | |
| | CPGEVKYKLKSGCLLHLYISQLPCGYASTSSPLYALKKIPS | |
| | TQVDDSLLVQASDICSSRHSDVPEIGSNSNKGNGSQVADM | |
| | VQRKPGRGETTLSVSCSDKIARWNVLGVQGALLYQVLQP | |
| | VYISTITVGQSLHSPDNFSLADHLRRSLYERILPLSDELLTSF | |
| | RLNKPLFFVAPVPPSEFQHSETAQATLTCGYSLCWNYSGL | |
| | HEVILGTTGRKQGTSAKGALYPSTQSSICKQRLLELFLKET | |
| | HGHKRESSKSKKSYRELKNKATEYYLMSKIFKGKYPFNN | |
| | WLRKPLNCEDFLIN | |
| Schizosaccharomyces | MEEFTLDRNSNVGNLIALAVLNKFDELARHGKPIIRANGV | SEQ ID |
| pombe ADAT1 | REWTTLAGVVIQKKMENEFICVCLATGVKCTPAGIIKNEQ | NO. 180 |
| | LGSVLHDCHAEILALRCFNRLLLEHCILIKESKKDTWLLEV | |
| | ADNGKFTLNSNLLIHLYVSECPCGDASMELLASRLENNKP | |
| | WNLTVDSEKLMRGRADFGLLGIVRTKPGRPDAPVSWSKS | |
| | CTDKLAAKQYLSILNSQTSLICEPIYLSCVVLYKKVIVKSAI | |
| | DRAFGPFGRCAPLAEFGEKDNPYYFHPFTVLETDENFLYSR | |
| | PLNQAEKTATSTNVLIWIGDKMQCTQVIHNGIKAGTKAKD | |
| | VEKSQTLICRKSMMNLLHQLSQSLTNEKNYYEWKKLNIK | |
| | RCQQKQILRNILKNWIPNGGNEFQWI | |
| Saccharomyces | MVSCQGTRPCIVNLLTMPSEDKLGEEISTRVINEYSKLKSA | SEQ ID |
| cerevisiae ADAT1 | CRPIIRPSGIREWTILAGVAAINRDGGANKIEILSIATGVKAL | NO. 181 |
| | PDSELQRSEGKILHDCHAEILALRGANTVLLNRIQNYNPSS | |
| | GDKFIQHNDEIPARFNLKENWELALYISRLPCGDASMSFLN | |
| | DNCKNDDFIKIEDSDEFQYVDRSVKTILRGRLNFNRRNVV | |
| | RTKPGRYDSNITLSKSCSDKLLMKQRSSVLNCLNYELFEKP | |
| | VFLKYIVIPNLEDETKHHLEQSFHTRLPNLDNEIKFLNCLKP | |
| | FYDDKLDEEDVPGLMCSVKLFMDDFSTEEAILNGVRNGFY | |
| | TKSSKPLRKHCQSQVSRFAQWELFKKIRPEYEGISYLEFKS | |
| | RQKKRSQLIIAIKNILSPDGWIPTRTDDVK | |
| Macaca fascicularis | MWTADEIALLCYEHYGIRLPKKGKPEPNHEWTLLAAVVKI | SEQ ID |
| ADAT1 | QSPADQDCDTPDKPAQVTKEVVSMGTGTKCIGQSKMRKS | NO. 182 |
| | GDILNDSHAEVIARRNFQRYLLHQLQLAATLKEDSIFVPGT | |
| | QKGLWKLRRDLFFVFFSSHTPCGDASIIPMLEFEDQPCCPVI | |
| | RDWASSSSVEASSNLEAPGNERKCEDLDSPVTKKMRLEPM | |
| | TAAREVTNGATHHQSFGKQESGPISPGINSCNLTVEGLAAV | |
| | TRIAPGSAKVIDVYRTGAKCVPGEAGDSRKPGAAFHQVGL | |
| | LRVKPGRGDRTRSMSCSDKMARWNVLGCQGALLMHFLE | |
| | EPIYLSAVVIGKCPYSQEAMQRALTGRRQNVSALPKGFGV | |
| | QELKILQSDLLFEQSRCAVQAKRADSPGRLVPCGAAISWS | |
| | AVPEQPLDVTANGFPQGTTKKTIGSLQARSQISKVELLRSF | |
| | QKLLSRIARDKWPDSLRVQKLDTYQDYKEAASSYQEAWS | |
| | TLRKQAFGSWIRNPPDYHQFK | |
| Gallus gallus ADAT1 | MWTADEIAELCYEHYRSRLPKQGKPDPSREWTSLAAVVK | SEQ ID |
| | VESAANEAGSAVLGTLQVAKEVVALGTGTKCIGLNKMRK | NO. 183 |
| | TGDVLNDSHAEVVAKRSFQRYLLHQMRLATSYQQCSIFIP | |
| | GTETGKWKLKPNIIFIFFCSHTPCGDASIIPIRETENHLSKSV | |
| | DGHDIAGQSVLCSSSNCDHRGPEDKRKSEKMASSHMIKR | |
| | MKNADGGFFSTITEDMAVQQVFAKPEGNVNPECCESSEE | |
| | MQAANKETNAGKLKAVGVYRTGAKFVPGELSDTLIPGIEY | |
| | HCVGLLRVKPGRGDRTCSMSCSDKLARWNVLGCQGALL | |
| | MHFLQYPVYLSAVIVGKCPYSQEAMQRAVIERCRHISLLP | |
| | DGFLTQEVQLLQSDLQFEHSRQAIQEGQTSSKRKLVPCSA | |
| | AISWSAVPEGPLDVTSDGFRQGTTKKGIGSPQSRSKICKVE | |
| | LFHEFQKLVTSISKENLPDTLRMKTLETYWDYKEAALNYQ | |
| | EAWKALRSQALLGWIKNAQEYLLFM | |
| Human ADAT1 | MWTADEIAQLCYEHYGIRLPKKGKPEPNHEWTLLAAVVKI | SEQ ID |
| | QSPADKACDTPDKPVQVTKEVVSMGTGTKCIGQSKMRKN | NO. 184 |
| | GDILNDSHAEVIARRSFQRYLLHQLQLAATLKEDSIFVPGT | |
| | QKGVWKLRRDLIFVFFSSHTPCGDASIIPMLEFEDQPCCPV | |
| | FRNWAHNSSVEASSNLEAPGNERKCEDPDSPVTKKMRLEP | |
| | GTAAREVTNGAAHHQSFGKQKSGPISPGIHSCDLTVEGLA | |
| | TVTRIAPGSAKVIDVYRTGAKCVPGEAGDSGKPGAAFHQV | |
| | GLLRVKPGRGDRTRSMSCSDKMARWNVLGCQGALLMHL | |
| | LEEPIYLSAVVIGKCPYSQEAMQRALIGRCQNVSALPKGFG | |
| | VQELKILQSDLLFEQSRSAVQAKRADSPGRLVPCGAAISWS | |
| | AVPEQPLDVTANGFPQGTTKKTIGSLQARSQISKVELFRSF | |
| | QKLLSRIARDKWPHSLRVQKLDTYQEYKEAASSYQEAWS | |
| | TLRKQVFGSWIRNPPDYHQFK | |
| Mus musculus | MWTADEIAQLCYAHYNVRLPKQGKPEPNREWTLLAAVV | SEQ ID |
| ADAT1 | KIQASANQACDIPEKEVQVTKEVVSMGTGTKCIGQSKMRE | NO. 185 |
| | SGDILNDSHAEIIARRSFQRYLLHQLHLAAVLKEDSIFVPGT | |
| | QRGLWRLRPDLSFVFFSSHTPCGDASIIPMLEFEEQPCCPVI | |
| | RSWANNSPVQETENLEDSKDKRNCEDPASPVAKKMRLGT | |
| | PARSLSNCVAHHGTQESGPVKPDVSSSDLTKEEPDAANGI | |
| | ASGSFRVVDVYRTGAKCVPGETGDLREPGAAYHQVGLLR | |
| | VKPGRGDRTCSMSCSDKMARWNVLGCQGALLMHFLEKPI | |
| | YLSAVVIGKCPYSQEAMRRALTGRCEETLVLPRGFGVQEL | |
| | EIQQSGLLFEQSRCAVHRKRGDSPGRLVPCGAAISWSAVP | |
| | QQPLDVTANGFPQGTTKKEIGSPRARSRISKVELFRSFQKL | |
| | LSSIADDEQPDSIRVTKKLDTYQEYKDAASAYQEAWGALR | |
| | RIQPFASWIRNPPDYHQFK | |
| Drosophila | MCDNKKPTVKEIAELCLKKFESLPKTGKPTANQWTILAGI | SEQ ID |
| melanogaster | VEFNRNTEACQLVSLGCGTKCIGESKLCPNGLILNDSHAEV | NO. 186 |
| ADAT1 | LARRGFLRFLYQELKQDRIFHWNSTLSTYDMDEHVEFHFL | |
| | STQTPCGDACILEEEQPAARAKRQRLDEDSEMVYTGAKLI | |
| | SDLSDDPMLQTPGALRTKPGRGERTLSMSCSDKIARWNVI | |
| | GVQGALLDVLISKPIYFSSLNFCCDDAQLESLERAIFKRFDC | |
| | RTFKHTRFQPQRPQINIDPGIRFEFSQRSDWQPSPNGLIWSQ | |
| | VPEELRPYEISVNGKRQGVTKKKMKTSQAALAISKYKLFL | |
| | TFLELVKFNPKLSEMFDQQLSDPERIAYASCKDLARDYQF | |
| | AWREIKEKYFLQWTKKPHELLDFNPMSNK | |
| Xenopus tropicalis | MQAKGLWSADEIAALSYGHYTTQLPKQGLPDPSREWTLM | SEQ ID |
| ADAT1 | AAVIQIESVEDTKVIKKVVAMGTGTKCIGQAKLRKTGDVL | NO. 187 |
| | QDSHAEIIAKRSFQRYLLHQLSLAVSDTKDCLFIPGTEKGK | |
| | WMLRPEISFVFFTSHTPCGDASIIPVISHEDELGHPLPSEVTE | |
| | KDHSSNNVCESVNTTYKRKVRSEEDIGFISKKMKHSIDEIL | |
| | TRPENYEEENRHDFPSTCQKALDVHRTGAKCVAGELQDS | |
| | YSPGVNYHTVGVLRIKPGRGDRTMSMSCSDKMARWNVL | |
| | GCQGALLMHFLQQPIYLSAVVVGKCPFSQDAMERALYNR | |
| | CHKVLSLPCAFRLNRVQIIQSDLEFQHGRHALTKKDATRK | |
| | LVPCGAAVSWSAVPHHPLDVTANGYRQGTTRKAIGSPQC | |
| | RSRICKAEIFNTFRELVQRLSEKQRSESLSSQGLKTYWDYK | |
| | AAAITYQEAWNCLRQQAFTSWIQTPRDFLMFS | |
| Dictyostelium | MSWKLDKQFSDKICNFSHDFFNKKLIKKGKPISGEWTVLA | SEQ ID |
| discoideum ADAT1 | TLVLVVENTSSYEIKQVLSLGTGNRCLGKSSLSNQGDVLN | NO. 188 |
| | DSHAEIICKRSFQKFCYNEILNLLQSKYYNSILFNIEYHDSN | |
| | NNNKDNDNNGSLPTISIKKGHSLHFYVNQTPCGDCSIFPFK | |
| | KETQPENFIEKEKLEKDGKDKIENHEKKEQKDIIKQVDKDK | |
| | DEENYEDEESKRKLKKVKDDNNNNNNNNNNNNNNNNNN | |
| | NNNNNNNNNNNNNNNNNNNINNNNNQYDDIQRTGAKTV | |
| | FGEPEDKKLIGVDYHQIGVLRVKPGRGDPTVSMSCSDKIA | |
| | RWNVLGIQGSLLSHFIKEQIFLSSITIGDLFNHSSIYRGLIGR | |
| | LLPNPTTTTTETSSSSSSSSSSSNTIPNFKLNSDLEIFSTNIQF | |
| | QFSKLLLESDQQNNNKSTSSGLAISFCYPNQHEVTIAINGK | |
| | KMGTNQKNFNAISQRSSICKFNLFKLFHQLVLIIKNKNSNE | |
| | ENEKNNQIVLIDSLFNYYECKHLSKKYYQEYEKLKEFKFK | |
| | NWLTNSSDLENFVLDN | |
| Schizosaccharomyces | MAGDSVKSAIIGIAGGPFSGKTQLCEQLLERLKSSAPSTFSK | SEQ ID |
| pombe ADAT2 | LIHLTSFLYPNSVDRYALSSYDIEAFKKVLSLISQGAEKICL | NO. 189 |
| | PDGSCIKLPVDQNRIILIEGYYLLLPELLPYYTSKIFVYEDA | |
| | DTRLERCVLQRVKAEKGDLTKVLNDFVTLSKPAYDSSIHP | |
| | TRENADIILPQKENIDTALLFVSQHLQDILAEMNKTSSSNTV | |
| | KYDTQHETYMKLAHEILNLGPYFVIQPRSPGSCVFVYKGE | |
| | VIGRGFNETNCSLSGIRHAELIAIEKILEHYPASVFKETTLY | |
| | VTVEPCLMCAAALKQLHIKAVYFGCGNDRFGGCGSVFSIN | |
| | KDQSIDPSYPVYPGLFYSEAVMLMREFYVQENVKAPVPQS | |
| | KKQRVLKREVKSLDLSRFK | |
| Saccharomyces | MQHIKHMRTAVRLARYALDHDETPVACIFVHTPTGQVMA | SEQ ID |
| cerevisiae ADAT2 | YGMNDTNKSLTGVAHAEFMGIDQIKAMLGSRGVVDVFK | NO. 190 |
| | DITLYVTVEPCIMCASALKQLDIGKVVFGCGNERFGGNGT | |
| | VLSVNHDTCTLVPKNNSAAGYESIPGILRKEAIMLLRYFYV | |
| | RQNERAPKPRSKSDRVLDKNTFPPMEWSKYLNEEAFIETF | |
| | GDDYRTCFANKVDLSSNSVDWDLIDSHQDNIIQELEEQCK | |
| | MFKFNVHKKSKV | |
| Xenopus tropicalis | MTEEIQNWMHKAFQMAQDALNNGEVPVGCLMVYDNQV | SEQ ID |
| ADAT2 | VGKGRNEVNETKNATRHAEMVAIDQVLDWCEKNSKKSR | NO. 191 |
| | DVFENIVLYVTVEPCIMCAGALRLLKIPLVVYGCRNERFG | |
| | GCGSVLNVAGDNIPDTGTEFKYIGGYQAEKAVELLKTFYK | |
| | QENPNAPRSKVRKKE | |
| Bos taurus ADAT2 | MEAKAGPTAATDGAYSVSAEETEKWMEQAMQMAKDAL | SEQ ID |
| | DNTEVPVGCLMVYNNEVVGKGRNEVNQTKNATRHAEMV | NO. 192 |
| | AIDQALDWCRRRGRSPSEVFEHTVLYVTVEPCIMCAAALR | |
| | LMRIPLVVYGCQNERFGGCGSVLDIASADLPSTGKPFQCTP | |
| | GYRAEEAVEMLKTFYKQENPNAPKSKVRKKECHKS | |
| Danio rerio ADAT2 | MQEVGVDPEKNDFLQPSDSEVQTWMAKAFDMAVEALEN | SEQ ID |
| | GEVPVGCLMVYNNEIIGKGRNEVNETKNATRHAEMVALD | NO. 193 |
| | QVLDWCRLREKDCKEVCEQTVLYVTVEPCIMCAAALRLL | |
| | RIPFVVYGCKNERFGGCGSVLDVSSDHLPHTGTSFKCIAGY | |
| | RAEEAVEMLKTFYKQENPNAPKPKVRKDSINPQDGAAVIQ | |
| | VMRGPPDEETETIAHLS | |
| Arabidopsis thaliana | MEEDHCEDSHNYMGFALHQAKLALEALEVPVGCVFLEDG | SEQ ID |
| ADAT2 | KVIASGRNRTNETRNATRHAEMEAIDQLVGQWQKDGLSP | NO. 194 |
| | SQVAEKFSKCVLYVTCEPCIMCASALSFLGIKEVYYGCPN | |
| | DKFGGCGSILSLHLGSEEAQRGKGYKCRGGIMAEEAVSLF | |
| | KCFYEQGNPNAPKPHRPVVQRERT | |
| Mus musculus | MEEKVESTTTPDGPCVVSVQETEKWMEEAMRMAKEALE | SEQ ID |
| ADAT2 | NIEVPVGCLMVYNNEVVGKGRNEVNQTKNATRHAEMVA | NO. 195 |
| | IDQVLDWCHQHGQSPSTVFEHTVLYVTVEPCIMCAAALRL | |
| | MKIPLVVYGCQNERFGGCGSVLNIASADLPNTGRPFQCIPG | |
| | YRAEEAVELLKTFYKQENPNAPKSKVRKKDCQKS | |
| Human ADAT2 | MEAKAAPKPAASGACSVSAEETEKWMEEAMHMAKEALE | SEQ ID |
| | NTEVPVGCLMVYNNEVVGKGRNEVNQTKNATRHAEMVA | NO. 196 |
| | IDQVLDWCRQSGKSPSEVFEHTVLYVTVEPCIMCAAALRL | |
| | MKIPLVVYGCQNERFGGCGSVLNIASADLPNTGRPFQCIPG | |
| | YRAEEAVEMLKTFYKQENPNAPKSKVRKKECQKS | |
| Arabidopsis thaliana | MDSDAWEIIHIPEKPSLSPDHQPTVKVYASLIKPRFANTIVR | SEQ ID |
| ADAT3 | HLCKIAPLEDLRHVKRVKKKILPDCGETQLTVILCLAPEHN | NO. 197 |
| | DQLSDMPPDVQRLVDPYELSPFITQVCKYAAVSKEEWEEQ | |
| | SKIWPTSFHPPTYNIDGIGGFSEEETQSICKFMRVVIDMAVS | |
| | GHTPLVNAAVIVDPSVRRIIASETDQVYASSAPRDMTSAET | |
| | RPFEETGEICLNDTLEKQNGSLSALSCLNPWQWSLQPHDT | |
| | ENCSQWHPLRHASMVAIESSSARDRNLFPNPSKIFDQDHVP | |
| | PSNTDSPAKKQKTSSQSPDVQNDSREETVRDPSMERPYLC | |
| | TGYDIFLLLEPCTMCAMALVHQRIKRIFYAFPNTTAGGLGS | |
| | VHRLQGEKSLNHHYAVFRVLLPDDALRQMTTV | |
| Mus musculus | MEPTSGFAEQPGPVKAESEEQEPAQWQALPVLSEQQSGAV | SEQ ID |
| ADAT3 | ELILAYAAPVLDKRQTSRLLREVSAVYPLPAQPHLKRVRPS | NO. 198 |
| | RSAGGAQSSDLLLCLAGPSAGPRSLAELLPRPAVDPRGLGT | |
| | PFLVPVPARPPLTRSQFEEARAHWPTSFHEDKQVTSALAG | |
| | QLFSTQERAAMQTHMERAVCAAQRAAAQGLRAVGAVVV | |
| | DPASDRVLATGHDCSSVASPLLHAVMVCIDLVAQGQGRG | |
| | SCDLRSHPACSFTQATATQGARAGSVRKLDEDSLPYVCTG | |
| | YDLYVTREPCVMCAMALVHARIQRVFYGAPSPDGALGTL | |
| | FRVHARPDLNHRFQVFRGILEDQCRQLDPDP | |
| Danio rerio ADAT3 | MEPQAKRKKEMDDYDDTWEVLPVLSDEQSQDPELLPAYA | SEQ ID |
| | APILERRETSRLVKELSLIHPLPNLQHIKRVRPCKHKDSPHP | NO. 199 |
| | LEVIVCLVSDVQCTDPKKVTLSHLLHTQCFNSNGLGDPFIV | |
| | QIPANPPLTRPQFEKASKHWPTSFHEDKLVTFALKGOLFTA | |
| | HQKTKMREYMCVAVKAAKSGRELGMDAVGAVIVDPKTE | |
| | QIIAVAHDCKRGSHPLHHAVMVCIDLVACGQDGGAYNYE | |
| | KYPACRFSCSNSVCDGKETGLPYICTGYDLYVTREPCVMC | |
| | AMALVHSRISRVFYGASTADGAFGSRYKIHCQKDLNHRFE | |
| | VFKGVMVNACEDLCKE | |
| Schizosaccharomyces | MVKTNISKNSPKEATVPELDWPFKLIKSHLETRKLETENV | SEQ ID |
| pombe ADAT3 | WIACFEPKYASKVTQYVKQIRSKQKESLLHCNRLRRIQDE | NO. 200 |
| | NGSLELQIIICPEKSMTANEIGKDFEDLGIVSKMIFLYAVPA | |
| | FPPLTDEQFHEWNSVWPVSYRKHVQRQDVFTVHELKRIES | |
| | ILEDLINAAGASHKHGEIGCAAAIYDPTTDTVLAVSVDERS | |
| | KLKNPINHCVMNAINLVAKRELSRRQNRTDGSKDRYLCK | |
| | DLTVVMTHEPCVMCSMGLLHSRIRRLIYCKKQPLTGGIESL | |
| | YGIHWRAELNHRYLAYSGWNKPVPSIKENIHV | |
| Saccharomyces | MVKKVNNPLKIDYQNGIIENRLLQIRNFKDVNTPKLINVW | SEQ ID |
| cerevisiae ADAT3 | SIRIDPRDSKKVIELIRNDFQKNDPVSLRHLKRIRKDIETSTL | NO. 201 |
| | EVVLCSKEYICDEGEINNKLKSIWVGTKKYELSDDIEVPEF | |
| | APSTKELNNAWSVKYWPLIWNGNPNDQILNDYKIDMQEV | |
| | RNELSRASTLSVKMATAGKQFPMVSVFVDPSRKKDKVVA | |
| | EDGRNCENSLPIDHSVMVGIRAVGERLREGVDEDANSYLC | |
| | LDYDVYLTHEPCSMCSMALIHSRVRRVVFLTEMQRTGSLK | |
| | LTSGDGYCMNDNKQLNSTYEAFQWIGEEYPVGQVDRDVC | |
| | C | |
| Human ADAT3 | MEPAPGLVEQPKCLEAGSPEPEPAPWQALPVLSEKQSGDV | SEQ ID |
| | ELVLAYAAPVLDKRQTSRLLKEVSALHPLPAQPHLKRVRP | NO. 202 |
| | SRDAGSPHALEMLLCLAGPASGPRSLAELLPRPAVDPRGL | |
| | GQPFLVPVPARPPLTRGQFEEARAHWPTSFHEDKQVTSAL | |
| | AGRLFSTQERAAMQSHMERAVWAARRAAARGLRAVGAV | |
| | VVDPASDRVLATGHDCSCADNPLLHAVMVCVDLVARGQ | |
| | GRGTYDFRPFPACSFAPAAAPQAVRAGAVRKLDADEDGL | |
| | PYLCTGYDLYVTREPCAMCAMALVHARILRVFYGAPSPD | |
| | GALGTRFRIHARPDLNHRFQVFRGVLEEQCRWLDPDT | |
| Rattus norvegicus | MEPTSGFAEQPGPEKVESEEQEPAQWQALPVLSEQQSGAV | SEQ ID |
| ADAT3 | ELVLAYAAPVLDKRQTSRLLREVSAVYPLPAQPHLKRVRP | NO. 203 |
| | SRSAGGAHSSDLLLCLAGPSAGPRSLAELLPRPAVDPRGLG | |
| | TPFLVPVPARPPLTRSQFEEARAHWPTSFHEDKQVTSALAG | |
| | QLFSAQARAAMQTHMERAVRAAQRAAAQGLRAVGAVV | |
| | VDPASDHVLATGHDCCSEASPLLHAVMVCIDLVAQGQGR | |
| | GSCDLRRHPACSFTQATATQSARAGSVRKLDEDSLPYVCT | |
| | GYDLYVTREPCVMCAMALVHARIQRVFYGAPSPDGALGT | |
| | RFRVHARPDLNHRFQVFRGILEDQCRQLDPDP | |
| Rattus norvegicus | MDIEDEENMSSSSIDVKENRNLDNMPPKDSSTPGPGEGIPL | SEQ ID |
| ADARBI | SNGGGGSTSRKRPLEEGSNGHSKYRLKKRRKTPGPVLPKN | NO. 204 |
| | ALMQLNEIKPGLQYMLLSQTGPVHAPLFVMSVEVNGQVF | |
| | EGSGPTKKKAKLHAAEKALRSFVQFPNASEAHLAMGRTL | |
| | SVNTDFTSDQADFPDTLFNGFETPDKSEPPFYVGSNGDDSF | |
| | SSSGDVSLSASPVPASLTQPPLPIPPPFPPPSGKNPVMILNEL | |
| | RPGLKYDFLSESGESHAKSFVMSVVVDGQFFEGSGRNKKL | |
| | AKARAAQSALATVFNLHLDQTPSRQPVLSEGLQLHLPQVL | |
| | ADAVSRLVLGKFSDLTDNFSSPHARRKVLSGVVMTTGTD | |
| | VKDAKVISVSTGTKCINGEYMSDRGLALNDCHAEIISRRSL | |
| | LRFLYAQLELYLNNKEDQKKSIFQKSERGGFRLKDTVQFH | |
| | LYISTSPCGDARIFSPHEPVLEGMAPDSHQLTEPADRHPNR | |
| | KARGQLRTKIESGEGTIPVRSNASIQTWDGVLQGERLLTMS | |
| | CSDKIARWNVVGIQGALLSIFVEPIYFSSIILGSLYHGDHLS | |
| | RAMYQRISNIEDLPPLYTLNKPLLSGISNAEARQPGKAPNF | |
| | SVNWTVGDTAIEVINATTGKDELGRPSRLCKHALYCRWM | |
| | RVHGKVPPHLLRTKITKPTTYHESKLAAKEYQAAKARLFT | |
| | AFIKAGLGAWVEKPTEQDQFSFTP | |
| Human ADAR | MNPRQGYSLSGYYTHPFQGYEHRQLRYQQPGPGSSPSSFL | SEQ ID |
| | LKQIEFLKGQLPEAPVIGKQTPSLPPSLPGLRPRFPVLLASST | NO. 205 |
| | RGRQVDIRGVPRGVHLRSQGLQRGFQHPSPRGRSLPQRGV | |
| | DCLSSHFQELSIYQDQEQRILKFLEELGEGKATTAHDLSGK | |
| | LGTPKKEINRVLYSLAKKGKLQKEAGTPPLWKIAVSTQAW | |
| | NQHSGVVRPDGHSQGAPNSDPSLEPEDRNSTSVSEDLLEPF | |
| | IAVSAQAWNQHSGVVRPDSHSQGSPNSDPGLEPEDSNSTS | |
| | ALEDPLEFLDMAEIKEKICDYLFNVSDSSALNLAKNIGLTK | |
| | ARDINAVLIDMERQGDVYRQGTTPPIWHLTDKKRERMQIK | |
| | RNTNSVPETAPAAIPETKRNAEFLTCNIPTSNASNNMVTTE | |
| | KVENGQEPVIKLENRQEARPEPARLKPPVHYNGPSKAGYV | |
| | DFENGQWATDDIPDDLNSIRAAPGEFRAIMEMPSFYSHGLP | |
| | RCSPYKKLTECQLKNPISGLLEYAQFASQTCEFNMIEQSGP | |
| | PHEPRFKFQVVINGREFPPAEAGSKKVAKQDAAMKAMTIL | |
| | LEEAKAKDSGKSEESSHYSTEKESEKTAESQTPTPSATSFFS | |
| | GKSPVTTLLECMHKLGNSCEFRLLSKEGPAHEPKFQYCVA | |
| | VGAQTFPSVSAPSKKVAKQMAAEEAMKALHGEATNSMA | |
| | SDNQPEGMISESLDNLESMMPNKVRKIGELVRYLNTNPVG | |
| | GLLEYARSHGFAAEFKLVDQSGPPHEPKFVYQAKVGGRW | |
| | FPAVCAHSKKQGKQEAADAALRVLIGENEKAERMGFTEV | |
| | TPVTGASLRRTMLLLSRSPEAQPKTLPLTGSTFHDQIAMLS | |
| | HRCFNTLTNSFQPSLLGRKILAAIIMKKDSEDMGVVVSLGT | |
| | GNRCVKGDSLSLKGETVNDCHAEIISRRGFIRFLYSELMKY | |
| | NSQTAKDSIFEPAKGGEKLQIKKTVSFHLYISTAPCGDGAL | |
| | FDKSCSDRAMESTESRHYPVFENPKQGKLRTKVENGEGTI | |
| | PVESSDIVPTWDGIRLGERLRTMSCSDKILRWNVLGLQGA | |
| | LLTHFLQPIYLKSVTLGYLFSQGHLTRAICCRVTRDGSAFE | |
| | DGLRHPFIVNHPKVGRVSIYDSKRQSGKTKETSVNWCLAD | |
| | GYDLEILDGTRGTVDGPRNELSRVSKKNIFLLFKKLCSFRY | |
| | RRDLLRLSYGEAKKAARDYETAKNYFKKGLKDMGYGNW | |
| | ISKPQEEKNFYLCPV | |
| Rattus norvegicus | MSQGFRGPTGVFPHQTQPCLDPSYEHSKWRYLQPRGSESY | SEQ ID |
| ADAR | LRSFQLQQIEFLKGRLPEAPLIGAQTQSLPPFLPGHWPRFPG | NO. 206 |
| | PPAQGKQPEIWGFPRSVTLRNQGFHIGPPLPPPHSRGPPWR | |
| | GAEGLCSHFQELSISQNPEQKVLNRLEELGEGKATTAYAL | |
| | ARELRTPKKDINRILYSLERKGKLHRGVGKPPLWSLVPLSQ | |
| | ACTQPPRAVNSDKEVPRGEPDLDSEDGDPASDLEGPSELL | |
| | DMAEIKEKICDYLFNVSKSSALNLAKNIGLAKARDVNAVL | |
| | IDLERQGDVYREGATPPIWYLTDKKRERLQMKRSTHSGPA | |
| | ATPAAVSEATQSTSFPTCHPPQSGGSSSMATSKRVENGQEP | |
| | VTKYESRHEARPGPVRLRPHAYHNGPSRAGYVASENGPW | |
| | ATDDIPDNLNSIHTAPGEFRAIMEMPSFYSPTLPRCSPYKKL | |
| | TECQLKNPVSGLLEYAQFTSQTCDFNLIEQSGPSHEPRFKF | |
| | QVVINGREFPPAEAGSKKVAKQDAAVKAMAILLREAKAK | |
| | DSGQPEELSNCPMEEDPEKPAESQPPSSSATSLFSGKSPVTT | |
| | LLECMHKLGNSCEFRLLSKEGPAHDPKFQYCVAVGAQTFP | |
| | SVSAPSKKVAKQMAAEEAMKALQEEAANSADDQSGGAN | |
| | TDSLDESVAPNKIRRIGELVRYLNTNPVGGLLEYARSHGFA | |
| | AEFKLIDQSGPPHEPKFVYQAKVGGRWFPAVCAHSKKQG | |
| | KQDAADAALRVLIGESEKAEQLGFAEVTPVTGASLRRTML | |
| | LLSRSPDAHPKTLPLTGSTFHDQIAMLSHRCFNALTNSFQP | |
| | SLLGRKILAAIIMKRDPEDMGVVVSLGTGNRCVKGDSLSL | |
| | KGETVNDCHAEIISRRGFIRFLYSELMKYNHHTAKNSIFEL | |
| | ARGGEKLQIKKTVSFHLYISTAPCGDGAHFDKSCSDRAVE | |
| | STESRHYPVFENPKQGKLRTKVENGEGTIPVESSDIVPTWD | |
| | GIRLGERLRTMSCSDKILRWNVLGLQGALLTHFLQPVYLK | |
| | SVTLGYLFSQGHLTRAICCRVTRDGNAFEDGLRYPFIVNHP | |
| | KVGRVSVYDSKRQSGKTKETSVNWCLADGYDLEILDGTR | |
| | GTVDGPGKELSRVSKKNIFLQFKKLCSFRARRDLLQLSYG | |
| | EAKKAARDYDLAKNYFKKSLRDMGYGNWISKPQEEKNF | |
| | YLCPVPND | |
| Human ADARBI | MDIEDEENMSSSSTDVKENRNLDNVSPKDGSTPGPGEGSQ | SEQ ID |
| | LSNGGGGGPGRKRPLEEGSNGHSKYRLKKRRKTPGPVLPK | NO. 207 |
| | NALMQLNEIKPGLQYTLLSQTGPVHAPLFVMSVEVNGQVF | |
| | EGSGPTKKKAKLHAAEKALRSFVQFPNASEAHLAMGRTL | |
| | SVNTDFTSDQADFPDTLFNGFETPDKAEPPFYVGSNGDDSF | |
| | SSSGDLSLSASPVPASLAQPPLPVLPPFPPPSGKNPVMILNE | |
| | LRPGLKYDELSESGESHAKSFVMSVVVDGQFFEGSGRNKK | |
| | LAKARAAQSALAAIFNLHLDQTPSRQPIPSEGLQLHLPQVL | |
| | ADAVSRLVLGKFGDLTDNFSSPHARRKVLAGVVMTTGTD | |
| | VKDAKVISVSTGTKCINGEYMSDRGLALNDCHAEIISRRSL | |
| | LRFLYTQLELYLNNKDDQKRSIFQKSERGGFRLKENVQFH | |
| | LYISTSPCGDARIFSPHEPILEGSRSYTQAGVQWCNHGSLQP | |
| | RPPGLLSDPSTSTFQGAGTTEPADRHPNRKARGQLRTKIES | |
| | GEGTIPVRSNASIQTWDGVLQGERLLTMSCSDKIARWNVV | |
| | GIQGSLLSIFVEPIYFSSIILGSLYHGDHLSRAMYQRISNIEDL | |
| | PPLYTLNKPLLSGISNAEARQPGKAPNFSVNWTVGDSAIEV | |
| | INATTGKDELGRASRLCKHALYCRWMRVHGKVPSHLLRS | |
| | KITKPNVYHESKLAAKEYQAAKARLFTAFIKAGLGAWVE | |
| | KPTEQDQFSLTP | |
| Mus musculus | MDIEDEENMSSSSTDIKENRNLDNMPPKDSSTPGPGEGIPL | SEQ ID |
| ADARBI | SNGGGGSTSRKRPLEEGSNGHSKYRLKKRRKTPGPVLPKN | NO. 208 |
| | ALMQLNEIKPGLQYMLLSQTGPVHAPLFVMSVEVNGQVF | |
| | EGSGPTKKKAKLHAAEKALRSFVQFPNASEAHLAMGRTL | |
| | SVNTDFTSDQADFPDTLFNGFETPDKSEPPFYVGSNGDDSF | |
| | SSSGDVSLSASPVPASLTQPPLPIPPPFPPPSGKNPVMILNEL | |
| | RPGLKYDFLSESGESHAKSFVMSVVVDGQFFEGSGRNKKL | |
| | AKARAAQSALATVFNLHLDQTPSRQPVLSEGLQLHLPQVL | |
| | ADAVSRLVLGKFSDLTDNFSSPHARRKVLSGVVMTTGTD | |
| | VKDAKVISVSTGTKCINGEYMSDRGLALNDCHAEIISRRSL | |
| | LRFLYAQLELYLNNKEDQKKSIFQKSERGGFRLKDTVQFH | |
| | LYISTSPCGDARIFSPHEPVLEGMTPDSHQLTEPADRHPNR | |
| | KARGQLRTKIESGEGTIPVRSNASIQTWDGVLQGERLLTMS | |
| | CSDKIARWNVVGIQGSLLSIFVEPIYFSSIILGSLYHGDHLSR | |
| | AMYQRISNIEDLPPLYTLNKPLLSGISNAEARQPGKAPNFS | |
| | VNWTVGDATIEVINATTGKDELGRPSRLCKHALYCRWMR | |
| | VHGKVPPHLLRTKITKPTTYHESKLAAREYQAAKARLFTA | |
| | FIKAGLGAWVEKPTEQDQFSFTP | |
| Mus musculus | MSQGFRGPTGVFPHQTQSYLDPSHEHSKWRYPQPQGPESY | SEQ ID |
| ADAR | PRSFQLQQIEFLKGRLPEAPLIGIQTQSLPPFLPGHWPRFPGP | NO. 209 |
| | PAQDRQLEIWEFPRSVTLRNQGFHIGPPLPPPHSRGTPWRG | |
| | ADGLCSHFRELSISQSPEQKVLNRLEELGEGKATTAHVLAR | |
| | ELRIPKRDINRILYSLEKKGKLHRGRGKPPLWSLVPLSQAW | |
| | TQPPGVVNPDSCIQEFPRGEPGLDSEDGDPASDLEGPSEPL | |
| | DMAEIKEKICDYLFNVSNSSALNLAKNIGLTKARDVTSVLI | |
| | DLERQGDVYRQGATPPIWYLTDKKRERLQMKRSTHSAPA | |
| | PTPTAVPEATRSPSFPACHPPPAGASSSVAASKRVENGQEP | |
| | AIKHESRHEARPGPMRLRPHAYHNGPSRAGYVASENGQW | |
| | ATDDIPDNLNSIHTAPGEFRAIMEMPSFYSPTLPRCSPYKKL | |
| | TECQLKNPVSGLLEYAQFTSQTCDFNLIEQSGPSHEPRFKF | |
| | QVVINGREFPPAEAGSKKVAKQDAAVKAMAILLREAKAK | |
| | DSGQPEDLSHCPMEEDSEKPAEAQAPSSSATSLFSGKSPVT | |
| | TLLECMHKLGNSCEFRLLSKEGPAHDPKFQYCVAVGAQTF | |
| | PPVSAPSKKVAKQMAAEEAMKALQEEAASSADDQSGGA | |
| | NTDSLDESMAPNKIRRIGELVRYLNTNPVGGLLEYARSHG | |
| | FAAEFKLIDQSGPPHEPKFVYQAKVGGRWFPAVCAHSKK | |
| | QGKQDAADAALRVLIGESEKAEQLGFAEVTPVTGASLRRT | |
| | MLLLSRSPDAHPKTLPLSGSTFHDQIAMLSHRCFNALTNSF | |
| | QPSLLGRKILAAIIMKRDPEDMGVVVSLGTGNRCVKGDSL | |
| | SLKGETVNDCHAEIISRRGFIRFLYSELMKYNHHTAKNSIFE | |
| | LARGGEKLQIKKTVSFHLYISTAPCGDGALFDKSCSDRAVE | |
| | STESRHYPVFENPKQGKLRTKVENGEGTIPVESSDIVPTWD | |
| | GIRLGERLRTMSCSDKILRWNVLGLQGALLTHFLQPVYLK | |
| | SVTLGYLFSQGHLTRAICCRVTRDGKAFEDGLRYPFIVNHP | |
| | KVGRVSVYDSKRQSGKTKETSVNWCMADGYDLEILDGTR | |
| | GTVDGPGKELSRVSKKNIFLQFKKLCSFRARRDLLQLSYG | |
| | EAKKAARDYDLAKNYFKKSLRDMGYGNWISKPQEEKNF | |
| | YLCPVPND | |
| Human ADARB2- | MKQLFPPPPGTSLTHALGAWRGRERAQAATSLLASSASQF | SEQ ID |
| AS1 | PTAVEDALMSVLTSHCAPSTPAATRAQQTGTRGHIHPACP | NO. 210 |
| | CQQSCVGASRPPGRPQIFLPLTTALSLEAYAADTCSAADFL | |
| | HNPSSWGKVWYLNEASFDLYSYHYFW | |
| Rattus norvegicus | MASVLGSGRGSGGLSSQLKCKSKRRRRRRSKRKDKVSILS | SEQ ID |
| ADARB2 | TFLAPFKYLSPGTTNTEDEDNLSTSSAEVKENRNVSNLGTR | NO. 211 |
| | PLPPGDWARGGSTPSVKRKRPLEEGNGGHFCKLQLIWKKL | |
| | SWSMTPKNALVQLHELKPGLQYRMVSQTGPVHAPVFAVA | |
| | VEVNGLTFEGTGPTKKKAKMRAAEMALKSFVQFPNAFQA | |
| | HLAMGSSTSPCTDFTSDQADFPDTLFKEFEPSSRNEDFPGC | |
| | CPVDTEFLSSAYRRGRLLYHTLDLMGQALPDRSRLAPGAL | |
| | GERNPVVVLNELRSGLRYVCLSETAEKPRVKSFVMAVCV | |
| | DGRTFEGSGRSKKLAKGQAAQAALQALFDIRLPGHIPSRS | |
| | KSNLLPQDFADSVSQLVTQKFRELTVGLTSVYARHKTLAG | |
| | IVMTKGLDTKQAQVIVLSSGTKCISGEHISDQGLVVNDCH | |
| | AEIVARRAFLHFLYTQLELHLSKHQEDPERSIFIRVKEGGY | |
| | RLRENILFHLYVSTSPCGDARLNSPYEITIDLNSSKHIVRKF | |
| | RGHLRTKIESGEGTVPVRGPSAVQTWDGILLGEQLVTMSC | |
| | TDKIASWNVLGLQGALLCHFIEPVYLHSIIVGSLHHTGHLA | |
| | RVMSHRMEGIGQLPASYRQNRPLLSGVSNAEARQPGKSPH | |
| | FSANWVVGSADLEIINATTGKRSCGGSSRLCKHVFSARWA | |
| | RLHGRLSTRIPGHGDTPSMYCEAKRGAHTYQSVKQQLFK | |
| | AFQKAGLGTWVRKPPEQDQFLLSL | |
| Mus musculus | MASVLGSGRGSGGLSSQLKCKSKRRRRRRSKRKDKVSILS | SEQ ID |
| ADARB2 | TFLAPFKYLSPGTTNTEDEDNLSTSSAEVKENRNVSNLGTR | NO. 212 |
| | PLPPGDWARGSTPSVKRKRPLEEGNGGHFCKLQLIWKKLS | |
| | WSVTPKNALVQLHELKPGLQYRMVSQTGPVHAPVFAVAV | |
| | EVNGLTFEGTGPTKKKAKMRAAEMALKSFVQFPNAFQAH | |
| | LAMGSSTSPCTDFTSDQADFPDTLFKEFEPSSKNEDFPGCH | |
| | PVDTEFLSSAYRRGRLLYHTLDLMGQALPDRSRLAPGALG | |
| | ERNPVVVLNELRSGLRYVCLSETAEKPRVKSFVMAVCVD | |
| | GRTFEGSGRSKKLAKGQAAQAALQALFDIRLPGHIPSRSKS | |
| | NLLPQDFADSVSQLVTQKFRELTVGLTSVYARHKTLAGIV | |
| | MTKGLDTKQAQVIVLSSGTKCISGEHISDQGLVVNDCHAEI | |
| | VARRAFLHFLYSQLELHLSKHQEDPERSIFIRLKEGGYRLR | |
| | ENILFHLYVSTSPCGDARVNSPYEITTDLNSSKHIVRKFRGH | |
| | LRTKIESGEGTVPVRGPSAVQTWDGILLGEQLITMSCTDKI | |
| | ASWNVLGLQGALLCHFIEPVYLHSIIVGSLHHTGHLARVM | |
| | SHRMEGIGQLPASYRQNRPLLSGVSHAEARQPGKSPHFSA | |
| | NWVVGSADLEIINATTGKRSCGGSSRLCKHVFSAWWARL | |
| | HGRLSTRIPSHGDTPSMYCEAKQGAHTYQSVKQQLFKAFQ | |
| | KAGLGTWVRKPPEQDQFLLSL | |
| Human ADARB2 | MASVLGSGRGSGGLSSQLKCKSKRRRRRRSKRKDKVSILS | SEQ ID |
| | TFLAPFKHLSPGITNTEDDDTLSTSSAEVKENRNVGNLAAR | NO. 213 |
| | PPPSGDRARGGAPGAKRKRPLEEGNGGHLCKLQLVWKKL | |
| | SWSVAPKNALVQLHELRPGLQYRTVSQTGPVHAPVFAVA | |
| | VEVNGLTFEGTGPTKKKAKMRAAELALRSFVQFPNACQA | |
| | HLAMGGGPGPGTDFTSDQADFPDTLFQEFEPPAPRPGLAG | |
| | GRPGDAALLSAAYGRRRLLCRALDLVGPTPATPAAPGERN | |
| | PVVLLNRLRAGLRYVCLAEPAERRARSFVMAVSVDGRTF | |
| | EGSGRSKKLARGQAAQAALQELFDIQMPGHAPGRARRTP | |
| | MPQEFADSISQLVTQKFREVTTDLTPMHARHKALAGIVMT | |
| | KGLDARQAQVVALSSGTKCISGEHLSDQGLVVNDCHAEV | |
| | VARRAFLHFLYTQLELHLSKRREDSERSIFVRLKEGGYRLR | |
| | ENILFHLYVSTSPCGDARLHSPYEITTDLHSSKHLVRKFRG | |
| | HLRTKIESGEGTVPVRGPSAVQTWDGVLLGEQLITMSCTD | |
| | KIARWNVLGLQGALLSHFVEPVYLQSIVVGSLHHTGHLAR | |
| | VMSHRMEGVGQLPASYRHNRPLLSGVSDAEARQPGKSPP | |
| | FSMNWVVGSADLEIINATTGRRSCGGPSRLCKHVLSARWA | |
| | RLYGRLSTRTPSPGDTPSMYCEAKLGAHTYQSVKQQLFKA | |
| | FQKAGLGTWVRKPPEQQQFLLTL | |
| Rattus norvegicus | MPLFKLTGQGKQIDDAMRSFAEKVFASEVKDEGGRHEISP | SEQ ID |
| AMPD1 | FDVDEICPISLREMQAHIFHMENLSMSMDGRRKRRFQGRK | NO. 214 |
| | TVNLSIPQSETSSTKLSHIEEFISSSPTYESVPDFQRVQITGD | |
| | YASGVTVEDFEVVCKGLYRALCIREKYMQKSFQRFPKTPS | |
| | KYLRNIDGEALVAIESFYPVFTPPPKKGEDPFRREDLPANL | |
| | GYHLKMKGGVIYIYPDEAAASRDEPKPYPYPNLDDFLDD | |
| | MNFLLALIAQGPVKTYTHRRLKFLSSKFQVHQMLNEMDE | |
| | LKELKNNPHRDFYNCRKVDTHIHAAACMNQKHLLRFIKK | |
| | SYHIDADRVVYSTKEKNLTLKELFAQLNMHPYDLTVDSLD | |
| | VHAGRQTFQRFDKFNDKYNPVGASELRDLYLKTDNYING | |
| | EYFATIIKEVGADLVDAKYQHAEPRLSIYGRSPDEWSKLSS | |
| | WFVGNRIYCPNMTWMIQVPRIYDVFRSKNFLPHFGKMLE | |
| | NIFLPVFEATINPQTHPDLSVFLKHITGFDSVDDESKHSGH | |
| | MFSSKSPKPEEWTMENNPSYTYYAYYMYANIMVLNCLRK | |
| | ERGMNTFLFRPHCGEAGALTHLMTAFMIADNISHGLNLKK | |
| | SPVLQYLFFLAQIPIAMSPLSNNSLFLEYAKNPFLDFLQKGL | |
| | MISLSTDDPMQFHFTKEPLMEEYAIAAQVFKLSTCDMCEV | |
| | ARNSVLQCGISHEEKAKFLGNNYLEEGPVGNDIRRTNVAQ | |
| | IRMAYRYETWCYELNLIAEGLKSTE | |
| Human AMPD1 | MPLFKLPAEEKQIDDAMRNFAEKVFASEVKDEGGRQEISP | SEQ ID |
| | FDVDEICPISHHEMQAHIFHLETLSTSTEARRKKRFQGRKT | NO. 215 |
| | VNLSIPLSETSSTKLSHIDEYISSSPTYQTVPDFQRVQITGDY | |
| | ASGVTVEDFEIVCKGLYRALCIREKYMQKSFQRFPKTPSK | |
| | YLRNIDGEAWVANESFYPVFTPPVKKGEDPFRTDNLPENL | |
| | GYHLKMKDGVVYVYPNEAAVSKDEPKPLPYPNLDTFLDD | |
| | MNFLLALIAQGPVKTYTHRRLKFLSSKFQVHQMLNEMDE | |
| | LKELKNNPHRDFYNCRKVDTHIHAAACMNQKHLLRFIKK | |
| | SYQIDADRVVYSTKEKNLTLKELFAKLKMHPYDLTVDSLD | |
| | VHAGRQTFQRFDKFNDKYNPVGASELRDLYLKTDNYING | |
| | EYFATIIKEVGADLVEAKYQHAEPRLSIYGRSPDEWSKLSS | |
| | WFVCNRIHCPNMTWMIQVPRIYDVFRSKNFLPHFGKMLE | |
| | NIFMPVFEATINPQADPELSVFLKHITGFDSVDDESKHSGH | |
| | MFSSKSPKPQEWTLEKNPSYTYYAYYMYANIMVLNSLRK | |
| | ERGMNTFLFRPHCGEAGALTHLMTAFMIADDISHGLNLKK | |
| | SPVLQYLFFLAQIPIAMSPLSNNSLFLEYAKNPFLDFLQKGL | |
| | MISLSTDDPMQFHFTKEPLMEEYAIAAQVFKLSTCDMCEV | |
| | ARNSVLQCGISHEEKVKFLGDNYLEEGPAGNDIRRTNVAQ | |
| | IRMAYRYETWCYELNLIAEGLKSTE | |
| Oryctolagus | MNQKHLLRFIKKSYQVDADRVVYSTK | SEQ ID |
| cuniculus AMPD1 | | NO. 216 |
| Gallus gallus | MNQKHLLRFIKKSYRVDADRVVYDAK | SEQ ID |
| AMPD1 | | NO. 217 |
| Mus musculus | MPLFKLTGQGKQIDDAMRSFAEKVFASEVKDEGGRHEISP | SEQ ID |
| AMPD1 | FDVDEICPISLHEMQAHIFHMENLSMDGRRKRRFQGRKTV | NO. 218 |
| | NLSIPQSETSSTKLSHIEEFISSSPTYESVPDFQRVQITGDYAS | |
| | GVTVEDFEVVCKGLYRALCIREKYMQKSFQRFPKTPSKYL | |
| | RNIDGEALVGNESFYPVFTPPPKKGEDPFRTEDLPANLGYH | |
| | LKMKAGVIYIYPDEAAANRDEPKPYPYPNLDDFLDDMNFL | |
| | LALIAQGPVKTYAHRRLKFLSSKFQVHQMLNEMDELKEL | |
| | KNNPHRDFYNCRKVDTHIHAAACMNQKHLLRFIKKSYHI | |
| | DADRVVYSTKEKSLTLKELFAKLNMHPYDLTVDSLDVHA | |
| | GRQTFQRFDKFNDKYNPVGASELRDLYLKTDNYINGEYFA | |
| | TIIKEVGADLVEAKYQHAEPRLSIYGRSPDEWNKLSSWFV | |
| | CNRIYCPNMTWMIQVPRIYDVFRSKNFLPHFGKMLENIFLP | |
| | VFEATINPQAHPDLSVFLKHITGFDSVDDESKHSGHMFSSK | |
| | SPKPEEWTMENNPSYTYYAYYMYANITVLNSLRKERGMN | |
| | TFLFRPHCGEAGALTHLMTAFMIADNISHGLNLKKSPVLQ | |
| | YLFFLAQIPIAMSPLSNNSLFLEYAKNPFLDFLQKGLMISLS | |
| | TDDPMQFHFTKEPLMEEYAIAAQVFKLSTCDMCEVARNS | |
| | VLQCGISHEEKAKFLGNNYLEEGPVGNDIRRTNVAQIRMA | |
| | YRYETWCYELNLIAEGLKATE | |
| Dictyostelium | MSTPLRGSSPQVSFYESELDQEGGSDASHFTYRNYMEDDK | SEQ ID |
| discoideum AMPD1 | INSFTFNMARKDQTQLFQRIILTNESESEIEEYAEVAEQLLD | NO. 219 |
| | AINLREKYVFHPKIWKADAPVGEKPPYSPFESDESTNCATE | |
| | HMFKEVNGVYFVYSNETDMKSNKALFSVPHTLASYYKDI | |
| | NNLMMLSSYGPAKTFTFKRLQLLESKFNMHTLLNDSLELF | |
| | QQKTAPHRDFYNVRKVDTHVHHSSSMNQKHLLKFIKRKL | |
| | KENPNEIVIFRDDKYLTLAEVFKSLNLDVDELSVDTLDVH | |
| | ADNNTFHRFDKFNLKYNPCGQSRLREIFLKTDNLIKGKYL | |
| | AEISKEVFTDLESSKYQCAEYRLSIYGRKMSEWDTLASWI | |
| | VDNDLFSTKVRWLIQVPRLYDVYRETSTTTFQDFLNNVFH | |
| | PLFEVTKDPSSHPKLHLFLQQVVGIDCVDDESKFEKKFTEK | |
| | FPVPGEWSSEHNPPYTYYLYYLYANLYTLNQFREEKGLNI | |
| | LTLRPHSGEAGEVDHMGAAFYLAHGINHGINLRKTPVLQY | |
| | LYYLTQIGIAMSPLSNNSLFLTYNRNPFPAFFARGLNVSIST | |
| | DDPLQFHYTKEPLMEEYSIATQVWRLSVCDICEIARNSVLQ | |
| | SGFEHNVKSHWLGPDYANSGGNDIKKTNISDIRVCFRNET | |
| | LIEELHLILKSLQTLPNFKNLNINFLLDKLPSEITTGNDYKLK | |
| | KAQLKLNGANKLRNSSVGSTPNNGTPSSSGTPSLSSPGAIV | |
| | HLMKTKPYIPPPLSLNIKQENNNNNNNNNNNNNNNNNNN | |
| | TNTNTNSNSTTTNQDDNSKSDK | |
| Human AMPD3 | MPRQFPKLNISEVDEQVRLLAEKVFAKVLREEDSKDALSL | SEQ ID |
| | FTVPEDCPIGQKEAKERELQKELAEQKSVETAKRKKSFKM | NO. 220 |
| | IRSQSLSLQMPPQQDWKGPPAASPAMSPTTPVVTGATSLPT | |
| | PAPYAMPEFQRVTISGDYCAGITLEDYEQAAKSLAKALMI | |
| | REKYARLAYHRFPRITSQYLGHPRADTAPPEEGLPDFHPPP | |
| | LPQEDPYCLDDAPPNLDYLVHMQGGILFVYDNKKMLEHQ | |
| | EPHSLPYPDLETYTVDMSHILALITDGPTKTYCHRRLNFLE | |
| | SKFSLHEMLNEMSEFKELKSNPHRDFYNVRKVDTHIHAAA | |
| | CMNQKHLLRFIKHTYQTEPDRTVAEKRGRKITLRQVFDGL | |
| | HMDPYDLTVDSLDVHAGRQTFHRFDKFNSKYNPVGASEL | |
| | RDLYLKTENYLGGEYFARMVKEVARELEESKYQYSEPRLS | |
| | IYGRSPEEWPNLAYWFIQHKVYSPNMRWIIQVPRIYDIFRS | |
| | KKLLPNFGKMLENIFLPLFKATINPQDHRELHLFLKYVTGF | |
| | DSVDDESKHSDHMFSDKSPNPDVWTSEQNPPYSYYLYYM | |
| | YANIMVLNNLRRERGLSTFLFRPHCGEAGSITHLVSAFLTA | |
| | DNISHGLLLKKSPVLQYLYYLAQIPIAMSPLSNNSLFLEYSK | |
| | NPLREFLHKGLHVSLSTDDPMQFHYTKEALMEEYAIAAQV | |
| | WKLSTCDLCEIARNSVLQSGLSHQEKQKFLGQNYYKEGPE | |
| | GNDIRKTNVAQIRMAFRYETLCNELSFLSDAMKSEEITALT | |
| | N | |
| Human AMPD2 | MRNRGQGLFRLRSRCFLHQSLPLGAGRRKGLDVAEPGPSR | SEQ ID |
| | CRSDSPAVAAVVPAMASYPSGSGKPKAKYPFKKRASLQA | NO. 221 |
| | STAAPEARGGLGAPPLQSARSLPGPAPCLKHFPLDLRTSMD | |
| | GKCKEIAEELFTRSLAESELRSAPYEFPEESPIEQLEERRQRL | |
| | ERQISQDVKLEPDILLRAKQDFLKTDSDSDLQLYKEQGEG | |
| | QGDRSLRERDVLEREFQRVTISGEEKCGVPFTDLLDAAKS | |
| | VVRALFIREKYMALSLQSFCPTTRRYLQQLAEKPLETRTYE | |
| | QGPDTPVSADAPVHPPALEQHPYEHCEPSTMPGDLGLGLR | |
| | MVRGVVHVYTRREPDEHCSEVELPYPDLQEFVADVNVLM | |
| | ALIINGPIKSFCYRRLQYLSSKFQMHVLLNEMKELAAQKK | |
| | VPHRDFYNIRKVDTHIHASSCMNQKHLLRFIKRAMKRHLE | |
| | EIVHVEQGREQTLREVFESMNLTAYDLSVDTLDVHADRNT | |
| | FHRFDKFNAKYNPIGESVLREIFIKTDNRVSGKYFAHIIKEV | |
| | MSDLEESKYQNAELRLSIYGRSRDEWDKLARWAVMHRV | |
| | HSPNVRWLVQVPRLFDVYRTKGQLANFQEMLENIFLPLFE | |
| | ATVHPASHPELHLFLEHVDGFDSVDDESKPENHVFNLESPL | |
| | PEAWVEEDNPPYAYYLYYTFANMAMLNHLRRQRGFHTF | |
| | VLRPHCGEAGPIHHLVSAFMLAENISHGLLLRKAPVLQYL | |
| | YYLAQIGIAMSPLSNNSLFLSYHRNPLPEYLSRGLMVSLST | |
| | DDPLQFHFTKEPLMEEYSIATQVWKLSSCDMCELARNSVL | |
| | MSGFSHKVKSHWLGPNYTKEGPEGNDIRRTNVPDIRVGYR | |
| | YETLCQELALITQAVQSEMLETIPEEAGITMSPGPQ | |
| Mus musculus | MASEARSGLGASPLQSARSLPGNAPCLKHFPLDLRTSMDG | SEQ ID |
| AMPD2 | KCKEIAEELFSRSLAESELRSAPYEFPEESPIEQLEERRQRLE | NO. 222 |
| | RQISQDVKLEPDILLRAKQDFLKTDSDSDLQLYKEQGEGQ | |
| | GDRGLWERDVVLEREFQRVIISGEEKCGVPFTDLLDAAKS | |
| | VVRALFIREKYMALSLQSFCPTTRRYLQQLAEKPLETRTYE | |
| | QSPDTPVSADAPVHPPALEQHPYEHCEPSAMPGDLGLGLR | |
| | MVRGVVHVYTRRDPDEHCPEVELPYPDLQEFVADVNVLM | |
| | ALIINGPIKSFCYRRLQYLSSKFQMHVLLNEMKELAAQKK | |
| | VPHRDFYNIRKVDTHIHASSCMNQKHLLRFIKRAMKRHLE | |
| | EIVHVEQGREQTLREVFESMNLTAYDLSVDTLDVHADRNT | |
| | FHRFDKFNAKYNPIGESVLREIFIKTDNKISGKYFAHIIKEV | |
| | MADLEESKYQNAELRLSIYGRSRDEWDKLARWAVNHKV | |
| | HSPNVRWLVQVPRLFDVYRTKGQLANFQEMLENIFLPLFE | |
| | ATVHPASHPELHLFLEHVDGFDSVDDESKPENHVFNLESPL | |
| | PEAWVEEDNPPYAYYLYYTFANMAMLNHLRRQRGFHTF | |
| | VLRPHCGEAGPIHHLVSAFMLAENISHGLLLRKAPVLQYL | |
| | YYLAQIGIAMSPLSNNSLFLSYHRNPLPEYLSRGLMVSLST | |
| | DDPLQFHFTKEPLMEEYSIATQVWKLSSCDMCELARNSVL | |
| | MSGFSHKVKSHWLGPNYTKEGPEGNDIRRTNVPDIRVGYR | |
| | YETLCQELALITQAVQSEMLETIPEEVGIVMSPGP | |
| Rattus norvegicus | MASYPGPGKSKAKYPFKKRASLQASAAAPEARSGLGASPL | SEQ ID |
| AMPD2 | QSARSLPGTAPCLKHFPLDLRTSMDGKCKEIAEELFSRSLA | NO. 223 |
| | ESELRSAPYEFPEESPIEQLEERRQRLERQISQDVKLEPDILL | |
| | RAKQDFLKTDSDSDLQLYKEQGEGQGDRGLWERDVVLER | |
| | EFQRVIISGEEKCGVPFTDLLDAAKSVVRALFIREKYMALS | |
| | LQSFCPTTRRYLQQLAEKPLETRTYEQSPDTPVSADAPVHP | |
| | PALEQHPYEHCEPSTMPGDLGLGLRMVRGVVHVYTRRDP | |
| | DEHCPEVELPYPDLQEFVADVNVLMALIINGPIKSFCYRRL | |
| | QYLSSKFQMHVLLNEMKELAAQKKVPHRDFYNIRKVDTH | |
| | IHASSCMNQKHLLRFIKRAMKRHLEEIVHVEQGREQTLRE | |
| | VFESMNLTAYDLSVDTLDVHADRNTFHRFDKFNAKYNPI | |
| | GESVLREIFIKTDNKISGKYFAHIIKEVMSDLEESKYQNAEL | |
| | RLSIYGRSRDEWDKLARWAVNHRVHSPNVRWLVQVPRLF | |
| | DVYRTKGQLANFQEMLENIFLPLFEATVHPASHPELHLFLE | |
| | HVDGFDSVDDESKPENHVFNLESPLPEAWVEEDNPPYAYY | |
| | LYYTFANMAMLNHLRRQRGFHTFVLRPHCGEAGPIHHLV | |
| | SAFMLAENISHGLLLRKAPVLQYLYYLAQIGIAMSPLSNNS | |
| | LFLSYHRNPLPEYLSRGLMVSLSTDDPLQFHFTKEPLMEEY | |
| | SIATQVWKLSSCDMCELARNSVLMSGFSHKVKSHWLGPN | |
| | YTKEGPEGNDIRRTNVPDIRVGYRYETLCQELALITQAVQS | |
| | EMLETIPEEVGIVMSPGP | |
| Plasmodium | MTILHEEINFLKKDELNINLKCLDKKERYKIWRRIPKCELH | SEQ ID |
| gallinaceum ADA | CHLDLCFSLEFFLKCVRKYNLQPDLTDDEVVDYYLFKDKG | NO. 224 |
| | KSLNEFIERSRRVTDIFINYDIIKDIAKNAVFNKYKEGVILIE | |
| | FRYSPSYIAYKYNLCIDLIHKTIVEGINEAVEKLNHKIHVGL | |
| | ICIGETGISEESLRKAAEFCVKNKKDFVGFDHAGHERDLKP | |
| | YKEIYDYVRENGIPLTIHAGEDLTLPNLNTIYSAIEVLKAKR | |
| | IGHGIRVIESEDLINLIKKNDILLEICPISNLLLNNVKSMDTH | |
| | PIKKLYDSGIKVSVNTDDPGMFLTEINDEYEELYLNLNFNL | |
| | EDFMKMNLWALEKSFVKSEIKDKLKKLYF | |
| Plasmodium | MNILQEPIDFLKKDELKNIDLSQMDKKERYKIWKRIPKCEL | SEQ ID |
| knowlesi ADA | HCHLDLCFSADFFLSCVRKYNLQPNLSDEEVLDYYLFAKG | NO. 225 |
| | GKSLGEFVEKAIRVADIFQDYEMIEDLAKHAVFNKYKEGV | |
| | VLMEFRYSPTFVAFKHNLDIELIHQAIVKGIKEVVELLDHKI | |
| | DVTLLCIGDTGHRAADIKASADFCLKHKADFVGFDHGGH | |
| | EVDLKPYKEIFDYVKEGGMHLTVHAGEDVTLPNLNTLYS | |
| | AIQVLKVERIGHGIRVSESQELIDMVKENNILLEVCPISNVL | |
| | LKNAKSFDTHPIRKLYDAGVKVSVSSDDPGMFLTNINDDY | |
| | EKLYTHLHFTLEDFMKMNEWALEKSFIGCDIKEKIKKLYF | |
| Plasmodium berghei | MEIPNEEIKFLKKEDIKNINLNGMNKKERYEIWKKIPKVEL | SEQ ID |
| ADA | HCHLDLTFSGKFFLKWVRKYNLQPNMTDDQVLDHYLFTK | NO. 226 |
| | EGKSLAEFIRKAISVSDIYRDYDILEDLAKWAVIEKYKEGV | |
| | VLMEFRYSPTFVSSSHGLDIELIHKAFVKGIKNATEMLNNK | |
| | IYVALICISDTGHSAASIKHSGDFAIKHKHDFVGFDHGGREI | |
| | DLKDHKDVYHSVRNHGLHLTVHAGEDATLPNLNTLYTAI | |
| | NILNVERIGHGIRVSESEELIELVKKNNILLEVCPISNLLLNN | |
| | VKSMDTHPIRKLFDAGVKVSVNSDDPGMFLTDINDNYEKL | |
| | YIHLNFTLEEFMTMNNWALEKSFVNDDIKSKLKTMYF | |
| Plasmodium vivax | MNILQEPIDFLKKEELKNIDLSQMSKKERYKIWKRIPKCEL | SEQ ID |
| ADA | HCHLDLCFSADFFVSCIRKYNLQPNLSDEEVLDYYLFAKG | NO. 227 |
| | GKSLGEFVEKAIKVADIFHDYEVIEDLAKHAVFNKYKEGV | |
| | VLMEFRYSPTFVAFKYNLDIELIHQAIVKGIKEVVELLDHKI | |
| | HVALMCIGDTGHEAANIKASADFCLKHKADFVGFDHGGH | |
| | EVDLKEYKEIFDYVRESGVPLSVHAGEDVTLPNLNTLYSAI | |
| | QVLKVERIGHGIRVAESQELIDMVKEKNILLEVCPISNVLL | |
| | KNAKSMDTHPIRQLYDAGVKVSVNSDDPGMFLTNINDDY | |
| | EELYTHLNFTLEDFMKMNEWALEKSFMDSNIKDKIKNLYF | |
| Plasmodium | MNILQEPIDFLKKDEIKNIDLSQMSKKERYKIWKRIPKCEL | SEQ ID |
| cynomolgi ADA | HCHLDLCFSADFFLSCIRKYNLQPNLSDEEVLDYYLFAKG | NO. 228 |
| | GKSLGEFVEKAIRVADIFHDYEVIEDLAKHAVFNKYKEGV | |
| | VLMEFRYSPTFVAFKYKLDIELIHQAIVKGIKEVVELLDHKI | |
| | HVALMCIGDTGHEAANIKASADFCLKHRADFVGFDHGGH | |
| | EVDLKQYKEIFDYVRESGIPLSVHAGEDVTLPNLNTLYSAI | |
| | QVLKVERIGHGIRVSESQELIDMVKEKNILLEVCPISNVLLK | |
| | NAKSMDTHPIRQLYDAGVKVSVNSDDPGMFLTNINDDYE | |
| | ELYTHLNFTLEDFMKMNEWALEKSFMDSNIKDKVKNLYF | |
| Human ADA | MAQTPAFDKPKVELHVHLDGSIKPETILYYGRRRGIALPAN | SEQ ID |
| | TAEGLLNVIGMDKPLTLPDFLAKFDYYMPAIAGCREAIKRI | NO. 229 |
| | AYEFVEMKAKEGVVYVEVRYSPHLLANSKVEPIPWNQAE | |
| | GDLTPDEVVALVGQGLQEGERDFGVKARSILCCMRHQPN | |
| | WSPKVVELCKKYQQQTVVAIDLAGDETIPGSSLLPGHVQA | |
| | YQEAVKSGIHRTVHAGEVGSAEVVKEAVDILKTERLGHG | |
| | YHTLEDQALYNRLRQENMHFEICPWSSYLTGAWKPDTEH | |
| | AVIRLKNDQANYSLNTDDPLIFKSTLDTDYQMTKRDMGFT | |
| | EEEFKRLNINAAKSSFLPEDEKRELLDLLYKAYGMPPSASA | |
| | GQNL | |
| Mus musculus ADA | MAQTPAFNKPKVELHVHLDGAIKPETILYFGKKRGIALPA | SEQ ID |
| | DTVEELRNIIGMDKPLSLPGFLAKFDYYMPVIAGCREAIKR | NO. 230 |
| | IAYEFVEMKAKEGVVYVEVRYSPHLLANSKVDPMPWNQT | |
| | EGDVTPDDVVDLVNQGLQEGEQAFGIKVRSILCCMRHQPS | |
| | WSLEVLELCKKYNQKTVVAMDLAGDETIEGSSLFPGHVE | |
| | AYEGAVKNGIHRTVHAGEVGSPEVVREAVDILKTERVGH | |
| | GYHTIEDEALYNRLLKENMHFEVCPWSSYLTGAWDPKTT | |
| | HAVVRFKNDKANYSLNTDDPLIFKSTLDTDYQMTKKDMG | |
| | FTEEEFKRLNINAAKSSFLPEEEKKELLERLYREYQ | |
| Bos taurus ADA | MAQTPAFNKPKVELHVHLDGAIKPETILYYGRKRGIALPA | SEQ ID |
| | DTPEELQNIIGMDKPLSLPEFLAKFDYYMPAIAGCREAVKR | NO. 231 |
| | IAYEFVEMKAKDGVVYVEVRYSPHLLANSKVEPIPWNQA | |
| | EGDLTPDEVVSLVNQGLQEGERDFGVKVRSILCCMRHQPS | |
| | WSSEVVELCKKYREQTVVAIDLAGDETIEGSSLFPGHVKA | |
| | YAEAVKSGVHRTVHAGEVGSANVVKEAVDTLKTERLGH | |
| | GYHTLEDATLYNRLRQENMHFEVCPWSSYLTGAWKPDTE | |
| | HPVVRFKNDQVNYSLNTDDPLIFKSTLDTDYQMTKNEMG | |
| | FTEEEFKRLNINAAKSSFLPEDEKKELLDLLYKAYGMPSPA | |
| | SAEQCL | |
| Gallus gallus ADA | MERGVRVFGEPKVELHIHLDGAIRPETILHFGKKRGVPLPG | SEQ ID |
| | STVDELMKHVSYQTPLSLKLFLEKFNHYMPAIAGDREAVR | NO. 232 |
| | RIAYELVETKAKEGVVYVEVRYSPHLLANCRVEPIPWGQA | |
| | EGDLTPEEVVNLVNQGLQDGERNFRIKARSILCCMRHMPS | |
| | WSPEVVELCKKYQNNSVVAIDLAGDELLMASSDHKAAYE | |
| | EAERCGIHRTVHAGEAGPATMIKEAVYLLKAERIGHGYHV | |
| | LEDPELYRELLRTRMHFEVCPWSSYLTGACLPDFRKHPVV | |
| | QFKKDQANYSINTDDPLIFNSNIDKDYGIVKEYMDFTEEDF | |
| | KRVNINAAQSSFLPEKEKQELLNTLYEAYGMVPATS | |
| Danio rerio ADA | MNGKPAFDKPKVELHVHLDGAIRLKTVLDVAKRRGISLPV | SEQ ID |
| | SMEEELKELCTVNEPATLTEFLGKFSHFMHVIAGDREAIKR | NO. 233 |
| | IAYEFVETKAKEGVIYVEARYSPHFLANKGVEPLPWDQKP | |
| | GDITPDDVVDLVNQGFKEGEQAFKTKARSILCCMRHMPN | |
| | WSMEVVELCKKFHKDGVVAIDLAGDESMNCESYPGHKK | |
| | AFEEAVRSNVHRTVHAGEVGPASVVREAVEVLKAERIGH | |
| | GYHTLEDQNLYKQLLHQNMHFEMCPVSSRLTGACEPDFT | |
| | KHPLITFKKDKANYSLNTDDPTIFNSTLNSDYEVVQKYMD | |
| | FTEEEFKRLNINAAKSCFLPEKEKEKLLNQLYEAYGMRKS | |
| | TSF | |
| Xenopus laevis ADA | MESKAFNKPKVELHVHLDGSIKPETIIHFAKKRQIKLPADT | SEQ ID |
| | VEGLLEHVSYKEPLSLTEFLQKFNHYMPAIAGDREAIKRIA | NO. 234 |
| | YEFVEMKAKEGVIYVEVRYSPHFLANSKVDPIPWGQKEG | |
| | DITPDEVVDLVNQGLRKGEKTFNIKARSILCCMRHMPNWS | |
| | SEVIELCKKYQNDTVVAIDLAGDESLNCESYPGHRKAYEE | |
| | AVKCGIHRTVHAGEVGPPSVVKEAVEVLKAERIGHGYHTT | |
| | EDPNLYKELLENNMHFEVCPWSSYLTSACHPDFTKHPATQ | |
| | FRKDKANFSLNTDDPLIFGSTLDVDYSIAVQHMGFTEDEFK | |
| | RVNINAAKSSFLPDNEKKELLYKLYEAYGMILSTGL | |
| Plasmodium | MNCKNMDTSYEIINYLTKDELDIDLSCMDKKERYKIWKRL | SEQ ID |
| falciparum ADA | PKCELHCHLDVCFSVDFFLNVIRKYNIQPNMSDEEIIDYYL | NO. 235 |
| | FSKPGKSLDEFVEKALRLTDIYIDYTVVEDLAKHAVENKY | |
| | KEGVVLMEFRYSPSFMSFKHNLDKDLIHEAIVKGLNEAVA | |
| | LLEYKIQVGLLCTGDGGLSHERMKEAAEFCIKHKKDFVGY | |
| | DHAGHEVDLKPFKDIFDNIREEGISLSVHAGEDVSIPNLNSL | |
| | YTAINLLHVKRIGHGIRVSESQELIDLVKEKDILLEVCPISN | |
| | VLLNNVKSMDTHPIRMLYDAGVKVSVNSDDPGMFLTNIT | |
| | DNYEELYTHLNFTLADFMKMNLWAVQKSFVDPDIKNKIIS | |
| | KYF | |
| Drosophila | MEQFLKGLPKVELHAHLNGSLGIKSLCDLGERLYGTSCKD | SEQ ID |
| melanogaster | FLKLCAHFSRFEKDMDACFEKFAFVHELTSTREGLRFATEL | NO. 236 |
| | AIRDFAEDNVQYVEMRTTPKANENYSRRDYLQIVIDAIKA | |
| | ASETYPEITVKLLPSINRAEPVDVAEETVSLAVELARAHPN | |
| | LILGIDLSGNPGKGRFSDFAPILAQARDKGLKLAIHCAEIEN | |
| | PSEVKEMLHFGMSRCGHGTFLTPEDIGQLKQRNIAIECCLT | |
| | SNVKSGTVPSLEEHHLKRIMEADAPKVICTDDSGVFDTTLT | |
| | KEFLIAAETFGLTREQCIDLTLEAVHHSFASEQEQIQMADR | |
| | VGNYADILVK | |
| Xenopus tropicalis | MESKAFNKPKVELHVHLDGSIKPETIIHFAKKRQIKLPADT | SEQ ID |
| ADA | VEGLLEHVSYKEPLSLTEFLSKFNHYMPAIAGDREAIKRIA | NO. 237 |
| | YEFVEMKAKEGVIYVEVRYSPHFLANSKVEPIPWGQKEGD | |
| | ITPDEVVDLVNQGLRKGEKAFNIKARSILCCMRHMPSWST | |
| | EVVELCKKYQNDTVVAIDLAGDESLNCESYPGHRKAYEE | |
| | AVKCGIHRTVHAGEVGPSSVVKEAVEVLKAERIGHGYHTT | |
| | EDPNLYKELLEKNMHFEVCPWSSYLTGACHPDFTKHPAT | |
| | QFRKDKANYSLNTDDPLIFGSTLDVDYSIAAKHMGFTEEE | |
| | FKRVNINAAKSSFLPESEKKELLYKLYEAYGMILSTGL | |
| Rattus norvegicus | MAQTPAFNKPKVELHVHLDGAIKPETILYYGKKRGIDLPA | SEQ ID |
| ADA | DTVEGLRNIIGMDKPLSLPDFLAKFDYYMPAIAGCREAIKR | NO. 238 |
| | IAYEFVEMKAKEGVVYVEVRYSPHLLANSKVDPIPWNQA | |
| | EGDLTPDEVVDLVNQGLQEGEQAFGIKVRSILCCMRHQPS | |
| | WSPEVLELCKKYHQKTVVAMDLAGDETIEGSSLFPGHVE | |
| | AYEGAVKDGIHRTVHAGEVGSAEVVREAVDILKTERVGH | |
| | GYHTIEDEALYNRLLKENMHFEVCPWSSYLTGAWNPKTT | |
| | HAVVRFKDDQANYSLNSDDPLIFKSTVDTDYQMVKKDM | |
| | GFTEEEFKRLNINAAKSSFLPEDEKKELLERLYKEYQ | |
| Mus musculus | MATAGGSRRAPVPGPRLGLPLAAHLPASLGGEGAKDSVG | SEQ ID |
| ADAD1 | GEKTSGNNDWFQSSRVPSFAQMLKKNLPVQPSAQTVTLPT | NO. 239 |
| | GYSSESCSLSNMASKVTQVTGNFPEPLLSKGLSSISNPVLPP | |
| | KKLPKEFIMKYKRGEINPVSALHQFAQMQRVQLDLKETVT | |
| | TGNVMGPYFAFCAVVDGIQYKTGLGQNKKESRSNAAKLA | |
| | LDELLQLDEPEPRVLEPAGPPPIPAEPVVTPEAAYVSKVQY | |
| | EGRQVQYAKISQLVKETFGQLISNHSQYLKCSSSLAAFIIER | |
| | AGHHEVVAIGTGEYNYSQCIKPNGRVLHDTHAVVTARRS | |
| | LLRYFYRQLLLFYSKNPAMMEKSIFCTEPASNLLTLKQNIN | |
| | LYLYMNQLPKGSAQIKSQLRLNPHSISAFEANEELSLHVAV | |
| | EGKIYLTVYCSADGVNRVNSMSSSDKLTRWEVLGVQGAL | |
| | LSHFIQPVYISSILVGDGNCNDTRGLEIAINQRVDDALTSKL | |
| | PMFYLVNRPHISLVPTAYPLQINLDHKSLSLNWAQGDNSL | |
| | EIVDGLNGKITESSPFKSGLSMASRLCKAAMLSRFNLLAKE | |
| | AKTDDLLEARTYHAAKCMSGPYQEAKALLKAYLQQHGY | |
| | GSWIVKSPCIEQFSM | |
| Rattus norvegicus | MATAGGSRRAPVPGPRLGLPLAAHLPASLGGEGAKDSLG | SEQ ID |
| ADAD1 | GEKTSGNNDWFQSSRVPSFAQMLKKNLPVQSSAQTVTLPT | NO. 240 |
| | GYSSESCSLSNMASKVTQVTGNFPEPLLSKGLSSISNPVLPP | |
| | KKIPKEFIMKYRRGEINPVSALHQFAQMQRVQLDLKETVT | |
| | TGNVMGPYFAFCAVVDGIQYKTGLGQNKKESRSNAAKLA | |
| | LDELLQLDEPEPRALEPAGPPPIPAEPIVTPEAAYISKVQYE | |
| | GRQVQYAKISQLVKETFSQLISSHSQYLKCSSSLAAFIIERG | |
| | GHHEVVAIGTGEYNYSQCIKPNGRVLHDTHAVVTARRSLL | |
| | RYFYRQLLLFYSKNPAMMEKSIFCTEPASNLLTLKQNINLY | |
| | LYMNQLPKGSAQIKSQLRLNPHSISAFEANEELSLHVAVEG | |
| | KIYLTVYCSADGVNRVNSMSSSDKLTRWEVLGVQGALLS | |
| | HFIQPVYISSILVGDGNCNDTRGLEIAINQRVDDALTSKLP | |
| | MFYLVNRPHISLVPTAYPLQINLDHKSLSLNWAQGDNSLEI | |
| | VDGLSGKITESSPFKSGLSMASRLCKAAMLSRFNLLAKEA | |
| | KTDDLLEARTYHAAKCLSGPYQEAKALLKAYLQQHGYGS | |
| | WIVKSPCIEQFSM | |
| Xenopus laevis | MASNRNWSQHSSVPSFAQMLKKNLPDPGTSPAVNQTSTLS | SEQ ID |
| ADAD1 | TCCLYNQPDCGTARVTRITGNFPEPFLSKMIVPPSLSSLTPR | NO. 241 |
| | KVTKEFMVKYRRGDLNPISALYQFAQMQRMEIELKETVTT | |
| | GNVFGAYFAFCAVVDGLEYKTGMGQNKKEAKANAAKLA | |
| | LDELLLHEDPALIDSENQSLNVIENPPPLPMNPRGTTETSTIS | |
| | RTRTDKRTFIHEKISSIIKETFTNLVSKYPEYENCGSSLAAFV | |
| | IEKGGQHWEVVAIGTGEFNYGQSLQSDGRVLHDSHAMVV | |
| | ARRSLLRYFYRQLLLLYSGNNGMMDKSIFCTEPATNLLAL | |
| | KPNLNIFLYMNQLPKGAAQTNPQLCLSPHSLSAHEANDKL | |
| | SLHVSVEGKNIPASYYSGEIVHNLYSMSSTDKLTRWEVLG | |
| | VQGALLSIFIQPVYINSIIIGNAACSDTRGLEIAVKQRIDDAL | |
| | TSRLPMFYLVNRPYMSIVSSTHLTNNDTANKTLSLNWSQG | |
| | DACVEVVDAAIGRTVEGSPFKSGSCLASRLCKAAMLCRFN | |
| | LVVKESKRNAIPSGLSYHEAKRLAGPYQEAKCLLNSYFKQ | |
| | QGFGSWIAKPPIIGEFTM | |
| Human ADAD1 | MASNNHWFQSSQVPSFAQMLKKNLPVQPATKTITTPTGW | SEQ ID |
| | SSESYGLSKMASKVTQVTGNFPEPLLSKNLSSISNPVLPPK | NO. 242 |
| | KIPKEFIMKYKRGEINPVSALHQFAQMQRVQLDLKETVTT | |
| | GNVMGPYFAFCAVVDGIQYKTGLGQNKKESRSNAAKLAL | |
| | DELLQLDEPEPRILETSGPPPFPAEPVVLSELAYVSKVHYEG | |
| | RHIQYAKISQIVKERFNQLISNRSEYLKYSSSLAAFIIERAGQ | |
| | HEVVAIGTGEYNYSQDIKPDGRVLHDTHAVVTARRSLLRY | |
| | FYRQLLLFYSKNPAMMEKSIFCTEPTSNLLTLKQNINICLY | |
| | MNQLPKGSAQIKSQLRLNPHSISAFEANEELCLHVAVEGKI | |
| | YLTVYCPKDGVNRISSMSSSDKLTRWEVLGVQGALLSHFI | |
| | QPVYISSILIGDGNCSDTRGLEIAIKQRVDDALTSKLPMFYL | |
| | VNRPHISLVPSAYPLQMNLEYKFLSLNWAQGDVSLEIVDG | |
| | LSGKITESSPFKSGMSMASRLCKAAMLSRFNLLAKEAKKE | |
| | LLEAGTYHAAKCMSASYQEAKCKLKSYLQQHGYGSWIV | |
| | KSPCIEQFNM | |
| Bos taurus ADAL | MMEAEEQPWKTTFYSKLPKVELHAHLNGSISSNTIRKLIAK | SEQ ID |
| | KPDLKIHDQMTMIDKGEKRTLEECLQMFQIIHLLTTTPEDV | NO. 243 |
| | LMVTKDVIKEFADDGVKYLELRSTPRGEDATGMTKKTYV | |
| | ESILEGIKQSKEENVDIDVRYLISIDRRGGSSAAKEAVKLAE | |
| | EFFLSAEDTVLGLDLSGDPSAGQAKDFLEPLLEAKKSGLKL | |
| | ALHLSEIPNQKTETQVLLNLFPDRIGHGTFLSSSEEGSPDLV | |
| | DFVRQHQIPLELCLTSNVKSQTVPAYDQHHFGFWYSVAHP | |
| | AVICTDDKGVFATRLSQEYQLVAETFHLTQSQVWDLSYES | |
| | ISYIFASDSTKADLRKKWSHLKPHF | |
| Danio rerio ADAL | MDTEADLFYRQLPKVELHAHLNGSVSFETMEKLIKRKPHL | SEQ ID |
| | NIEHSMTAIRRGQRRTLDECFQVFKVIHQLVDSEEDILMVA | NO. 244 |
| | KSVIQEFAADGVKYLELRSTPREVTETGLSKQRYIETVLEA | |
| | IRQCKQEGVDIDVRFLVAVDRRHGPEVAMQTVKLAEDFL | |
| | LSSDGTVVGLDLSGDPTVGHGKDLLAALQKAKNCGLKLA | |
| | LHLSEVPSQIDETELLLNLPPDRIGHGTFLHPDVGGSDSLVD | |
| | KVCKQNIPIEICLTSNVKGQTVPSYDKHHFKYWYNRGHPC | |
| | VLCTDDKGVFCTDLSQEYQLAASTFGLTKEAVWILSQQAI | |
| | GYTFAPEPIKQRLEKTWAELKQQILQ | |
| Xenopus laevis | MAGEGALQFYRDLPKVELHAHLNGSISTATMKKLMARKP | SEQ ID |
| ADAL | HLDIQHGMTMIDKGQKRTLEECFQMFKIIHQITDTAEDILL | NO. 245 |
| | VTKDVIKEFAADGVKYLELRSTPRDTPAGLTKQAYVETVL | |
| | EGIKQCKEEGVDIDVRFLLAIDRRGGPTAAKETVKLAEDFF | |
| | CSSNELVLGLDLSGDPTVGHGRDFMEPLNKARQSGLKLAL | |
| | HLSEIPSQTEETELLLGLPPDRIGHGTFLTTSAHIVEIVKKQH | |
| | IPLELCITSNIKGQTVSSYNEHHFGFWYNLHHPFVLCTDDK | |
| | GVFATDLSVEYEIAAKTFNLTPHHVWDLSYQAIDYTFASA | |
| | DVKANLKEKWLLLKPDVFRHAL | |
| Human ADAL | MIEAEEQQPCKTDFYSELPKVELHAHLNGSISSHTMKKLIA | SEQ ID |
| | QKPDLKIHDQMTVIDKGKKRTLEECFQMFQTIHQLTSSPED | NO. 246 |
| | ILMVTKDVIKEFADDGVKYLELRSTPRRENATGMTKKTYV | |
| | ESILEGIKQSKQENLDIDVRYLIAVDRRGGPLVAKETVKLA | |
| | EEFFLSTEGTVLGLDLSGDPTVGQAKDFLEPLLEAKKAGL | |
| | KLALHLSEIPNQKKETQILLDLLPDRIGHGTFLNSGEGGSLD | |
| | LVDFVRQHRIPLELCLTSNVKSQTVPSYDQHHFGFWYSIA | |
| | HPSVICTDDKGVFATHLSQEYQLAAETFNLTQSQVWDLSY | |
| | ESINYIFASDSTRSELRKKWNHLKPRVLHI | |
| Caenorhabditis | MPNNSKHKKKQQRRQQEAQKKSRAKQIETDKKNDEFLDT | SEQ ID |
| elegans ADAL | ELDEVSPLVIDDDMTEFKNMPKVELHAHLSGSLSPETIKLI | NO. 247 |
| | MESDETRAEEIMKKYKLEKPENMTGVFDCFPVIHAILRKPE | |
| | AIRIAIRQTIKEFEEDNCVYLELRTSPKETDFMTYEDYLQVC | |
| | IESFEAAKHEFPRIKTFLIVSLDRRMPFETAAHILGLIGEAQQ | |
| | RTNVIVGVELSGDPHLDGRRLLKLFVAARRFHGLGITIHLA | |
| | EVLQNMADVEDYLNLRPDRIGHGTFLHTDPYTEYLTNKY | |
| | KIPLEICLSSNVYSKTTTNYRNSHFNYWRKRGVPVFICTDD | |
| | KGVIPGATLTEEYYKAAITFDLSTEELIGINQDALLNSFAYK | |
| | YNVTDLTETFRKINNNVLD | |
| Mus musculus | MEAGQQWPGKTDFYLQLPKVELHAHLNGSISSSTMKKLIA | SEQ ID |
| ADAL | KKPHLNVHGHMTMIDKGKKRTLQECFQMFQVIHQLTTSA | NO. 248 |
| | EDILMVTKDVIKEFADDGVKYLELRSTPREENATGMTRKT | |
| | YVESVLEGIKQCKQENLDIDVRYLMAIDRRGGPTIARETVE | |
| | LAKEFFLSTENTVLGLDLSGDPTIGQANDFLEPLLEAKKAG | |
| | LKLALHLAEIPNREKENQMLLSLLPDRIGHGTFLSASEAGA | |
| | LDQVDFVRQHQIPLELCLTSNIKSQTVPSYDQHHFGFWYSI | |
| | AHPSVICTDDKGVFATYLSQEYQLAAETFNLTPFQVWDLS | |
| | YESINYIFACDNTRSELRKRWTHLKQKVLNCNEVNYF | |
| Human ADAD2 | MASASQGADDDGSRRKPRLAASLQISPQPRPWRPLPAQAQ | SEQ ID |
| | SAWGPAPAPATYRAEGGWPQVSVLRDSGPGAGAGVGEL | NO. 249 |
| | GAARAWENLGEQMGKAPRVPVPPAGLSLPLKDPPASQAV | |
| | SLLTEYAASLGIFLLFREDQPPGPCFPFSVSAELDGVVCPAG | |
| | TANSKTEAKQQAALSALCYIRSQLENPESPQTSSRPPLAPLS | |
| | VENILTHEQRCAALVSAGFDLLLDERSPYWACKGTVAGVI | |
| | LEREIPRARGHVKEIYKLVALGTGSSCCAGWLEFSGQQLH | |
| | DCHGLVIARRALLRFLFRQLLLATQGGPKGKEQSVLAPQP | |
| | GPGPPFTLKPRVFLHLYISNTPKGAARDIYLPPTSEGGLPHS | |
| | PPMRLQAHVLGQLKPVCYVAPSLCDTHVGCLSASDKLAR | |
| | WAVLGLGGALLAHLVSPLYSTSLILADSCHDPPTLSRAIHT | |
| | RPCLDSVLGPCLPPPYVRTALHLFAGPPVAPSEPTPDTCRG | |
| | LSLNWSLGDPGIEVVDVATGRVKANAALGPPSRLCKASFL | |
| | RAFHQAARAVGKPYLLALKTYEAAKAGPYQEARRQLSLL | |
| | LDQQGLGAWPSKPLVGKFRN | |
| Mus musculus | MASVDEGGRRRPRLAASLQISPGPWKPSGGQEPTEAGDAA | SEQ ID |
| ADAD2 | PRTAEHGVAGAQEAHREACKALGGSVLSPGPAGDFPGAL | NO. 250 |
| | HGLSMLPKDPPPAQAVALLTQCMANLGVSLTFLEDQTAG | |
| | PGSSFSVCADLDGLVCPAGTGSSKLEAKQQAALSALQYIQ | |
| | KQLERPEPLVTPRQPLLTSLSIETILTHEQRCAAVVSAGLDR | |
| | LLSESSPYQACKGTVAAVILEREVQGSIGHSKETYELVALG | |
| | TGSSSCAGWLEFSGRRLHDCHGLVIARRALLRFFFRQLLLV | |
| | TQGGPKGQERSVLTPQPGPGPPFALKPGVFLHLYVSNTPK | |
| | GAAHDIYLPLASEDSVLHSPAFRLQAHVCGQLKPVSYVAP | |
| | ALRDTHVGCLSASDKLARWAILGLGGGLLAHFLPPLYATS | |
| | LVLADPCHDPSTLNRVIHSRPRLDSVLGSCLPCPYVRTTLH | |
| | LFAGPLVAPSDPGPSTCHSLSLNWSLGDPDIEVVDVATGR | |
| | VKTDSSVGPPSRLCKAAFLSAFRQVARALEKPQLLSLQTY | |
| | EAAKAVPYREARQQLSLLLDQQGLGAWPSKPLVGKFRH | |
| Macaca fascicularis | MASASQGADDGSRRKPRLAASLQISPEPRPWRPLPPQAQG | SEQ ID |
| ADAD2 | AWEPAPAMDHAEGGQPQVSVLRDSGPGAGAGVGELGAA | NO. 251 |
| | QAWENLGEQMGRTPRVPVPPAGLSLPLKDPPASQAVSLLT | |
| | EYAASLGIILLFREDQQPGPCFPFSVSAELDGVVCPAGTAN | |
| | SKTEAKQQAALSALCYIRSQLESPESPQTSSRPPPPPLSVDSI | |
| | LTHGQRCAALVSAGFDLLLDERSPYWACKGTVAGVILERE | |
| | IPGARGHVKEIYKLVALGTGSSCCAGWLEFSGQQLHDCHG | |
| | LVIARRALLRFLFRQLLLATQGGAKGKEQSVLAPQPGPGPP | |
| | FTLKPRVFLHLYISNTPKGAARDIYLPPTSEGGLPHSPPMRL | |
| | QAHVLGQLKPVCYVAPSLCDTHVGCLSASDKLARWAVLG | |
| | LGGALLAHLVSPLYSTSLILADSCHDPPTLSRAIHTRPCLDS | |
| | VLGPCLPPPYVRTALHLFSGPPVAPSEPTPDTCHGLSLNWS | |
| | LGDPGIEVVDVATGRVKANAALGPPSRLCKASFLRAFHQV | |
| | ARAVGKPYLLALKTYEAAKAGPYQEARRQLSLLLDQQGL | |
| | GAWPSKPLVGKFRN | |

TABLE 13
amino acid sequence SEQ ID NO: 252-287

| | | SEQ ID |
|---|---|---|
| Name | Sequence | NO |
| Felis catus | MNPLQEVIFCRQFGNQHRVPKPYYRRKTYLCYQLKLPEGT | SEQ ID |
| APOBEC3H | LIHKDCLRNKKKRHAEMCFIDKIKALTRDTSQRFEIICYITW | NO. 252 |
| | SPCPFCAEELVAFVKDNPHLSLRIFASRLYVHWRWKYQQG | |
| | LRHLHASGIPVAVMSLPEFEDCWRNFVDHQDRSFQPWPNL | |
| | DQYSKSIKRRLGKILTPLNDLRNDFRNLKLE | |
| Human APOBEC3A | MEASPASGPRHLMDPHIFTSNFNNGIGRHKTYLCYEVERL | SEQ ID |
| | DNGTSVKMDQHRGFLHNQAKNLLCGFYGRHAELRFLDLV | NO. 253 |
| | PSLQLDPAQIYRVTWFISWSPCFSWGCAGEVRAFLQENTH | |
| | VRLRIFAARIYDYDPLYKEALQMLRDAGAQVSIMTYDEFK | |
| | HCWDTFVDHQGCPFQPWDGLDEHSQALSGRLRAILQNQG | |
| | N | |
| Cricetulus | MGPFCLGCSHRKCYSPIRNLISQETFKFHFKNLGYAKGRKD | SEQ ID |
| longicaudatus | TFLCYEVTRKDCDSPVSLHHGVFKNKGNIHAEVCFLYWFH | NO. 254 |
| A3 | DKVLKVLSPREEFKITWYMSWSPCFECAEQIVRFLATHHY | |
| | LSLDIFSSRLYNVQDPETQQNLCRLVQEGAQVAAMDLYEF | |
| | KKCWKKFVTMVAGDSGLGKRLLTNFRYQDSKLQEILRRM | |
| | DPLSEEEFYSQFYNQRVKHLCYYHRMKPYLCYQLEQFNG | |
| | QAPLKGCLLSEKGKQHAEILFLDKIRSMELSQVTITCYLTW | |
| | SPCPNCAWRLAAFKRDRPDLILHIYTSRLYFHWKRPFQKG | |
| | LCSLWQSGILVDVMDLPQFTDCWTNFVNPKRPFWPWKGL | |
| | EIISRRTQRRLRRIKESWGLQDLVNDFGNLQLGPPMS | |
| Rattus | MGPFCLGCSHRKCYSPIRNLISQETFKFHFKNLRYAIDRKD | SEQ ID |
| norvegicus | TFLCYEVTRKDCDSPVSLHHGVFKNKDNIHAEICFLYWFH | NO. 255 |
| APOBEC3 | DKVLKVLSPREEFKITWYMSWSPCFECAEQVLRFLATHHN | |
| | LSLDIFSSRLYNIRDPENQQNLCRLVQEGAQVAAMDLYEF | |
| | KKCWKKFVDNGGRRFRPWKKLLTNFRYQDSKLQEILRPC | |
| | YIPVPSSSSSTLSNICLTKGLPETRFCVERRRVHLLSEEEFYS | |
| | QFYNQRVKHLCYYHGVKPYLCYQLEQFNGQAPLKGCLLS | |
| | EKGKQHAEILFLDKIRSMELSQVIITCYLTWSPCPNCAWQL | |
| | AAFKRDRPDLILHIYTSRLYFHWKRPFQKGLCSLWQSGILV | |
| | DVMDLPQFTDCWTNFVNPKRPFWPWKGLEIISRRTQRRLH | |
| | RIKESWGLQDLVNDFGNLQLGPPMS | |
| Pongo pygmaeus | MALLTAKTFSLQFNNKRRIKRPYYPRKALLCYQLTPQNGS | SEQ ID |
| APOBEC3H | TPTRGYFKNKKKCHAEIRFINEIKSMGLDETQCYQVTCYLT | NO. 256 |
| | WSPCPSCVRELVAFIKAHDHLNLRIFASRLYCHWCRRQQE | |
| | GLRLLCGSQVPVEVMGSREFADCWENFVDHEKPLSFNPSE | |
| | MLEELDKNSRAIKRRLERIKQSWSVDVLENGLRSLQLGPVS | |
| | SSLSRSNSR | |
| Felis catus | MEPWRPSPRNPMDRIDPNTFRFHFPNLLYASGRKLCYLCF | SEQ ID |
| APOBEC3Ca | QVETEDYFSCDDSDRGVFRNKVHPWARCHAEQCFLSWFR | NO. 257 |
| | DQYPYRDEYYNVTWFLSWSPCPTCAEEVVEFLEEYRNLTL | |
| | SIFTSRLYYFWDPNYQEGLCKLWDAGVQLDIMSCDDFKHC | |
| | WDNFVDHKGMRFQRRNLLKDYDFLAAELQEILR | |
| Human APOBEC3H | MALLTAETFRLQFNNKRRLRRPYYPRKALLCYQLTPQNGS | SEQ ID |
| | TPTRGYFENKKKCHAEICFINEIKSMGLDETQCYQVTCYLT | NO. 258 |
| | WSPCSSCAWELVDFIKAHDHLNLGIFASRLYYHWCKPQQK | |
| | GLRLLCGSQVPVEVMGFPEFADCWENFVDHEKPLSFNPYK | |
| | MLEELDKNSRAIKRRLERIKIPGVRAQGRYMDILCDAEV | |
| Macaca mulatta | MVEPMDPRTFVSNFNNRPILSGLNTVWLCCEVKTKDPSGP | SEQ ID |
| APOBEC3G | PLDAKIFQGKVYSKAKYHPEMRFLRWFHKWRQLHHDQEY | NO. 259 |
| | KVTWYVSWSPCTRCANSVATFLAKDPKVTLTIFVARLYYF | |
| | WKPDYQQALRILCQKRGGPHATMKIMNYNEFQDCWNKF | |
| | VDGRGKPFKPRNNLPKHYTLLQATLGELLRHLMDPGTFTS | |
| | NFNNKPWVSGQHETYLCYKVERLHNDTWVPLNQHRGFLR | |
| | NQAPNIHGFPKGRHAELCFLDLIPFWKLDGQQYRVTCFTS | |
| | WSPCFSCAQEMAKFISNNEHVSLCIFAARIYDDQGRYQEGL | |
| | RALHRDGAKIAMMNYSEFEYCWDTFVDRQGRPFQPWDG | |
| | LDEHSQALSGRLRAI | |
| Pan troglodytes | MKPHFRNPVERMYQDTFSDNFYNRPILSHRNTVWLCYEV | SEQ ID |
| APOBEC3G | KTKGPSRPPLDAKIFRGQVYSKLKYHPEMRFFHWFSKWRK | NO. 260 |
| | LHRDQEYEVTWYISWSPCTKCTRDVATFLAEDPKVTLTIFV | |
| | ARLYYFWDPDYQEALRSLCQKRDGPRATMKIMNYDEFQH | |
| | CWSKFVYSQRELFEPWNNLPKYYILLHIMLGEILRHSMDPP | |
| | TFTSNFNNELWVRGRHETYLCYEVERLHNDTWVLLNQRR | |
| | GFLCNQAPHKHGFLEGRHAELCFLDVIPFWKLDLHQDYRV | |
| | TCFTSWSPCFSCAQEMAKFISNNKHVSLCIFAARIYDDQGR | |
| | CQEGLRTLAKAGAKISIMTYSEFKHCWDTFVDHQGCPFQP | |
| | WDGLEEHSQALSGRLRAILQNQGN | |
| Chlorocebus | MNPQIRNMVEQMEPDIFVYYFNNRPILSGRNTVWLCYEVK | SEQ ID |
| aethiops | TKDPSGPPLDANIFQGKLYPEAKDHPEMKFLHWFRKWRQ | NO. 261 |
| APOBEC3G | LHRDQEYEVTWYVSWSPCTRCANSVATFLAEDPKVTLTIF | |
| | VARLYYFWKPDYQQALRILCQERGGPHATMKIMNYNEFQ | |
| | HCWNEFVDGQGKPFKPRKNLPKHYTLLHATLGELLRHVM | |
| | DPGTFTSNFNNKPWVSGQRETYLCYKVERSHNDTWVLLN | |
| | QHRGFLRNQAPDRHGFPKGRHAELCFLDLIPFWKLDDQQY | |
| | RVTCFTSWSPCFSCAQKMAKFISNNKHVSLCIFAARIYDDQ | |
| | GRCQEGLRTLHRDGAKIAVMNYSEFEYCWDTFVDRQGRP | |
| | FQPWDGLDEHSQALSGRLRAI | |
| Human APOBEC3F | MKPHFRNTVERMYRDTFSYNFYNRPILSRRNTVWLCYEV | SEQ ID |
| | KTKGPSRPRLDAKIFRGQVYSQPEHHAEMCFLSWFCGNQL | NO. 262 |
| | PAYKCFQITWFVSWTPCPDCVAKLAEFLAEHPNVTLTISAA | |
| | RLYYYWERDYRRALCRLSQAGARVKIMDDEEFAYCWENF | |
| | VYSEGQPFMPWYKFDDNYAFLHRTLKEILRNPMEAMYPHI | |
| | FYFHFKNLRKAYGRNESWLCFTMEVVKHHSPVSWKRGVF | |
| | RNQVDPETHCHAERCFLSWFCDDILSPNTNYEVTWYTSWS | |
| | PCPECAGEVAEFLARHSNVNLTIFTARLYYFWDTDYQEGL | |
| | RSLSQEGASVEIMGYKDFKYCWENFVYNDDEPFKPWKGL | |
| | KYNFLFLDSKLQEILE | |
| Human APOBEC3G | MKPHFRNTVERMYRDTFSYNFYNRPILSRRNTVWLCYEV | SEQ ID |
| | KTKGPSRPPLDAKIFRGQVYSELKYHPEMRFFHWFSKWRK | NO. 263 |
| | LHRDQEYEVTWYISWSPCTKCTRDMATFLAEDPKVTLTIF | |
| | VARLYYFWDPDYQEALRSLCQKRDGPRATMKIMNYDEFQ | |
| | HCWSKFVYSQRELFEPWNNLPKYYILLHIMLGEILRHSMD | |
| | PPTFTFNFNNEPWVRGRHETYLCYEVERMHNDTWVLLNQ | |
| | RRGFLCNQAPHKHGFLEGRHAELCFLDVIPFWKLDLDQDY | |
| | RVTCFTSWSPCFSCAQEMAKFISKNKHVSLCIFTARIYDDQ | |
| | GRCQEGLRTLAEAGAKISIMTYSEFKHCWDTFVDHQGCPF | |
| | QPWDGLDEHSQDLSGRLRAILQNQEN | |
| Human APOBEC3C | MNPQIRNPMKAMYPGTFYFQFKNLWEANDRNETWLCFTV | SEQ ID |
| | EGIKRRSVVSWKTGVFRNQVDSETHCHAERCFLSWFCDDI | NO. 264 |
| | LSPNTKYQVTWYTSWSPCPDCAGEVAEFLARHSNVNLTIF | |
| | TARLYYFQYPCYQEGLRSLSQEGVAVEIMDYEDFKYCWE | |
| | NFVYNDNEPFKPWKGLKTNFRLLKRRLRESLQ | |
| Human APOBEC3B | MNPQIRNPMERMYRDTFYDNFENEPILYGRSYTWLCYEVK | SEQ ID |
| | IKRGRSNLLWDTGVFRGQVYFKPQYHAEMCFLSWFCGNQ | NO. 265 |
| | LPAYKCFQITWFVSWTPCPDCVAKLAEFLSEHPNVTLTISA | |
| | ARLYYYWERDYRRALCRLSQAGARVTIMDYEEFAYCWEN | |
| | FVYNEGQQFMPWYKFDENYAFLHRTLKEILRYLMDPDTFT | |
| | FNFNNDPLVLRRRQTYLCYEVERLDNGTWVLMDQHMGFL | |
| | CNEAKNLLCGFYGRHAELRFLDLVPSLQLDPAQIYRVTWFI | |
| | SWSPCFSWGCAGEVRAFLQENTHVRLRIFAARIYDYDPLY | |
| | KEALQMLRDAGAQVSIMTYDEFEYCWDTFVYRQGCPFQP | |
| | WDGLEEHSQALSGRLRAILQNQGN | |
| Macaca mulatta | MALLTAKTFSLQFNNKRRVNKPYYPRKALLCYQLTPQNGS | SEQ ID |
| APOBEC3H | TPTRGHLKNKKKDHAEIRFINKIKSMGLDETQCYQVTCYL | NO. 266 |
| | TWSPCPSCAGELVDFIKAHRHLNLRIFASRLYYHWRPNYQ | |
| | EGLLLLCGSQVPVEVMGLPEFTDCWENFVDHKEPPSFNPS | |
| | EKLEELDKNSQAIKRRLERIKSRSVDVLENGLRSLQLGPVT | |
| | PSSSIRNSR | |
| Human APOBEC3D | MNPQIRNPMERMYRDTFYDNFENEPILYGRSYTWLCYEVK | SEQ ID |
| | IKRGRSNLLWDTGVFRGPVLPKRQSNHRQEVYFRFENHAE | NO. 267 |
| | MCFLSWFCGNRLPANRRFQITWFVSWNPCLPCVVKVTKFL | |
| | AEHPNVTLTISAARLYYYRDRDWRWVLLRLHKAGARVKI | |
| | MDYEDFAYCWENFVCNEGQPFMPWYKFDDNYASLHRTL | |
| | KEILRNPMEAMYPHIFYFHFKNLLKACGRNESWLCFTMEV | |
| | TKHHSAVFRKRGVFRNQVDPETHCHAERCFLSWFCDDILS | |
| | PNTNYEVTWYTSWSPCPECAGEVAEFLARHSNVNLTIFTA | |
| | RLCYFWDTDYQEGLCSLSQEGASVKIMGYKDFVSCWKNF | |
| | VYSDDEPFKPWKGLQTNFRLLKRRLREILQ | |
| Mus musculus | MQPQRLGPRAGMGPFCLGCSHRKCYSPIRNLISQETFKFHF | SEQ ID |
| APOBEC3 | KNLGYAKGRKDTFLCYEVTRKDCDSPVSLHHGVFKNKDN | NO. 268 |
| | IHAEICFLYWFHDKVLKVLSPREEFKITWYMSWSPCFECAE | |
| | QIVRFLATHHNLSLDIFSSRLYNVQDPETQQNLCRLVQEGA | |
| | QVAAMDLYEFKKCWKKFVDNGGRRFRPWKRLLTNFRYQ | |
| | DSKLQEILRPCYISVPSSSSSTLSNICLTKGLPETRFWVEGRR | |
| | MDPLSEEEFYSQFYNQRVKHLCYYHRMKPYLCYQLEQFN | |
| | GQAPLKGCLLSEKGKQHAEILFLDKIRSMELSQVTITCYLT | |
| | WSPCPNCAWQLAAFKRDRPDLILHIYTSRLYFHWKRPFQK | |
| | GLCSLWQSGILVDVMDLPQFTDCWTNFVNPKRPFWPWKG | |
| | LEIISRRTQRRLRRIKESWGLQDLVNDFGNLQLGPPMS | |
| Rattus | MSSETGPVAVDPTLRRRIEPHEFEVFFDPRELRKETCLLYEI | SEQ ID |
| norvegicus | NWGGRHSIWRHTSQNTNKHVEVNFIEKFTTERYFCPNTRC | NO. 269 |
| APOBEC1 | SITWFLSWSPCGECSRAITEFLSRYPHVTLFIYIARLYHHAD | |
| | PRNRQGLRDLISSGVTIQIMTEQESGYCWRNFVNYSPSNEA | |
| | HWPRYPHLWVRLYVLELYCIILGLPPCLNILRRKQPQLTFF | |
| | TIALQSCHYQRLPPHILWATGLK | |
| Human APOBEC1 | MTSEKGPSTGDPTLRRRIEPWEFDVFYDPRELRKEACLLYE | SEQ ID |
| | IKWGMSRKIWRSSGKNTTNHVEVNFIKKFTSERDFHPSMS | NO. 270 |
| | CSITWFLSWSPCWECSQAIREFLSRHPGVTLVIYVARLFWH | |
| | MDQQNRQGLRDLVNSGVTIQIMRASEYYHCWRNFVNYPP | |
| | GDEAHWPQYPPLWMMLYALELHCIILSLPPCLKISRRWQN | |
| | HLTFFRLHLQNCHYQTIPPHILLATGLIHPSVAWR | |
| Oryctolagus | MASEKGPSNKDYTLRRRIEPWEFEVFFDPQELRKEACLLYE | SEQ ID |
| cuniculus | IKWGASSKTWRSSGKNTTNHVEVNFLEKLTSEGRLGPSTC | NO. 271 |
| APOBEC1 | CSITWFLSWSPCWECSMAIREFLSQHPGVTLIIFVARLFQH | |
| | MDRRNRQGLKDLVTSGVTVRVMSVSEYCYCWENFVNYP | |
| | PGKAAQWPRYPPRWMLMYALELYCIILGLPPCLKISRRHQ | |
| | KQLTFFSLTPQYCHYKMIPPYILLATGLLQPSVPWR | |
| Mus musculus | MSSETGPVAVDPTLRRRIEPHEFEVFFDPRELRKETCLLYEI | SEQ ID |
| APOBEC1 | NWGGRHSVWRHTSQNTSNHVEVNFLEKFTTERYFRPNTR | NO. 272 |
| | CSITWFLSWSPCGECSRAITEFLSRHPYVTLFIYIARLYHHT | |
| | DQRNRQGLRDLISSGVTIQIMTEQEYCYCWRNFVNYPPSN | |
| | EAYWPRYPHLWVKLYVLELYCIILGLPPCLKILRRKQPQLT | |
| | FFTITLQTCHYQRIPPHLLWATGLK | |
| Mesocricetus | MSSETGPVVVDPTLRRRIEPHEFDAFFDQGELRKETCLLYEI | SEQ ID |
| auratus APOBEC1 | RWGGRHNIWRHTGQNTSRHVEINFIEKFTSERYFYPSTRCS | NO. 273 |
| | IVWFLSWSPCGECSKAITEFLSGHPNVTLFIYAARLYHHTD | |
| | QRNRQGLRDLISRGVTIRIMTEQEYCYCWRNFVNYPPSNE | |
| | VYWPRYPNLWMRLYALELYCIHLGLPPCLKIKRRHQYPLT | |
| | FFRLNLQSCHYQRIPPHILWATGFI | |
| Monodelphis | MNSKTGPSVGDATLRRRIKPWEFVAFFNPQELRKETCLLY | SEQ ID |
| domestica | EIKWGNQNIWRHSNQNTSQHAEINFMEKFTAERHFNSSVR | NO. 274 |
| APOBEC1 | CSITWFLSWSPCWECSKAIRKFLDHYPNVTLAIFISRLYWH | |
| | MDQQHRQGLKELVHSGVTIQIMSYSEYHYCWRNFVDYPQ | |
| | GEEDYWPKYPYLWIMLYVLELHCIILGLPPCLKISGSHSNQ | |
| | LALFSLDLQDCHYQKIPYNVLVATGLVQPFVTWR | |
| Pongo pygmaeus | MTSEKGPSTGDPTLRRRIESWEFDVFYDPRELRKETCLLYE | SEQ ID |
| APOBEC1 | IKWGMSRKIWRSSGKNTTNHVEVNFIKKFTSERRFHSSISC | NO. 275 |
| | SITWFLSWSPCWECSQAIREFLSQHPGVTLVIYVARLFWH | |
| | MDQRNRQGLRDLVNSGVTIQIMRASEYYHCWRNFVNYPP | |
| | GDEAHWPQYPPLWMMLYALELHCIILSLPPCLKISRRWQN | |
| | HLAFFRLHLQNCHYQTIPPHILLATGLIHPSVTWR | |
| Bos taurus | MAQKEEAAAAAEPASQNGEEVENLEDPEKLKELIELPPFEI | SEQ ID |
| APOBEC2 | VTGERLPAHYFKFQFRNVEYSSGRNKTFLCYVVEAQSKGG | NO. 276 |
| | QVQASRGYLEDEHATNHAEEAFFNSIMPTFDPALRYMVT | |
| | WYVSSSPCAACADRIVKTLNKTKNLRLLILVGRLFMWEEP | |
| | EIQAALRKLKEAGCRLRIMKPQDFEYIWQNFVEQEEGESK | |
| | AFEPWEDIQENFLYYEEKLADILK | |
| Mus musculus | MAQKEEAAEAAAPASQNGDDLENLEDPEKLKELIDLPPFEI | SEQ ID |
| APOBEC2 | VTGVRLPVNFFKFQFRNVEYSSGRNKTFLCYVVEVQSKGG | NO. 277 |
| | QAQATQGYLEDEHAGAHAEEAFFNTILPAFDPALKYNVT | |
| | WYVSSSPCAACADRILKTLSKTKNLRLLILVSRLFMWEEPE | |
| | VQAALKKLKEAGCKLRIMKPQDFEYIWQNFVEQEEGESKA | |
| | FEPWEDIQENFLYYEEKLADILK | |
| Human APOBEC2 | MAQKEEAAVATEAASQNGEDLENLDDPEKLKELIELPPFEI | SEQ ID |
| | VTGERLPANFFKFQFRNVEYSSGRNKTFLCYVVEAQGKGG | NO. 278 |
| | QVQASRGYLEDEHAAAHAEEAFFNTILPAFDPALRYNVTW | |
| | YVSSSPCAACADRIIKTLSKTKNLRLLILVGRLFMWEEPEIQ | |
| | AALKKLKEAGCKLRIMKPQDFEYVWQNFVEQEEGESKAF | |
| | QPWEDIQENFLYYEEKLADILK | |
| Pongo pygmaeus | MAQKEEAAAATEAASQNGEDLENLDDPEKLKELIELPPFEI | SEQ ID |
| APOBEC2 | VTGERLPANFFKFQFRNVEYSSGRNKTFLCYVVEAQGKGG | NO. 279 |
| | QVQASRGYLEDEHAAAHAEEAFFNTILPAFDPALRYNVTW | |
| | YVSSSPCAACADRIIKTLSKTKNLRLLILVGRLFMWEELEIQ | |
| | DALKKLKEAGCKLRIMKPQDFEYVWQNFVEQEEGESKAF | |
| | QPWEDIQENFLYYEEKLADILK | |
| Human APOBEC4 | MEPIYEEYLANHGTIVKPYYWLSFSLDCSNCPYHIRTGEEA | SEQ ID |
| | RVSLTEFCQIFGFPYGTTFPQTKHLTFYELKTSSGSLVQKGH | NO. 280 |
| | ASSCTGNYIHPESMLFEMNGYLDSAIYNNDSIRHIILYSNNS | |
| | PCNEANHCCISKMYNFLITYPGITLSIYFSQLYHTEMDFPAS | |
| | AWNREALRSLASLWPRVVLSPISGGIWHSVLHSFISGVSGS | |
| | HVFQPILTGRALADRHNAYEINAITGVKPYFTDVLLQTKRN | |
| | PNTKAQEALESYPLNNAFPGQFFQMPSGQLQPNLPPDLRAP | |
| | VVFVLVPLRDLPPMHMGQNPNKPRNIVRHLNMPQMSFQE | |
| | TKDLGRLPTGRSVEIVEITEQFASSKEADEKKKKKGKK | |
| Rattus | MEPLYEEYLTHSGTIVKPYYWLSVSLNCTNCPYHIRTGEEA | SEQ ID |
| norvegicus | RVPYTEFHQTFGFPWSTYPQTKHLTFYELRSSSGNLIQKGL | NO. 281 |
| APOBEC4 | ASNCTGSHTHPESMLFERDGYLDSLIFHDSNIRHIILYSNNS | |
| | PCDEANHCCISKMYNFLMNYPEVTLSVFFSQLYHTENQFP | |
| | TSAWNREALRGLASLWPQVTLSAISGGIWQSILETFVSGISE | |
| | GLTAVRPFTAGRTLTDRYNAYEINCITEVKPYFTDALHSW | |
| | QKENQDQKVWAASENQPLHNTTPAQWQPDMSQDCRTPA | |
| | VFMLVPYRDLPPIHVNPSPQKPRTVVRHLNTLQLSASKVK | |
| | ALRKSPSGRPVKKEEARKGSTRSQEANETNKSKWKKQTLF | |
| | IKSNICHLLEREQKKIGILSSWSV | |
| Mus musculus | MEPLYEEILTQGGTIVKPYYWLSLSLGCTNCPYHIRTGEEA | SEQ ID |
| APOBEC4 | RVPYTEFHQTFGFPWSTYPQTKHLTFYELRSSSKNLIQKGL | NO. 282 |
| | ASNCTGSHNHPEAMLFEKNGYLDAVIFHNSNIRHIILYSNN | |
| | SPCNEAKHCCISKMYNFLMNYPEVTLSVFFSQLYHTEKQFP | |
| | TSAWNRKALQSLASLWPQVTLSPICGGLWHAILEKFVSNIS | |
| | GSTVPQPFIAGRILADRYNTYEINSIIAAKPYFTDGLLSRQK | |
| | ENQNREAWAAFEKHPLGSAAPAQRQPTRGQDPRTPAVLM | |
| | LVSNRDLPPIHVGSTPQKPRTVVRHLNMLQLSSFKVKDVK | |
| | KPPSGRPVEEVEVMKESARSQKANKKNRSQWKKQTLVIK | |
| | PRICRLLER | |
| Macaca | MEPTYEEYLANHGTIVKPYYWLSFSLDCSNCPYHIRTGEEA | SEQ ID |
| fascicularis | RVSLTEFCQIFGFPYGTTYPQTKHLTFYELKTSSGSLVQKG | NO. 283 |
| ADAT1 | HASSCTGNYIHPESMLFEMNGYLDSAIYNNDSIRHIILYCN | |
| | NSPCNEANHCCISKVYNFLITYPGITLSIYFSQLYHTEMDFP | |
| | ASAWNREALRSLASLWPRVVLSPISGGIWHSVLHSFVSGVS | |
| | GSHVFQPILTGRALTDRYNAYEINAITGVKPFFTDVLLHTK | |
| | RNPNTKAQMALESYPLNNAFPGQSFQMTSGIPPDLRAPVV | |
| | FVLLPLRDLPPMHMGQDPNKPRNIIRHLNMPQMSFQETKD | |
| | LERLPTRRSVETVEITERFASSKQAEEKTKKKKGKK | |
| Human AICDA | MDSLLMNRRKFLYQFKNVRWAKGRRETYLCYVVKRRDS | SEQ ID |
| | ATSFSLDFGYLRNKNGCHVELLFLRYISDWDLDPGRCYRV | NO. 284 |
| | TWFTSWSPCYDCARHVADFLRGNPNLSLRIFTARLYFCED | |
| | RKAEPEGLRRLHRAGVQIAIMTFKDYFYCWNTFVENHERT | |
| | FKAWEGLHENSVRLSRQLRRILLPLYEVDDLRDAFRTLGL | |
| Mus musculus | MDSLLMKQKKFLYHFKNVRWAKGRHETYLCYVVKRRDS | SEQ ID |
| AICDA | ATSCSLDFGHLRNKSGCHVELLFLRYISDWDLDPGRCYRV | NO. 285 |
| | TWFTSWSPCYDCARHVAEFLRWNPNLSLRIFTARLYFCED | |
| | RKAEPEGLRRLHRAGVQIGIMTFKDYFYCWNTFVENRERT | |
| | FKAWEGLHENSVRLTRQLRRILLPLYEVDDLRDAFRMLGF | |
| Canis lupus | MDSLLMKQRKFLYHFKNVRWAKGRHETYLCYVVKRRDS | SEQ ID |
| familiaris | ATSFSLDFGHLRNKSGCHVELLFLRYISDWDLDPGRCYRV | NO. 286 |
| (Dog) AICDA | TWFTSWSPCYDCARHVADFLRGYPNLSLRIFAARLYFCED | |
| | RKAEPEGLRRLHRAGVQIAIMTFKDYFYCWNTFVENREKT | |
| | FKAWEGLHENSVRLSRQLRRILLPLYEVDDLRDAFRTLGL | |
| Bos taurus | MDSLLKKQRQFLYQFKNVRWAKGRHETYLCYVVKRRDS | SEQ ID |
| (Bovine) | PTSFSLDFGHLRNKAGCHVELLFLRYISDWDLDPGRCYRV | NO. 287 |
| | TWFTSWSPCYDCARHVADFLRGYPNLSLRIFTARLYFCDK | |
| | ERKAEPEGLRRLHRAGVQIAIMTFKDYFYCWNTFVENHER | |
| | TFKAWEGLHENSVRLSRQLRRILLPLYEVDDLRDAFRTLGL | |

### REFERENCES

Jinek, M. et al. A programmable dual-RNA-guided DNA endonuclease in adaptive bacterial immunity. Science 337, 816-821 (2012).
Cong, L. et al. Multiplex genome engineering using CRISPR/Cas systems. Science 339, 819-823 (2013).
Mali, P. et al. RNA-guided human genome engineering via Cas9. Science 339, 823-826 (2013).
Wang, L. et al. Eliminating base-editor-induced genome-wide and transcriptome-wide off-target mutations. Nat Cell Biol 23, 552-563 (2021).
Rhun, A. et al. Small RNAs in streptococci. RNA Biol 9:4, 414-426 (2012).

1.-24. (canceled)

25. A gene editing system comprising a helper crRNA (hcrRNA) and a main crRNA (mcrRNA), or at least one DNA polynucleotide encoding the hcrRNA and/or the mcrRNA, wherein the hcrRNA comprises a first spacer sequence and a first linker sequence, wherein the first linker sequence comprises a first protein-binding motif, and the mcrRNA comprises a second spacer sequence and a second linker sequence, wherein the second linker sequence optionally comprises a second protein binding motif.

26. The gene editing system of claim 25, wherein the hcrRNA or the mcrRNA is an engineered crRNA; wherein the engineered crRNA comprises a spacer sequence and a linker sequence, wherein the linker sequence comprises at least one protein-binding motif, wherein the protein-binding motif is an RNA aptamer motif or a variant thereof.

27.-28. (canceled)

29. The gene editing system of claim 25, wherein the first linker sequence and the second linker sequence are
a. SEQ ID NO: 1 and SEQ ID NO: 8, respectively; or
b. SEQ ID NO: 1 and SEQ ID NO: 4, respectively; or
c. SEQ ID NO: 2 and SEQ ID NO: 8, respectively; or
d. SEQ ID NO: 2 and SEQ ID NO: 5, respectively; or
e. SEQ ID NO: 3 and SEQ ID NO: 8, respectively; or
f. SEQ ID NO: 3 and SEQ ID NO: 6, respectively; or
g. SEQ ID NO: 1 and SEQ ID NO: 7, respectively; or
h. SEQ ID NO: 1 and SEQ ID NO: 5, respectively; or
i. SEQ ID NO: 3 and SEQ ID NO: 5, respectively; or
j. SEQ ID NO: 1 and SEQ ID NO: 6, respectively; or
k. SEQ ID NO: 2 and SEQ ID NO: 6, respectively; or
l. SEQ ID NO: 3 and SEQ ID NO: 6, respectively; or
m. SEQ ID NO: 3 and SEQ ID NO: 4, respectively; or
n. SEQ ID NO: 149 and SEQ ID NO: 152, respectively; or
o. SEQ ID NO: 150 and SEQ ID NO: 152, respectively; or
p. SEQ ID NO: 151 and SEQ ID NO: 152, respectively; or
q. SEQ ID NO: 149 and SEQ ID NO: 153, respectively; or
r. SEQ ID NO: 150 and SEQ ID NO: 153, respectively; or
s. SEQ ID NO: 151 and SEQ ID NO: 153, respectively.

30. The gene editing system of claim 25, wherein the hcrRNA and the mcrRNA are
a. SEQ ID NO: 21 and SEQ ID NO: 20, respectively; or
b. SEQ ID NO: 13 and SEQ ID NO: 20, respectively; or
c. SEQ ID NO: 13 and SEQ ID NO: 16, respectively; or
d. SEQ ID NO: 14 and SEQ ID NO: 20, respectively; or
e. SEQ ID NO: 14 and SEQ ID NO: 17, respectively; or
f. SEQ ID NO: 15 and SEQ ID NO: 20, respectively; or
g. SEQ ID NO: 15 and SEQ ID NO: 18, respectively; or
h. SEQ ID NO: 13 and SEQ ID NO: 19, respectively; or
i. SEQ ID NO: 13 and SEQ ID NO: 17, respectively; or
j. SEQ ID NO: 15 and SEQ ID NO: 17, respectively; or
k. SEQ ID NO: 13 and SEQ ID NO: 18, respectively; or
l. SEQ ID NO: 14 and SEQ ID NO: 18, respectively; or
m. SEQ ID NO: 15 and SEQ ID NO: 18, respectively; or
n. SEQ ID NO: 15 and SEQ ID NO: 16, respectively; or
o. SEQ ID NO: 154 and SEQ ID NO: 157, respectively; or
p. SEQ ID NO: 155 and SEQ ID NO: 157, respectively; or
q. SEQ ID NO: 156 and SEQ ID NO: 157, respectively; or
r. SEQ ID NO: 154 and SEQ ID NO: 158, respectively; or
s. SEQ ID NO: 155 and SEQ ID NO: 158, respectively; or
t. SEQ ID NO: 156 and SEQ ID NO: 158, respectively; or
u. SEQ ID NO: 302 and SEQ ID NO: 303, respectively; or
v. SEQ ID NO: 304 and SEQ ID NO: 305, respectively; or
w. SEQ ID NO: 306 and SEQ ID NO: 307, respectively; or
x. SEQ ID NO: 364 and SEQ ID NO: 365, respectively; or
y. SEQ ID NO: 366 and SEQ ID NO: 367, respectively; or
z. SEQ ID NO: 368 and SEQ ID NO: 369, respectively; or
aa. SEQ ID NO: 390, and SEQ ID NO: 389, respectively; or
bb. SEQ ID NO: 382, and SEQ ID NO: 389, respectively; or
cc. SEQ ID NO: 382, and SEQ ID NO: 385, respectively; or
dd. SEQ ID NO: 382, and SEQ ID NO: 385, respectively; or
ee. SEQ ID NO: 383, and SEQ ID NO: 389, respectively; or
ff. SEQ ID NO: 383, and SEQ ID NO: 386, respectively; or
gg. SEQ ID NO: 383, and SEQ ID NO: 386, respectively; or
hh. SEQ ID NO: 384, and SEQ ID NO: 389, respectively; or
ii. SEQ ID NO: 384, and SEQ ID NO: 387, respectively; or
jj. SEQ ID NO: 382, and SEQ ID NO: 388, respectively; or
kk. SEQ ID NO: 382, and SEQ ID NO: 388, respectively; or
ll. SEQ ID NO: 382, and SEQ ID NO: 386, respectively; or
mm. SEQ ID NO: 384, and SEQ ID NO: 386, respectively; or
nn. SEQ ID NO: 382, and SEQ ID NO: 386, respectively; or
oo. SEQ ID NO: 384, and SEQ ID NO: 386, respectively; or
pp. SEQ ID NO: 382, and SEQ ID NO: 387, respectively; or
qq. SEQ ID NO: 383, and SEQ ID NO: 387, respectively; or
rr. SEQ ID NO: 383, and SEQ ID NO: 387, respectively; or
ss. SEQ ID NO: 384, and SEQ ID NO: 387, respectively; or
tt. SEQ ID NO: 384, and SEQ ID NO: 385, respectively; or
uu. SEQ ID NO: 370 and SEQ ID NO: 371, respectively; or
vv. SEQ ID NO: 372 and SEQ ID NO: 373, respectively; or
ww. SEQ ID NO: 374 and SEQ ID NO: 375, respectively or
xx. SEQ ID NO: 376 and SEQ ID NO: 377, respectively; or
yy. SEQ ID NO: 378 and SEQ ID NO: 379, respectively; or
zz. SEQ ID NO: 380 and SEQ ID NO: 381, respectively.

31. The gene editing system of claim 25, further comprising a first tracrRNA and a second tracrRNA, wherein the first tracrRNA and second tracrRNA are the same or different.

32. The gene editing system of claim 31, wherein the first tracrRNA and the second tracrRNA each has a sequence of any one of SEQ ID NOs: 10-12 and 22-24.

33. The gene editing system of claim 31, wherein the first linker sequence, the second linker sequence, the first tracrRNA, and the second tracrRNA are
a. SEQ ID NO: 1, SEQ ID NO: 8, SEQ ID NO: 10, and SEQ ID NO: 10, respectively; or
b. SEQ ID NO: 1, SEQ ID NO: 4, SEQ ID NO: 10, and SEQ ID NO: 10, respectively; or
c. SEQ ID NO: 1, SEQ ID NO: 4, SEQ ID NO: 11, and SEQ ID NO: 11, respectively; or
d. SEQ ID NO: 2, SEQ ID NO: 8, SEQ ID NO: 10, and SEQ ID NO: 10, respectively; or
e. SEQ ID NO: 2, SEQ ID NO: 5, SEQ ID NO: 10, and SEQ ID NO: 10, respectively; or
f. SEQ ID NO: 2, SEQ ID NO: 5, SEQ ID NO: 12, and SEQ ID NO: 12, respectively; or
g. SEQ ID NO: 3, SEQ ID NO: 8, SEQ ID NO: 10, and SEQ ID NO: 10, respectively; or
h. SEQ ID NO: 3, SEQ ID NO: 6, SEQ ID NO: 10, and SEQ ID NO: 10, respectively; or
i. SEQ ID NO: 1, SEQ ID NO: 7, SEQ ID NO: 10, and SEQ ID NO: 10, respectively; or
j. SEQ ID NO: 1, SEQ ID NO: 7, SEQ ID NO: 11, and SEQ ID NO: 11, respectively; or
k. SEQ ID NO: 1, SEQ ID NO: 5, SEQ ID NO: 10, and SEQ ID NO: 10, respectively; or
I. SEQ ID NO: 3, SEQ ID NO: 5, SEQ ID NO: 10, and SEQ ID NO: 10, respectively; or
m. SEQ ID NO: 1, SEQ ID NO: 5, SEQ ID NO: 12, and SEQ ID NO: 12, respectively; or
n. SEQ ID NO: 3, SEQ ID NO: 5, SEQ ID NO: 12, and SEQ ID NO: 12, respectively; or
o. SEQ ID NO: 1, SEQ ID NO: 6, SEQ ID NO: 10, and SEQ ID NO: 10, respectively; or
p. SEQ ID NO: 2, SEQ ID NO: 6, SEQ ID NO: 10, and SEQ ID NO: 10, respectively; or
q. SEQ ID NO: 2, SEQ ID NO: 6, SEQ ID NO: 11, and SEQ ID NO: 11, respectively; or
r. SEQ ID NO: 3, SEQ ID NO: 6, SEQ ID NO: 11, and SEQ ID NO: 11, respectively; or
s. SEQ ID NO: 3, SEQ ID NO: 4, SEQ ID NO: 11, and SEQ ID NO: 11, respectively; or
t. SEQ ID NO: 149, SEQ ID NO: 152, SEQ ID NO: 10, and SEQ ID NO: 10, respectively; or
u. SEQ ID NO: 150, SEQ ID NO: 152, SEQ ID NO: 10, and SEQ ID NO: 10, respectively; or
v. SEQ ID NO: 151, SEQ ID NO: 152, SEQ ID NO: 10, and SEQ ID NO: 10, respectively; or
w. SEQ ID NO: 149, SEQ ID NO: 153, SEQ ID NO: 10, and SEQ ID NO: 10, respectively; or
x. SEQ ID NO: 150, SEQ ID NO: 153, SEQ ID NO: 10, and SEQ ID NO: 10, respectively; or
y. SEQ ID NO: 151, SEQ ID NO: 153, SEQ ID NO: 10, and SEQ ID NO: 10, respectively.

34. The gene editing system of claim 31, wherein the hcrRNA, the mcrRNA, the first tracrRNA, and the second tracrRNA are
a. SEQ ID NO: 21, SEQ ID NO: 20, SEQ ID NO: 22, and SEQ ID NO: 22, respectively; or
b. SEQ ID NO: 13, SEQ ID NO: 20, SEQ ID NO: 22, and SEQ ID NO: 22, respectively; or
c. SEQ ID NO: 13, SEQ ID NO: 16, SEQ ID NO: 22, and SEQ ID NO: 22, respectively; or
d. SEQ ID NO: 13, SEQ ID NO: 16, SEQ ID NO: 23, and SEQ ID NO: 23, respectively; or
e. SEQ ID NO: 14, SEQ ID NO: 20, SEQ ID NO: 22, and SEQ ID NO: 22, respectively; or
f. SEQ ID NO: 14, SEQ ID NO: 17, SEQ ID NO: 22, and SEQ ID NO: 22, respectively; or
g. SEQ ID NO: 14, SEQ ID NO: 17, SEQ ID NO: 24, and SEQ ID NO: 24, respectively; or
h. SEQ ID NO: 15, SEQ ID NO: 20, SEQ ID NO: 22, and SEQ ID NO: 22, respectively; or
i. SEQ ID NO: 15, SEQ ID NO: 18, SEQ ID NO: 22, and SEQ ID NO: 22, respectively; or
j. SEQ ID NO: 13, SEQ ID NO: 19, SEQ ID NO: 22, and SEQ ID NO: 22, respectively; or
k. SEQ ID NO: 13, SEQ ID NO: 19, SEQ ID NO: 23, and SEQ ID NO: 23, respectively; or
l. SEQ ID NO: 13, SEQ ID NO: 17, SEQ ID NO: 22, and SEQ ID NO: 22, respectively; or
m. SEQ ID NO: 15, SEQ ID NO: 17, SEQ ID NO: 22, and SEQ ID NO: 22, respectively; or
n. SEQ ID NO: 13, SEQ ID NO: 17, SEQ ID NO: 24, and SEQ ID NO: 24, respectively; or
o. SEQ ID NO: 15, SEQ ID NO: 17, SEQ ID NO: 24, and SEQ ID NO: 24, respectively; or
p. SEQ ID NO: 13, SEQ ID NO: 18, SEQ ID NO: 22, and SEQ ID NO: 22, respectively; or
q. SEQ ID NO: 14, SEQ ID NO: 18, SEQ ID NO: 22, and SEQ ID NO: 22, respectively; or
r. SEQ ID NO: 14, SEQ ID NO: 18, SEQ ID NO: 23, and SEQ ID NO: 23, respectively; or
s. SEQ ID NO: 15, SEQ ID NO: 18, SEQ ID NO: 23, and SEQ ID NO: 23, respectively; or
t. SEQ ID NO: 15, SEQ ID NO: 16, SEQ ID NO: 23, and SEQ ID NO: 23, respectively; or
u. SEQ ID NO: 154, SEQ ID NO: 157, SEQ ID NO: 22, and SEQ ID NO: 22, respectively; or
v. SEQ ID NO: 155, SEQ ID NO: 157, SEQ ID NO: 22, and SEQ ID NO: 22, respectively; or
w. SEQ ID NO: 156, SEQ ID NO: 157, SEQ ID NO: 22, and SEQ ID NO: 22, respectively; or
x. SEQ ID NO: 154, SEQ ID NO: 158, SEQ ID NO: 22, and SEQ ID NO: 22, respectively; or
y. SEQ ID NO: 155, SEQ ID NO: 158, SEQ ID NO: 22, and SEQ ID NO: 22, respectively; or
z. SEQ ID NO: 156, SEQ ID NO: 158, SEQ ID NO: 22, and SEQ ID NO: 22, respectively; or
aa. SEQ ID NO: 302, SEQ ID NO: 303, SEQ ID NO: 22, and SEQ ID NO: 22, respectively; or
bb. SEQ ID NO: 304, SEQ ID NO: 305, SEQ ID NO: 22, and SEQ ID NO: 22, respectively; or
cc. SEQ ID NO: 306, SEQ ID NO: 307, SEQ ID NO: 22, and SEQ ID NO: 22, respectively; or
dd. SEQ ID NO: 364, SEQ ID NO: 365, SEQ ID NO: 22, and SEQ ID NO: 22, respectively; or
ee. SEQ ID NO: 366, SEQ ID NO: 367, SEQ ID NO: 22, and SEQ ID NO: 22, respectively; or
ff. SEQ ID NO: 368, SEQ ID NO: 369, SEQ ID NO: 22, and SEQ ID NO: 22, respectively; or
gg. SEQ ID NO: 390, SEQ ID NO: 389, SEQ ID NO: 10, and SEQ ID NO: 10, respectively; or
hh. SEQ ID NO: 382, SEQ ID NO: 389, SEQ ID NO: 10, and SEQ ID NO: 10, respectively; or
ii. SEQ ID NO: 382, SEQ ID NO: 385, SEQ ID NO: 10, and SEQ ID NO: 10, respectively; or
jj. SEQ ID NO: 382, SEQ ID NO: 385, SEQ ID NO: 11, and SEQ ID NO: 11, respectively; or
kk. SEQ ID NO: 383, SEQ ID NO: 389, SEQ ID NO: 10, and SEQ ID NO: 10, respectively; or
ll. SEQ ID NO: 383, SEQ ID NO: 386, SEQ ID NO: 10, and SEQ ID NO: 10, respectively; or
mm. SEQ ID NO: 383, SEQ ID NO: 386, SEQ ID NO: 12, and SEQ ID NO: 12, respectively; or
nn. SEQ ID NO: 384, SEQ ID NO: 389, SEQ ID NO: 10, and SEQ ID NO: 10, respectively; or
oo. SEQ ID NO: 384, SEQ ID NO: 387, SEQ ID NO: 10, and SEQ ID NO: 10, respectively; or
pp. SEQ ID NO: 382, SEQ ID NO: 388, SEQ ID NO: 10, and SEQ ID NO: 10, respectively; or
qq. SEQ ID NO: 382, SEQ ID NO: 388, SEQ ID NO: 11, and SEQ ID NO: 11, respectively; or
rr. SEQ ID NO: 382, SEQ ID NO: 386, SEQ ID NO: 10, and SEQ ID NO: 10, respectively; or
ss. SEQ ID NO: 384, SEQ ID NO: 386, SEQ ID NO: 10, and SEQ ID NO: 10, respectively; or
tt. SEQ ID NO: 382, SEQ ID NO: 386, SEQ ID NO: 12, and SEQ ID NO: 12, respectively; or
uu. SEQ ID NO: 384, SEQ ID NO: 386, SEQ ID NO: 12, and SEQ ID NO: 12, respectively; or
vv. SEQ ID NO: 382, SEQ ID NO: 387, SEQ ID NO: 10, and SEQ ID NO: 10, respectively; or
ww. SEQ ID NO: 383, SEQ ID NO: 387, SEQ ID NO: 10, and SEQ ID NO: 10, respectively; or
xx. SEQ ID NO: 383, SEQ ID NO: 387, SEQ ID NO: 11, and SEQ ID NO: 11, respectively; or
yy. SEQ ID NO: 384, SEQ ID NO: 387, SEQ ID NO: 11, and SEQ ID NO: 11, respectively; or
zz. SEQ ID NO: 384, SEQ ID NO: 385, SEQ ID NO: 11, and SEQ ID NO: 11, respectively; or
aaa. SEQ ID NO: 370, SEQ ID NO: 371, SEQ ID NO: 10, and SEQ ID NO: 10, respectively; or
bbb. SEQ ID NO: 372, SEQ ID NO: 373, SEQ ID NO: 10, and SEQ ID NO: 10, respectively; or
ccc. SEQ ID NO: 374, SEQ ID NO: 375, SEQ ID NO: 10, and SEQ ID NO: 10, respectively or
ddd. SEQ ID NO: 376, SEQ ID NO: 377, SEQ ID NO: 10, and SEQ ID NO: 10, respectively; or
eee. SEQ ID NO: 378, SEQ ID NO: 379, SEQ ID NO: 10, and SEQ ID NO: 10, respectively; or
fff. SEQ ID NO: 380, SEQ ID NO: 381, SEQ ID NO: 10, and SEQ ID NO: 10, respectively.

35. The gene editing system of claim 25, comprising
a. the hcrRNA comprising a first spacer sequence and a first linker sequence, wherein the first linker sequence comprises a first protein-binding motif,
b. the mcrRNA comprising a second spacer sequence and a second linker sequence, wherein the second linker sequence comprises a second protein-binding motif,
c. a first tracrRNA which is capable of forming a first base-pair structure with the hcrRNA,
d. a second tracrRNA which is capable of forming a second base-pair structure with the mcrRNA,
e. a first CRISPR-associated protein (Cas protein), or a polynucleotide encoding the first Cas protein, wherein the first Cas protein binds to the first base-pair structure,
f. a second Cas protein, or a polynucleotide encoding the second Cas protein, wherein the second Cas protein binds to the second base pair structure, and
g. a first fusion protein comprising a nucleobase deaminase or a catalytic domain thereof and a first RNA binding domain, or a polynucleotide encoding the first fusion protein, wherein the nucleobase deaminase or the catalytic domain thereof and the first RNA binding domain are optionally connected by a linker, and wherein the first RNA binding domain binds to the first protein-binding motif,
wherein the first Cas protein and the second Cas protein are the same or different, and the first tracrRNA and the second tracrRNA are the same or different.

36. The gene editing system of claim 35, further comprising
a. a protease, or a polynucleotide encoding the protease, and
b. a nucleobase deaminase inhibitor domain,
wherein the nucleobase deaminase inhibitor domain is connected to the nucleobase deaminase or the catalytic domain thereof in the first fusion protein optionally by a linker, and wherein there is a cleavage site for the protease between the nucleobase deaminase inhibitor domain and the nucleobase deaminase or the catalytic domain thereof.

37. The gene editing system of claim 36, further comprising
a second fusion protein comprising the protease and a second RNA binding domain, or a polynucleotide encoding the second fusion protein,
wherein the protease and the second RNA binding domain are optionally connected by a linker,
and wherein the second RNA binding domain binds to the second protein-binding motif.

38. The gene editing system of claim 36, wherein the protease is split into a first protease fragment and a second protease fragment, wherein the first and/or second protease fragment alone is not able to cleave the cleavage site.

39. The gene editing system of claim 38, further comprising
a. a second fusion protein comprising the first protease fragment and a second RNA binding domain, or a polynucleotide encoding the second fusion protein, wherein the first protease fragment and the second RNA binding domain are optionally connected by a linker, and
b. a third fusion protein comprising the second protease fragment and a third RNA binding domain, or a polynucleotide encoding the third fusion protein, wherein the second protease fragment and the third RNA binding domain are optionally connected by a linker,
wherein the mcrRNA further comprises a third protein-binding motif,
wherein the second RNA binding domain binds to the second protein-binding motif, and
wherein the third RNA binding domain binds to the third protein-binding motif.

40. The gene editing system of claim 39, wherein the second and the third RNA binding domains are the same or different, and the second and the third protein-binding motifs are the same or different.

41. The gene editing system of claim 38, further comprising
a second fusion protein comprising the first protease fragment and a second RNA binding domain, or a polynucleotide encoding the second fusion protein,
wherein the first protease fragment and the second RNA binding domain are optionally connected by a linker,
wherein the second RNA binding domain binds to the second protein-binding motif.

42. The gene editing system of claim 36, wherein the protease is a TEV protease, a TuMV protease, a PPV protease, a PVY protease, a ZIKV protease, or a WNV protease.

43.-58. (canceled)

59. The gene editing system in claim 35, wherein the first fusion protein comprises one or more nucleotide deaminase, and the one or more nucleotide deaminase are the same or different.

60. The gene editing system of claim 59, wherein each of the one or more nucleotide deaminase is a cytidine deaminase or an adenosine deaminase; and/or wherein the nucleotide deaminase is a fusion of at least one cytidine deaminase and at least one adenosine deaminase.

61. (canceled)

62. The gene editing system of claim 35, wherein the first fusion protein further comprises one or more copies of uracil glycosylase inhibitor (UGI); and/or wherein each of the Cas protein is a Cas9, a dead Cas9 (dCas9), or a Cas9 nickase (nCas9) selected from the group consisting of SpCas9, FnCas9, St1Cas9, St3Cas9, NmCas9, SaCas9, AsCpfl, LbCpfl, FnCpfl, VQR Cas9, EQR Cas9, VRER Cas9, Cas9-NG, xCas9, eCas9, SpCas9-HF1, HypaCas9, HiFiCas9, sniper-Cas9, SpG, SpRY, KKH SaCas9, CjCas9, Cas9-NRRH, Cas9-NRCH, Cas9-NRTH, SsCpfl, PcCDfl, BDCDfl, LiCpfl, PmCpfl, Lb2Cpf1, PbCpfl, PbCpfl, PeCuf1, PdCpf1, MbCf1, EeCuf1, CmtCcof1, BsCpfl, BhCasl2b, AkCasl2b, BsCasl2b, AmCasl2b, AaCasl2b, RfxCasl3d, LwaCasl3a, PspCasl3b, PquCasl3b, and RanCasl3b.

63. (canceled)

64. The gene editing system of claim 35, wherein at least one of the tracrRNA is selected from SEQ ID NOs: 10-12 and 22-24.

65. The gene editing system of claim 35, wherein the first protein-binding RNA motif and the first RNA binding domain, the second protein-binding RNA motif and the second RNA binding domain, and the third protein-binding RNA motif and the third RNA binding domain, are each independently selected from the group consisting of
a MS2 phage operator stem-loop and MS2 coat protein (MCP) or an RNA-binding section thereof,
a boxB and N22p or an RNA-binding section thereof,
a telomerase Ku binding motif and Ku protein or an RNA-binding section thereof,
a telomerase Sm7 binding motif and Sm7 protein or an RNA-binding section thereof,
a PP7 phage operator stem-loop and PP7 coat protein (PCP) or an RNA-binding section thereof,
a SfMu phage Com stem-loop and Com RNA binding protein or an RNA-binding section thereof, and
an RNA aptamer and corresponding aptamer ligand or an RNA-binding section thereof.

66. (canceled)

67. A polynucleotide comprising a sequence encoding all components except the first and second Cas proteins in the gene editing system of claim 35.

68. A kit comprising a polynucleotide comprising a sequence encoding all components except the first and second Cas proteins in the gene editing system of claim 35, and a polynucleotide encoding the first and/or the second Cas protein of claim 35.

69. (canceled)

70. A vector comprising the polynucleotide of claim 67.

71.-72. (canceled)

73. A kit comprising a vector comprising a polynucleotide comprising a sequence encoding all components except the first and second Cas proteins in the gene editing system of claim 35, and a vector comprising the polynucleotide encoding the first and/or second Cas protein of claim 35.

74. (canceled)

75. A cell comprising the gene editing system of claim 25.

76.-82. (canceled)

83. A method for reducing low-density lipoprotein cholesterol (LDL-C) in a subject by editing the PCSK9 gene in the subject, comprising administering to the subject the gene editing system of claim 25, wherein the hcrRNA and the mcrRNA are
a. SEQ ID NO: 302 and SEQ ID NO: 303, respectively; or
b. SEQ ID NO: 304 and SEQ ID NO: 305, respectively; or
c. SEQ ID NO: 306 and SEQ ID NO: 307, respectively; or
d. SEQ ID NO: 370 and SEQ ID NO: 371, respectively; or
e. SEQ ID NO: 372 and SEQ ID NO: 373, respectively; or
f. SEQ ID NO: 374 and SEQ ID NO: 375, respectively.

84. A method for reducing low-density lipoprotein cholesterol (LDL-C) and triglyceride in a subject by editing the ANGPTL3 gene in the subject, comprising administering to the subject the gene editing system of claim 25, wherein the hcrRNA and the mcrRNA are
a. SEQ ID NO: 364 and SEQ ID NO: 365, respectively; or
b. SEQ ID NO: 366 and SEQ ID NO: 367, respectively; or
c. SEQ ID NO: 368 and SEQ ID NO: 369, respectively; or
d. SEQ ID NO: 376 and SEQ ID NO: 377, respectively; or
e. SEQ ID NO: 378 and SEQ ID NO: 379, respectively; or
f. SEQ ID NO: 380 and SEQ ID NO: 381, respectively.

85. The engineered crRNA of claim 26, wherein the protein-binding motif is MS2, PP7, boxB, SfMu hairpin motif, telomerase Ku, or Sm7 binding motif.

86. The engineered crRNA of claim 26, wherein the linker sequence is any one of SEQ ID NOs: 1-3 and 149-151; and/or wherein the linker sequence is any one of SEQ ID NOs: 4-7 and 152-153.


SEQUENCE LISTING (395 sequences)

SEQ ID NO: 1 - RNA, 56 nt, synthetic construct
  source 1..56: mol_type = other RNA
gtttgagagctaggccaacatgaggatcacccatgtctgcagggcctgctgttttg

SEQ ID NO: 2 - RNA, 56 nt, synthetic construct
  source 1..56: mol_type = other RNA
gtttgagagctatgctgggccaacatgaggatcacccatgtctgcagggccttttg

SEQ ID NO: 3 - RNA, 56 nt, synthetic construct
  source 1..56: mol_type = other RNA
gtttgagagctatgctgttttgggccaacatgaggatcacccatgtctgcagggcc

SEQ ID NO: 4 - RNA, 41 nt, synthetic construct
  source 1..41: mol_type = other RNA
gtttgagagctagggccctgaagaagggccctgctgttttg

SEQ ID NO: 5 - RNA, 41 nt, synthetic construct
  source 1..41: mol_type = other RNA
gtttgagagctatgctggggccctgaagaagggcccttttg

SEQ ID NO: 6 - RNA, 41 nt, synthetic construct
  source 1..41: mol_type = other RNA
gtttgagagctatgctgttttggggccctgaagaagggccc

SEQ ID NO: 7 - RNA, 62 nt, synthetic construct
  source 1..62: mol_type = other RNA
gtttgagagctagggccctgaagaagggcgggccctgaagaagggcccaacctgctgttttg

SEQ ID NO: 8 - RNA, 22 nt, synthetic construct
  source 1..22: mol_type = other RNA
gtttgagagctatgctgttttg

SEQ ID NO: 9 - RNA, 22 nt, synthetic construct
  source 1..22: mol_type = other RNA
gtttgagagctatgctgttttg

SEQ ID NO: 10 - RNA, 86 nt, synthetic construct
  source 1..86: mol_type = other RNA
ggaaccattcaaaacagcatagcaagttcaaataaggctagtccgttatcaacttgaaaaagtggcaccgagtcggtgcttttttt

SEQ ID NO: 11 - RNA, 89 nt, synthetic construct
  source 1..89: mol_type = other RNA
ggaaccattcaaaacagcaaaatagcaagttcaaataaggctagtccgttatcaacttgaaaaagtggcaccgagtcggtgcttttttt

SEQ ID NO: 12 - RNA, 89 nt, synthetic construct
  source 1..89: mol_type = other RNA
ggaaccattcaaaaaaacagcatagcaagttcaaataaggctagtccgttatcaacttgaaaaagtggcaccgagtcggtgcttttttt

SEQ ID NO: 13 - RNA, 66 nt, synthetic construct
  source 1..66: mol_type = other RNA
  modified_base 1..3: mod_base = OTHER; note = 2'-O-methyl
  modified_base 64..66: mod_base = OTHER; note = 2'-O-methyl
  modified_base 1..4: mod_base = OTHER; note = phosphorothioate linkage
  modified_base 63..66: mod_base = OTHER; note = phosphorothioate linkage
actccacccagtttgagagctaggccaacatgaggatcacccatgtctgcagggcctgctgttttg

SEQ ID NO: 14 - RNA, 66 nt, synthetic construct
  source 1..66: mol_type = other RNA
  modified_base 1..3: mod_base = OTHER; note = 2'-O-methyl
  modified_base 64..66: mod_base = OTHER; note = 2'-O-methyl
  modified_base 1..4: mod_base = OTHER; note = phosphorothioate linkage
  modified_base 63..66: mod_base = OTHER; note = phosphorothioate linkage
actccacccagtttgagagctatgctgggccaacatgaggatcacccatgtctgcagggccttttg

SEQ ID NO: 15 - RNA, 66 nt, synthetic construct
  source 1..66: mol_type = other RNA
  modified_base 1..3: mod_base = OTHER; note = 2'-O-methyl
  modified_base 64..66: mod_base = OTHER; note = 2'-O-methyl
  modified_base 1..4: mod_base = OTHER; note = phosphorothioate linkage
  modified_base 63..66: mod_base = OTHER; note = phosphorothioate linkage
actccacccagtttgagagctatgctgttttgggccaacatgaggatcacccatgtctgcagggcc

SEQ ID NO: 16 - RNA, 61 nt, synthetic construct
  source 1..61: mol_type = other RNA
  modified_base 1..3: mod_base = OTHER; note = 2'-O-methyl
  modified_base 1..4: mod_base = OTHER; note = phosphorothioate linkage
  modified_base 59..61: mod_base = OTHER; note = 2'-O-methyl
  modified_base 58..61: mod_base = OTHER; note = phosphorothioate linkage
cttgaccaatagccttgacagtttgagagctagggccctgaagaagggccctgctgttttg

SEQ ID NO: 17 - RNA, 61 nt, synthetic construct
  source 1..61: mol_type = other RNA
  modified_base 1..3: mod_base = OTHER; note = 2'-O-methyl
  modified_base 1..4: mod_base = OTHER; note = phosphorothioate linkage
  modified_base 59..61: mod_base = OTHER; note = 2'-O-methyl
  modified_base 58..61: mod_base = OTHER; note = phosphorothioate linkage
cttgaccaatagccttgacagtttgagagctatgctggggccctgaagaagggcccttttg

SEQ ID NO: 18 - RNA, 61 nt, synthetic construct
  source 1..61: mol_type = other RNA
  modified_base 1..3: mod_base = OTHER; note = 2'-O-methyl
  modified_base 1..4: mod_base = OTHER; note = phosphorothioate linkage
  modified_base 59..61: mod_base = OTHER; note = 2'-O-methyl
  modified_base 58..61: mod_base = OTHER; note = phosphorothioate linkage
cttgaccaatagccttgacagtttgagagctatgctgttttggggccctgaagaagggccc

SEQ ID NO: 19 - RNA, 82 nt, synthetic construct
  source 1..82: mol_type = other RNA
  modified_base 1..3: mod_base = OTHER; note = 2'-O-methyl
  modified_base 1..4: mod_base = OTHER; note = phosphorothioate linkage
  modified_base 80..82: mod_base = OTHER; note = 2'-O-methyl
  modified_base 79..82: mod_base = OTHER; note = phosphorothioate linkage
cttgaccaatagccttgacagtttgagagctagggccctgaagaagggcgggccctgaagaagggcccaacctgctgttttg

SEQ ID NO: 20 - RNA, 42 nt, synthetic construct
  source 1..42: mol_type = other RNA
  modified_base 1..3: mod_base = OTHER; note = 2'-O-methyl
  modified_base 1..4: mod_base = OTHER; note = phosphorothioate linkage
  modified_base 40..42: mod_base = OTHER; note = 2'-O-methyl
  modified_base 39..42: mod_base = OTHER; note = phosphorothioate linkage
cttgaccaatagccttgacagtttgagagctatgctgttttg

SEQ ID NO: 21 - RNA, 32 nt, synthetic construct
  source 1..32: mol_type = other RNA
  modified_base 1..3: mod_base = OTHER; note = 2'-O-methyl
  modified_base 1..4: mod_base = OTHER; note = phosphorothioate linkage
  modified_base 29..32: mod_base = OTHER; note = phosphorothioate linkage
  modified_base 30..32: mod_base = OTHER; note = 2'-O-methyl
actccacccagtttgagagctatgctgttttg

SEQ ID NO: 22 - RNA, 86 nt, synthetic construct; identical to SEQ ID NO: 10
  source 1..86: mol_type = other RNA
  modified_base 1..3: mod_base = OTHER; note = 2'-O-methyl
  modified_base 1..4: mod_base = OTHER; note = phosphorothioate linkage
  modified_base 83..86: mod_base = OTHER; note = phosphorothioate linkage
  modified_base 84..86: mod_base = OTHER; note = 2'-O-methyl

SEQ ID NO: 23 - RNA, 89 nt, synthetic construct; identical to SEQ ID NO: 11
  source 1..89: mol_type = other RNA
  modified_base 1..3: mod_base = OTHER; note = 2'-O-methyl
  modified_base 1..4: mod_base = OTHER; note = phosphorothioate linkage
  modified_base 87..89: mod_base = OTHER; note = 2'-O-methyl
  modified_base 86..89: mod_base = OTHER; note = phosphorothioate linkage

SEQ ID NO: 24 - RNA, 89 nt, synthetic construct; identical to SEQ ID NO: 12
  source 1..89: mol_type = other RNA
  modified_base 1..3: mod_base = OTHER; note = 2'-O-methyl
  modified_base 1..4: mod_base = OTHER; note = phosphorothioate linkage
  modified_base 87..89: mod_base = OTHER; note = 2'-O-methyl
  modified_base 86..89: mod_base = OTHER; note = phosphorothioate linkage

SEQ ID NO: 25 - protein, 237 aa, Tobacco etch virus
MGESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMVKPEEPFQPVKEATQLMN

SEQ ID NO: 26 - protein, 119 aa, Tobacco etch virus
MGESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQT

SEQ ID NO: 27 - protein, 116 aa, Tobacco etch virus
MKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMVKPEEPFQPVKEATQ

SEQ ID NO: 28 - protein, 7 aa, Tobacco etch virus
ENLYFQS

SEQ ID NO: 29 - protein, 246 aa, Turnip mosaic virus
MASSNSMFRGLRDYNPISNNICHLTNVSDGASNSLYGVGFGPLILTNRHLFERNNGELVIKSRHGEFVIKNTTQLHLLPIPDRDLLLIRLPKDVPPFPQKLGFRQPEKGERICMVGSNFQTKSITSIVSETSTIMPVENSQFWKHWISTKDGQCGSPMVSTKDGKILGLHSLANFQNSINYFAAFPDDFAEKYLHTIEAHEWVKHWKYNTSAISWGSLNIQASQPSGLFKVSKLISDLDSTAVYAQ

SEQ ID NO: 30 - protein, 7 aa, synthetic construct
GGCSHQS

SEQ ID NO: 31 - protein, 246 aa, Plum pox virus
MASSKSLFRGLRDYNPIASSICQLNNSSGARQSEMFGLGFGGLIVTNQHLFKRNDGELTIRSHHGEFVVKDTKTLKLLPCKGRDIVIIRLPKDFPPFPRRLQFRTPTTEDRVCLIGSNFQTKSISSTMSETSATYPVDNSHFWKHWISTKDGHCGLPIVSTRDGSILGLHSLANSTNTQNFYAAFPDNFETTYLSNQDNDNWIKQWRYNPDEVCWGSLQLKRDIPQSPFTICKLLTDLDGEFVYTQ

SEQ ID NO: 32 - protein, 8 aa, synthetic construct
QVVVHQSK

SEQ ID NO: 33 - protein, 247 aa, Potato virus Y
MASAKSLMRGLRDFNPIAQTVCRLKVSVEYGASEMYGFGFGAYIVANHHLFRSYNGSMEVQSMHGTFRVKNLHSLSVLPIKGRDIILIKMPKDFPVFPQKLHFRAPTQNERICLVGTNFQEKYASSIITETSTTYNIPGSTFWKHWIETDNGHCGLPVVSTADGCIVGIHSLANNAHTTNYYSAFDEDFESKYLRTNEHNEWVKSWVYNPDTVLWGPLKLKDSTPKGLFKTTKLVQDLIDHDVVVEQ

SEQ ID NO: 34 - protein, 8 aa, synthetic construct
YDVRHQSR

SEQ ID NO: 35 - protein, 239 aa, Zika virus
MASDMYIERAGDITWEKDAEVTGNSPRLDVALDESGDFSLVEEDGPPMREGGGGSGGGGSGALWDVPAPKEVKKGETTDGVYRVMTRRLLGSTQVGVGVMQEGVFHTMWHVTKGAALRSGEGRLDPYWGDVKQDLVSYCGPWKLDAAWDGLSEVQLLAVPPGERARNIQTLPGIFKTKDGDIGAVALDYPAGTSGSPILDKCGRVIGLYGNGVVIKNGSYVSAITQGKREEETPVECFE

SEQ ID NO: 36 - protein, 8 aa, synthetic construct
KERKRRGA

SEQ ID NO: 37 - protein, 243 aa, West Nile virus
MASSTDMWIERTADISWESDAEITGSSERVDVRLDDDGNFQLMNDPGAPWKGGGGSGGGGGVLWDTPSPKEYKKGDTTTGVYRIMTRGLLGSYQAGAGVMVEGVFHTLWHTTKGAALMSGEGRLDPYWGSVKEDRLCYGGPWKLQHKWNGQDEVQMIVVEPGKNVKNVQTKPGVFKTPEGEIGAVTLDFPTGTSGSPIVDKNGDVIGLYGNGVIMPNGSYISAIVQGERMDEPIPAGFEPEML

SEQ ID NO: 38 - protein, 8 aa, synthetic construct
KQKKRGGK

SEQ ID NO: 39 - protein, 22 aa, Enterovirus E
  source 1..22: note = Porcine teschovirus
GSGATNFSLLKQAGDVEENPGP

SEQ ID NO: 40 - protein, 21 aa, Thosea asigna virus
GSGEGRGSLLTCGDVEENPGP

SEQ ID NO: 41 - protein, 23 aa, Equine rhinitis A virus
GSGQCTNYALLKLAGDVESNPGP

SEQ ID NO: 42 - protein, 223 aa, Mus musculus
MSSSTLSNICLTKGLPETRFWVEGRRMDPLSEEEFYSQFYNQRVKHLCYYHRMKPYLCYQLEQFNGQAPLKGCLLSEKGKQHAEILFLDKIRSMELSQVTITCYLTWSPCPNCAWQLAAFKRDRPDLILHIYTSRLYFHWKRPFQKGLCSLWQSGILVDVMDLPQFTDCWTNFVNPKRPFWPWKGLEIISRRTQRRLRRIKESWGLQDLVNDFGNLQLGPPMS

SEQ ID NO: 43 - protein, 166 aa, Homo sapiens
MNPQIRNPMERMYRDTFYDNFENEPILYGRSYTWLCYEVKIKRGRSNLLWDTGVFRGQVYFKPQYHAEMCFLSWFCGNQLPAYKCFQITWFVSWTPCPDCVAKLAEFLSEHPNVTLTISAARLYYYWERDYRRALCRLSQAGARVKIMDYEEFAYCWENFVYNEGQ

SEQ ID NO: 44 - protein, 207 aa, Mus musculus
MGPFCLGCSHRKCYSPIRNLISQETFKFHFKNLGYAKGRKDTFLCYEVTRKDCDSPVSLHHGVFKNKDNIHAEICFLYWFHDKVLKVLSPREEFKITWYMSWSPCFECAEQIVRFLATHHNLSLDIFSSRLYNVQDPETQQNLCRLVQEGAQVAAMDLYEFKKCWKKFVDNGGRRFRPWKRLLTNFRYQDSKLQEILRPCYISVPSS

SEQ ID NO: 45 - protein, 217 aa, Homo sapiens
MQFMPWYKFDENYAFLHRTLKEILRYLMDPDTFTFNFNNDPLVLRRRQTYLCYEVERLDNGTWVLMDQHMGFLCNEAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFEYCWDTFVYRQGCPFQPWDGLEEHSQALSGRLRAILQNQGN

SEQ ID NO: 46 - protein, 83 aa, Bacillus sp.
  source 1..83: note = Bacillus subtilis bacteriophage PBS1
TNLSDIIEKETGKQLVIQESILMLPEEVEEVIGNKPESDILVHTAYDESTDENVMLLTSDAPEYKPWALVIQDSNGENKIKML

SEQ ID NO: 47 - protein, 1368 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 48 - protein, 117 aa, Escherichia coli
  source 1..117: note = Bacteriophage MS2
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 49 - protein, 123 aa, Pseudomonas aeruginosa
  source 1..123: note = Bacteriophage PP7
MGSKTIVLSVGEATRTLTEIQSTADRQIFEEKVGPLVGRLRLTASLRQNGAKTAYRVNLKLDQADVVDSGLPKVRYTQVWSHDVTIVANSTEASRKSLYDLTKSLVATSQVEDLVVNLVPLGR

SEQ ID NO: 50 - protein, 23 aa, synthetic construct
MGNARTRRRERRAEKQAQWKAAN

SEQ ID NO: 51 - protein, 74 aa, Mus musculus
SEKGKQHAEILFLDKIRSMELSQVTITCYLTWSPCPNCAWQLAAFKRDRPDLILHIYTSRLYFHWKRPFQKGLC

SEQ ID NO: 52 - protein, 74 aa, Mus spicilegus
SEKGKQHAEILFLDKIRSMELSQVTITCYLTWSPCPNCAWQLAAFKRDRPDLIPHIYTSRLYFHWKRPFQKGLC

SEQ ID NO: 53 - protein, 74 aa, Cricetulus longicaudatus
SEKGKQHAEILFLDKIRSMELSQVTITCYLTWSPCPNCAWRLAAFKRDRPDLILHIYTSRLYFHWKRPFQKGLC

SEQ ID NO: 54 - protein, 74 aa, Mus terricolor
SEKGKQHAEILFLNKIRSMELSQVTITCYLTWSPCPNCAWQLAAFKKDRPDLILHIYTSRLYFHWKRPFQKGLC

SEQ ID NO: 55 - protein, 74 aa, Mus caroli
SKKGKQHAEILFLDKIRSMELSQVTITCYLTWSPCPNCAWQLAAFKRDHPDLILHIYTSRLYFHWKRPFQKGLC

SEQ ID NO: 56 - protein, 74 aa, Mus pahari
SKKGKQHAEILFLEKIRSMELSQMRITCYLTWSPCPNCAWQLAAFQKDRPDLILHIYTSRLYFHWRRIFQKGLC

SEQ ID NO: 57 - protein, 74 aa, Mus shortridgei; identical to SEQ ID NO: 56

SEQ ID NO: 58 - protein, 74 aa, Mus setulosus
SKKGKQHAEILFLDKIRSMELSQVRITCYLTWSPCPNCAWQLETFKKDRPDLILHIYTSRLYFHWKRAFQEGLC

SEQ ID NO: 59 - protein, 75 aa, Grammomys sp.
  source 1..75: note = Grammomys surdaster
SKKGKPHAEILFLDKMWSMEELSQVRITCYLTWSPCPNCARQLAAFKKDHPGLILRIYTSRLYFYWRRKFQKGLC

SEQ ID NO: 60 - protein, 73 aa, Rattus norvegicus
KKGEQHVEILFLEKMRSMELSQVRITCYLTWSPCPNCARQLAAFKKDHPDLILRIYTSRLYFYWRKKFQKGLC

SEQ ID NO: 61 - protein, 74 aa, Mastomys coucha
SKKGRQHAEILFLEKVRSMQLSQVRITCYLTWSPCPNCAWQLAAFKMDHPDLILRIYASRLYFHWRRAFQKGLC

SEQ ID NO: 62 - protein, 73 aa, Cricetulus sp.
  source 1..73: note = Cricetulus griseus
NKKGKHAEILFIDEMRSLELGQVQITCYLTWSPCPNCAQELAAFKSDHPDLVLRIYTSRLYFHWRRKYQEGLC

SEQ ID NO: 63 - protein, 73 aa, Peromyscus leucopus
NKKGKHAEILFIDEMRSLELGQARITCYLTWSPCPNCAQKLAAFKKDHPDLVLRVYTSRLYFHWRRKYQEGLC

SEQ ID NO: 64 - protein, 73 aa, Mesocricetus auratus
NKKDKHAEILFIDKMRSLELCQVRITCYLTWSPCPNCAQELAAFKKDHPDLVLRIYTSRLYFHWRRKYQEGLC

SEQ ID NO: 65 - protein, 73 aa, Microtus ochrogaster
NKKGKHAEILFIDEMRSLKLSQERITCYLTWSPCPNCAQELAAFKRDHPGLVLRIYASRLYFHWRRKYQEGLC

SEQ ID NO: 66 - protein, 72 aa, Rhizomys sp.
  source 1..72: note = Nannospalax galili
NKRAKHAEILLIDMMRSMELGQVQITCYITWSPCPTCAQELAAFKQDHPDLVLRIYASRLYFHWKRKFQKGL

SEQ ID NO: 67 - protein, 73 aa, Meriones unguiculatus
NKKGRHAEICLIDEMRSLGLGKAQITCYLTWSPCRKCAQELATFKKDHPDLVLRVYASRLYFHWSRKYQQGLC

SEQ ID NO: 68 - protein, 75 aa, Dipodomys ordii
NKKGHHAEIRFIERIRSMGLDPSQDYQITCYLTWSPCLDCAFKLAKLKKDFPRLTLRIFTSRLYFHWIRKFQKGL

SEQ ID NO: 69 - protein, 72 aa, Jaculus jaculus
NKKGKHAEARFVDKMRSMQLDHALITCYLTWSPCLDCSQKLAALKRDHPGLTLRIFTSRLYFHWVKKFQEGL

SEQ ID NO: 70 - protein, 76 aa, Chinchilla lanigera
SPQKGHHAESRFIKRISSMDLDRSRSYQITCFLTWSPCPSCAQELASFKRAHPHLRFQIFVSRLYFHWKRSYQAGL

SEQ ID NO: 71 - protein, 74 aa, Heterocephalus glaber
KKGYHAESRFIKRICSMDLGQDQSYQVTCFLTWSPCPHCAQELVSFKRAHPHLRLQIFTARLFFHWKRSYQEGL

SEQ ID NO: 72 - protein, 74 aa, Octodon degus
KKGQHAEIRFIERIHSMALDQARSYQITCFLTWSPCPFCAQELASFKSTHPRVHLQIFVSRLYFHWKRSYQEGL

SEQ ID NO: 73 - protein, 75 aa, Urocitellus parryii
NKKGHHAEIRFIKKIRSLDLDQSQNYEVTCYLTWSPCPDCAQELVALTRSHPHVRLRLFTSRLYFHWFWSFQEGL

SEQ ID NO: 74 - protein, 72 aa, Aotus sp.
  source 1..72: note = Aotus nancymaae
NRHAEICFIDEIESMGLDKTQCYEVTCYLTWSPCPSCAQKLAAFTKAQVHLNLRIFASRLYYHWRSSYQKGL

SEQ ID NO: 75 - protein, 72 aa, Cebus capucinus
  source 1..72: note = Cebus capucinus imitator
NRHAEICFIDEIESMGLDKTQCYEVTCYLTWSPCPSCAQKLVAFAKAQDHLNLRIFASRLYYHWRRRYKEGL

SEQ ID NO: 76 - protein, 70 aa, Saimiri sp.
  source 1..70: note = Saimiri boliviensis boliviensis
HVEICFIDKIASMELDKTQCYDVTCYLTWSPCPSCAQKLAAFAKAQDHLNLRIFASRLYYHWRRSYQKGL

SEQ ID NO: 77 - protein, 75 aa, Homo sapiens
NKKKCHAEICFINEIKSMGLDETQCYQVTCYLTWSPCSSCAWELVDFIKAHDHLNLGIFASRLYYHWCKPQQKGL

SEQ ID NO: 78 - protein, 76 aa, Homo sapiens
ENKKKCHAEICFINEIKSMGLDETQCYQVTCYLTWSPCSSCAWELVDFIKAHDHLNLGIFASRLYYHWCKPQQKGL

SEQ ID NO: 79 - protein, 75 aa, Pan paniscus
NKKKCHAEICFINEIKSMGLDETQCYQVTCYLTWSPCSSCAWKLVDFIQAHDHLNLRIFASRLYYHWCKPQQEGL

SEQ ID NO: 80 - protein, 75 aa, Symphalangus syndactylus
NKKKRHAEIRFINKIKSMGLDETQCYQVTCYLTWSPCPSCAWELVDFIKAHDHLNLGIFASRLYYHWCRHQQEGL

SEQ ID NO: 81 - protein, 75 aa, Macaca mulatta
NKKKDHAEIRFINKIKSMGLDETQCYQVTCYLTWSPCPSCAGELVDFIKAHRHLNLRIFASRLYYHWRPNYQEGL

SEQ ID NO: 82 - protein, 75 aa, Theropithecus gelada
NKKKEHAEIRFINKIKSMGLDETQCYQVTCYLTWSPCPSCAGKLVDFIKAHHHLNLRIFASRLYYHWRPNYQEGL

SEQ ID NO: 83 - protein, 75 aa, Mandrillus leucophaeus
NKKKHHAEIHFINKIKSMGLDETQCYQVTCYLTWSPCPSCARELVDFIKAHRHLNLRIFASRLYYHWRPHYQEGL

SEQ ID NO: 84 - protein, 75 aa, Bos grunniens
NKKQRHAEIRFIDKINSLDLNPSQSYKIICYITWSPCPNCANELVNFITRNNHLKLEIFASRLYFHWIKPFKMGL

SEQ ID NO: 85 - protein, 75 aa, Bubalus bubalis
NKKQRHAEIRFIDKINSLDLNPSQSYKIICYITWSPCPNCASELVDFITRNDHLDLQIFASRLYFHWIKPFKRGL

SEQ ID NO: 86 - protein, 75 aa, Odocoileus virginianus
  source 1..75: note = Odocoileus virginianus texanus
NKKQRHAEIRFIDKINSLNLDRRQSYKIICYITWSPCPRCASELVDFITGNDHLNLQIFASRLYFHWKKPFQRGL

SEQ ID NO: 87 - protein, 75 aa, Sus scrofa
NKKKRHAEIRFIDKINSLNLDQNQCYRIICYVTWSPCHNCAKELVDFISNRHHLSLQLFASRLYFHWVRCYQRGL

SEQ ID NO: 88 - protein, 75 aa, Ceratotherium simum
  source 1..75: note = Ceratotherium simum simum
NKKKRHAEIRFIDKIKSLGLDRVQSYEITCYITWSPCPTCALELVAFTRDYPRLSLQIFASRLYFHWRRRSIQGL

SEQ ID NO: 89 - protein, 75 aa, Equus caballus
NKKKRHAEIRFIDKINSLGLDQDQSYEITCYVTWSPCATCACKLIKFTRKFPNLSLRIFVSRLYYHWFRQNQQGL

SEQ ID NO: 90 - protein, 74 aa, Enhydra lutris
  source 1..74: note = Enhydra lutris kenyoni
KKKRHAEIRFIDSIRALQLDQSQRFEITCYLTWSPCPTCAKELAMFVQDHPHISLRLFASRLYFHWRWKYQEGL

SEQ ID NO: 91 - protein, 74 aa, Leptonychotes weddellii
KKKRHAEIRFIDNIKALRLDTSQRFEITCYVTWSPCPTCAKELVAFVRDHRHISLRLFASRLYFHWLRENKKGL

SEQ ID NO: 92 - protein, 75 aa, Ursus arctos
  source 1..75: note = Ursus arctos horribilis
NKKKRHAEIRFIDKIRSLQRDSSQTFEITCYVTWSPCFTCAEELVAFVRDHPHVRLRLFASRLYFHWLRKYQEGL

SEQ ID NO: 93 - protein, 75 aa, Panthera leo
  source 1..75: note = Panthera leo bleyenberghi
NKKKRHAEICFIDKIKSLTRDTSQRFEIICYITWSPCPFCAEELVAFVKDNPHLSLRIFASRLYVHWRWKYQQGL

SEQ ID NO: 94 - protein, 75 aa, Panthera tigris; identical to SEQ ID NO: 93
  source 1..75: note = Panthera tigris sumatrae

SEQ ID NO: 95 - protein, 75 aa, Tupaia belangeri
NKKHRHAEVRFIAKIRSMSLDLDQKHQLTCYLTWSPCPSCAQELVTFMAESRHLNLQVFVSRLYFHWQRDFQQGL

SEQ ID NO: 96 - protein, 110 aa, Gorilla gorilla
GRSYNWLCYEVKIKRGRSNLLWNTGVFRGQMYSQPEHHAEMCFLSWFCGNQLPAYKCFQITWFVSWTPCPDCVAKLAEFLAEYPNVTLTISTARLYYYWERDYRRALCRL

SEQ ID NO: 97 - protein, 110 aa, Pan paniscus
GRSYTWLCYEVKIRRGHSNLLWDTGVFRGQMYSQPEHHAEMYFLSWFCGNQLPAYKCFQITWFVSWTPCPDCVAKLAEFLAEHPNVTLTISAARLYYYWERDYRRALCRL

SEQ ID NO: 98 - protein, 110 aa, Pan troglodytes
GRSYTWLCYEVKIRRGHSNLLWDTGVFRGQMYSQPEHHAEMCFLSWFCGNQLSAYKCFQITWFVSWTPCPDCVAKLAKFLAEHPNVTLTISAARLYYYWERDYRRALCRL

SEQ ID NO: 99 - protein, 98 aa, Gorilla gorilla
RNTVWLCYEVKTKGPSRPPLDAKIFRGQVYFEPQYHAEMCFLSWFCGNQLPAYKCFQITWFVSWTPCPDCVAKLAEFLAEHPNVTLTISAARLYYYWE

SEQ ID NO: 100 - protein, 108 aa, Pan troglodytes
RNTVWLCYEVKTKGPSRPRLDTKIFRGQVYFEPQYHAEMCFLSWFCGNQLPAYKCFQITWFVSWTPCPDCVAKLAEFLAEHPNVTLTISAARLYYYWERDYRRALCRL

SEQ ID NO: 101 - protein, 108 aa, Homo sapiens
RNTVWLCYEVKTKGPSRPRLDAKIFRGQVYSQPEHHAEMCFLSWFCGNQLPAYKCFQITWFVSWTPCPDCVAKLAEFLAEHPNVTLTISAARLYYYWERDYRRALCRL

SEQ ID NO: 102 - protein, 108 aa, Macaca sp.
  source 1..108: note = Macaca leonine
RNTVWLCYEVKTRGPSMPTWGTKIFRGQVCFEPQYHAEMCFLSRFCGNQLPAYKRFQITWFVSWTPCPDCVAKVAEFLAEHPNVTLTISAARLYYYWETDYRRALCRL

SEQ ID NO: 103 - protein, 108 aa, Macaca nemestrina; identical to SEQ ID NO: 102

SEQ ID NO: 104 - protein, 108 aa, Rhinopithecus roxellana
RNTVWLCYEVKTRGPSMPTWGAKIFRGQVYFEPQYHAEMCFLSWFCGNQLPAYKRFQITWFVSWTPCPDCVAKVAEFLAEHPNVTLTISAARLYYYWETDYRRALCRL

SEQ ID NO: 105 - protein, 101 aa, Mandrillus leucophaeus
RNTVWLCYKVKTRGPSMPTWGTKIFRGQVYFQPQYHAEMCFLSWFCGNQLPAYKRFQITWFVSWTPCPDCVVKVAEFLAEHPNVTLTISAARLYYYWETDY

SEQ ID NO: 106 - protein, 108 aa, Macaca mulatta
RNTVWLCYEVKTRGPSMPTWDTKIFRGQVYSKPEHHAEMCFLSRFCGNQLPAYKRFQITWFVSWTPCPDCVAKVAEFLAEHPNVTLTISAARLYYYWETDYRRALCRL

SEQ ID NO: 107 - protein, 108 aa, Theropithecus gelada
RNTVWLCYEVKTRGPSMPTWGTKIFRGQVYFQPQYHAEMCFLSRFCGNQLPAYKRFQITWFVSWNPCPDCVAKVIEFLAEHPNVTLTISAARLYYYWGRDWRRALRRL

SEQ ID NO: 108 - protein, 110 aa, Cercocebus atys
GRSYTWLCYEVKIRKDPSKLPWYTGVFRGQVYSKPEHHAEMCFLSRFCGNQLPAYKRFQITWFVSWNPCPDCVAKVIEFLAEHPNVTLTISAARLYYYWSRDWQRALCRL

SEQ ID NO: 109 - protein, 110 aa, Macaca fascicularis
GRSYTWLCYEVKIRKDPSKLPWDTGVFRGQVYSKPEHHAEMCFLSRFCGNQLPAYKRFQITWFVSWNPCPDCVAKVIEFLAEHPNVTLTISTARLYYYWGRDWQRALCRL

SEQ ID NO: 110 - protein, 110 aa, Macaca mulatta; identical to SEQ ID NO: 109

SEQ ID NO: 111 - protein, 110 aa, Macaca leonina
GRSYTWLCYEVKIRKDPSKLPWDTGVFRGQVYSKPEHHAEMCFLSRFCGNQLPAYKRFQITWFVSWNPCPDCVVKVIEFLAEHPNVTLTISTARLYYYWGRDWQRALCRL

SEQ ID NO: 112 - protein, 110 aa, Mandrillus leucophaeus
GRSYTWLCYEVKIRKDPSKLPWYTGVFRGQVYSKPEHHAEMCFLSRFCGNQLPAYKRFQITWFVSWNPCPDCVAKVIEFLAEHPNVTLTIFTARLYYYWGRDWQRALCRL

SEQ ID NO: 113 - protein, 110 aa, Macaca nemestrina
GRSYTWLCYEVKIRKDPSKLPWDTGVFRGQVYSKPEHHAEMCFLSRFCGNQLPAYKRFQITWFVSWNPCPDCVAKVTEFLAEHPNVTLTISTARLYYYWGRDWQRALCRL

SEQ ID NO: 114 - protein, 110 aa, Rhinopithecus bieti
GRSYTWLCYEVKIRKDPSKLPWDTGVFRGQVYSEPEHHAEMYFLSWFCGNQLPAYKRFQITWFVSWTPCPDCVAKVAEFLTEHPNVTLTISAARLYYYRGRDWRRALCRL

SEQ ID NO: 115 - protein, 110 aa, Rhinopithecus roxellana; identical to SEQ ID NO: 114

SEQ ID NO: 116 - protein, 110 aa, Chlorocebus sabaeus
GRSYTWLCYEVKIRKDPSKLPWDTGVFRGQMYSKPEHHAEMCFLSWFCGNQLPAHKRFQITWFVSWTPCPDCVAKVAEFLAEYPNVTLTISAARLYYYWETDYRRALCRL

SEQ ID NO: 117 - protein, 109 aa, Nomascus leucogenys
RSYTWLCYEVKIRKDPSKLPWDTGVFRGQMYFQPEYHAEMCFLSWFCGNQLPAYKRFQITWFVSWTPCPDCVAKVAVFLAEHPNVTLTISAARLYYYWEKDWQRALCRL

SEQ ID NO: 118 - protein, 110 aa, Cercocebus atys
GRSYTWLCYEVKIKKYPSKLLWDTGVFQGQVYFQPQYHAEMCFLSRFCGNQLPAYKRFQITWFVSWNPCPDCVAKVTEFLAEHPNVTLTISAARLYYYWEKDXRRALRRL

SEQ ID NO: 119 - protein, 110 aa, Papio anubis
GRSYTWLCYEVKIKEDPSKLLWDTGVFQGQVYFQPQYHAEMCFLSRFCGNQLPAYKRFQITWFVSWNPCPDCVAKVTEFLAEHPNVTLTISAARLYYYWGRDWRRALRRL

SEQ ID NO: 120 - protein, 122 aa, Chlorocebus aethiops
GRRYTWLCYEVKIKKDPSKLPWDTGVFPGQVRPKFQSNRRYEVYFQPQYHAEMYFLSWFCGNQLPAYKHFQITWFVSWNPCPDCVAKVTEFLAEHRNVTLTISAARLYYYWGKDWRRALCRL

SEQ ID NO: 121 - protein, 106 aa, Chlorocebus sabaeus
GRRYTWLCYEVKIKKDPSKLPWDTGVFPGQPQYHAEMYFLSWFCGNQLPAYKHFQITWFVSWNPCPDCVAKVTEFLAEHRNVTLTISAARLYYYWGKDWRRALCRL

SEQ ID NO: 122 - protein, 122 aa, Chlorocebus sabaeus
GRRYTWLCYEVKIKKDPSKLPWDTGVFPGQVRPKFQSNRRQKVYFQPQYHAEMYFLSWFCGNQLPAYKHFQITWFVSWNPCPDCVAKVTEFLAEHRNVTLTISAARLYYYWGKDWRRALCRL

SEQ ID NO: 123 - protein, 122 aa, Erythrocebus patas
GRRYTWLCYEVKIKKDPSKLPWDTGVFQGQVRPKFQSNRRYEVYFQPQYHAEMCFLSWFCGNQLPAYKHFQITWFVSWNPCPDCVAKVTEFLAEHPNVTLTISAARLYYYWGKDWRRALCRL

SEQ ID NO: 124 - protein, 131 aa, Macaca fascicularis
GRSYTWLCYEVKIRKDPSKLPWDTGVFRGQVRPKLQSNRRYELSNWECRKRVYFQPQYHAEMYFLSWFCGNQLPANKRFQITWFASWNPCPDCVAKVTEFLAEHPNVTLTISVARLYYYRGKDWRRALRRL

SEQ ID NO: 125 - protein, 110 aa, Macaca fascicularis
GRSYTWLCYEVKIRKDPSKLPWDTGVFRGQVYFQPQYHAEMYFLSWFCGNQLPANKRFQITWFASWNPCPDCVAKVTEFLAEHPNVTLTISVARLYYYRGKDWRRALRRL

SEQ ID NO: 126 - protein, 110 aa, Macaca nemestrina
GRSYTWLCYEVKIRKDPSKLPWDTGVFRDQVYFQPQYHAEMCFLSWFCGNQLPANKRFQITWFVSWNPCPDCVTKVTEFLAEHPNVTLTISVARLYYYRGKDWRRALRRL

SEQ ID NO: 127 - protein, 110 aa, Macaca leonina
GRSYTWLCYEVKIRKDPSKLPWYTGVFRGQVYFQPQYHAEMCFLSWFCGNQLPANKRFQITWFVSWNPCPDCVAKVTEFLAEHPNVTLTISVARLYYYRGKDWRRALRRL

SEQ ID NO: 128 - protein, 110 aa, Macaca mulatta
GRSYTWLCYEVKIRKDPSKLPWDTGVFRGQVYFQPQYHAEMCFLSWFCGNQLPAYKRFQITWFVSWNPCPDCVAKVTEFLAEHPNVTLTISVARLYYYRGKDWRRALCRL

SEQ ID NO: 129 - protein, 122 aa, Gorilla gorilla
GRSYTWLCYEVKIRRGSSNLLWNTGVFRGPVPPKLQSNHRQEVYFQFENHAEMCFLSWFCGNRLPANRRFQITWFVSWNPCLPCVVKVTKFLAEHPNVTLTISAARLYYYRDREWRRVLRRL

SEQ ID NO: 130 - protein, 122 aa, Pan paniscus
GRSYTWLCYEVKIKRGCSNLIWDTGVFRGPVLPKLQSNHRQEVYFQFENHAEMCFFSWFCGNRLPANRRFQITWFVSWNPCLPCVVKVTKFLAEHPNVTLTISAARLYYYQDREWRRVLRRL

SEQ ID NO: 131 - protein, 122 aa, Pan troglodytes; identical to SEQ ID NO: 130

SEQ ID NO: 132 - protein, 122 aa, Homo sapiens
GRSYTWLCYEVKIKRGRSNLLWDTGVFRGPVLPKRQSNHRQEVYFRFENHAEMCFLSWFCGNRLPANRRFQITWFVSWNPCLPCVVKVTKFLAEHPNVTLTISAARLYYYRDRDWRWVLLRL

SEQ ID NO: 133 - protein, 122 aa, Nomascus leucogenys
GRSYTWLCYEVKIRKDPSKLPWDKGVFRGQVLPKFQSNHRQEVYFQLENHAEMCFLSWFCGNQLPANRRFQITWFVSWNPCLPCVAKVTEFLAEHPNVTLTISAARLYYYRGRDWRRALRRL

SEQ ID NO: 134 - protein, 110 aa, Saimiri boliviensis
GKKYTWLCYEVKIKKDTSKLPWNTGVFRGQVNFNPEHHAEMYFLSWFRGKLLPACKRSQITWFVSWNPCLYCVAKVAEFLAEHPNVTLTVSTARLYCYWKKDWRRALRKL

SEQ ID NO: 135 - protein, 110 aa, Saimiri boliviensis; identical to SEQ ID NO: 134

SEQ ID NO: 136 - protein, 110 aa, Piliocolobus tephrosceles
GRRYTWLCYEVKIMKDHSKLPWYTGVFRGQVYFEPQNHAEMCFLSWFCGNQLPAYECCQITWFVSWTPCPDCVAKVTEFLAEHPNVTLTISAARLYYYRGRDWRRALRRL

SEQ ID NO: 137 - protein, 110 aa, Colobus sp.
  source 1..110: note = Colobus angolensis palliatus
GRRYTWLCYEVKISKDPSKLPWDTGIFRGQVYFEPQYHAEMCFLSWYCGNQLPAYKCFQITWFVSWTPCPDCVGKVAEFLAEHPNVTLTISAARLYYYWETDYRRALCRL

SEQ ID NO: 138 - protein, 121 aa, Pongo abelii
RNYTWLCYEVKIRKDPSKLAWDTGVFRGQVLPKLQSNHRREVYFEPQYHAEMCFLSWFCGNQLSAYERFQITWFVSWTPCPDCVAMLAEFLAEHPNVTLTVSAARLYYYWERDYRGALRRL

SEQ ID NO: 139 - RNA, 19 nt, Escherichia coli
  source 1..19: mol_type = other RNA; note = Bacteriophage MS2
acatgaggatcacccatgt

SEQ ID NO: 140 - RNA, 25 nt, Pseudomonas aeruginosa
  source 1..25: mol_type = other RNA; note = Bacteriophage PP7
ggagcagacgatatggcgtcgctcc

SEQ ID NO: 141 - RNA, 15 nt, Mycobacterium sp.
  source 1..15: mol_type = other RNA; note = Bacteriophage boxB
gccctgaagaagggc

SEQ ID NO: 142 - RNA, 48 nt, synthetic construct
  source 1..48: mol_type = other RNA
ttgtgtttctacttatagatggctaaaatctgagtttagaaaatgcaa

SEQ ID NO: 143 - RNA, 10 nt, synthetic construct
  source 1..10: mol_type = other RNA
aatttttgga

SEQ ID NO: 144 - RNA, 19 nt, synthetic construct
  source 1..19: mol_type = other RNA
ctgaatgcctgcgagcatc

SEQ ID NO: 145 - protein, 621 aa, synthetic construct
MELDPDDVFRDEDEDPENDFFQEKEASKEFVVYLIDASPKMFCSTCPSEEEDKQESHFHIAVSCIAQSLKAHIINRSNDEIAICFFNTREKKNLQDLNGVYVFNVPERDSIDRPTARLIKEFDLIEESFDKEIGSQTGIVSDSRENSLYSALWVAQALLRKGSLKTADKRMFLFTNEDDPFGSMRISVKEDMTRTTLQRAKDAQDLGISIELLPLSQPDKQFNITLFYKDLIGLNSDELTEFMPSVGQKLEDMKDQLKKRVLAKRIAKRITFVICDGLSIELNGYALLRPAIPGSITWLDSTTNLPVKVERSYICTDTGAIMQDPIQRIQPYKNQNIMFTVEELSQVKRISTGHLRLLGFKPLSCLKDYHNLKPSTFLYPSDKEVIGSTRAFIALHRSMIQLERFAVAFYGGTTPPRLVALVAQDEIESDGGQVEPPGINMIYLPYANDIRDIDELHSKPGVAAPRASDDQLKKASALMRRLELKDFSVCQFANPALQRHYAILQAIALDENELRETRDETLPDEEGMNRPAVVKAIEQFKQSIYGDDPDEESDSGAKEKSKKRKAGDADDGKYDYIELAKTGKLKDLTVVELKTYLTANNLLVSGKKEVLINRILTHIGK

SEQ ID NO: 146 - protein, 629 aa, synthetic construct
MSSESTTFIVDVSPSMMKNNNVSKSMAYLEYTLLNKSKKSRKTDWISCYLANCPVSENSQEIPNVFQIQSFLAPVTTTATIGFIKRLKQYCDQHSHDSSNEGLQSMIQCLLVVSLDIKQQFQARKILKQIVVFTDNLDDLDITDEEIDLLTEELSTRIILIDCGKDTQEERKKSNWLKLVEAIPNSRIYNMNELLVEITSPATSVVKPVRVFSGELRLGADILSTQTSNPSGSMQDENCLCIKVEAFPATKAVSGLNRKTAVEVEDSQKKERYVGVKSIIEYEIHNEGNKKNVSEDDQSGSSYIPVTISKDSVTKAYRYGADYVVLPSVLVDQTVYESFPGLDLRGFLNREALPRYFLTSESSFITADTRLGCQSDLMAFSALVDVMLENRKIAVARYVSKKDSEVNMCALCPVLIEHSNINSEKKFVKSLTLCRLPFAEDERVTDFPKLLDRTTTSGVPLKKETDGHQIDELMEQFVDSMDTDELPEIPLGNYYQPIGEVTTDTTLPLPSLNKDQEENKKDPLRIPTVFVYRQQQVLLEWIHQLMINDSREFEIPELPDSLKNKISPYTHKKFDSTKLVEVLGIKKVDKLKLDSELKTELEREKIPDLETLLKRGEQHSRGSPNNSNN

SEQ ID NO: 147 - protein, 70 aa, synthetic construct
GSVIDVSSQRVNVQRPLDALGNSLNSPVIIKLKGDREFRGVLKSFDLHMNLVLNDAEELEDGEVTRRLGT

SEQ ID NO: 148 - protein, 62 aa, synthetic construct
MKSIRCKNCNKLLFKADSFDHIEIRCPRCKRHIIMLNACEHPTEKHCGKREKITHSDETVRY

SEQ ID NO: 149 - RNA, 59 nt, synthetic construct
  source 1..59: mol_type = other RNA
gtttgagagctatgctgttttgggccaacatgaggatcacccatgtctgcagggccttt

SEQ ID NO: 150 - RNA, 76 nt, synthetic construct
  source 1..76: mol_type = other RNA
gtttgagagctatgctgttttgggccagcgaggagcaccagccctcgcgtgtgacgatgacgatcacgcatcgttt

SEQ ID NO: 151 - RNA, 79 nt, synthetic construct
  source 1..79: mol_type = other RNA
gtttgagagctatgctgttttgggccaggaatgaccaccaggcattccgatccgacgatggaccatcaggccatcgttt

SEQ ID NO: 152 - RNA, 44 nt, synthetic construct
  source 1..44: mol_type = other RNA
gtttgagagctatgctgttttggggccctgaagaagggcccttt

SEQ ID NO: 153 - RNA, 63 nt, synthetic construct
  source 1..63: mol_type = other RNA
gtttgagagctatgctgttttggggccctgaagaagggcccgggccctgaagaagggcccttt

SEQ ID NO: 154 - RNA, 69 nt, synthetic construct
  source 1..69: mol_type = other RNA
  modified_base 1..3: mod_base = OTHER; note = 2'-O-methyl
  modified_base 1..4: mod_base = OTHER; note = phosphorothioate linkage
  modified_base 67..69: mod_base = OTHER; note = 2'-O-methyl
  modified_base 66..69: mod_base = OTHER; note = phosphorothioate linkage
actccacccagtttgagagctatgctgttttgggccaacatgaggatcacccatgtctgcagggccttt

SEQ ID NO: 155 - RNA, 86 nt, synthetic construct
  source 1..86: mol_type = other RNA
  modified_base 1..3: mod_base = OTHER; note = 2'-O-methyl
  modified_base 1..4: mod_base = OTHER; note = phosphorothioate linkage
  modified_base 83..86: mod_base = OTHER; note = phosphorothioate linkage
  modified_base 84..86: mod_base = OTHER; note = 2'-O-methyl
actccacccagtttgagagctatgctgttttgggccagcgaggagcaccagccctcgcgtgtgacgatgacgatcacgcatcgttt

SEQ ID NO: 156 - RNA, 89 nt, synthetic construct
  source 1..89: mol_type = other RNA
  modified_base 1..3: mod_base = OTHER; note = 2'-O-methyl
  modified_base 1..4: mod_base = OTHER; note = phosphorothioate linkage
  modified_base 87..89: mod_base = OTHER; note = 2'-O-methyl
  modified_base 86..89: mod_base = OTHER; note = phosphorothioate linkage
actccacccagtttgagagctatgctgttttgggccaggaatgaccaccaggcattccgatccgacgatggaccatcaggccatcgttt

SEQ ID NO: 157 - RNA, 64 nt, synthetic construct
  source 1..64: mol_type = other RNA
  modified_base 1..3: mod_base = OTHER; note = 2'-O-methyl
  modified_base 1..4: mod_base = OTHER; note = phosphorothioate linkage
  modified_base 62..64: mod_base = OTHER; note = 2'-O-methyl
  modified_base 61..64: mod_base = OTHER; note = phosphorothioate linkage
cttgaccaatagccttgacagtttgagagctatgctgttttggggccctgaagaagggcccttt

SEQ ID NO: 158 - RNA, 83 nt, synthetic construct
  source 1..83: mol_type = other RNA
  modified_base 1..3: mod_base = OTHER; note = 2'-O-methyl
  modified_base 1..4: mod_base = OTHER; note = phosphorothioate linkage
  modified_base 81..83: mod_base = OTHER; note = 2'-O-methyl
  modified_base 80..83: mod_base = OTHER; note = phosphorothioate linkage
cttgaccaatagccttgacagtttgagagctatgctgttttggggccctgaagaagggcccgggccctgaagaagggcccttt

SEQ ID NO: 159 - protein, 152 aa, Rhizobium sp.
  source 1..152: note = Agrobacterium fabrum
MAERTHFMELALVEARSAGERDEVPIGAVLVLDGRVIARSGNRTRELNDVTAHAEIAVIRMACEALGQERLPGADLYVTLEPCTMCAAAISFARIRRLYYGAQDPKGGAVESGVRFFSQPTCHHAPDVYSGLAESESAEILRQFFREKRLDD

SEQ ID NO: 160 - protein, 1307 aa, Arabidopsis thaliana
MFNTYTNSLQWPIRSRNQQDYCSLLPERSESYKLSKAYTSSRCYCVSSRSSCCCCCSTPSSSSFVKPKVLINPGFVLYGVRQSTLIQWPSFQRRLLVGGGRLMGCEVYSSCDGIRRKNRSFKLRCLEESDECCGGRSCSDDVEAMISFLSEELIDEERKWNLVSRVKEKKKVGNVRKVSVEGSNSYGNGRVSQRVKKPEGFGRRKEIKEDVKLNERYDCEHCGRRKKSSELESESRRGSKLVTGEYIGKSYRGDEEREVRPRRRKSSSCSSYYSLASSGEFESDTEDQEEDVEIYRENVRSSEKKVVDQSAKRLKSRKEASQMHSRKKRDESSTGVDSRYQKQIFEEGENSNQAVTLNQRRRKKFSQTENRVSESTGNYEEDMEIHEVHVNDAETSSQNQKLFNEREDYRVHSIRNDSGNENIESSQHQLKERLETRYSSEDRVSEMRRRTKYSSSQEEGINVLQNFPEVTNNQQPLVEERISKQAGTRRTTEHISESSEIHDIDIRNTYVSQREDQIRNQEVHAGLVSGLQSERKQQDYHIEHNPLQTTQSDRTSVSVSHTSDAVRYTEIQRKSEKRLIGQGSTTAVQSDSKVEKNGAQKEDSRLDHANSKKDGQTTLGLQSYQSKLSEEASSSQSSLMASRTKLQLVDLVSEEMQGSETTLIPPSSQLVSRRSGQSYRTGGVSIQEISHGTSESGYTTAFEHPRAGASVNSQSAGELMGFTSHEDAMGSAHRLEQASEKYVGEFVKKAKHGVINPETEEQRAESNQLKRRDSRRSSGGSGAKGPSDEMWVTDSAQGTPHPGATEGNAAVGNAIFKRNGRSLWNVIADIARLRWGSRAGSPDSSAKPAGRSSPNESVSSATWFSGREHDGSSDDNTKGDKVLPQEAPSLHQVEVGQTSPRSQSEYPGTTKLKQRSERHEGVVSSPSSTILEGGSVSNRMSSTSGNQIVGVDEEEGGNFEFRLPETALTEVPMKLPSRNLIRSPPIKESSESSLTEASSDQNFTVGEGRRYPRMDAGQNPLLFPGRNLRSPAVMEPPVPRPRMVSGSSSLREQVEQQQPLSAKSQEETGSVSADSALIQRKLQRNKQVVRDSFEEWEEAYKVEAERRTVDEIFMREALVEAKKAADTWEVPVGAVLVHDGKIIARGYNLVEELRDSTAHAEMICIREGSKALRSWRLADTTLYVTLEPCPMCAGAILQARVNTLVWGAPNKLLGADGSWIRLFPGGEGNGSEASEKPPPPVHPFHPKMTIRRGVLESECAQTMQQFFQLRRKKKDKNSDPPTPTDHHHHHLPKLLNKMHQVLPFFCL

SEQ ID NO: 161 - protein, 151 aa, Aquifex sp.
  source 1..151: note = Aquifex aeolicus
MGKEYFLKVALREAKRAFEKGEVPVGAIIVKEGEIISKAHNSVEELKDPTAHAEMLAIKEACRRLNTKYLEGCELYVTLEPCIMCSYALVLSRIEKVIFSALDKKHGGVVSVFNILDEPTLNHRVKWEYYPLEEASELLSEFFKKLRNNII

SEQ ID NO: 162 - protein, 171 aa, Streptococcus pyogenes
MPYSLEEQTYFMQEALKEAEKSLQKAEIPIGCVIVKDGEIIGRGHNAREESNQAIMHAEMMAINEANAHEGNWRLLDTTLFVTIEPCVMCSGAIGLARIPHVIYGASNQKFGGADSLYQILTDERLNHRVQVERGLLAADCANIMQTFFRQGRERKKIAKHLIKEQSDPFD

SEQ ID NO: 163 - protein, 161 aa, Bacillus subtilis
MTQDELYMKEAIKEAKKAEEKGEVPIGAVLVINGEIIARAHNLRETEQRSIAHAEMLVIDEACKALGTWRLEGATLYVTLEPCPMCAGAVVLSRVEKVVFGAFDPKGGCSGTLMNLLQEERFNHQAEVVSGVLEEECGGMLSAFFRELRKKKKAARKNLSE

SEQ ID NO: 164 - protein, 173 aa, Haemophilus influenzae
MDAAKVRSEFDEKMMRYALELADKAEALGEIPVGAVLVDDARNIIGEGWNLSIVQSDPTAHAEIIALRNGAKNIQNYRLLNSTLYVTLEPCTMCAGAILHSRIKRLVFGASDYKTGAIGSRFHFFDDYKMNHTLEVTSGVLAEECSQKLSTFFQKRREEKKIEKALLKSLSDK

SEQ ID NO: 165 - protein, 161 aa, Buchnera aphidicola
MKYEKDKNWMKIALKYAYYAKEKGEIPIGAILVFKERIIGIGWNSSISKNDPTAHAEIIALRGAGKKIKNYRLLNTTLYVTLQPCIMCCGAIIQSRIKRLVFGANCNSSDHRFSLKNLFCDPQKDYKLDIKKNVMQRECSDILINFFQKKRKNKIHICKKI

SEQ ID NO: 166 - protein, 167 aa, Escherichia coli
MSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 167 - protein, 171 aa, Streptococcus pyogenes
MPYSLEEQTYFMQEALKEAEKSLQKAEIPIGCVIVKDGEIIGRGHNAREESNQAIMHAEMMAINEANAHEGNWRLLDTTLFVTIEPCVMCSGAIGLARIPHVIYGASNQKFGGVDSLYQILTDERLNHRVQVERGLLAADCANIMQTFFRQGRERKKIAKHLIKEQSDPFD

SEQ ID NO: 168 - protein, 145 aa, Rickettsia bellii
MREALKQAEIAFSKNEVPVGAVIVDRENQKIISKSYNNTEEKNNALYHAEIIAINEACRIISSKNLSDYDIYVTLEPCAMCAAAIAHSRLKRLFYGASDSKHGAVESNLRYFNSKACFHRPEIYSGIFAEDSALLMKGFFKKIRD

SEQ ID NO: 169 - protein, 144 aa, Rickettsia felis
MEQALKQAGIAFDKNEVPVGAVIVDRLNQKIIVSSHNNTEEKNNALYHAEIIAINEACNLISSKNLNDYDIYVTLEPCAMCAAAIAHSRLKRLFYGASDSKHGAVESNLRYFNSSVCFYRPEIYSGILAEDSRLLMKEFFKRIR

SEQ ID NO: 170 - protein, 172 aa, Salmonella sp.
  source 1..172: note = Salmonella typhimurium
MSDVELDHEYWMRHALTLAKRAWDEREVPVGAVLVHNHRVIGEGWNRPIGRHDPTAHAEIMALRQGGLVLQNYRLLDTTLYVTLEPCVMCAGAMVHSRIGRVVFGARDAKTGAAGSLIDVLHHPGMNHRVEIIEGVLRDECATLLSDFFRMRRQEIKALKKADRAEGAGPAV

SEQ ID NO: 171 - protein, 167 aa, Escherichia coli
MSEVEFSHEYWMRHAMTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 172 - protein, 151 aa, Buchnera sp.
  source 1..151: note = Buchnera aphidicola subsp. Baizongia pistaciae (strain Bp)
MKSNRDSYWMKIALKYAYYAEENGEVPIGAILVFQEKIIGTGWNSVISQNDSTAHAEIIALREAGRNIKNYRLVNTTLYVTLQPCMMCCGAIINSRIKRLVFGASYKDLKKNPFLKKIFINLEKNKLKIKKHIMRNECAKILSNFFKNKRF

SEQ ID NO: 173 - protein, 159 aa, Streptococcus pyogenes
MPYSLEEQTYFMQEALKEAEKSLQKAEIPIGCVIVKDGEIIGRGHNAREESNQAIMHAEMMAINEANAHEGNWRLLDTTLFVTIEPCVMCSGAIGLARIPHVIYGASNQKFGGADSLYQILTDERLNHRVQVERGLLAADCANIMQTFFRQGRERKKNS

SEQ ID NO: 174 - protein, 148 aa, Rickettsia prowazekii
MEQALKQARLAFDKNEVPVGVVIVCRLNQKIIVSSHNNIEEKKNPLCHAEIIAINTACNLISSKNLNDYDIYVTLEPCAMCASAISHSRLKRLFYGASDSKHGAVESNLRYFNSNSCFYRPEIYSGILSEHSRFLMQEFFQRIRSAID

SEQ ID NO: 175 - protein, 148 aa, Rickettsia typhi
MEQALKQARLAFDKNEVPVGVVIVYRLNQKIIVSSHNNIEEKNNALCHAEIIAINEACNLISSKNLNDYDIYVTLEPCAMCASAISHSRLKRLFYGASDSKQGAVESNLRYFNSSACFHRPEIYSGILSEHSRFLMKEFFQKMRSTID

SEQ ID NO: 176 - protein, 162 aa, Buchnera sp.
  source 1..162: note = Buchnera aphidicola subsp. Schizaphis graminum (strain Sg)
MHDSDKYFMKCAIFLAKISEMIGEVPVGAVLVFNNTIIGKGLNSSILNHDPTAHAEIKALRNGAKFLKNYRLLHTTLYVTLEPCIMCYGAIIHSRISRLVFGAKYKNLQKYICCKNHFFINKNFRKISITQEVLESECSNLLSSFFKRKRKIATKYFNNNII

SEQ ID NO: 177 - protein, 153 aa, Rickettsia conorii
MEQALKQAKIAFDKNEVPVGAVVVDRLHQKIIASTHNNTEEKNNALYHAEIIAINEACNLISSKNLNDYDIYVTLEPCAMCAAAIAHSRLKRLFYGASDSKHGVVESNLRYFNSSACFHRPEIYSGILAEDSGLLMKEFFKRIRTVISSHRMT

SEQ ID NO: 178 - protein, 156 aa, Staphylococcus aureus
MTNDIYFMTLAIEEAKKAAQLGEVPIGAIITKDDEVIARAHNLRETLQQPTAHAEHIAIERAAKVLGSWRLEGCTLYVTLEPCVMCAGTIVMSRIPRVVYGADDPKGGCSGSLMNLLQQSNFNHRAIVDKGVLKEACSTLLTTFFKNLRANKKSTN

SEQ ID NO: 179 - protein, 420 aa, Arabidopsis thaliana
MEEDWGKTVSEKVISAYMSLPKKGKPQGREVTVLSAFLVSSPSQDPKVIALGTGTKCVSGSLLSPRGDIVNDSHAEVVARRALIRFFYSEIQRMQLTSGKSNEAKRQRIDSETSSILESADSSCPGEVKYKLKSGCLLHLYISQLPCGYASTSSPLYALKKIPSTQVDDSLLVQASDICSSRHSDVPEIGSNSNKGNGSQVADMVQRKPGRGETTLSVSCSDKIARWNVLGVQGALLYQVLQPVYISTITVGQSLHSPDNFSLADHLRRSLYERILPLSDELLTSFRLNKPLFFVAPVPPSEFQHSETAQATLTCGYSLCWNYSGLHEVILGTTGRKQGTSAKGALYPSTQSSICKQRLLELFLKETHGHKRESSKSKKSYRELKNKATEYYLMSKIFKGKYPFNNWLRKPLNCEDFLIN

SEQ ID NO: 180 - protein, 388 aa, Schizosaccharomyces pombe Tf1 virus
MEEFTLDRNSNVGNLIALAVLNKFDELARHGKPIIRANGVREWTTLAGVVIQKKMENEFICVCLATGVKCTPAGIIKNEQLGSVLHDCHAEILALRCFNRLLLEHCILIKESKKDTWLLEVADNGKFTLNSNLLIHLYVSECPCGDASMELLASRLENNKPWNLTVDSEKLMRGRADFGLLGIVRTKPGRPDAPVSWSKSCTDKLAAKQYLSILNSQTSLICEPIYLSCVVLYKKVIVKSAIDRAFGPFGRCAPLAEFGEKDNPYYFHPFTVLETDENFLYSRPLNQAEKTATSTNVLIWIGDKMQCTQVIHNGIKAGTKAKDVEKSQTLICRKSMMNLLHQLSQSLTNEKNYYEWKKLNIKRCQQKQILRNILKNWIPNGGNEFQWI

SEQ ID NO: 181 - protein, 400 aa, Saccharomyces cerevisiae
MVSCQGTRPCIVNLLTMPSEDKLGEEISTRVINEYSKLKSACRPIIRPSGIREWTILAGVAAINRDGGANKIEILSIATGVKALPDSELQRSEGKILHDCHAEILALRGANTVLLNRIQNYNPSSGDKFIQHNDEIPARFNLKENWELALYISRLPCGDASMSFLNDNCKNDDFIKIEDSDEFQYVDRSVKTILRGRLNFNRRNVVRTKPGRYDSNITLSKSCSDKLLMKQRSSVLNCLNYELFEKPVFLKYIVIPNLEDETKHHLEQSFHTRLPNLDNEIKFLNCLKPFYDDKLDEEDVPGLMCSVKLFMDDFSTEEAILNGVRNGFYTKSSKPLRKHCQSQVSRFAQWELFKKIRPEYEGISYLEFKSRQKKRSQLIIAIKNILSPDGWIPTRTDDVK

SEQ ID NO: 182 - protein, 502 aa, Macaca fascicularis
MWTADEIALLCYEHYGIRLPKKGKPEPNHEWTLLAAVVKIQSPADQDCDTPDKPAQVTKEVVSMGTGTKCIGQSKMRKSGDILNDSHAEVIARRNFQRYLLHQLQLAATLKEDSIFVPGTQKGLWKLRRDLFFVFFSSHTPCGDASIIPMLEFEDQPCCPVIRDWASSSSVEASSNLEAPGNERKCEDLDSPVTKKMRLEPMTAAREVTNGATHHQSFGKQESGPISPGINSCNLTVEGLAAVTRIAPGSAKVIDVYRTGAKCVPGEAGDSRKPGAAFHQVGLLRVKPGRGDRTRSMSCSDKMARWNVLGCQGALLMHFLEEPIYLSAVVIGKCPYSQEAMQRALTGRRQNVSALPKGFGVQELKILQSDLLFEQSRCAVQAKRADSPGRLVPCGAAISWSAVPEQPLDVTANGFPQGTTKKTIGSLQARSQISKVELLRSFQKLLSRIARDKWPDSLRVQKLDTYQDYKEAASSYQEAWSTLRKQAFGSWIRNPPDYHQFK

SEQ ID NO: 183 - protein, 503 aa, Gallus gallus
MWTADEIAELCYEHYRSRLPKQGKPDPSREWTSLAAVVKVESAANEAGSAVLGTLQVAKEVVALGTGTKCIGLNKMRKTGDVLNDSHAEVVAKRSFQRYLLHQMRLATSYQQCSIFIPGTETGKWKLKPNIIFIFFCSHTPCGDASIIPIRETENHLSKSVDGHDIAGQSVLCSSSNCDHRGPEDKRKSEKMASSHMIKRMKNADGGFFSTITEDMAVQQVFAKPEGNVNPECCESSEEMQAANKETNAGKLKAVGVYRTGAKFVPGELSDTLIPGIEYHCVGLLRVKPGRGDRTCSMSCSDKLARWNVLGCQGALLMHFLQYPVYLSAVIVGKCPYSQEAMQRAVIERCRHISLLPDGFLTQEVQLLQSDLQFEHSRQAIQEGQTSSKRKLVPCSAAISWSAVPEGPLDVTSDGFRQGTTKKGIGSPQSRSKICKVELFHEFQKLVTSISKENLPDTLRMKTLETYWDYKEAALNYQEAWKALRSQALLGWIKNAQEYLLFM

SEQ ID NO: 184 - protein, 502 aa, Homo sapiens
MWTADEIAQLCYEHYGIRLPKKGKPEPNHEWTLLAAVVKIQSPADKACDTPDKPVQVTKEVVSMGTGTKCIGQSKMRKNGDILNDSHAEVIARRSFQRYLLHQLQLAATLKEDSIFVPGTQKGVWKLRRDLIFVFFSSHTPCGDASIIPMLEFEDQPCCPVFRNWAHNSSVEASSNLEAPGNERKCEDPDSPVTKKMRLEPGTAAREVTNGAAHHQSFGKQKSGPISPGIHSCDLTVEGLATVTRIAPGSAKVIDVYRTGAKCVPGEAGDSGKPGAAFHQVGLLRVKPGRGDRTRSMSCSDKMARWNVLGCQGALLMHLLEEPIYLSAVVIGKCPYSQEAMQRALIGRCQNVSALPKGFGVQELKILQSDLLFEQSRSAVQAKRADSPGRLVPCGAAISWSAVPEQPLDVTANGFPQGTTKKTIGSLQARSQISKVELFRSFQKLLSRIARDKWPHSLRVQKLDTYQEYKEAASSYQEAWSTLRKQVFGSWIRNPPDYHQFK

SEQ ID NO: 185 - protein, 499 aa, Mus musculus
MWTADEIAQLCYAHYNVRLPKQGKPEPNREWTLLAAVVKIQASANQACDIPEKEVQVTKEVVSMGTGTKCIGQSKMRESGDILNDSHAEIIARRSFQRYLLHQLHLAAVLKEDSIFVPGTQRGLWRLRPDLSFVFFSSHTPCGDASIIPMLEFEEQPCCPVIRSWANNSPVQETENLEDSKDKRNCEDPASPVAKKMRLGTPARSLSNCVAHHGTQESGPVKPDVSSSDLTKEEPDAANGIASGSFRVVDVYRTGAKCVPGETGDLREPGAAYHQVGLLRVKPGRGDRTCSMSCSDKMARWNVLGCQGALLMHFLEKPIYLSAVVIGKCPYSQEAMRRALTGRCEETLVLPRGFGVQELEIQQSGLLFEQSRCAVHRKRGDSPGRLVPCGAAISWSAVPQQPLDVTANGFPQGTTKKEIGSPRARSRISKVELFRSFQKLLSSIADDEQPDSIRVTKKLDTYQEYKDAASAYQEAWGALRRIQPFASWIRNPPDYHQFK

SEQ ID NO: 186 - protein, 394 aa, Drosophila melanogaster
MCDNKKPTVKEIAELCLKKFESLPKTGKPTANQWTILAGIVEFNRNTEACQLVSLGCGTKCIGESKLCPNGLILNDSHAEVLARRGFLRFLYQELKQDRIFHWNSTLSTYDMDEHVEFHFLSTQTPCGDACILEEEQPAARAKRQRLDEDSEMVYTGAKLISDLSDDPMLQTPGALRTKPGRGERTLSMSCSDKIARWNVIGVQGALLDVLISKPIYFSSLNFCCDDAQLESLERAIFKRFDCRTFKHTRFQPQRPQINIDPGIRFEFSQRSDWQPSPNGLIWSQVPEELRPYEISVNGKRQGVTKKKMKTSQAALAISKYKLFLTFLELVKFNPKLSEMFDQQLSDPERIAYASCKDLARDYQFAWREIKEKYFLQWTKKPHELLDFNPMSNK

SEQ ID NO: 187 - protein, 472 aa, Xenopus tropicalis
MQAKGLWSADEIAALSYGHYTTQLPKQGLPDPSREWTLMAAVIQIESVEDTKVIKKVVAMGTGTKCIGQAKLRKTGDVLQDSHAEIIAKRSFQRYLLHQLSLAVSDTKDCLFIPGTEKGKWMLRPEISFVFFTSHTPCGDASIIPVISHEDELGHPLPSEVTEKDHSSNNVCESVNTTYKRKVRSEEDIGFISKKMKHSIDEILTRPENYEEENRHDFPSTCQKALDVHRTGAKCVAGELQDSYSPGVNYHTVGVLRIKPGRGDRTMSMSCSDKMARWNVLGCQGALLMHFLQQPIYLSAVVVGKCPFSQDAMERALYNRCHKVLSLPCAFRLNRVQIIQSDLEFQHGRHALTKKDATRKLVPCGAAVSWSAVPHHPLDVTANGYRQGTTRKAIGSPQCRSRICKAEIFNTFRELVQRLSEKQRSESLSSQGLKTYWDYKAAAITYQEAWNCLRQQAFTSWIQTPRDFLMFS

SEQ ID NO: 188 - protein, 545 aa, Dictyostelium discoideum Skipper virus
MSWKLDKQFSDKICNFSHDFFNKKLIKKGKPISGEWTVLATLVLVVENTSSYEIKQVLSLGTGNRCLGKSSLSNQGDVLNDSHAEIICKRSFQKFCYNEILNLLQSKYYNSILFNIEYHDSNNNNKDNDNNGSLPTISIKKGHSLHFYVNQTPCGDCSIFPFKKETQPENFIEKEKLEKDGKDKIENHEKKEQKDIIKQVDKDKDEENYEDEESKRKLKKVKDDNNNNNNNNNNNNNNNNNNNNNNNNNNNNNNNNNNNNNINNNNNQYDDIQRTGAKTVFGEPEDKKLIGVDYHQIGVLRVKPGRGDPTVSMSCSDKIARWNVLGIQGSLLSHFIKEQIFLSSITIGDLFNHSSIYRGLIGRLLPNPTTTTTETSSSSSSSSSSSNTIPNFKLNSDLEIFSTNIQFQFSKLLLESDQQNNNKSTSSGLAISFCYPNQHEVTIAINGKKMGTNQKNFNAISQRSSICKFNLFKLFHQLVLIIKNKNSNEENEKNNQIVLIDSLFNYYECKHLSKKYYQEYEKLKEFKFKNWLTNSSDLENFVLDN

SEQ ID NO: 189 - protein, 389 aa, Schizosaccharomyces pombe Tf1 virus
MAGDSVKSAIIGIAGGPFSGKTQLCEQLLERLKSSAPSTFSKLIHLTSFLYPNSVDRYALSSYDIEAFKKVLSLISQGAEKICLPDGSCIKLPVDQNRIILIEGYYLLLPELLPYYTSKIFVYEDADTRLERCVLQRVKAEKGDLTKVLNDFVTLSKPAYDSSIHPTRENADIILPQKENIDTALLFVSQHLQDILAEMNKTSSSNTVKYDTQHETYMKLAHEILNLGPYFVIQPRSPGSCVFVYKGEVIGRGFNETNCSLSGIRHAELIAIEKILEHYPASVFKETTLYVTVEPCLMCAAALKQLHIKAVYFGCGNDRFGGCGSVFSINKDQSIDPSYPVYPGLFYSEAVMLMREFYVQENVKAPVPQSKKQRVLKREVKSLDLSRFK

SEQ ID NO: 190 - protein, 250 aa, Saccharomyces cerevisiae
MQHIKHMRTAVRLARYALDHDETPVACIFVHTPTGQVMAYGMNDTNKSLTGVAHAEFMGIDQIKAMLGSRGVVDVFKDITLYVTVEPCIMCASALKQLDIGKVVFGCGNERFGGNGTVLSVNHDTCTLVPKNNSAAGYESIPGILRKEAIMLLRYFYVRQNERAPKPRSKSDRVLDKNTFPPMEWSKYLNEEAFIETFGDDYRTCFANKVDLSSNSVDWDLIDSHQDNIIQELEEQCKMFKFNVHKKSKV

SEQ ID NO: 191 - protein, 170 aa, Xenopus tropicalis
MTEEIQNWMHKAFQMAQDALNNGEVPVGCLMVYDNQVVGKGRNEVNETKNATRHAEMVAIDQVLDWCEKNSKKSRDVFENIVLYVTVEPCIMCAGALRLLKIPLVVYGCRNERFGGCGSVLNVAGDNIPDTGTEFKYIGGYQAEKAVELLKTFYKQENPNAPRSKVRKKE

SEQ ID NO: 192 - protein, 191 aa, Bos taurus
MEAKAGPTAATDGAYSVSAEETEKWMEQAMQMAKDALDNTEVPVGCLMVYNNEVVGKGRNEVNQTKNATRHAEMVAIDQALDWCRRRGRSPSEVFEHTVLYVTVEPCIMCAAALRLMRIPLVVYGCQNERFGGCGSVLDIASADLPSTGKPFQCTPGYRAEEAVEMLKTFYKQENPNAPKSKVRKKECHKS

SEQ ID NO: 193 - protein, 214 aa, Danio rerio
MQEVGVDPEKNDFLQPSDSEVQTWMAKAFDMAVEALENGEVPVGCLMVYNNEIIGKGRNEVNETKNATRHAEMVALDQVLDWCRLREKDCKEVCEQTVLYVTVEPCIMCAAALRLLRIPFVVYGCKNERFGGCGSVLDVSSDHLPHTGTSFKCIAGYRAEEAVEMLKTFYKQENPNAPKPKVRKDSINPQDGAAVIQVMRGPPDEETETIAHLS

SEQ ID NO: 194 - protein, 182 aa, Arabidopsis thaliana
MEEDHCEDSHNYMGFALHQAKLALEALEVPVGCVFLEDGKVIASGRNRTNETRNATRHAEMEAIDQLVGQWQKDGLSPSQVAEKFSKCVLYVTCEPCIMCASALSFLGIKEVYYGCPNDKFGGCGSILSLHLGSEEAQRGKGYKCRGGIMAEEAVSLFKCFYEQGNPNAPKPHRPVVQRERT

SEQ ID NO: 195 - protein, 191 aa, Mus musculus
MEEKVESTTTPDGPCVVSVQETEKWMEEAMRMAKEALENIEVPVGCLMVYNNEVVGKGRNEVNQTKNATRHAEMVAIDQVLDWCHQHGQSPSTVFEHTVLYVTVEPCIMCAAALRLMKIPLVVYGCQNERFGGCGSVLNIASADLPNTGRPFQCIPGYRAEEAVELLKTFYKQENPNAPKSKVRKKDCQKS

SEQ ID NO: 196 - protein, 191 aa, Homo sapiens
MEAKAAPKPAASGACSVSAEETEKWMEEAMHMAKEALENTEVPVGCLMVYNNEVVGKGRNEVNQTKNATRHAEMVAIDQVLDWCRQSGKSPSEVFEHTVLYVTVEPCIMCAAALRLMKIPLVVYGCQNERFGGCGSVLNIASADLPNTGRPFQCIPGYRAEEAVEMLKTFYKQENPNAPKSKVRKKECQKS

SEQ ID NO: 197 - protein, 400 aa, Arabidopsis thaliana
MDSDAWEIIHIPEKPSLSPDHQPTVKVYASLIKPRFANTIVRHLCKIAPLEDLRHVKRVKKKILPDCGETQLTVILCLAPEHNDQLSDMPPDVQRLVDPYELSPFITQVCKYAAVSKEEWEEQSKIWPTSFHPPTYNIDGIGGFSEEETQSICKFMRVVIDMAVSGHTPLVNAAVIVDPSVRRIIASETDQVYASSAPRDMTSAETRPFEETGEICLNDTLEKQNGSLSALSCLNPWQWSLQPHDTENCSQWHPLRHASMVAIESSSARDRNLFPNPSKIFDQDHVPPSNTDSPAKKQKTSSQSPDVQNDSREETVRDPSMERPYLCTGYDIFLLLEPCTMCAMALVHQRIKRIFYAFPNTTAGGLGSVHRLQGEKSLNHHYAVFRVLLPDDALRQMTTV

SEQ ID NO: 198 - protein, 349 aa, Mus musculus
MEPTSGFAEQPGPVKAESEEQEPAQWQALPVLSEQQSGAVELILAYAAPVLDKRQTSRLLREVSAVYPLPAQPHLKRVRPSRSAGGAQSSDLLLCLAGPSAGPRSLAELLPRPAVDPRGLGTPFLVPVPARPPLTRSQFEEARAHWPTSFHEDKQVTSALAGQLFSTQERAAMQTHMERAVCAAQRAAAQGLRAVGAVVVDPASDRVLATGHDCSSVASPLLHAVMVCIDLVAQGQGRGSCDLRSHPACSFTQATATQGARAGSVRKLDEDSLPYVCTGYDLYVTREPCVMCAMALVHARIQRVFYGAPSPDGALGTLFRVHARPDLNHRFQVFRGILEDQCRQLDPDP

SEQ ID NO: 199 - protein, 336 aa, Danio rerio
MEPQAKRKKEMDDYDDTWEVLPVLSDEQSQDPELLPAYAAPILERRETSRLVKELSLIHPLPNLQHIKRVRPCKHKDSPHPLEVIVCLVSDVQCTDPKKVTLSHLLHTQCFNSNGLGDPFIVQIPANPPLTRPQFEKASKHWPTSFHEDKLVTFALKGQLFTAHQKTKMREYMCVAVKAAKSGRELGMDAVGAVIVDPKTEQIIAVAHDCKRGSHPLHHAVMVCIDLVACGQDGGAYNYEKYPACRFSCSNSVCDGKETGLPYICTGYDLYVTREPCVMCAMALVHSRISRVFYGASTADGAFGSRYKIHCQKDLNHRFEVFKGVMVNACEDLCKE

SEQ ID NO: 200 - protein, 315 aa, Schizosaccharomyces pombe Tf1 virus
MVKTNISKNSPKEATVPELDWPFKLIKSHLETRKLETENVWIACFEPKYASKVTQYVKQIRSKQKESLLHCNRLRRIQDENGSLELQIIICPEKSMTANEIGKDFEDLGIVSKMIFLYAVPAFPPLTDEQFHEWNSVWPVSYRKHVQRQDVFTVHELKRIESILEDLINAAGASHKHGEIGCAAAIYDPTTDTVLAVSVDERSKLKNPINHCVMNAINLVAKRELSRRQNRTDGSKDRYLCKDLTVVMTHEPCVMCSMGLLHSRIRRLIYCKKQPLTGGIESLYGIHWRAELNHRYLAYSGWNKPVPSIKENIHV

SEQ ID NO: 201 - protein, 322 aa, Saccharomyces cerevisiae
MVKKVNNPLKIDYQNGIIENRLLQIRNFKDVNTPKLINVWSIRIDPRDSKKVIELIRNDFQKNDPVSLRHLKRIRKDIETSTLEVVLCSKEYICDEGEINNKLKSIWVGTKKYELSDDIEVPEFAPSTKELNNAWSVKYWPLIWNGNPNDQILNDYKIDMQEVRNELSRASTLSVKMATAGKQFPMVSVFVDPSRKKDKVVAEDGRNCENSLPIDHSVMVGIRAVGERLREGVDEDANSYLCLDYDVYLTHEPCSMCSMALIHSRVRRVVFLTEMQRTGSLKLTSGDGYCMNDNKQLNSTYEAFQWIGEEYPVGQVDRDVCC

SEQ ID NO: 202 - protein, 351 aa, Homo sapiens
MEPAPGLVEQPKCLEAGSPEPEPAPWQALPVLSEKQSGDVELVLAYAAPVLDKRQTSRLLKEVSALHPLPAQPHLKRVRPSRDAGSPHALEMLLCLAGPASGPRSLAELLPRPAVDPRGLGQPFLVPVPARPPLTRGQFEEARAHWPTSFHEDKQVTSALAGRLFSTQERAAMQSHMERAVWAARRAAARGLRAVGAVVVDPASDRVLATGHDCSCADNPLLHAVMVCVDLVARGQGRGTYDFRPFPACSFAPAAAPQAVRAGAVRKLDADEDGLPYLCTGYDLYVTREPCAMCAMALVHARILRVFYGAPSPDGALGTRFRIHARPDLNHRFQVFRGVLEEQCRWLDPDT

SEQ ID NO: 203 - protein, 349 aa, Rattus norvegicus
MEPTSGFAEQPGPEKVESEEQEPAQWQALPVLSEQQSGAVELVLAYAAPVLDKRQTSRLLREVSAVYPLPAQPHLKRVRPSRSAGGAHSSDLLLCLAGPSAGPRSLAELLPRPAVDPRGLGTPFLVPVPARPPLTRSQFEEARAHWPTSFHEDKQVTSALAGQLFSAQARAAMQTHMERAVRAAQRAAAQGLRAVGAVVVDPASDHVLATGHDCCSEASPLLHAVMVCIDLVAQGQGRGSCDLRRHPACSFTQATATQSARAGSVRKLDEDSLPYVCTGYDLYVTREPCVMCAMALVHARIQRVFYGAPSPDGALGTRFRVHARPDLNHRFQVFRGILEDQCRQLDPDP

SEQ ID NO: 204 - protein, 711 aa, Rattus norvegicus
MDIEDEENMSSSSIDVKENRNLDNMPPKDSSTPGPGEGIPLSNGGGGSTSRKRPLEEGSNGHSKYRLKKRRKTPGPVLPKNALMQLNEIKPGLQYMLLSQTGPVHAPLFVMSVEVNGQVFEGSGPTKKKAKLHAAEKALRSFVQFPNASEAHLAMGRTLSVNTDFTSDQADFPDTLFNGFETPDKSEPPFYVGSNGDDSFSSSGDVSLSASPVPASLTQPPLPIPPPFPPPSGKNPVMILNELRPGLKYDFLSESGESHAKSFVMSVVVDGQFFEGSGRNKKLAKARAAQSALATVFNLHLDQTPSRQPVLSEGLQLHLPQVLADAVSRLVLGKFSDLTDNFSSPHARRKVLSGVVMTTGTDVKDAKVISVSTGTKCINGEYMSDRGLALNDCHAEIISRRSLLRFLYAQLELYLNNKEDQKKSIFQKSERGGFRLKDTVQFHLYISTSPCGDARIFSPHEPVLEGMAPDSHQLTEPADRHPNRKARGQLRTKIESGEGTIPVRSNASIQTWDGVLQGERLLTMSCSDKIARWNVVGIQGALLSIFVEPIYFSSIILGSLYHGDHLSRAMYQRISNIEDLPPLYTLNKPLLSGISNAEARQPGKAPNFSVNWTVGDTAIEVINATTGKDELGRPSRLCKHALYCRWMRVHGKVPPHLLRTKITKPTTYHESKLAAKEYQAAKARLFTAFIKAGLGAWVEKPTEQDQFSFTP

SEQ ID NO: 205 - protein, 1226 aa, Homo sapiens
MNPRQGYSLSGYYTHPFQGYEHRQLRYQQPGPGSSPSSFLLKQIEFLKGQLPEAPVIGKQTPSLPPSLPGLRPRFPVLLASSTRGRQVDIRGVPRGVHLRSQGLQRGFQHPSPRGRSLPQRGVDCLSSHFQELSIYQDQEQRILKFLEELGEGKATTAHDLSGKLGTPKKEINRVLYSLAKKGKLQKEAGTPPLWKIAVSTQAWNQHSGVVRPDGHSQGAPNSDPSLEPEDRNSTSVSEDLLEPFIAVSAQAWNQHSGVVRPDSHSQGSPNSDPGLEPEDSNSTSALEDPLEFLDMAEIKEKICDYLFNVSDSSALNLAKNIGLTKARDINAVLIDMERQGDVYRQGTTPPIWHLTDKKRERMQIKRNTNSVPETAPAAIPETKRNAEFLTCNIPTSNASNNMVTTEKVENGQEPVIKLENRQEARPEPARLKPPVHYNGPSKAGYVDFENGQWATDDIPDDLNSIRAAPGEFRAIMEMPSFYSHGLPRCSPYKKLTECQLKNPISGLLEYAQFASQTCEFNMIEQSGPPHEPRFKFQVVINGREFPPAEAGSKKVAKQDAAMKAMTILLEEAKAKDSGKSEESSHYSTEKESEKTAESQTPTPSATSFFSGKSPVTTLLECMHKLGNSCEFRLLSKEGPAHEPKFQYCVAVGAQTFPSVSAPSKKVAKQMAAEEAMKALHGEATNSMASDNQPEGMISESLDNLESMMPNKVRKIGELVRYLNTNPVGGLLEYARSHGFAAEFKLVDQSGPPHEPKFVYQAKVGGRWFPAVCAHSKKQGKQEAADAALRVLIGENEKAERMGFTEVTPVTGASLRRTMLLLSRSPEAQPKTLPLTGSTFHDQIAMLSHRCFNTLTNSFQPSLLGRKILAAIIMKKDSEDMGVVVSLGTGNRCVKGDSLSLKGETVNDCHAEIISRRGFIRFLYSELMKYNSQTAKDSIFEPAKGGEKLQIKKTVSFHLYISTAPCGDGALFDKSCSDRAMESTESRHYPVFENPKQGKLRTKVENGEGTIPVESSDIVPTWDGIRLGERLRTMSCSDKILRWNVLGLQGALLTHFLQPIYLKSVTLGYLFSQGHLTRAICCRVTRDGSAFEDGLRHPFIVNHPKVGRVSIYDSKRQSGKTKETSVNWCLADGYDLEILDGTRGTVDGPRNELSRVSKKNIFLLFKKLCSFRYRRDLLRLSYGEAKKAARDYETAKNYFKKGLKDMGYGNWISKPQEEKNFYLCPV

SEQ ID NO: 206 - protein, 1175 aa, Rattus norvegicus
MSQGFRGPTGVFPHQTQPCLDPSYEHSKWRYLQPRGSESYLRSFQLQQIEFLKGRLPEAPLIGAQTQSLPPFLPGHWPRFPGPPAQGKQPEIWGFPRSVTLRNQGFHIGPPLPPPHSRGPPWRGAEGLCSHFQELSISQNPEQKVLNRLEELGEGKATTAYALARELRTPKKDINRILYSLERKGKLHRGVGKPPLWSLVPLSQACTQPPRAVNSDKEVPRGEPDLDSEDGDPASDLEGPSELLDMAEIKEKICDYLFNVSKSSALNLAKNIGLAKARDVNAVLIDLERQGDVYREGATPPIWYLTDKKRERLQMKRSTHSGPAATPAAVSEATQSTSFPTCHPPQSGGSSSMATSKRVENGQEPVTKYESRHEARPGPVRLRPHAYHNGPSRAGYVASENGPWATDDIPDNLNSIHTAPGEFRAIMEMPSFYSPTLPRCSPYKKLTECQLKNPVSGLLEYAQFTSQTCDFNLIEQSGPSHEPRFKFQVVINGREFPPAEAGSKKVAKQDAAVKAMAILLREAKAKDSGQPEELSNCPMEEDPEKPAESQPPSSSATSLFSGKSPVTTLLECMHKLGNSCEFRLLSKEGPAHDPKFQYCVAVGAQTFPSVSAPSKKVAKQMAAEEAMKALQEEAANSADDQSGGANTDSLDESVAPNKIRRIGELVRYLNTNPVGGLLEYARSHGFAAEFKLIDQSGPPHEPKFVYQAKVGGRWFPAVCAHSKKQGKQDAADAALRVLIGESEKAEQLGFAEVTPVTGASLRRTMLLLSRSPDAHPKTLPLTGSTFHDQIAMLSHRCFNALTNSFQPSLLGRKILAAIIMKRDPEDMGVVVSLGTGNRCVKGDSLSLKGETVNDCHAEIISRRGFIRFLYSELMKYNHHTAKNSIFELARGGEKLQIKKTVSFHLYISTAPCGDGAHFDKSCSDRAVESTESRHYPVFENPKQGKLRTKVENGEGTIPVESSDIVPTWDGIRLGERLRTMSCSDKILRWNVLGLQGALLTHFLQPVYLKSVTLGYLFSQGHLTRAICCRVTRDGNAFEDGLRYPFIVNHPKVGRVSVYDSKRQSGKTKETSVNWCLADGYDLEILDGTRGTVDGPGKELSRVSKKNIFLQFKKLCSFRARRDLLQLSYGEAKKAARDYDLAKNYFKKSLRDMGYGNWISKPQEEKNFYLCPVPND

SEQ ID NO: 207 - protein, 741 aa, Homo sapiens
MDIEDEENMSSSSTDVKENRNLDNVSPKDGSTPGPGEGSQLSNGGGGGPGRKRPLEEGSNGHSKYRLKKRRKTPGPVLPKNALMQLNEIKPGLQYTLLSQTGPVHAPLFVMSVEVNGQVFEGSGPTKKKAKLHAAEKALRSFVQFPNASEAHLAMGRTLSVNTDFTSDQADFPDTLFNGFETPDKAEPPFYVGSNGDDSFSSSGDLSLSASPVPASLAQPPLPVLPPFPPPSGKNPVMILNELRPGLKYDFLSESGESHAKSFVMSVVVDGQFFEGSGRNKKLAKARAAQSALAAIFNLHLDQTPSRQPIPSEGLQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEGSRSYTQAGVQWCNHGSLQPRPPGLLSDPSTSTFQGAGTTEPADRHPNRKARGQLRTKIESGEGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTP

SEQ ID NO: 208 - protein, 711 aa, Mus musculus
MDIEDEENMSSSSTDIKENRNLDNMPPKDSSTPGPGEGIPLSNGGGGSTSRKRPLEEGSNGHSKYRLKKRRKTPGPVLPKNALMQLNEIKPGLQYMLLSQTGPVHAPLFVMSVEVNGQVFEGSGPTKKKAKLHAAEKALRSFVQFPNASEAHLAMGRTLSVNTDFTSDQADFPDTLFNGFETPDKSEPPFYVGSNGDDSFSSSGDVSLSASPVPASLTQPPLPIPPPFPPPSGKNPVMILNELRPGLKYDFLSESGESHAKSFVMSVVVDGQFFEGSGRNKKLAKARAAQSALATVFNLHLDQTPSRQPVLSEGLQLHLPQVLADAVSRLVLGKFSDLTDNFSSPHARRKVLSGVVMTTGTDVKDAKVISVSTGTKCINGEYMSDRGLALNDCHAEIISRRSLLRFLYAQLELYLNNKEDQKKSIFQKSERGGFRLKDTVQFHLYISTSPCGDARIFSPHEPVLEGMTPDSHQLTEPADRHPNRKARGQLRTKIESGEGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDATIEVINATTGKDELGRPSRLCKHALYCRWMRVHGKVPPHLLRTKITKPTTYHESKLAAREYQAAKARLFTAFIKAGLGAWVEKPTEQDQFSFTP

SEQ ID NO: 209 - protein, 1178 aa, Mus musculus
MSQGFRGPTGVFPHQTQSYLDPSHEHSKWRYPQPQGPESYPRSFQLQQIEFLKGRLPEAPLIGIQTQSLPPFLPGHWPRFPGPPAQDRQLEIWEFPRSVTLRNQGFHIGPPLPPPHSRGTPWRGADGLCSHFRELSISQSPEQKVLNRLEELGEGKATTAHVLARELRIPKRDINRILYSLEKKGKLHRGRGKPPLWSLVPLSQAWTQPPGVVNPDSCIQEFPRGEPGLDSEDGDPASDLEGPSEPLDMAEIKEKICDYLFNVSNSSALNLAKNIGLTKARDVTSVLIDLERQGDVYRQGATPPIWYLTDKKRERLQMKRSTHSAPAPTPTAVPEATRSPSFPACHPPPAGASSSVAASKRVENGQEPAIKHESRHEARPGPMRLRPHAYHNGPSRAGYVASENGQWATDDIPDNLNSIHTAPGEFRAIMEMPSFYSPTLPRCSPYKKLTECQLKNPVSGLLEYAQFTSQTCDFNLIEQSGPSHEPRFKFQVVINGREFPPAEAGSKKVAKQDAAVKAMAILLREAKAKDSGQPEDLSHCPMEEDSEKPAEAQAPSSSATSLFSGKSPVTTLLECMHKLGNSCEFRLLSKEGPAHDPKFQYCVAVGAQTFPPVSAPSKKVAKQMAAEEAMKALQEEAASSADDQSGGANTDSLDESMAPNKIRRIGELVRYLNTNPVGGLLEYARSHGFAAEFKLIDQSGPPHEPKFVYQAKVGGRWFPAVCAHSKKQGKQDAADAALRVLIGESEKAEQLGFAEVTPVTGASLRRTMLLLSRSPDAHPKTLPLSGSTFHDQIAMLSHRCFNALTNSFQPSLLGRKILAAIIMKRDPEDMGVVVSLGTGNRCVKGDSLSLKGETVNDCHAEIISRRGFIRFLYSELMKYNHHTAKNSIFELARGGEKLQIKKTVSFHLYISTAPCGDGALFDKSCSDRAVESTESRHYPVFENPKQGKLRTKVENGEGTIPVESSDIVPTWDGIRLGERLRTMSCSDKILRWNVLGLQGALLTHFLQPVYLKSVTLGYLFSQGHLTRAICCRVTRDGKAFEDGLRYPFIVNHPKVGRVSVYDSKRQSGKTKETSVNWCMADGYDLEILDGTRGTVDGPGKELSRVSKKNIFLQFKKLCSFRARRDLLQLSYGEAKKAARDYDLAKNYFKKSLRDMGYGNWISKPQEEKNFYLCPVPND

SEQ ID NO: 210 - protein, 147 aa, Homo sapiens
MKQLFPPPPGTSLTHALGAWRGRERAQAATSLLASSASQFPTAVEDALMSVLTSHCAPSTPAATRAQQTGTRGHIHPACPCQQSCVGASRPPGRPQIFLPLTTALSLEAYAADTCSAADFLHNPSSWGKVWYLNEASFDLYSYHYFW

SEQ ID NO: 211 - protein, 746 aa, Rattus norvegicus
MASVLGSGRGSGGLSSQLKCKSKRRRRRRSKRKDKVSILSTFLAPFKYLSPGTTNTEDEDNLSTSSAEVKENRNVSNLGTRPLPPGDWARGGSTPSVKRKRPLEEGNGGHFCKLQLIWKKLSWSMTPKNALVQLHELKPGLQYRMVSQTGPVHAPVFAVAVEVNGLTFEGTGPTKKKAKMRAAEMALKSFVQFPNAFQAHLAMGSSTSPCTDFTSDQADFPDTLFKEFEPSSRNEDFPGCCPVDTEFLSSAYRRGRLLYHTLDLMGQALPDRSRLAPGALGERNPVVVLNELRSGLRYVCLSETAEKPRVKSFVMAVCVDGRTFEGSGRSKKLAKGQAAQAALQALFDIRLPGHIPSRSKSNLLPQDFADSVSQLVTQKFRELTVGLTSVYARHKTLAGIVMTKGLDTKQAQVIVLSSGTKCISGEHISDQGLVVNDCHAEIVARRAFLHFLYTQLELHLSKHQEDPERSIFIRVKEGGYRLRENILFHLYVSTSPCGDARLNSPYEITIDLNSSKHIVRKFRGHLRTKIESGEGTVPVRGPSAVQTWDGILLGEQLVTMSCTDKIASWNVLGLQGALLCHFIEPVYLHSIIVGSLHHTGHLARVMSHRMEGIGQLPASYRQNRPLLSGVSNAEARQPGKSPHFSANWVVGSADLEIINATTGKRSCGGSSRLCKHVFSARWARLHGRLSTRIPGHGDTPSMYCEAKRGAHTYQSVKQQLFKAFQKAGLGTWVRKPPEQDQFLLSL

SEQ ID NO: 212 - protein, 745 aa, Mus musculus
MASVLGSGRGSGGLSSQLKCKSKRRRRRRSKRKDKVSILSTFLAPFKYLSPGTTNTEDEDNLSTSSAEVKENRNVSNLGTRPLPPGDWARGSTPSVKRKRPLEEGNGGHFCKLQLIWKKLSWSVTPKNALVQLHELKPGLQYRMVSQTGPVHAPVFAVAVEVNGLTFEGTGPTKKKAKMRAAEMALKSFVQFPNAFQAHLAMGSSTSPCTDFTSDQADFPDTLFKEFEPSSKNEDFPGCHPVDTEFLSSAYRRGRLLYHTLDLMGQALPDRSRLAPGALGERNPVVVLNELRSGLRYVCLSETAEKPRVKSFVMAVCVDGRTFEGSGRSKKLAKGQAAQAALQALFDIRLPGHIPSRSKSNLLPQDFADSVSQLVTQKFRELTVGLTSVYARHKTLAGIVMTKGLDTKQAQVIVLSSGTKCISGEHISDQGLVVNDCHAEIVARRAFLHFLYSQLELHLSKHQEDPERSIFIRLKEGGYRLRENILFHLYVSTSPCGDARVNSPYEITTDLNSSKHIVRKFRGHLRTKIESGEGTVPVRGPSAVQTWDGILLGEQLITMSCTDKIASWNVLGLQGALLCHFIEPVYLHSIIVGSLHHTGHLARVMSHRMEGIGQLPASYRQNRPLLSGVSHAEARQPGKSPHFSANWVVGSADLEIINATTGKRSCGGSSRLCKHVFSAWWARLHGRLSTRIPSHGDTPSMYCEAKQGAHTYQSVKQQLFKAFQKAGLGTWVRKPPEQDQFLLSL

SEQ ID NO: 213 - protein, 739 aa, Homo sapiens
MASVLGSGRGSGGLSSQLKCKSKRRRRRRSKRKDKVSILSTFLAPFKHLSPGITNTEDDDTLSTSSAEVKENRNVGNLAARPPPSGDRARGGAPGAKRKRPLEEGNGGHLCKLQLVWKKLSWSVAPKNALVQLHELRPGLQYRTVSQTGPVHAPVFAVAVEVNGLTFEGTGPTKKKAKMRAAELALRSFVQFPNACQAHLAMGGGPGPGTDFTSDQADFPDTLFQEFEPPAPRPGLAGGRPGDAALLSAAYGRRRLLCRALDLVGPTPATPAAPGERNPVVLLNRLRAGLRYVCLAEPAERRARSFVMAVSVDGRTFEGSGRSKKLARGQAAQAALQELFDIQMPGHAPGRARRTPMPQEFADSISQLVTQKFREVTTDLTPMHARHKALAGIVMTKGLDARQAQVVALSSGTKCISGEHLSDQGLVVNDCHAEVVARRAFLHFLYTQLELHLSKRREDSERSIFVRLKEGGYRLRENILFHLYVSTSPCGDARLHSPYEITTDLHSSKHLVRKFRGHLRTKIESGEGTVPVRGPSAVQTWDGVLLGEQLITMSCTDKIARWNVLGLQGALLSHFVEPVYLQSIVVGSLHHTGHLARVMSHRMEGVGQLPASYRHNRPLLSGVSDAEARQPGKSPPFSMNWVVGSADLEIINATTGRRSCGGPSRLCKHVLSARWARLYGRLSTRTPSPGDTPSMYCEAKLGAHTYQSVKQQLFKAFQKAGLGTWVRKPPEQQQFLLTL

SEQ ID NO: 214 - protein, 747 aa, Rattus norvegicus
MPLFKLTGQGKQIDDAMRSFAEKVFASEVKDEGGRHEISPFDVDEICPISLREMQAHIFHMENLSMSMDGRRKRRFQGRKTVNLSIPQSETSSTKLSHIEEFISSSPTYESVPDFQRVQITGDYASGVTVEDFEVVCKGLYRALCIREKYMQKSFQRFPKTPSKYLRNIDGEALVAIESFYPVFTPPPKKGEDPFRREDLPANLGYHLKMKGGVIYIYPDEAAASRDEPKPYPYPNLDDFLDDMNFLLALIAQGPVKTYTHRRLKFLSSKFQVHQMLNEMDELKELKNNPHRDFYNCRKVDTHIHAAACMNQKHLLRFIKKSYHIDADRVVYSTKEKNLTLKELFAQLNMHPYDLTVDSLDVHAGRQTFQRFDKFNDKYNPVGASELRDLYLKTDNYINGEYFATIIKEVGADLVDAKYQHAEPRLSIYGRSPDEWSKLSSWFVGNRIYCPNMTWMIQVPRIYDVFRSKNFLPHFGKMLENIFLPVFEATINPQTHPDLSVFLKHITGFDSVDDESKHSGHMFSSKSPKPEEWTMENNPSYTYYAYYMYANIMVLNCLRKERGMNTFLFRPHCGEAGALTHLMTAFMIADNISHGLNLKKSPVLQYLFFLAQIPIAMSPLSNNSLFLEYAKNPFLDFLQKGLMISLSTDDPMQFHFTKEPLMEEYAIAAQVFKLSTCDMCEVARNSVLQCGISHEEKAKFLGNNYLEEGPVGNDIRRTNVAQIRMAYRYETWCYELNLIAEGLKSTE

SEQ ID NO: 215 - protein, 747 aa, Homo sapiens
MPLFKLPAEEKQIDDAMRNFAEKVFASEVKDEGGRQEISPFDVDEICPISHHEMQAHIFHLETLSTSTEARRKKRFQGRKTVNLSIPLSETSSTKLSHIDEYISSSPTYQTVPDFQRVQITGDYASGVTVEDFEIVCKGLYRALCIREKYMQKSFQRFPKTPSKYLRNIDGEAWVANESFYPVFTPPVKKGEDPFRTDNLPENLGYHLKMKDGVVYVYPNEAAVSKDEPKPLPYPNLDTFLDDMNFLLALIAQGPVKTYTHRRLKFLSSKFQVHQMLNEMDELKELKNNPHRDFYNCRKVDTHIHAAACMNQKHLLRFIKKSYQIDADRVVYSTKEKNLTLKELFAKLKMHPYDLTVDSLDVHAGRQTFQRFDKFNDKYNPVGASELRDLYLKTDNYINGEYFATIIKEVGADLVEAKYQHAEPRLSIYGRSPDEWSKLSSWFVCNRIHCPNMTWMIQVPRIYDVFRSKNFLPHFGKMLENIFMPVFEATINPQADPELSVFLKHITGFDSVDDESKHSGHMFSSKSPKPQEWTLEKNPSYTYYAYYMYANIMVLNSLRKERGMNTFLFRPHCGEAGALTHLMTAFMIADDISHGLNLKKSPVLQYLFFLAQIPIAMSPLSNNSLFLEYAKNPFLDFLQKGLMISLSTDDPMQFHFTKEPLMEEYAIAAQVFKLSTCDMCEVARNSVLQCGISHEEKVKFLGDNYLEEGPAGNDIRRTNVAQIRMAYRYETWCYELNLIAEGLKSTE

SEQ ID NO: 216 - protein, 26 aa, Oryctolagus cuniculus
MNQKHLLRFIKKSYQVDADRVVYSTK

SEQ ID NO: 217 - protein, 26 aa, Gallus gallus
MNQKHLLRFIKKSYRVDADRVVYDAK

SEQ ID NO: 218 - protein, 745 aa, Mus musculus
MPLFKLTGQGKQIDDAMRSFAEKVFASEVKDEGGRHEISPFDVDEICPISLHEMQAHIFHMENLSMDGRRKRRFQGRKTVNLSIPQSETSSTKLSHIEEFISSSPTYESVPDFQRVQITGDYASGVTVEDFEVVCKGLYRALCIREKYMQKSFQRFPKTPSKYLRNIDGEALVGNESFYPVFTPPPKKGEDPFRTEDLPANLGYHLKMKAGVIYIYPDEAAANRDEPKPYPYPNLDDFLDDMNFLLALIAQGPVKTYAHRRLKFLSSKFQVHQMLNEMDELKELKNNPHRDFYNCRKVDTHIHAAACMNQKHLLRFIKKSYHIDADRVVYSTKEKSLTLKELFAKLNMHPYDLTVDSLDVHAGRQTFQRFDKFNDKYNPVGASELRDLYLKTDNYINGEYFATIIKEVGADLVEAKYQHAEPRLSIYGRSPDEWNKLSSWFVCNRIYCPNMTWMIQVPRIYDVFRSKNFLPHFGKMLENIFLPVFEATINPQAHPDLSVFLKHITGFDSVDDESKHSGHMFSSKSPKPEEWTMENNPSYTYYAYYMYANITVLNSLRKERGMNTFLFRPHCGEAGALTHLMTAFMIADNISHGLNLKKSPVLQYLFFLAQIPIAMSPLSNNSLFLEYAKNPFLDFLQKGLMISLSTDDPMQFHFTKEPLMEEYAIAAQVFKLSTCDMCEVARNSVLQCGISHEEKAKFLGNNYLEEGPVGNDIRRTNVAQIRMAYRYETWCYELNLIAEGLKATE

SEQ ID NO: 219 - protein, 790 aa, Dictyostelium discoideum Skipper virus
MSTPLRGSSPQVSFYESELDQEGGSDASHFTYRNYMEDDKINSFTFNMARKDQTQLFQRIILTNESESEIEEYAEVAEQLLDAINLREKYVFHPKIWKADAPVGEKPPYSPFESDESTNCATEHMFKEVNGVYFVYSNETDMKSNKALFSVPHTLASYYKDINNLMMLSSYGPAKTFTFKRLQLLESKFNMHTLLNDSLELFQQKTAPHRDFYNVRKVDTHVHHSSSMNQKHLLKFIKRKLKENPNEIVIFRDDKYLTLAEVFKSLNLDVDELSVDTLDVHADNNTFHRFDKFNLKYNPCGQSRLREIFLKTDNLIKGKYLAEISKEVFTDLESSKYQCAEYRLSIYGRKMSEWDTLASWIVDNDLFSTKVRWLIQVPRLYDVYRETSTTTFQDFLNNVFHPLFEVTKDPSSHPKLHLFLQQVVGIDCVDDESKFEKKFTEKFPVPGEWSSEHNPPYTYYLYYLYANLYTLNQFREEKGLNILTLRPHSGEAGEVDHMGAAFYLAHGINHGINLRKTPVLQYLYYLTQIGIAMSPLSNNSLFLTYNRNPFPAFFARGLNVSISTDDPLQFHYTKEPLMEEYSIATQVWRLSVCDICEIARNSVLQSGFEHNVKSHWLGPDYANSGGNDIKKTNISDIRVCFRNETLIEELHLILKSLQTLPNFKNLNINFLLDKLPSEITTGNDYKLKKAQLKLNGANKLRNSSVGSTPNNGTPSSSGTPSLSSPGAIVHLMKTKPYIPPPLSLNIKQENNNNNNNNNNNNNNNNNNNTNTNTNSNSTTTNQDDNSKSDK

SEQ ID NO: 220 - protein, 767 aa, Homo sapiens
MPRQFPKLNISEVDEQVRLLAEKVFAKVLREEDSKDALSLFTVPEDCPIGQKEAKERELQKELAEQKSVETAKRKKSFKMIRSQSLSLQMPPQQDWKGPPAASPAMSPTTPVVTGATSLPTPAPYAMPEFQRVTISGDYCAGITLEDYEQAAKSLAKALMIREKYARLAYHRFPRITSQYLGHPRADTAPPEEGLPDFHPPPLPQEDPYCLDDAPPNLDYLVHMQGGILFVYDNKKMLEHQEPHSLPYPDLETYTVDMSHILALITDGPTKTYCHRRLNFLESKFSLHEMLNEMSEFKELKSNPHRDFYNVRKVDTHIHAAACMNQKHLLRFIKHTYQTEPDRTVAEKRGRKITLRQVFDGLHMDPYDLTVDSLDVHAGRQTFHRFDKFNSKYNPVGASELRDLYLKTENYLGGEYFARMVKEVARELEESKYQYSEPRLSIYGRSPEEWPNLAYWFIQHKVYSPNMRWIIQVPRIYDIFRSKKLLPNFGKMLENIFLPLFKATINPQDHRELHLFLKYVTGFDSVDDESKHSDHMFSDKSPNPDVWTSEQNPPYSYYLYYMYANIMVLNNLRRERGLSTFLFRPHCGEAGSITHLVSAFLTADNISHGLLLKKSPVLQYLYYLAQIPIAMSPLSNNSLFLEYSKNPLREFLHKGLHVSLSTDDPMQFHYTKEALMEEYAIAAQVWKLSTCDLCEIARNSVLQSGLSHQEKQKFLGQNYYKEGPEGNDIRKTNVAQIRMAFRYETLCNELSFLSDAMKSEEITALTN

SEQ ID NO: 221 - protein, 879 aa, Homo sapiens
MRNRGQGLFRLRSRCFLHQSLPLGAGRRKGLDVAEPGPSRCRSDSPAVAAVVPAMASYPSGSGKPKAKYPFKKRASLQASTAAPEARGGLGAPPLQSARSLPGPAPCLKHFPLDLRTSMDGKCKEIAEELFTRSLAESELRSAPYEFPEESPIEQLEERRQRLERQISQDVKLEPDILLRAKQDFLKTDSDSDLQLYKEQGEGQGDRSLRERDVLEREFQRVTISGEEKCGVPFTDLLDAAKSVVRALFIREKYMALSLQSFCPTTRRYLQQLAEKPLETRTYEQGPDTPVSADAPVHPPALEQHPYEHCEPSTMPGDLGLGLRMVRGVVHVYTRREPDEHCSEVELPYPDLQEFVADVNVLMALIINGPIKSFCYRRLQYLSSKFQMHVLLNEMKELAAQKKVPHRDFYNIRKVDTHIHASSCMNQKHLLRFIKRAMKRHLEEIVHVEQGREQTLREVFESMNLTAYDLSVDTLDVHADRNTFHRFDKFNAKYNPIGESVLREIFIKTDNRVSGKYFAHIIKEVMSDLEESKYQNAELRLSIYGRSRDEWDKLARWAVMHRVHSPNVRWLVQVPRLFDVYRTKGQLANFQEMLENIFLPLFEATVHPASHPELHLFLEHVDGFDSVDDESKPENHVFNLESPLPEAWVEEDNPPYAYYLYYTFANMAMLNHLRRQRGFHTFVLRPHCGEAGPIHHLVSAFMLAENISHGLLLRKAPVLQYLYYLAQIGIAMSPLSNNSLFLSYHRNPLPEYLSRGLMVSLSTDDPLQFHFTKEPLMEEYSIATQVWKLSSCDMCELARNSVLMSGFSHKVKSHWLGPNYTKEGPEGNDIRRTNVPDIRVGYRYETLCQELALITQAVQSEMLETIPEEAGITMSPGPQ

SEQ ID NO: 222 - protein, 798 aa, Mus musculus
MASEARSGLGASPLQSARSLPGNAPCLKHFPLDLRTSMDGKCKEIAEELFSRSLAESELRSAPYEFPEESPIEQLEERRQRLERQISQDVKLEPDILLRAKQDFLKTDSDSDLQLYKEQGEGQGDRGLWERDVVLEREFQRVIISGEEKCGVPFTDLLDAAKSVVRALFIREKYMALSLQSFCPTTRRYLQQLAEKPLETRTYEQSPDTPVSADAPVHPPALEQHPYEHCEPSAMPGDLGLGLRMVRGVVHVYTRRDPDEHCPEVELPYPDLQEFVADVNVLMALIINGPIKSFCYRRLQYLSSKFQMHVLLNEMKELAAQKKVPHRDFYNIRKVDTHIHASSCMNQKHLLRFIKRAMKRHLEEIVHVEQGREQTLREVFESMNLTAYDLSVDTLDVHADRNTFHRFDKFNAKYNPIGESVLREIFIKTDNKISGKYFAHIIKEVMADLEESKYQNAELRLSIYGRSRDEWDKLARWAVNHKVHSPNVRWLVQVPRLFDVYRTKGQLANFQEMLENIFLPLFEATVHPASHPELHLFLEHVDGFDSVDDESKPENHVFNLESPLPEAWVEEDNPPYAYYLYYTFANMAMLNHLRRQRGFHTFVLRPHCGEAGPIHHLVSAFMLAENISHGLLLRKAPVLQYLYYLAQIGIAMSPLSNNSLFLSYHRNPLPEYLSRGLMVSLSTDDPLQFHFTKEPLMEEYSIATQVWKLSSCDMCELARNSVLMSGFSHKVKSHWLGPNYTKEGPEGNDIRRTNVPDIRVGYRYETLCQELALITQAVQSEMLETIPEEVGIVMSPGP

SEQ ID NO: 223 - protein, 824 aa, Rattus norvegicus
MASYPGPGKSKAKYPFKKRASLQASAAAPEARSGLGASPLQSARSLPGTAPCLKHFPLDLRTSMDGKCKEIAEELFSRSLAESELRSAPYEFPEESPIEQLEERRQRLERQISQDVKLEPDILLRAKQDFLKTDSDSDLQLYKEQGEGQGDRGLWERDVVLEREFQRVIISGEEKCGVPFTDLLDAAKSVVRALFIREKYMALSLQSFCPTTRRYLQQLAEKPLETRTYEQSPDTPVSADAPVHPPALEQHPYEHCEPSTMPGDLGLGLRMVRGVVHVYTRRDPDEHCPEVELPYPDLQEFVADVNVLMALIINGPIKSFCYRRLQYLSSKFQMHVLLNEMKELAAQKKVPHRDFYNIRKVDTHIHASSCMNQKHLLRFIKRAMKRHLEEIVHVEQGREQTLREVFESMNLTAYDLSVDTLDVHADRNTFHRFDKFNAKYNPIGESVLREIFIKTDNKISGKYFAHIIKEVMSDLEESKYQNAELRLSIYGRSRDEWDKLARWAVNHRVHSPNVRWLVQVPRLFDVYRTKGQLANFQEMLENIFLPLFEATVHPASHPELHLFLEHVDGFDSVDDESKPENHVFNLESPLPEAWVEEDNPPYAYYLYYTFANMAMLNHLRRQRGFHTFVLRPHCGEAGPIHHLVSAFMLAENISHGLLLRKAPVLQYLYYLAQIGIAMSPLSNNSLFLSYHRNPLPEYLSRGLMVSLSTDDPLQFHFTKEPLMEEYSIATQVWKLSSCDMCELARNSVLMSGFSHKVKSHWLGPNYTKEGPEGNDIRRTNVPDIRVGYRYETLCQELALITQAVQSEMLETIPEEVGIVMSPGP

SEQ ID NO: 224 - protein, 362 aa, Plasmodiophora sp.
  source 1..362: note = Plasmodium gallinaceum
MTILHEEINFLKKDELNINLKCLDKKERYKIWRRIPKCELHCHLDLCFSLEFFLKCVRKYNLQPDLTDDEVVDYYLFKDKGKSLNEFIERSRRVTDIFINYDIIKDIAKNAVFNKYKEGVILIEFRYSPSYIAYKYNLCIDLIHKTIVEGINEAVEKLNHKIHVGLICIGETGISEESLRKAAEFCVKNKKDFVGFDHAGHERDLKPYKEIYDYVRENGIPLTIHAGEDLTLPNLNTIYSAIEVLKAKRIGHGIRVIESEDLINLIKKNDILLEICPISNLLLNNVKSMDTHPIKKLYDSGIKVSVNTDDPGMFLTEINDEYEELYLNLNFNLEDFMKMNLWALEKSFVKSEIKDKLKKLYF

SEQ ID NO: 225 - protein, 363 aa, Plasmodiophora sp.
  source 1..363: note = Plasmodium knowlesi
MNILQEPIDFLKKDELKNIDLSQMDKKERYKIWKRIPKCELHCHLDLCFSADFFLSCVRKYNLQPNLSDEEVLDYYLFAKGGKSLGEFVEKAIRVADIFQDYEMIEDLAKHAVFNKYKEGVVLMEFRYSPTFVAFKHNLDIELIHQAIVKGIKEVVELLDHKIDVTLLCIGDTGHRAADIKASADFCLKHKADFVGFDHGGHEVDLKPYKEIFDYVKEGGMHLTVHAGEDVTLPNLNTLYSAIQVLKVERIGHGIRVSESQELIDMVKENNILLEVCPISNVLLKNAKSFDTHPIRKLYDAGVKVSVSSDDPGMFLTNINDDYEKLYTHLHFTLEDFMKMNEWALEKSFIGCDIKEKIKKLYF

SEQ ID NO: 226 - protein, 363 aa, Plasmodiophora sp.
  source 1..363: note = Plasmodium berghei
MEIPNEEIKFLKKEDIKNINLNGMNKKERYEIWKKIPKVELHCHLDLTFSGKFFLKWVRKYNLQPNMTDDQVLDHYLFTKEGKSLAEFIRKAISVSDIYRDYDILEDLAKWAVIEKYKEGVVLMEFRYSPTFVSSSHGLDIELIHKAFVKGIKNATEMLNNKIYVALICISDTGHSAASIKHSGDFAIKHKHDFVGFDHGGREIDLKDHKDVYHSVRNHGLHLTVHAGEDATLPNLNTLYTAINILNVERIGHGIRVSESEELIELVKKNNILLEVCPISNLLLNNVKSMDTHPIRKLFDAGVKVSVNSDDPGMFLTDINDNYEKLYIHLNFTLEEFMTMNNWALEKSFVNDDIKSKLKTMYF

SEQ ID NO: 227 - protein, 363 aa, Plasmodiophora sp.
  source 1..363: note = Plasmodium vivax
MNILQEPIDFLKKEELKNIDLSQMSKKERYKIWKRIPKCELHCHLDLCFSADFFVSCIRKYNLQPNLSDEEVLDYYLFAKGGKSLGEFVEKAIKVADIFHDYEVIEDLAKHAVFNKYKEGVVLMEFRYSPTFVAFKYNLDIELIHQAIVKGIKEVVELLDHKIHVALMCIGDTGHEAANIKASADFCLKHKADFVGFDHGGHEVDLKEYKEIFDYVRESGVPLSVHAGEDVTLPNLNTLYSAIQVLKVERIGHGIRVAESQELIDMVKEKNILLEVCPISNVLLKNAKSMDTHPIRQLYDAGVKVSVNSDDPGMFLTNINDDYEELYTHLNFTLEDFMKMNEWALEKSFMDSNIKDKIKNLYF

SEQ ID NO: 228 - protein, 363 aa, Plasmodiophora sp.
  source 1..363: note = Plasmodium cynomolgi
MNILQEPIDFLKKDEIKNIDLSQMSKKERYKIWKRIPKCELHCHLDLCFSADFFLSCIRKYNLQPNLSDEEVLDYYLFAKGGKSLGEFVEKAIRVADIFHDYEVIEDLAKHAVFNKYKEGVVLMEFRYSPTFVAFKYKLDIELIHQAIVKGIKEVVELLDHKIHVALMCIGDTGHEAANIKASADFCLKHRADFVGFDHGGHEVDLKQYKEIFDYVRESGIPLSVHAGEDVTLPNLNTLYSAIQVLKVERIGHGIRVSESQELIDMVKEKNILLEVCPISNVLLKNAKSMDTHPIRQLYDAGVKVSVNSDDPGMFLTNINDDYEELYTHLNFTLEDFMKMNEWALEKSFMDSNIKDKVKNLYF

SEQ ID NO: 229 - protein, 363 aa, Homo sapiens
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 230 - protein, 352 aa, Mus musculus
MAQTPAFNKPKVELHVHLDGAIKPETILYFGKKRGIALPADTVEELRNIIGMDKPLSLPGFLAKFDYYMPVIAGCREAIKRIAYEFVEMKAKEGVVYVEVRYSPHLLANSKVDPMPWNQTEGDVTPDDVVDLVNQGLQEGEQAFGIKVRSILCCMRHQPSWSLEVLELCKKYNQKTVVAMDLAGDETIEGSSLFPGHVEAYEGAVKNGIHRTVHAGEVGSPEVVREAVDILKTERVGHGYHTIEDEALYNRLLKENMHFEVCPWSSYLTGAWDPKTTHAVVRFKNDKANYSLNTDDPLIFKSTLDTDYQMTKKDMGFTEEEFKRLNINAAKSSFLPEEEKKELLERLYREYQ

SEQ ID NO: 231 - protein, 363 aa, Bos taurus
MAQTPAFNKPKVELHVHLDGAIKPETILYYGRKRGIALPADTPEELQNIIGMDKPLSLPEFLAKFDYYMPAIAGCREAVKRIAYEFVEMKAKDGVVYVEVRYSPHLLANSKVEPIPWNQAEGDLTPDEVVSLVNQGLQEGERDFGVKVRSILCCMRHQPSWSSEVVELCKKYREQTVVAIDLAGDETIEGSSLFPGHVKAYAEAVKSGVHRTVHAGEVGSANVVKEAVDTLKTERLGHGYHTLEDATLYNRLRQENMHFEVCPWSSYLTGAWKPDTEHPVVRFKNDQVNYSLNTDDPLIFKSTLDTDYQMTKNEMGFTEEEFKRLNINAAKSSFLPEDEKKELLDLLYKAYGMPSPASAEQCL

SEQ ID NO: 232 - protein, 357 aa, Gallus gallus
MERGVRVFGEPKVELHIHLDGAIRPETILHFGKKRGVPLPGSTVDELMKHVSYQTPLSLKLFLEKFNHYMPAIAGDREAVRRIAYELVETKAKEGVVYVEVRYSPHLLANCRVEPIPWGQAEGDLTPEEVVNLVNQGLQDGERNFRIKARSILCCMRHMPSWSPEVVELCKKYQNNSVVAIDLAGDELLMASSDHKAAYEEAERCGIHRTVHAGEAGPATMIKEAVYLLKAERIGHGYHVLEDPELYRELLRTRMHFEVCPWSSYLTGACLPDFRKHPVVQFKKDQANYSINTDDPLIFNSNIDKDYGIVKEYMDFTEEDFKRVNINAAQSSFLPEKEKQELLNTLYEAYGMVPATS

SEQ ID NO: 233 - protein, 359 aa, Danio rerio
MNGKPAFDKPKVELHVHLDGAIRLKTVLDVAKRRGISLPVSMEEELKELCTVNEPATLTEFLGKFSHFMHVIAGDREAIKRIAYEFVETKAKEGVIYVEARYSPHFLANKGVEPLPWDQKPGDITPDDVVDLVNQGFKEGEQAFKTKARSILCCMRHMPNWSMEVVELCKKFHKDGVVAIDLAGDESMNCESYPGHKKAFEEAVRSNVHRTVHAGEVGPASVVREAVEVLKAERIGHGYHTLEDQNLYKQLLHQNMHFEMCPVSSRLTGACEPDFTKHPLITFKKDKANYSLNTDDPTIFNSTLNSDYEVVQKYMDFTEEEFKRLNINAAKSCFLPEKEKEKLLNQLYEAYGMRKSTSF

SEQ ID NO: 234 - protein, 358 aa, Xenopus laevis
MESKAFNKPKVELHVHLDGSIKPETIIHFAKKRQIKLPADTVEGLLEHVSYKEPLSLTEFLQKFNHYMPAIAGDREAIKRIAYEFVEMKAKEGVIYVEVRYSPHFLANSKVDPIPWGQKEGDITPDEVVDLVNQGLRKGEKTFNIKARSILCCMRHMPNWSSEVIELCKKYQNDTVVAIDLAGDESLNCESYPGHRKAYEEAVKCGIHRTVHAGEVGPPSVVKEAVEVLKAERIGHGYHTTEDPNLYKELLENNMHFEVCPWSSYLTSACHPDFTKHPATQFRKDKANFSLNTDDPLIFGSTLDVDYSIAVQHMGFTEDEFKRVNINAAKSSFLPDNEKKELLYKLYEAYGMILSTGL

SEQ ID NO: 235 - protein, 367 aa, Plasmodiophora sp.
  source 1..367: note = Plasmodium falciparum
MNCKNMDTSYEIINYLTKDELDIDLSCMDKKERYKIWKRLPKCELHCHLDVCFSVDFFLNVIRKYNIQPNMSDEEIIDYYLFSKPGKSLDEFVEKALRLTDIYIDYTVVEDLAKHAVFNKYKEGVVLMEFRYSPSFMSFKHNLDKDLIHEAIVKGLNEAVALLEYKIQVGLLCTGDGGLSHERMKEAAEFCIKHKKDFVGYDHAGHEVDLKPFKDIFDNIREEGISLSVHAGEDVSIPNLNSLYTAINLLHVKRIGHGIRVSESQELIDLVKEKDILLEVCPISNVLLNNVKSMDTHPIRMLYDAGVKVSVNSDDPGMFLTNITDNYEELYTHLNFTLADFMKMNLWAVQKSFVDPDIKNKIISKYF

SEQ ID NO: 236 - protein, 337 aa, Drosophila melanogaster
MEQFLKGLPKVELHAHLNGSLGIKSLCDLGERLYGTSCKDFLKLCAHFSRFEKDMDACFEKFAFVHELTSTREGLRFATELAIRDFAEDNVQYVEMRTTPKANENYSRRDYLQIVIDAIKAASETYPEITVKLLPSINRAEPVDVAEETVSLAVELARAHPNLILGIDLSGNPGKGRFSDFAPILAQARDKGLKLAIHCAEIENPSEVKEMLHFGMSRCGHGTFLTPEDIGQLKQRNIAIECCLTSNVKSGTVPSLEEHHLKRIMEADAPKVICTDDSGVFDTTLTKEFLIAAETFGLTREQCIDLTLEAVHHSFASEQEQIQMADRVGNYADILVK

SEQ ID NO: 237 - protein, 358 aa, Xenopus tropicalis
MESKAFNKPKVELHVHLDGSIKPETIIHFAKKRQIKLPADTVEGLLEHVSYKEPLSLTEFLSKFNHYMPAIAGDREAIKRIAYEFVEMKAKEGVIYVEVRYSPHFLANSKVEPIPWGQKEGDITPDEVVDLVNQGLRKGEKAFNIKARSILCCMRHMPSWSTEVVELCKKYQNDTVVAIDLAGDESLNCESYPGHRKAYEEAVKCGIHRTVHAGEVGPSSVVKEAVEVLKAERIGHGYHTTEDPNLYKELLEKNMHFEVCPWSSYLTGACHPDFTKHPATQFRKDKANYSLNTDDPLIFGSTLDVDYSIAAKHMGFTEEEFKRVNINAAKSSFLPESEKKELLYKLYEAYGMILSTGL

SEQ ID NO: 238 - protein, 352 aa, Rattus norvegicus
MAQTPAFNKPKVELHVHLDGAIKPETILYYGKKRGIDLPADTVEGLRNIIGMDKPLSLPDFLAKFDYYMPAIAGCREAIKRIAYEFVEMKAKEGVVYVEVRYSPHLLANSKVDPIPWNQAEGDLTPDEVVDLVNQGLQEGEQAFGIKVRSILCCMRHQPSWSPEVLELCKKYHQKTVVAMDLAGDETIEGSSLFPGHVEAYEGAVKDGIHRTVHAGEVGSAEVVREAVDILKTERVGHGYHTIEDEALYNRLLKENMHFEVCPWSSYLTGAWNPKTTHAVVRFKDDQANYSLNSDDPLIFKSTVDTDYQMVKKDMGFTEEEFKRLNINAAKSSFLPEDEKKELLERLYKEYQ

SEQ ID NO: 239 - protein, 619 aa, Mus musculus
MATAGGSRRAPVPGPRLGLPLAAHLPASLGGEGAKDSVGGEKTSGNNDWFQSSRVPSFAQMLKKNLPVQPSAQTVTLPTGYSSESCSLSNMASKVTQVTGNFPEPLLSKGLSSISNPVLPPKKLPKEFIMKYKRGEINPVSALHQFAQMQRVQLDLKETVTTGNVMGPYFAFCAVVDGIQYKTGLGQNKKESRSNAAKLALDELLQLDEPEPRVLEPAGPPPIPAEPVVTPEAAYVSKVQYEGRQVQYAKISQLVKETFGQLISNHSQYLKCSSSLAAFIIERAGHHEVVAIGTGEYNYSQCIKPNGRVLHDTHAVVTARRSLLRYFYRQLLLFYSKNPAMMEKSIFCTEPASNLLTLKQNINLYLYMNQLPKGSAQIKSQLRLNPHSISAFEANEELSLHVAVEGKIYLTVYCSADGVNRVNSMSSSDKLTRWEVLGVQGALLSHFIQPVYISSILVGDGNCNDTRGLEIAINQRVDDALTSKLPMFYLVNRPHISLVPTAYPLQINLDHKSLSLNWAQGDNSLEIVDGLNGKITESSPFKSGLSMASRLCKAAMLSRFNLLAKEAKTDDLLEARTYHAAKCMSGPYQEAKALLKAYLQQHGYGSWIVKSPCIEQFSM

SEQ ID NO: 240 - protein, 619 aa, Rattus norvegicus
MATAGGSRRAPVPGPRLGLPLAAHLPASLGGEGAKDSLGGEKTSGNNDWFQSSRVPSFAQMLKKNLPVQSSAQTVTLPTGYSSESCSLSNMASKVTQVTGNFPEPLLSKGLSSISNPVLPPKKIPKEFIMKYRRGEINPVSALHQFAQMQRVQLDLKETVTTGNVMGPYFAFCAVVDGIQYKTGLGQNKKESRSNAAKLALDELLQLDEPEPRALEPAGPPPIPAEPIVTPEAAYISKVQYEGRQVQYAKISQLVKETFSQLISSHSQYLKCSSSLAAFIIERGGHHEVVAIGTGEYNYSQCIKPNGRVLHDTHAVVTARRSLLRYFYRQLLLFYSKNPAMMEKSIFCTEPASNLLTLKQNINLYLYMNQLPKGSAQIKSQLRLNPHSISAFEANEELSLHVAVEGKIYLTVYCSADGVNRVNSMSSSDKLTRWEVLGVQGALLSHFIQPVYISSILVGDGNCNDTRGLEIAINQRVDDALTSKLPMFYLVNRPHISLVPTAYPLQINLDHKSLSLNWAQGDNSLEIVDGLSGKITESSPFKSGLSMASRLCKAAMLSRFNLLAKEAKTDDLLEARTYHAAKCLSGPYQEAKALLKAYLQQHGYGSWIVKSPCIEQFSM

SEQ ID NO: 241 - protein, 586 aa, Xenopus laevis
MASNRNWSQHSSVPSFAQMLKKNLPDPGTSPAVNQTSTLSTCCLYNQPDCGTARVTRITGNFPEPFLSKMIVPPSLSSLTPRKVTKEFMVKYRRGDLNPISALYQFAQMQRMEIELKETVTTGNVFGAYFAFCAVVDGLEYKTGMGQNKKEAKANAAKLALDELLLHEDPALIDSENQSLNVIENPPPLPMNPRGTTETSTISRTRTDKRTFIHEKISSIIKETFTNLVSKYPEYENCGSSLAAFVIEKGGQHWEVVAIGTGEFNYGQSLQSDGRVLHDSHAMVVARRSLLRYFYRQLLLLYSGNNGMMDKSIFCTEPATNLLALKPNLNIFLYMNQLPKGAAQTNPQLCLSPHSLSAHEANDKLSLHVSVEGKNIPASYYSGEIVHNLYSMSSTDKLTRWEVLGVQGALLSIFIQPVYINSIIIGNAACSDTRGLEIAVKQRIDDALTSRLPMFYLVNRPYMSIVSSTHLTNNDTANKTLSLNWSQGDACVEVVDAAIGRTVEGSPFKSGSCLASRLCKAAMLCRFNLVVKESKRNAIPSGLSYHEAKRLAGPYQEAKCLLNSYFKQQGFGSWIAKPPIIGEFTM

SEQ ID NO: 242 - protein, 576 aa, Homo sapiens
MASNNHWFQSSQVPSFAQMLKKNLPVQPATKTITTPTGWSSESYGLSKMASKVTQVTGNFPEPLLSKNLSSISNPVLPPKKIPKEFIMKYKRGEINPVSALHQFAQMQRVQLDLKETVTTGNVMGPYFAFCAVVDGIQYKTGLGQNKKESRSNAAKLALDELLQLDEPEPRILETSGPPPFPAEPVVLSELAYVSKVHYEGRHIQYAKISQIVKERFNQLISNRSEYLKYSSSLAAFIIERAGQHEVVAIGTGEYNYSQDIKPDGRVLHDTHAVVTARRSLLRYFYRQLLLFYSKNPAMMEKSIFCTEPTSNLLTLKQNINICLYMNQLPKGSAQIKSQLRLNPHSISAFEANEELCLHVAVEGKIYLTVYCPKDGVNRISSMSSSDKLTRWEVLGVQGALLSHFIQPVYISSILIGDGNCSDTRGLEIAIKQRVDDALTSKLPMFYLVNRPHISLVPSAYPLQMNLEYKFLSLNWAQGDVSLEIVDGLSGKITESSPFKSGMSMASRLCKAAMLSRFNLLAKEAKKELLEAGTYHAAKCMSASYQEAKCKLKSYLQQHGYGSWIVKSPCIEQFNM

SEQ ID NO: 243 - protein, 351 aa, Bos taurus
MMEAEEQPWKTTFYSKLPKVELHAHLNGSISSNTIRKLIAKKPDLKIHDQMTMIDKGEKRTLEECLQMFQIIHLLTTTPEDVLMVTKDVIKEFADDGVKYLELRSTPRGEDATGMTKKTYVESILEGIKQSKEENVDIDVRYLISIDRRGGSSAAKEAVKLAEEFFLSAEDTVLGLDLSGDPSAGQAKDFLEPLLEAKKSGLKLALHLSEIPNQKTETQVLLNLFPDRIGHGTFLSSSEEGSPDLVDFVRQHQIPLELCLTSNVKSQTVPAYDQHHFGFWYSVAHPAVICTDDKGVFATRLSQEYQLVAETFHLTQSQVWDLSYESISYIFASDSTKADLRKKWSHLKPHF

SEQ ID NO: 244 - protein, 348 aa, Danio rerio
MDTEADLFYRQLPKVELHAHLNGSVSFETMEKLIKRKPHLNIEHSMTAIRRGQRRTLDECFQVFKVIHQLVDSEEDILMVAKSVIQEFAADGVKYLELRSTPREVTETGLSKQRYIETVLEAIRQCKQEGVDIDVRFLVAVDRRHGPEVAMQTVKLAEDFLLSSDGTVVGLDLSGDPTVGHGKDLLAALQKAKNCGLKLALHLSEVPSQIDETELLLNLPPDRIGHGTFLHPDVGGSDSLVDKVCKQNIPIEICLTSNVKGQTVPSYDKHHFKYWYNRGHPCVLCTDDKGVFCTDLSQEYQLAASTFGLTKEAVWILSQQAIGYTFAPEPIKQRLEKTWAELKQQILQ

SEQ ID NO: 245 - protein, 347 aa, Xenopus laevis
MAGEGALQFYRDLPKVELHAHLNGSISTATMKKLMARKPHLDIQHGMTMIDKGQKRTLEECFQMFKIIHQITDTAEDILLVTKDVIKEFAADGVKYLELRSTPRDTPAGLTKQAYVETVLEGIKQCKEEGVDIDVRFLLAIDRRGGPTAAKETVKLAEDFFCSSNELVLGLDLSGDPTVGHGRDFMEPLNKARQSGLKLALHLSEIPSQTEETELLLGLPPDRIGHGTFLTTSAHIVEIVKKQHIPLELCITSNIKGQTVSSYNEHHFGFWYNLHHPFVLCTDDKGVFATDLSVEYEIAAKTFNLTPHHVWDLSYQAIDYTFASADVKANLKEKWLLLKPDVFRHAL

SEQ ID NO: 246 - protein, 355 aa, Homo sapiens
MIEAEEQQPCKTDFYSELPKVELHAHLNGSISSHTMKKLIAQKPDLKIHDQMTVIDKGKKRTLEECFQMFQTIHQLTSSPEDILMVTKDVIKEFADDGVKYLELRSTPRRENATGMTKKTYVESILEGIKQSKQENLDIDVRYLIAVDRRGGPLVAKETVKLAEEFFLSTEGTVLGLDLSGDPTVGQAKDFLEPLLEAKKAGLKLALHLSEIPNQKKETQILLDLLPDRIGHGTFLNSGEGGSLDLVDFVRQHRIPLELCLTSNVKSQTVPSYDQHHFGFWYSIAHPSVICTDDKGVFATHLSQEYQLAAETFNLTQSQVWDLSYESINYIFASDSTRSELRKKWNHLKPRVLHI

SEQ ID NO: 247 - protein, 388 aa, Caenorhabditis elegans
MPNNSKHKKKQQRRQQEAQKKSRAKQIETDKKNDEFLDTELDEVSPLVIDDDMTEFKNMPKVELHAHLSGSLSPETIKLIMESDETRAEEIMKKYKLEKPENMTGVFDCFPVIHAILRKPEAIRIAIRQTIKEFEEDNCVYLELRTSPKETDFMTYEDYLQVCIESFEAAKHEFPRIKTFLIVSLDRRMPFETAAHILGLIGEAQQRTNVIVGVELSGDPHLDGRRLLKLFVAARRFHGLGITIHLAEVLQNMADVEDYLNLRPDRIGHGTFLHTDPYTEYLTNKYKIPLEICLSSNVYSKTTTNYRNSHFNYWRKRGVPVFICTDDKGVIPGATLTEEYYKAAITFDLSTEELIGINQDALLNSFAYKYNVTDLTETFRKINNNVLD

SEQ ID NO: 248 - protein, 360 aa, Mus musculus
MEAGQQWPGKTDFYLQLPKVELHAHLNGSISSSTMKKLIAKKPHLNVHGHMTMIDKGKKRTLQECFQMFQVIHQLTTSAEDILMVTKDVIKEFADDGVKYLELRSTPREENATGMTRKTYVESVLEGIKQCKQENLDIDVRYLMAIDRRGGPTIARETVELAKEFFLSTENTVLGLDLSGDPTIGQANDFLEPLLEAKKAGLKLALHLAEIPNREKENQMLLSLLPDRIGHGTFLSASEAGALDQVDFVRQHQIPLELCLTSNIKSQTVPSYDQHHFGFWYSIAHPSVICTDDKGVFATYLSQEYQLAAETFNLTPFQVWDLSYESINYIFACDNTRSELRKRWTHLKQKVLNCNEVNYF

SEQ ID NO: 249 - protein, 583 aa, Homo sapiens
MASASQGADDDGSRRKPRLAASLQISPQPRPWRPLPAQAQSAWGPAPAPATYRAEGGWPQVSVLRDSGPGAGAGVGELGAARAWENLGEQMGKAPRVPVPPAGLSLPLKDPPASQAVSLLTEYAASLGIFLLFREDQPPGPCFPFSVSAELDGVVCPAGTANSKTEAKQQAALSALCYIRSQLENPESPQTSSRPPLAPLSVENILTHEQRCAALVSAGFDLLLDERSPYWACKGTVAGVILEREIPRARGHVKEIYKLVALGTGSSCCAGWLEFSGQQLHDCHGLVIARRALLRFLFRQLLLATQGGPKGKEQSVLAPQPGPGPPFTLKPRVFLHLYISNTPKGAARDIYLPPTSEGGLPHSPPMRLQAHVLGQLKPVCYVAPSLCDTHVGCLSASDKLARWAVLGLGGALLAHLVSPLYSTSLILADSCHDPPTLSRAIHTRPCLDSVLGPCLPPPYVRTALHLFAGPPVAPSEPTPDTCRGLSLNWSLGDPGIEVVDVATGRVKANAALGPPSRLCKASFLRAFHQAARAVGKPYLLALKTYEAAKAGPYQEARRQLSLLLDQQGLGAWPSKPLVGKFRN

SEQ ID NO: 250 - protein, 561 aa, Mus musculus
MASVDEGGRRRPRLAASLQISPGPWKPSGGQEPTEAGDAAPRTAEHGVAGAQEAHREACKALGGSVLSPGPAGDFPGALHGLSMLPKDPPPAQAVALLTQCMANLGVSLTFLEDQTAGPGSSFSVCADLDGLVCPAGTGSSKLEAKQQAALSALQYIQKQLERPEPLVTPRQPLLTSLSIETILTHEQRCAAVVSAGLDRLLSESSPYQACKGTVAAVILEREVQGSIGHSKETYELVALGTGSSSCAGWLEFSGRRLHDCHGLVIARRALLRFFFRQLLLVTQGGPKGQERSVLTPQPGPGPPFALKPGVFLHLYVSNTPKGAAHDIYLPLASEDSVLHSPAFRLQAHVCGQLKPVSYVAPALRDTHVGCLSASDKLARWAILGLGGGLLAHFLPPLYATSLVLADPCHDPSTLNRVIHSRPRLDSVLGSCLPCPYVRTTLHLFAGPLVAPSDPGPSTCHSLSLNWSLGDPDIEVVDVATGRVKTDSSVGPPSRLCKAAFLSAFRQVARALEKPQLLSLQTYEAAKAVPYREARQQLSLLLDQQGLGAWPSKPLVGKFRH

SEQ ID NO: 251 - protein, 580 aa, Macaca fascicularis
MASASQGADDGSRRKPRLAASLQISPEPRPWRPLPPQAQGAWEPAPAMDHAEGGQPQVSVLRDSGPGAGAGVGELGAAQAWENLGEQMGRTPRVPVPPAGLSLPLKDPPASQAVSLLTEYAASLGIILLFREDQQPGPCFPFSVSAELDGVVCPAGTANSKTEAKQQAALSALCYIRSQLESPESPQTSSRPPPPPLSVDSILTHGQRCAALVSAGFDLLLDERSPYWACKGTVAGVILEREIPGARGHVKEIYKLVALGTGSSCCAGWLEFSGQQLHDCHGLVIARRALLRFLFRQLLLATQGGAKGKEQSVLAPQPGPGPPFTLKPRVFLHLYISNTPKGAARDIYLPPTSEGGLPHSPPMRLQAHVLGQLKPVCYVAPSLCDTHVGCLSASDKLARWAVLGLGGALLAHLVSPLYSTSLILADSCHDPPTLSRAIHTRPCLDSVLGPCLPPPYVRTALHLFSGPPVAPSEPTPDTCHGLSLNWSLGDPGIEVVDVATGRVKANAALGPPSRLCKASFLRAFHQVARAVGKPYLLALKTYEAAKAGPYQEARRQLSLLLDQQGLGAWPSKPLVGKFRN

SEQ ID NO: 252 - protein, 193 aa, Felis catus
MNPLQEVIFCRQFGNQHRVPKPYYRRKTYLCYQLKLPEGTLIHKDCLRNKKKRHAEMCFIDKIKALTRDTSQRFEIICYITWSPCPFCAEELVAFVKDNPHLSLRIFASRLYVHWRWKYQQGLRHLHASGIPVAVMSLPEFEDCWRNFVDHQDRSFQPWPNLDQYSKSIKRRLGKILTPLNDLRNDFRNLKLE

SEQ ID NO: 253 - protein, 199 aa, Homo sapiens
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 254 - protein, 396 aa, Cricetulus longicaudatus
MGPFCLGCSHRKCYSPIRNLISQETFKFHFKNLGYAKGRKDTFLCYEVTRKDCDSPVSLHHGVFKNKGNIHAEVCFLYWFHDKVLKVLSPREEFKITWYMSWSPCFECAEQIVRFLATHHYLSLDIFSSRLYNVQDPETQQNLCRLVQEGAQVAAMDLYEFKKCWKKFVTMVAGDSGLGKRLLTNFRYQDSKLQEILRRMDPLSEEEFYSQFYNQRVKHLCYYHRMKPYLCYQLEQFNGQAPLKGCLLSEKGKQHAEILFLDKIRSMELSQVTITCYLTWSPCPNCAWRLAAFKRDRPDLILHIYTSRLYFHWKRPFQKGLCSLWQSGILVDVMDLPQFTDCWTNFVNPKRPFWPWKGLEIISRRTQRRLRRIKESWGLQDLVNDFGNLQLGPPMS

SEQ ID NO: 255 - protein, 429 aa, Rattus norvegicus
MGPFCLGCSHRKCYSPIRNLISQETFKFHFKNLRYAIDRKDTFLCYEVTRKDCDSPVSLHHGVFKNKDNIHAEICFLYWFHDKVLKVLSPREEFKITWYMSWSPCFECAEQVLRFLATHHNLSLDIFSSRLYNIRDPENQQNLCRLVQEGAQVAAMDLYEFKKCWKKFVDNGGRRFRPWKKLLTNFRYQDSKLQEILRPCYIPVPSSSSSTLSNICLTKGLPETRFCVERRRVHLLSEEEFYSQFYNQRVKHLCYYHGVKPYLCYQLEQFNGQAPLKGCLLSEKGKQHAEILFLDKIRSMELSQVIITCYLTWSPCPNCAWQLAAFKRDRPDLILHIYTSRLYFHWKRPFQKGLCSLWQSGILVDVMDLPQFTDCWTNFVNPKRPFWPWKGLEIISRRTQRRLHRIKESWGLQDLVNDFGNLQLGPPMS

SEQ ID NO: 256 - protein, 211 aa, Pongo pygmaeus
MALLTAKTFSLQFNNKRRIKRPYYPRKALLCYQLTPQNGSTPTRGYFKNKKKCHAEIRFINEIKSMGLDETQCYQVTCYLTWSPCPSCVRELVAFIKAHDHLNLRIFASRLYCHWCRRQQEGLRLLCGSQVPVEVMGSREFADCWENFVDHEKPLSFNPSEMLEELDKNSRAIKRRLERIKQSWSVDVLENGLRSLQLGPVSSSLSRSNSR

SEQ ID NO: 257 - protein, 192 aa, Felis catus
MEPWRPSPRNPMDRIDPNTFRFHFPNLLYASGRKLCYLCFQVETEDYFSCDDSDRGVFRNKVHPWARCHAEQCFLSWFRDQYPYRDEYYNVTWFLSWSPCPTCAEEVVEFLEEYRNLTLSIFTSRLYYFWDPNYQEGLCKLWDAGVQLDIMSCDDFKHCWDNFVDHKGMRFQRRNLLKDYDFLAAELQEILR

SEQ ID NO: 258 - protein, 200 aa, Homo sapiens
MALLTAETFRLQFNNKRRLRRPYYPRKALLCYQLTPQNGSTPTRGYFENKKKCHAEICFINEIKSMGLDETQCYQVTCYLTWSPCSSCAWELVDFIKAHDHLNLGIFASRLYYHWCKPQQKGLRLLCGSQVPVEVMGFPEFADCWENFVDHEKPLSFNPYKMLEELDKNSRAIKRRLERIKIPGVRAQGRYMDILCDAEV

SEQ ID NO: 259 - protein, 370 aa, Macaca mulatta
MVEPMDPRTFVSNFNNRPILSGLNTVWLCCEVKTKDPSGPPLDAKIFQGKVYSKAKYHPEMRFLRWFHKWRQLHHDQEYKVTWYVSWSPCTRCANSVATFLAKDPKVTLTIFVARLYYFWKPDYQQALRILCQKRGGPHATMKIMNYNEFQDCWNKFVDGRGKPFKPRNNLPKHYTLLQATLGELLRHLMDPGTFTSNFNNKPWVSGQHETYLCYKVERLHNDTWVPLNQHRGFLRNQAPNIHGFPKGRHAELCFLDLIPFWKLDGQQYRVTCFTSWSPCFSCAQEMAKFISNNEHVSLCIFAARIYDDQGRYQEGLRALHRDGAKIAMMNYSEFEYCWDTFVDRQGRPFQPWDGLDEHSQALSGRLRAI

SEQ ID NO: 260 - protein, 384 aa, Pan troglodytes
MKPHFRNPVERMYQDTFSDNFYNRPILSHRNTVWLCYEVKTKGPSRPPLDAKIFRGQVYSKLKYHPEMRFFHWFSKWRKLHRDQEYEVTWYISWSPCTKCTRDVATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTSNFNNELWVRGRHETYLCYEVERLHNDTWVLLNQRRGFLCNQAPHKHGFLEGRHAELCFLDVIPFWKLDLHQDYRVTCFTSWSPCFSCAQEMAKFISNNKHVSLCIFAARIYDDQGRCQEGLRTLAKAGAKISIMTYSEFKHCWDTFVDHQGCPFQPWDGLEEHSQALSGRLRAILQNQGN

SEQ ID NO: 261 - protein, 377 aa, Chlorocebus aethiops
MNPQIRNMVEQMEPDIFVYYFNNRPILSGRNTVWLCYEVKTKDPSGPPLDANIFQGKLYPEAKDHPEMKFLHWFRKWRQLHRDQEYEVTWYVSWSPCTRCANSVATFLAEDPKVTLTIFVARLYYFWKPDYQQALRILCQERGGPHATMKIMNYNEFQHCWNEFVDGQGKPFKPRKNLPKHYTLLHATLGELLRHVMDPGTFTSNFNNKPWVSGQRETYLCYKVERSHNDTWVLLNQHRGFLRNQAPDRHGFPKGRHAELCFLDLIPFWKLDDQQYRVTCFTSWSPCFSCAQKMAKFISNNKHVSLCIFAARIYDDQGRCQEGLRTLHRDGAKIAVMNYSEFEYCWDTFVDRQGRPFQPWDGLDEHSQALSGRLRAI

SEQ ID NO: 262 - protein, 373 aa, Homo sapiens
MKPHFRNTVERMYRDTFSYNFYNRPILSRRNTVWLCYEVKTKGPSRPRLDAKIFRGQVYSQPEHHAEMCFLSWFCGNQLPAYKCFQITWFVSWTPCPDCVAKLAEFLAEHPNVTLTISAARLYYYWERDYRRALCRLSQAGARVKIMDDEEFAYCWENFVYSEGQPFMPWYKFDDNYAFLHRTLKEILRNPMEAMYPHIFYFHFKNLRKAYGRNESWLCFTMEVVKHHSPVSWKRGVFRNQVDPETHCHAERCFLSWFCDDILSPNTNYEVTWYTSWSPCPECAGEVAEFLARHSNVNLTIFTARLYYFWDTDYQEGLRSLSQEGASVEIMGYKDFKYCWENFVYNDDEPFKPWKGLKYNFLFLDSKLQEILE

SEQ ID NO: 263 - protein, 384 aa, Homo sapiens
MKPHFRNTVERMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQNQEN

SEQ ID NO: 264 - protein, 190 aa, Homo sapiens
MNPQIRNPMKAMYPGTFYFQFKNLWEANDRNETWLCFTVEGIKRRSVVSWKTGVFRNQVDSETHCHAERCFLSWFCDDILSPNTKYQVTWYTSWSPCPDCAGEVAEFLARHSNVNLTIFTARLYYFQYPCYQEGLRSLSQEGVAVEIMDYEDFKYCWENFVYNDNEPFKPWKGLKTNFRLLKRRLRESLQ

SEQ ID NO: 265 - protein, 382 aa, Homo sapiens
MNPQIRNPMERMYRDTFYDNFENEPILYGRSYTWLCYEVKIKRGRSNLLWDTGVFRGQVYFKPQYHAEMCFLSWFCGNQLPAYKCFQITWFVSWTPCPDCVAKLAEFLSEHPNVTLTISAARLYYYWERDYRRALCRLSQAGARVTIMDYEEFAYCWENFVYNEGQQFMPWYKFDENYAFLHRTLKEILRYLMDPDTFTFNFNNDPLVLRRRQTYLCYEVERLDNGTWVLMDQHMGFLCNEAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFEYCWDTFVYRQGCPFQPWDGLEEHSQALSGRLRAILQNQGN

SEQ ID NO: 266 - protein, 210 aa, Macaca mulatta
MALLTAKTFSLQFNNKRRVNKPYYPRKALLCYQLTPQNGSTPTRGHLKNKKKDHAEIRFINKIKSMGLDETQCYQVTCYLTWSPCPSCAGELVDFIKAHRHLNLRIFASRLYYHWRPNYQEGLLLLCGSQVPVEVMGLPEFTDCWENFVDHKEPPSFNPSEKLEELDKNSQAIKRRLERIKSRSVDVLENGLRSLQLGPVTPSSSIRNSR

SEQ ID NO: 267 - protein, 386 aa, Homo sapiens
MNPQIRNPMERMYRDTFYDNFENEPILYGRSYTWLCYEVKIKRGRSNLLWDTGVFRGPVLPKRQSNHRQEVYFRFENHAEMCFLSWFCGNRLPANRRFQITWFVSWNPCLPCVVKVTKFLAEHPNVTLTISAARLYYYRDRDWRWVLLRLHKAGARVKIMDYEDFAYCWENFVCNEGQPFMPWYKFDDNYASLHRTLKEILRNPMEAMYPHIFYFHFKNLLKACGRNESWLCFTMEVTKHHSAVFRKRGVFRNQVDPETHCHAERCFLSWFCDDILSPNTNYEVTWYTSWSPCPECAGEVAEFLARHSNVNLTIFTARLCYFWDTDYQEGLCSLSQEGASVKIMGYKDFVSCWKNFVYSDDEPFKPWKGLQTNFRLLKRRLREILQ

SEQ ID NO: 268 - protein, 440 aa, Mus musculus
MQPQRLGPRAGMGPFCLGCSHRKCYSPIRNLISQETFKFHFKNLGYAKGRKDTFLCYEVTRKDCDSPVSLHHGVFKNKDNIHAEICFLYWFHDKVLKVLSPREEFKITWYMSWSPCFECAEQIVRFLATHHNLSLDIFSSRLYNVQDPETQQNLCRLVQEGAQVAAMDLYEFKKCWKKFVDNGGRRFRPWKRLLTNFRYQDSKLQEILRPCYISVPSSSSSTLSNICLTKGLPETRFWVEGRRMDPLSEEEFYSQFYNQRVKHLCYYHRMKPYLCYQLEQFNGQAPLKGCLLSEKGKQHAEILFLDKIRSMELSQVTITCYLTWSPCPNCAWQLAAFKRDRPDLILHIYTSRLYFHWKRPFQKGLCSLWQSGILVDVMDLPQFTDCWTNFVNPKRPFWPWKGLEIISRRTQRRLRRIKESWGLQDLVNDFGNLQLGPPMS

SEQ ID NO: 269 - protein, 229 aa, Rattus norvegicus [sequence omitted]

SEQ ID NO: 270 - protein, 236 aa, Homo sapiens
MTSEKGPSTGDPTLRRRIEPWEFDVFYDPRELRKEACLLYEIKWGMSRKIWRSSGKNTTNHVEVNFIKKFTSERDFHPSMSCSITWFLSWSPCWECSQAIREFLSRHPGVTLVIYVARLFWHMDQQNRQGLRDLVNSGVTIQIMRASEYYHCWRNFVNYPPGDEAHWPQYPPLWMMLYALELHCIILSLPPCLKISRRWQNHLTFFRLHLQNCHYQTIPPHILLATGLIHPSVAWR

SEQ ID NO: 271 - protein, 236 aa, Oryctolagus cuniculus
MASEKGPSNKDYTLRRRIEPWEFEVFFDPQELRKEACLLYEIKWGASSKTWRSSGKNTTNHVEVNFLEKLTSEGRLGPSTCCSITWFLSWSPCWECSMAIREFLSQHPGVTLIIFVARLFQHMDRRNRQGLKDLVTSGVTVRVMSVSEYCYCWENFVNYPPGKAAQWPRYPPRWMLMYALELYCIILGLPPCLKISRRHQKQLTFFSLTPQYCHYKMIPPYILLATGLLQPSVPWR

SEQ ID NO: 272 - protein, 229 aa, Mus musculus
MSSETGPVAVDPTLRRRIEPHEFEVFFDPRELRKETCLLYEINWGGRHSVWRHTSQNTSNHVEVNFLEKFTTERYFRPNTRCSITWFLSWSPCGECSRAITEFLSRHPYVTLFIYIARLYHHTDQRNRQGLRDLISSGVTIQIMTEQEYCYCWRNFVNYPPSNEAYWPRYPHLWVKLYVLELYCIILGLPPCLKILRRKQPQLTFFTITLQTCHYQRIPPHLLWATGLK

SEQ ID NO: 273 - protein, 229 aa, Mesocricetus auratus
MSSETGPVVVDPTLRRRIEPHEFDAFFDQGELRKETCLLYEIRWGGRHNIWRHTGQNTSRHVEINFIEKFTSERYFYPSTRCSIVWFLSWSPCGECSKAITEFLSGHPNVTLFIYAARLYHHTDQRNRQGLRDLISRGVTIRIMTEQEYCYCWRNFVNYPPSNEVYWPRYPNLWMRLYALELYCIHLGLPPCLKIKRRHQYPLTFFRLNLQSCHYQRIPPHILWATGFI

SEQ ID NO: 274 - protein, 235 aa, Monodelphis domestica
MNSKTGPSVGDATLRRRIKPWEFVAFFNPQELRKETCLLYEIKWGNQNIWRHSNQNTSQHAEINFMEKFTAERHFNSSVRCSITWFLSWSPCWECSKAIRKFLDHYPNVTLAIFISRLYWHMDQQHRQGLKELVHSGVTIQIMSYSEYHYCWRNFVDYPQGEEDYWPKYPYLWIMLYVLELHCIILGLPPCLKISGSHSNQLALFSLDLQDCHYQKIPYNVLVATGLVQPFVTWR

SEQ ID NO: 275 - protein, 236 aa, Pongo pygmaeus
MTSEKGPSTGDPTLRRRIESWEFDVFYDPRELRKETCLLYEIKWGMSRKIWRSSGKNTTNHVEVNFIKKFTSERRFHSSISCSITWFLSWSPCWECSQAIREFLSQHPGVTLVIYVARLFWHMDQRNRQGLRDLVNSGVTIQIMRASEYYHCWRNFVNYPPGDEAHWPQYPPLWMMLYALELHCIILSLPPCLKISRRWQNHLAFFRLHLQNCHYQTIPPHILLATGLIHPSVTWR

SEQ ID NO: 276 - protein, 224 aa, Bos taurus
MAQKEEAAAAAEPASQNGEEVENLEDPEKLKELIELPPFEIVTGERLPAHYFKFQFRNVEYSSGRNKTFLCYVVEAQSKGGQVQASRGYLEDEHATNHAEEAFFNSIMPTFDPALRYMVTWYVSSSPCAACADRIVKTLNKTKNLRLLILVGRLFMWEEPEIQAALRKLKEAGCRLRIMKPQDFEYIWQNFVEQEEGESKAFEPWEDIQENFLYYEEKLADILK

SEQ ID NO: 277 - protein, 224 aa, Mus musculus
MAQKEEAAEAAAPASQNGDDLENLEDPEKLKELIDLPPFEIVTGVRLPVNFFKFQFRNVEYSSGRNKTFLCYVVEVQSKGGQAQATQGYLEDEHAGAHAEEAFFNTILPAFDPALKYNVTWYVSSSPCAACADRILKTLSKTKNLRLLILVSRLFMWEEPEVQAALKKLKEAGCKLRIMKPQDFEYIWQNFVEQEEGESKAFEPWEDIQENFLYYEEKLADILK

SEQ ID NO: 278 - protein, 224 aa, Homo sapiens
MAQKEEAAVATEAASQNGEDLENLDDPEKLKELIELPPFEIVTGERLPANFFKFQFRNVEYSSGRNKTFLCYVVEAQGKGGQVQASRGYLEDEHAAAHAEEAFFNTILPAFDPALRYNVTWYVSSSPCAACADRIIKTLSKTKNLRLLILVGRLFMWEEPEIQAALKKLKEAGCKLRIMKPQDFEYVWQNFVEQEEGESKAFQPWEDIQENFLYYEEKLADILK

SEQ ID NO: 279 - protein, 224 aa, Pongo pygmaeus
MAQKEEAAAATEAASQNGEDLENLDDPEKLKELIELPPFEIVTGERLPANFFKFQFRNVEYSSGRNKTFLCYVVEAQGKGGQVQASRGYLEDEHAAAHAEEAFFNTILPAFDPALRYNVTWYVSSSPCAACADRIIKTLSKTKNLRLLILVGRLFMWEELEIQDALKKLKEAGCKLRIMKPQDFEYVWQNFVEQEEGESKAFQPWEDIQENFLYYEEKLADILK

SEQ ID NO: 280 - protein, 367 aa, Homo sapiens
MEPIYEEYLANHGTIVKPYYWLSFSLDCSNCPYHIRTGEEARVSLTEFCQIFGFPYGTTFPQTKHLTFYELKTSSGSLVQKGHASSCTGNYIHPESMLFEMNGYLDSAIYNNDSIRHIILYSNNSPCNEANHCCISKMYNFLITYPGITLSIYFSQLYHTEMDFPASAWNREALRSLASLWPRVVLSPISGGIWHSVLHSFISGVSGSHVFQPILTGRALADRHNAYEINAITGVKPYFTDVLLQTKRNPNTKAQEALESYPLNNAFPGQFFQMPSGQLQPNLPPDLRAPVVFVLVPLRDLPPMHMGQNPNKPRNIVRHLNMPQMSFQETKDLGRLPTGRSVEIVEITEQFASSKEADEKKKKKGKK

SEQ ID NO: 281 - protein, 388 aa, Rattus norvegicus
MEPLYEEYLTHSGTIVKPYYWLSVSLNCTNCPYHIRTGEEARVPYTEFHQTFGFPWSTYPQTKHLTFYELRSSSGNLIQKGLASNCTGSHTHPESMLFERDGYLDSLIFHDSNIRHIILYSNNSPCDEANHCCISKMYNFLMNYPEVTLSVFFSQLYHTENQFPTSAWNREALRGLASLWPQVTLSAISGGIWQSILETFVSGISEGLTAVRPFTAGRTLTDRYNAYEINCITEVKPYFTDALHSWQKENQDQKVWAASENQPLHNTTPAQWQPDMSQDCRTPAVFMLVPYRDLPPIHVNPSPQKPRTVVRHLNTLQLSASKVKALRKSPSGRPVKKEEARKGSTRSQEANETNKSKWKKQTLFIKSNICHLLEREQKKIGILSSWSV

SEQ ID NO: 282 - protein, 374 aa, Mus musculus
MEPLYEEILTQGGTIVKPYYWLSLSLGCTNCPYHIRTGEEARVPYTEFHQTFGFPWSTYPQTKHLTFYELRSSSKNLIQKGLASNCTGSHNHPEAMLFEKNGYLDAVIFHNSNIRHIILYSNNSPCNEAKHCCISKMYNFLMNYPEVTLSVFFSQLYHTEKQFPTSAWNRKALQSLASLWPQVTLSPICGGLWHAILEKFVSNISGSTVPQPFIAGRILADRYNTYEINSIIAAKPYFTDGLLSRQKENQNREAWAAFEKHPLGSAAPAQRQPTRGQDPRTPAVLMLVSNRDLPPIHVGSTPQKPRTVVRHLNMLQLSSFKVKDVKKPPSGRPVEEVEVMKESARSQKANKKNRSQWKKQTLVIKPRICRLLER

SEQ ID NO: 283 - protein, 363 aa, Macaca fascicularis
MEPTYEEYLANHGTIVKPYYWLSFSLDCSNCPYHIRTGEEARVSLTEFCQIFGFPYGTTYPQTKHLTFYELKTSSGSLVQKGHASSCTGNYIHPESMLFEMNGYLDSAIYNNDSIRHIILYCNNSPCNEANHCCISKVYNFLITYPGITLSIYFSQLYHTEMDFPASAWNREALRSLASLWPRVVLSPISGGIWHSVLHSFVSGVSGSHVFQPILTGRALTDRYNAYEINAITGVKPFFTDVLLHTKRNPNTKAQMALESYPLNNAFPGQSFQMTSGIPPDLRAPVVFVLLPLRDLPPMHMGQDPNKPRNIIRHLNMPQMSFQETKDLERLPTRRSVETVEITERFASSKQAEEKTKKKKGKK

SEQ ID NO: 284 - protein, 198 aa, Homo sapiens [sequence omitted]

SEQ ID NO: 285 - protein, 198 aa, Mus musculus
MDSLLMKQKKFLYHFKNVRWAKGRHETYLCYVVKRRDSATSCSLDFGHLRNKSGCHVELLFLRYISDWDLDPGRCYRVTWFTSWSPCYDCARHVAEFLRWNPNLSLRIFTARLYFCEDRKAEPEGLRRLHRAGVQIGIMTFKDYFYCWNTFVENRERTFKAWEGLHENSVRLTRQLRRILLPLYEVDDLRDAFRMLGF

SEQ ID NO: 286 - protein, 198 aa, Canis sp.
  source 1..198: note = Canis lupus familiaris
MDSLLMKQRKFLYHFKNVRWAKGRHETYLCYVVKRRDSATSFSLDFGHLRNKSGCHVELLFLRYISDWDLDPGRCYRVTWFTSWSPCYDCARHVADFLRGYPNLSLRIFAARLYFCEDRKAEPEGLRRLHRAGVQIAIMTFKDYFYCWNTFVENREKTFKAWEGLHENSVRLSRQLRRILLPLYEVDDLRDAFRTLGL

SEQ ID NO: 287 - protein, 199 aa, Bos taurus
MDSLLKKQRQFLYQFKNVRWAKGRHETYLCYVVKRRDSPTSFSLDFGHLRNKAGCHVELLFLRYISDWDLDPGRCYRVTWFTSWSPCYDCARHVADFLRGYPNLSLRIFTARLYFCDKERKAEPEGLRRLHRAGVQIAIMTFKDYFYCWNTFVENHERTFKAWEGLHENSVRLSRQLRRILLPLYEVDDLRDAFRTLGL

SEQ ID NO: 288 - RNA, 66 nt, synthetic construct
  source 1..66: mol_type = other RNA
nnnnnnnnnngtttgagagctaggccaacatgaggatcacccatgtctgcagggcctgctgttttg

SEQ ID NO: 289 - RNA, 66 nt, synthetic construct
  source 1..66: mol_type = other RNA
nnnnnnnnnngtttgagagctatgctgggccaacatgaggatcacccatgtctgcagggccttttg

SEQ ID NO: 290 - RNA, 66 nt, synthetic construct
  source 1..66: mol_type = other RNA
nnnnnnnnnngtttgagagctatgctgttttgggccaacatgaggatcacccatgtctgcagggcc

SEQ ID NO: 291 - RNA, 61 nt, synthetic construct
  source 1..61: mol_type = other RNA
nnnnnnnnnnnnnnnnnnnngtttgagagctagggccctgaagaagggccctgctgttttg

SEQ ID NO: 292 - RNA, 61 nt, synthetic construct
  source 1..61: mol_type = other RNA
nnnnnnnnnnnnnnnnnnnngtttgagagctatgctggggccctgaagaagggcccttttg

SEQ ID NO: 293 - RNA, 61 nt, synthetic construct
  source 1..61: mol_type = other RNA
nnnnnnnnnnnnnnnnnnnngtttgagagctatgctgttttggggccctgaagaagggccc

SEQ ID NO: 294 - RNA, 82 nt, synthetic construct
  source 1..82: mol_type = other RNA
nnnnnnnnnnnnnnnnnnnngtttgagagctagggccctgaagaagggcgggccctgaagaagggcccaacctgctgttttg

SEQ ID NO: 295 - RNA, 42 nt, synthetic construct
  source 1..42: mol_type = other RNA
nnnnnnnnnnnnnnnnnnnngtttgagagctatgctgttttg

SEQ ID NO: 296 - RNA, 32 nt, synthetic construct
  source 1..32: mol_type = other RNA
nnnnnnnnnngtttgagagctatgctgttttg

SEQ ID NO: 297 - RNA, 69 nt, synthetic construct
  source 1..69: mol_type = other RNA
nnnnnnnnnngtttgagagctatgctgttttgggccaacatgaggatcacccatgtctgcagggccttt

SEQ ID NO: 298 - RNA, 86 nt, synthetic construct
  source 1..86: mol_type = other RNA
nnnnnnnnnngtttgagagctatgctgttttgggccagcgaggagcaccagccctcgcgtgtgacgatgacgatcacgcatcgttt

SEQ ID NO: 299 - RNA, 89 nt, synthetic construct
  source 1..89: mol_type = other RNA
nnnnnnnnnngtttgagagctatgctgttttgggccaggaatgaccaccaggcattccgatccgacgatggaccatcaggccatcgttt

SEQ ID NO: 300 - RNA, 64 nt, synthetic construct
  source 1..64: mol_type = other RNA
nnnnnnnnnnnnnnnnnnnngtttgagagctatgctgttttggggccctgaagaagggcccttt

SEQ ID NO: 301 - RNA, 83 nt, synthetic construct
  source 1..83: mol_type = other RNA
nnnnnnnnnnnnnnnnnnnngtttgagagctatgctgttttggggccctgaagaagggcccgggccctgaagaagggcccttt

SEQ ID NO: 302 - RNA, 69 nt, Mus musculus
  source 1..69: mol_type = other RNA
  modified_base 1..3: mod_base = OTHER; note = 2'-O-methyl
  modified_base 67..69: mod_base = OTHER; note = 2'-O-methyl
  modified_base 1..4: mod_base = OTHER; note = phosphorothioate linkage
  modified_base 66..69: mod_base = OTHER; note = phosphorothioate linkage
ttcctctgtcgtttgagagctatgctgttttgggccaacatgaggatcacccatgtctgcagggccttt

SEQ ID NO: 303 - RNA, 64 nt, Mus musculus
  source 1..64: mol_type = other RNA
  modified_base 1..3: mod_base = OTHER; note = 2'-O-methyl
  modified_base 62..64: mod_base = OTHER; note = 2'-O-methyl
  modified_base 1..4: mod_base = OTHER; note = phosphorothioate linkage
  modified_base 61..64: mod_base = OTHER; note = phosphorothioate linkage
caggttccatgggatgctctgtttgagagctatgctgttttggggccctgaagaagggcccttt

SEQ ID NO: 304 - RNA, 69 nt, Homo sapiens
  source 1..69: mol_type = other RNA
  modified_base 1..3: mod_base = OTHER; note = 2'-O-methyl
  modified_base 67..69: mod_base = OTHER; note = 2'-O-methyl
  modified_base 1..4: mod_base = OTHER; note = phosphorothioate linkage
  modified_base 66..69: mod_base = OTHER; note = phosphorothioate linkage
ttcatccgccgtttgagagctatgctgttttgggccaacatgaggatcacccatgtctgcagggccttt

SEQ ID NO: 305 - RNA, 64 nt, Homo sapiens
  source 1..64: mol_type = other RNA
  modified_base 1..3: mod_base = OTHER; note = 2'-O-methyl
  modified_base 62..64: mod_base = OTHER; note = 2'-O-methyl
  modified_base 1..4: mod_base = OTHER; note = phosphorothioate linkage
  modified_base 61..64: mod_base = OTHER; note = phosphorothioate linkage
caggttccacgggatgctctgtttgagagctatgctgttttggggccctgaagaagggcccttt

SEQ ID NO: 306 - RNA, 69 nt, Macaca mulatta; identical to SEQ ID NO: 304
  source 1..69: mol_type = other RNA
  modified_base 1..3: mod_base = OTHER; note = 2'-O-methyl
  modified_base 67..69: mod_base = OTHER; note = 2'-O-methyl
  modified_base 1..4: mod_base = OTHER; note = phosphorothioate linkage
  modified_base 66..69: mod_base = OTHER; note = phosphorothioate linkage

SEQ ID NO: 307 - RNA, 64 nt, Macaca mulatta; identical to SEQ ID NO: 303
  source 1..64: mol_type = other RNA
  modified_base 1..3: mod_base = OTHER; note = 2'-O-methyl
  modified_base 62..64: mod_base = OTHER; note = 2'-O-methyl
  modified_base 1..4: mod_base = OTHER; note = phosphorothioate linkage
  modified_base 61..64: mod_base = OTHER; note = phosphorothioate linkage

SEQ ID NO: 308 - protein, 1368 aa, synthetic construct [sequence omitted]

SEQ ID NO: 309 - protein, 1368 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 310 - protein, 1368 aa, synthetic construct; identical to SEQ ID NO: 47

SEQ ID NO: 311 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 312 - protein, 1629 aa, synthetic construct
MNFKILPIAIDLGVKNTGVFSAFYQKGTSLERLDNKNGKVYELSKDSYTLLMNNRTARRHQRRGIDRKQLVKRLFKLIWTEQLNLEWDKDTQQAISFLFNRRGFSFITDGYSPEYLNIVPEQVKAILMDIFDDYNGEDDLDSYLKLATEQESKISEIYNKLMQKILEFKLMKLCTDIKDDKVSTKTLKEITSYEFELLADYLANYSESLKTQKFSYTDKQGNLKELSYYHHDKYNIQEFLKRHATINDRILDTLLTDDLDIWNFNFEKFDFDKNEEKLQNQEDKDHIQAHLHHFVFAVNKIKSEMASGGRHRSQYFQEITNVLDENNHQEGYLKNFCENLHNKKYSNLSVKNLVNLIGNLSNLELKPLRKYFNDKIHAKADHWDEQKFTETYCHWILGEWRVGVKDQDKKDGAKYSYKDLCNELKQKVTKAGLVDFLLELDPCRTIPPYLDNNNRKPPKCQSLILNPKFLDNQYPNWQQYLQELKKLQSIQNYLDSFETDLKVLKSSKDQPYFVEYKSSNQQIASGQRDYKDLDARILQFIFDRVKASDELLLNEIYFQAKKLKQKASSELEKLESSKKLDEVIANSQLSQILKSQHTNGIFEQGTFLHLVCKYYKQRQRARDSRLYIMPEYRYDKKLHKYNNTGRFDDDNQLLTYCNHKPRQKRYQLLNDLAGVLQVSPNFLKDKIGSDDDLFISKWLVEHIRGFKKACEDSLKIQKDNRGLLNHKINIARNTKGKCEKEIFNLICKIEGSEDKKGNYKHGLAYELGVLLFGEPNEASKPEFDRKIKKFNSIYSFAQIQQIAFAERKGNANTCAVCSADNAHRMQQIKITEPVEDNKDKIILSAKAQRLPAIPTRIVDGAVKKMATILAKNIVDDNWQNIKQVLSAKHQLHIPIITESNAFEFEPALADVKGKSLKDRRKKALERISPENIFKDKNNRIKEFAKGISAYSGANLTDGDFDGAKEELDHIIPRSHKKYGTLNDEANLICVTRGDNKNKGNRIFCLRDLADNYKLKQFETTDDLEIEKKIADTIWDANKKDFKFGNYRSFINLTPQEQKAFRHALFLADENPIKQAVIRAINNRNRTFVNGTQRYFAEVLANNIYLRAKKENLNTDKISFDYFGIPTIGNGRGIAEIRQLYEKVDSDIQAYAKGDKPQASYSHLIDAMLAFCIAADEHRNDGSIGLEIDKNYSLYPLDKNTGEVFTKDIFSQIKITDNEFSDKKLVRKKAIEGFNTHRQMTRDGIYAENYLPILIHKELNEVRKGYTWKNSEEIKIFKGKKYDIQQLNNLVYCLKFVDKPISIDIQISTLEELRNILTTNNIAATAEYYYINLKTQKLHEYYIENYNTALGYKKYSKEMEFLRSLAYRSERVKIKSIDDVKQVLDKDSNFIIGKITLPFKKEWQRLYREWQNTTIKDDYEFLKSFFNVKSITKLHKKVRKDFSLPISTNEGKFLVKRKTWDNNFIYQILNDSDSRADGTKPFIPAFDISKNEIVEAIIDSFTSKNIFWLPKNIELQKVDNKNIFAIDTSKWFEVETPSDLRDIGIATIQYKIDNNSRPKVRVKLDYVIDDDSKINYFMNHSLLKSRYPDKVLEILKQSTIIEFESSGFNKTIKEMLGMKLAGIYNETSNN

SEQ ID NO: 313 - protein, 1122 aa, synthetic construct
MGSDLVLGLDIGIGSVGVGILNKVTGEIIHKNSRIFPAAQAENNLVRRTNRQGRRLARRKKHRRVRLNRLFEESGLITDFTKISINLNPYQLRVKGLTDELSNEELFIALKNMVKHRGISYLDDASDDGNSSVGDYAQIVKENSKQLETKTPGQIQLERYQTYGQLRGDFTVEKDGKKHRLINVFPTSAYRSEALRILQTQQEFNPQITDEFINRYLEILTGKRKYYHGPGNEKSRTDYGRYRTSGETLDNIFGILIGKCTFYPDEFRAAKASYTAQEFNLLNDLNNLTVPTETKKLSKEQKNQIINYVKNEKAMGPAKLFKYIAKLLSCDVADIKGYRIDKSGKAEIHTFEAYRKMKTLETLDIEQMDRETLDKLAYVLTLNTEREGIQEALEHEFADGSFSQKQVDELVQFRKANSSIFGKGWHNFSVKLMMELIPELYETSEEQMTILTRLGKQKTTSSSNKTKYIDEKLLTEEIYNPVVAKSVRQAIKIVNAAIKEYGDFDNIVIEMARETNEDDEKKAIQKIQKANKDEKDAAMLKAANQYNGKAELPHSVFHGHKQLATKIRLWHQQGERCLYTGKTISIHDLINNSNQFEVDHILPLSITFDDSLANKVLVYATANQEKGQRTPYQALDSMDDAWSFRELKAFVRESKTLSNKKKEYLLTEEDISKFDVRKKFIERNLVDTRYASRVVLNALQEHFRAHKIDTKVSVVRGQFTSQLRRHWGIEKTRDTYHHHAVDALIIAASSQLNLWKKQKNTLVSYSEDQLLDIETGELISDDEYKESVFKAPYQHFVDTLKSKEFEDSILFSYQVDSKFNRKISDATIYATRQAKVGKDKADETYVLGKIKDIYTQDGYDAFMKIYKKDKSKFLMYRHDPQTFEKVIEPILENYPNKQINEKGKEVPCNPFLKYKEEHGYIRKYSKKGNGPEIKSLKYYDSKLGNHIDITPKDSNNKVVLQSVSPWRADVYFNKTTGKYEILGLKYADLQFEKGTGTYKISQEKYNDIKKKEGVDSDSEFKFTLYKNDLLLVKDTETKEQQLFRFLSRTMPKQKHYVELKPYDKQKFEGGEALIKVLGNVANSGQCKKGLGKSNISIYKVRTDVLGNQHIIKNEGDKPKLDF

SEQ ID NO: 314 - protein, 1388 aa, synthetic construct
MTKPYSIGLDIGTNSVGWAVTTDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKAYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKKLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDHIIPQAFLKDNSIDNKVLVSSASNRGKSDDVPSLEVVKKRKTFWYQLLKSKLISQRKFDNLTKAERGGLSPEDKAGFIQRQLVETRQITKHVARLLDEKFNNKKDENNRAVRTVKIITLKSTLVSQFRKDFELYKVREINDFHHAHDAYLNAVVASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFTVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 315 - protein, 1082 aa, synthetic construct
MAAFKPNSINYILGLDIGIASVGWAMVEIDEEENPIRLIDLGVRVFERAEVPKTGDSLAMARRLARSVRRLTRRRAHRLLRTRRLLKREGVLQAANFDENGLIKSLPNTPWQLRAAALDRKLTPLEWSAVLLHLIKHRGYLSQRKNEGETADKELGALLKGVAGNAHALQTGDFRTPAELALNKFEKESGHIRNQRSDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTLEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 316 - protein, 1053 aa, synthetic construct
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 317 - protein, 1307 aa, synthetic construct
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 318 - protein, 1229 aa, synthetic construct
MAASKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGAAGYKSLFKKDIIETILPEAADDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNAKTKQALPKFKPLYKQVLSDRESLSFYGEGYTSDEEVLEVFRNTLNKNSEIFSSIKKLEKLFKNFDEYSSAGIFVKNGPAISTISKDIFGEWNLIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFAAAKKNNVFAWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVK

SEQ ID NO: 319 - protein, 1300 aa, synthetic construct
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 320 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGD

SEQ ID NO: 321 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFESPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGD

SEQ ID NO: 322 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKEYRSTKEVLDATLIHQSITGLYETRIDLSQLGGD

SEQ ID NO: 323 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARFLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPRAFKYFDTTIDRKVYRSTKEVLDATLIHQSITGLYETRIDLSQLGGD

SEQ ID NO: 324 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDTKLQLSKDTYDDDLDNLLAQIGDQYADLFLAAKNLSDAILLSDILRVNTEITKAPLSASMIKLYDEHHQDLTLLKALVRQQLPEKYKEIFFDQSKNGYAGYIDGGASQEEFYKFIKPILEKMDGTEELLVKLNREDLLRKQRTFDNGIIPHQIHLGELHAILRRQEDFYPFLKDNREKIEKILTFRIPYYVGPLARGNSRFAWMTRKSEETITPWNFEKVVDKGASAQSFIERMTNFDKNLPNEKVLPKHSLLYEYFTVYNELTKVKYVTEGMRKPAFLSGDQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFIQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGVLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 325 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLADDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPALESEFVYGDYKVYDVRKMIAKSEQEIGKATAKYFFYSNIMNFFKTEITLANGEIRKAPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 326 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTAFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGALSRKLINGIRDKQSGKTILDFLKSDGFANRNFMALIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRAITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 327 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRAFAALIADDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 328 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANANFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 329 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPASLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEIARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNANLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 330 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGD

SEQ ID NO: 331 - protein, 1368 aa, synthetic construct
MDKKYSIGLDIGTNSVGWAVITDEYKVPSKKFKVLGNTDRHSIKKNLIGALLFDSGETAERTRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTRLGAPRAFKYFDTTIDPKQYRSTKEVLDATLIHQSITGLYETRIDLSQLGGD

SEQ ID NO: 332 - protein, 1054 aa, synthetic construct
MGKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRKLINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYKNDLIKINGELYRVIGVNNDLLNRIEVNMIDITYREYLENMNDKRPPHIIKTIASKTQSIKKYSTDILGNLYEVKSKKHPQIIKKG

SEQ ID NO: 333 - protein, 984 aa, synthetic construct
MARILAFDIGISSIGWAFSENDELKDCGVRIFTKVENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEEKLANYQSVGEYLYKEYFQKFKENSKEFTNVRNKKESYERCIAQSFLKDELKLIFKKQREFGFSFSKKFEEEVLSVAFYKRALKDFSHLVGNCSFFTDEKRAPKNSPLAFMFVALTRIINLLNNLKNTEGILYTKDDLNALLNEVLKNGTLTYKQTKKLLGLSDDYEFKGEKGTYFIEFKKYKEFIKALGEHNLSQDDLNEIAKDITLIKDEIKLKKALAKYDLNQNQIDSLSKLEFKDHLNISFKALKLVTPLMLEGKKYDEACNELNLKVAINEDKKDFLPAFNETYYKDEVTNPVVLRAIKEYRKVLNALLKKYGKVHKINIELAREVGKNHSQRAKIEKEQNENYKAKKDAELECEKLGLKINSKNILKLRLFKEQKEFCAYSGEKIKISDLQDEKMLEIDHIYPYSRSFDDSYMNKVLVFTKQNQEKLNQTPFEAFGNDSAKWQKIEVLAKNLPTKKQKRILDKNYKDKEQKNFKDRNLNDTRYIARLVLNYTKDYLDFLPLSDDENTKLNDTQKGSKVHVEAKSGMLTSALRHTWGFSAKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPEFVYYNAFTSSTVSLIVSKHDNKFETLSKNQKILFKNANEKEVIAKSIGIQNLKVFEKYIVSALGEVTKAEFRQREDFKK

SEQ ID NO: 334 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMVKRYDEHHQDLTLLKALVRQQLPEKYKEIFFDQSKNGYAGYIDGGASQEEFYKFIKPILEKMDGTEELLVKLNREDLLRKQRTFDNGIIPHQIHLGELHAILRRQGDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRLRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGGHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKGNSDKLIARKKDWDPKKYGGFNSPTAAYSVLVVAKVEKGKSKKLKSVKELLGITIMERSSFEKNPIGFLEAKGYKEVKKDLIIKLPKYSLFELENGRKRMLASAGVLHKGNELALPSKYVNFLYLASHYEKLKGSPEDNEQKQLFVEQHKHYLDEIIEQISEFSKRVILADANLDKVLSAYNKHRDKPIREQAENIIHLFTLTNLGVPAAFKYFDTTIDKKRYTSTKEVLDATLIHQSITGLYETRIDLSQLGGD

SEQ ID NO: 335 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMVKRYDEHHQDLTLLKALVRQQLPEKYKEIFFDQSKNGYAGYIDGGASQEEFYKFIKPILEKMDGTEELLVKLNREDLLRKQRTFDNGIIPHQIHLGELHAILRRQGDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRLRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGGHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKGNSDKLIARKKDWDPKKYGGFNSPTVAYSVLVVAKVEKGKSKKLKSVKELLGITIMERSSFEKNPIDFLEAKGYKEVKKDLIIKLPKYSLFELENGRKRMLASAGVLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTINRKQYNTTKEVLDATLIRQSITGLYETRIDLSQLGGD

SEQ ID NO: 336 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMVKRYDEHHQDLTLLKALVRQQLPEKYKEIFFDQSKNGYAGYIDGGASQEEFYKFIKPILEKMDGTEELLVKLNREDLLRKQRTFDNGIIPHQIHLGELHAILRRQGDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRLRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGGHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKGNSDKLIARKKDWDPKKYGGFNSPTVAYSVLVVAKVEKGKSKKLKSVKELLGITIMERSSFEKNPIGFLEAKGYKEVKKDLIIKLPKYSLFELENGRKRMLASASVLHKGNELALPSKYVNFLYLASHYEKLKGSSEDNKQKQLFVEQHKHYLDEIIEQISEFSKRVILADANLDKVLSAYNKHRDKPIREQAENIIHLFTLTNLGASAAFKYFDTTIGRKLYTSTKEVLDATLIHQSITGLYETRIDLSQLGGD

SEQ ID NO: 337 - protein, 1250 aa, synthetic construct
MQTLFENFTNQYPVSKTLRFELIPQGKTKDFIEQKGLLKKDEDRAEKYKKVKNIIDEYHKDFIEKSLNGLKLDGLEKYKTLYLKQEKDDKDKKAFDKEKENLRKQIANAFRNNEKFKTLFAKELIKNDLMSFACEEDKKNVKEFEAFTTYFTGFHQNRANMYVADEKRTAIASRLIHENLPKFIDNIKIFEKMKKEAPELLSPFNQTLKDMKDVIKGTTLEEIFSLDYFNKTLTQSGIDIYNSVIGGRTPEEGKTKIKGLNEYINTDFNQKQTDKKKRQPKFKQLYKQILSDRQSLSFIAEAFKNDTEILEAIEKFYVNELLHFSNEGKSTNVLDAIKNAVSNLESFNLTKMYFRSGASLTDVSRKVFGEWSIINRALDNYYATTYPIKPREKSEKYEERKEKWLKQDFNVSLIQTAIDEYDNETVKGKNSGKVIADYFAKFCDDKETDLIQKVNEGYIAVKDLLNTPCPENEKLGSNKDQVKQIKAFMDSIMDIMHFVRPLSLKDTDKEKDETFYSLFTPLYDHLTQTIALYNKVRNYLTQKPYSTEKIKLNFENSTLLGGWDLNKETDNTAIILRKDNLYYLGIMDKRHNRIFRNVPKADKKDFCYEKMVYKLLPGANKMLPKVFFSQSRIQEFTPSAKLLENYANETHKKGDNFNLNHCHKLIDFFKDSINKHEDWKNFDFRFSATSTYADLSGFYHEVEHQGYKISFQSVADSFIDDLVNEGKLYLFQIYNKDFSPFSKGKPNLHTLYWKMLFDENNLKDVVYKLNGEAEVFYRKKSIAEKNTTIHKANESIINKNPDNPKATSTFNYDIVKDKRYTIDKFQFHIPITMNFKAEGIFNMNQRVNQFLKANPDINIIGIDRGERHLLYYALINQKGKILKQDTLNVIANEKQKVDYHNLLDKKEGDRATARQEWGVIETIKELKEGYLSQVIHKLTDLMIENNAIIVMEDLNFGFKRGRQKVEKQVYQKFEKMLIDKLNYLVDKNKKANELGGLLNAFQLANKFESFQKMGKQNGFIFYVPAWNTSKTDPATGFIDFLKPRYENLNQAKDFFEKFDSIRLNSKADYFEFAFDFKNFTEKADGGRTKWTVCTTNEDRYAWNRALNNNRGSQEKYDITAELKSLFDGKVDYKSGKDLKQQIASQESADFFKALMKNLSITLSLRHNNGEKGDNEQDYILSPVADSKGRFFDSRKADDDMPKNADANGAYHIALKGLWCLEQISKTDDLKKVKLAISNKEWLEFVQTLKG

SEQ ID NO: 338 - protein, 1260 aa, synthetic construct
MDSLKDFTNLYPVSKTLRFELKPVGKTLENIEKAGILKEDEHRAESYRRVKKIIDTYHKVFIDSSLENMAKMGIENEIKAMLQSFCELYKKDHRTEGEDKALDKIRAVLRGLIVGAFTGVCGRRENTVQNEKYESLFKEKLIKEILPDFVLSTEAESLPFSVEEATRSLKEFDSFTSYFAGFYENRKNIYSTKPQSTAIAYRLIHENLPKFIDNILVFQKIKEPIAKELEHIRADFSAGGYIKKDERLEDIFSLNYYIHVLSQAGIEKYNALIGKIVTEGDGEMKGLNEHINLYNQQRGREDRLPLFRPLYKQILSDREQLSYLPESFEKDEELLRALKEFYDHIAEDILGRTQQLMTSISEYDLSRIYVRNDSQLTDISKKMLGDWNAIYMARERAYDHEQAPKRITAKYERDRIKALKGEESISLANLNSCIAFLDNVRDCRVDTYLSTLGQKEGPHGLSNLVENVFASYHEAEQLLSFPYPEENNLIQDKDNVVLIKNLLDNISDLQRFLKPLWGMGDEPDKDERFYGEYNYIRGALDQVIPLYNKVRNYLTRKPYSTRKVKLNFGNSQLLSGWDRNKEKDNSCVILRKGQNFYLAIMNNRHKRSFENKMLPEYKEGEPYFEKMDYKFLPDPNKMLPKVFLSKKGIEIYKPSPKLLEQYGHGTHKKGDTFSMDDLHELIDFFKHSIEAHEDWKQFGFKFSDTATYENVSSFYREVEDQGYKLSFRKVSESYVYSLIDQGKLYLFQIYNKDFSPCSKGTPNLHTLYWRMLFDERNLADVIYKLDGKAEIFFREKSLKNDHPTHPAGKPIKKKSRQKKGEESLFEYDLVKDRRYTMDKFQFHVPITMNFKCSAGSKVNDMVNAHIREAKDMHVIGIDRGERNLLYICVIDSRGTILDQISLNTINDIDYHDLLESRDKDRQQEHRNWQTIEGIKELKQGYLSQAVHRIAELMVAYKAVVALEDLNMGFKRGRQKVESSVYQQFEKQLIDKLNYLVDKKKRPEDIGGLLRAYQFTAPFKSFKEMGKQNGFLFYIPAWNTSNIDPTTGFVNLFHVQYENVDKAKSFFQKFDSISYNPKKDWFEFAFDYKNFTKKAEGSRSMWILCTHGSRIKNFRNSQKNGQWDSEEFALTEAFKSLFVRYEIDYTADLKTAIVDEKQKDFFVDLLKLFKLTVQMRNSWKEKDLDYLISPVAGADGRFFDTREGNKSLPKDADANGAYNIALKGLWALRQIRQTSEGGKLKLAISNKEWLQFVQERSYEKD

SEQ ID NO: 339 - protein, 1241 aa, synthetic construct
MLLYENYTKRNQITKSLRLELRPQGKTLRNIKELNLLEQDKAIYALLERLKPVIDEGIKDIARDTLKNCELSFEKLYEHFLSGDKKAYAKESERLKKEIVKTLIKNLPEGIGKISEINSAKYLNGVLYDFIDKTHKDSEEKQNILSDILETKGYLALFSKFLTSRITTLEQSMPKRVIENFEIYAANIPKMQDALERGAVSFAIEYESICSVDYYNQILSQEDIDSYNRLISGIMDEDGAKEKGINQTISEKNIKIKSEHLEEKPFRILKQLHKQILEEREKAFTIDHIDSDEEVVQVTKEAFEQTKEQWENIKKINGFYAKDPGDITLFIVVGPNQTHVLSQLIYGEHDRIRLLLEEYEKNTLEVLPRRTKSEKARYDKFVNAVPKKVAKESHTFDGLQKMTGDDRLFILYRDELARNYMRIKEAYGTFERDILKSRRGIKGNRDVQESLVSFYDELTKFRSALRIINSGNDEKADPIFYNTFDGIFEKANRTYKAENLCRNYVTKSPADDARIMASCLGTPARLRTHWWNGEENFAINDVAMIRRGDEYYYFVLTPDVKPVDLKTKDETDAQIFVQRKGAKSFLGLPKALFKCILEPYFESPEHKNDKNCVIEEYVSKPLTIDRRAYDIFKNGTFKKTNIGIDGLTEEKFKDDCRYLIDVYKEFIAVYTRYSCFNMSGLKRADEYNDIGEFFSDVDTRLCTMEWIPVSFERINDMVDKKEGLLFLVRSMFLYNRPRKPYERTFIQLFSDSNMEHTSMLLNSRAMIQYRAASLPRRVTHKKGSILVALRDSNGEHIPMHIREAIYKMKNNFDISSEDFIMAKAYLAEHDVAIKKANEDIIRNRRYTEDKFFLSLSYTKNADISARTLDYINDKVEEDTQDSRMAVIVTRNLKDLTYVAVVDEKNNVLEEKSLNEIDGVNYRELLKERTKIKYHDKTRLWQYDVSSKGLKEAYVELAVTQISKLATKYNAVVVVESMSSTFKDKFSFLDEQIFKAFEARLCARMSDLSFNTIKEGEAGSISNPIQVSNNNGNSYQDGVIYFLNNAYTRTLCPDTGFVDVFDKTRLITMQSKRQFFAKMKDIRIDDGEMLFTFNLEEYPTKRLLDRKEWTVKIAGDGSYFDKDKGEYVYVNDIVREQIIPALLEDKAVFDGNMAEKFLDKTAISGKSVELIYKWFANALYGIITKKDGEKIYRSPITGTEIDVSKNTTYNFGKKFMFKQEYRGDGDFLDAFLNYMQAQDIAV

SEQ ID NO: 340 - protein, 1263 aa, synthetic construct
MEDYSGFVNIYSIQKTLRFELKPVGKTLEHIEKKGFLKKDKIRAEDYKAVKKIIDKYHRAYIEEVFDSVLHQKKKKDKTRFSTQFIKEIKEFSELYYKTEKNIPDKERLEALSEKLRKMLVGAFKGEFSEEVAEKYKNLFSKELIRNEIEKFCETDEERKQVSNFKSFTTYFTGFHSNRQNIYSDEKKSTAIGYRIIHQNLPKFLDNLKIIESIQRRFKDFPWSDLKKNLKKIDKNIKLTEYFSIDGFVNVLNQKGIDAYNTILGGKSEESGEKIQGLNEYINLYRQKNNIDRKNLPNVKILFKQILGDRETKSFIPEAFPDDQSVLNSITEFAKYLKLDKKKKSIIAELKKFLSSFNRYELDGIYLANDNSLASISTFLFDDWSFIKKSVSFKYDESVGDPKKKIKSPLKYEKEKEKWLKQKYYTISFLNDAIESYSKSQDEKRVKIRLEAYFAEFKSKDDAKKQFDLLERIEEAYAIVEPLLGAEYPRDRNLKADKKEVGKIKDFLDSIKSLQFFLKPLLSAEIFDEKDLGFYNQLEGYYEEIDSIGHLYNKVRNYLTGKIYSKEKFKLNFENSTLLKGWDENREVANLCVIFREDQKYYLGVMDKENNTILSDIPKVKPNELFYEKMVYKLIPTPHMQLPRIIFSSDNLSIYNPSKSILKIREAKSFKEGKNFKLKDCHKFIDFYKESISKNEDWSRFDFKFSKTSSYENISEFYREVERQGYNLDFKKVSKFYIDSLVEDGKLYLFQIYNKDFSIFSKGKPNLHTIYFRSLFSKENLKDVCLKLNGEAEMFFRKKSINYDEKKKREGHHPELFEKLKYPILKDKRYSEDKFQFHLPISLNFKSKERLNFNLKVNEFLKRNKDINIIGIDRGERNLLYLVMINQKGEILKQTLLDSMQSGKGRPEINYKEKLQEKEIERDKARKSWGTVENIKELKEGYLSIVIHQISKLMVENNAIVVLEDLNIGFKRGRQKVERQVYQKFEKMLIDKLNFLVFKENKPTEPGGVLKAYQLTDEFQSFEKLSKQTGFLFYVPSWNTSKIDPRTGFIDFLHPAYENIEKAKQWINKFDSIRFNSKMDWFEFTADTRKFSENLMLGKNRVWVICTTNVERYFTSKTANSSIQYNSIQITEKLKELFVDIPFSNGQDLKPEILRKNDAVFFKSLLFYIKTTLSLRQNNGKKGEEEKDFILSPVVDSKGRFFNSLEASDDEPKDADANGAYHIALKGLMNLLVLNETKEENLSRPKWKIKNKDWLEFVWERNR

SEQ ID NO: 341 - protein, 1246 aa, synthetic construct
MKTQHFFEDFTSLYSLSKTIRFELKPIGKTLENIKKNGLIRRDEQRLDDYEKLKKVIDEYHEDFIANILSSFSFSEEILQSYIQNLSESEARAKIEKTMRDTLAKAFSEDERYKSIFKKELVKKDIPVWCPAYKSLCKKFDNFTTSLVPFHENRKNLYTSNEITASIPYRIVHVNLPKFIQNIEALCELQKKMGADLYLEMMENLRNVWPSFVKTPDDLCNLKTYNHLMVQSSISEYNRFVGGYSTEDGTKHQGINEWINIYRQRNKEMRLPGLVFLHKQILAKVDSSSFISDTLENDDQVFCVLRQFRKLFWNTVSSKEDDAASLKDLFCGLSGYDPEAIYVSDAHLATISKNIFDRWNYISDAIRRKTEVLMPRKKESVERYAEKISKQIKKRQSYSLAELDDLLAHYSEESLPAGFSLLSYFTSLGGQKYLVSDGEVILYEEGSNIWDEVLIAFRDLQVILDKDFTEKKLGKDEEAVSVIKKALDSALRLRKFFDLLSGTGAEIRRDSSFYALYTDRMDKLKGLLKMYDKVRNYLTKKPYSIEKFKLHFDNPSLLSGWDKNKELNNLSVIFRQNGYYYLGIMTPKGKNLFKTLPKLGAEEMFYEKMEYKQIAEPMLMLPKVFFPKKTKPAFAPDQSVVDIYNKKTFKTGQKGFNKKDLYRLIDFYKEALTVHEWKLFNFSFSPTEQYRNIGEFFDEVREQAYKVSMVNVPASYIDEAVENGKLYLFQIYNKDFSPYSKGIPNLHTLYWKALFSEQNQSRVYKLCGGGELFYRKASLHMQDTTVHPKGISIHKKNLNKKGETSLFNYDLVKDKRFTEDKFFFHVPISINYKNKKITNVNQMVRDYIAQNDDLQIIGIDRGERNLLYISRIDTRGNLLEQFSLNVIESDKGDLRTDYQKILGDREQERLRRRQEWKSIESIKDLKDGYMSQVVHKICNMVVEHKAIVVLENLNLSFMKGRKKVEKSVYEKFERMLVDKLNYLVVDKKNLSNEPGGLYAAYQLTNPLFSFEELHRYPQSGILFFVDPWNTSLTDPSTGFVNLLGRINYTNVGDARKFFDRFNAIRYDGKGNILFDLDLSRFDVRVETQRKLWTLTTFGSRIAKSKKSGKWMVERIENLSLCFLELFEQFNIGYRVEKDLKKAILSQDRKEFYVRLIYLFNLMMQIRNSDGEEDYILSPALNEKNLQFDSRLIEAKDLPVDADANGAYNVARKGLMVVQRIKRGDHESIHRIGRAQWLRYVQEGIVE

SEQ ID NO: 342 - protein, 1206 aa, synthetic construct
MYYESLTKQYPVSKTIRNELIPIGKTLDNIRQNNILESDVKRKQNYEHVKGILDEYHKQLINEALDNCTLPSLKIAAEIYLKNQKEVSDREDFNKTQDLLRKEVVEKLKAHENFTKIGKKDILDLLEKLPSISEDDYNALESFRNFYTYFTSYNKVRENLYSDKEKSSTVAYRLINENFPKFLDNVKSYRFVKTAGILADGLGEEEQDSLFIVETFNKTLTQDGIDTYNSQVGKINSSINLYNQKNQKANGFRKIPKMKMLYKQILSDREESFIDEFQSDEVLIDNVESYGSVLIESLKSSKVSAFFDALRESKGKNVYVKNDLAKTAMSNIVFENWRTFDDLLNQEYDLANENKKKDDKYFEKRQKELKKNKSYSLEHLCNLSEDSCNLIENYIHQISDDIENIIINNETFLRIVINEHDRSRKLAKNRKAVKAIKDFLDSIKVLERELKLINSSGQELEKDLIVYSAHEELLVELKQVDSLYNMTRNYLTKKPFSTEKVKLNFNRSTLLNGWDRNKETDNLGVLLLKDGKYYLGIMNTSANKAFVNPPVAKTEKVFKKVDYKLLPVPNQMLPKVFFAKSNIDFYNPSSEIYSNYKKGTHKKGNMFSLEDCHNLIDFFKESISKHEDWSKFGFKFSDTASYNDISEFYREVEKQGYKLTYTDIDETYINDLIERNELYLFQIYNKDFSMYSKGKLNLHTLYFMMLFDQRNIDDVVYKLNGEAEVFYRPASISEDELIIHKAGEEIKNKNPNRARTKETSTFSYDIVKDKRYSKDKFTLHIPITMNFGVDEVKRFNDAVNSAIRIDENVNVIGIDRGERNLLYVVVIDSKGNILEQISLNSIINKEYDIETDYHALLDEREGGRDKARKDWNTVENIRDLKAGYLSQVVNVVAKLVLKYNAIICLEDLNFGFKRGRQKVEKQVYQKFEKMLIDKLNYLVIDKSREQTSPKELGGALNALQLTSKFKSFKELGKQSGVIYYVPAYLTSKIDPTTGFANLFYMKCENVEKSKRFFDGFDFIRFNALENVFEFGFDYRSFTQRACGINSKWTVCTNGERIIKYRNPDKNNMFDEKVVVVTDEMKNLFEQYKIPYEDGRNVKDMIISNEEAEFYRRLYRLLQQTLQMRNSTSDGTRDYIISPVKNKREAYFNSELSDGSVPKDADANGAYNIARKGLWVLEQIRQKSEGEKINLAMTNAEWLEYAQTHLL

SEQ ID NO: 343 - protein, 1352 aa, synthetic construct
MENIFDQFIGKYSLSKTLRFELKPVGKTEDFLKINKVFEKDQTIDDSYNQAKFYFDSLHQKFIDAALASDKTSELSFQNFADVLEKQNKIILDKKREMGALRKRDKNAVGIDRLQKEINDAEDIIQKEKEKIYKDVRTLFDNEAESWKTYYQEREVDGKKITFSKADLKQKGADFLTAAGILKVLKYEFPEEKEKEFQAKNQPSLFVEEKENPGQKRYIFDSFDKFAGYLTKFQQTKKNLYAADGTSTAVATRIADNFIIFHQNTKVFRDKYKNNHTDLGFDEENIFEIERYKNCLLQREIEHIKNENSYNKIIGRINKKIKEYRDQKAKDTKLTKSDFPFFKNLDKQILGEVEKEKQLIEKTREKTEEDVLIERFKEFIENNEERFTAAKKLMNAFCNGEFESEYEGIYLKNKAINTISRRWFVSDRDFELKLPQQKSKNKSEKNEPKVKKFISIAEIKNAVEELDGDIFKAVFYDKKIIAQGGSKLEQFLVIWKYEFEYLFRDIERENGEKLLGYDSCLKIAKQLGIFPQEKEAREKATAVIKNYADAGLGIFQMMKYFSLDDKDRKNTPGQLSTNFYAEYDGYYKDFEFIKYYNEFRNFITKKPFDEDKIKLNFENGALLKGWDENKEYDFMGVILKKEGRLYLGIMHKNHRKLFQSMGNAKGDNANRYQKMIYKQIADASKDVPRLLLTSKKAMEKFKPSQEILRIKKEKTFKRESKNFSLRDLHALIEYYRNCIPQYSNWSFYDFQFQDTGKYQNIKEFTDDVQKYGYKISFRDIDDEYINQALNEGKMYLFEVVNKDIYNTKNGSKNLHTLYFEHILSAENLNDPVFKLSGMAEIFQRQPSVNEREKITTQKNQCILDKGDRAYKYRRYTEKKIMFHMSLVLNTGKGEIKQVQFNKIINQRISSSDNEMRVNVIGIDRGEKNLLYYSVVKQNGEIIEQASLNEINGVNYRDKLIEREKERLKNRQSWKPVVKIKDLKKGYISHVIHKICQLIEKYSAIVVLEDLNMRFKQIRGGIERSVYQQFEKALIDKLGYLVFKDNRDLRAPGGVLNGYQLSAPFVSFEKMRKQTGILFYTQAEYTSKTDPITGFRKNVYISNSASLDKIKEAVKKFDAIGWDGKEQSYFFKYNPYNLADEKYKNSTVSKEWAIFASAPRIRRQKGEDGYWKYDRVKVNEEFEKLLKVWNFVNPKATDIKQEIIKKEKAGDLQGEKELDGRLRNFWHSFIYLFNLVLELRNSFSLQIKIKAGEVIAVDEGVDFIASPVKPFFTTPNPYIPSNLCWLAVENADANGAYNIARKGVMILKKIREHAKKDPEFKKLPNLFISNAEWDEAARDWGKYAGTTALNLDH

SEQ ID NO: 344 - protein, 1477 aa, synthetic construct
MSNFFKNFTNLYELSKTLRFELKPVGDTLTNMKDHLEYDEKLQTFLKDQNIDDAYQALKPQFDEIHEEFITDSLESKKAKEIDFSEYLDLFQEKKELNDSEKKLRNKIGETFNKAGEKWKKEKYPQYEWKKGSKIANGADILSCQDMLQFIKYKNPEDEKIKNYIDDTLKGFFTYFGGFNQNRANYYETKKEASTAVATRIVHENLPKFCDNVIQFKHIIKRKKDGTVEKTERKTEYLNAYQYLKNNNKITQIKDAETEKMIESTPIAEKIFDVYYFSSCLSQKQIEEYNRIIGHYNLLINLYNQAKRSEGKHLSANEKKYKDLPKFKTLYKQIGCGKKKDLFYTIKCDTEEEANKSRNEGKESHSVEEIINKAQEAINKYFKSNNDCENINTVPDFINYILTKENYEGVYWSKAAMNTISDKYFANYHDLQDRLKEAKVFQKADKKSEDDIKIPEAIELSGLFGVLDSLADWQTTLFKSSILSNEDKLKIITDSQTPSEALLKMIFNDIEKNMESFLKETNDIITLKKYKGNKEGTEKIKQWFDYTLAINRMLKYFLVKENKIKGNSLDTNISEALKTLIYSDDAEWFKWYDALRNYLTQKPQDEAKENKLKLNFDNPSLAGGWDVNKECSNFCVILKDKNEKKYLAIMKKGENTLFQKEWTEGRGKNLTKKSNPLFEINNCEILSKMEYDFWADVSKMIPKCSTQLKAVVNHFKQSDNEFIFPIGYKVTSGEKFREECKISKQDFELNNKVFNKNELSVTAMRYDLSSTQEKQYIKAFQKEYWELLFKQEKRDTKLTNNEIFNEWINFCNKKYSELLSWERKYKDALTNWINFCKYFLSKYPKTTLFNYSFKESENYNSLDEFYRDVDICSYKLNINTTINKSILDRLVEEGKLYLFEIKNQDSNDGKSIGHKNNLHTIYWNAIFENFDNRPKLNGEAEIFYRKAISKDKLGIVKGKKTKNGTEIIKNYRFSKEKFILHVPITLNFCSNNEYVNDIVNTKFYNFSNLHFLGIDRGEKHLAYYSLVNKNGEIVDQGTLNLPFTDKDGNQRSIKKEKYFYNKQEDKWEAKEVDCWNYNDLLDAMASNRDMARKNWQRIGTIKEAKNGYVSLVIRKIADLAVNNERPAFIVLEDLNTGFKRSRQKIDKSVYQKFELALAKKLNFLVDKNAKRDEIGSPTKALQLTPPVNNYGDIENKKQAGIMLYTRANYTSQTDPATGWRKTIYLKAGPEETTYKKDGKIKNKSVKDQIIETFTDIGFDGKDYYFEYDKGEFVDEKTGEIKPKKWRLYSGENGKSLDRFRGEREKDKYEWKIDKIDIVKILDDLFVNFDKNISLLKQLKEGVELTRNNEHGTGESLRFAINLIQQIRNTGNNERDNDFILSPVRDENGKHFDSREYWDKETKGEKISMPSSGDANGAFNIARKGIIMNAHILANSDSKDLSLFVSDEEWDLHLNNKTEWKKQLNIFSSRKAMAKRKK

SEQ ID NO: 345 - protein, 1323 aa, synthetic construct
MENYQEFTNLFQLNKTLRFELKPIGKTCELLEEGKIFASGSFLEKDKVRADNVSYVKKEIDKKHKIFIEETLSSFSISNDLLKQYFDCYNELKAFKKDCKSDEEEVKKTALRNKCTSIQRAMREAISQAFLKSPQKKLLAIKNLIENVFKADENVQHFSEFTSYFSGFETNRENFYSDEEKSTSIAYRLVHDNLPIFIKNIYIFEKLKEQFDAKTLSEIFENYKLYVAGSSLDEVFSLEYFNNTLTQKGIDNYNAVIGKIVKEDKQEIQGLNEHINLYNQKHKDRRLPFFISLKKQILSDREALSWLPDMFKNDSEVIKALKGFYIEDGFENNVLTPLATLLSSLDKYNLNGIFIRNNEALSSLSQNVYRNFSIDEAIDANAELQTFNNYELIANALRAKIKKETKQGRKSFEKYEEYIDKKVKAIDSLSIQEINELVENYVSEFNSNSGNMPRKVEDYFSLMRKGDFGSNDLIENIKTKLSAAEKLLGTKYQETAKDIFKKDENSKLIKELLDATKQFQHFIKPLLGTGEEADRDLVFYGDFLPLYEKFEELTLLYNKVRNRLTQKPYSKDKIRLCFNKPKLMTGWVDSKTEKSDNGTQYGGYLFRKKNEIGEYDYFLGISSKAQLFRKNEAVIGDYERLDYYQPKANTIYGSAYEGENSYKEDKKRLNKVIIAYIEQIKQTNIKKSIIESISKYPNISDDDKVTPSSLLEKIKKVSIDSYNGILSFKSFQSVNKEVIDNLLKTISPLKNKAEFLDLINKDYQIFTEVQAVIDEICKQKTFIYFPISNVELEKEMGDKDKPLCLFQISNKDLSFAKTFSANLRKKRGAENLHTMLFKALMEGNQDNLDLGSGAIFYRAKSLDGNKPTHPANEAIKCRNVANKDKVSLFTYDIYKNRRYMENKFLFHLSIVQNYKAANDSAQLNSSATEYIRKADDLHIIGIDRGERNLLYYSVIDMKGNIVEQDSLNIIRNNDLETDYHDLLDKREKERKANRQNWEAVEGIKDLKKGYLSQAVHQIAQLMLKYNAIIALEDLGQMFVTRGQKIEKAVYQQFEKSLVDKLSYLVDKKRPYNELGGILKAYQLASSITKNNSDKQNGFLFYVPAWNTSKIDPVTGFTDLLRPKAMTIKEAQDFFGAFDNISYNDKGYFEFETNYDKFKIRMKSAQTRWTICTFGNRIKRKKDKNYWNYEEVELTEEFKKLFKDSNIDYENCNLKEEIQNKDNRKFFDDLIKLLQLTLQMRNSDDKGNDYIISPVANAEGQFFDSRNGDKKLPLDADANGAYNIARKGLWNIRQIKQTKNDKKLNLSISSTEWLDFVREKPYLK

SEQ ID NO: 346 - protein, 1373 aa, synthetic construct
MLFQDFTHLYPLSKTVRFELKPIDRTLEHIHAKNFLSQDETMADMHQKVKVILDDYHRDFIADMMGEVKLTKLAEFYDVYLKFRKNPKDDELQKQLKDLQAVLRKEIVKPIGNGGKYKAGYDRLFGAKLFKDGKELGDLAKFVIAQEGESSPKLAHLAHFEKFSTYFTGFHDNRKNMYSDEDKHTAIAYRLIHENLPRFIDNLQILTTIKQKHSALYDQIINELTASGLDVSLASHLDGYHKLLTQEGITAYNTLLGGISGEAGSPKIQGINELINSHHNQHCHKSERIAKLRPLHKQILSDGMSVSFLPSKFADDSEMCQAVNEFYRHYADVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIEHYTARHDDESVQAGKLGQYFKHGLAGVDNPIQKIHNNHSTIKGFLERERPAGERALPKIKSGKNPEMTQLRQLKELLDNALNVAHFAKLLTTKTTLDNQDGNFYGEFGVLYDELAKIPTLYNKVRDYLSQKPFSTEKYKLNFGNPTLLNGWDLNKEKDNFGVILQKDGCYYLALLDKAHKKVFDNAPNTGKSIYQKMIYKYLEVRKQFPKVFFSKEAIAINYHPSKELVEIKDKGRQRSDDERLKLYRFILECLKIHPKYDKKFEGAIGDIQLFKKDKKGREVPISEKDLFDKINGIFSSKPKLEMEDFFIGEFKRYNPSQDLVDQYNIYKKIDSNDNRKKENFYNNHPKFKKDLVRYYYESMCKHEEWEESFEFSKKLQDIGCYVDVNELFTEIETRRLNYKISFCNINADYIDELVEQGQLYLFQIYNKDFSPKAHGKPNLHTLYFKALFSEDNLADPIYKLNGEAQIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQCSLNDITTASANGTQMTTPYHKILDKREIERLNARVGWGEIETIKELKSGYLSHVVHQISQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIAQSQAFFGKFDKICYNADKDYFEFHIDYAKFTDKAKNSRQIWTICSHGDKRYVYDKTANQNKGAAKGINVNDELKSLFARHHINEKQPNLVMDICQNNDKEFHKSLMYLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 347 - protein, 1282 aa, synthetic construct
MNGNRSIVYREFVGVIPVAKTLRNELRPVGHTQEHIIQNGLIQEDELRQEKSTELKNIMDDYYREYIDKSLSGVTDLDFTLLFELMNLVQSSPSKDNKKALEKEQSKMREQICTHLQSDSNYKNIFNAKLLKEILPDFIKNYNQYDVKDKAGKLETLALFNGFSTYFTDFFEKRKNVFTKEAVSTSIAYRIVHENSLIFLANMTSYKKISEKALDEIEVIEKNNQDKMGDWELNQIFNPDFYNMVLIQSGIDFYNEICGVVNAHMNLYCQQTKNNYNLFKMRKLHKQILAYTSTSFEVPKMFEDDMSVYNAVNAFIDETEKGNIIGKLKDIVNKYDELDEKRIYISKDFYETLSCFMSGNWNLITGCVENFYDENIHAKGKSKEEKVKKAVKEDKYKSINDVNDLVEKYIDEKERNEFKNSNAKQYIREISNIITDTETAHLEYDDHISLIESEEKADEMKKRLDMYMNMYHWAKAFIVDEVLDRDEMFYSDIDDIYNILENIVPLYNRVRNYVTQKPYNSKKIKLNFQSPTLANGWSQSKEFDNNAIILIRDNKYYLAIFNAKNKPDKKIIQGNSDKKNDNDYKKMVYNLLPGANKMLPKVFLSKKGIETFKPSDYIISGYNAHKHIKTSENFDISFCRDLIDYFKNSIEKHAEWRKYEFKFSATDSYSDISEFYREVEMQGYRIDWTYISEADINKLDEEGKIYLFQIYNKDFAENSTGKENLHTMYFKNIFSEENLKDIIIKLNGQAELFYRRASVKNPVKHKKDSVLVNKTYKNQLDNGDVVRIPIPDDIYNEIYKMYNGYIKESDLSEAAKEYLDKVEVRTAQKDIVKDYRYTVDKYFIHTPITINYKVTARNNVNDMVVKYIAQNDDIHVIGIDRGERNLIYISVIDSHGNIVKQKSYNILNNYDYKKKLVEKEKTREYARKNWKSIGNIKELKEGYISGVVHEIAMLIVEYNAIIAMEDLNYGFKRGRFKVERQVYQKFESMLINKLNYFASKEKSVDEPGGLLKGYQLTYVPDNIKNLGKQCGVIFYVPAAFTSKIDPSTGFISAFNFKSISTNASRKQFFMQFDEIRYCAEKDMFSFGFDYNNFDTYNITMGKTQWTVYTNGERLQSEFNNARRTGKTKSINLTETIKLLLEDNEINYADGHDIRIDMEKMDEDKKSEFFAQLLSLYKLTVQMRNSYTEAEEQENGISYDKIISPVINDEGEFFDSDNYKESDDKECKMPKDADANGAYCIALKGLYEVLKIKSEWTEDGFDRNCLKLPHAEWLDFIQNKRYE

SEQ ID NO: 348 - protein, 1238 aa, synthetic construct
MNNYDEFTKLYPIQKTIRFELKPQGRTMEHLETFNFFEEDRDRAEKYKILKEAIDEYHKKFIDEHLTNMSLDWNSLKQISEKYYKSREEKDKKVFLSEQKRMRQEIVSEFKKDDRFKDLFSKKLFSELLKEEIYKKGNHQEIDALKSFDKFSGYFIGLHENRKNMYSDGDEITAISNRIVNENFPKFLDNLQKYQEARKKYPEWIIKAESALVAHNIKMDEVFSLEYFNKVLNQEGIQRYNLALGGYVTKSGEKMMGLNDALNLAHQSEKSSKGRIHMTPLFKQILSEKESFSYIPDVFTEDSQLLPSIGGFFAQIENDKDGNIFDRALELISSYAEYDTERIYIRQADINRVSNVIFGEWGTLGGLMREYKADSINDINLERTCKKVDKWLDSKEFALSDVLEAIKRTGNNDAFNEYISKMRTAREKIDAARKEMKFISEKISGDEESIHIIKTLLDSVQQFLHFFNLFKARQDIPLDGAFYAEFDEVHSKLFAIVPLYNKVRNYLTKNNLNTKKIKLNFKNPTLANGWDQNKVYDYASLIFLRDGNYYLGIINPKRKKNIKFEQGSGNGPFYRKMVYKQIPGPNKNLPRVFLTSTKGKKEYKPSKEIIEGYEADKHIRGDKFDLDFCHKLIDFFKESIEKHKDWSKFNFYFSPTESYGDISEFYLDVEKQGYRMHFENISAETIDEYVEKGDLFLFQIYNKDFVKAATGKKDMHTIYWNAAFSPENLQDVVVKLNGEAELFYRDKSDIKEIVHREGEILVNRTYNGRTPVPDKIHKKLTDYHNGRTKDLGEAKEYLDKVRYFKAHYDITKDRRYLNDKIYFHVPLTLNFKANGKKNLNKMVIEKFLSDEKAHIIGIDRGERNLLYYSIIDRSGKIIDQQSLNVIDGFDYREKLNQREIEMKDARQSWNAIGKIKDLKEGYLSKAVHEITKMAIQYNAIVVMEELNYGFKRGRFKVEKQIYQKFENMLIDKMNYLVFKDAPDESPGGVLNAYQLTNPLESFAKLGKQTGILFYVPAAYTSKIDPTTGFVNLFNTSSKTNAQERKEFLQKFESISYSAKDGGIFAFAFDYRKFGTSKTDHKNVWTAYTNGERMRYIKEKKRNELFDPSKEIKEALTSSGIKYDGGQNILPDILRSNNNGLIYTMYSSFIAAIQMRVYDGKEDYIISPIKNSKGEFFRTDPKRRELPIDADANGAYNIALRGELTMRAIAEKFDPDSEKMAKLELKHKDWFEFMQTRGD

SEQ ID NO: 349 - protein, 1206 aa, synthetic construct
MYYQNLTKKYPVSKTIRNELIPIGKTLENIRKNNILESDVKRKQDYEHVKGIMDEYHKQLINEALDNYMLPSLNQAAEIYLKKHVDVEDREEFKKTQDLLRREVTGRLKEHENYTKIGKKDILDLLEKLPSISEEDYNALESFRNFYTYFTSYNKVRENLYSDEEKSSTVAYRLINENLPKFLDNIKSYAFVKAAGVLADCIEEEEQDALFMVETFNMTLTQEGIDMYNYQIGKVNSAINLYNQKNHKVEEFKKIPKMKVLYKQILSDREEVFIGEFKDDETLLSSIGAYGNVLMTYLKSEKINIFFDALRESEGKNVYVKNDLSKTTMSNIVFGSWSAFDELLNQEYDLANENKKKDDKYFEKRQKELKKNKSYTLEQMSNLSKEDISPIENYIERISEDIEKICIYNGEFEKIVVNEHDSSRKLSKNIKAVKVIKDYLDSIKELEHDIKLINGSGQELEKNLVVYVGQEEALEQLRPVDSLYNLTRNYLTKKPFSTEKVKLNFNKSTLLNGWDKNKETDNLGILFFKDGKYYLGIMNTTANKAFVNPPAAKTENVFKKVDYKLLPGSNKMLPKVFFAKSNIGYYNPSTELYSNYKKGTHKKGPSFSIDDCHNLIDFFKESIKKHEDWSKFGFEFSDTADYRDISEFYREVEKQGYKLTFTDIDESYINDLIEKNELYLFQIYNKDFSEYSKGKLNLHTLYFMMLFDQRNLDNVVYKLNGEAEVFYRPASIAENELVIHKAGEGIKNKNPNRAKVKETSTFSYDIVKDKRYSKYKFTLHIPITMNFGVDEVRRFNDVINNALRTDDNVNVIGIDRGERNLLYVVVINSEGKILEQISLNSIINKEYDIETNYHALLDEREDDRNKARKDWNTIENIKELKTGYLSQVVNVVAKLVLKYNAIICLEDLNFGFKRGRQKVEKQVYQKFEKMLIEKLNYLVIDKSREQVSPEKMGGALNALQLTSKFKSFAELGKQSGIIYYVPAYLTSKIDPTTGFVNLFYIKYENIEKAKQFFDGFDFIRFNKKDDMFEFSFDYKSFTQKACGIRSKWIVYTNGERIIKYPNPEKNNLFDEKVINVTDEIKGLFKQYRIPYENGEDIKEIIISKAEADFYKRLFRLLHQTLQMRNSTSDGTRDYIISPVKNDRGEFFCSEFSEGTMPKDADANGAYNIARKGLWVLEQIRQKDEGEKVNLSMTNAEWLKYAQLHLL

SEQ ID NO: 350 - protein, 1115 aa, synthetic construct
MGIHGVPAAATRSFILKIEPNEEVKKGLWKTHEVLNHGIAYYMNILKLIRQEAIYEHHEQDPKNPKKVSKAEIQAELWDFVLKMQKCNSFTHEVDKDEVFNILRELYEELVPSSVEKKGEANQLSNKFLYPLVDPNSQSGKGTASSGRKPRWYNLKIAGDPSWEEEKKKWEEDKKKDPLAKILGKLAEYGLIPLFIPYTDSNEPIVKEIKWMEKSRNQSVRRLDKDMFIQALERFLSWESWNLKVKEEYEKVEKEYKTLEERIKEDIQALKALEQYEKERQEQLLRDTLNTNEYRLSKRGLRGWREIIQKWLKMDENEPSEKYLEVFKDYQRKHPREAGDYSVYEFLSKKENHFIWRNHPEYPYLYATFCEIDKKKKDAKQQATFTLADPINHPLWVRFEERSGSNLNKYRILTEQLHTEKLKKKLTVQLDRLIYPTESGGWEEKGKVDIVLLPSRQFYNQIFLDIEEKGKHAFTYKDESIKFPLKGTLGGARVQFDRDHLRRYPHKVESGNVGRIYFNMTVNIEPTESPVSKSLKIHRDDFPKVVNFKPKELTEWIKDSKGKKLKSGIESLEIGLRVMSIDLGQRQAAAASIFEVVDQKPDIEGKLFFPIKGTELYAVHRASFNIKLPGETLVKSREVLRKAREDNLKLMNQKLNFLRNVLHFQQFEDITEREKRVTKWISRQENSDVPLVYQDELIQIRELMYKPYKDWVAFLKQLHKRLEVEIGKEVKHWRKSLSDGRKGLYGISLKNIDEIDRTRKFLLRWSLRPTEPGEVRRLEPGQRFAIDQLNHLNALKEDRLKKMANTIIMHALGYCYDVRKKKWQAKNPACQIILFEDLSNYNPYGERSRFENSRLMKWSRREIPRQVALQGEIYGLQVGEVGAQFSSRFHAKTGSPGIRCRVVTKEKLQDNRFFKNLQREGRLTLDKIAVLKEGDLYPDKGGEKFISLSKDRKCVTTHADINAAQNLQKRFWTRTHGFYKVYCKAYQVDGQTVYIPESKDQKQKIIEEFGEGYFILKDGVYEWVNAGKLKIKKGSSKQSSSELVDSDILKDSFDLASELKGEKLMLYRDPSGNVFPSDKWMAAGVFFGKLERILISKLTNQYSISTIEDDSSKQS

SEQ ID NO: 351 - protein, 1147 aa, synthetic construct
MAVKSIKVKLRLSECPDILAGMWQLHRATNAGVRYYTEWVSLMRQEILYSRGPDGGQQCYMTAEDCQRELLRRLRNRQLHNGRQDQPGTDADLLAISRRLYEILVLQSIGKRGDAQQIASSFLSPLVDPNSKGGRGEAKSGRKPAWQKMRDQGDPRWVAAREKYEQRKAVDPSKEILNSLDALGLRPLFAVFTETYRSGVDWKPLGKSQGVRTWDRDMFQQALERLMSWESWNRRVGEEYARLFQQKMKFEQEHFAEQSHLVKLARALEADMRAASQGFEAKRGTAHQITRRALRGADRVFEIWKSIPEEALFSQYDEVIRQVQAEKRRDFGSHDLFAKLAEPKYQPLWRADETFLTRYALYNGVLRDLEKARQFATFTLPDACVNPIWTRFESSQGSNLHKYEFLFDHLGPGRHAVRFQRLLVVESEGAKERDSVVVPVAPSGQLDKLVLREEEKSSVALHLHDTARPDGFMAEWAGAKLQYERSTLARKARRDKQGMRSWRRQPSMLMSAAQMLEDAKQAGDVYLNISVRVKSPSEVRGQRRPPYAALFRIDDKQRRVTVNYNKLSAYLEEHPDKQIPGAPGLLSGLRVMSVDLGLRTSASISVFRVAKKEEVEALGDGRPPHYYPIHGTDDLVAVHERSHLIQMPGETETKQLRKLREERQAVLRPLFAQLALLRLLVRCGAADERIRTRSWQRLTKQGREFTKRLTPSWREALELELTRLEAYCGRVPDDEWSRIVDRTVIALWRRMGKQVRDWRKQVKSGAKVKVKGYQLDVVGGNSLAQIDYLEQQYKFLRRWSFFARASGLVVRADRESHFAVALRQHIENAKRDRLKKLADRILMEALGYVYEASGPREGQWTAQHPPCQLIILEELSAYRFSDDRPPSENSKLMAWGHRGILEELVNQAQVHDVLVGTVYAAFSSRFDARTGAPGVRCRRVPARFVGATVDDSLPLWLTEFLDKHRLDKNLLRPDDVIPTGEGEFLVSPCGEEAARVRQVHADINAAQNLQRRLWQNFDITELRLRCDVKMGGEGTVLVPRVNNARAKQLFGKKVLVSQDGVTFFERSQTGGKPHSEKQTDLTDKELELIAEADEARAKSVVLFRDPSGHIGKGHWIRQREFWSLVKQRIESHTAERIRVRGVGSSLD

SEQ ID NO: 352 - protein, 1108 aa, synthetic construct
MAIRSIKLKLKTHTGPEAQNLRKGIWRTHRLLNEGVAYYMKMLLLFRQESTGERPKEELQEELICHIREQQQRNQADKNTQALPLDKALEALRQLYELLVPSSVGQSGDAQIISRKFLSPLVDPNSEGGKGTSKAGAKPTWQKKKEANDPTWEQDYEKWKKRREEDPTASVITTLEEYGIRPIFPLYTNTVTDIAWLPLQSNQFVRTWDRDMLQQAIERLLSWESWNKRVQEEYAKLKEKMAQLNEQLEGGQEWISLLEQYEENRERELRENMTAANDKYRITKRQMKGWNELYELWSTFPASASHEQYKEALKRVQQRLRGRFGDAHFFQYLMEEKNRLIWKGNPQRIHYFVARNELTKRLEEAKQSATMTLPNARKHPLWVRFDARGGNLQDYYLTAEADKPRSRRFVTFSQLIWPSESGWMEKKDVEVELALSRQFYQQVKLLKNDKGKQKIEFKDKGSGSTFNGHLGGAKLQLERGDLEKEEKNFEDGEIGSVYLNVVIDFEPLQEVKNGRVQAPYGQVLQLIRRPNEFPKVTTYKSEQLVEWIKASPQHSAGVESLASGFRVMSIDLGLRAAAATSIFSVEESSDKNAADFSYWIEGTPLVAVHQRSYMLRLPGEQVEKQVMEKRDERFQLHQRVKFQIRVLAQIMRMANKQYGDRWDELDSLKQAVEQKKSPLDQTDRTFWEGIVCDLTKVLPRNEADWEQAVVQIHRKAEEYVGKAVQAWRKRFAADERKGIAGLSMWNIEELEGLRKLLISWSRRTRNPQEVNRFERGHTSHQRLLTHIQNVKEDRLKQLSHAIVMTALGYVYDERKQEWCAEYPACQVILFENLSQYRSNLDRSTKENSTLMKWAHRSIPKYVHMQAEPYGIQIGDVRAEYSSRFYAKTGTPGIRCKKVRGQDLQGRRFENLQKRLVNEQFLTEEQVKQLRPGDIVPDDSGELFMTLTDGSGSKEVVFLQADINAAHNLQKRFWQRYNELFKVSCRVIVRDEEEYLVPKTKSVQAKLGKGLFVKKSDTAWKDVYVWDSQAKLKGKTTFTEESESPEQLEDFQEIIEEAEEAKGTYRTLFRDPSGVFFPESVWYPQKDFWGEVKRKLYGKLRERFLTKAR

SEQ ID NO: 353 - protein, 1146 aa, synthetic construct
MNVAVKSIKVKLMLGHLPEIREGLWHLHEAVNLGVRYYTEWLALLRQGNLYRRGKDGAQECYMTAEQCRQELLVRLRDRQKRNGHTGDPGTDEELLGVARRLYELLVPQSVGKKGQAQMLASGFLSPLADPKSEGGKGTSKSGRKPAWMGMKEAGDSRWVEAKARYEANKAKDPTKQVIASLEMYGLRPLFDVFTETYKTIRWMPLGKHQGVRAWDRDMFQQSLERLMSWESWNERVGAEFARLVDRRDRFREKHFTGQEHLVALAQRLEQEMKEASPGFESKSSQAHRITKRALRGADGIIDDWLKLSEGEPVDRFDEILRKRQAQNPRRFGSHDLFLKLAEPVFQPLWREDPSFLSRWASYNEVLNKLEDAKQFATFTLPSPCSNPVWARFENAEGTNIFKYDFLFDHFGKGRHGVRFQRMIVMRDGVPTEVEGIVVPIAPSRQLDALAPNDAASPIDVFVGDPAAPGAFRGQFGGAKIQYRRSALVRKGRREEKAYLCGFRLPSQRRTGTPADDAGEVFLNLSLRVESQSEQAGRRNPPYAAVFHISDQTRRVIVRYGEIERYLAEHPDTGIPGSRGLTSGLRVMSVDLGLRTSAAISVFRVAHRDELTPDAHGRQPFFFPIHGMDHLVALHERSHLIRLPGETESKKVRSIREQRLDRLNRLRSQMASLRLLVRTGVLDEQKRDRNWERLQSSMERGGERMPSDWWDLFQAQVRYLAQHRDASGEAWGRMVQAAVRTLWRQLAKQVRDWRKEVRRNADKVKIRGIARDVPGGHSLAQLDYLERQYRFLRSWSAFSVQAGQVVRAERDSRFAVALREHIDNGKKDRLKKLADRILMEALGYVYVTDGRRAGQWQAVYPPCQLVLLEELSEYRFSNDRPPSENSQLMVWSHRGVLEELIHQAQVHDVLVGTIPAAFSSRFDARTGAPGIRCRRVPSIPLKDAPSIPIWLSHYLKQTERDAAALRPGELIPTGDGEFLVTPAGRGASGVRVVHADINAAHNLQRRLWENFDLSDIRVRCDRREGKDGTVVLIPRLTNQRVKERYSGVIFTSEDGVSFTVGDAKTRRRSSASQGEGDDLSDEEQELLAEADDARERSVVLFRDPSGFVNGGRWTAQRAFWGMVHNRIETLLAERFSVSGAAEKVRG

SEQ ID NO: 354 - protein, 1129 aa, synthetic construct
MAVKSMKVKLRLDNMPEIRAGLWKLHTEVNAGVRYYTEWLSLLRQENLYRRSPNGDGEQECYKTAEECKAELLERLRARQVENGHCGPAGSDDELLQLARQLYELLVPQAIGAKGDAQQIARKFLSPLADKDAVGGLGIAKAGNKPRWVRMREAGEPGWEEEKAKAEARKSTDRTADVLRALADFGLKPLMRVYTDSDMSSVQWKPLRKGQAVRTWDRDMFQQAIERMMSWESWNQRVGEAYAKLVEQKSRFEQKNFVGQEHLVQLVNQLQQDMKEASHGLESKEQTAHYLTGRALRGSDKVFEKWEKLDPDAPFDLYDTEIKNVQRRNTRRFGSHDLFAKLAEPKYQALWREDASFLTRYAVYNSIVRKLNHAKMFATFTLPDATAHPIWTRFDKLGGNLHQYTFLFNEFGEGRHAIRFQKLLTVEDGVAKEVDDVTVPISMSAQLDDLLPRDPHELVALYFQDYGAEQHLAGEFGGAKIQYRRDQLNHLHARRGARDVYLNLSVRVQSQSEARGERRPPYAAVFRLVGDNHRAFVHFDKLSDYLAEHPDDGKLGSEGLLSGLRVMSVDLGLRTSASISVFRVARKDELKPNSEGRVPFCFPIEGNENLVAVHERSQLLKLPGETESKDLRAIREERQRTLRQLRTQLAYLRLLVRCGSEDVGRRERSWAKLIEQPMDANQMTPDWREAFEDELQKLKSLYGICGDREWTEAVYESVRRVWRHMGKQVRDWRKDVRSGERPKIRGYQKDVVGGNSIEQIEYLERQYKFLKSWSFFGKVSGQVIRAEKGSRFAITLREHIDHAKEDRLKKLADRIIMEALGYVYALDDERGKGKWVAKYPPCQLILLEELSEYQFNNDRPPSENNQLMQWSHRGVFQELLNQAQVHDLLVGTMYAAFSSRFDARTGAPGIRCRRVPARCAREQNPEPFPWWLNKFVAEHKLDGCPLRADDLIPTGEGEFFVSPFSAEEGDFHQIHADLNAAQNLQRRLWSDFDISQIRLRCDWGEVDGEPVLIPRTTGKRTADSYGNKVFYTKTGVTYYERERGKKRRKVFAQEELSEEEAELLVEADEAREKSVVLMRDPSGIINRGDWTRQKEFWSMVNQRIEGYLVKQIRSRVRLQESACENTGDI

SEQ ID NO: 355 - protein, 966 aa, synthetic construct
MIEKKKSFAKGMGVKSTLVSGSKVYMTTFAEGSDARLEKIVEGDSIRSVNEGEAFSAEMADKNAGYKIGNAKFSHPKGYAVVANNPLYTGPVQQDMLGLKETLEKRYFGESADGNDNICIQVIHNILDIEKILAEYITNAAYAVNNISGLDKDIIGFGKFSTVYTYDEFKDPEHHRAAFNNNDKLINAIKAQYDEFDNFLDNPRLGYFGQAFFSKEGRNYIINYGNECYDILALLSGLRHWVVHNNEEESRISRTWLYNLDKNLDNEYISTLNYLYDRITNELTNSFSKNSAANVNYIAETLGINPAEFAEQYFRFSIMKEQKNLGFNITKLREVMLDRKDMSEIRKNHKVFDSIRTKVYTMMDFVIYRYYIEEDAKVAAANKSLPDNEKSLSEKDIFVINLRGSFNDDQKDALYYDEANRIWRKLENIMHNIKEFRGNKTREYKKKDAPRLPRILPAGRDVSAFSKLMYALTMFLDGKEINDLLTTLINKFDNIQSFLKVMPLIGVNAKFVEEYAFFKDSAKIADELRLIKSFARMGEPIADARRAMYIDAIRILGTNLSYDELKALADTFSLDENGNKLKKGKHGMRNFIINNVISNKRFHYLIRYGDPAHLHEIAKNEAVVKFVLGRIADIQKKQGQNGKNQIDRYYETCIGKDKGKSVSEKVDALTKIITGMNYDQFDKKRSVIEDTGRENAEREKFKKIISLYLTVIYHILKNIVNINARYVIGFHCVERDAQLYKEKGYDINLKKLEEKGFSSVTKLCAGIDETAPDKRKDVEKEMAERAKESIDSLESANPKLYANYIKYSDEKKAEEFTRQINREKAKTALNAYLRNTKWNVIIREDLLRIDNKTCTLFRNKAVHLEVARYVHAYINDIAEVNSYFQLYHYIMQRIIMNERYEKSSGKVSEYFDAVNDEKKYNDRLLKLLCVPFGYCIPRFKNLSIEALFDRNEAAKFDKEKKKVSGN

SEQ ID NO: 356 - protein, 1152 aa, synthetic construct
MKVTKVDGISHKKYIEEGKLVKSTSEENRTSERLSELLSIRLDIYIKNPDNASEEENRIRRENLKKFFSNKVLHLKDSVLYLKNRKEKNAVQDKNYSEEDISEYDLKNKNSFSVLKKILLNEDVNSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIREYYHKIIGRKNDKENFAKIIYEEIQNVNNIKELIEKIPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQVGEIATSDFIARNRQNEAFLRNIIGVSSVAYFSLRNILETENENGITGRMRGKTVKNNKGEEKYVSGEVDKIYNENKQNEVKENLKMFYSYDFNMDNKNEIEDFFANIDEAISSIRHGIVHFNLELEGKDIFAFKNIAPSEISKKMFQNEINEKKLKLKIFKQLNSANVFNYYEKDVIIKYLKNTKFNFVNKNIPFVPSFTKLYNKIEDLRNTLKFFWSVPKDKEEKDAQIYLLKNIYYGEFLNKFVKNSKVFFKITNEVIKINKQRNQKTGHYKYQKFENIEKTVPVEYLAIIQSREMINNQDKEEKNTYIDFIQQIFLKGFIDYLNKNNLKYIESNNNNDNNDIFSKIKIKKDNKEKYDKILKNYEKHNRNKEIPHEINEFVREIKLGKILKYTENLNMFYLILKLLNHKELTNLKGSLEKYQSANKEETFSDELELINLLNLDNNRVTEDFELEANEIGKFLDFNENKIKDRKELKKFDTNKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENHYIEEIFNFDNSKNVKYKSGQIVEKYINFYKELYKDNVEKRSIYSDKKVKKLKQEKKDLYIRNYIAHFNYIPHAEISLLEVLENLRKLLSYDRKLKNAIMKSIVDILKEYGFVATFKIGADKKIEIQTLESEKIVHLKNLKKKKLMTDRNSEELCELVKVMFEYKALE

SEQ ID NO: 357 - protein, 1094 aa, synthetic construct
MNIPALVENQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNHYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKLPKDRIHSEKSNKSVAMDMLNEVKRCPDELFTTLSAEKQSRFRIISDDHNEVLMKRSSDRFVPLLLQYIDYGKLFDHIRFHVNMGKLRYLLKADKTCIDGQTRVRVIEQPLNGFGRLEEAETMRKQENGTFGNSGIRIRDFENMKRDDANPANYPYIVDTYTHYILENNKVEMFINDKEDSAPLLPVIEDDRYVVKTIPSCRMSTLEIPAMAFHMFLFGSKKTEKLIVDVHNRYKRLFQAMQKEEVTAENIASFGIAESDLPQKILDLISGNAHGKDVDAFIRLTVDDMLTDTERRIKRFKDDRKSIRSADNKMGKRGFKQISTGKLADFLAKDIVLFQPSVNDGENKITGLNYRIMQSAIAVYDSGDDYEAKQQFKLMFEKARLIGKGTTEPHPFLYKVFARSIPANAVEFYERYLIERKFYLTGLSNEIKKGNRVDVPFIRRDQNKWKTPAMKTLGRIYSEDLPVELPRQMFDNEIKSHLKSLPQMEGIDFNNANVTYLIAEYMKRVLDDDFQTFYQWNRNYRYMDMLKGEYDRKGSLQHCFTSVEEREGLWKERASRTERYRKQASNKIRSNRQMRNASSEEIETILDKRLSNSRNEYQKSEKVIRRYRVQDALLFLLAKKTLTELADFDGERFKLKEIMPDAEKGILSEIMPMSFTFEKGGKKYTITSEGMKLKNYGDFFVLASDKRIGNLLELVGSDIVSKEDIMEEFNKYDQCRPEISSIVFNLEKWAFDTYPELSARVDREEKVDFKSILKILLNNKNINKEQSDILRKIRNAFDHNNYPDKGVVEIKALPEIAMSIKKAFGEYAIMKGSLQ

SEQ ID NO: 358 - protein, 1179 aa, synthetic construct
MTEQSERPYNGTYYTLEDKHFWAAFLNLARHNAYITLTHIDRQLAYSKADITNDQDVLSFKALWKNFDNDLERKSRLRSLILKHFSFLEGAAYGKKLFESKSSGNKSSKNKELTKKEKEELQANALSLDNLKSILFDFLQKLKDFRNYYSHYRHSGSSELPLFDGNMLQRLYNVFDVSVQRVKIDHEHNDEVDPHYHFNHLVRKGKKDRYGHNDNPSFKHHFVDGEGMVTEAGLLFFVSLFLEKRDAIWMQKKIRGFKGGTETYQQMTNEVFCRSRISLPKLKLESLRMDDWMLLDMLNELVRCPKPLYDRLREDDRACFRVPVDILPDEDDTDGGGEDPFKNTLVRHQDRFPYFALRYFDLKKVFTSLRFHIDLGTYHFAIYKKMIGEQPEDRHLTRNLYGFGRIQDFAEEHRPEEWKRLVRDLDYFETGDKPYISQTSPHYHIEKGKIGLRFMPEGQHLWPSPEVGTTRTGRSKYAQDKRLTAEAFLSVHELMPMMFYYFLLREKYSEEVSAERVQGRIKRVIEDVYAVYDAFARDEINTRDELDACLADKGIRRGHLPRQMIAILSQEHKDMEEKIRKKLQEMMADTDHRLDMLDRQTDRKIRIGRKNAGLPKSGVIADWLVRDMMRFQPVAKDASGKPLNNSKANSTEYRMLQRALALFGGEKERLTPYFRQMNLTGGNNPHPFLHETRWESHTNILSFYRSYLRARKAFLERIGRSDRVENRPFLLLKEPKTDRQTLVAGWKGEFHLPRGIFTEAVRDCLIEMGHDEVASYKEVGFMAKAVPLYFERACEDRVQPFYDSPFNVGNSLKPKKGRFLSKEERAEEWERGKERFRDLEAWSYSAARRIEDAFAGIEYASPGNKKKIEQLLRDLSLWEAFESKLKVRADRINLAKLKKEILEAQEHPYHDFKSWQKFERELRLVKNQDIITWMMCRDLMEENKVEGLDTGTLYLKDIRPNVQEQGSLNVLNRVKPMRLPVVVYRADSRGHVHKEEAPLATVYIEERDTKLLKQGNFKSFVKDRRLNGLFSFVDTGGLAMEQYPISKLRVEYELAKYQTARVCVFELTLRLEESLLTRYPHLPDESFREMLESWSDPLLAKWPELHGKVRLLIAVRNAFSHNQYPMYDEAVFSSIRKYDPSSPDAIEERMGLNIAHRLSEEVKQAKETVERIIQAGSLQ

SEQ ID NO: 359 - protein, 1111 aa, synthetic construct
MEKPLLPNVYTLKHKFFWGAFLNIARHNAFITICHINEQLGLKTPSNDDKIVDVVCETWNNILNNDHDLLKKSQLTELILKHFPFLTAMCYHPPKKEGKKKGHQKEQQKEKESEAQSQAEALNPSKLIEALEILVNQLHSLRNYYSHYKHKKPDAEKDIFKHLYKAFDASLRMVKEDYKAHFTVNLTRDFAHLNRKGKNKQDNPDFNRYRFEKDGFFTESGLLFFTNLFLDKRDAYWMLKKVSGFKASHKQREKMTTEVFCRSRILLPKLRLESRYDHNQMLLDMLSELSRCPKLLYEKLSEENKKHFQVEADGFLDEIEEEQNPFKDTLIRHQDRFPYFALRYLDLNESFKSIRFQVDLGTYHYCIYDKKIGDEQEKRHLTRTLLSFGRLQDFTEINRPQEWKALTKDLDYKETSNQPFISKTTPHYHITDNKIGFRLGTSKELYPSLEIKDGANRIAKYPYNSGFVAHAFISVHELLPLMFYQHLTGKSEDLLKETVRHIQRIYKDFEEERINTIEDLEKANQGRLPLGAFPKQMLGLLQNKQPDLSEKAKIKIEKLIAETKLLSHRLNTKLKSSPKLGKRREKLIKTGVLADWLVKDFMRFQPVAYDAQNQPIKSSKANSTEFWFIRRALALYGGEKNRLEGYFKQTNLIGNTNPHPFLNKFNWKACRNLVDFYQQYLEQREKFLEAIKNQPWEPYQYCLLLKIPKENRKNLVKGWEQGGISLPRGLFTEAIRETLSEDLMLSKPIRKEIKKHGRVGFISRAITLYFKEKYQDKHQSFYNLSYKLEAKAPLLKREEHYEYWQQNKPQSPTESQRLELHTSDRWKDYLLYKRWQHLEKKLRLYRNQDVMLWLMTLELTKNHFKELNLNYHQLKLENLAVNVQEADAKLNPLNQTLPMVLPVKVYPATAFGEVQYHKTPIRTVYIREEHTKALKMGNFKALVKDRRLNGLFSFIKEENDTQKHPISQLRLRRELEIYQSLRVDAFKETLSLEEKLLNKHTSLSSLENEFRALLEEWKKEYAASSMVTDEHIAFIASVRNAFCHNQYPFYKEALHAPIPLFTVAQPTTEEKDGLGIAEALLKVLREYCEIVKSQIGSSLQKKLEELELGSS

SEQ ID NO: 360 - RNA, 3585 nt, Tick-borne encephalitis virus
  source 1..3585: mol_type = mRNA
gggggacagatcgcctggagacgccatccacgctgttttgacctccatagaagacaccgggaccgatccagcctccgcggccgggaacggtgcattggaacgcggattccccgtgccaagagtgactcaccgtccttgacacggccaccatggcctctaacttcacccagtttgtgctggtggacaatggaggaaccggcgatgtgacagtggcaccatccaactttgccaatggcatcgccgagtggatcagctccaactctaggagccaggcctacaaggtgacctgcagcgtgcggcagtctagcgcccagaatagaaagtacacaatcaaggtggaggtgccaaagggagcatggcgcagctatctgaacatggagctgaccatccccatcttcgccacaaattccgactgtgagctgatcgtgaaggccatgcagggcctgctgaaggatggcaaccccatcccttctgccatcgccgccaatagcggcatctactctggaggatctagcggaggcagctctggcagcgagacaccaggaacaagcgagtcagcaacaccagagagcagtggcggcagcagcggcggcagcagcggcggatccaagcggcccgccgccaccaagaaggccggccaggccaagaagaagaagatgaccaacctgtctgacatcatcgagaaggagacaggcaagcagctggtcatccaggagagcatcctgatgctgcccgaagaagtcgaagaagtgatcggaaacaagcctgagagcgatatcctggtccataccgcctacgacgagagtaccgacgaaaatgtgatgctgctgacatccgacgccccagagtataagccctgggctctggtcatccaggattccaacggagagaacaaaatcaaaatgctgtctggaggcagcatgggaccattttgcctgggctgttctcaccggaagtgctatagccccatcagaaacctgatcagccaggagacattcaagtttcacttcaagaatctgggctacgccaagggccggaaggataccttcctgtgctatgaggtgacaagaaaggactgtgatagccccgtgtccctgcaccacggcgtgtttaagaacaaggacaatatccacgccgagatctgttttctgtactggttccacgataaggtgctgaaggtgctgtcccccagggaggagttcaagatcacctggtatatgtcttggagcccttgcttcgagtgtgccgagcagatcgtgcgctttctggccacacaccacaacctgagcctggacatctttagctcccggctgtacaacgtgcaggatcctgagacacagcagaatctgtgcagactggtgcaggagggagcacaggtggcagcaatggacctgtatgagttcaagaagtgttggaagaagtttgtggataacggcggccggagattccggccgtggaagagactgctgaccaacttccggtaccaggacagcaagctgcaggagatcctgcgcccttgctatatctccgtgccatctagcgagaatctgtacttccagagcatgtcctctagcaccctgtctaatatctgcctgaccaagggcctgcccgagacaaggttctgggtggagggccggagaatggaccctctgagcgaggaggagttttactcccagttctataaccagagggtgaagcacctgtgctactatcaccgcatgaagccttacctgtgctatcagctggagcagttcaatggacaggcaccactgaagggatgcctgctgtccgagaagggcaagcagcacgccgagatcctgtttctggataagatcagatctatggagctgagccaggtgaccatcacatgttacctgacatggtccccatgcccaaactgtgcatggcagctggcagccttcaagagggaccgcccagatctgatcctgcacatctacacctctaggctgtattttcactggaagcgccccttccagaagggcctgtgctccctgtggcagtctggcatcctggtggacgtgatggatctgccccagtttaccgactgttggacaaacttcgtgaatcctaagcggccattttggccctggaagggcctggagatcatcagcaggcgcacacagcggagactgaggcgcatcaaggagtcctggggcctgcaggacctggtgaacgattttggcaatctgcagctgggaccccctatgagcaagcggcccgccgccaccaagaaggccggccaggccaagaagaagaaggctactaacttcagcctgctgaagcaggctggggacgtggaggagaaccctggacctatgggcaacgccagaacaagacggcgggagcgcagagccgagaagcaggcccaatggaaagccgctaattccggcgggagcagtggcggatccagtgggtccgaaacccctggcacaagcgaaagcgctacccctgagtccagcggagggtcctcaggggaattcatgaaaagcatgtctagcatggtgtctgacacaagctgtaccttcccctcctctgatggcatcttttggaagcactggattcagacaaaggacggccagtgtggctcccctctggtgtctaccagggatggcttcatcgtgggcatccacagcgcctccaacttcacaaacaccaacaactactttacatccgtgcctaagaacttcatggagctgctgaccaatcaggaggcacagcagtgggtgtctggatggcgcctgaacgccgatagcgtgctgtggggcggccacaaggtgtttatggtgaagccagaggagcccttccagcctgtgaaggaggccacccagaagcggcccgccgccaccaagaaggccggccaggccaagaagaagaaggagggcagaggaagtctgctaacatgcggtgacgtcgaggagaatcctggcccaatgggcgagtccctgtttaagggcccacgggactacaaccccatcagctccacaatctgccacctgaccaatgagagcgatggccacaccacatccctgtatggcatcggcttcggccccttcatcatcacaaacaagcacctgtttcggagaaacaatggcaccctgctggtgcagtctctgcacggcgtgttcaaggtgaagaataccacaaccctgcagcagcacctgatcgacggaagggatatgatcatcatcagaatgcctaaggacttccccccttttccacagaagctgaagtttcgggagccacagagggaggagaggatctgcctggtgacaaccaacttccagacactcgagaagcggcccgccgccaccaagaaggccggccaggccaagaagaagaagtaatgagctccgggtggcatccctgtgacccctccccagtgcctctcctggccctggaagttgccactccagtgcccaccagccttgtcctaataaaattaagttgcatcaagctgcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 361 - RNA, 4593 nt, synthetic construct
  source 1..4593: mol_type = other RNA
gggggacagatcgcctggagacgccatccacgctgttttgacctccatagaagacaccgggaccgatccagcctccgcggccgggaacggtgcattggaacgcggattccccgtgccaagagtgactcaccgtccttgacacggccaccatggcccctgccgccaagcgggtcaagctcgacggtatccacggagtcccagcagctatggacaagaagtacagcatcggcctggccatcggcaccaacagcgtgggctgggccgtgatcaccgacgagtacaaggtgcccagcaagaagttcaaggtgctgggcaacaccgaccggcacagcatcaagaagaacctgatcggcgccctgctgttcgacagcggcgagaccgccgaggccacccggctgaagcggaccgcccggcggcggtacacccggcggaagaaccggatctgctacctgcaggagatcttcagcaacgagatggccaaggtggacgacagcttcttccaccggctggaggagagcttcctggtggaggaggacaagaagcacgagcggcaccccatcttcggcaacatcgtggacgaggtggcctaccacgagaagtaccccaccatctaccacctgcggaagaagctggtggacagcaccgacaaggccgacctgcggctgatctacctggccctggcccacatgatcaagttccggggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccggctgagcaagagccggcggctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgggtgaacaccgagatcaccaaggcccccctgagcgccagcatgatcaagcggtacgacgagcaccaccaggacctgaccctgctgaaggccctggtgcggcagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgggaggacctgctgcggaagcagcggaccttcgacaacggcagcatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaacagccggttcgcctggatgacccgaaagagcgaggagaccatcaccccctggaacttcgaggaggtggtggacaagggcgccagcgcccagagcttcatcgagcggatgaccaacttcgacaagaacctgcccaacgagaaggtgctgcccaagcacagcctgctgtacgagtacttcaccgtgtacaacgagctgaccaaggtgaagtacgtgaccgagggcatgcggaagcccgccttcctgagcggcgagcagaagaaggccatcgtggacctgctgttcaagaccaaccggaaggtgaccgtgaagcagctgaaggaggactacttcaagaagatcgagtgcttcgacagcgtggagatcagcggcgtggaggaccggttcaacgccagcctgggcacctaccacgacctgctgaagatcatcaaggacaaggacttcctggacaacgaggagaacgaggacatcctggaggacatcgtgctgaccctgaccctgttcgaggaccgggagatgatcgaggagcggctaaagacctacgcccacctgttcgacgacaaggtgatgaagcagctgaagcggcggcggtacaccggctggggccggctgagccggaagctgatcaacggcatccgggacaagcagagcggcaagaccatcctggacttcctcaagagcgacggcttcgccaaccggaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaacagccgggagcggatgaagcggatcgaggagggcatcaaggagctgggcagccagatcctgaaggagcaccccgtggagaacacccagctgcagaacgagaagctgtacctgtactacctgcagaacggccgggacatgtacgtggaccaggagctggacatcaaccggctgagcgactacgacgtggaccacatcgtgccccagagcttcctgaaggacgacagcatcgacaacaaggtgctgacccggagcgacaagaaccggggcaagagcgacaacgtgcccagcgaggaggtggtgaagaagatgaagaactactggcggcagctgctgaacgccaagctgatcacccagcggaagttcgacaacctgaccaaggccgagcggggcggcctgagcgagctggacaaggccggcttcatcaagcggcagctggtggagacccggcagatcaccaagcacgtggcccagatcctggacagccggatgaacaccaagtacgacgagaacgacaagctgatccgggaggtgaaggtgatcaccctcaagagcaagctggtgagcgacttccggaaggacttccagttctacaaggtgcgggagatcaacaactaccaccacgcccacgacgcctacctgaacgccgtggtgggcaccgccctgatcaagaagtaccccaagctggagagcgagttcgtgtacggcgactacaaggtgtacgacgtgcggaagatgatcgccaagagcgagcaggagatcggcaaggccaccgccaagtacttcttctacagcaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgagcatgccccaggtgaacatcgtgaaaaagaccgaggtgcagaccggcggcttcagcaaggagagcatcctgcccaagcggaacagcgacaagctgatcgcccggaagaaggactgggaccccaagaagtacggcggcttcgacagccccaccgtggcctacagcgtgctggtggtggccaaggtggagaagggcaagagcaagaagctcaagagcgtgaaggagctgctgggcatcaccatcatggagcggagcagcttcgagaagaaccccatcgacttcctggaggccaagggctacaaggaggtgaagaaggacctgatcatcaagctgcccaagtacagcctgttcgagctggagaacggccggaagcggatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgggtgatcctggccgacgccaacctggacaaggtgctgagcgcctacaacaagcaccgggacaagcccatccgggagcaggccgagaacatcatccacctgttcaccctgaccaacctgggcgcccccgccgccttcaagtacttcgacaccaccatcgaccggaagcggtacaccagcaccaaggaggtgctggacgccaccctgatccaccagagcatcaccggcctgtacgagacccggatcgacctgagccagctgggcggcgacagcggcggcagcaagcggcccgccgccaccaagaaggccggccaggccaagaagaagaagtaatgagctccgggtggcatccctgtgacccctccccagtgcctctcctggccctggaagttgccactccagtgcccaccagccttgtcctaataaaattaagttgcatcaagctgcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 362 - RNA, 116 nt, synthetic construct
  source 1..116: mol_type = other RNA
  modified_base 1..3: mod_base = OTHER; note = 2'-O-methyl
  modified_base 114..116: mod_base = OTHER; note = 2'-O-methyl
  modified_base 1..4: mod_base = OTHER; note = phosphorothioate linkage
  modified_base 113..116: mod_base = OTHER; note = phosphorothioate linkage
accaggggaggtttgagagctaggccaacatgaggatcacccatgtctgcagggcctagcaagttcaaataaggctagtccgttatcaacttgaaaaagtggcaccgagtcggtgc

SEQ ID NO: 363 - RNA, 111 nt, synthetic construct
  source 1..111: mol_type = other RNA
  modified_base 1..3: mod_base = OTHER; note = 2'-O-methyl
  modified_base 109..111: mod_base = OTHER; note = 2'-O-methyl
  modified_base 1..4: mod_base = OTHER; note = phosphorothioate linkage
  modified_base 108..111: mod_base = OTHER; note = phosphorothioate linkage
caggttccatgggatgctctgtttgagagctagggccctgaagaagggccctagcaagttcaaataaggctagtccgttatcaacttgaaaaagtggcaccgagtcggtgc

SEQ ID NO: 364 - RNA, 69 nt, Mus musculus
  source 1..69: mol_type = other RNA
  modified_base 1..3: mod_base = OTHER; note = 2'-O-methyl
  modified_base 67..69: mod_base = OTHER; note = 2'-O-methyl
  modified_base 1..4: mod_base = OTHER; note = phosphorothioate linkage
  modified_base 66..69: mod_base = OTHER; note = phosphorothioate linkage
gaagaggaaagtttgagagctatgctgttttgggccaacatgaggatcacccatgtctgcagggccttt

SEQ ID NO: 365 - RNA, 64 nt, Mus musculus
  source 1..64: mol_type = other RNA
  modified_base 1..3: mod_base = OTHER; note = 2'-O-methyl
  modified_base 62..64: mod_base = OTHER; note = 2'-O-methyl
  modified_base 1..4: mod_base = OTHER; note = phosphorothioate linkage
  modified_base 61..64: mod_base = OTHER; note = phosphorothioate linkage
actacaagttaaaaacgagggtttgagagctatgctgttttggggccctgaagaagggcccttt

SEQ ID NO: 366 - RNA, 69 nt, Homo sapiens
  source 1..69: mol_type = other RNA
  modified_base 1..3: mod_base = OTHER; note = 2'-O-methyl
  modified_base 67..69: mod_base = OTHER; note = 2'-O-methyl
  modified_base 1..4: mod_base = OTHER; note = phosphorothioate linkage
  modified_base 66..69: mod_base = OTHER; note = phosphorothioate linkage
gaagaagaaagtttgagagctatgctgttttgggccaacatgaggatcacccatgtctgcagggccttt

SEQ ID NO: 367 - RNA, 64 nt, Homo sapiens
  source 1..64: mol_type = other RNA
  modified_base 1..3: mod_base = OTHER; note = 2'-O-methyl
  modified_base 62..64: mod_base = OTHER; note = 2'-O-methyl
  modified_base 1..4: mod_base = OTHER; note = phosphorothioate linkage
  modified_base 61..64: mod_base = OTHER; note = phosphorothioate linkage
actacaagtcaaaaatgaaggtttgagagctatgctgttttggggccctgaagaagggcccttt

SEQ ID NO: 368 - RNA, 69 nt, Macaca mulatta; identical to SEQ ID NO: 366
  source 1..69: mol_type = other RNA
  modified_base 1..3: mod_base = OTHER; note = 2'-O-methyl
  modified_base 67..69: mod_base = OTHER; note = 2'-O-methyl
  modified_base 1..4: mod_base = OTHER; note = phosphorothioate linkage
  modified_base 66..69: mod_base = OTHER; note = phosphorothioate linkage

SEQ ID NO: 369 - RNA, 64 nt, Macaca mulatta; identical to SEQ ID NO: 367
  source 1..64: mol_type = other RNA
  modified_base 1..3: mod_base = OTHER; note = 2'-O-methyl
  modified_base 62..64: mod_base = OTHER; note = 2'-O-methyl
  modified_base 1..4: mod_base = OTHER; note = phosphorothioate linkage
  modified_base 61..64: mod_base = OTHER; note = phosphorothioate linkage

SEQ ID NO: 370 - RNA, 69 nt, Mus musculus; identical to SEQ ID NO: 302
  source 1..69: mol_type = other RNA

SEQ ID NO: 371 - RNA, 64 nt, Mus musculus; identical to SEQ ID NO: 303
  source 1..64: mol_type = other RNA

SEQ ID NO: 372 - RNA, 69 nt, Homo sapiens; identical to SEQ ID NO: 304
  source 1..69: mol_type = other RNA

SEQ ID NO: 373 - RNA, 64 nt, Homo sapiens; identical to SEQ ID NO: 305
  source 1..64: mol_type = other RNA

SEQ ID NO: 374 - RNA, 69 nt, Macaca mulatta; identical to SEQ ID NO: 304
  source 1..69: mol_type = other RNA

SEQ ID NO: 375 - RNA, 64 nt, Macaca mulatta; identical to SEQ ID NO: 303
  source 1..64: mol_type = other RNA

SEQ ID NO: 376 - RNA, 69 nt, Mus musculus; identical to SEQ ID NO: 364
  source 1..69: mol_type = other RNA

SEQ ID NO: 377 - RNA, 64 nt, Mus musculus; identical to SEQ ID NO: 365
  source 1..64: mol_type = other RNA

SEQ ID NO: 378 - RNA, 69 nt, Homo sapiens; identical to SEQ ID NO: 366
  source 1..69: mol_type = other RNA

SEQ ID NO: 379 - RNA, 64 nt, Homo sapiens; identical to SEQ ID NO: 367
  source 1..64: mol_type = other RNA

SEQ ID NO: 380 - RNA, 69 nt, Macaca mulatta; identical to SEQ ID NO: 366
  source 1..69: mol_type = other RNA

SEQ ID NO: 381 - RNA, 64 nt, Macaca mulatta; identical to SEQ ID NO: 367
  source 1..64: mol_type = other RNA

SEQ ID NO: 382 - RNA, 66 nt, synthetic construct; identical to SEQ ID NO: 13
  source 1..66: mol_type = other RNA

SEQ ID NO: 383 - RNA, 66 nt, synthetic construct; identical to SEQ ID NO: 14
  source 1..66: mol_type = other RNA

SEQ ID NO: 384 - RNA, 66 nt, synthetic construct; identical to SEQ ID NO: 15
  source 1..66: mol_type = other RNA

SEQ ID NO: 385 - RNA, 61 nt, synthetic construct; identical to SEQ ID NO: 16
  source 1..61: mol_type = other RNA

SEQ ID NO: 386 - RNA, 61 nt, synthetic construct; identical to SEQ ID NO: 17
  source 1..61: mol_type = other RNA

SEQ ID NO: 387 - RNA, 61 nt, synthetic construct; identical to SEQ ID NO: 18
  source 1..61: mol_type = other RNA

SEQ ID NO: 388 - RNA, 82 nt, synthetic construct; identical to SEQ ID NO: 19
  source 1..82: mol_type = other RNA

SEQ ID NO: 389 - RNA, 42 nt, synthetic construct; identical to SEQ ID NO: 20
  source 1..42: mol_type = other RNA

SEQ ID NO: 390 - RNA, 32 nt, synthetic construct; identical to SEQ ID NO: 21
  source 1..32: mol_type = other RNA

SEQ ID NO: 391 - RNA, 69 nt, synthetic construct; identical to SEQ ID NO: 154
  source 1..69: mol_type = other RNA
  modified_base 1..3: mod_base = OTHER; note = 2'-O-methyl
  modified_base 67..69: mod_base = OTHER; note = 2'-O-methyl
  modified_base 1..4: mod_base = OTHER; note = phosphorothioate linkage
  modified_base 66..69: mod_base = OTHER; note = phosphorothioate linkage

SEQ ID NO: 392 - RNA, 86 nt, synthetic construct; identical to SEQ ID NO: 155
  source 1..86: mol_type = other RNA
  modified_base 1..3: mod_base = OTHER; note = 2'-O-methyl
  modified_base 84..86: mod_base = OTHER; note = 2'-O-methyl
  modified_base 1..4: mod_base = OTHER; note = phosphorothioate linkage
  modified_base 83..86: mod_base = OTHER; note = phosphorothioate linkage

SEQ ID NO: 393 - RNA, 89 nt, synthetic construct; identical to SEQ ID NO: 156
  source 1..89: mol_type = other RNA
  modified_base 1..3: mod_base = OTHER; note = 2'-O-methyl
  modified_base 87..89: mod_base = OTHER; note = 2'-O-methyl
  modified_base 1..4: mod_base = OTHER; note = phosphorothioate linkage
  modified_base 86..89: mod_base = OTHER; note = phosphorothioate linkage

SEQ ID NO: 394 - RNA, 64 nt, synthetic construct; identical to SEQ ID NO: 157
  source 1..64: mol_type = other RNA
  modified_base 1..3: mod_base = OTHER; note = 2'-O-methyl
  modified_base 62..64: mod_base = OTHER; note = 2'-O-methyl
  modified_base 1..4: mod_base = OTHER; note = phosphorothioate linkage
  modified_base 61..64: mod_base = OTHER; note = phosphorothioate linkage

SEQ ID NO: 395 - RNA, 83 nt, synthetic construct; identical to SEQ ID NO: 158
  source 1..83: mol_type = other RNA
  modified_base 1..3: mod_base = OTHER; note = 2'-O-methyl
  modified_base 81..83: mod_base = OTHER; note = 2'-O-methyl
  modified_base 1..4: mod_base = OTHER; note = phosphorothioate linkage
  modified_base 80..83: mod_base = OTHER; note = phosphorothioate linkage